SEQUENCE LISTING (14 sequences)

SEQ ID NO: 1 - protein, 144 aa, Cardiovirus A
MSPNALDISRTYPTLHVLIQFNHRGLEVRLFRHGHFWAETRADVILRSKTKQVSFLSNGNYPSMDSRAPWNPWKNTYQAVLRAEPCRVTMDIYYKRVRPFRLPLVQKEWPVREENVFGLYRIFNAHYAGYFADLLIHDIETNPG

SEQ ID NO: 2 - DNA, 435 bp, Cardiovirus A
  source 1..435: mol_type = genomic DNA
atgagtccaaatgccctagacatttcaagaacataccccacgttacatgttctcattcaattcaaccatagaggtttggaggttagattgtttagacatggacacttttgggctgaaacacgtgcggacgtgattctgagatcaaagaccaaacaggtctctttcctgagcaacgggaactacccgtcaatggactctagagctccctggaatccttggaagaatacctaccaggcggttctaagagcagaaccatgtagagtgaccatggatatatattataagagagtcaggccttttagactgcccctggttcagaaggaatggcccgtgcgagaggagaacgttttcggtttgtaccggatcttcaatgcccactacgctggttactttgcggacctactgattcatgacattgagacaaatccagggtag

SEQ ID NO: 3 - skipped

SEQ ID NO: 4 - RNA, 13 nt, synthetic construct
  source 1..13: mol_type = other RNA
cagactcaaggag

SEQ ID NO: 5-6 - skipped (2 entries)

SEQ ID NO: 7 - RNA, 21 nt, synthetic construct
  source 1..21: mol_type = other RNA
gtcatcaatggctcaaaccct

SEQ ID NO: 8 - RNA, 55 nt, synthetic construct
  source 1..55: mol_type = other RNA
ggtttttcagactcaaggagcggcagtgtcatcaatggctcaaaccctactgccg

SEQ ID NO: 9-10 - skipped (2 entries)

SEQ ID NO: 11 - DNA, 239 bp, synthetic construct
  source 1..239: mol_type = other DNA
ctcctcctccggagggcggggtggagggtcgggggccctctctcccctctcccctctctctctccctctctccctctccctcctctctccctctccctctcctctctctcctctctctctctccctctctccctctccctctctctctctctctccctctccctctctctctcctccctcctctctctctcctctctctcctctctctccctctctctctctctctctctccctctctc

SEQ ID NO: 12 - RNA, 64 nt, synthetic construct
  source 1..64: mol_type = other RNA
ggtttttcagactcaaggagcggcagtgtcatcaatggctcaaaccctactgccggatccggtt

SEQ ID NO: 13 - DNA, 511 bp, Homo sapiens
  source 1..511: mol_type = genomic DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 14 - DNA, 39 bp, synthetic construct
  source 1..39: mol_type = other DNA
ggtaggcgtgtacggtgggaggcctatataagcagagct

(19) United States
(12) Patent Application Publication
(10) Pub. No.: US 2026/0295089 A1
(43) Pub. Date: Oct. 1, 2026

(54) Programmable miRNA-Dependent Gene Expression

(71) Applicant: New York University, New York, NY (US)

(72) Inventor: Benjamin TenOever, Scarsdale, NY (US)

(73) Assignee: New York University, New York, NY (US)

(21) Appl. No.: 19/406,799

(22) Filed: Dec. 2, 2025

Related U.S. Application Data
(63) Continuation-in-part of application No. PCT/US24/34049, filed on Jun. 14, 2024.
(60) Provisional application No. 63/521,537, filed on Jun. 16, 2023.
(60) Provisional application No. 63/726,969, filed on Dec. 2, 2024.
(60) Provisional application No. 63/787,595, filed on Apr. 11, 2025.

(51) Int. Cl. A61K 48/00; C07K 14/005; C07K 14/47; C12N 9/22; C12N 15/11; C12N 15/86

(52) U.S. Cl. CPC A61K 48/0058; C07K 14/005; C07K 14/4702; C12N 9/226; C12N 15/111; C12N 15/86; C07K 2319/95; C12N 2310/20; C12N 2740/15043; C12N 2750/14143; C12N 2760/18643; C12N 2795/00022

(57) ABSTRACT

The present invention provides miRNA-dependent polynucleotide circuits which can be programmed to express a target polypeptide in a target cell type or tissue, or a cell in a specific state, or a target species. The present invention further provides vectors, host cells, and pharmaceutical compositions comprising the miRNA-dependent polynucleotide circuits. The present invention further provides a method of selectively expressing a target polypeptide in a target cell type or tissue, or a cell in a specific state.

### CROSS-REFERENCE TO RELATED APPLICATIONS

This patent application is a continuation-in-part application of International Patent Application No. PCT/US24/34049, filed Jun. 14, 2024, which claims priority to U.S. Provisional Application No. 63/521,537, filed on Jun. 16, 2023, the disclosures of which are herein incorporated by reference in their entireties. This patent application also claims priority to U.S. Provisional Application No. 63/726,969, filed Dec. 2, 2024, and U.S. Provisional Application No. 63/787,595, filed Apr. 11, 2025, the disclosures of which are herein incorporated by reference in their entireties.

### SEQUENCE LISTING

The instant application contains a Sequence Listing which has been submitted electronically in XML format and is hereby incorporated by reference in its entirety. Said XML copy, created on Jun. 13, 2024, is named 243735_000349_SL.xml and is 11,058 bytes in size.

### FIELD OF THE INVENTION

The present invention relates to miRNA-dependent polynucleotide circuits which can be programmed to express a target polypeptide in a target cell type or tissue, or a cell in a specific state, or a target species. The present invention further relates to vectors, host cells, and pharmaceutical compositions comprising the miRNA-dependent polynucleotide circuits. The present invention further relates to a method of selectively expressing a target polypeptide in a target cell type or tissue, or a cell in a specific state.

### BACKGROUND

The capacity to deliver a desired biologic cargo to a specific cell type or tissue is arguably the most significant barrier in transforming basic science discoveries into clinically relevant therapeutics. Experimental platforms presently used to mediate delivery generally focus on virus-dependent and -independent carriers and attempt to achieve specificity in a variety of ways. Despite some notable successes, enabling specific delivery of a desired biologic cargo remains the central roadblock for the vast majority of clinical needs¹.

Despite the excitement surrounding the use of mRNA-based therapeutics, utilization of this platform is limited to vaccine applications where short-term expression in a very small number of cells is sufficient to achieve the desired outcome². For most therapeutic needs, a platform that can elicit a more sustained response in a greater number of specific cell types is generally required. In this clinical space, the use of viral vectors to deliver therapeutic cargoes has gained significant regulatory approval in recent years³. At present, many biologic-based therapies rely on one of three viral vectors, notably, adeno-associated-virus (AAV) vectors, adenovirus vectors, or lentivirus vectors, although many alternative platforms are also in development⁴. AAV and adenovirus vectors are typically used in therapies where they are directly administered to patients by infusion, with AAV being the most popular vector for areas outside of oncology and vaccines. Lentivirus vectors are typically used for ex vivo therapies, in which cells harvested from a patient are isolated and treated in the lab prior to being re-introduced⁴. While existing viral vector-based therapies such as these continue to make significant advances, the full extent of their clinical impact remains unclear in part because of a number of limitations including achieving cell specificity. Following successful entry of a given viral vector, its therapeutic efficacy depends on the quality and regulation of transgene expression. Specifically, the biologic cargo of interest must be expressed at the appropriate level, in the appropriate cells, and for the appropriate duration to mediate the desired clinical effect. A major challenge in achieving this precision is the risk of aberrant, off-target expression, which can compromise safety and efficacy. Efforts to achieve cell-type specificity have traditionally focused on the inclusion of regulatory elements such as cell-specific promoters. However, these efforts have not yet materialized into a technology that provides programable tissue or cell-specific resolution as it relates to biologic activity, as they often face significant limitations, including constraints on cargo size, incomplete restriction of expression to desired cell populations, and a lack of dynamic control over gene expression in response to the local cellular environment.

Accordingly, there is an unmet need for new strategies to achieve programable, tissue or cell-specific delivery of biologic cargos. Such approaches would allow for precise, context-dependent regulation of therapeutic genes, ensuring that expression is tightly restricted to target cell populations while minimizing off-target effects.

### SUMMARY OF THE INVENTION

In one aspect, provided herein is a polynucleotide comprising:
a) a nucleotide sequence encoding an RNA-binding protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the RNA-binding protein to allow expression of the RNA-binding protein in the absence of the respective miRNA targeting said MSE sequence;
c) a first RNA element to which the RNA-binding protein specifically binds;
d) a nucleotide sequence encoding a first target polypeptide, wherein the first RNA element is positioned with respect to the nucleotide sequence encoding the first target polypeptide to allow translation of the first target polypeptide in either the presence or absence of the RNA-binding protein; and
e) optionally one or more additional combinations of an RNA element to which the RNA-binding protein specifically binds and a nucleotide sequence encoding a target polypeptide, wherein within each combination, the RNA element is positioned with respect to the nucleotide sequence encoding the target polypeptide to allow translation of the target polypeptide in either the presence or absence of the RNA-binding protein, wherein translation of each additional target polypeptide is controlled by its closest RNA element in the resulting mRNA.

In some embodiments, the RNA-binding protein is selected from a ribosomal frameshift stimulator, an iron regulatory protein, Lin28, MS2 bacteriophage coat protein, PP7 bacteriophage coat protein, and U1 small nuclear ribonucleoprotein A (U1A).

In some embodiments, the first RNA element is selected from a ribosomal frameshift element, an iron response element, a pre-let-7 element, an MS2 responsive RNA element, a PP7 responsive RNA element, and a U1A responsive RNA element.

In some embodiments, the first RNA element to which the RNA-binding protein specifically binds comprises at least 30 nucleotides.

In some embodiments, the RNA-binding protein is selected from a ribosomal frameshift stimulator, an iron regulatory protein, and Lin28.

In some embodiments, the first RNA element is selected from a ribosomal frameshift element, an iron response element, and a pre-let-7 element.

In various embodiments, the one or more MSE sequences are positioned in the open reading frame (ORF) or in the 3′ untranslated region (3′ UTR) of the RNA-binding protein transcript coding sequence.

In various embodiments, the polynucleotide further comprises a first promoter, and wherein expression of the RNA-binding protein is under the control of the first promoter.

In some embodiments, expression of the first target polypeptide is under the control of the first promoter.

In some embodiments, the polynucleotide is structured as an alternatively spliced construct, and one transcript comprises the nucleotide sequence encoding the RNA-binding protein and one or more MSE sequences, and another transcript comprises the first RNA element to which the RNA-binding protein specifically binds and the nucleotide sequence encoding the first target polypeptide.

In some embodiments, the polynucleotide further comprises a 5′ donor splice site and a 3′ acceptor splice site. In one embodiment, the 5′ donor splice site comprises the sequence AG*GU(A/G)AGU or AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, the polynucleotide comprises one or more additional combinations of an RNA element to which the RNA-binding protein specifically binds and a nucleotide sequence encoding a target polypeptide, and expression of the first target polypeptide and the one or more additional target polypeptides is under the control of the first promoter.

In some embodiments, the polynucleotide comprises one or more additional combinations of an RNA element to which the RNA-binding protein specifically binds and a nucleotide sequence encoding a target polypeptide, and expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of an RNA element to which the RNA-binding protein specifically binds and a nucleotide sequence encoding a target polypeptide, and wherein expression of each of the additional target polypeptides is under the control of respective additional promoters.

In some embodiments, the polynucleotide further comprises one or more DNA insulator elements, wherein said one or more DNA insulator elements are positioned between the 3′UTR of the RNA-binding protein transcript coding sequence and the second promoter, and wherein said DNA one or more DNA insulator elements enhance efficiency of termination of transcription of the RNA-binding protein.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, the DNA insulator element comprises CCCTC-binding factor site(s).

In various embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In various embodiments, the second promoter is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of the RNA-binding protein in one direction and the first target polypeptide in the opposite direction. In some embodiments, bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In some embodiments, the nucleotide sequence encoding the RNA-binding protein further encodes a degron sequence. In some embodiments, the degron sequence is operably linked to the C-terminal end of the RNA-binding protein in the resulting protein. In some embodiments, the degron sequence is operably linked to the N-terminal end of the RNA-binding protein in the resulting protein. In some embodiments, the degron sequence comprises the amino acid sequence of SEQ ID NO: 17, or a functional variant or ortholog having at least 70% sequence identity thereto.

In another aspect, provided herein is a polynucleotide comprising:
a) a nucleotide sequence encoding a ribosomal frameshift stimulator;
b) one or more microRNA (miRNA) Silencing Element (MSE) sequences, wherein each of the one or more MSE sequences is targeted by a miRNA, and wherein each of the one or more MSE sequences is positioned with respect to the nucleotide sequence encoding the ribosomal frameshift stimulator to allow expression of the ribosomal frameshift stimulator in the absence of the respective miRNA targeting the MSE sequence;
c) a first ribosomal frameshift element;
d) a nucleotide sequence encoding a first target polypeptide,
   wherein the first ribosomal frameshift element is positioned 5′ to the nucleotide sequence encoding the first target polypeptide, and
   wherein translation of the first target polypeptide is controlled by the first ribosomal frameshift element and requires either the presence or absence of the ribosomal frameshift stimulator, and
e) optionally one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

In some embodiments, the one or more MSE sequences are positioned in the open reading frame (ORF) or in the 3′ untranslated region (3′ UTR) of the ribosomal frameshift stimulator transcript coding sequence.

In some embodiments, the polynucleotide further comprises a first promoter, and wherein expression of the ribosomal frameshift stimulator is under the control of the first promoter.

In some embodiments, expression of the first target polypeptide is under the control of the first promoter.

In some embodiments, the polynucleotide is structured as an alternatively spliced construct, wherein one transcript comprises the nucleotide sequence encoding the ribosomal frameshift stimulator and one or more MSE sequences, and another transcript comprises the first ribosomal frameshift element and the nucleotide sequence encoding the first target polypeptide. In some embodiments, the polynucleotide is structured as an alternatively spliced construct, wherein one transcript comprises the nucleotide sequence encoding the ribosomal frameshift stimulator and one or more MSE sequences, and another transcript does not comprise the nucleotide sequence encoding the ribosomal frameshift stimulator and one or more MSE sequence. In some embodiments, the polynucleotide further comprises a 5′ donor splice site and a 3′ acceptor splice site. In one embodiment, the 5′ donor splice site comprises the sequence AG*GU(A/G)AGU or AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, the polynucleotide comprises one or more additional combinations of a ribosomal frameshift element and a nucleotide sequence encoding a target polypeptide, and wherein expression of the first target polypeptide and the one or more additional target polypeptides is under the control of the first promoter.

In some embodiments, the polynucleotide comprises one or more additional combinations of a ribosomal frameshift element and a nucleotide sequence encoding a target polypeptide, and wherein expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of a ribosomal frameshift element and a nucleotide sequence encoding a target polypeptide, and wherein expression of each of the additional target polypeptides is under the control of respective additional promoters.

In some embodiments, the polynucleotide further comprises one or more DNA insulator elements, wherein the one or more DNA insulator elements are positioned between the 3′UTR of the ribosomal frameshift stimulator transcript coding sequence and the second promoter, and wherein the DNA one or more DNA insulator elements enhance efficiency of termination of transcription of the ribosomal frameshift stimulator.

In some embodiments, the polynucleotide further comprises one or more DNA insulator elements, wherein the DNA insulator elements are positioned between the 3′UTR of each subsequent transcript and each subsequent promoter, and wherein the DNA insulator elements enhance efficiency of termination of transcription. In one embodiment, the DNA insulator element comprises CCCTC-binding factor site(s).

In some embodiments, the nucleotide sequence encoding the first target polypeptide is positioned in frame with the first ribosomal frameshift element.

In some embodiments, the nucleotide sequence encoding the first target polypeptide is positioned in-1 translation frame in relation to the first ribosomal frameshift element.

In some embodiments, the polynucleotide comprises one or more additional combinations of a ribosomal frameshift element and a nucleotide sequence encoding a target polypeptide, and wherein the nucleotide sequence encoding each additional target polypeptide is independently positioned in frame or in-1 translation frame in relation to its closest 5′ ribosomal frameshift element in the resulting mRNA.

In one embodiment, provided herein is a polynucleotide comprising from 5′ to 3′:
a) a first promoter;
b) a 5′ donor splice site;
c) a nucleotide sequence encoding a ribosomal frameshift stimulator;
d) one or more microRNA (miRNA) Silencing Element (MSE) sequences, wherein each of the one or more MSE sequences is targeted by a miRNA, and wherein each of the one or more MSE sequences is positioned with respect to the nucleotide sequence encoding the ribosomal frameshift stimulator to allow expression of the ribosomal frameshift stimulator in the absence of the respective miRNA targeting the MSE sequence;
e) a 3′ splice acceptor site,
f) a first ribosomal frameshift element, and
g) a nucleotide sequence encoding a first target polypeptide,
   wherein the first ribosomal frameshift element is positioned 5′ to the nucleotide sequence encoding the first target polypeptide,
   wherein translation of the first target polypeptide is controlled by the first ribosomal frameshift element and requires either the presence or absence of the ribosomal frameshift stimulator, and
   wherein the nucleotide sequence encoding the first target polypeptide is positioned in frame or in-1 translation frame in relation to the first ribosomal frameshift element.

In one embodiment, the polynucleotide described above further comprises 3′ to the nucleotide sequence encoding the first target polypeptide from 5′ to 3′:
h) a second promoter;
i) a second ribosomal frameshift element, and
j) a nucleotide sequence encoding a second target polypeptide,
   wherein the second ribosomal frameshift element is positioned 5′ to the nucleotide sequence encoding the second target polypeptide,
   wherein translation of the second target polypeptide is controlled by the second ribosomal frameshift element and requires either the presence or absence of the ribosomal frameshift stimulator, and
   wherein the nucleotide sequence encoding the second target polypeptide is positioned in frame or in-1 translation frame in relation to the second ribosomal frameshift element.

In one embodiment, the polynucleotide further comprises a DNA insulator element, wherein the DNA insulator element is positioned between the nucleotide sequence encoding the first target polypeptide and the second promoter, and wherein the DNA insulator element enhances efficiency of termination of transcription.

In one embodiment, the 5′ donor splice site comprises the sequence AG*GU(A/G)AGU or AG*GU(A/G)GAU, wherein * denotes the splice junction.

In one embodiment, the 3′ acceptor splice site comprises a branch point, a pyrimidine tract, and the sequence AG*G, wherein * denotes the splice junction.

In one embodiment, provided herein is a polynucleotide comprising from 5′ to 3′:
a) a first promoter;
b) a nucleotide sequence encoding a ribosomal frameshift stimulator;
c) one or more microRNA (miRNA) Silencing Element (MSE) sequences, wherein each of the one or more MSE sequences is targeted by a miRNA, and wherein each of the one or more MSE sequences is positioned with respect to the nucleotide sequence encoding the ribosomal frameshift stimulator to allow expression of the ribosomal frameshift stimulator in the absence of the respective miRNA targeting the MSE sequence,
d) a second promoter;
e) a first ribosomal frameshift element, and
f) a nucleotide sequence encoding a first target polypeptide,
   wherein the first ribosomal frameshift element is positioned 5′ to the nucleotide sequence encoding the first target polypeptide,
   wherein translation of the first target polypeptide is controlled by the first ribosomal frameshift element and requires either the presence or absence of the ribosomal frameshift stimulator, and
   wherein the nucleotide sequence encoding the first target polypeptide is positioned in frame or in-1 translation frame in relation to the first ribosomal frameshift element.

In one embodiment, the polynucleotide further comprises a DNA insulator element, wherein the DNA insulator element is positioned between the 3′UTR of the ribosomal frameshift stimulator transcript coding sequence and the second promoter, and wherein the DNA insulator element enhances efficiency of termination of transcription.

In one embodiment, the polynucleotide further comprises 3′ to the nucleotide sequence encoding the first target polypeptide from 5′ to 3′:
g) a third promoter;
h) a second ribosomal frameshift element, and
i) a nucleotide sequence encoding a second target polypeptide,
   wherein the second ribosomal frameshift element is positioned 5′ to the nucleotide sequence encoding the second target polypeptide,
   wherein translation of the second target polypeptide is controlled by the second ribosomal frameshift element and requires either the presence or absence of the ribosomal frameshift stimulator, and
   wherein the nucleotide sequence encoding the second target polypeptide is positioned in frame or in-1 translation frame in relation to the second ribosomal frameshift element.

In one embodiment, the polynucleotide further comprises a DNA insulator element, wherein the DNA insulator element is positioned between the nucleotide sequence encoding the first target polypeptide and the third promoter, and wherein the DNA insulator element enhances efficiency of termination of transcription.

In some embodiments, the DNA insulator element described herein comprises CCCTC-binding factor site(s).

In various embodiments, the first promotor is an RNA polymerase II promoter. The first promoter may be a cytomegalovirus (CMV) promoter, a simian virus 40 (SV40) promoter, a human elongation factor 1 alpha (EF1α) promoter, or a phosphoglycerate kinase (PGK) promoter.

In various embodiments, the second promoter is an RNA polymerase II promoter. The second promoter may be a cytomegalovirus (CMV) promoter, a simian virus 40 (SV40) promoter, a human elongation factor 1 alpha (EF1α) promoter, or a phosphoglycerate kinase (PGK) promoter.

In various embodiments, the third promoter is an RNA polymerase II promoter. The third promoter is a cytomegalovirus (CMV) promoter, a simian virus 40 (SV40) promoter, a human elongation factor 1 alpha (EF1α) promoter, or a phosphoglycerate kinase (PGK) promoter.

In various embodiments, the first promoter is a bi-directional promoter which drives expression of the ribosomal frameshift stimulator in one direction and the first target polypeptide in the opposite direction.

In another aspect, provided herein is a polynucleotide comprising from 5′ to 3′:
a) one or more microRNA (miRNA) Silencing Element (MSE) sequences, wherein each of said one or more MSE sequences is targeted by a miRNA,
b) a nucleotide sequence encoding a ribosomal frameshift stimulator, wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
c) a bi-directional promoter;
d) a first ribosomal frameshift element;
e) a nucleotide sequence encoding a first target polypeptide, wherein the first ribosomal frameshift element is positioned 5′ to the nucleotide sequence encoding the first target polypeptide, wherein translation of the first target polypeptide is controlled by the first ribosomal frameshift element and requires either the presence or absence of the ribosomal frameshift stimulator, and wherein the bi-directional promoter drives expression of the ribosomal frameshift stimulator in one direction and the first target polypeptide in the opposite direction; and
f) optionally one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

In various embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In various embodiments, the ribosomal frameshift stimulator is a cardiovirus 2A protein. In one embodiment, the cardiovirus 2A protein comprises the amino acid sequence SEQ ID NO: 1, or a variant thereof. In one embodiment, the cardiovirus 2A protein consists of the amino acid sequence SEQ ID NO: 1, or a variant thereof. In one embodiment, the nucleotide sequence encoding the cardiovirus 2A protein comprises the sequence SEQ ID NO: 2, or a variant thereof. In one embodiment, the nucleotide sequence encoding the cardiovirus 2A protein consists of the sequence SEQ ID NO: 2, or a variant thereof.

In various embodiments, the first ribosomal frameshift element comprises from 5′ to 3′:
1) a 5′ shift site,
2) a spacer sequence, and
3) a stem-loop structure.

In various embodiments, the second ribosomal frameshift element comprises from 5′ to 3′:
1) a 5′ shift site,
2) a spacer sequence, and
3) a stem-loop structure.

In one embodiment, the 5′ shift site comprises the sequence 5′-GGUUUUU-3′. In one embodiment, the 5′ shift site consists of the sequence 5′-GGUUUUU-3′.

In some embodiments, the spacer is 13 or 14 nucleotides long. In one embodiment, the spacer comprises the sequence SEQ ID NO: 4. In one embodiment, the spacer consists of the sequence SEQ ID NO: 4.

In some embodiments, the stem-loop structure comprises a stem of 7 nucleotides. In one embodiment, the stem-loop structure comprises the sequence 5′-CGGCAGU-3′ followed by a 21-nucleotide loop and the complementary sequence 5′ACUGCCG-3′.

In some embodiments, the loop of the stem-loop structure comprises an adenosine triplet followed by a cytosine triplet. In one embodiment, the loop structure comprises the sequence GUCAUCAAUGGCUCAAACCCU (SEQ ID NO: 7). In one embodiment, the loop structure consists of the sequence GUCAUCAAUGGCUCAAACCCU (SEQ ID NO: 7).

In various embodiments, the first ribosomal frameshift element comprises the sequence
GGUUUUUCAGACUCAAGGAGCGGCAGUGUCAUCAAUGGCUCAAACCCUACUGC CG (SEQ ID NO: 8), or a sequence having at least 60% identity thereto.

In various embodiments, the second ribosomal frameshift element comprises the sequence
GGUUUUUCAGACUCAAGGAGCGGCAGUGUCAUCAAUGGCUCAAACCCUACUGC CG (SEQ ID NO: 8), or a sequence having at least 60% identity thereto.

In various embodiments, the first ribosomal frameshift element causes −1 frameshift in the presence of the ribosomal frameshift stimulator.

In various embodiments, the second ribosomal frameshift element causes a −1 frameshift in the presence of the ribosomal frameshift stimulator.

In various embodiments, the polynucleotide further comprises one or more additional combinations of an additional ribosomal frameshift element and a nucleotide sequence encoding an additional target polypeptide, wherein the additional ribosomal frameshift element is positioned 5′ to the nucleotide sequence encoding the additional target polypeptide, wherein translation of the additional target polypeptide is controlled by the additional ribosomal frameshift element and requires either the presence or absence of the ribosomal frameshift stimulator, and wherein the nucleotide sequence encoding the additional target polypeptide is positioned in frame or in −1 translation frame in relation to the additional ribosomal frameshift element.

In another aspect, provided herein is a polynucleotide comprising:
a) a nucleotide sequence encoding an iron regulatory protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence;
c) a first iron response element;
d) a nucleotide sequence encoding a first target polypeptide, wherein the first iron response element is positioned with respect to the nucleotide sequence encoding the first target polypeptide to allow translation of the first target polypeptide in the absence of the iron regulatory protein; and
e) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In some embodiments, the one or more MSE sequences are positioned in the open reading frame (ORF) or in the 3′ untranslated region (3′ UTR) of the iron regulatory protein transcript coding sequence.

In some embodiments, the polynucleotide further comprises a first promoter, and wherein expression of the iron regulatory protein is under the control of the first promoter.

In some embodiments, expression of the first target polypeptide is under the control of the first promoter.

In some embodiments, the polynucleotide is structured as an alternatively spliced construct, and wherein one transcript comprises the nucleotide sequence encoding the iron regulatory protein and one or more MSE sequences, and another transcript comprises the first iron response element and the nucleotide sequence encoding the first target polypeptide.

In some embodiments, the polynucleotide further comprises a 5′ donor splice site and a 3′ acceptor splice site. In one embodiment, the 5′ donor splice site comprises the sequence AG*GU(A/G)AGU or AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, the polynucleotide comprises one or more additional combinations of an iron response element and a nucleotide sequence encoding a target polypeptide, and expression of the first target polypeptide and the one or more additional target polypeptides is under the control of the first promoter.

In some embodiments, the polynucleotide comprises one or more additional combinations of an iron response element and a nucleotide sequence encoding a target polypeptide, and expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of an iron response element and a nucleotide sequence encoding a target polypeptide, and wherein expression of each of the additional target polypeptides is under the control of respective additional promoters.

In some embodiments, the polynucleotide further comprises one or more DNA insulator elements, wherein said one or more DNA insulator elements are positioned between the 3′UTR of the iron regulatory protein transcript coding sequence and the second promoter, and wherein said DNA one or more DNA insulator elements enhance efficiency of termination of transcription of the iron regulatory protein.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, the DNA insulator element comprises CCCTC-binding factor site(s).

In one embodiment, provided herein is a polynucleotide comprising from 5′ to 3′:
a) a first promoter;
b) a 5′ donor splice site;
c) a nucleotide sequence encoding an iron regulatory protein;
d) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence;
e) a 3′ splice acceptor site;
f) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
g) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In one embodiment, the 5′ donor splice site comprises the sequence AG*GU(A/G)AGU or AG*GU(A/G)GAU, wherein * denotes the splice junction.

In one embodiment, the 3′ acceptor splice site comprises a branch point, a pyrimidine tract, and the sequence AG*G, wherein * denotes the splice junction.

In one embodiment, provided herein is a polynucleotide comprising from 5′ to 3′:
a) a first promoter;
b) a nucleotide sequence encoding an iron regulatory protein;
c) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence,
d) a second promoter;
e) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
f) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In one embodiment, the polynucleotide further comprises a DNA insulator element, wherein said DNA insulator element is positioned between the 3′UTR of the iron regulatory protein transcript coding sequence and the second promoter, and wherein said DNA insulator element enhances efficiency of termination of transcription. In one embodiment, the DNA insulator element comprises CCCTC-binding factor site(s).

In various embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In various embodiments, the second promotor is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of the iron regulatory protein in one direction and the first target polypeptide in the opposite direction.

In one embodiment, provided herein is a polynucleotide comprising from 5′ to 3′:
a) one or more microRNA (miRNA) Silencing Element (MSE) sequences, wherein each of said one or more MSE sequences is targeted by a miRNA;
b) a nucleotide sequence encoding an iron regulatory protein, wherein each of said one or more MSE sequences is positioned with respect to the nucleotide sequence encoding the iron regulatory protein to allow expression of the iron regulatory protein in the absence of the respective miRNA targeting said MSE sequence;
c) a bi-directional promoter;
d) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
e) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In some embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In various embodiments, the iron regulatory protein is Iron Regulatory Protein 2 (IRP2), Iron Regulatory Protein 1 (IRP1), or a functional variant or ortholog thereof.

In some embodiments, the iron regulatory protein is human IRP2, or a functional variant or ortholog thereof.

In some embodiments, the human IRP2 comprises the amino acid sequence of SEQ ID NO: 16, or a sequence having at least 70% sequence identity thereto.

In some embodiments, the human IRP2 variant comprises the amino acid sequence of any one of SEQ ID NOs: 21, 24, 25, 26, 27, and 30, or a sequence having at least 70% sequence identity thereto.

In some embodiments, the human IRP2 variant comprises or consists essentially of the amino acids 1-318 and 443-750 of SEQ ID NO: 16.

In some embodiments, the human IRP2 variant comprises a substitution at one or more positions selected from cysteine 120, cysteine 375, cysteine 578, and cysteine 581.

In some embodiments, the iron regulatory protein is human IRP1, or a functional variant or ortholog thereof. In some embodiments, the human IRP1 comprises the amino acid sequence of SEQ ID NO: 15, or a sequence having at least 70% sequence identity thereto.

In some embodiments, the nucleotide sequence encoding an iron regulatory protein further encodes a degron sequence. In some embodiments, the degron sequence is operably linked to the C-terminal end of the iron regulatory protein in the resulting protein. In some embodiments, the degron sequence is operably linked to the N-terminal end of the iron regulatory protein in the resulting protein. In some embodiments, the degron sequence comprises the amino acid sequence of SEQ ID NO: 17, or a functional variant or ortholog having at least 70% sequence identity thereto.

In various embodiments, the first iron response element is positioned in the 5′ UTR or the 3′ UTR of the nucleotide sequence encoding a first target polypeptide.

In various embodiments, when one or more additional combinations of an iron response element and a nucleotide sequence encoding a target polypeptide are present, each additional iron response element is positioned independently in the 5′ UTR or the 3′ UTR of the nucleotide sequence encoding the respective additional target polypeptide.

In various embodiments, the iron response element comprises a stem-loop element comprising a 6-nucleotide apical loop motif 5′-CAGWGH-3′ (W=A or U and H=A, C, or U) operably linked to a five base-pair stem.

In some embodiments, the iron response element further comprises an internal bulge containing a conserved cytosine (C) operably linked to the five base-pair stem.

In some embodiments, the iron response element comprises an iron response element found in the 5′ UTR of the human ferritin transcript, hypoxia inducible factor 1 subunit alpha (HIF1A) transcript, or erythroid 5-aminolevulinate synthase (eALAS) transcript, or a functional variant or ortholog thereof.

In some embodiments, the iron response element comprises the nucleotide sequence of any one of SEQ ID Nos: 32, 33, or 34, or a sequence having at least 70% sequence identity thereto.

In another aspect, provided herein is a polynucleotide comprising:
a) a nucleotide sequence encoding Lin28;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding Lin28 to allow expression of Lin28 in the absence of the respective miRNA targeting said MSE sequence;
c) a first pre-let-7 element;
d) a nucleotide sequence encoding a first target polypeptide, wherein the first pre-let-7 element is positioned with respect to the nucleotide sequence encoding the first target polypeptide to allow translation of the first target polypeptide in the absence of Lin28; and
e) optionally one or more additional combinations of a pre-let-7 element and a nucleotide sequence encoding a target polypeptide, wherein within each combination, the pre-let-7 element is positioned with respect to the nucleotide sequence encoding the target polypeptide to allow translation of the target polypeptide in the absence of Lin28, wherein translation of each additional target polypeptide is controlled by its closest pre-let-7 element in the resulting mRNA.

In some embodiments, the one or more MSE sequences are positioned in the open reading frame (ORF) or in the 3′ untranslated region (3′ UTR) of the Lin28 transcript coding sequence.

In some embodiments, the polynucleotide further comprises a first promoter, and wherein expression of Lin28 is under the control of the first promoter.

In some embodiments, expression of the first target polypeptide is under the control of the first promoter.

In some embodiments, the polynucleotide is structured as an alternatively spliced construct, and wherein one transcript comprises the nucleotide sequence encoding Lin28 and one or more MSE sequences, and another transcript comprises the first pre-let-7 element and the nucleotide sequence encoding the first target polypeptide.

In some embodiments, the polynucleotide further comprises a 5′ donor splice site and a 3′ acceptor splice site. In one embodiment, the 5′ donor splice site comprises the sequence AG*GU(A/G)AGU or AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, the polynucleotide comprises one or more additional combinations of a pre-let-7 element and a nucleotide sequence encoding a target polypeptide, and wherein expression of the first target polypeptide and the one or more additional target polypeptides is under the control of the first promoter.

In some embodiments, the polynucleotide comprises one or more additional combinations of a pre-let-7 element and a nucleotide sequence encoding a target polypeptide, and wherein expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of a pre-let-7 element and a nucleotide sequence encoding a target polypeptide, and wherein expression of each of the additional target polypeptides is under the control of respective additional promoters.

In some embodiments, the polynucleotide further comprises one or more DNA insulator elements, wherein said one or more DNA insulator elements are positioned between the 3′UTR of Lin28 transcript coding sequence and the second promoter, and wherein said DNA one or more DNA insulator elements enhance efficiency of termination of transcription of the Lin28.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, the DNA insulator element comprises CCCTC-binding factor site(s).

In various embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In various embodiments, the second promotor is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of Lin28 in one direction and the first target polypeptide in the opposite direction.

In some embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In some embodiments, Lin28 comprises the amino acid sequence of SEQ ID NO: 35, or a sequence having at least 70% sequence identity thereto.

In some embodiments, the nucleotide sequence encoding Lin28 further encodes a degron sequence. In some embodiments, the degron sequence is operably linked to the C-terminal end of Lin28 in the resulting protein. In some embodiments, the degron sequence is operably linked to the N-terminal end of Lin28 in the resulting protein. In some embodiments, the degron sequence comprises the amino acid sequence of SEQ ID NO: 17, or a functional variant or ortholog having at least 70% sequence identity thereto.

In some embodiments, the first pre-let-7 element is positioned in the 5′ UTR or the 3′ UTR of the nucleotide sequence encoding a first target polypeptide.

In some embodiments, when one or more additional combinations of a pre-let-7 element and a nucleotide sequence encoding a target polypeptide are present, each additional pre-let-7 element is positioned independently in the 5′ UTR or the 3′ UTR of the nucleotide sequence encoding the respective additional target polypeptide.

In some embodiments, the pre-let-7 element comprises a tetranucleotide motif 5′-GGAG-3′.

In some embodiments, the pre-let-7 element comprises the nucleotide sequence of SEQ ID No: 36, or a sequence having at least 70% sequence identity thereto.

In another aspect, provided herein is a polynucleotide comprising:
a) a nucleotide sequence encoding an MS2 bacteriophage coat protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the MS2 bacteriophage coat protein to allow expression of the MS2 bacteriophage coat protein in the absence of the respective miRNA targeting said MSE sequence;
c) a first MS2 responsive RNA element;
d) a nucleotide sequence encoding a first target polypeptide, wherein the first MS2 responsive RNA element is positioned with respect to the nucleotide sequence encoding the first target polypeptide to allow translation of the first target polypeptide in the absence of the MS2 bacteriophage coat protein; and
e) optionally one or more additional combinations of an MS2 responsive RNA element and a nucleotide sequence encoding a target polypeptide, wherein within each combination, the MS2 responsive RNA element is positioned with respect to the nucleotide sequence encoding the target polypeptide to allow translation of the target polypeptide in the absence of the MS2 bacteriophage coat protein, wherein translation of each additional target polypeptide is controlled by its closest MS2 responsive RNA element in the resulting mRNA.

In some embodiments, the one or more MSE sequences are positioned in the open reading frame (ORF) or in the 3′ untranslated region (3′ UTR) of the MS2 transcript coding sequence.

In some embodiments, the polynucleotide further comprises a first promoter, and wherein expression of the MS2 bacteriophage coat protein is under the control of the first promoter.

In some embodiments, expression of the first target polypeptide is under the control of the first promoter.

In some embodiments, the polynucleotide is structured as an alternatively spliced construct, and wherein one transcript comprises the nucleotide sequence encoding the MS2 bacteriophage coat protein and one or more MSE sequences, and another transcript comprises the first MS2 responsive RNA element and the nucleotide sequence encoding the first target polypeptide.

In some embodiments, the polynucleotide further comprises a 5′ donor splice site and a 3′ acceptor splice site. In some embodiments, the 5′ donor splice site comprises the sequence AG*GU(A/G)AGU or AG*GU(A/G)GAU, wherein * denotes the splice junction. In some embodiments, the 3′ acceptor splice site comprises a branch point, a pyrimidine tract, and the sequence AG*G, wherein * denotes the splice junction.

In some embodiments, the polynucleotide comprises one or more additional combinations of an MS2 responsive RNA element and a nucleotide sequence encoding a target polypeptide, and expression of the first target polypeptide and the one or more additional target polypeptides is under the control of the first promoter.

In some embodiments, the polynucleotide comprises one or more additional combinations of an MS2 responsive RNA element and a nucleotide sequence encoding a target polypeptide, and expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of an MS2 responsive RNA element and a nucleotide sequence encoding a target polypeptide, and expression of each of the additional target polypeptides is under the control of respective additional promoters.

In some embodiments, the polynucleotide further comprises one or more DNA insulator elements, wherein said one or more DNA insulator elements are positioned between the 3′UTR of the MS2 bacteriophage coat protein transcript coding sequence and the second promoter, and wherein said DNA one or more DNA insulator elements enhance efficiency of termination of transcription of the MS2 bacteriophage coat protein.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, said DNA insulator element comprises CCCTC-binding factor site(s).

In some embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the second promoter is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of the MS2 bacteriophage coat protein in one direction and the first target polypeptide in the opposite direction. In some embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In various embodiments, the MS2 bacteriophage coat protein comprises the amino acid sequence of SEQ ID NO: 37, or a sequence having at least 70% sequence identity thereto.

In some embodiments, the nucleotide sequence encoding the MS2 bacteriophage coat protein further encodes a degron sequence. In some embodiments, the degron sequence is operably linked to the C-terminal end of the MS2 bacteriophage coat protein in the resulting protein. In some embodiments, the degron sequence is operably linked to the N-terminal end of the MS2 bacteriophage coat protein in the resulting protein.

In some embodiments, the degron sequence comprises the amino acid sequence of SEQ ID NO: 17, or a functional variant or ortholog having at least 70% sequence identity thereto.

In some embodiments, the first MS2 responsive RNA element is positioned in the 5′ UTR or the 3′ UTR of the nucleotide sequence encoding a first target polypeptide.

In some embodiments, when one or more additional combinations of a MS2 responsive RNA element and a nucleotide sequence encoding a target polypeptide are present, each additional MS2 responsive RNA element is positioned independently in the 5′ UTR or the 3′ UTR of the nucleotide sequence encoding the respective additional target polypeptide.

In various embodiments, the MS2 responsive RNA element comprises the nucleotide sequence of SEQ ID No: 38, or a sequence having at least 70% sequence identity thereto.

In another aspect, provided herein is a polynucleotide comprising:
a) a nucleotide sequence encoding a PP7 bacteriophage coat protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the PP7 bacteriophage coat protein to allow expression of the PP7 bacteriophage coat protein in the absence of the respective miRNA targeting said MSE sequence;
c) a first PP7 responsive RNA element;
d) a nucleotide sequence encoding a first target polypeptide, wherein the first PP7 responsive RNA element is positioned with respect to the nucleotide sequence encoding the first target polypeptide to allow translation of the first target polypeptide in the absence of the PP7 bacteriophage coat protein; and
e) optionally one or more additional combinations of a PP7 responsive RNA element and a nucleotide sequence encoding a target polypeptide, wherein within each combination, the PP7 responsive RNA element is positioned with respect to the nucleotide sequence encoding the target polypeptide to allow translation of the target polypeptide in the absence of the PP7 bacteriophage coat protein, wherein translation of each additional target polypeptide is controlled by its closest PP7 responsive RNA element in the resulting mRNA.

In some embodiments, the one or more MSE sequences are positioned in the open reading frame (ORF) or in the 3′ untranslated region (3′ UTR) of the PP7 transcript coding sequence.

In some embodiments, the polynucleotide further comprises a first promoter, and wherein expression of the PP7 bacteriophage coat protein is under the control of the first promoter.

In some embodiments, expression of the first target polypeptide is under the control of the first promoter.

In some embodiments, the polynucleotide is structured as an alternatively spliced construct, and wherein one transcript comprises the nucleotide sequence encoding the PP7 bacteriophage coat protein and one or more MSE sequences, and another transcript comprises the first PP7 responsive RNA element and the nucleotide sequence encoding the first target polypeptide.

In some embodiments, the polynucleotide further comprises a 5′ donor splice site and a 3′ acceptor splice site. In some embodiments, the 5′ donor splice site comprises the sequence AG*GU(A/G)AGU or AG*GU(A/G)GAU, wherein * denotes the splice junction. In some embodiments, the 3′ acceptor splice site comprises a branch point, a pyrimidine tract, and the sequence AG*G, wherein * denotes the splice junction.

In some embodiments, the polynucleotide comprises one or more additional combinations of a PP7 responsive RNA element and a nucleotide sequence encoding a target polypeptide, and wherein expression of the first target polypeptide and the one or more additional target polypeptides is under the control of the first promoter.

In some embodiments, the polynucleotide comprises one or more additional combinations of a PP7 responsive RNA element and a nucleotide sequence encoding a target polypeptide, and wherein expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of a PP7 responsive RNA element and a nucleotide sequence encoding a target polypeptide, and wherein expression of each of the additional target polypeptides is under the control of respective additional promoters.

In some embodiments, the polynucleotide further comprises one or more DNA insulator elements, wherein said one or more DNA insulator elements are positioned between the 3′UTR of the PP7 bacteriophage coat protein transcript coding sequence and the second promoter, and wherein said DNA one or more DNA insulator elements enhance efficiency of termination of transcription of the PP7 bacteriophage coat protein.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, the DNA insulator element comprises CCCTC-binding factor site(s).

In some embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the second promoter is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of the PP7 bacteriophage coat protein in one direction and the first target polypeptide in the opposite direction. In some embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In some embodiments, the PP7 bacteriophage coat protein comprises the amino acid sequence of SEQ ID NO: 39, or a sequence having at least 70% sequence identity thereto.

In some embodiments, the nucleotide sequence encoding the PP7 bacteriophage coat protein further encodes a degron sequence. In some embodiments, the degron sequence is operably linked to the C-terminal end of the PP7 bacteriophage coat protein in the resulting protein. In some embodiments, the degron sequence is operably linked to the N-terminal end of the PP7 bacteriophage coat protein in the resulting protein. In some embodiments, the degron sequence comprises the amino acid sequence of SEQ ID NO: 17, or a functional variant or ortholog having at least 70% sequence identity thereto.

In some embodiments, the first PP7 responsive RNA element is positioned in the 5′ UTR or the 3′ UTR of the nucleotide sequence encoding a first target polypeptide.

In some embodiments, when one or more additional combinations of a PP7 responsive RNA element and a nucleotide sequence encoding a target polypeptide are present, each additional PP7 responsive RNA element is positioned independently in the 5′ UTR or the 3′ UTR of the nucleotide sequence encoding the respective additional target polypeptide.

In some embodiments, the PP7 responsive RNA element comprises the nucleotide sequence of SEQ ID No: 40, or a sequence having at least 70% sequence identity thereto.

In another aspect, provided herein is a polynucleotide comprising:
a) a nucleotide sequence encoding a U1 small nuclear ribonucleoprotein A (U1A);
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the U1A to allow expression of the U1A in the absence of the respective miRNA targeting said MSE sequence;
c) a first U1A responsive RNA element;
d) a nucleotide sequence encoding a first target polypeptide, wherein the first U1A responsive RNA element is positioned with respect to the nucleotide sequence encoding the first target polypeptide to allow translation of the first target polypeptide in the absence of the U1A; and
e) optionally one or more additional combinations of a U1A responsive RNA element and a nucleotide sequence encoding a target polypeptide, wherein within each combination, the U1A responsive RNA element is positioned with respect to the nucleotide sequence encoding the target polypeptide to allow translation of the target polypeptide in the absence of the U1A, wherein translation of each additional target polypeptide is controlled by its closest U1A responsive RNA element in the resulting mRNA.

In some embodiments, the one or more MSE sequences are positioned in the open reading frame (ORF) or in the 3′ untranslated region (3′ UTR) of the U1A transcript coding sequence.

In some embodiments, the polynucleotide further comprises a first promoter, and wherein expression of the U1A is under the control of the first promoter.

In some embodiments, expression of the first target polypeptide is under the control of the first promoter.

In some embodiments, the polynucleotide is structured as an alternatively spliced construct, and wherein one transcript comprises the nucleotide sequence encoding the U1A and one or more MSE sequences, and another transcript comprises the first U1A responsive RNA element and the nucleotide sequence encoding the first target polypeptide.

In some embodiments, the polynucleotide further comprises a 5′ donor splice site and a 3′ acceptor splice site. In some embodiments, the 5′ donor splice site comprises the sequence AG*GU(A/G)AGU or AG*GU(A/G)GAU, wherein * denotes the splice junction. In some embodiments, the 3′ acceptor splice site comprises a branch point, a pyrimidine tract, and the sequence AG*G, wherein * denotes the splice junction.

In some embodiments, the polynucleotide comprises one or more additional combinations of a U1A responsive RNA element and a nucleotide sequence encoding a target polypeptide, and wherein expression of the first target polypeptide and the one or more additional target polypeptides is under the control of the first promoter.

In some embodiments, the polynucleotide comprises one or more additional combinations of a U1A responsive RNA element and a nucleotide sequence encoding a target polypeptide, and wherein expression of the first target polypeptide is under the control of the first promoter, and expression of each of the additional target polypeptides is under the control of respective additional promoters.

In some embodiments, the polynucleotide further comprises a second promoter, wherein expression of the first target polypeptide is under the control of the second promoter.

In some embodiments, the polynucleotide comprises one or more additional combinations of a U1A responsive RNA element and a nucleotide sequence encoding a target polypeptide, and wherein expression of each of the additional target polypeptides is under the control of respective additional promoters.

In some embodiments, the polynucleotide further comprises one or more DNA insulator elements, wherein said one or more DNA insulator elements are positioned between the 3′UTR of the U1A transcript coding sequence and the second promoter, and wherein said DNA one or more DNA insulator elements enhance efficiency of termination of transcription of the U1A.

In some embodiments, the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

In some embodiments, the DNA insulator element comprises CCCTC-binding factor site(s).

In some embodiments, the first promotor is an RNA polymerase II promoter. In some embodiments, the first promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the second promoter is an RNA polymerase II promoter. In some embodiments, the second promoter is a cytomegalovirus (CMV) promoter, a simian virus 40 (SV40) promoter, a human elongation factor 1 alpha (EF1α) promoter, or a phosphoglycerate kinase (PGK) promoter.

In some embodiments, the first promoter is a bi-directional promoter which drives expression of the U1A in one direction and the first target polypeptide in the opposite direction. In some embodiments, the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

In various embodiments, the U1A comprises the amino acid sequence of SEQ ID NO: 41, or a sequence having at least 70% sequence identity thereto.

In some embodiments, the nucleotide sequence encoding the U1A further encodes a degron sequence. In some embodiments, the degron sequence is operably linked to the C-terminal end of the U1A in the resulting protein. In some embodiments, the degron sequence is operably linked to the N-terminal end of the U1A in the resulting protein. In some embodiments, the degron sequence comprises the amino acid sequence of SEQ ID NO: 17, or a functional variant or ortholog having at least 70% sequence identity thereto.

In some embodiments, the first U1A responsive RNA element is positioned in the 5′ UTR or the 3′ UTR of the nucleotide sequence encoding a first target polypeptide.

In some embodiments, when one or more additional combinations of a U1A responsive RNA element and a nucleotide sequence encoding a target polypeptide are present, each additional U1A responsive RNA element is positioned independently in the 5′

UTR or the 3′ UTR of the nucleotide sequence encoding the respective additional target polypeptide.

In various embodiments, the U1A responsive RNA element comprises the nucleotide sequence of SEQ ID No: 42, or a sequence having at least 70% sequence identity thereto.

In various embodiments, the polynucleotide comprises two, three, or four MSE sequences. In some embodiments, when two or more MSE sequences are present, at least two of these MSE sequences are targeted by the same miRNA. In some embodiments, when two or more MSE sequences are present, at least two of these MSE sequences are targeted by different miRNAs.

In various embodiments, the one or more MSE sequences are targeted by miRNA(s) which is characterized by tissue-specific, cell type-specific, cell-state-specific or species-specific expression. In some embodiments, the one or more MSE sequences is targeted by miR-1, miR-155, miR-124, miR-182, miR-96, miR-183, miR-21, miR-16, miR-17, miR-19, miR-25, miR-34, miR-92, miR-93, miR-142, miR-222, miR-149, miR-375, miR-503, or miR-181b-2.

In various embodiments, the first target polypeptide is a gene editing nuclease, a chimeric antigen receptor (CAR), an antibody, a cytotoxic protein, a cell surface receptor, a transcription factor, an enzyme, a reporter protein, a cytokine, or a polypeptide that confers drug resistance.

In various embodiments, the second target polypeptide is a gene editing nuclease, a chimeric antigen receptor (CAR), an antibody, a cytotoxic protein, a cell surface receptor, a transcription factor, an enzyme, a reporter protein, a cytokine, or a polypeptide that confers drug resistance.

In some embodiments, the gene editing nuclease is a Cas protein, ZFN nuclease, or TALEN. In one embodiment, the Cas protein is Cas9.

In some embodiments, the polynucleotide described herein is an RNA molecule.

In some embodiments, the polynucleotide described herein is a DNA molecule.

In another aspect, provided herein is a vector comprising the polynucleotide described herein. In some embodiments, the vector is a viral vector. In some embodiments, the viral vector is a retroviral vector, an adenovirus vector, a parvovirus vector, an influenza virus vector, a herpes virus vector, a poxvirus vector, a rhabdovirus vector, a paramyxovirus vector, a morbillivirus vector, or a reovirus vector. In some embodiments, the retroviral vector is a lentiviral vector. In some embodiments, the parvoviral vector is an adeno-associated virus (AAV) vector. In some embodiments, the paramyxovirus vector is a Sendai virus (SeV) vector.

In another aspect, provided herein is a composition comprising the polynucleotide or the vector described herein and a carrier or excipient. In some embodiments, the composition is a pharmaceutical composition comprising the polynucleotide or the vector and a pharmaceutically acceptable carrier or excipient.

In another aspect, provided herein is a host cell comprising the polynucleotide, or the vector described herein.

In another aspect, provided herein is a method of selectively expressing a payload polypeptide in a target cell type or tissue or a cell in a specific state within a multicellular organism, comprising introducing to the multicellular organism an effective amount of the polynucleotide, or the vector, or the composition described herein. In some embodiments, the multicellular organism is an animal, a plant, or a fungus. In some embodiments, the animal is a human, veterinary animal, or experimental animal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of the cardiovirus programmed ribosomal frameshifting (PRF) element. Figure discloses SEQ ID NO: 12.

FIG. 2 shows a schematic of the programmable ribosomal shift element of cardioviruses which, in the presence of the 2A protein, results in a ribosomal-1 shift.

FIGS. 3A-3B show the schematic of a bicistronic RNA encoding epitope tagged GFP and mCherry, the latter being in a −1 frame. Between the constructs is either a canonical stem loop (SL), or a mutated form lacking the cytosine triplet (SLmut), from EMCV which serves as the 2A-dependent ribosomal shift site (FIG. 3A). Western blot of protein derived from cells transfected with a wild type and mutated version of the constructs depicted in FIG. 3A with either 2A or a mutant form (m2A) (FIG. 3B).

FIGS. 4A-4B show the schematic of lentivirus constructs expressing a miR-21-targeted 2A transcript and an alternative splice site to generate mCherry (dashed lines). Downstream of the 3′ splice site contains the 2A-dependent ribosomal frame shift motif (FIG. 4A). In the presence of miR-21, 2A should be silenced and mCherry can be translated in original frame (frame 0). In the absence of miR-21, 2A creates a functional ribosomal frameshift element, resulting in change to the −1 frame for the cargo (mCherry) (FIG. 4A). If the cargo (mCherry) is constructed in frame 0, the presence of miR-21 and the absence of 2A will enable expression. If the cargo (mCherry) in constructed in the −1 frame, expression will be limited to conditions in which miR-21 is absent and 2A is present (FIG. 4A). Fluorescent microscopy of wild-type MDCK cells (MDCK^{WT}) and MDCK cells lacking miR-21 (MDCK^{miR-21-/-}) 2 days post transduction (2dpt) with a lentivirus depicted in FIG. 4A where the mCherry cargo was constructed to be in frame 0 relative to the ribosomal frame shift motif (FIG. 4B). Abbreviation used in the Figure: pA=Polyadenylation sequence.

FIGS. 5A-5B show various exemplary gene expression schemes utilizing programmable miRNA-dependent gene expression. The expression scheme can be utilized to express a single gene of interest (GOI) or multiplexed GOI through the use of alternative splicing events and/or multiple promoters. Abbreviations used in the Figure: P1=Promoter 1; pA=Polyadenylation sequence; I=Insulator; P2=Promoter 2; P3=Promoter 3.

FIG. 6 illustrates lentivirus- and AAV2-based miRNA-on switches with a single cytomegalovirus (CMV) promoter controlling cargo (mCherry) in frame 0 relative to the ribosomal frame shift motif in 293T cells. miR-21, but not miR-124, is present in 293T cells. Abbreviations used in the Figure: P1=Promoter 1; pA=Polyadenylation sequence.

FIG. 7 illustrates lentivirus-based miRNA-on switch with a single CMV promoter controlling cargo (mCherry) in frame 0 relative to the ribosomal frame shift motif in U-87 cells. miR-124, but not miR-21, is present in U-87 cells. Abbreviations used in the Figure: P1=Promoter 1; pA=Polyadenylation sequence; SL=stem loop.

FIG. 8 illustrates further exemplary gene expression schemes utilizing programmable miRNA-dependent gene expression. Abbreviations used in the Figure: P1=Promoter 1; pA=Polyadenylation sequence; P2=Promoter 2.

FIGS. 9A-9C illustrate a miRNA-dependent expression construct developed using a lentivirus delivery platform. Schematic of a plasmid encoding eGFP as two independent exons wherein the intron contains the pre-cursors for either Homo sapien miR-21 (hsa-miR-21) or miR-375 (hsa-miR-375) (FIG. 9A). Schematic of a lentiviral genome comprising a bi-directional promoter designed to express an EMCV 2A frameshift protein harboring two miR-21 target sites in the 3′ UTR and an in-frame mCherry transcript harboring a 5′ stem loop (SL) comprising the EMCV programable ribosomal shift (PRS) element (FIG. 9B). Fluorescent microscopy images of 293 cells co-transfected with the pLenti-2A-miR-21-PRS-mCherry plasmid and a pcDNA-eGFP construct expressing either miR-21 or miR-375 (FIG. 9C). Images were taken at 3 days post treatment. Top panels show mCherry expression and bottom panels show eGFP expression.

FIGS. 10A-10B illustrate a miRNA-dependent expression construct developed using a Sendai virus delivery platform. Schematic of an engineered Sendai virus genome encoding microRNA-21 (miR-21)-targeted (miR-21T) transcript encoding the EMCV 2A protein (2A) and an in-frame mCherry transcript harboring a 5′ stem loop (SL) comprising either a functional or mutated (SLmut) version of the EMCV programable ribosomal shift (PRS) element (FIG. 10A). Fluorescent microscopy images denoting mCherry expression in wild type Madin-Darby Canine Kidney (MDCK) cells, or a clonal MDCK line in which miR-21 expression was disrupted (MDCK^{miR-21-/-}) (FIG. 10B). Panels denote cells infected with the recombinant viruses depicted in FIG. 10A at a multiplicity of infection of 5 at 48 hours post infection.

FIGS. 11A-11C show results of an in vivo study using an engineered Sendai virus delivery platform. Schematics of a Sendai virus engineered to express the 2A protein with three miR-503 targets in its 3′ UTR (FIG. 11A). Red fluorescent protein (RFP) expression analyzed by immunohistochemistry (IHC) (FIG. 11B). A volcano plot of differentiated genes from RFP+ cells, showing that two genes, namely AQP5 and SFTPC, were significantly enriched (FIG. 11C).

FIGS. 12A-12C show IRP2-mediated translational control. FIG. 12A shows a schematic of Ferritin mRNA including the iron response element or iron-responsive element (IRE) hairpin in the 5′UTR. In the absence of IRP2, the ribosome (in grey) translates Ferritin without issue. FIG. 12B shows in the presence of IRP2, it binds the IRE and prevents ribosome-mediated translation. FIG. 12C shows in the presence of iron, or silencing or IRP2, Ferritin translation proceeds as normal.

FIG. 13 shows fluorescent microscopy of 293T cells co-transfected with p IRE-mClover or pIREmut-mClover and either pcDNA vector or pcDNA-IRP2.

FIG. 14A shows fluorescent microscopy of 293T cells co-transfected with pIRE-mClover and pIRP2-miR-122T and mimetics for either a scrambled miRNA mimetic (Negative), miR-122, or miR-375. FIG. 14B shows Western blot for IRP2 and beta actin from cells described in FIG. 14A.

FIGS. 15A-15C show a schematic representation of a miRNA-dependent genetic circuit. FIG. 15A shows an overview of the two-component system designed to regulate payload gene expression in a miRNA-dependent manner. The system consists of (1) an IRE-binding protein (e.g., IRP1 or IRP2) containing a 3′ UTR with two or more perfectly complementary target sites for the desired miRNA denoted as red circles, and (2) a payload gene with a 5′ UTR harboring an iron-responsive element (IRE). FIG. 15B shows that in the presence of the target miRNA, IRP2 is silenced via miRNA-mediated degradation or translational repression. As a result, the IRE in the payload's 5′ UTR remains unbound, allowing ribosomal translocation and subsequent payload expression. FIG. 15C shows that in the absence of the target miRNA, IRP2 remains expressed and actively binds to the IRE in the 5′ UTR of the payload gene. This binding blocks ribosomal translocation, preventing translation and effectively silencing the payload gene.

FIGS. 16A-16D show validation of the miRNA-dependent circuit using IRP2 and mClover reporters. FIG. 16A shows a schematic representation of pcDNA-IRP2, a eukaryotic expression vector encoding human IRP2 without 5′ or 3′ UTR regulatory elements. FIGS. 16B-16C show the design of mClover-expressing plasmids to assess IRP2-mediated translational repression. pIRE-mClover contains the wild-type 5′ UTR of the ferritin gene, including an intact iron-responsive element (IRE), while pIREmut-mClover harbors a mutated IRE resistant to IRP1- and IRP2-mediated repression. FIG. 16D shows fluorescence microscopy images of HEK293 cells transfected with equimolar amounts of either pIRE-mClover or pIREmut-mClover, in the presence of either an empty pcDNA vector (vector control) or pcDNA-IRP2 (pIRP2). Fluorescence was assessed 48 hours post-transfection (48 hpt) using an EVOS fluorescent microscope. Robust mClover fluorescence was observed in conditions lacking IRP2 or using pIREmut-mClover, while expression was strongly repressed in cells transfected with pIRE-mClover and pIRP2, confirming IRP2-mediated repression of IRE-containing transcripts.

FIGS. 17A-17C show validation of the miR-122-dependent IRP/IRE circuit in miRNA-deficient cells. FIG. 17A shows a schematic representation of pIRP2-miR-122T, a modified pIRP2 construct containing two perfect target sites for miR-122, a liver-specific miRNA. This design enables miR-122-dependent regulation of IRP2 expression. FIG. 17B shows the experimental workflow for testing miRNA-dependent control of the IRP/IRE circuit. Drosha-deficient HEK293 cells, which lack endogenous miRNAs but retain the necessary components for miRNA-mediated regulation, were transfected with synthetic miRNA mimetics for scrambled control (scrambled), miR-122, or miR-375 (a pancreatic islet-specific miRNA). Three days later, cells were co-transfected with pIRP2-miR-122T and pIRE-mClover, and fluorescence was assessed 48 hours post-transfection. Robust mClover expression was observed exclusively in miR-122-treated cells, whereas minimal to no fluorescence was detected in cells transfected with scrambled or miR-375 mimetics, confirming miR-122-dependent activation of the circuit. FIG. 17C shows Western blot analysis of IRP2 expression from whole-cell protein extracts. IRP2 protein was readily detected in conditions lacking miR-122, whereas its expression was significantly reduced in miR-122-transfected cells, further validating miRNA-dependent circuit regulation.

FIGS. 18A-18D show validation of the miR-122-dependent IRP/IRE circuit in a lentiviral-based delivery system. FIGS. 18A-18C show alternative vector configurations for circuit expression. The IRE/IRP-based circuit can be designed in multiple configurations: a single promoter-driven system utilizing alternative splicing to generate distinct transcripts for each element (FIG. 18A), a dual-promoter system with elements expressed under separate tandem promoters (FIG. 18B), or a bi-directional system (FIG. 18C), where each element is driven by opposing promoters to enable balanced expression. These configurations allow for flexible vector design, adaptable to different experimental and therapeutic applications, including lentivirus vectors, adenoviral vectors, adeno-associated viral (AAV) vectors, amongst others. Herein, configurations FIGS. 18A-18C are demonstrated in the context of a lentivirus vector. FIG. 18D shows the functional assessment of circuit expression following lentiviral transduction using the configuration shown in FIG. 18C. Drosha-deficient cells were pre-transfected with scrambled, miR-122, or miR-375 mimetics before transduction with Lenti-BiDi-IRP2-miR-122T-IRE-mClover. Fluorescence imaging 48 hours post-transduction showed that mClover expression occurred exclusively in cells transfected with miR-122, confirming that the circuit remains functional in a viral vector system.

FIGS. 19A-19B show miR-122-responsive lentivirus enabled by an IRP2-IRE-bi-directional switch. FIG. 19A shows a schematic of a lentiviral genome featuring a central bi-directional promoter. FIG. 19B shows fluorescent microscopy images of HEK293T cells treated with a scrambled miRNA mimetic (negative control #1), miR-122, or miR-375 and subsequently transduced with the lentivirus described in FIG. 19A 24 hrs thereafter. Images were obtained 48 hrs post transduction.

FIG. 20 shows fluorescent microscopy of 293T cells 48 hrs after being co-transfected with a plasmid encoding enhanced green fluorescent protein (eGFP) containing a 5′ IRE alongside a second plasmid encoding wild type Iron Response Protein 2 (WT IRP2), or IRP2 C-terminal truncations denoted by the number of amino acids remaining from the full-length 963-amino-acid protein (e.g. IRP2-900=IRP2 lacking the last 63 amino acids in the C-terminal end). Negative controls included non-transfected (NT) or IRE-eGFP (IRE) co-transfected with an empty vector (pcDNA).

FIG. 21 shows fluorescent microscopy of 293T cells 48 hrs after being co-transfected with a plasmid encoding enhanced green fluorescent protein (eGFP) containing a 5′ IRE alongside a second plasmid encoding wild type Iron Response Protein 2 (WT IRP2), or IRP2 mutants harboring internal deletions at the residues indicated (e.g. IRP2 Reg1Δ (2-318Δ)=IRP2 lacking amino acids 2-318; Reg=Region, indicating the domain that was deleted). Negative controls included non-transfected (NT) or IRE-eGFP (IRE) co-transfected with an empty vector (pcDNA).

FIG. 22 shows fluorescent microscopy of 293T cells 48 hrs after being co-transfected with a plasmid encoding enhanced green fluorescent protein (eGFP) containing a 5′ IRE alongside, a second plasmid encoding wild type Iron Response Protein 2 (WT IRP2), an empty vector (pcDNA), or one of three IRP2 mutants designed to disrupt FBXL5-mediated degradation of IRP2, which is the mechanism responsible for IRP2 degradation in high iron conditions (e.g. IRP2 iron sensitivity). These mutants are R779E-R763D mutations, C578S-C581S mutations, and a combined C578S-C581S-R779E-R763D mutant. The data suggest that R779 and R763 are essential for IRP2-mediated IRE repression, while C578 and C581 are not essential. IRP2-C578S-C581S can be a functional IRP2 variant that is not responsive to endogenous iron levels.

FIG. 23 shows fluorescent microscopy of Dicer-deficient 293T cells 48 hrs after being co-transfected with a plasmid encoding enhanced green fluorescent protein (eGFP) containing a 5′ IRE alongside a second plasmid comprising an empty vector (pcDNA), or encoding wild type Iron Response Protein 2 (WT IRP2) with three miRNA target sites for either miR-122 (IRP2 (miR-122T)) or miR-21 (IRP2 (miR-21T). 24 hrs prior to treatment, cells were transfected with a scrambled miRNA (Negative ctrl #1), or mimetics for miR-122 or miR-21. The data suggest that miRNA silencing of IRP2 is selective based on the miRNA target site used and miRNA available in the cell, and the combined system can be used to turn on gene expression in the presence of particular miRNA.

FIG. 24 shows a schematic of an exemplary miRNA-dependent circuit design in a Sendai virus vector using a degron (e.g., hPEST) to modulate levels of IRP2.

FIGS. 25A-25C show a schematic representation of another miRNA-dependent genetic circuit. FIG. 25A shows an overview of a two-component system designed to regulate payload gene expression in a miRNA-dependent manner. The system consists of (1) a gene encoding an RNA-binding protein, Lin28, containing a 3′ UTR with two or more perfectly complementary target sites for the desired miRNA denoted as red circles, and (2) a payload gene with a 5′ UTR harboring an RNA element derived from a let-7 miRNA precursor (pre-let-7). FIG. 25B shows that in the presence of the target miRNA, Lin28 is silenced via miRNA-mediated degradation or translational repression. As a result, the pre-let-7 element in the payload's 5′ UTR remains unbound, allowing ribosomal translocation and subsequent payload expression. FIG. 25C shows that in the absence of the target miRNA, Lin28 remains expressed and actively binds to the pre-let-7 element in the 5′ UTR of the payload gene. This binding blocks ribosomal translocation, preventing translation and effectively silencing the payload gene.

FIG. 26A shows genome schematics of six Sendai vectors illustrating placement of the LET7D mScarlet cassette and the Lin28A cassette bearing four miR-122 target sites. The construct NP_plet7d-mScarlet encodes LET7D mScarlet between N and P. The construct PM_plet7d-mScarlet carries the same cassette between P and M. The construct NP_LIN-122T (4×) encodes Lin28A with four tandem miR-122 sites at the N to P junction, and PM_LIN-122T (4×) encodes the same cassette between P and M. Two combined vectors were also generated. In NP_plet7d-mScarlet PM_LIN-122T (4×), LET7D mScarlet is placed between N and P while Lin28A-122T is placed between P and M. In the reciprocal arrangement, NP_LIN-122T (4×) PM_plet7d-mScarlet, the Lin28A-122T cassette is positioned between N and P and the LET7D mScarlet cassette is positioned between P and M. All other Sendai genes are shown in gray. FIG. 26B shows fluorescence outputs from the individual vectors following infection of A549 cells lacking miR-122 and Huh7.5 cells expressing miR-122 at a multiplicity of infection of three. The LET7D mScarlet only vectors NP_plet7d-mScarlet and PM_plet7d-mScarlet produced red fluorescence in both cell types. The Lin28A only vectors NP_LIN-122T (4×) and PM_LIN-122T (4×) produced no signal, consistent with the absence of an mScarlet gene. The combined construct NP_plet7d-mScarlet PM_LIN-122T (4×) produced mScarlet fluorescence selectively in Huh7.5 cells, indicating miR-122 dependent activation of the LET7D module. The reciprocal construct NP_LIN-122T (4×) PM_plet7d-mScarlet showed no detectable fluorescence in either cell type, demonstrating that gene order within the Sendai genome strongly influences output from the coupled system.

FIGS. 27A-27C show a schematic representation of another miRNA-dependent genetic circuit. FIG. 27A shows an overview of a two-component system designed to regulate payload gene expression in a miRNA-dependent manner. The system consists of (1) a gene encoding an RNA-binding protein, MS2 bacteriophage coat protein (MS2), containing a 3′ UTR with two or more perfectly complementary target sites for the desired miRNA denoted as red circles, and (2) a payload gene with a 5′ UTR harboring an MS2 responsive RNA element. FIG. 27B shows that in the presence of the target miRNA, MS2 is silenced via miRNA-mediated degradation or translational repression. As a result, the MS2 responsive RNA element in the payload's 5′ UTR remains unbound, allowing ribosomal translocation and subsequent payload expression. FIG. 27C shows that in the absence of the target miRNA, MS2 remains expressed and actively binds to the MS2 responsive RNA element in the 5′ UTR of the payload gene. This binding blocks ribosomal translocation, preventing translation and effectively silencing the payload gene.

FIG. 28 shows fluorescent microscopy of miRNA positive BsrT7 cells and miRNA deficient NoDice 293T cells transfected with a GFP reporter containing a 5′ MS2 responsive RNA element (scMS2 GFP). Cells were transfected with scMS2 GFP alone, scMS2 GFP plus an MS2 expression plasmid, or scMS2 GFP plus an MS2 plasmid containing target sites for miR-21, miR-18a, and miR-20 in its 3′ untranslated region. Reporter expression is suppressed when MS2 is present and is restored only when MS2 is selectively silenced by the endogenous miRNAs. Images were acquired 48 hours after transfection.

FIGS. 29A-29C show a schematic representation of another miRNA-dependent genetic circuit. FIG. 29A shows an overview of a two-component system designed to regulate payload gene expression in a miRNA-dependent manner. The system consists of (1) a gene encoding an RNA-binding protein, PP7 bacteriophage coat protein (PP7), containing a 3′ UTR with two or more perfectly complementary target sites for the desired miRNA denoted as red circles, and (2) a payload gene with a 5′ UTR harboring an PP7 responsive RNA element. FIG. 29B shows that in the presence of the target miRNA, PP7 is silenced via miRNA-mediated degradation or translational repression. As a result, the PP7 responsive RNA element in the payload's 5′ UTR remains unbound, allowing ribosomal translocation and subsequent payload expression. FIG. 29C shows that in the absence of the target miRNA, PP7 remains expressed and actively binds to the PP7 responsive RNA element in the 5′ UTR of the payload gene. This binding blocks ribosomal translocation, preventing translation and effectively silencing the payload gene.

FIG. 30 shows fluorescent microscopy of miRNA positive BsrT7 cells and miRNA deficient NoDice 293T cells transfected with a GFP reporter containing a 5′ PP7 responsive RNA element (scPP7 GFP). Cells were transfected with scPP7 GFP alone, scPP7 GFP plus a PP7 expression plasmid, or scPP7 GFP plus a PP7 plasmid containing target sites for miR-21, miR-18a, and miR-20 in its 3′ untranslated region. Reporter expression is suppressed when PP7 is present and is restored only when PP7 is selectively silenced by the endogenous miRNAs. Images were acquired 48 hours after transfection.

FIGS. 31A-31C show a schematic representation of another miRNA-dependent genetic circuit. FIG. 31A shows an overview of a two-component system designed to regulate payload gene expression in a miRNA-dependent manner. The system consists of (1) a gene encoding an RNA-binding protein, U1 small nuclear ribonucleoprotein A (U1A), containing a 3′ UTR with two or more perfectly complementary target sites for the desired miRNA denoted as red circles, and (2) a payload gene with a 5′ UTR harboring an U1A responsive RNA element. FIG. 31B shows that in the presence of the target miRNA, U1A is silenced via miRNA-mediated degradation or translational repression. As a result, the U1A responsive RNA element in the payload's 5′ UTR remains unbound, allowing ribosomal translocation and subsequent payload expression. FIG. 31C shows that in the absence of the target miRNA, U1A remains expressed and actively binds to the U1A responsive RNA element in the 5′ UTR of the payload gene. This binding blocks ribosomal translocation, preventing translation and effectively silencing the payload gene.

FIG. 32 shows fluorescent microscopy of miRNA positive BsrT7 cells and miRNA deficient NoDice 293T cells transfected with a GFP reporter containing a 5′ U1A responsive RNA element (U1utr GFP). Cells were transfected with U1utr GFP alone, U1utr GFP plus a U1 expression plasmid, or U1utr GFP plus a U1 plasmid containing target sites for miR-21, miR-18a, and miR-20 in its 3′ untranslated region. Reporter expression is suppressed when U1A is present and is restored only when U1A is selectively silenced by the endogenous miRNAs. Images were acquired 48 hours after transfection.

### DETAILED DESCRIPTION

One strategy to restrict expression of RNA- or DNA-based therapies to minimize off-target effects is the exploitation of miRNAs5-9. In most eukaryotic organisms, small noncoding RNAs called microRNAs (miRNAs) fine-tune protein expression by engaging mRNAs to induce de-adenylation and/or stall the ribosome during the translation process¹⁰. This biology orchestrates various developmental processes and is essential for maintaining most cell lineages in the body. In contrast to antiviral RNA interference (RNAi) utilized by plants and arthropods where the small RNA is captured from incoming virus, miRNAs derive from host-encoded RNA hairpins and do not participate in the host defenses¹¹. A miRNA is loaded into an RNA-induced silencing complex (RISC) and engages its target mRNAs in a sequence-specific manner. In contrast to the small interfering RNAs (siRNA) generated during the antiviral response of plants and arthropods, the impact a miRNA has on its cognate host target(s) is determined by the extent of complementarity. Typically, binding of a miRNA to the target mRNA is not contiguous like it is for antiviral RNAi, leading to only moderate changes in target RNA regulation and protein output with levels ranging from 1.1- to 1.5-fold reductions¹¹. The modest repression induced by miRNAs do not elicit drastic changes in the cell when measured over days. However, miRNA biology can dramatically influence cellular processes over the course of weeks to months and are ideal modulators of cell development and differentiation. For this reason, most cell lineages harbor a distinct profile of miRNAs although examples of ubiquitous species are also common. As this modest level of repression is a product of target complementarity, should a miRNA engage an mRNA containing a target with perfect complementarity, the mRNA would be enzymatically cleaved, leading to complete ablation of the message akin to siRNA biology¹². While this biology does not naturally occur in vertebrates, it can be engineered and applied to both mRNA and vector-based designs to control tropism. Indeed, there are now many reports of successful designs where such strategies have successfully limited the capacity of a given genetic circuit to be silenced in a specific cell lineage or tissue based on the unique expression of a miRNA. Despite the value in excluding expression of a biologic target in unwanted tissues, application of this concept does not enable one to achieve cell-specific expression unless every unwanted cell type both expressed a unique miRNA and a target for each tissue could be incorporated into the vector of interest. This is not possible because the miRNA expression profiles of all cell types are not known and, if this knowledge were obtained, the space constraints to include each target would be prohibitive as cargo capacity in most clinical vectors is finite. As a result, a new strategy for achieving cell-specificity is needed.

The miRNA-dependent polynucleotide circuits described in the present disclosure address the critical challenge of achieving cell type-specific delivery of therapeutic cargos, a major barrier in developing gene and cell therapies. By leveraging the fact that nearly every tissue expresses at least one highly conserved miRNA across vertebrates, the miRNA-dependent circuits described herein offer an unprecedented level of precision in gene regulation. Current strategies often struggle with off-target effects, inefficient delivery, lack of tissue or cell specificity, limited cargo capacity, and the need for strict regulatory control to prevent unintended consequences. The described RNA-based circuit can leverage a host protein, such as IRP2, which binds to the iron response element (IRE) in mRNA, to enable miRNA-dependent translation of target genes. It uniquely demands the presence of a specific miRNA for gene expression, offering a more precise and programmable approach compared to existing technologies as the vast majority of cell types express at least one unique miRNA. This level of specificity could dramatically improve the safety and efficacy of gene therapy applications, reducing concerns related to ectopic expression and associated toxicities. Beyond improved specificity, other advantages include the use of host-derived components to prevent cytotoxicity and/or immunogenicity and versatility as the described circuit should work across any vector-based platform. The miRNA-gated circuitry represents a customizable genetic delivery system with a transformative advancement in gene therapy, improving both the safety and efficacy of viral vector-based interventions.

Incorporation of the miRNA-dependent circuits described herein can ensure that a given mRNA- or vector-based therapeutics delivers its biological payload exclusively to a given cell type of interest. The capacity to achieve this minimizes toxicity and immunogenicity while improving therapeutic outcomes.

### Definitions

The term “microRNA (miRNA) Silencing Element” or “MSE” is used herein to refer to a nucleotide sequence within an mRNA that can bind to a specific miRNA and result in a measurable amount of post-transcriptional silencing of such mRNA (determined, e.g., by a decrease in mRNA and/or protein content). For post-transcriptional silencing to occur, MSE-miRNA sequence complementarity in most cases will include the seed sequence of the miRNA, which is typically comprised of nucleotides 1-7 or 2-8 of the miRNA, and additional complementarity following the seed sequence.

The term “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

In reference to the nucleic acid molecules of the present disclosure, the binding free energy for a nucleic acid molecule with its complementary sequence is sufficient to allow the relevant function of the nucleic acid to proceed, e.g., miRNA activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J Am. Chem. Soc. 109:3783-3785). “Perfectly complementary” means that all the contiguous residues of a nucleic acid sequence will form hydrogen bond(s) with the same number of contiguous residues in a second nucleic acid sequence. In one embodiment, the human miRNA has partial complementarity (i.e., less than 100% complementarity) with the corresponding target nucleic acid molecule.

The term “ribosomal frameshift stimulator”, as used herein, refers to a trans-acting proteinous molecule that can promote a ribosomal frameshift in an mRNA transcript.

The term “ribosomal frameshift element” or “RFE”, as used herein, refers to a cis-acting polynucleotide sequence that can cause a ribosomal frameshift in an mRNA transcript in the presence of a ribosomal frameshift stimulator.

As used herein, the term “multicellular organism” refers to an individual organism consisting of a plurality of cells (typically, a plurality of cells of different types). Multicellular organisms include animals, plants, fungi, and the like. In some embodiments, animals refer to mammals including humans, veterinary animals (e.g., cats, dogs, cows, horses, sheep, pigs, etc.) and experimental animal models (e.g., mice, rats, monkeys, etc.). In one embodiment, the animal is a human.

As used herein, a “functional variant” is a polypeptide or protein that differs from a reference sequence (e.g., by one or more substitutions, insertions, deletions, truncations, extensions, fusions, or post-translational/chemical modifications) but retains a specified biological activity of the reference in a relevant assay. The retained activity may include, for example, binding to the same target (e.g., an RNA element such as iron response element), catalyzing the same reaction, signaling through the same pathway, eliciting the same epitope/immune response, or producing a substantially similar phenotypic effect. In some embodiments, a functional variant binds the same target (e.g., an RNA element such as iron response element) within 2-10 times affinity (Kd); exhibits at least 50%, 70%, 80%, 90%, or 100-150% of the reference activity; or yields comparable functional outcomes. Functional variants can include conservative-substitution variants, allelic variants, isoforms, orthologs, homologs, functional fragments containing essential domains, and fusion proteins that retain the relevant function (e.g., binding to an RNA element such as iron response element).

It will be appreciated that conservative amino acid substitutions may be introduced to the polypeptide of any of those described herein, to attain a polypeptide having, for example, 60%, 65%, 70%, 75%, 80%, 85%, 90%, 95%, 98%, or 99% sequence identity to the referenced sequence, and maintaining similar or the same activity of that sequence. Conservative amino acid substitutions involve substituting amino acids in a protein with amino acids having similar side chains in regard to, for example, chemical properties, structure, and/or size. As an example, without limitation, the amino acids within each of the following groups may be interchanged with other amino acids in the same group: amino acids having acidic side chains, such as aspartic acid and glutamic acid; amino acids having non-aromatic, hydroxyl-containing side chains, such as serine and threonine; amino acids having aliphatic side chains, including glycine, alanine, valine, leucine and isoleucine; amino acids having sulfur-containing side chains, including cysteine and methionine; amino acids having amide side chains, including glutamine and asparagine; basic amino acids, including lysine, arginine, and histidine; and amino acids having aromatic ring side chains, including phenylalanine, tyrosine and tryptophan. Furthermore, amino acids having acidic side chains, such as glutamic acid and aspartic acid, are regarded as interchangeable herein with amino acids having amide side chains, such as glutamine and asparagine. Peptide insertions may comprise one or more (for example, 1, 2, 3, 4, 5, 6, 7, 8, 9, or 10) amino acid substitutions, and/or insertions, and/or deletions. Amino acid substitution means that an amino acid residue is substituted for a replacement amino acid residue at the same position. Inserted amino acid residues may be inserted at any position and may be inserted such that some or all of the inserted amino acid residues are immediately adjacent to one another or may be inserted such that none of the inserted amino acid residues is immediately adjacent to another inserted amino acid residue. One or more (for example, 1, 2, 3, 4, 5, 6, 7, 8, 9, or 10) amino acids may be substituted, and/or inserted, and/or deleted from the sequence of the reference polypeptide. Each substitution, and/or insertion, and/or deletion can take place at any position of the amino acid sequence.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

Percent sequence identity can be determined using a global alignment between two sequences. As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see ebi.ac.uk/Tools/psa/emboss_needle/) or the global alignment tool “BLAST®»Global Alignment” from the National Center for Biotechnology Information (NCBI) (see blast.ncbi.nlm.nih.gov/Blast.cgi?PROGRAM=blastn&PAGE_TYPE=BlastSearch&PROG_D EFAULTS=on&BLAST_INIT=GlobalAln&BLAST_SPEC=GlobalAln&BLAST_PROGRA MS=blastn). Both of these global alignment tools incorporate the Needleman-Wunsch algorithm (Needleman, S.B. & Wunsch, C.D. (1970) “A general method applicable to the search for similarities in the amino acid sequences of two proteins.” J. Mol. Biol. 48:443-453). In some embodiments, a global alignment of nucleotide sequences using BLAST Global Alignment uses the following default parameters: match score=2; mismatch score=−3; Gap Cost Existence score=5; Gap Cost Extension Score=2. In some embodiments, a global alignment of protein sequences using BLAST Global Alignment uses the following default parameters: Gap Cost Existence=11; Gap Cost Extension=1.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked polypeptide is one in which the functional domains are placed with appropriate distance from each other to impart the intended function of each domain.

As used herein “specifically bind” or “specific binding” refers to an interaction between an RNA-binding protein and an RNA element in which the RNA-binding protein recognizes and associates with the RNA element in a manner that is measurably distinguishable from non-specific interactions. Specific binding is characterized by a higher affinity for the intended target RNA element relative to unrelated RNA targets or nucleic acids under comparable conditions. Specific binding may be determined using any conventional biochemical or biophysical assay, including but not limited to electrophoretic mobility shift assays (EMSA), RNA immunoprecipitation (RIP), surface plasmon resonance (SPR), isothermal titration calorimetry (ITC), or crosslinking assays. An RNA-binding protein is considered to specifically bind an RNA element when the dissociation constant (K_{D}), association rate, or other measurable parameter indicates selective recognition.

The term “therapeutically effective amount” refers to that quantity of a compound (e.g., polynucleotide, vector) or a pharmaceutical composition comprising such compound that is sufficient to result in a desired activity upon administration to a subject in need thereof. Within the context of the present invention, the term “therapeutically effective” refers to that quantity of a compound or pharmaceutical composition that is sufficient to achieve selective expression of a target polypeptide in a target cell type or tissue or a cell in a specific state.

The phrase “pharmaceutically acceptable” refers to molecular entities and compositions that are physiologically tolerable and do not typically produce an allergic or similar untoward reaction, when administered to a mammal (e.g., human). Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” applied to pharmaceutical compositions of the invention refers to a diluent, excipient, or vehicle with which a compound (e.g., a polynucleotide or vector) is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82:488-492 (1985), U.S. Pat. No. 5,071,743, Fukuoka et al., Biochem. Biophys. Res. Commun. 263:357-360 (1999); Kim and Maas, BioTech. 28:196-198 (2000); Parikh and Guengerich, BioTech. 24:4 28-431 (1998); Ray and Nickoloff, BioTech. 13:342-346 (1992); Wang et al., BioTech. 19:556-559 (1995); Wang and Malcolm, BioTech. 26:680-682 (1999); Xu and Gong, BioTech. 26:639-641 (1999), U.S. Pat. Nos. 5,789,166 and 5,932,419, Hogrefe, Strategies 14. 3:74-75 (2001), U.S. Pat. Nos. 5,702,931, 5,780,270, and 6,242,222, Angag and Schutz, Biotech. 30:486-488 (2001), Wang and Wilkinson, Biotech. 29:976-978 (2000), Kang et al., Biotech. 20:44-46 (1996), Ogel and McPherson, Protein Engineer. 5:467-468 (1992), Kirsch and Joly, Nuc. Acids. Res. 26:1848-1850 (1998), Rhem and Hancock, J. Bacteriol. 178:3346-3349 (1996), Boles and Miogsa, Curr. Genet. 28:197-198 (1995), Barrenttino et al., Nuc. Acids. Res. 22:541-542 (1993), Tessier and Thomas, Meths. Molec. Biol. 57:229-237, and Pons et al., Meth. Molec. Biol. 67:209-218.

The technology illustratively described herein suitably may be practiced in the absence of any element(s) not specifically disclosed herein. The terms and expressions which have been employed are used as terms of description and not of limitation, and use of such terms and expressions do not exclude any equivalents of the features shown and described or portions thereof, and various modifications are possible within the scope of the technology claimed.

microRNA (miRNA)-Dependent Circuits

In one aspect, described herein are miRNA-dependent polynucleotide circuits which can be programmed to express a polypeptide payload in a target cell type or tissue, a cell in a specific state, or a target species.

In various embodiments, the present disclosure provides a polynucleotide comprising: a) a nucleotide sequence encoding an RNA-binding protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the RNA-binding protein to allow expression of the RNA-binding protein in the absence of the respective miRNA targeting said MSE sequence; c) a first RNA element to which the RNA-binding protein specifically binds; d) a nucleotide sequence encoding a first target polypeptide, wherein the first RNA element is positioned with respect to the nucleotide sequence encoding the first target polypeptide to allow translation of the first target polypeptide in either the presence or absence of the RNA-binding protein; and e) optionally one or more additional combinations of an RNA element to which the RNA-binding protein specifically binds and a nucleotide sequence encoding a target polypeptide, wherein within each combination, the RNA element is positioned with respect to the nucleotide sequence encoding the target polypeptide to allow translation of the target polypeptide in either the presence or absence of the RNA-binding protein, wherein translation of each additional target polypeptide is controlled by its closest RNA element in the resulting mRNA.

In various embodiments, the present disclosure provides a system comprising: (i) a first polynucleotide comprising a) a nucleotide sequence encoding an RNA-binding protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the RNA-binding protein to allow expression of the RNA-binding protein in the absence of the respective miRNA targeting said MSE sequence; and (ii) a second polynucleotide comprising c) a first RNA element to which the RNA-binding protein specifically binds; d) a nucleotide sequence encoding a first target polypeptide, wherein the first RNA element is positioned with respect to the nucleotide sequence encoding the first target polypeptide to allow translation of the first target polypeptide in either the presence or absence of the RNA-binding protein. Optionally, the system further comprises (as part of the first polynucleotide, part of the second polynucleotide or as one or more additional polynucleotides) one or more additional combinations of an RNA element to which the RNA-binding protein specifically binds and a nucleotide sequence encoding a target polypeptide, wherein within each combination, the RNA element is positioned with respect to the nucleotide sequence encoding the target polypeptide to allow translation of the target polypeptide in either the presence or absence of the RNA-binding protein, wherein translation of each additional target polypeptide is controlled by its closest RNA element in the resulting mRNA.

In some embodiments, the RNA element to which the RNA binding protein specifically binds comprise at least about 25 nucleotides, at least about 30 nucleotides, at least about 35 nucleotides, at least about 40 nucleotides, at least about 45 nucleotides, at least about 50 nucleotides, at least about 55 nucleotides, or at least about 60 nucleotides.

In some embodiments, the RNA element to which the RNA-binding protein specifically binds comprise about 25 to 35 nucleotides, about 25 to 40 nucleotides, about 25 to 50 nucleotides, about 25 to 60 nucleotides, about 25 to 70 nucleotides, about 25 to 80 nucleotides, about 30 to 40 nucleotides, about 30 to 50 nucleotides, about 30 to 60 nucleotides, about 30 to 70 nucleotides, about 30 to 80 nucleotides, about 40 to 60 nucleotides, about 40 to 70 nucleotides, about 40 to 80 nucleotides.

In some embodiments, the polynucleotide circuit described herein is an RNA molecule.

In some embodiments, the polynucleotide circuit described herein is a DNA molecule.

Ribosomal Frameshift Element (RFE)-Based miRNA-Dependent Circuits

In some embodiments, the present disclosure provides a polynucleotide comprising: a) a nucleotide sequence encoding a ribosomal frameshift stimulator; b) one or more microRNA (miRNA) Silencing Element (MSE) sequences, wherein each of the one or more MSE sequences is targeted by a miRNA, and wherein each of the one or more MSE sequences is positioned with respect to the nucleotide sequence encoding the ribosomal frameshift stimulator to allow expression of the ribosomal frameshift stimulator in the absence of the respective miRNA targeting the MSE sequence; c) a first ribosomal frameshift element; and d) a nucleotide sequence encoding a first target polypeptide, wherein the first ribosomal frameshift element is positioned 5′ to the nucleotide sequence encoding the first target polypeptide, and wherein translation of the first target polypeptide is controlled by the first ribosomal frameshift element and requires either the presence or absence of the ribosomal frameshift stimulator. Optionally, the polynucleotide may further comprise one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

In one embodiment, the present disclosure provides a system comprising: (i) a first polynucleotide comprising a) a nucleotide sequence encoding a ribosomal frameshift stimulator; b) one or more microRNA (miRNA) Silencing Element (MSE) sequences, wherein each of the one or more MSE sequences is targeted by a miRNA, and wherein each of the one or more MSE sequences is positioned with respect to the nucleotide sequence encoding the ribosomal frameshift stimulator to allow expression of the ribosomal frameshift stimulator in the absence of the respective miRNA targeting the MSE sequence; and (ii) a second polynucleotide comprising c) a first ribosomal frameshift element; and d) a nucleotide sequence encoding a first target polypeptide, wherein the first ribosomal frameshift element is positioned 5′ to the nucleotide sequence encoding the first target polypeptide, and wherein translation of the first target polypeptide is controlled by the first ribosomal frameshift element and requires either the presence or absence of the ribosomal frameshift stimulator. Optionally, the system further comprises (as part of the first polynucleotide, part of the second polynucleotide or as one or more additional polynucleotides) one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

In various embodiments, a polynucleotide molecule described herein comprises a nucleotide sequence encoding a ribosomal frameshift stimulator. The one or more MSE sequences are positioned with respect to the nucleotide sequence encoding the ribosomal frameshift stimulator to allow expression of the ribosomal frameshift stimulator in the absence of the respective miRNA targeting the MSE sequence.

In some embodiments, the one or more MSE sequences are positioned in the open reading frame (ORF) or in 3′ untranslated region (3′ UTR) of the ribosomal frameshift stimulator transcript coding sequence.

In some embodiments, the ribosomal frameshift stimulator is a cardiovirus 2A protein, or a functional variant or ortholog thereof.

Cardioviruses are a genus of viruses that belong to the Picornaviridae family. There are currently six species in the genus Cardiovirus A, Cardiovirus B, Cardiovirus C, Cardiovirus D, Cardiovirus E, and Cardiovirus F. Cardiovirus A is composed of one serotype, encephalomyocarditis virus (EMCV). Cardiovirus B comprises four known viruses: Theiler's Murine encephalomyelitis virus (TMEV), Vilyuisk human encephalomyelitis virus (VHEV), a Theiler-like rat virus (TRV) and Saffold virus (SAF-V).

In some embodiments, the cardiovirus 2A protein is derived from EMCV, or a variant thereof. In other embodiments, the cardiovirus 2A protein may be derived from TMEV, VHEV, TRV or SAF-V, or a variant thereof.

Despite the low sequence identity amongst Cardiovirus 2A orthologs, they adopt a common architecture. They form a so-called β₃αβ₃αβ ‘β-shell’ fold comprised of six- or seven-stranded antiparallel β-sheet, packed against two a-helices. One of the most conserved elements is the flexible “arginine loop”, located between β5 and β6. This loop can be essential for both RNA binding and frameshifting. See, Napthine, S. et al. Protein-directed ribosomal frameshifting temporally regulates gene expression. Nat Commun 8, 15582 (2017), Finch, L. et al., Characterization of Ribosomal Frameshifting in Theiler's Murine Encephalomyelitis Virus. J Virol. 2015 August; 89 (16): 8580-9, Napthine, S. et al., Characterization of the stimulators of protein-directed ribosomal frameshifting in Theiler's murine encephalomyelitis virus. Nucleic Acids Res. 2019 Sep. 5; 47 (15): 8207-8223; each of which is incorporated by reference in its entirety.

In some embodiments, the cardiovirus 2A protein comprises the amino acid sequence

MSPNALDISRTYPTLHVLIQFNHRGLEVRLFRHGHFWAETRADVILRSKTKQVSFLSN GNYPSMDSRAPWNPWKNTYQAVLRAEPCRVTMDIYYKRVRPFRLPLVQKEWPVRE ENVFGLYRIFNAHYAGYFADLLIHDIETNPG (SEQ ID NO: 1), or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 1. In one embodiment, the cardiovirus 2A protein comprises the amino acid sequence of SEQ ID NO: 1. In one embodiment, the cardiovirus 2A protein consists essentially of the amino acid sequence of SEQ ID NO: 1. In one embodiment, the cardiovirus 2A protein consists of the amino acid sequence of SEQ ID NO: 1.

In some embodiments, the nucleotide sequence encoding the cardiovirus 2A protein comprises the nucleotide sequence

ATGAGTCCAAATGCCCTAGACATTTCAAGAACATACCCCACGTTACATGTTCTCA TTCAATTCAACCATAGAGGTTTGGAGGTTAGATTGTTTAGACATGGACACTTTTG GGCTGAAACACGTGCGGACGTGATTCTGAGATCAAAGACCAAACAGGTCTCTTT CCTGAGCAACGGGAACTACCCGTCAATGGACTCTAGAGCTCCCTGGAATCCTTGG AAGAATACCTACCAGGCGGTTCTAAGAGCAGAACCATGTAGAGTGACCATGGAT ATATATTATAAGAGAGTCAGGCCTTTTAGACTGCCCCTGGTTCAGAAGGAATGGC CCGTGCGAGAGGAGAACGTTTTCGGTTTGTACCGGATCTTCAATGCCCACTACGC TGGTTACTTTGCGGACCTACTGATTCATGACATTGAGACAAATCCAGGGTAG (SEQ ID NO: 2), or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 2. In one embodiment, the nucleotide sequence encoding the cardiovirus 2A protein comprises the nucleotide sequence of SEQ ID NO: 2. In one embodiment, the nucleotide sequence encoding the cardiovirus 2A protein consists essentially of the nucleotide sequence of SEQ ID NO: 2. In one embodiment, the nucleotide sequence encoding the cardiovirus 2A protein consists of the nucleotide sequence of SEQ ID NO: 2.

In some embodiments, the nucleotide sequence encoding the cardiovirus 2A protein is codon optimized for expression in a target species.

In some embodiments, a ribosomal frameshift element used in a miRNA-dependent polynucleotide circuit described herein is derived from a cardiovirus.

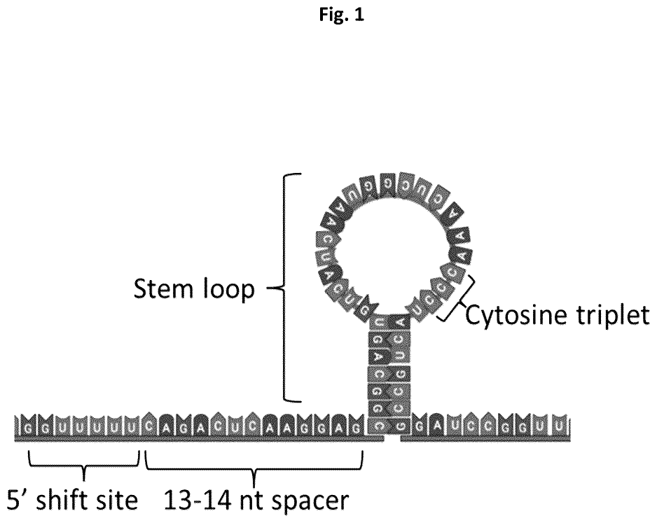

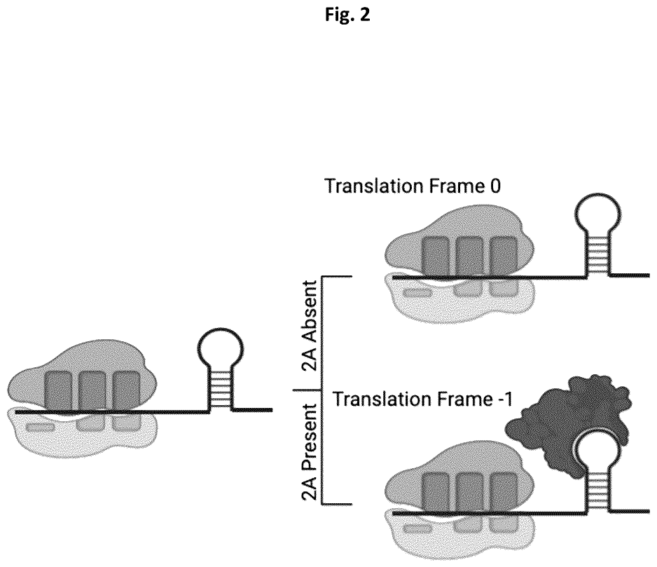

FIGS. 1-2 illustrate the ribosomal frameshift biology utilized by the family of cardioviruses. In the context of cardioviruses, a stretch of uracil bases upstream of an RNA hairpin induce a consistent ribosomal slippage event during translation¹³. Similar to many frameshift motifs utilized by viruses, a stretch of ~5 uracil bases located 12-15nts upstream of a structure that induces a steric hindrance on the ribosome such as a stable GC-rich RNA hairpin. During translation, the ribosome is stalled in place as it advances into the hairpin as the active peptidyl transferase site is positioned directly on the stretch of uracils, causing slippage and a change in reading frame. While ribosomal frame shift elements can function based entirely on the RNA motif described, cardioviruses impose an additional level of control onto the element by evolving the need for a protein (2A protein) to stabilize the hairpin and impose the steric hindrance on the ribosome-enabling utilization of a −1 reading frames¹³. Ribosomal frame shift phenomena are further described in Hill, C. H. et al. Structural and molecular basis for cardiovirus 2A protein as a viral gene expression switch. Nat Commun 12, 7166 (2021), Napthine, S. et al. Protein-directed ribosomal frameshifting temporally regulates gene expression. Nat Commun 8, 15582 (2017), and Loughran, G. et al. Ribosomal frameshifting into an overlapping gene in the 2B-encoding region of the cardiovirus genome. PNAS 108, 46, 1111 (2011), each of which is incorporated by reference in its entirety.

Cardiovirus programmed ribosomal frameshifting (PRF) system typically requires:
1. 5′ shift site composed of GGUUUUU;
2. 3′ adjacent to the shift site is a stimulatory RNA spacer of 13-14 nts;
3. 3′ adjacent to the stimulatory RNA spacer is a stem loop comprising a cytosine triplet in the loop; and
4. a cardiovirus 2A protein capable of recognizing the RNA stem loop.

In some embodiments, the ribosomal frameshift element is derived from EMCV, or a variant thereof. In some embodiments, the ribosomal frameshift element is derived from TMEV, VHEV, TRV or SAF-V, or a variant thereof.

In some embodiments, a ribosomal frameshift element used in a miRNA-dependent polynucleotide circuit described herein comprises from 5′ to 3′: 1) a 5′ shift site, 2) a spacer sequence, and 3) a stem-loop structure.

In some embodiments, the 5′ shift site comprises the sequence 5′-GGUUUUU-3′. In some embodiments, the 5′ shift site consists essentially of the sequence 5′-GGUUUUU-3′. In some embodiments, the 5′ shift site consists of the sequence 5′-GGUUUUU-3′.

In some embodiments, the spacer is 13-17 nucleotides long. In some embodiments, the spacer is 13, 14, 15, 16 or 17 nucleotides long. The spacer may not need to be sequence specific.

In some embodiments, the spacer is 13 or 14 nucleotides long. In some embodiments, the spacer comprises the sequence CAGACUCAAGGAG (SEQ ID NO: 4), or a sequence having at least 35%, at least 45%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 4. In some embodiments, the spacer comprises the sequence SEQ ID NO: 4. In some embodiments, the spacer consists essentially of the sequence SEQ ID NO: 4. In some embodiments, the spacer consists of the sequence SEQ ID NO: 4.

In some embodiments, the stem-loop structure comprises a stem of about 7-10 nucleotides. In some embodiments, the stem-loop structure comprises a stem of about 7, 8, 9 or 10 nucleotides. In some embodiments, the stem-loop structure comprises a stem of about 7 nucleotides. In some embodiments, the stem-loop structure comprises the sequence 5′-CGGCAGU-3′ followed by a loop and the complementary sequence 5′ACUGCCG-3′.

In some embodiments, the loop comprises about 18-30 nucleotides. In some embodiments, the loop comprises 18, 19, 20, 21, 22, 23, 24, 25, 26, 27, 28, 29, or 30 nucleotides. In some embodiments, the loop comprises about 21 nucleotides. In some embodiments, the loop of the stem-loop structure comprises an adenosine triplet followed by a cytosine triplet. In some embodiments, the loop structure comprises the sequence GUCAUCAAUGGCUCAAACCCU (SEQ ID NO: 7), or a sequence having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 7. In some embodiments, the loop structure comprises the sequence SEQ ID NO: 7. In some embodiments, the loop structure consists essentially of the sequence SEQ ID NO: 7. In some embodiments, the loop structure consists of the sequence SEQ ID NO: 7.

In some embodiments, a ribosomal frameshift element used in a miRNA-dependent polynucleotide circuit described herein comprises the sequence GGUUUUUCAGACUCAAGGAGCGGCAGUGUCAUCAAUGGCUCAAACCCUACUGC CG (SEQ ID NO: 8), or a sequence having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 8.

In some embodiments, a ribosomal frameshift element used in a miRNA-dependent polynucleotide circuit described herein comprises the sequence GGUUUUUCAGACUCAAGGAGCGGCAGUGUCAUCAAUGGCUCAAACCCUACUGC CG (SEQ ID NO: 12), or a sequence having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 12.

In various embodiments, a ribosomal frameshift element used in a miRNA-dependent polynucleotide circuit described herein causes a −1 frameshift in the presence of the ribosomal frameshift stimulator.

In various embodiments, a polynucleotide molecule described herein further comprises a promoter, and expression of the ribosomal frameshift stimulator is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding the ribosomal frameshift stimulator and one or more MSE sequences, and another transcript comprises the ribosomal frameshift element and the nucleotide sequence encoding the target polypeptide. In various embodiments, a polynucleotide molecule described herein is structured as an alternatively spliced construct, wherein one transcript comprises the nucleotide sequence encoding the ribosomal frameshift stimulator and one or more MSE sequences, and another transcript does not comprise the nucleotide sequence encoding the ribosomal frameshift stimulator and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of a ribosomal frameshift element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of a ribosomal frameshift element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of the ribosomal frameshift stimulator, a polynucleotide molecule described herein may comprise a second promoter which controls expression of the target polypeptide. The polynucleotide may further comprise one or more additional combinations of a ribosomal frameshift element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of a ribosomal frameshift stimulator transcript coding sequence and a subsequent promoter. The one or more DNA insulator elements may enhance efficiency of termination of transcription of the ribosomal frameshift stimulator.

When multiple combinations of a ribosomal frameshift element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019); incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence GGGGTTGGGGTTGCGCCTTTTCCAAGGCAGCCCTGGGTTTGCGCAGGGACGCGG CTGCTCTGGGCGTGGTTCCGGGAAACGCAGCGGCGCCGACCCTGGGTCTCGCAC ATTCTTCACGTCCGTTCGCAGCGTCACCCGGATCTTCGCCGCTACCCTTGTGGGCC CCCCGGCGACGCTTCCTGCTCCGCCCCTAAGTCGGGAAGGTTCCTTGCGGTTCGC GGCGTGCCGGACGTGACAAACGGAAGCCGCACGTCTCACTAGTACCCTCGCAGA CGGACAGCGCCAGGGAGCAATGGCAGCGCGCCGACCGCGATGGGCTGTGGCCA ATAGCGGCTGCTCAGCAGGGCGCGCCGAGAGCAGCGGCCGGGAAGGGGCGGTG CGGGAGGCGGGGTGTGGGGCGGTAGTGTGGGCCCTGTTCCTGCCCGCGCGGTGT TCCGCATTCTGCAAGCCTCCGGAGCGCACGTCGGCAGTCGGCTCCCTCGTTGACC GAATCACCGACCTCTCTCCCCAG (SEQ ID NO: 13), or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence GGTAGGCGTGTACGGTGGGAGGCCTATATAAGCAGAGCT (SEQ ID NO: 14), or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence 5′-CTCCTCCTCCGGAGGGCGGGGTGGAGGGTCGGGGGCCCTCTCTCCCCTCTCCCCT CTCTCTCTCCCTCTCTCCCTCTCCCTCCTCTCTCCCTCTCCCTCTCCTCTCTCTCCTC TCTCTCTCTCCCTCTCTCCCTCTCCCTCTCTCTCTCTCTCTCCCTCTCCCTCTCTCTC TCCTCCCTCCTCTCTCTCTCCTCTCTCTCCTCTCTCTCCCTCTCTCTCTCTCTCTCTC TCCCTCTCTC-3′ (SEQ ID NO: 11), or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

In various embodiments, the nucleotide sequence encoding the target polypeptide is positioned in frame with the ribosomal frameshift element.

In various embodiments, the nucleotide sequence encoding the target polypeptide is positioned in-1 translation frame in relation to the ribosomal frameshift element.

In some embodiments where multiple combinations of a ribosomal frameshift element and a nucleotide sequence encoding a target polypeptide are included, the nucleotide sequence encoding each target polypeptide may be independently positioned in frame or in-1 translation frame in relation to its closest 5′ ribosomal frameshift element in the resulting mRNA.

In some embodiments where multiple combinations of a ribosomal frameshift element and a nucleotide sequence encoding a target polypeptide are used, the additional ribosomal frameshift element is positioned 5′ to the nucleotide sequence encoding the additional target polypeptide. Translation of the additional target polypeptide may be controlled by the additional ribosomal frameshift element and may require either the presence or absence of the ribosomal frameshift stimulator. The nucleotide sequence encoding the additional target polypeptide may be positioned in frame or in-1 translation frame in relation to the additional ribosomal frameshift element.

In some embodiments, the nucleotide sequence target polypeptide is codon optimized at the 5′ end such that when a frameshift is induced, the 5′ of the transcript contains one or more stop codons. This can minimize the expression of unwanted protein products.

In some embodiments, the nucleotide sequence encoding a ribosomal frameshift stimulator further encodes a degron sequence to facilitate the degradation of the ribosomal frameshift stimulator. A degron is a short amino acid motif that can target a protein for active degradation by the cell's quality-control machinery, typically by recruiting specific E3 ubiquitin ligases or proteases. Degrons may be appended to or embedded in ribosomal frameshift stimulator to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.”

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In one embodiment, an exemplary polynucleotide of the present disclosure comprises from 5′ to 3′:
a) a first promoter;
b) a 5′ donor splice site;
c) a nucleotide sequence encoding a ribosomal frameshift stimulator;
d) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
e) a 3′ splice acceptor site, and
f) a first ribosomal frameshift element, and
g) a nucleotide sequence encoding a first target polypeptide,
   wherein the first ribosomal frameshift element is positioned 5′ to the nucleotide sequence encoding the first target polypeptide,
   wherein translation of the first target polypeptide is controlled by the first ribosomal frameshift element and requires either the presence or absence of the ribosomal frameshift stimulator, and
   wherein the nucleotide sequence encoding the first target polypeptide is positioned in frame or in-1 translation frame in relation to the first ribosomal frameshift element.

In one embodiment, the polynucleotide further comprises 3′ to the nucleotide sequence encoding the first target polypeptide from 5′ to 3′:
h) a second promoter;
i) a second ribosomal frameshift element, and
j) a nucleotide sequence encoding a second target polypeptide,
   wherein the second ribosomal frameshift element is positioned 5′ to the nucleotide sequence encoding the second target polypeptide,
   wherein translation of the second target polypeptide is controlled by the second ribosomal frameshift element and requires either the presence or absence of the ribosomal frameshift stimulator, and
   wherein the nucleotide sequence encoding the second target polypeptide is positioned in frame or in-1 translation frame in relation to the second ribosomal frameshift element.

In one embodiment, the polynucleotide further comprises a DNA insulator element, wherein said DNA insulator element is positioned between the nucleotide sequence encoding the first target polypeptide and the second promoter, and wherein said DNA insulator element enhances efficiency of termination of transcription.

In one embodiment, the 5′ donor splice site comprises the sequence AG*GU(A/G)AGU or AG*GU(A/G)GAU, wherein * denotes the splice junction.

In one embodiment, the 3′ acceptor splice site comprises a branch point, a pyrimidine tract, and the sequence AG*G, wherein * denotes the splice junction.

In another embodiment, an exemplary polynucleotide of the present disclosure comprises from 5′ to 3′:
a) a first promoter;
b) a nucleotide sequence encoding a ribosomal frameshift stimulator;
c) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence,
d) a second promoter;
e) a first ribosomal frameshift element, and
f) a nucleotide sequence encoding a first target polypeptide,
   wherein the first ribosomal frameshift element is positioned 5′ to the nucleotide sequence encoding the first target polypeptide,
   wherein translation of the first target polypeptide is controlled by the first ribosomal frameshift element and requires either the presence or absence of the ribosomal frameshift stimulator, and
   wherein the nucleotide sequence encoding the first target polypeptide is positioned in frame or in-1 translation frame in relation to the first ribosomal frameshift element.

In one embodiment, the polynucleotide further comprises a DNA insulator element, wherein said DNA insulator element is positioned between the 3′UTR of the ribosomal frameshift stimulator transcript coding sequence and the second promoter, and wherein said DNA insulator element enhances efficiency of termination of transcription.

In one embodiment, the polynucleotide further comprises 3′ to the nucleotide sequence encoding the first target polypeptide from 5′ to 3′:
g) a third promoter;
h) a second ribosomal frameshift element, and
i) a nucleotide sequence encoding a second target polypeptide,
   wherein the second ribosomal frameshift element is positioned 5′ to the nucleotide sequence encoding the second target polypeptide,
   wherein translation of the second target polypeptide is controlled by the second ribosomal frameshift element and requires either the presence or absence of the ribosomal frameshift stimulator, and
   wherein the nucleotide sequence encoding the second target polypeptide is positioned in frame or in-1 translation frame in relation to the second ribosomal frameshift element.

In one embodiment, an exemplary polynucleotide of the present disclosure comprises from 5′ to 3′:
a) one or more microRNA (miRNA) Silencing Element (MSE) sequences, wherein each of said one or more MSE sequences is targeted by a miRNA,
b) a nucleotide sequence encoding a ribosomal frameshift stimulator, wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
c) a bi-directional promoter;
d) a first ribosomal frameshift element;
e) a nucleotide sequence encoding a first target polypeptide, wherein the first ribosomal frameshift element is positioned 5′ to the nucleotide sequence encoding the first target polypeptide, wherein translation of the first target polypeptide is controlled by the first ribosomal frameshift element and requires either the presence or absence of the ribosomal frameshift stimulator, and wherein the bi-directional promoter drives expression of the ribosomal frameshift stimulator in one direction and the first target polypeptide in the opposite direction; and
f) optionally one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.
   Iron Response Element (IRE)-Based miRNA-Dependent Circuits

In some embodiments, the present disclosure provides a polynucleotide comprising: a) a nucleotide sequence encoding an iron regulatory protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence; c) a first iron response element; d) a nucleotide sequence encoding a first target polypeptide, wherein the first iron response element is positioned with respect to the nucleotide sequence encoding the first target polypeptide to allow translation of the first target polypeptide in the absence of the iron regulatory protein. Optionally, the polynucleotides further comprises one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In one embodiment, the present disclosure provides a system comprising: (i) a first polynucleotide comprising a) a nucleotide sequence encoding an iron regulatory protein; and b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence; and (ii) a second polynucleotide comprising c) a first iron response element; and d) a nucleotide sequence encoding a first target polypeptide, wherein the first iron response element is positioned with respect to the nucleotide sequence encoding the first target polypeptide to allow translation of the first target polypeptide in the absence of the iron regulatory protein. Optionally, the system further comprises (as part of the first polynucleotide, part of the second polynucleotide or as one or more additional polynucleotides) one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In various embodiments, a polynucleotide molecule described herein comprises a nucleotide sequence encoding an iron regulatory protein. The one or more MSE sequences are positioned with respect to the nucleotide sequence encoding the iron regulatory protein to allow expression of the iron regulatory protein in the absence of the respective miRNA targeting the MSE sequence.

In some embodiments, the one or more MSE sequences are positioned in the open reading frame (ORF) or in 3′ untranslated region (3′ UTR) of the iron regulatory protein transcript coding sequence.

In some embodiments, a miRNA-dependent polynucleotide circuits described herein utilize an iron response element (IRE). The IRE-based polynucleotide circuits described herein can act as molecular switches that can be activated exclusively within specific cell types. The IRE-based miRNA-dependent polynucleotide circuit, also termed “Specific microRNA-Targeted Autonomous Response (STAR) circuit” herein, can be seamlessly integrated into any viral vector to enable cell-specific delivery. This approach can use an endogenous host protein (e.g., iron regulatory protein) to minimize cytotoxicity and immunogenicity. This approach can block ribosomal translation of a target mRNA instead of inducing a frameshift.

Iron homeostasis in mammalian cells is tightly regulated to ensure that sufficient iron is available for essential biochemical processes while preventing toxicity from excess iron. Two key regulators of this system are Iron Regulatory Protein 1 (IRP1, also known as ACO1) and Iron Regulatory Protein 2 (IRP2, also known as IREB2). These proteins function as RNA-binding factors that post-transcriptionally regulate genes involved in iron uptake, storage, and utilization by interacting with IREs found in the untranslated regions (UTRs) of these mRNAs.

In some embodiments, the STAR circuit employs the host IRP2, or a modified form of IRP2, which represses translation of transcripts containing IRE in their 5′ UTR. By coupling miRNA-mediated silencing of IRP2 with a therapeutic cargo containing an IRE, it is demonstrated herein that cargo expression can be restricted to cells with a designated miRNA environment. The STAR circuit can leverage established miRNA expression patterns and their silencing potential as environmental cues, enabling dynamic, autonomous adjustments of cargo expression for precise, multilayered therapeutic control. In non-target cells lacking the designated miRNA, IRP2 is expressed, which can prevent cargo translation. In contrast, in cells where the miRNA is present, IRP2 can be silenced by fully complementary miRNA targets sites embedded in the 3′ UTR, enabling the expression of the IRE-containing cargo to that desired cellular environment. The present disclosure presents proof-of-concept data (in the Examples section below) for a viral-based delivery platform featuring diverse STAR circuits designed to respond to distinct cell-associated miRNAs, demonstrating that an IRE-containing cargo is selectively expressed in the desired target cells. Since most cell types express at least one miRNA that is either highly enriched or uniquely present, this programmable switch allows for the development of vectors that restrict gene expression to the desired cell type(s). These findings establish the STAR circuit as a versatile, programmable tool that can successfully mitigate risks associated with off-target expression, offering a scalable solution for future therapeutic applications across a range of genetic diseases.

Iron response elements (IREs) are known to have highly conserved sequences and structures. An IRE generally comprises a stem-loop element formed by a five base pair stem carrying a 6-nucleotide (nt) apical loop motif 5′CAGWGH-3′ (W=A or U and H=A, C, or U) separated from a lower stem of variable length by an internal loop or bulge containing a conserved C (Hentze et al., Gene. 1988 Dec. 10; 72 (1-2): 201-8, incorporated herein by reference in its entirety).

In some embodiments, an iron response element used in a polynucleotide circuit described herein comprises a stem-loop element comprising a 6-nucleotide apical loop motif 5′-CAGWGH-3′ (W=A or U and H=A, C, or U) operably linked to a five base-pair stem.

In some embodiments, an iron response element used in a polynucleotide circuit described herein further comprises an internal bulge containing a conserved cytosine (C) operably linked to the five base-pair stem.

In some embodiments, an iron response element used in a polynucleotide circuit described herein comprises an iron response element found in the 5′ UTR of the human ferritin transcript, hypoxia inducible factor 1 subunit alpha (HIF1A) transcript, or erythroid 5-aminolevulinate synthase (eALAS) transcript, or a functional variant or ortholog thereof.

In some embodiments, an iron response element used in a polynucleotide circuit described herein comprises the nucleotide sequence of any one of SEQ ID Nos: 32, 33, or 34, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID Nos: 32, 33, or 34.

In some embodiments, an iron response element used in a polynucleotide circuit described herein comprises an iron response element found in the 5′ UTR of the human ferritin transcript, or a functional variant or ortholog thereof. In some embodiments, an iron response element used in a polynucleotide circuit described herein comprises the nucleotide sequence of SEQ ID No: 32, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID No: 32. In some embodiments, an iron response element used in a polynucleotide circuit described herein comprises SEQ ID No: 32. In some embodiments, an iron response element used in a polynucleotide circuit described herein consists essentially of SEQ ID No: 32. In some embodiments, an iron response element used in a polynucleotide circuit described herein consists of SEQ ID No: 32.

In some embodiments, an iron response element used in a polynucleotide circuit described herein comprises an iron response element found in the 5′ UTR of the human HIF1A transcript, or a functional variant or ortholog thereof. In some embodiments, an iron response element used in a polynucleotide circuit described herein comprises the nucleotide sequence of SEQ ID No: 33, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID No: 33. In some embodiments, an iron response element used in a polynucleotide circuit described herein comprises SEQ ID No: 33. In some embodiments, an iron response element used in a polynucleotide circuit described herein consists essentially of SEQ ID No: 33. In some embodiments, an iron response element used in a polynucleotide circuit described herein consists of SEQ ID No: 33.

In some embodiments, an iron response element used in a polynucleotide circuit described herein comprises an iron response element found in the 5′ UTR of the human eALAS transcript, or a functional variant or ortholog thereof. In some embodiments, an iron response element used in a polynucleotide circuit described herein comprises the nucleotide sequence of SEQ ID No: 34, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID No: 34. In some embodiments, an iron response element used in a polynucleotide circuit described herein comprises SEQ ID No: 34. In some embodiments, an iron response element used in a polynucleotide circuit described herein consists essentially of SEQ ID No: 34. In some embodiments, an iron response element used in a polynucleotide circuit described herein consists of SEQ ID No: 34.

In some embodiments, the iron regulatory protein is Iron Regulatory Protein 2 (IRP2), Iron Regulatory Protein 1 (IRP1), or a functional variant or ortholog thereof.

Both IRP1 and IRP2 act as iron sensors, where their capacity to bind to IREs is suppressed in the presense of high intracellular iron levels. Their role is crucial for iron metabolism, influencing iron storage through ferritin, iron uptake via transferrin receptor 1 (TFRC), and iron transport via divalent metal transporter 1 (DMT1/SLC11A2). The interaction of IRPs with IREs allows them to regulate iron metabolism in a context-dependent manner. When IRPs bind to an IRE located in the 5′ UTR, such as in the ferritin and ferroportin mRNAs, they block translation initiation, preventing the synthesis of these iron-storage and export proteins. This differential regulation ensures that cells reduce iron storage and increase iron uptake when iron levels are low. Together, IRP1 and IRP2 share several similarities in function. Both act as post-transcriptional regulators of iron metabolism by binding to IREs, and both respond to fluctuations in intracellular iron levels. Under high iron conditions, their ability to bind IREs is reduced, allowing iron-related genes to be expressed as needed. Additionally, IRP1 and IRP2 exhibit similar binding affinities and specificities for IREs, enabling them to partially compensate for each other in maintaining iron homeostasis. Despite these similarities, IRP1 and IRP2 have key differences in structure and regulation. IRP1 has a dual functionality, existing in two distinct states depending on iron availability. Under iron-deficient conditions, IRP1 acts as an RNA-binding protein that regulates iron metabolism. However, when iron levels are high, IRP1 incorporates a 4Fe-4S cluster, converting into cytosolic aconitase, an enzyme involved in the TCA cycle. This conformational shift prevents IRP1 from binding to IREs, allowing ferritin and ferroportin expression to increase while reducing transferrin receptor 1 stability. This ability to function as both an iron sensor and a metabolic enzyme gives IRP1 a broader role beyond iron regulation. In contrast, IRP2 lacks aconitase activity and does not contain a 4Fe-4S cluster. Instead, it is more dedicated to iron sensing and regulation, as it is subject to rapid degradation under iron-replete conditions through ubiquitination and proteasomal degradation via the iron-dependent E3 ligase FBXL5.

In some embodiments, the iron regulatory protein used in a polynucleotide circuit described herein is not reactive to intracellular iron levels.

In some embodiments, the iron regulatory protein used in a polynucleotide circuit retains the ability to bind to an iron response element.

In some embodiments, the iron regulatory protein is human IRP1, or a functional variant or ortholog thereof.

In some embodiments, the human IRP1 comprises the amino acid sequence of SEQ ID NO: 15, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 15. In one embodiment, the IRP1 protein comprises the amino acid sequence of SEQ ID NO: 15. In one embodiment, the IRP1 protein consists essentially of the amino acid sequence of SEQ ID NO: 15. In one embodiment, the IRP1 protein consists of the amino acid sequence of SEQ ID NO: 15.

In some embodiments, the iron regulatory protein is human IRP2, or a functional variant or ortholog thereof.

In some embodiments, the human IRP2 comprises the amino acid sequence of SEQ ID NO: 16, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 16. In one embodiment, the IRP2 protein comprises the amino acid sequence of SEQ ID NO: 16. In one embodiment, the IRP2 protein consists essentially of the amino acid sequence of SEQ ID NO: 16. In one embodiment, the IRP2 protein consists of the amino acid sequence of SEQ ID NO: 16.

In some embodiments, the human IRP2 variant comprises the amino acids about 1-318 and about 443-750 of SEQ ID NO: 16. In some embodiments, the human IRP2 variant consists essentially of the amino acids about 1-318 and about 443-750 of SEQ ID NO: 16. In some embodiments, the human IRP2 variant consists of the amino acids about 1-318 and about 443-750 of SEQ ID NO: 16.

In some embodiments, the human IRP2 variant comprises about the N-terminal 750, 760, 770, 780, 790, 800, 810, 820, 830, 840, 850, 860, 870, 880, 890, 900, 910, 920, 930, 940, 950, or 960 amino acids of SEQ ID NO: 16. In some embodiments, the human IRP2 variant consists essentially of about the N-terminal 750, 760, 770, 780, 790, 800, 810, 820, 830, 840, 850, 860, 870, 880, 890, 900, 910, 920, 930, 940, 950, or 960 amino acids of SEQ ID NO: 16. In some embodiments, the human IRP2 variant consists of about the N-terminal 750, 760, 770, 780, 790, 800, 810, 820, 830, 840, 850, 860, 870, 880, 890, 900, 910, 920, 930, 940, 950, or 960 amino acids of SEQ ID NO: 16.

In some embodiments, the human IRP2 variant comprises about the N-terminal 750, 800, 850, 900, or 950 amino acids of SEQ ID NO: 16. In some embodiments, the human IRP2 variant consists essentially of the amino acids about the N-terminal 750, 800, 850, 900, or 950 amino acids of SEQ ID NO: 16. In some embodiments, the human IRP2 variant consists of the amino acids about the N-terminal 750, 800, 850, 900, or 950 amino acids of SEQ ID NO: 16.

In some embodiments, the human IRP2 variant lacks about the C-terminal 10, 20, 30, 40, 50, 60, 70, 80, 90, 100, 110, 120, 130, 140, 150, 160, 170, 180, 190, 200, or 210 amino acids of SEQ ID NO: 16. In some embodiments, the human IRP2 variant lacks about the C-terminal 13, 63, 113, 163, or 213 amino acids of SEQ ID NO: 16.

In some embodiments, the human IRP2 variant lacks a fragment within the C-terminal 213 amino acids of SEQ ID NO: 16. In some embodiments, the human IRP2 variant lacks a fragment of about 10, 20, 30, 40, 50, 60, 70, 80, 90, 100, 110, 120, 130, 140, 150, 160, 170, 180, 190, 200, or 210 amino acids within the C-terminal 213 amino acids of SEQ ID NO: 16.

In some embodiments, the human IRP2 variant comprises the amino acid sequence of any one of SEQ ID NOs: 24, 25, 26, or 27, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to any one of SEQ ID NOs: 24, 25, 26, or 27. In some embodiments, the human IRP2 variant comprises the amino acid sequence of any one of SEQ ID NOs: 24, 25, 26, or 27. In some embodiments, the human IRP2 variant consists essentially of the amino acid sequence of any one of SEQ ID NOs: 24, 25, 26, or 27. In some embodiments, the human IRP2 variant consists of the amino acid sequence of any one of SEQ ID NOs: 24, 25, 26, or 27.

In some embodiments, the human IRP2 variant lacks a fragment within the amino acids 319-442 of SEQ ID NO: 16. In some embodiments, the human IRP2 variant lacks a fragment of about 10, 20, 30, 40, 50, 60, 70, 80, 90, 100, 110, or 120 within the amino acids 319-442 of SEQ ID NO: 16. In some embodiments, the human IRP2 variant lacks the amino acids 319-442 of SEQ ID NO: 16.

In some embodiments, the human IRP2 variant comprises the amino acid sequence of SEQ ID NO: 21, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 21. In one embodiment, the IRP2 variant comprises the amino acid sequence of SEQ ID NO: 21. In one embodiment, the IRP2 variant consists essentially of the amino acid sequence of SEQ ID NO: 21. In one embodiment, the IRP2 variant consists of the amino acid sequence of SEQ ID NO: 21.

In some embodiments, the human IRP2 variant is not responsive to intracellular iron levels. In some embodiments, the human IRP2 variant is only partially responsive to intracellular iron levels.

In some embodiments, the human IRP2 variant comprises a substitution at one or more positions selected from cysteine 120, cysteine 375, cysteine 578, or cysteine 581 of SEQ ID NO: 16. The substitution may be any non-cysteine residue (e.g., cysteine or alanine) that does not significantly alter the folding properties of the wild type IRP2 protein. In some embodiments, the human IRP2 variant comprises a serine substitution at one or more positions selected from cysteine 120, cysteine 375, cysteine 578, or cysteine 581.

In some embodiments, the human IRP2 variant comprises a substitution at cysteine 578 of SEQ ID NO: 16. In some embodiments, the human IRP2 variant comprises a serine substitution at cysteine 578 of SEQ ID NO: 16.

In some embodiments, the human IRP2 variant comprises a substitution at cysteine 581 of SEQ ID NO: 16. In some embodiments, the human IRP2 variant comprises a serine substitution at cysteine 581 of SEQ ID NO: 16.

In some embodiments, the human IRP2 variant comprises a substitution at cysteine 578 and cysteine 581 of SEQ ID NO: 16. In some embodiments, the human IRP2 variant comprises a serine substitution at cysteine 578 and cysteine 581.

In some embodiments, the human IRP2 variant comprises the amino acid sequence of SEQ ID NO: 30, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 30. In one embodiment, the IRP2 variant comprises the amino acid sequence of SEQ ID NO: 30. In one embodiment, the IRP2 variant consists essentially of the amino acid sequence of SEQ ID NO: 30. In one embodiment, the IRP2 variant consists of the amino acid sequence of SEQ ID NO: 30.

In various embodiments, a polynucleotide molecule described herein further comprises a promoter, and expression of the iron regulatory protein is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding the iron regulatory protein and one or more MSE sequences, and another transcript comprises the iron response element and the nucleotide sequence encoding the target polypeptide. In various embodiments, a polynucleotide molecule described herein is structured as an alternatively spliced construct, wherein one transcript comprises the nucleotide sequence encoding the iron regulatory protein and one or more MSE sequences, and another transcript does not comprise the nucleotide sequence encoding the iron regulatory protein and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of an iron response element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of an iron response element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of the iron regulatory protein, a polynucleotide molecule described herein may comprise a second promoter which controls expression of the target polypeptide. The polynucleotide may further comprise one or more additional combinations of an iron response element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of an iron regulatory protein transcript coding sequence and a subsequent promoter. The one or more DNA insulator elements may enhance efficiency of termination of transcription of the iron regulatory protein.

When multiple combinations of an iron response element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019); incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence SEQ ID NO: 13, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence SEQ ID NO: 14, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence SEQ ID NO: 11, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

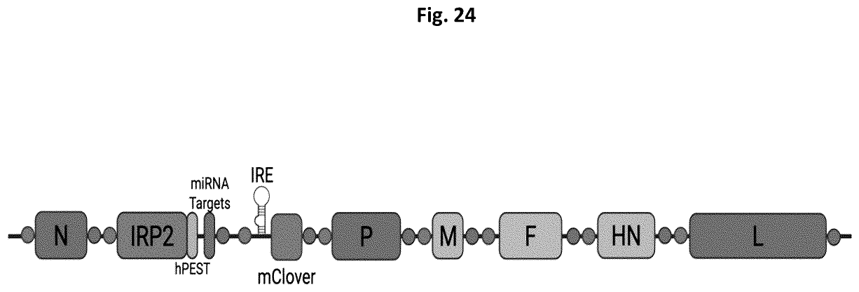

In some embodiments, the nucleotide sequence encoding an iron regulatory protein further encodes a degron sequence to facilitate the degradation of the iron regulatory protein. Degrons may be appended to or embedded in the iron regulatory protein to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.” An exemplary design of a miRNA-dependent polynucleotide circuit including a degron is shown in FIG. 24.

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In one embodiment, a polynucleotide circuit described herein encodes a fusion protein comprising an iron regulatory protein fused to a degron sequence, wherein the fusion protein comprises the amino acid sequence of SEQ ID NO: 19, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 19.

In one embodiment, an exemplary polynucleotide comprises from 5′ to 3′:
a) a first promoter;
b) a 5′ donor splice site;
c) a nucleotide sequence encoding an iron regulatory protein;
d) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence;
e) a 3′ splice acceptor site;
f) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
g) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In one embodiment, an exemplary polynucleotide comprises from 5′ to 3′:
a) a first promoter;
b) a nucleotide sequence encoding an iron regulatory protein;
c) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence,
d) a second promoter;
e) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
f) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

In one embodiment, an exemplary polynucleotide comprises from 5′ to 3′: a first promoter;
a) one or more microRNA (miRNA) Silencing Element (MSE) sequences, wherein each of said one or more MSE sequences is targeted by a miRNA;
b) a nucleotide sequence encoding an iron regulatory protein, wherein each of said one or more MSE sequences is positioned with respect to the nucleotide sequence encoding the iron regulatory protein to allow expression of the iron regulatory protein in the absence of the respective miRNA targeting said MSE sequence;
c) a bi-directional promoter;
d) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
e) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.
   Lin28-Based miRNA-Dependent Circuits

In some embodiments, the present disclosure provides a polynucleotide comprising: a) a nucleotide sequence encoding Lin28; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding Lin28 to allow expression of Lin28 in the absence of the respective miRNA targeting said MSE sequence; c) a first pre-let-7 element; and d) a nucleotide sequence encoding a first target polypeptide, wherein the first pre-let-7 element is positioned with respect to the nucleotide sequence encoding the first target polypeptide to allow translation of the first target polypeptide in the absence of Lin28. Optionally, the polynucleotides further comprises one or more additional combinations of a pre-let-7 element and a nucleotide sequence encoding a target polypeptide, wherein within each combination, the pre-let-7 element is positioned with respect to the nucleotide sequence encoding the target polypeptide to allow translation of the target polypeptide in the absence of Lin28, wherein translation of each additional target polypeptide is controlled by its closest pre-let-7 element in the resulting mRNA.

In one embodiment, the present disclosure provides a system comprising: (i) a first polypeptide comprising a) a nucleotide sequence encoding Lin28; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding Lin28 to allow expression of Lin28 in the absence of the respective miRNA targeting said MSE sequence; and (ii) a second polynucleotide comprising c) a first pre-let-7 element; and d) a nucleotide sequence encoding a first target polypeptide, wherein the first pre-let-7 element is positioned with respect to the nucleotide sequence encoding the first target polypeptide to allow translation of the first target polypeptide in the absence of Lin28. Optionally, the system further comprises (as part of the first polynucleotide, part of the second polynucleotide or as one or more additional polynucleotides) one or more additional combinations of a pre-let-7 element and a nucleotide sequence encoding a target polypeptide, wherein within each combination, the pre-let-7 element is positioned with respect to the nucleotide sequence encoding the target polypeptide to allow translation of the target polypeptide in the absence of Lin28, wherein translation of each additional target polypeptide is controlled by its closest pre-let-7 element in the resulting mRNA.

In various embodiments, a polynucleotide molecule described herein comprises a nucleotide sequence encoding Lin28, or a functional variant or ortholog thereof. The one or more MSE sequences are positioned with respect to the nucleotide sequence encoding Lin28 to allow expression of Lin28 in the absence of the respective miRNA targeting the MSE sequence.

In some embodiments, the one or more MSE sequences are positioned in the open reading frame (ORF) or in 3′ untranslated region (3′ UTR) of Lin28 transcript coding sequence.

In various embodiments, a polynucleotide molecule described herein further comprises a promoter, and expression of Lin28 is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding Lin28 and one or more MSE sequences, and another transcript comprises the pre-let-7 element and the nucleotide sequence encoding the target polypeptide. In various embodiments, a polynucleotide molecule described herein is structured as an alternatively spliced construct, wherein one transcript comprises the nucleotide sequence encoding Lin28 and one or more MSE sequences, and another transcript does not comprise the nucleotide sequence encoding the Lin28 and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of a pre-let-7 element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of a pre-let-7 element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of Lin28, a polynucleotide molecule described herein may comprise a second promoter which controls expression of the target polypeptide. The polynucleotide may further comprise one or more additional combinations of a pre-let-7 element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of an Lin28 transcript coding sequence and a subsequent promoter. The one or more DNA insulator elements may enhance efficiency of termination of transcription of Lin28.

When multiple combinations of a pre-let-7 element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019); incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence SEQ ID NO: 13, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence SEQ ID NO: 14, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence SEQ ID NO: 11, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

In some embodiments, the nucleotide sequence encoding Lin28 further encodes a degron sequence to facilitate the degradation of Lin28. Degrons may be appended to or embedded in the Lin28 protein to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.”

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In various embodiments, Lin28 comprises the amino acid sequence of SEQ ID NO: 35, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 35. In one embodiment, Lin28 comprises the amino acid sequence of SEQ ID NO: 35. In one embodiment, Lin28 consists essentially of the amino acid sequence of SEQ ID NO: 35. In one embodiment, Lin28 consists of the amino acid sequence of SEQ ID NO: 35.

In some embodiments, the first pre-let-7 element is positioned in the 5′ UTR or the 3′ UTR of the nucleotide sequence encoding a first target polypeptide.

In some embodiments, when one or more additional combinations of a pre-let-7 element and a nucleotide sequence encoding a target polypeptide are present, each additional pre-let-7 element is positioned independently in the 5′ UTR or the 3′ UTR of the nucleotide sequence encoding the respective additional target polypeptide.

In some embodiments, the pre-let-7 element comprises a tetranucleotide motif 5′-GGAG-3′.

In some embodiments, the pre-let-7 element comprises the nucleotide sequence of SEQ ID No: 36, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO:

36. In one embodiment, the pre-let-7 element comprises the nucleotide sequence of SEQ ID NO: 36. In one embodiment, the pre-let-7 element consists essentially of the nucleotide sequence of SEQ ID NO: 36. In one embodiment, the pre-let-7 element consists of the nucleotide sequence of SEQ ID NO: 36.

MS2-Based miRNA-Dependent Circuits

In some embodiments, the present disclosure provides a polynucleotide comprising: a) a nucleotide sequence encoding an MS2 bacteriophage coat protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the MS2 bacteriophage coat protein to allow expression of the MS2 bacteriophage coat protein in the absence of the respective miRNA targeting said MSE sequence; c) a first MS2 responsive RNA element; and d) a nucleotide sequence encoding a first target polypeptide, wherein the first MS2 responsive RNA element is positioned with respect to the nucleotide sequence encoding the first target polypeptide to allow translation of the first target polypeptide in the absence of the MS2 bacteriophage coat protein. Optionally, the polynucleotides further comprises one or more additional combinations of an MS2 responsive RNA element and a nucleotide sequence encoding a target polypeptide, wherein within each combination, the MS2 responsive RNA element is positioned with respect to the nucleotide sequence encoding the target polypeptide to allow translation of the target polypeptide in the absence of the MS2 bacteriophage coat protein, wherein translation of each additional target polypeptide is controlled by its closest MS2 responsive RNA element in the resulting mRNA.

In one embodiment, the present disclosure provides a system comprising: (i) a first polypeptide comprising a) a nucleotide sequence encoding an MS2 bacteriophage coat protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the MS2 bacteriophage coat protein to allow expression of the MS2 bacteriophage coat protein in the absence of the respective miRNA targeting said MSE sequence; and (ii) a second polynucleotide comprising c) a first MS2 responsive RNA element; and d) a nucleotide sequence encoding a first target polypeptide, wherein the first MS2 responsive RNA element is positioned with respect to the nucleotide sequence encoding the first target polypeptide to allow translation of the first target polypeptide in the absence of the MS2 bacteriophage coat protein. Optionally, the system further comprises (as part of the first polynucleotide, part of the second polynucleotide or as one or more additional polynucleotides) one or more additional combinations of an MS2 responsive RNA element and a nucleotide sequence encoding a target polypeptide, wherein within each combination, the MS2 responsive RNA element is positioned with respect to the nucleotide sequence encoding the target polypeptide to allow translation of the target polypeptide in the absence of the MS2 bacteriophage coat protein, wherein translation of each additional target polypeptide is controlled by its closest MS2 responsive RNA element in the resulting mRNA.

In various embodiments, a polynucleotide molecule described herein comprises a nucleotide sequence encoding the MS2 bacteriophage coat protein, or a functional variant or ortholog thereof. The one or more MSE sequences are positioned with respect to the nucleotide sequence encoding the MS2 bacteriophage coat protein to allow expression of the MS2 bacteriophage coat protein in the absence of the respective miRNA targeting the MSE sequence.

In some embodiments, the one or more MSE sequences are positioned in the open reading frame (ORF) or in 3′ untranslated region (3′ UTR) of the MS2 bacteriophage coat protein transcript coding sequence.

In various embodiments, a polynucleotide molecule described herein further comprises a promoter, and expression of the MS2 bacteriophage coat protein is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding the MS2 bacteriophage coat protein and one or more MSE sequences, and another transcript comprises the MS2 responsive RNA element and the nucleotide sequence encoding the target polypeptide. In various embodiments, a polynucleotide molecule described herein is structured as an alternatively spliced construct, wherein one transcript comprises the nucleotide sequence encoding the MS2 bacteriophage coat protein and one or more MSE sequences, and another transcript does not comprise the nucleotide sequence encoding the MS2 bacteriophage coat protein and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of a MS2 responsive RNA element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of a MS2 responsive RNA element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of the MS2 bacteriophage coat protein, a polynucleotide molecule described herein may comprise a second promoter which controls expression of the target polypeptide. The polynucleotide may further comprise one or more additional combinations of a MS2 responsive RNA element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of an the MS2 bacteriophage coat protein transcript coding sequence and a subsequent promoter. The one or more DNA insulator elements may enhance efficiency of termination of transcription of the MS2 bacteriophage coat protein.

When multiple combinations of a MS2 responsive RNA element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019); incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence SEQ ID NO: 13, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence SEQ ID NO: 14, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence SEQ ID NO: 11, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

In some embodiments, the nucleotide sequence encoding the MS2 bacteriophage coat protein further encodes a degron sequence to facilitate the degradation of the MS2 bacteriophage coat protein. Degrons may be appended to or embedded in the MS2 bacteriophage coat protein to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.”

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In various embodiments, the MS2 bacteriophage coat protein comprises the amino acid sequence of SEQ ID NO: 37, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 37. In one embodiment, the MS2 bacteriophage coat protein comprises the amino acid sequence of SEQ ID NO: 37. In one embodiment, the MS2 bacteriophage coat protein consists essentially of the amino acid sequence of SEQ ID NO:

37. In one embodiment, the MS2 bacteriophage coat protein consists of the amino acid sequence of SEQ ID NO: 37.

In some embodiments, the first MS2 responsive RNA element is positioned in the 5′ UTR or the 3′ UTR of the nucleotide sequence encoding a first target polypeptide.

In some embodiments, when one or more additional combinations of a MS2 responsive RNA element and a nucleotide sequence encoding a target polypeptide are present, each additional MS2 responsive RNA element is positioned independently in the 5′ UTR or the 3′ UTR of the nucleotide sequence encoding the respective additional target polypeptide.

In some embodiments, the MS2 responsive RNA element comprises the nucleotide sequence of SEQ ID No: 38, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 38. In one embodiment, the MS2 responsive RNA element comprises the nucleotide sequence of SEQ ID NO: 38. In one embodiment, the MS2 responsive RNA element consists essentially of the nucleotide sequence of SEQ ID NO: 38. In one embodiment, the MS2 responsive RNA element consists of the nucleotide sequence of SEQ ID NO: 38.

PP7-Based miRNA-Dependent Circuits

In some embodiments, the present disclosure provides a polynucleotide comprising: a) a nucleotide sequence encoding a PP7 bacteriophage coat protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the PP7 bacteriophage coat protein to allow expression of the PP7 bacteriophage coat protein in the absence of the respective miRNA targeting said MSE sequence; c) a first PP7 responsive RNA element; and d) a nucleotide sequence encoding a first target polypeptide, wherein the first PP7 responsive RNA element is positioned with respect to the nucleotide sequence encoding the first target polypeptide to allow translation of the first target polypeptide in the absence of the PP7 bacteriophage coat protein. Optionally, the polynucleotides further comprises one or more additional combinations of a PP7 responsive RNA element and a nucleotide sequence encoding a target polypeptide, wherein within each combination, the PP7 responsive RNA element is positioned with respect to the nucleotide sequence encoding the target polypeptide to allow translation of the target polypeptide in the absence of the PP7 bacteriophage coat protein, wherein translation of each additional target polypeptide is controlled by its closest PP7 responsive RNA element in the resulting mRNA.

In one embodiment, the present disclosure provides a system comprising: (i) a first polypeptide comprising a) a nucleotide sequence encoding a PP7 bacteriophage coat protein; b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the PP7 bacteriophage coat protein to allow expression of the PP7 bacteriophage coat protein in the absence of the respective miRNA targeting said MSE sequence; and (ii) a second polynucleotide comprising c) a first PP7 responsive RNA element; and d) a nucleotide sequence encoding a first target polypeptide, wherein the first PP7 responsive RNA element is positioned with respect to the nucleotide sequence encoding the first target polypeptide to allow translation of the first target polypeptide in the absence of the PP7 bacteriophage coat protein. Optionally, the system further comprises (as part of the first polynucleotide, part of the second polynucleotide or as one or more additional polynucleotides) one or more additional combinations of a PP7 responsive RNA element and a nucleotide sequence encoding a target polypeptide, wherein within each combination, the PP7 responsive RNA element is positioned with respect to the nucleotide sequence encoding the target polypeptide to allow translation of the target polypeptide in the absence of the PP7 bacteriophage coat protein, wherein translation of each additional target polypeptide is controlled by its closest PP7 responsive RNA element in the resulting mRNA.

In various embodiments, a polynucleotide molecule described herein comprises a nucleotide sequence encoding the PP7 bacteriophage coat protein, or a functional variant or ortholog thereof. The one or more MSE sequences are positioned with respect to the nucleotide sequence encoding the PP7 bacteriophage coat protein to allow expression of the PP7 bacteriophage coat protein in the absence of the respective miRNA targeting the MSE sequence.

In some embodiments, the one or more MSE sequences are positioned in the open reading frame (ORF) or in 3′ untranslated region (3′ UTR) of the PP7 bacteriophage coat protein transcript coding sequence.

In various embodiments, a polynucleotide molecule described herein further comprises a promoter, and expression of the PP7 bacteriophage coat protein is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding the PP7 bacteriophage coat protein and one or more MSE sequences, and another transcript comprises the PP7 responsive RNA element and the nucleotide sequence encoding the target polypeptide. In various embodiments, a polynucleotide molecule described herein is structured as an alternatively spliced construct, wherein one transcript comprises the nucleotide sequence encoding the PP7 bacteriophage coat protein and one or more MSE sequences, and another transcript does not comprise the nucleotide sequence encoding the PP7 bacteriophage coat protein and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of a PP7 responsive RNA element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of a PP7 responsive RNA element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of the PP7 bacteriophage coat protein, a polynucleotide molecule described herein may comprise a second promoter which controls expression of the target polypeptide. The polynucleotide may further comprise one or more additional combinations of a PP7 responsive RNA element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of an the PP7 bacteriophage coat protein transcript coding sequence and a subsequent promoter. The one or more DNA insulator elements may enhance efficiency of termination of transcription of the PP7 bacteriophage coat protein.

When multiple combinations of a PP7 responsive RNA element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019);

incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence SEQ ID NO: 13, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence SEQ ID NO: 14, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence SEQ ID NO: 11, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

In some embodiments, the nucleotide sequence encoding the PP7 bacteriophage coat protein further encodes a degron sequence to facilitate the degradation of the PP7 bacteriophage coat protein. Degrons may be appended to or embedded in the PP7 bacteriophage coat protein to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.”

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In various embodiments, the PP7 bacteriophage coat protein comprises the amino acid sequence of SEQ ID NO: 39, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 39. In one embodiment, the PP7 bacteriophage coat protein comprises the amino acid sequence of SEQ ID NO: 39. In one embodiment, the PP7 bacteriophage coat protein consists essentially of the amino acid sequence of SEQ ID NO: 39. In one embodiment, the PP7 bacteriophage coat protein consists of the amino acid sequence of SEQ ID NO: 39.

In some embodiments, the first PP7 responsive RNA element is positioned in the 5′ UTR or the 3′ UTR of the nucleotide sequence encoding a first target polypeptide.

In some embodiments, when one or more additional combinations of a PP7 responsive RNA element and a nucleotide sequence encoding a target polypeptide are present, each additional PP7 responsive RNA element is positioned independently in the 5′ UTR or the 3′ UTR of the nucleotide sequence encoding the respective additional target polypeptide.

In some embodiments, the PP7 responsive RNA element comprises the nucleotide sequence of SEQ ID No: 40, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 40. In one embodiment, the PP7 responsive RNA element comprises the nucleotide sequence of SEQ ID NO: 40. In one embodiment, the PP7 responsive RNA element consists essentially of the nucleotide sequence of SEQ ID NO: 40. In one embodiment, the PP7 responsive RNA element consists of the nucleotide sequence of SEQ ID NO: 40.

U1A-Based miRNA-Dependent Circuits

In some embodiments, the present disclosure provides a polynucleotide comprising: a) a nucleotide sequence encoding a U1 small nuclear ribonucleoprotein A (U1A); b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the U1A to allow expression of the U1A in the absence of the respective miRNA targeting said MSE sequence; c) a first U1A responsive RNA element; and d) a nucleotide sequence encoding a first target polypeptide, wherein the first U1A responsive RNA element is positioned with respect to the nucleotide sequence encoding the first target polypeptide to allow translation of the first target polypeptide in the absence of the U1A. Optionally, the polynucleotides further comprises one or more additional combinations of a U1A responsive RNA element and a nucleotide sequence encoding a target polypeptide, wherein within each combination, the U1A responsive RNA element is positioned with respect to the nucleotide sequence encoding the target polypeptide to allow translation of the target polypeptide in the absence of the U1A, wherein translation of each additional target polypeptide is controlled by its closest U1A responsive RNA element in the resulting mRNA.

In one embodiment, the present disclosure provides a system comprising: (i) a first polypeptide comprising a) a nucleotide sequence encoding a U1 small nuclear ribonucleoprotein A (U1A); b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the U1A to allow expression of the U1A in the absence of the respective miRNA targeting said MSE sequence; and (ii) a second polynucleotide comprising c) a first U1A responsive RNA element; and d) a nucleotide sequence encoding a first target polypeptide, wherein the first U1A responsive RNA element is positioned with respect to the nucleotide sequence encoding the first target polypeptide to allow translation of the first target polypeptide in the absence of the U1A. Optionally, the system further comprises (as part of the first polynucleotide, part of the second polynucleotide or as one or more additional polynucleotides) one or more additional combinations of a U1A responsive RNA element and a nucleotide sequence encoding a target polypeptide, wherein within each combination, the U1A responsive RNA element is positioned with respect to the nucleotide sequence encoding the target polypeptide to allow translation of the target polypeptide in the absence of the U1A, wherein translation of each additional target polypeptide is controlled by its closest U1A responsive RNA element in the resulting mRNA.

In various embodiments, a polynucleotide molecule described herein comprises a nucleotide sequence encoding the U1A, or a functional variant or ortholog thereof. The one or more MSE sequences are positioned with respect to the nucleotide sequence encoding the U1A to allow expression of the U1A in the absence of the respective miRNA targeting the MSE sequence.

In some embodiments, the one or more MSE sequences are positioned in the open reading frame (ORF) or in 3′ untranslated region (3′ UTR) of the U1A transcript coding sequence.

In various embodiments, a polynucleotide molecule described herein further comprises a promoter, and expression of the U1A is under the control of the promoter. In some embodiments, expression of the target polypeptide is under the control of the same promoter.

In various embodiments, a polynucleotide molecule described herein is structured as an alternatively spliced construct, wherein one transcript comprises the nucleotide sequence encoding the U1A and one or more MSE sequences, and another transcript comprises the U1A responsive RNA element and the nucleotide sequence encoding the target polypeptide. In various embodiments, a polynucleotide molecule described herein is structured as an alternatively spliced construct, wherein one transcript comprises the nucleotide sequence encoding the U1A and one or more MSE sequences, and another transcript does not comprise the nucleotide sequence encoding the U1A and one or more MSE sequence.

When the miRNA-dependent polynucleotide circuit is structured as an alternatively spliced construct, the miRNA-dependent polynucleotide circuit further comprises a 5′ donor splice site and a 3′ acceptor splice site. In one embodiment, the 5′ donor splice site comprises the sequence AG*GU(A/G)AGU, wherein * denotes the splice junction. In one embodiment, the 5′ donor splice site comprises the sequence AG*GU(A/G)GAU, wherein * denotes the splice junction. In one embodiment, the 3′ acceptor splice site comprises a branch point, a pyrimidine tract, and the sequence AG*G, wherein * denotes the splice junction.

In some embodiments, a polynucleotide molecule described herein may comprise one or more additional combinations of a U1A responsive RNA element and a nucleotide sequence encoding a target polypeptide, and expression of the first target polypeptide and the one or more additional target polypeptides is under the control of the same promoter. In such embodiments, the polynucleotide molecule may further comprise other elements such as a 2A ribosomal “stop-carry” site encoding a 2A peptide (e.g., porcine teschovirus-1 2A (P2A), Thosea asigna virus 2A (T2A), equine rhinitis A virus 2A (E2A), or foot-and-mouth disease virus 2A (F2A)) to generate a polycistronic mRNA.

In some embodiments, a polynucleotide molecule described herein may comprise one or more additional combinations of a U1A responsive RNA element and a nucleotide sequence encoding a target polypeptide, and expression of the first target polypeptide is under the control of a first promoter, and expression of each of the additional target polypeptides is under the control of respective additional promoters.

In some embodiments, in addition to the promoter which controls the expression of the U1A, a polynucleotide molecule described herein may comprise a second promoter which controls expression of the target polypeptide. The polynucleotide may further comprise one or more additional combinations of a U1A responsive RNA element and a nucleotide sequence encoding a target polypeptide, and expression of each of the additional target polypeptides is under the control of respective additional promoters.

In further embodiments, a polynucleotide molecule described herein may further comprise one or more DNA insulator elements. The one or more DNA insulator elements may be positioned between the 3′UTR of an the U1A transcript coding sequence and a subsequent promoter. The one or more DNA insulator elements may enhance efficiency of termination of transcription of the U1A.

When multiple combinations of a U1A responsive RNA element and a nucleotide sequence encoding a target polypeptide are included, the DNA insulator elements may be positioned between the 3′UTR of each subsequent transcript and each subsequent promoter, and the DNA insulator elements may enhance efficiency of termination of transcription.

Promotors used in a polynucleotide molecule described herein may be an RNA polymerase II promoter. Examples of RNA polymerase II promoter include, but are not limited to, a cytomegalovirus (CMV) promoter, a simian virus 40 (SV40) promoter, a human elongation factor 1 alpha (EF1α) promoter, or a phosphoglycerate kinase (PGK) promoter, a minimal promoter fragment derived from the CMV promoter (minCMV promoter), a RSV LTR, a MoMLV LTR, a CK6 promoter, a transthyretin promoter (TTR), a TK promoter, a tetracycline responsive promoter (TRE), an HBV promoter, an hAAT promoter, a LSP promoter, chimeric liver-specific promoters (LSPs), a E2F promoter, a telomerase (hTERT) promoter, a cytomegalovirus enhancer/chicken beta-actin/Rabbit β-globin promoter (CAG) promoter, a rod opsin promoter, a cone opsin promoter, a beta phosphodiesterase (PDE) promoter, a retinitis pigmentosa (RP1) promoter, or an interphotoreceptor retinoid-binding protein gene (IRBP) promoter.

In some embodiments, a promoter used in the present disclosure is one controlled by the polymerase of the viral vector used (e.g., Sendai).

Promotors used in a polynucleotide molecule described herein can be bi-directional promoters. Examples of bi-directional promoters include, but are not limited to, human promoter for 3-phosphoglycerate kinase (hPGK), a minimal Cytomegalovirus (CMV) promoter, TRE3G promoter (Heumüller, M. et al., Nat Methods 16, 699-702 (2019); incorporated herein by reference in its entirety), Ponasterone A promoter (Genes & Dev., 2013, 27:541-551; incorporated herein by reference in its entirety), or any combinations thereof.

In certain embodiments, a bi-direction promoter comprises a hPGK promoter in one direction and a minimal CMV promoter in the other direction. In some embodiments, a bi-directional promoter comprises a hPGK promoter comprising the nucleotide sequence SEQ ID NO: 13, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 13. In some embodiments, a bi-directional promoter comprises a minimal CMV promoter comprising the nucleotide sequence SEQ ID NO: 14, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 14.

In one embodiment, a DNA insulator element used herein comprises CCCTC-binding factor sites. For example, the DNA insulator may be derived from the chicken beta-globin gene and may comprise the nucleotide sequence SEQ ID NO: 11, or a variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 11.

In some embodiments, the nucleotide sequence encoding the U1A further encodes a degron sequence to facilitate the degradation of the U1A. Degrons may be appended to or embedded in the U1A to tune its half-life, enable conditional or ligand-induced degradation (e.g., auxin-inducible degron, Shield-1/DD systems), or create switch-like control of pathway activity. Example degron sequences that can be used in the present disclosure include N-degrons and C-degrons (recognized by the N-/C-degron pathways), PEST motifs, and cell-cycle “destruction boxes.”

In one embodiment, the degron sequence used herein is a PEST sequence. In one embodiment, the degron sequence used herein is a hPEST sequence optimized for use in human cells, and is rich in proline (P), glutamic acid (E), serine(S), and threonine (T).

In one embodiment, the degron sequence comprises the amino acid sequence of SEQ ID NO: 17, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 17.

In one embodiment, the nucleotide encoding a degron sequence comprises the nucleotide sequence of SEQ ID NO: 18, or a functional variant having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 18.

In various embodiments, the U1A comprises the amino acid sequence of SEQ ID NO: 41, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 41. In one embodiment, the U1A comprises the amino acid sequence of SEQ ID NO: 41. In one embodiment, the U1A consists essentially of the amino acid sequence of SEQ ID NO: 41. In one embodiment, the U1A consists of the amino acid sequence of SEQ ID NO: 41.

In some embodiments, the first U1A responsive RNA element is positioned in the 5′ UTR or the 3′ UTR of the nucleotide sequence encoding a first target polypeptide.

In some embodiments, when one or more additional combinations of a U1A responsive RNA element and a nucleotide sequence encoding a target polypeptide are present, each additional U1A responsive RNA element is positioned independently in the 5′ UTR or the 3′ UTR of the nucleotide sequence encoding the respective additional target polypeptide.

In some embodiments, the U1A responsive RNA element comprises the nucleotide sequence of SEQ ID No: 42, or a sequence having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity to SEQ ID NO: 42. In one embodiment, the U1A responsive RNA element comprises the nucleotide sequence of SEQ ID NO: 42. In one embodiment, the U1A responsive RNA element consists essentially of the nucleotide sequence of SEQ ID NO: 42. In one embodiment, the U1A responsive RNA element consists of the nucleotide sequence of SEQ ID NO: 42.

miRNAs and miRNA Silencing Elements

miRNAs are small (19-25 bp), non-coding RNA molecules encoded in the genomes of plants and animals. These highly conserved, short RNAs regulate the expression of genes by binding to specific mRNAs in a sequence-specific manner. Canonically, miRNA targeting is reliant on base pairing of the “seed” region which typically is comprised of nucleotides 1-7 or 2-8 of the miRNA to sites in the mRNA 3′ untranslated region (3′ UTR). Bartel, Cell 116 (2): 281 (2004). miRNA regulation moderately affects global protein production resulting in a “fine tuning” of the cellular transcriptome. Baek et al., Nature 455 (7209): 64 (2008) and Selbach et al., Nature 455 (7209): 58 (2008).

Like mRNAs, miRNA expression profiles appear to vary from tissue to tissue but are similar for identical tissues in different individuals (Baskerville and Bartel, 2005). Determining a miRNA with the desired expression profile may be achieved using techniques known to those skilled in the art. For example, the mir Vana™ miRNA Probe Set and mirVana™ miRNA Detection Kit available from Thermo Fisher Scientific. may be used to compare the miRNA expression profiles in human tissues according to the manufacturer's instructions. Another common way of identifying tissue-specific miRNAs is using Northern Blot. An example of such a technique is described in Lagos-Quintana M et al, Current Biol (2002) 12:735-739, which is incorporated herein by reference in its entirety. Methods of identifying new miRNAs and their target sequences using a computation approach are disclosed in WO2004/066183 and Brennecke J et al, PLOS Biology (2005) 3 (3): 0404-0418 (Brennecke et al., 2005), each of which is incorporated herein by reference in its entirety. Once the miRNA has been identified, one can generate an MSE sequence by generating a reverse and complement sequence of the miRNA. In most cases, a MSE sequence should have at least complementarity to the seed region of the miRNA and additional complementarity following the seed sequence.

Several hundred miRNAs have been cloned and sequenced from mouse, human, Drosphila, C. elegans and Arabidopsis. Examples of such sequences may be found on microRNA databases, e.g., miRBase (mirbase.org).

In various embodiments, a polynucleotide molecule of the present disclosure comprises one or more microRNA (miRNA) Silencing Element (MSE) sequences, wherein each of the one or more MSE sequences is targeted by a miRNA. In some embodiments, the polynucleotide comprises two MSE sequences. In some embodiments, the polynucleotide comprises three MSE sequences. In some embodiments, the polynucleotide comprises four MSE sequences. In some embodiments, the polynucleotide comprises five MSE sequences. In some embodiments, the polynucleotide comprises six MSE sequences. In some embodiments, the polynucleotide comprises seven MSE sequences. In some embodiments, the polynucleotide comprises eight MSE sequences. In some embodiments, the polynucleotide comprises nine MSE sequences. In some embodiments, the polynucleotide comprises ten or more MSE sequences.

In some embodiments, when two or more MSE sequences are present, at least two of these MSE sequences are targeted by the same miRNA. In some embodiments, when two or more MSE sequences are present, at least two of these MSE sequences are targeted by different miRNAs.

In various embodiments, the one or more MSE sequences are targeted by miRNA(s) which are characterized by tissue-specific, cell type-specific, cell state-specific or species-specific expression.

As described herein, a tissue-specific, cell type-specific, cell state-specific or species-specific MSE useful in the RNA-based circuits of the present disclosure can be derived from those MSEs that bind, and are post-transcriptionally inhibited by, miRNAs which are expressed at high levels in a particular tissue or cell type, or at a particular cell state, or in a select species, but are not expressed or expressed at very low levels in other tissue or cell types, or other cell states, or in other species. However, in some cases, MSEs that bind, and are post-transcriptionally inhibited by, miRNAs which are expressed at low levels in a particular tissue or cell type, or at a particular cell state, or in a select species may also be useful, for example, for achieving a desired level of expression of a particular payload polypeptide.

miRNAs that are “highly expressed” or “expressed at high levels” can be detected by standard Northern blot analysis (Pall et al., Nature Protocols 3 (6) 1077 (2008)). Preferably, such highly expressed miRNAs represent greater than or equal to 0.1% of the total cellular miRNA found in the tissue or cell of interest as measured by RNA deep sequencing (Hafner et al., Methods 44 (1) 3 (2008)).

miRNAs that are “expressed at very low levels” may be undetectable by standard Northern blot analysis. Preferably, such miRNAs expressed at very low levels represent equal to or less than 0.01% of the total cellular miRNA found in the tissue or cell of interest as measured by RNA deep sequencing.

In some embodiments, the one or more MSE sequences is targeted by miR-1, miR-155, miR-124, miR-182, miR-96, miR-183, miR-21, miR-16, miR-17, miR-19, miR-25, miR-34, miR-92, miR-93, miR-142, miR-222, miR-149, miR-181b-2, or a combination thereof.

In some embodiments, the one or more MSE sequences is targeted by aae-bantam-5p, aae-let-7, aae-miR-1, aae-miR-10, aae-miR-100, aae-miR-1000, aae-miR-10365, aae-miR-11-3p, aae-miR-11-5p, aae-miR-1174, aae-miR-1175-3p, aae-miR-1175-5p, aae-miR-11893, aae-miR-11894a, aae-miR-11894b, aae-miR-11895, aae-miR-11896, aae-miR-11897a, aae-miR-11897b, aae-miR-11898, aae-miR-11899, aae-miR-11900, aae-miR-11901, aae-miR-11902, aae-miR-11903a, aae-miR-11903b, aae-miR-11904, aae-miR-11905, aae-miR-11906, aae-miR-11907, aae-miR-11908, aae-miR-11909, aae-miR-11910, aae-miR-11911, aae-miR-11912, aae-miR-11913, aae-miR-11914, aae-miR-11915, aae-miR-11916, aae-miR-11917, aae-miR-11918, aae-miR-11919, aae-miR-11920, aae-miR-11921, aae-miR-11922, aae-miR-11923, aae-miR-11924, aae-miR-11925, aae-miR-11926, aae-miR-11927, aae-miR-11928, aae-miR-12-3p, aae-miR-12-5p, aae-miR-124, aae-miR-125-5p, aae-miR-13-3p, aae-miR-13-5p, aae-miR-133, aae-miR-137, aae-miR-14, aae-miR-184, aae-miR-1889-3p, aae-miR-1889-5p, aae-miR-1890, aae-miR-1891, aae-miR-190, aae-miR-193, aae-miR-210, aae-miR-219, aae-miR-252-3p, aae-miR-252-5p, aae-miR-263a-3p, aae-miR-263a-5p, aae-miR-263b-3p, aae-miR-263b-5p, aae-miR-275-3p, aae-miR-275-5p, aae-miR-276-3p, aae-miR-276-5p, aae-miR-2765, aae-miR-277-3p, aae-miR-277-5p, aae-miR-278-3p, aae-miR-278-5p, aae-miR-279, aae-miR-281-3p, aae-miR-281-5p, aae-miR-282-3p, aae-miR-282-5p, aae-miR-283, aae-miR-285, aae-miR-286a, aae-miR-286b, aae-miR-2940-3p, aae-miR-2940-5p, aae-miR-2941, aae-miR-2942, aae-miR-2943, aae-miR-2944a-3p, aae-miR-2944a-5p, aae-miR-2944b-3p, aae-miR-2944b-5p, aae-miR-2945-3p, aae-miR-2945-5p, aae-miR-2946, aae-miR-2a-3p, aae-miR-2a-5p, aae-miR-2b, aae-miR-2c, aae-miR-305-3p, aae-miR-305-5p, aae-miR-306-3p, aae-miR-306-5p, aae-miR-307, aae-miR-308-3p, aae-miR-308-5p, aae-miR-309a, aae-miR-309b-3p, aae-miR-309b-5p, aae-miR-31, aae-miR-315-3p, aae-miR-315-5p, aae-miR-316, aae-miR-317, aae-miR-33, aae-miR-34-3p, aae-miR-34-5p, aae-miR-375, aae-miR-7, aae-miR-71-3p, aae-miR-71-5p, aae-miR-79-3p, aae-miR-79-5p, aae-miR-8-3p, aae-miR-8-5p, aae-miR-87, aae-miR-927, aae-miR-929, aae-miR-92a-3p, aae-miR-92a-5p, aae-miR-92b-3p, aae-miR-92b-5p, aae-miR-932-3p, aae-miR-932-5p, aae-miR-957, aae-miR-965, aae-miR-970, aae-miR-980-3p, aae-miR-980-5p, aae-miR-981, aae-miR-988-3p, aae-miR-988-5p, aae-miR-989, aae-miR-993, aae-miR-996, aae-miR-998, aae-miR-999, aae-miR-9a, aae-miR-9b, aae-miR-9c-3p, aae-miR-9c-5p, aae-miR-iab-4-3p, aae-miR-iab-4-5p, aau-miR160, aau-miR162, aau-miR168, aau-miR172, aau-miR2086, aau-miR319, aau-miR396, abu-let-7a, abu-let-7b, abu-let-7c, abu-let-7d, abu-let-7e, abu-let-7f, abu-let-7g, abu-let-7h, abu-let-7i, abu-miR-1, abu-miR-100, abu-miR-101a, abu-miR-101b, abu-miR-103, abu-miR-10543, abu-miR-10544, abu-miR-10545-3p, abu-miR-10545-5p, abu-miR-10546, abu-miR-10547, abu-miR-10548-3p, abu-miR-10548-5p, abu-miR-10549, abu-miR-10550-3p, abu-miR-10550-5p, abu-miR-10551, abu-miR-10552, abu-miR-10553, abu-miR-10554, abu-miR-10555, abu-miR-10556a, abu-miR-10556b, abu-miR-10557, abu-miR-10558, abu-miR-10559, abu-miR-10560, abu-miR-10561, abu-miR-10562, abu-miR-10563, abu-miR-10564, abu-miR-10565, abu-miR-106, abu-miR-107, abu-miR-10a, abu-miR-10b, abu-miR-10c, abu-miR-10d, abu-miR-122, abu-miR-124, abu-miR-125a, abu-miR-125b, abu-miR-126-3p, abu-miR-126-5p, abu-miR-128, abu-miR-129, abu-miR-130, abu-miR-132-3p, abu-miR-132-5p, abu-miR-133a, abu-miR-133b, abu-miR-135a-3p, abu-miR-135a-5p, abu-miR-135b, abu-miR-135c-3p, abu-miR-135c-5p, abu-miR-135d, abu-miR-137, abu-miR-138, abu-miR-1388, abu-miR-139, abu-miR-140, abu-miR-142-3p, abu-miR-142-5p, abu-miR-143, abu-miR-144, abu-miR-145, abu-miR-146, abu-miR-147, abu-miR-148a-3p, abu-miR-148a-5p, abu-miR-148b, abu-miR-150, abu-miR-152, abu-miR-153a, abu-miR-153b, abu-miR-153c, abu-miR-155, abu-miR-15a, abu-miR-15b, abu-miR-15c-3p, abu-miR-15c-5p, abu-miR-16a, abu-miR-16b, abu-miR-16c, abu-miR-17, abu-miR-1788, abu-miR-181a, abu-miR-181b, abu-miR-181c, abu-miR-182, abu-miR-183, abu-miR-184a, abu-miR-184b, abu-miR-187, abu-miR-18a, abu-miR-18b, abu-miR-190a-3p, abu-miR-190a-5p, abu-miR-190b, abu-miR-192, abu-miR-193-3p, abu-miR-193-5p, abu-miR-194a, abu-miR-194b, abu-miR-196a, abu-miR-196b, abu-miR-199-3p, abu-miR-199-5p, abu-miR-19a, abu-miR-19b, abu-miR-19c, abu-miR-19d, abu-miR-200a, abu-miR-200b, abu-miR-202, abu-miR-203, abu-miR-204, abu-miR-205, abu-miR-206, abu-miR-20a, abu-miR-20b, abu-miR-21, abu-miR-210, abu-miR-212-3p, abu-miR-214, abu-miR-216a, abu-miR-216b, abu-miR-2187a, abu-miR-2187b-3p, abu-miR-2187b-5p, abu-miR-2188, abu-miR-218a, abu-miR-218b, abu-miR-219-5p, abu-miR-221, abu-miR-222, abu-miR-22a, abu-miR-22b, abu-miR-22c, abu-miR-23a, abu-miR-23b, abu-miR-23c, abu-miR-24a, abu-miR-24b-3p, abu-miR-24b-5p, abu-miR-24c, abu-miR-25, abu-miR-26a, abu-miR-26b, abu-miR-27a, abu-miR-27b, abu-miR-27c, abu-miR-27d-3p, abu-miR-27d-5p, abu-miR-27e, abu-miR-29a, abu-miR-29b, abu-miR-29c-3p, abu-miR-29c-5p, abu-miR-29d, abu-miR-301a, abu-miR-301b, abu-miR-30a-3p, abu-miR-30a-5p, abu-miR-30b, abu-miR-30c, abu-miR-30d, abu-miR-31, abu-miR-3120, abu-miR-33-5p, abu-miR-338, abu-miR-34, abu-miR-365, abu-miR-375, abu-miR-429b, abu-miR-430, abu-miR-449a, abu-miR-449b-3p, abu-miR-449b-5p, abu-miR-451, abu-miR-454b, abu-miR-455, abu-miR-456, abu-miR-457, abu-miR-458, abu-miR-460, abu-miR-462, abu-miR-489, abu-miR-499, abu-miR-551, abu-miR-7, abu-miR-7132a-3p, abu-miR-7132a-5p, abu-miR-7132b-3p, abu-miR-7132b-5p, abu-miR-7133-3p, abu-miR-7133-5p, abu-miR-7147, abu-miR-722, abu-miR-723a, abu-miR-723b, abu-miR-724, abu-miR-725, abu-miR-726, abu-miR-727a-5p, abu-miR-727b-3p, abu-miR-728a, abu-miR-728b, abu-miR-729, abu-miR-730, abu-miR-731-3p, abu-miR-731-5p, abu-miR-734, abu-miR-736, abu-miR-737, abu-miR-7552, abu-miR-8160a, abu-miR-8160b, abu-miR-9-3p, abu-miR-9-5p, abu-miR-92a, abu-miR-92b, abu-miR-93, abu-miR-96, abu-miR-99a, abu-miR-99b, aca-let-7a-2-3p, aca-let-7a-3-3p, aca-let-7a-3p, aca-let-7a-5p, aca-let-7b-3p, aca-let-7b-5p, aca-let-7c-1-3p, aca-let-7c-2-3p, aca-let-7c-3-3p, aca-let-7c-5p, aca-let-7d-3p, aca-let-7d-5p, aca-let-7e-3p, aca-let-7e-5p, aca-let-7f-1-3p, aca-let-7f-2-3p, aca-let-7f-5p, aca-let-7g, aca-let-7i-3p, aca-let-7i-5p, aca-miR-100, aca-miR-101-1-5p, aca-miR-101-2-5p, aca-miR-101-3p, aca-miR-103-3p, aca-miR-103-5p, aca-miR-106-3p, aca-miR-106-5p, aca-miR-107-3p, aca-miR-107-5p, aca-miR-10a-3p, aca-miR-10a-5p, aca-miR-10b-3p, aca-miR-10b-5p, aca-miR-10c-3p, aca-miR-10c-5p, aca-miR-122-3p, aca-miR-122-5p, aca-miR-124a, aca-miR-124b, aca-miR-125a-3p, aca-miR-125a-5p, aca-miR-125b, aca-miR-126-3p, aca-miR-126-5p, aca-miR-128-3p, aca-miR-128-5p, aca-miR-129a-3p, aca-miR-129a-5p, aca-miR-129b-3p, aca-miR-129b-5p, aca-miR-1306, aca-miR-130a-1-5p, aca-miR-130a-2-5p, aca-miR-130a-3p, aca-miR-130b-3p, aca-miR-130b-5p, aca-miR-130c, aca-miR-132-3p, aca-miR-132-5p, aca-miR-1329-3p, aca-miR-1329-5p, aca-miR-133a, aca-miR-133a-3p, aca-miR-133b-3p, aca-miR-133b-5p, aca-miR-135-1-3p, aca-miR-135-2-3p, aca-miR-135-3-3p, aca-miR-135-5p, aca-miR-137a, aca-miR-137b-3p, aca-miR-137b-5p, aca-miR-138-1-3p, aca-miR-138-2-3p, aca-miR-138-5p, aca-miR-1388-3p, aca-miR-1388-5p, aca-miR-139-3p, aca-miR-139-5p, aca-miR-1397, aca-miR-140-3p, aca-miR-140-5p, aca-miR-142-3p, aca-miR-142-5p, aca-miR-143-3p, aca-miR-143-5p, aca-miR-144-3p, aca-miR-144-5p, aca-miR-145-3p, aca-miR-145-5p, aca-miR-146a-3p, aca-miR-146a-5p, aca-miR-146b-3p, aca-miR-146b-5p, aca-miR-147, aca-miR-148a-3p, aca-miR-148a-5p, aca-miR-148b-3p, aca-miR-148b-5p, aca-miR-150-3p, aca-miR-150-5p, aca-miR-153-3p, aca-miR-153-5p, aca-miR-155-3p, aca-miR-155-5p, aca-miR-15a-3p, aca-miR-15a-5p, aca-miR-15b-3p, aca-miR-15b-5p, aca-miR-1641-5p, aca-miR-1662-3p, aca-miR-1662-5p, aca-miR-1677a, aca-miR-1677b-3p, aca-miR-1677b-5p, aca-miR-16a-3p, aca-miR-16a-5p, aca-miR-16b-3p, aca-miR-16b-5p, aca-miR-17-3p, aca-miR-17-5p, aca-miR-1788-3p, aca-miR-1788-5p, aca-miR-1805-3p, aca-miR-1805-5p, aca-miR-181a, aca-miR-181b, aca-miR-181c-5p, aca-miR-182-3p, aca-miR-182-5p, aca-miR-184-3p, aca-miR-184-5p, aca-miR-187-3p, aca-miR-187-5p, aca-miR-18a-3p, aca-miR-18a-5p, aca-miR-18b-3p, aca-miR-18b-5p, aca-miR-190a-3p, aca-miR-190a-5p, aca-miR-190b, aca-miR-191-3p, aca-miR-191-5p, aca-miR-193-3p, aca-miR-193-5p, aca-miR-194-1-3p, aca-miR-194-2-3p, aca-miR-194-5p, aca-miR-196a, aca-miR-196c-3p, aca-miR-196c-5p, aca-miR-199a-5p, aca-miR-199b-5p, aca-miR-19a-3p, aca-miR-19a-5p, aca-miR-19b, aca-miR-19c-3p, aca-miR-1a-1-5p, aca-miR-1a-2-5p, aca-miR-1a-3p, aca-miR-1b-3p, aca-miR-1b-5p, aca-miR-200a-3p, aca-miR-200a-5p, aca-miR-200b-3p, aca-miR-200b-5p, aca-miR-202-3p.1, aca-miR-202-5p, aca-miR-202-5p.2, aca-miR-203-3p, aca-miR-203-5p, aca-miR-204a-3p, aca-miR-204a-5p, aca-miR-205a, aca-miR-205b, aca-miR-206-3p, aca-miR-206-5p, aca-miR-208-3p, aca-miR-208-5p, aca-miR-20a-3p, aca-miR-20a-5p, aca-miR-20b-3p, aca-miR-20b-5p, aca-miR-21-3p, aca-miR-21-5p, aca-miR-210-3p, aca-miR-210-5p, aca-miR-212-3p, aca-miR-212-5p, aca-miR-214-3p, aca-miR-214-5p, aca-miR-215-3p, aca-miR-215-5p, aca-miR-216a, aca-miR-216b-3p, aca-miR-216b-5p, aca-miR-217-3p, aca-miR-217-5p, aca-miR-218-3p, aca-miR-218-5p, aca-miR-2188-3p, aca-miR-2188-5p, aca-miR-219-1-3p, aca-miR-219-2-3p, aca-miR-219-5p, aca-miR-22-3p, aca-miR-22-5p, aca-miR-221-3p, aca-miR-221-5p, aca-miR-222a-3p, aca-miR-222a-5p, aca-miR-222b-3p, aca-miR-222b-5p, aca-miR-223-3p, aca-miR-223-5p, aca-miR-23a-3p, aca-miR-23a-5p, aca-miR-23b-3p, aca-miR-23b-5p, aca-miR-24-1-5p, aca-miR-24-2-5p, aca-miR-24-3p, aca-miR-26-1-3p, aca-miR-26-2-3p, aca-miR-26-3-5p, aca-miR-26-3p, aca-miR-26-5p, aca-miR-27a-3p, aca-miR-27a-5p, aca-miR-27b-3p, aca-miR-27b-5p, aca-miR-2970-3p, aca-miR-2970-5p, aca-miR-29a-1-5p, aca-miR-29a-2-5p, aca-miR-29a-3p, aca-miR-29b, aca-miR-301a-3p, aca-miR-301a-5p, aca-miR-301b-3p, aca-miR-301b-5p, aca-miR-302a-3p, aca-miR-302b, aca-miR-30a-3p, aca-miR-30a-5p, aca-miR-30b-3p, aca-miR-30b-5p, aca-miR-30c-2-3p, aca-miR-30c-3p, aca-miR-30c-5p, aca-miR-30d-3p, aca-miR-30d-5p, aca-miR-30e-3p, aca-miR-30e-5p, aca-miR-31-3p, aca-miR-31-5p, aca-miR-32-3p, aca-miR-32-5p, aca-miR-33-1-3p, aca-miR-33-2-3p, aca-miR-33-5p, aca-miR-338-3p, aca-miR-338-5p, aca-miR-34a-3p, aca-miR-34a-5p, aca-miR-34b-3p, aca-miR-34b-5p, aca-miR-34c-3p, aca-miR-34c-5p, aca-miR-363-3p, aca-miR-363-5p, aca-miR-365-3p, aca-miR-365-5p, aca-miR-367, aca-miR-375-3p, aca-miR-375-5p, aca-miR-383-3p, aca-miR-383-5p, aca-miR-425-3p, aca-miR-425-5p, aca-miR-429-3p, aca-miR-429-5p, aca-miR-449a, aca-miR-449b, aca-miR-449c-3p, aca-miR-449c-5p, aca-miR-451-3p, aca-miR-451-5p, aca-miR-454-3p, aca-miR-454-5p, aca-miR-455-3p, aca-miR-455-5p, aca-miR-456, aca-miR-457-3p, aca-miR-457-5p, aca-miR-458, aca-miR-460a-3p, aca-miR-460a-5p, aca-miR-460b-3p, aca-miR-460b-5p, aca-miR-489-3p, aca-miR-489-5p, aca-miR-490-3p, aca-miR-490-5p, aca-miR-497-3p, aca-miR-497-5p, aca-miR-499-3p, aca-miR-499-5p, aca-miR-5390, aca-miR-5391, aca-miR-5392, aca-miR-5393, aca-miR-5394-3p, aca-miR-5394-5p, aca-miR-5395, aca-miR-5396a, aca-miR-5396b-3p, aca-miR-5396b-5p, aca-miR-5397-3p, aca-miR-5397-5p, aca-miR-5398-3p, aca-miR-5398-5p, aca-miR-5399-3p, aca-miR-5399-5p, aca-miR-5400, aca-miR-5401, aca-miR-5402-3p, aca-miR-5402-5p, aca-miR-5403, aca-miR-5404, aca-miR-5405-3p, aca-miR-5405-5p, aca-miR-5406-3p, aca-miR-5406-5p, aca-miR-5407-3p, aca-miR-5407-5p, aca-miR-5408a-3p, aca-miR-5408a-5p, aca-miR-5408b, aca-miR-5408c-3p, aca-miR-5408c-5p, aca-miR-5409, aca-miR-5410-3p, aca-miR-5410-5p, aca-miR-5411, aca-miR-5412-3p, aca-miR-5412-5p, aca-miR-5413-3p, aca-miR-5413-5p, aca-miR-5414, aca-miR-5415, aca-miR-5416, aca-miR-5417, aca-miR-5418, aca-miR-5419, aca-miR-5420, aca-miR-5421, aca-miR-5422, aca-miR-5423, aca-miR-5424, aca-miR-5425, aca-miR-5426-3p, aca-miR-5426-5p, aca-miR-5427, aca-miR-5428, aca-miR-5429, aca-miR-5430, aca-miR-5431, aca-miR-5432, aca-miR-5433, aca-miR-5434, aca-miR-5435a, aca-miR-5435b-3p, aca-miR-5435b-5p, aca-miR-5435c-3p, aca-miR-5435c-5p, aca-miR-5436-3p, aca-miR-5436-5p, aca-miR-5437, aca-miR-5438, aca-miR-5439, aca-miR-5440, aca-miR-5441, aca-miR-5442, aca-miR-5443, aca-miR-5444a, aca-miR-5444b, aca-miR-5445, aca-miR-5446, aca-miR-5447, aca-miR-5448, aca-miR-5449, aca-miR-5450, aca-miR-5451, aca-miR-5452-3p, aca-miR-5452-5p, aca-miR-5453, aca-miR-5454, aca-miR-5455-3p, aca-miR-5455-5p, aca-miR-5456, aca-miR-5457, aca-miR-5458, aca-miR-5459, aca-miR-5460, aca-miR-5461, aca-miR-5462, aca-miR-5463, aca-miR-5464-3p, aca-miR-5464-5p, aca-miR-5465, aca-miR-5466, aca-miR-5467, aca-miR-5468, aca-miR-5469, aca-miR-5470, aca-miR-551, aca-miR-7-1-3p, aca-miR-7-2-3p, aca-miR-7-5p, aca-miR-726, aca-miR-727-3p, aca-miR-727-5p, aca-miR-737-3p, aca-miR-737-5p, aca-miR-875, aca-miR-9-1-3p, aca-miR-9-3-3p, aca-miR-9-5p, aca-miR-92a, aca-miR-9572-3p, aca-miR-9572-5p, aca-miR-9573-3p, aca-miR-9573-5p, aca-miR-9574-3p, aca-miR-9574-5p, aca-miR-9575-3p, aca-miR-9575-5p, aca-miR-9576-3p, aca-miR-9576-5p, aca-miR-9577-3p, aca-miR-9577-5p, aca-miR-98-3p, aca-miR-98-5p, aca-miR-99a-3p, aca-miR-99a-5p, aca-miR-99b-3p, aca-miR-99b-5p, aga-bantam, aga-let-7, aga-miR-1, aga-miR-10, aga-miR-100, aga-miR-1000, aga-miR-10355-3p, aga-miR-10355-5p, aga-miR-10356-3p, aga-miR-10356-5p, aga-miR-10357-3p, aga-miR-10357-5p, aga-miR-10358-3p, aga-miR-10358-5p, aga-miR-10359-3p, aga-miR-10359-5p, aga-miR-10360-3p, aga-miR-10360-5p, aga-miR-10361a-3p, aga-miR-10361a-5p, aga-miR-10361b-3p, aga-miR-10361b-5p, aga-miR-10362-3p, aga-miR-10362-5p, aga-miR-10363-3p, aga-miR-10363-5p, aga-miR-10364-3p, aga-miR-10364-5p, aga-miR-10365-3p, aga-miR-10365-5p, aga-miR-10366a-2-5p, aga-miR-10366a-3p, aga-miR-10366a-5p, aga-miR-10366b-3p, aga-miR-10366b-5p, aga-miR-10367-3p, aga-miR-10367-5p, aga-miR-10368-3p, aga-miR-10368-5p, aga-miR-10369-3p, aga-miR-10369-5p, aga-miR-10370-2-5p, aga-miR-10370-3p, aga-miR-10370-5p, aga-miR-10371-3p, aga-miR-10371-5p, aga-miR-10372a-3p, aga-miR-10372a-5p, aga-miR-10372b-3p, aga-miR-10372b-5p, aga-miR-10373-3p, aga-miR-10373-5p, aga-miR-10374-3p, aga-miR-10375a-3p, aga-miR-10375a-5p, aga-miR-10375b-3p, aga-miR-10375b-5p, aga-miR-10376-3p, aga-miR-10376-5p, aga-miR-10377a-3p, aga-miR-10377a-5p, aga-miR-10377b-3p, aga-miR-10377b-5p, aga-miR-10378-3p, aga-miR-10378-5p, aga-miR-10379-3p, aga-miR-10379-5p, aga-miR-10380-3p, aga-miR-10380-5p, aga-miR-10381-5p, aga-miR-11, aga-miR-1174, aga-miR-1175, aga-miR-12, aga-miR-124, aga-miR-12413-3p, aga-miR-12414-3p, aga-miR-12415-3p, aga-miR-12416-3p, aga-miR-12417-5p, aga-miR-12418-3p, aga-miR-12419-5p, aga-miR-125, aga-miR-133, aga-miR-137, aga-miR-13b, aga-miR-14, aga-miR-184, aga-miR-1889, aga-miR-1890, aga-miR-1891, aga-miR-190, aga-miR-2, aga-miR-210, aga-miR-219, aga-miR-263a, aga-miR-263b, aga-miR-275, aga-miR-276-3p, aga-miR-276-5p, aga-miR-277, aga-miR-278, aga-miR-279, aga-miR-2796-3p, aga-miR-2796-5p, aga-miR-281, aga-miR-282, aga-miR-283, aga-miR-285-3p, aga-miR-285-5p, aga-miR-286, aga-miR-286b-3p, aga-miR-286b-5p, aga-miR-2944a-3p, aga-miR-2944a-5p, aga-miR-2944b-3p, aga-miR-2944b-5p, aga-miR-2945-3p, aga-miR-2945-5p, aga-miR-2b-3p, aga-miR-2b-5p, aga-miR-2c-3p, aga-miR-2c-5p, aga-miR-305, aga-miR-306, aga-miR-307, aga-miR-308, aga-miR-309, aga-miR-31-3p, aga-miR-31-5p, aga-miR-315, aga-miR-317, aga-miR-33-3p, aga-miR-33-5p, aga-miR-34, aga-miR-375, aga-miR-7, aga-miR-79, aga-miR-8, aga-miR-87, aga-miR-927, aga-miR-929, aga-miR-92a, aga-miR-92b, aga-miR-932-3p, aga-miR-932-5p, aga-miR-957, aga-miR-965-3p, aga-miR-965-5p, aga-miR-970, aga-miR-980-3p, aga-miR-980-5p, aga-miR-981, aga-miR-988, aga-miR-989, aga-miR-993, aga-miR-996, aga-miR-998-3p, aga-miR-998-5p, aga-miR-9a, aga-miR-9b, aga-miR-9c, aga-miR-iab-4, age-miR-100, age-miR-101, age-miR-103, age-miR-106a, age-miR-106b, age-miR-10a, age-miR-124a, age-miR-125b, age-miR-127, age-miR-128, age-miR-133a, age-miR-135, age-miR-15a, age-miR-15b, age-miR-16, age-miR-17-3p, age-miR-17-5p, age-miR-18, age-miR-194, age-miR-196, age-miR-197, age-miR-198, age-miR-19a, age-miR-19b, age-miR-20, age-miR-205, age-miR-21, age-miR-214, age-miR-218, age-miR-22, age-miR-222, age-miR-23a, age-miR-27a, age-miR-28, age-miR-29a, age-miR-29b, age-miR-30b, age-miR-34a, age-miR-506, age-miR-507, age-miR-508, age-miR-509a, age-miR-509b, age-miR-510, age-miR-513a, age-miR-513b, age-miR-513c, age-miR-513d, age-miR-513e, age-miR-514, age-miR-9, age-miR-92, age-miR-93, age-miR-98, ahy-miR156a, ahy-miR156b-3p, ahy-miR156b-5p, ahy-miR156c, ahy-miR159, ahy-miR160-3p, ahy-miR160-5p, ahy-miR167-3p, ahy-miR167-5p, ahy-miR3508, ahy-miR3509-3p, ahy-miR3509-5p, ahy-miR3510, ahy-miR3511-3p, ahy-miR3511-5p, ahy-miR3512, ahy-miR3513-3p, ahy-miR3513-5p, ahy-miR3514-3p, ahy-miR3514-5p, ahy-miR3515, ahy-miR3516, ahy-miR3517, ahy-miR3518, ahy-miR3519, ahy-miR3520-3p, ahy-miR3520-5p, ahy-miR3521, ahy-miR394, ahy-miR398, ahy-miR408-3p, ahy-miR408-5p, aja-let-7i, aja-miR-142, aja-miR-143, aja-miR-144, aja-miR-145, aja-miR-1893, aja-miR-21, aja-miR-214, aja-miR-22, aja-miR-25, aja-miR-29b, aja-miR-29c, aja-miR-3074, aja-miR-3120, aja-miR-331, aja-miR-3596, aja-miR-3618, aja-miR-671, aja-miR-7, aly-miR156a-3p, aly-miR156a-5p, aly-miR156b-3p, aly-miR156b-5p, aly-miR156c-3p, aly-miR156c-5p, aly-miR156d-3p, aly-miR156d-5p, aly-miR156e-3p, aly-miR156e-5p, aly-miR156f-3p, aly-miR156f-5p, aly-miR156g-3p, aly-miR156g-5p, aly-miR156 h-3p, aly-miR156 h-5p, aly-miR157a-3p, aly-miR157a-5p, aly-miR157b-3p, aly-miR157b-5p, aly-miR157c-3p, aly-miR157c-5p, aly-miR157d-3p, aly-miR157d-5p, aly-miR158a-3p, aly-miR158a-5p, aly-miR158b-3p, aly-miR158b-5p, aly-miR159a-3p, aly-miR159a-5p, aly-miR159b-3p, aly-miR159b-5p, aly-miR159c-3p, aly-miR159c-5p, aly-miR160a-3p, aly-miR160a-5p, aly-miR160b-3p, aly-miR160b-5p, aly-miR160c-3p, aly-miR160c-5p, aly-miR161-3p.1, aly-miR161-3p.2, aly-miR161-5p.1, aly-miR161-5p.2, aly-miR162a-3p, aly-miR162a-5p, aly-miR162b-3p, aly-miR162b-5p, aly-miR163-3p.1, aly-miR163-3p.2, aly-miR163-5p.1, aly-miR163-5p.2, aly-miR164a-3p, aly-miR164a-5p, aly-miR164b-3p, aly-miR164b-5p, aly-miR164c-3p, aly-miR164c-5p, aly-miR165a-3p, aly-miR165a-5p, aly-miR165b-3p, aly-miR165b-5p, aly-miR166a-3p, aly-miR166a-5p, aly-miR166b-3p, aly-miR166b-5p, aly-miR166c-3p, aly-miR166c-5p, aly-miR166d-3p, aly-miR166d-5p, aly-miR166e-3p, aly-miR166e-5p, aly-miR166f-3p, aly-miR166f-5p, aly-miR166g-3p, aly-miR166g-5p, aly-miR166 h-3p, aly-miR166 h-5p, aly-miR167a-3p, aly-miR167a-5p, aly-miR167b-3p, aly-miR167b-5p, aly-miR167c-3p, aly-miR167c-5p, aly-miR167d-3p, aly-miR167d-5p, aly-miR168a-3p, aly-miR168a-5p, aly-miR168b-3p, aly-miR168b-5p, aly-miR169a-3p, aly-miR169a-5p, aly-miR169b-3p, aly-miR169b-5p, aly-miR169c-3p, aly-miR169c-5p, aly-miR169d-3p, aly-miR169d-5p, aly-miR169e-3p, aly-miR169e-5p, aly-miR169f-3p, aly-miR169f-5p, aly-miR169g-3p, aly-miR169g-5p, aly-miR169 h-3p, aly-miR169 h-5p, aly-miR169i-3p, aly-miR169i-5p, aly-miR169j-3p, aly-miR169j-5p, aly-miR169k-3p, aly-miR169k-5p, aly-miR1691-3p, aly-miR1691-5p, aly-miR169m-3p, aly-miR169m-5p, aly-miR169n-3p, aly-miR169n-5p, aly-miR170-3p, aly-miR170-5p, aly-miR171a-3p, aly-miR171a-5p, aly-miR171b-3p, aly-miR171b-5p, aly-miR171c-3p, aly-miR171c-5p, aly-miR172a-3p, aly-miR172a-5p, aly-miR172b-3p, aly-miR172b-5p, aly-miR172c-3p, aly-miR172c-5p, aly-miR172d-3p, aly-miR172d-5p, aly-miR172e-3p, aly-miR172e-5p, aly-miR172f-3p, aly-miR172f-5p, aly-miR173a-3p, aly-miR173a-5p, aly-miR173b-3p, aly-miR173b-5p, aly-miR1887, aly-miR2111a-3p, aly-miR2111a-5p, aly-miR2111b-3p, aly-miR2111b-5p, aly-miR2111c-3p, aly-miR2111c-5p, aly-miR2112-3p, aly-miR2112-5p, aly-miR319a-3p, aly-miR319a-5p, aly-miR319b-3p, aly-miR319b-5p, aly-miR319c-3p, aly-miR319c-5p, aly-miR319d-3p, aly-miR319d-5p, aly-miR3433-3p, aly-miR3433-5p, aly-miR3434-3p, aly-miR3434-5p, aly-miR3435-3p, aly-miR3435-5p, aly-miR3436-3p, aly-miR3436-5p, aly-miR3437-3p, aly-miR3437-5p, aly-miR3438-3p, aly-miR3438-5p, aly-miR3439-3p, aly-miR3439-5p, aly-miR3440-3p, aly-miR3440-5p, aly-miR3441-3p.1, aly-miR3441-3p.2, aly-miR3441-5p.1, aly-miR3441-5p.2, aly-miR3442-3p, aly-miR3442-5p, aly-miR3443-3p, aly-miR3443-5p, aly-miR3444a-3p, aly-miR3444a-5p, aly-miR3444b, aly-miR3445-3p.1, aly-miR3445-3p.2, aly-miR3445-5p.1, aly-miR3445-5p.2, aly-miR3446-3p, aly-miR3446-5p, aly-miR3447-3p, aly-miR3447-5p, aly-miR3448-3p, aly-miR3448-5p, aly-miR3449-3p, aly-miR3449-5p, aly-miR390a-3p, aly-miR390a-5p, aly-miR390b-3p, aly-miR390b-5p, aly-miR391-3p, aly-miR391-5p, aly-miR393a-3p, aly-miR393a-5p, aly-miR393b-3p, aly-miR393b-5p, aly-miR394a-3p, aly-miR394a-5p, aly-miR394b-3p, aly-miR394b-5p, aly-miR395b-3p, aly-miR395b-5p, aly-miR395c-3p, aly-miR395c-5p, aly-miR395d-3p, aly-miR395d-5p, aly-miR395e-3p, aly-miR395e-5p, aly-miR395f-3p, aly-miR395f-5p, aly-miR395g-3p, aly-miR395g-5p, aly-miR395 h-3p, aly-miR395 h-5p, aly-miR395i, aly-miR396a-3p, aly-miR396a-5p, aly-miR396b-3p, aly-miR396b-5p, aly-miR397a-3p, aly-miR397a-5p, aly-miR397b-3p, aly-miR397b-5p, aly-miR398a-3p, aly-miR398a-5p, aly-miR398b-3p, aly-miR398b-5p, aly-miR398c-3p, aly-miR398c-5p, aly-miR399a-3p, aly-miR399a-5p, aly-miR399b-3p, aly-miR399b-5p, aly-miR399c-3p, aly-miR399c-5p, aly-miR399d-3p, aly-miR399d-5p, aly-miR399e-3p, aly-miR399e-5p, aly-miR399f-3p, aly-miR399f-5p, aly-miR399g-3p, aly-miR399g-5p, aly-miR399 h-3p, aly-miR399 h-5p, aly-miR399i-3p, aly-miR399i-5p, aly-miR400-3p, aly-miR400-5p, aly-miR402-3p, aly-miR402-5p, aly-miR403a-3p, aly-miR403a-5p, aly-miR403b-3p, aly-miR403b-5p, aly-miR408-3p, aly-miR408-5p, aly-miR4221, aly-miR4222, aly-miR4223, aly-miR4224, aly-miR4225, aly-miR4226, aly-miR4227, aly-miR4228, aly-miR4229, aly-miR4230, aly-miR4231, aly-miR4232, aly-miR4233, aly-miR4234, aly-miR4235, aly-miR4236, aly-miR4237, aly-miR4238, aly-miR4239, aly-miR4240, aly-miR4241, aly-miR4242, aly-miR4243, aly-miR4244, aly-miR4245, aly-miR4246, aly-miR4247, aly-miR4248a, aly-miR4248b, aly-miR4248c, aly-miR4249, aly-miR4250, aly-miR472-3p, aly-miR472-5p, aly-miR771-3p, aly-miR771-5p, aly-miR773b-3p, aly-miR773b-5p, aly-miR774a-3p.1, aly-miR774a-3p.2, aly-miR774a-5p.1, aly-miR774a-5p.2, aly-miR774b-3p, aly-miR774b-5p, aly-miR781-3p, aly-miR781-5p, aly-miR822-3p, aly-miR822-5p, aly-miR823-3p, aly-miR823-5p, aly-miR824-3p, aly-miR824-5p, aly-miR825-3p, aly-miR825-5p, aly-miR827-3p, aly-miR827-5p, aly-miR828-3p, aly-miR828-5p, aly-miR829-3p, aly-miR829-5p, aly-miR831-3p, aly-miR831-5p, aly-miR833-3p, aly-miR833-5p, aly-miR834-3p, aly-miR834-5p, aly-miR835-3p, aly-miR835-5p, aly-miR837-3p, aly-miR837-5p, aly-miR838-3p, aly-miR838-5p, aly-miR839-3p, aly-miR839-5p, aly-miR840-3p, aly-miR840-5p, aly-miR841, aly-miR842-3p, aly-miR842-5p, aly-miR844-3p, aly-miR844-5p, aly-miR845a-3p, aly-miR845a-5p, aly-miR845b-3p, aly-miR845b-5p, aly-miR846-3p, aly-miR846-5p, aly-miR847-3p, aly-miR847-5p, aly-miR848-3p, aly-miR848-5p, aly-miR851-3p, aly-miR851-5p, aly-miR852-3p, aly-miR852-5p, aly-miR853-3p, aly-miR853-5p, aly-miR856-3p, aly-miR856-5p, aly-miR857-3p, aly-miR857-5p, aly-miR858-3p, aly-miR858-5p, aly-miR859-3p, aly-miR859-5p, aly-miR860-3p, aly-miR860-5p, aly-miR861-3p, aly-miR861-5p, aly-miR862-3p, aly-miR862-5p, aly-miR868-3p, aly-miR868-5p, aly-miR869-3p, aly-miR869-5p, ama-miR156, ama-miR396-3p, ama-miR396-5p, ame-bantam-3p, ame-let-7-5p, ame-miR-1-3p, ame-miR-10-5p, ame-miR-100-5p, ame-miR-1000-5p, ame-miR-1006-3p, ame-miR-11-3p, ame-miR-1175-3p, ame-miR-12-5p, ame-miR-124-3p, ame-miR-125-5p, ame-miR-133-3p, ame-miR-137-3p, ame-miR-13a-3p, ame-miR-13b-3p, ame-miR-14-3p, ame-miR-184-3p, ame-miR-190-5p, ame-miR-193-3p, ame-miR-2-3p, ame-miR-210-3p, ame-miR-219-5p, ame-miR-252a-5p, ame-miR-252b-5p, ame-miR-263a-5p, ame-miR-263b-5p, ame-miR-275-3p, ame-miR-276-3p, ame-miR-2765-3p, ame-miR-2765-5p, ame-miR-277-3p, ame-miR-278-3p, ame-miR-2788-3p, ame-miR-2796-3p, ame-miR-279a-3p, ame-miR-279b-3p, ame-miR-279c-3p, ame-miR-279d-3p, ame-miR-281-3p, ame-miR-282-5p, ame-miR-283-5p, ame-miR-2944-3p, ame-miR-29b-3p, ame-miR-2b-5p, ame-miR-3049-3p, ame-miR-3049-5p, ame-miR-305-5p, ame-miR-306-5p, ame-miR-307-3p, ame-miR-315-5p, ame-miR-316-5p, ame-miR-317-3p, ame-miR-318-3p, ame-miR-31a-5p, ame-miR-33-5p, ame-miR-34-5p, ame-miR-3477-5p, ame-miR-3478-3p, ame-miR-3715-5p, ame-miR-3716a-3p, ame-miR-3716b-3p, ame-miR-3717-5p, ame-miR-3718a-3p, ame-miR-3718b-5p, ame-miR-3718c-5p, ame-miR-3719-3p, ame-miR-3720-5p, ame-miR-3721-3p, ame-miR-3722-5p, ame-miR-3723-5p, ame-miR-3724-3p, ame-miR-3725-3p, ame-miR-3726-5p, ame-miR-3727-3p, ame-miR-3728-5p, ame-miR-3729-3p, ame-miR-3730-5p, ame-miR-3731-5p, ame-miR-3732-3p, ame-miR-3733-5p, ame-miR-3734-3p, ame-miR-3735-5p, ame-miR-3736-5p, ame-miR-3737-5p, ame-miR-3738-5p, ame-miR-3739-3p, ame-miR-3740-5p, ame-miR-3741-3p, ame-miR-3742-5p, ame-miR-3743-3p, ame-miR-3744-5p, ame-miR-3745-3p, ame-miR-3746-3p, ame-miR-3747a-5p, ame-miR-3747b-5p, ame-miR-3748-3p, ame-miR-3749-3p, ame-miR-375-3p, ame-miR-3750-5p, ame-miR-3751-3p, ame-miR-3752-3p, ame-miR-3753-5p, ame-miR-3754-5p, ame-miR-3755-5p, ame-miR-3756-5p, ame-miR-3757-3p, ame-miR-3758-5p, ame-miR-3759-3p, ame-miR-3760-5p, ame-miR-3761-5p, ame-miR-3762-5p, ame-miR-3763-5p, ame-miR-3764-3p, ame-miR-3765-5p, ame-miR-3766-3p, ame-miR-3767-3p, ame-miR-3768-3p, ame-miR-3769-3p, ame-miR-3770-5p, ame-miR-3771-3p, ame-miR-3772-3p, ame-miR-3773-3p, ame-miR-3774-3p, ame-miR-3775-5p, ame-miR-3776-5p, ame-miR-3777-3p, ame-miR-3778-3p, ame-miR-3779-5p, ame-miR-3780-5p, ame-miR-3781-3p, ame-miR-3782-5p, ame-miR-3783-3p, ame-miR-3784-3p, ame-miR-3785-3p, ame-miR-3786-3p, ame-miR-3786-5p, ame-miR-3787-5p, ame-miR-3788-5p, ame-miR-3789-3p, ame-miR-3790-5p, ame-miR-3791-3p, ame-miR-3792-5p, ame-miR-3793-5p, ame-miR-3794-5p, ame-miR-3795-5p, ame-miR-3796-3p, ame-miR-3797-5p, ame-miR-3798-3p, ame-miR-3799-5p, ame-miR-3800-3p, ame-miR-3801-5p, ame-miR-6000a-3p, ame-miR-6000a-5p, ame-miR-6000b-3p, ame-miR-6001-3p, ame-miR-6001-5p, ame-miR-6002-3p, ame-miR-6002-5p, ame-miR-6003-3p, ame-miR-6003-5p, ame-miR-6004-3p, ame-miR-6004-5p, ame-miR-6005-3p, ame-miR-6005-5p, ame-miR-6006-3p, ame-miR-6006-5p, ame-miR-6012-3p, ame-miR-6037-3p, ame-miR-6038-5p, ame-miR-6039-5p, ame-miR-6040-3p, ame-miR-6041-3p, ame-miR-6042-3p, ame-miR-6043-3p, ame-miR-6044-5p, ame-miR-6045-5p, ame-miR-6046-3p, ame-miR-6047a-3p, ame-miR-6047a-5p, ame-miR-6047b-3p, ame-miR-6048-3p, ame-miR-6049-5p, ame-miR-6050-5p, ame-miR-6051-3p, ame-miR-6052-5p, ame-miR-6053-5p, ame-miR-6054-3p, ame-miR-6055-5p, ame-miR-6056-5p, ame-miR-6057-5p, ame-miR-6058-5p, ame-miR-6059-3p, ame-miR-6060-5p, ame-miR-6061-3p, ame-miR-6062-3p, ame-miR-6063-3p, ame-miR-6064-5p, ame-miR-6065-3p, ame-miR-6066-5p, ame-miR-6067-5p, ame-miR-7-5p, ame-miR-71-5p, ame-miR-750-3p, ame-miR-79-3p, ame-miR-8-3p, ame-miR-87-3p, ame-miR-927a-5p, ame-miR-927b-5p, ame-miR-928-5p, ame-miR-929-5p, ame-miR-92a-3p, ame-miR-92b-3p, ame-miR-92c-3p, ame-miR-92c-5p, ame-miR-932-5p, ame-miR-965-3p, ame-miR-965-5p, ame-miR-971-3p, ame-miR-980-3p, ame-miR-981-3p, ame-miR-985-3p, ame-miR-9864-5p, ame-miR-9865-5p, ame-miR-9866-5p, ame-miR-9867-5p, ame-miR-9868-3p, ame-miR-9869-5p, ame-miR-9870-5p, ame-miR-9871-3p, ame-miR-9872-5p, ame-miR-9873-3p, ame-miR-9874-5p, ame-miR-9875-3p, ame-miR-9876-3p, ame-miR-9877-3p, ame-miR-9878-3p, ame-miR-9879-3p, ame-miR-9880-3p, ame-miR-9881-5p, ame-miR-9882-5p, ame-miR-9883-5p, ame-miR-9884-3p, ame-miR-9885-5p, ame-miR-9886-3p, ame-miR-9887-5p, ame-miR-9888-5p, ame-miR-9889-3p, ame-miR-989-3p, ame-miR-9890-5p, ame-miR-9891-3p, ame-miR-9892-3p, ame-miR-9893-5p, ame-miR-9894-3p, ame-miR-9895-3p, ame-miR-9896-5p, ame-miR-993-3p, ame-miR-996-3p, ame-miR-9a-5p, ame-miR-9b-5p, ame-miR-9c-3p, ame-miR-iab-4-5p, amg-miR2086, amg-miR319, amg-miR396, ami-let-7a-2-3p, ami-let-7a-3p, ami-let-7a-4-3p, ami-let-7a-5p, ami-let-7b-3p, ami-let-7b-5p, ami-let-7c-2-3p, ami-let-7c-3p, ami-let-7c-5p, ami-let-7d-3p, ami-let-7d-5p, ami-let-7e-3p, ami-let-7e-5p, ami-let-7f-3p, ami-let-7f-5p, ami-let-7g-3p, ami-let-7g-5p, ami-let-7i-3p, ami-let-7i-5p, ami-miR-100-3p, ami-miR-100-5p, ami-miR-101-2-5p, ami-miR-101-3p, ami-miR-101-5p, ami-miR-103-3p, ami-miR-106-5p, ami-miR-107-3p, ami-miR-107-5p, ami-miR-10a-3p, ami-miR-10a-5p, ami-miR-10b-3p, ami-miR-10b-5p, ami-miR-10c-3p, ami-miR-10c-5p, ami-miR-122-3p, ami-miR-122-5p, ami-miR-124-3p, ami-miR-124-5p, ami-miR-125a-5p, ami-miR-125b-2-3p, ami-miR-125b-3p, ami-miR-125b-5p, ami-miR-126-3p, ami-miR-126-5p, ami-miR-128-2-5p, ami-miR-128-3p, ami-miR-128-5p, ami-miR-129a-3p, ami-miR-129a-5p, ami-miR-129b-3p, ami-miR-129b-5p, ami-miR-1306-3p, ami-miR-1306-5p, ami-miR-130a-3p, ami-miR-130b-3p, ami-miR-130b-5p, ami-miR-130c-3p, ami-miR-132-3p, ami-miR-132-5p, ami-miR-1329-5p, ami-miR-133a-3p, ami-miR-133a-5p, ami-miR-133b-3p, ami-miR-135-2-3p, ami-miR-135-3-3p, ami-miR-135-3p, ami-miR-135-5p, ami-miR-137a-3p, ami-miR-137b-3p, ami-miR-138-2-3p, ami-miR-138-3p, ami-miR-138-5p, ami-miR-1388-3p, ami-miR-1388-5p, ami-miR-139-3p, ami-miR-139-5p, ami-miR-1397-3p, ami-miR-1397-5p, ami-miR-140-3p, ami-miR-140-5p, ami-miR-1416-5p, ami-miR-142-2-5p, ami-miR-142-3p, ami-miR-142-5p, ami-miR-143-3p, ami-miR-143-5p, ami-miR-144-3p, ami-miR-144-5p, ami-miR-145-3p, ami-miR-145-5p, ami-miR-146a-5p, ami-miR-146b-3p, ami-miR-146b-5p, ami-miR-146c-3p, ami-miR-146c-5p, ami-miR-147-3p, ami-miR-147-5p, ami-miR-148b-3p, ami-miR-148b-5p, ami-miR-150-3p, ami-miR-150-5p, ami-miR-152-3p, ami-miR-152-5p, ami-miR-153-3p, ami-miR-155-5p, ami-miR-15a-3p, ami-miR-15a-5p, ami-miR-15b-3p, ami-miR-15b-5p, ami-miR-15c-3p, ami-miR-15c-5p, ami-miR-1641-5p, ami-miR-1662-5p, ami-miR-1677a-3p, ami-miR-1677b-3p, ami-miR-1677b-5p, ami-miR-1677c-3p, ami-miR-1677c-5p, ami-miR-16a-2-3p, ami-miR-16a-3p, ami-miR-16a-5p, ami-miR-16b-3p, ami-miR-16b-5p, ami-miR-17-3p, ami-miR-17-5p, ami-miR-1720-3p, ami-miR-1788-3p, ami-miR-1788-5p, ami-miR-1791-3p, ami-miR-1791-5p, ami-miR-18-3p, ami-miR-18-5p, ami-miR-1805-3p, ami-miR-1805-5p, ami-miR-181a-2-3p, ami-miR-181a-3-3p, ami-miR-181a-3p, ami-miR-181a-5p, ami-miR-181b-2-3p, ami-miR-181b-3-3p, ami-miR-181b-3p, ami-miR-181b-5p, ami-miR-182-3p, ami-miR-182-5p, ami-miR-183-3p, ami-miR-183-5p, ami-miR-184-3p, ami-miR-184-5p, ami-miR-187-3p, ami-miR-187-5p, ami-miR-190a-3p, ami-miR-190a-5p, ami-miR-190b-5p, ami-miR-191-5p, ami-miR-192-3p, ami-miR-192-5p, ami-miR-193a-3p, ami-miR-193a-5p, ami-miR-193b-3p, ami-miR-193b-5p, ami-miR-194-2-3p, ami-miR-194-3p, ami-miR-194-5p, ami-miR-196-5p, ami-miR-199-3p, ami-miR-199-5p, ami-miR-19a-5p, ami-miR-19b-3p, ami-miR-19b-5p, ami-miR-1a-2-5p, ami-miR-1a-3p, ami-miR-1a-5p, ami-miR-1b-3p, ami-miR-1b-5p, ami-miR-200a-3p, ami-miR-200a-5p, ami-miR-200b-3p, ami-miR-200b-5p, ami-miR-202-5p, ami-miR-203-3p, ami-miR-203-5p, ami-miR-204-2-3p, ami-miR-204-3p, ami-miR-204-5p, ami-miR-205a-3p, ami-miR-205a-5p, ami-miR-205b-5p, ami-miR-206-3p, ami-miR-206-5p, ami-miR-208-3p, ami-miR-20a-3p, ami-miR-20a-5p, ami-miR-21-3p, ami-miR-21-5p, ami-miR-210-3p, ami-miR-210-5p, ami-miR-212-3p, ami-miR-212-5p, ami-miR-214-3p, ami-miR-214-5p, ami-miR-215-3p, ami-miR-215-5p, ami-miR-216a-5p, ami-miR-216b-3p, ami-miR-216b-5p, ami-miR-217-3p, ami-miR-217-5p, ami-miR-218-5p, ami-miR-2184-3p, ami-miR-2184-5p, ami-miR-2188-3p, ami-miR-2188-5p, ami-miR-219a-3p, ami-miR-219a-5p, ami-miR-219b-3p, ami-miR-219b-5p, ami-miR-221-3p, ami-miR-221-5p, ami-miR-222a-3p, ami-miR-222a-5p, ami-miR-222b-3p, ami-miR-222b-5p, ami-miR-223-3p, ami-miR-223-5p, ami-miR-23a-3p, ami-miR-23b-3p, ami-miR-23b-5p, ami-miR-24-2-5p, ami-miR-24-3p, ami-miR-24-5p, ami-miR-26-2-3p, ami-miR-26-3p, ami-miR-26-5p, ami-miR-27a-3p, ami-miR-27a-5p, ami-miR-27b-3p, ami-miR-27b-5p, ami-miR-2970-3p, ami-miR-2970-5p, ami-miR-2984-3p, ami-miR-29a-2-5p, ami-miR-29a-3p, ami-miR-29a-5p, ami-miR-29b-2-5p, ami-miR-29b-3p, ami-miR-29b-5p, ami-miR-301a-3p, ami-miR-301a-5p, ami-miR-301b-3p, ami-miR-301b-5p, ami-miR-302a-3p, ami-miR-302b-3p, ami-miR-302c-3p, ami-miR-302d-3p, ami-miR-3064-3p, ami-miR-30a-3p, ami-miR-30a-5p, ami-miR-30b-3p, ami-miR-30b-5p, ami-miR-30c-2-3p, ami-miR-30c-3p, ami-miR-30c-5p, ami-miR-30d-3p, ami-miR-30d-5p, ami-miR-30e-3p, ami-miR-30e-5p, ami-miR-31-3p, ami-miR-31-5p, ami-miR-32-3p, ami-miR-32-5p, ami-miR-33-2-3p, ami-miR-33-3p, ami-miR-33-5p, ami-miR-338-2-5p, ami-miR-338-3p, ami-miR-338-5p, ami-miR-34a-3p, ami-miR-34a-5p, ami-miR-34b-3p, ami-miR-34b-5p, ami-miR-34c-3p, ami-miR-34c-5p, ami-miR-3618-3p, ami-miR-365-2-5p, ami-miR-365-3p, ami-miR-365-5p, ami-miR-367-3p, ami-miR-375-3p, ami-miR-383-5p, ami-miR-425-3p, ami-miR-425-5p, ami-miR-429-3p, ami-miR-429-5p, ami-miR-449a-5p, ami-miR-449b-5p, ami-miR-449c-5p, ami-miR-449d-5p, ami-miR-454-3p, ami-miR-454-5p, ami-miR-455-3p, ami-miR-455-5p, ami-miR-456-3p, ami-miR-458-3p, ami-miR-459-5p, ami-miR-460a-5p, ami-miR-460b-3p, ami-miR-460b-5p, ami-miR-489-3p, ami-miR-489-5p, ami-miR-490-3p, ami-miR-490-5p, ami-miR-497-3p, ami-miR-497-5p, ami-miR-499-3p, ami-miR-499-5p, ami-miR-551-3p, ami-miR-551-5p, ami-miR-599-3p, ami-miR-599-5p, ami-miR-737-5p, ami-miR-7a-2-3p, ami-miR-7a-3p, ami-miR-7a-5p, ami-miR-7b-5p, ami-miR-875-5p, ami-miR-9-3p, ami-miR-9-4-3p, ami-miR-9-5p, ami-miR-92a-3p, ami-miR-92a-5p, ami-miR-92b-3p, ami-miR-93-3p, ami-miR-93-5p, ami-miR-9598-3p, ami-miR-9598-5p, ami-miR-9599-3p, ami-miR-9599-5p, ami-miR-96-3p, ami-miR-96-5p, ami-miR-9600-3p, ami-miR-9600-5p, ami-miR-9601-3p, ami-miR-9601-5p, ami-miR-9602-3p, ami-miR-9602-5p, ami-miR-9603-3p, ami-miR-9603-5p, ami-miR-9604-3p, ami-miR-9604-5p, ami-miR-9605-3p, ami-miR-9605-5p, ami-miR-9606-3p, ami-miR-9606-5p, ami-miR-9607-3p, ami-miR-9607-5p, ami-miR-9608-3p, ami-miR-9608-5p, ami-miR-9609-3p, ami-miR-9609-5p, ami-miR-9610-3p, ami-miR-9610-5p, ami-miR-9611-3p, ami-miR-9611-5p, ami-miR-9612-3p, ami-miR-9612-5p, ami-miR-9613-3p, ami-miR-9613-5p, ami-miR-98-3p, ami-miR-98-5p, ami-miR-99a-3p, ami-miR-99a-5p, ami-miR-99b-3p, ami-miR-99b-5p, aof-miR12137, aof-miR12138, aof-miR12139, aof-miR12140, aof-miR12141, aof-miR12142, aof-miR12143, aof-miR12144, aof-miR12145, aof-miR12146, aof-miR12147, aof-miR12148, aof-miR12149, aof-miR12150, aof-miR12151, aof-miR12152, aof-miR12153, aof-miR12154, aof-miR12155, aof-miR12156, aof-miR12157, aof-miR12158, aof-miR12159, aof-miR12160, aof-miR12161, aof-miR12162, aof-miR12163, aof-miR12164, aof-miR12165, aof-miR12166, aof-miR12167, aof-miR12168, aof-miR12169, aof-miR12170, aof-miR12171, aof-miR12172, aof-miR12173, aof-miR12174, aof-miR12175, aof-miR12176, aof-miR12177, aof-miR156a, aof-miR156b, aof-miR156c, aof-miR159, aof-miR160a, aof-miR160b, aof-miR160c, aof-miR164, aof-miR166a, aof-miR 166b, aof-miR166c, aof-miR166d, aof-miR167a, aof-miR167b, aof-miR 167c, aof-miR 168a, aof-miR168b, aof-miR169a, aof-miR169b, aof-miR169c, aof-miR171a, aof-miR171b, aof-miR171c, aof-miR172, aof-miR2118a, aof-miR2118b, aof-miR2118c, aof-miR2275a, aof-miR2275b, aof-miR2275c, aof-miR2275d, aof-miR319a, aof-miR319b, aof-miR390, aof-miR391, aof-miR393a, aof-miR393b, aof-miR394, aof-miR395a, aof-miR395b, aof-miR396a, aof-miR396b, aof-miR398, aof-miR399a, aof-miR399b, aof-miR399c, aof-miR408, aof-miR477a, aof-miR477b, aof-miR479, aof-miR482a, aof-miR482b, aof-miR482c, aof-miR5139a, aof-miR5139b, aof-miR535, aof-miR536, aof-miR8155, aof-miR827, aof-miR828, api-bantam, api-let-7, api-miR-1, api-miR-10, api-miR-100, api-miR-1000, api-miR-124, api-miR-137, api-miR-13a, api-miR-14, api-miR-184a, api-miR-184b, api-miR-190, api-miR-1923, api-miR-210, api-miR-219, api-miR-252a, api-miR-252b, api-miR-263a, api-miR-263b, api-miR-275, api-miR-276, api-miR-2765, api-miR-277, api-miR-278, api-miR-2796, api-miR-279a, api-miR-279b, api-miR-281, api-miR-29, api-miR-2a, api-miR-2b, api-miR-2c, api-miR-3015a, api-miR-3015b, api-miR-3015c, api-miR-3016, api-miR-3017a, api-miR-3017b, api-miR-3018, api-miR-3019, api-miR-3020, api-miR-3021, api-miR-3022, api-miR-3023, api-miR-3024, api-miR-3025, api-miR-3026, api-miR-3027, api-miR-3028, api-miR-3029, api-miR-3030, api-miR-3031, api-miR-3032, api-miR-3033, api-miR-3034, api-miR-3035, api-miR-3036, api-miR-3037, api-miR-3038, api-miR-3039, api-miR-3040, api-miR-3041, api-miR-3042, api-miR-3043, api-miR-3044, api-miR-3045a, api-miR-3045b, api-miR-3046, api-miR-3047, api-miR-3048, api-miR-3049, api-miR-3050, api-miR-3051, api-miR-3052, api-miR-3053, api-miR-3054, api-miR-3055, api-miR-3056, api-miR-306, api-miR-307, api-miR-315, api-miR-316, api-miR-317, api-miR-34, api-miR-7, api-miR-71, api-miR-8, api-miR-87a, api-miR-87b, api-miR-927, api-miR-929, api-miR-92a, api-miR-92b, api-miR-965, api-miR-971, api-miR-981, api-miR-993, api-miR-996, api-miR-998, api-miR-9a, api-miR-9b, api-miR-iab-4, apl-miR-11588-3p, apl-miR-11588-5p, apl-miR-11589-3p, apl-miR-11589-5p, apl-miR-11590-3p, apl-miR-11590-5p, apl-miR-11591-3p, apl-miR-11591-5p, aqc-miR156a, aqc-miR156b, aqc-miR159, aqc-miR160a, aqc-miR160b, aqc-miR166a, aqc-miR166b, aqc-miR166c, aqc-miR166d, aqc-miR166e, aqc-miR167, aqc-miR168, aqc-miR169a, aqc-miR169b, aqc-miR169c, aqc-miR171a, aqc-miR171b, aqc-miR171c, aqc-miR171d, aqc-miR171e, aqc-miR171f, aqc-miR172a, aqc-miR172b, aqc-miR319, aqc-miR395a, aqc-miR395b, aqc-miR396a, aqc-miR396b, aqc-miR398a, aqc-miR398b, aqc-miR399, aqc-miR408, aqc-miR477a, aqc-miR477b, aqc-miR477c, aqc-miR477d, aqc-miR477e, aqc-miR477f, aqc-miR477g, aqc-miR482a, aqc-miR482b, aqc-miR482c, aqc-miR529, aqc-miR530, aqc-miR535, aqu-miR-2014-3p, aqu-miR-2014-5p, aqu-miR-2015-3p, aqu-miR-2015-5p, aqu-miR-2016-3p, aqu-miR-2016-5p, aqu-miR-2017-3p, aqu-miR-2017-5p, aqu-miR-2018-3p, aqu-miR-2018-5p, aqu-miR-2019-3p, aqu-miR-2019-5p, aqu-miR-2020-3p, aqu-miR-2020-5p, aqu-miR-2021-3p, aqu-miR-2021-5p, asu-let-7-3p, asu-let-7-5p, asu-lin-4-3p, asu-lin-4-5p, asu-miR-1-3p, asu-miR-1-5p, asu-miR-100a-1-3p, asu-miR-100a-2-3p, asu-miR-100a-5p, asu-miR-100b-3p, asu-miR-100b-5p, asu-miR-100c-3p, asu-miR-100c-5p, asu-miR-100d-3p, asu-miR-100d-5p, asu-miR-100e-3p, asu-miR-100e-5p, asu-miR-1175-3p, asu-miR-1175-5p, asu-miR-124-3p, asu-miR-124-5p, asu-miR-133-3p, asu-miR-133-5p, asu-miR-1822-3p, asu-miR-1822-5p, asu-miR-184-3p, asu-miR-184-5p, asu-miR-228-3p, asu-miR-228-5p, asu-miR-234-3p, asu-miR-234-5p, asu-miR-236-3p, asu-miR-236-5p, asu-miR-239-3p, asu-miR-239-5p, asu-miR-250-3p, asu-miR-250-5p, asu-miR-252-3p, asu-miR-252-5p, asu-miR-279a-3p, asu-miR-279a-5p, asu-miR-279b-3p, asu-miR-279b-5p, asu-miR-279c-3p, asu-miR-279c-5p, asu-miR-283-3p, asu-miR-283-5p, asu-miR-2a-3p, asu-miR-2a-5p, asu-miR-2b-3p, asu-miR-2b-5p, asu-miR-34-3p, asu-miR-34-5p, asu-miR-36a-3p, asu-miR-36a-5p, asu-miR-36b-3p, asu-miR-36b-5p, asu-miR-36c-3p, asu-miR-36c-5p, asu-miR-36d-3p, asu-miR-36d-5p, asu-miR-36e-3p, asu-miR-36e-5p, asu-miR-36f-3p, asu-miR-36f-5p, asu-miR-375-3p, asu-miR-375-5p, asu-miR-43a-3p, asu-miR-43a-5p, asu-miR-43b-3p, asu-miR-43b-5p, asu-miR-43c-1-5p, asu-miR-43c-2-5p, asu-miR-43c-3p, asu-miR-43d-3p, asu-miR-43d-5p, asu-miR-43e-3p, asu-miR-43e-5p, asu-miR-44a-3p, asu-miR-44a-5p, asu-miR-44b-3p, asu-miR-44b-5p, asu-miR-46-3p, asu-miR-46-5p, asu-miR-49-3p, asu-miR-49-5p, asu-miR-50-3p, asu-miR-50-5p, asu-miR-5342-3p, asu-miR-5342-5p, asu-miR-5345a-3p, asu-miR-5345a-5p, asu-miR-5345b, asu-miR-5346-3p, asu-miR-5346-5p, asu-miR-5347-3p, asu-miR-5347-5p, asu-miR-5348-3p, asu-miR-5348-5p, asu-miR-5349-3p, asu-miR-5349-5p, asu-miR-5350a-3p, asu-miR-5350a-5p, asu-miR-5350b-3p, asu-miR-5350b-5p, asu-miR-5350c-3p, asu-miR-5350c-5p, asu-miR-5350d-3p, asu-miR-5350d-5p, asu-miR-5351-3p, asu-miR-5351-5p, asu-miR-5352-3p, asu-miR-5352-5p, asu-miR-5353-3p, asu-miR-5353-5p, asu-miR-5354-3p, asu-miR-5354-5p, asu-miR-5355-3p, asu-miR-5355-5p, asu-miR-5356a-3p, asu-miR-5356a-5p, asu-miR-5356b-3p, asu-miR-5356b-5p, asu-miR-5357-3p, asu-miR-5357-5p, asu-miR-5358a-3p, asu-miR-5358a-5p, asu-miR-5358b-3p, asu-miR-5358b-5p, asu-miR-5359-3p, asu-miR-5359-5p, asu-miR-5360-3p, asu-miR-5360-5p, asu-miR-5361-3p, asu-miR-5361-5p, asu-miR-5362-3p, asu-miR-5362-5p, asu-miR-5363-3p, asu-miR-5363-5p, asu-miR-5364-3p, asu-miR-5364-5p, asu-miR-5365a-3p, asu-miR-5365a-5p, asu-miR-5365b-3p, asu-miR-5365b-5p, asu-miR-5366-3p, asu-miR-5366-5p, asu-miR-5367-3p, asu-miR-5367-5p, asu-miR-56, asu-miR-57-3p, asu-miR-57-5p, asu-miR-67-3p, asu-miR-67-5p, asu-miR-7-3p, asu-miR-7-5p, asu-miR-71-3p, asu-miR-71-5p, asu-miR-72-3p, asu-miR-72-5p, asu-miR-750-3p, asu-miR-750-5p, asu-miR-76-3p, asu-miR-76-5p, asu-miR-79-3p, asu-miR-79-5p, asu-miR-791-3p, asu-miR-791-5p, asu-miR-81a, asu-miR-81b-3p, asu-miR-81b-5p, asu-miR-81c-3p, asu-miR-81c-5p, asu-miR-83-3p, asu-miR-83-5p, asu-miR-84-3p, asu-miR-84-5p, asu-miR-86-3p, asu-miR-86-5p, asu-miR-87a-3p, asu-miR-87a-5p, asu-miR-87b-3p, asu-miR-87b-5p, asu-miR-9-3p, asu-miR-9-5p, asu-miR-92-3p, asu-miR-92-5p, asu-miR-993-3p, asu-miR-993-5p, ata-miR1432-3p, ata-miR1432-5p, ata-miR156a-3p, ata-miR156a-5p, ata-miR156b-3p, ata-miR156b-5p, ata-miR156c-3p, ata-miR156c-5p, ata-miR156d-3p, ata-miR156d-5p, ata-miR156e-3p, ata-miR156e-5p, ata-miR160a-3p, ata-miR160a-5p, ata-miR160b-3p, ata-miR160b-5p, ata-miR160c-3p, ata-miR160c-5p, ata-miR164a-3p, ata-miR164a-5p, ata-miR164b-3p, ata-miR164b-5p, ata-miR164c-3p, ata-miR164c-5p, ata-miR166a-3p, ata-miR166a-5p, ata-miR166b-3p, ata-miR166b-5p, ata-miR166c-3p, ata-miR166c-5p, ata-miR166d-3p, ata-miR166d-5p, ata-miR166e-3p, ata-miR166e-5p, ata-miR167a-3p, ata-miR167a-5p, ata-miR167b-3p, ata-miR167b-5p, ata-miR167c-3p, ata-miR167c-5p, ata-miR167d-3p, ata-miR167d-5p, ata-miR167e-3p, ata-miR167e-5p, ata-miR167f-3p, ata-miR167f-5p, ata-miR168-3p, ata-miR168-5p, ata-miR169a-3p, ata-miR169a-5p, ata-miR169b-3p, ata-miR169b-5p, ata-miR169c-3p, ata-miR169c-5p, ata-miR169d-3p, ata-miR169d-5p, ata-miR169e-5p, ata-miR169f-3p, ata-miR169f-5p, ata-miR169g-3p, ata-miR169g-5p, ata-miR169 h-3p, ata-miR169 h-5p, ata-miR169i-3p, ata-miR169i-5p, ata-miR169j-3p, ata-miR169j-5p, ata-miR171a-3p, ata-miR171a-5p, ata-miR171b-3p, ata-miR171b-5p, ata-miR171c-3p, ata-miR171c-5p, ata-miR171d-3p, ata-miR171d-5p, ata-miR172a-3p, ata-miR172a-5p, ata-miR172b-3p, ata-miR172b-5p, ata-miR172c-3p, ata-miR172c-5p, ata-miR2118a-3p, ata-miR2118a-5p, ata-miR2118b-3p, ata-miR2118b-5p, ata-miR2118c-3p, ata-miR2118d-3p, ata-miR2275a-3p, ata-miR2275a-5p, ata-miR2275b-3p, ata-miR2275b-5p, ata-miR2275c-3p, ata-miR2275c-5p, ata-miR319-3p, ata-miR319-5p, ata-miR390-3p, ata-miR390-5p, ata-miR393-3p, ata-miR393-5p, ata-miR394-3p, ata-miR394-5p, ata-miR395a-3p, ata-miR395a-5p, ata-miR395b-3p, ata-miR395b-5p, ata-miR395c-3p, ata-miR395c-5p, ata-miR395d-3p, ata-miR395d-5p, ata-miR395e-3p, ata-miR395e-5p, ata-miR395f-3p, ata-miR395f-5p, ata-miR396a-3p, ata-miR396a-5p, ata-miR396b-3p, ata-miR396b-5p, ata-miR396c-3p, ata-miR396c-5p, ata-miR396d-3p, ata-miR396d-5p, ata-miR396e-3p, ata-miR396e-5p, ata-miR398f-3p, ata-miR398f-5p, ata-miR398g-3p, ata-miR398g-5p, ata-miR399a-3p, ata-miR399a-5p, ata-miR399b-3p, ata-miR399b-5p, ata-miR408-3p, ata-miR408-5p, ata-miR5062a-3p, ata-miR5062a-5p, ata-miR5062b-3p, ata-miR5062b-5p, ata-miR5070-3p, ata-miR5070-5p, ata-miR5084-3p, ata-miR5084-5p, ata-miR5168-3p, ata-miR5168-5p, ata-miR5181-3p, ata-miR5181-5p, ata-miR5200-3p, ata-miR5200-5p, ata-miR528-3p, ata-miR528-5p, ata-miR6201-3p, ata-miR6201-5p, ata-miR9672-3p, ata-miR9672-5p, ata-miR9674a-3p, ata-miR9674a-5p, ata-miR9674b-3p, ata-miR9674b-5p, ata-miR9674c-3p, ata-miR9674c-5p, ata-miR9677-3p, ata-miR9677-5p, ata-miR9772a-3p, ata-miR9772a-5p, ata-miR9772b-3p, ata-miR9772b-5p, ata-miR9776-3p, ata-miR9776-5p, ata-miR9783-3p, ata-miR9783-5p, ata-miR9863a-3p, ata-miR9863a-5p, ata-miR9863b-3p, ata-miR9863b-5p, ath-miR10515, ath-miR156a-3p, ath-miR156a-5p, ath-miR156b-3p, ath-miR156b-5p, ath-miR156c-3p, ath-miR156c-5p, ath-miR156d-3p, ath-miR156d-5p, ath-miR156e, ath-miR156f-3p, ath-miR156f-5p, ath-miR156g, ath-miR156h, ath-miR156i, ath-miR156j, ath-miR157a-3p, ath-miR157a-5p, ath-miR157b-3p, ath-miR157b-5p, ath-miR157c-3p, ath-miR157c-5p, ath-miR157d, ath-miR158a-3p, ath-miR158a-5p, ath-miR158b, ath-miR159a, ath-miR159b-3p, ath-miR159b-5p, ath-miR159c, ath-miR160a-3p, ath-miR160a-5p, ath-miR160b, ath-miR160c-3p, ath-miR160c-5p, ath-miR161.1, ath-miR161.2, ath-miR162a-3p, ath-miR162a-5p, ath-miR162b-3p, ath-miR162b-5p, ath-miR163, ath-miR164a, ath-miR164b-3p, ath-miR164b-5p, ath-miR164c-3p, ath-miR164c-5p, ath-miR165a-3p, ath-miR165a-5p, ath-miR165b, ath-miR166a-3p, ath-miR166a-5p, ath-miR166b-3p, ath-miR166b-5p, ath-miR166c, ath-miR166d, ath-miR166e-3p, ath-miR166e-5p, ath-miR166f, ath-miR166g, ath-miR167a-3p, ath-miR167a-5p, ath-miR167b, ath-miR167c-3p, ath-miR167c-5p, ath-miR167d, ath-miR168a-3p, ath-miR168a-5p, ath-miR168b-3p, ath-miR168b-5p, ath-miR169a-3p, ath-miR169a-5p, ath-miR169b-3p, ath-miR169b-5p, ath-miR169c, ath-miR169d, ath-miR169e, ath-miR169f-3p, ath-miR169f-5p, ath-miR169g-3p, ath-miR169g-5p, ath-miR169h, ath-miR169i, ath-miR169j, ath-miR169k, ath-miR1691, ath-miR169m, ath-miR169n, ath-miR170-3p, ath-miR170-5p, ath-miR171a-3p, ath-miR171a-5p, ath-miR171b-3p, ath-miR171b-5p, ath-miR171c-3p, ath-miR171c-5p, ath-miR172a, ath-miR172b-3p, ath-miR172b-5p, ath-miR172c, ath-miR172d-3p, ath-miR172d-5p, ath-miR172e-3p, ath-miR172e-5p, ath-miR173-3p, ath-miR173-5p, ath-miR1886.1, ath-miR1886.2, ath-miR1886.3, ath-miR1887, ath-miR1888a, ath-miR1888b, ath-miR2111a-3p, ath-miR2111a-5p, ath-miR2111b-3p, ath-miR2111b-5p, ath-miR2112-3p, ath-miR2112-5p, ath-miR2933a, ath-miR2933b, ath-miR2934-3p, ath-miR2934-5p, ath-miR2936, ath-miR2937, ath-miR2938, ath-miR2939, ath-miR319a, ath-miR319b, ath-miR319c, ath-miR3434-3p, ath-miR3434-5p, ath-miR3440b-3p, ath-miR3440b-5p, ath-miR390a-3p, ath-miR390a-5p, ath-miR390b-3p, ath-miR390b-5p, ath-miR391-3p, ath-miR391-5p, ath-miR3932a, ath-miR3932b-3p, ath-miR3932b-5p, ath-miR3933, ath-miR393a-3p, ath-miR393a-5p, ath-miR393b-3p, ath-miR393b-5p, ath-miR394a, ath-miR394b-3p, ath-miR394b-5p, ath-miR395a, ath-miR395b, ath-miR395c, ath-miR395d, ath-miR395e, ath-miR395f, ath-miR396a-3p, ath-miR396a-5p, ath-miR396b-3p, ath-miR396b-5p, ath-miR397a, ath-miR397b, ath-miR398a-3p, ath-miR398a-5p, ath-miR398b-3p, ath-miR398b-5p, ath-miR398c-3p, ath-miR398c-5p, ath-miR399a, ath-miR399b, ath-miR399c-3p, ath-miR399c-5p, ath-miR399d, ath-miR399e, ath-miR399f, ath-miR400, ath-miR401, ath-miR402, ath-miR403-3p, ath-miR403-5p, ath-miR404, ath-miR405a, ath-miR405b, ath-miR405d, ath-miR406, ath-miR407, ath-miR408-3p, ath-miR408-5p, ath-miR413, ath-miR414, ath-miR415, ath-miR416, ath-miR417, ath-miR418, ath-miR419, ath-miR420, ath-miR4221, ath-miR4227, ath-miR4228-3p, ath-miR4228-5p, ath-miR4239, ath-miR4240, ath-miR4243, ath-miR4245, ath-miR426, ath-miR447a-3p, ath-miR447a.2-3p, ath-miR447b, ath-miR447c-3p, ath-miR447c-5p, ath-miR472-3p, ath-miR472-5p, ath-miR5012, ath-miR5013, ath-miR5014a-3p, ath-miR5014a-5p, ath-miR5014b, ath-miR5015, ath-miR5016, ath-miR5017-3p, ath-miR5017-5p, ath-miR5018, ath-miR5019, ath-miR5020a, ath-miR5020b, ath-miR5020c, ath-miR5021, ath-miR5022, ath-miR5023, ath-miR5024-3p, ath-miR5024-5p, ath-miR5025, ath-miR5026, ath-miR5027, ath-miR5028, ath-miR5029, ath-miR5595a, ath-miR5628, ath-miR5629, ath-miR5630a, ath-miR5630b, ath-miR5631, ath-miR5632-3p, ath-miR5632-5p, ath-miR5633, ath-miR5634, ath-miR5635a, ath-miR5635b, ath-miR5635c, ath-miR5635d, ath-miR5636, ath-miR5637, ath-miR5638a, ath-miR5638b, ath-miR5639-3p, ath-miR5639-5p, ath-miR5640, ath-miR5641, ath-miR5642a, ath-miR5642b, ath-miR5643a, ath-miR5643b, ath-miR5644, ath-miR5645a, ath-miR5645b, ath-miR5645c, ath-miR5645d, ath-miR5645e, ath-miR5645f, ath-miR5646, ath-miR5647, ath-miR5648-3p, ath-miR5648-5p, ath-miR5649a, ath-miR5649b, ath-miR5650, ath-miR5651, ath-miR5652, ath-miR5653, ath-miR5654-3p, ath-miR5654-5p, ath-miR5655, ath-miR5656, ath-miR5657, ath-miR5658, ath-miR5659, ath-miR5660, ath-miR5661, ath-miR5662, ath-miR5663-3p, ath-miR5663-5p, ath-miR5664, ath-miR5665, ath-miR5666, ath-miR5995b, ath-miR5996, ath-miR5997, ath-miR5998a, ath-miR5998b, ath-miR5999, ath-miR771, ath-miR773a, ath-miR773b-3p, ath-miR773b-5p, ath-miR774a, ath-miR774b-3p, ath-miR774b-5p, ath-miR775, ath-miR776, ath-miR777, ath-miR778, ath-miR779.1, ath-miR779.2, ath-miR780.1, ath-miR780.2, ath-miR781a, ath-miR781b, ath-miR782, ath-miR8121, ath-miR8165, ath-miR8166, ath-miR8167a, ath-miR8167b, ath-miR8167c, ath-miR8167d, ath-miR8167e, ath-miR8167f, ath-miR8168, ath-miR8169, ath-miR8170-3p, ath-miR8170-5p, ath-miR8171, ath-miR8172, ath-miR8173, ath-miR8174, ath-miR8175, ath-miR8176, ath-miR8177, ath-miR8178, ath-miR8179, ath-miR8180, ath-miR8181, ath-miR8182, ath-miR8183, ath-miR8184, ath-miR822-3p, ath-miR822-5p, ath-miR823, ath-miR824-3p, ath-miR824-5p, ath-miR825, ath-miR826a, ath-miR826b, ath-miR827, ath-miR828, ath-miR829-3p.1, ath-miR829-3p.2, ath-miR829-5p, ath-miR830-3p, ath-miR830-5p, ath-miR831-3p, ath-miR831-5p, ath-miR832-3p, ath-miR832-5p, ath-miR833a-3p, ath-miR833a-5p, ath-miR833b, ath-miR834, ath-miR835-3p, ath-miR835-5p, ath-miR836, ath-miR837-3p, ath-miR837-5p, ath-miR838, ath-miR839-5p, ath-miR840-3p, ath-miR840-5p, ath-miR841a-3p, ath-miR841a-5p, ath-miR841b-3p, ath-miR841b-5p, ath-miR842, ath-miR843, ath-miR844-3p, ath-miR844-5p, ath-miR845a, ath-miR845b, ath-miR846-3p, ath-miR846-5p, ath-miR847, ath-miR848, ath-miR849, ath-miR850, ath-miR851-3p, ath-miR851-5p, ath-miR852, ath-miR853, ath-miR854a, ath-miR854b, ath-miR854c, ath-miR854d, ath-miR854e, ath-miR855, ath-miR856, ath-miR857, ath-miR858a, ath-miR858b, ath-miR859, ath-miR860, ath-miR861-3p, ath-miR861-5p, ath-miR862-3p, ath-miR862-5p, ath-miR863-3p, ath-miR863-5p, ath-miR864-3p, ath-miR864-5p, ath-miR865-3p, ath-miR865-5p, ath-miR866-3p, ath-miR866-5p, ath-miR867, ath-miR868-3p, ath-miR868-5p, ath-miR869.1, ath-miR869.2, ath-miR870-3p, ath-miR870-5p, atr-miR156a, atr-miR156b, atr-miR156c, atr-miR156d, atr-miR159, atr-miR160, atr-miR164a, atr-miR164b, atr-miR166a, atr-miR166b, atr-miR166c, atr-miR166d, atr-miR167, atr-miR168, atr-miR169a, atr-miR169b, atr-miR169c, atr-miR171a, atr-miR171b, atr-miR171c, atr-miR172, atr-miR2111, atr-miR2950, atr-miR319a, atr-miR319b, atr-miR319c, atr-miR319d, atr-miR319e, atr-miR390.1, atr-miR390.2, atr-miR393, atr-miR394, atr-miR395, atr-miR396a, atr-miR396b, atr-miR396c, atr-miR396d, atr-miR396e, atr-miR397a, atr-miR397b, atr-miR398, atr-miR477, atr-miR535, atr-miR828, atr-miR8551, atr-miR8552a, atr-miR8552b, atr-miR8552c, atr-miR8552d, atr-miR8553a, atr-miR8553b, atr-miR8554, atr-miR8555, atr-miR8556, atr-miR8557, atr-miR8558a, atr-miR8558b, atr-miR8559, atr-miR8560, atr-miR8561.1, atr-miR8561.2, atr-miR8562a, atr-miR8562b, atr-miR8562c, atr-miR8563, atr-miR8564, atr-miR8565a, atr-miR8565b, atr-miR8565c, atr-miR8565d, atr-miR8565e, atr-miR8565f, atr-miR8565g, atr-miR8565h, atr-miR8566, atr-miR8567, atr-miR8568, atr-miR8569, atr-miR8570, atr-miR8571, atr-miR8572, atr-miR8573, atr-miR8574, atr-miR8575, atr-miR8576, atr-miR8577, atr-miR8578.1, atr-miR8578.2, atr-miR8579, atr-miR8580, atr-miR8581, atr-miR8582, atr-miR8583, atr-miR8584, atr-miR8585, atr-miR8586, atr-miR8587, atr-miR8588, atr-miR8589, atr-miR8590, atr-miR8591, atr-miR8592, atr-miR8593, atr-miR8594.1, atr-miR8594.2, atr-miR8595, atr-miR8596, atr-miR8597, atr-miR8598, atr-miR8599, atr-miR8600, atr-miR8601, atr-miR8602, atr-miR8603, atr-miR8604, atr-miR8605, atr-miR8606, atr-miR8607, atr-miR8608, atr-miR8609, atr-miR8610.1, atr-miR8610.2, atr-miR8611, atr-miR8612, atr-miR8613, atr-miR8614, atr-miR8615, atr-miR8616, atr-miR8617, bbe-let-7a-2-3p, bbe-let-7a-3p, bbe-let-7a-5p, bbe-miR-1-3p, bbe-miR-1-5p, bbe-miR-100-3p, bbe-miR-100-5p, bbe-miR-10a-3p, bbe-miR-10a-5p, bbe-miR-10b-3p, bbe-miR-10b-5p, bbe-miR-10c-5p, bbe-miR-124-3p, bbe-miR-124-5p, bbe-miR-125a-3p, bbe-miR-125a-5p, bbe-miR-125b-3p, bbe-miR-129a-3p, bbe-miR-129a-5p, bbe-miR-129b-5p, bbe-miR-133-3p, bbe-miR-133-5p, bbe-miR-135a-3p, bbe-miR-135a-5p, bbe-miR-135b-5p, bbe-miR-137-3p, bbe-miR-153-3p, bbe-miR-182a-3p, bbe-miR-182a-5p, bbe-miR-182b-3p, bbe-miR-182b-5p, bbe-miR-182c-5p, bbe-miR-182d-3p, bbe-miR-183-5p, bbe-miR-184-3p, bbe-miR-184-5p, bbe-miR-190-3p, bbe-miR-190-5p, bbe-miR-193b-3p, bbe-miR-200a-3p, bbe-miR-200a-5p, bbe-miR-200b-3p, bbe-miR-200b-5p, bbe-miR-200c-3p, bbe-miR-2013-3p, bbe-miR-2056-3p, bbe-miR-2057-3p, bbe-miR-2057-5p, bbe-miR-2058-3p, bbe-miR-2058-5p, bbe-miR-2059-3p, bbe-miR-2059-5p, bbe-miR-2060a-3p, bbe-miR-2060b-3p, bbe-miR-2060c-3p, bbe-miR-2061-3p, bbe-miR-2061-5p, bbe-miR-2062-3p, bbe-miR-2062-5p, bbe-miR-2063-3p, bbe-miR-2063-5p, bbe-miR-2064-3p, bbe-miR-2064-5p, bbe-miR-2066-3p, bbe-miR-2066-5p, bbe-miR-2067-3p, bbe-miR-2067-5p, bbe-miR-2068-3p, bbe-miR-2068-5p, bbe-miR-2069-3p, bbe-miR-2070-3p, bbe-miR-2070-5p, bbe-miR-2071-3p, bbe-miR-2071-5p, bbe-miR-2072-3p, bbe-miR-2072-5p, bbe-miR-2076-3p, bbe-miR-2076-5p, bbe-miR-210-3p, bbe-miR-210-5p, bbe-miR-216-3p, bbe-miR-216-5p, bbe-miR-217-3p, bbe-miR-217-5p, bbe-miR-219-3p, bbe-miR-219-5p, bbe-miR-22-3p, bbe-miR-22-5p, bbe-miR-242-5p, bbe-miR-252a-3p, bbe-miR-252a-5p, bbe-miR-252b-3p, bbe-miR-252b-5p, bbe-miR-278-5p, bbe-miR-281-3p, bbe-miR-281-5p, bbe-miR-29a-3p, bbe-miR-29a-5p, bbe-miR-29b-3p, bbe-miR-29b-5p, bbe-miR-31-3p, bbe-miR-31-5p, bbe-miR-33-2-3p, bbe-miR-33-3p, bbe-miR-33-5p, bbe-miR-34a-5p, bbe-miR-34b-3p, bbe-miR-34b-5p, bbe-miR-34c-5p, bbe-miR-34d-5p, bbe-miR-34f-5p, bbe-miR-34g-5p, bbe-miR-34 h-5p, bbe-miR-375-3p, bbe-miR-4057-3p, bbe-miR-4856a-3p, bbe-miR-4856a-5p, bbe-miR-4857-3p, bbe-miR-4857-5p, bbe-miR-4859-3p, bbe-miR-4859-5p, bbe-miR-4860-3p, bbe-miR-4860-5p, bbe-miR-4861-3p, bbe-miR-4863-3p, bbe-miR-4863-5p, bbe-miR-4864-3p, bbe-miR-4864-5p, bbe-miR-4865-3p, bbe-miR-4865-5p, bbe-miR-4866-3p, bbe-miR-4866-5p, bbe-miR-4867-3p, bbe-miR-4868a-3p, bbe-miR-4868b-3p, bbe-miR-4868c-3p, bbe-miR-4868c-5p, bbe-miR-4869-3p, bbe-miR-4869-5p, bbe-miR-4873-5p, bbe-miR-4874-3p, bbe-miR-4874-5p, bbe-miR-4876-3p, bbe-miR-4877-3p, bbe-miR-4878-5p, bbe-miR-4879-5p, bbe-miR-4880-5p, bbe-miR-4887-5p, bbe-miR-4888-5p, bbe-miR-4889b-3p, bbe-miR-4890-5p, bbe-miR-4891-3p, bbe-miR-4891-5p, bbe-miR-4899-5p, bbe-miR-4904-3p, bbe-miR-4904-5p, bbe-miR-4928-3p, bbe-miR-7-3p, bbe-miR-7-5p, bbe-miR-705-5p, bbe-miR-71-3p, bbe-miR-71-5p, bbe-miR-9-3p, bbe-miR-9-5p, bbe-miR-92a-3p, bbe-miR-92a-5p, bbe-miR-92b-3p, bbe-miR-92b-5p, bbe-miR-92c-3p, bbe-miR-92d-3p, bbe-miR-92d-5p, bbe-miR-96-3p, bbe-miR-96-5p, bcy-miR156, bcy-miR396a, bcy-miR396b, bcy-miR529, bdi-miR1122, bdi-miR1127, bdi-miR1135, bdi-miR1139, bdi-miR1432, bdi-miR156a, bdi-miR156b-3p, bdi-miR156b-5p, bdi-miR156c, bdi-miR156d-3p, bdi-miR156d-5p, bdi-miR156e-3p, bdi-miR156e-5p, bdi-miR156f-3p, bdi-miR156f-5p, bdi-miR156g-3p, bdi-miR156g-5p, bdi-miR156 h-3p, bdi-miR156 h-5p, bdi-miR156i-3p, bdi-miR156i-5p, bdi-miR156j, bdi-miR159a-3p, bdi-miR159a-5p, bdi-miR159b-3p.1, bdi-miR159b-3p.2, bdi-miR159b-3p.3, bdi-miR159b-5p.1, bdi-miR159b-5p.2, bdi-miR159b-5p.3, bdi-miR159c, bdi-miR160a-3p, bdi-miR160a-5p, bdi-miR160b-3p, bdi-miR160b-5p, bdi-miR160c-3p, bdi-miR160c-5p, bdi-miR160d-3p, bdi-miR160d-5p, bdi-miR160e-3p, bdi-miR160e-5p, bdi-miR160f, bdi-miR162, bdi-miR164a-3p, bdi-miR164a-5p, bdi-miR164b, bdi-miR164c-3p, bdi-miR164c-5p, bdi-miR164e, bdi-miR164f, bdi-miR166a-3p, bdi-miR166a-5p, bdi-miR166b-3p, bdi-miR166b-5p, bdi-miR166c-3p, bdi-miR166c-5p, bdi-miR166d-3p, bdi-miR166d-5p, bdi-miR166e-3p, bdi-miR166e-5p, bdi-miR166f, bdi-miR166g-3p, bdi-miR166g-5p, bdi-miR166 h-3p, bdi-miR166 h-5p, bdi-miR166i-3p, bdi-miR166i-5p, bdi-miR166j, bdi-miR167a, bdi-miR167b, bdi-miR167c-3p, bdi-miR167c-5p, bdi-miR167d-3p, bdi-miR167d-5p, bdi-miR167e-3p, bdi-miR167e-5p, bdi-miR167f, bdi-miR167g, bdi-miR168-3p, bdi-miR168-5p, bdi-miR169a-3p, bdi-miR169a-5p, bdi-miR169b, bdi-miR169c-3p, bdi-miR169c-5p, bdi-miR169d, bdi-miR169e-3p, bdi-miR169e-5p, bdi-miR169f, bdi-miR169g, bdi-miR169 h-3p, bdi-miR169 h-5p, bdi-miR169i, bdi-miR169j-3p, bdi-miR169j-5p, bdi-miR169k-3p, bdi-miR169k-5p, bdi-miR1691, bdi-miR169m, bdi-miR169n, bdi-miR171a, bdi-miR171b, bdi-miR171c-3p, bdi-miR171c-5p, bdi-miR171d-3p, bdi-miR171d-5p, bdi-miR171e, bdi-miR171f, bdi-miR172a-3p, bdi-miR172a-5p, bdi-miR172b, bdi-miR172d, bdi-miR1878-3p, bdi-miR1878-5p, bdi-miR2118a, bdi-miR2118b, bdi-miR2275a, bdi-miR2275b, bdi-miR2275c, bdi-miR319a, bdi-miR319b-3p, bdi-miR319b-5p, bdi-miR390a-3p, bdi-miR390a-5p, bdi-miR393a, bdi-miR393b-3p, bdi-miR393b-5p, bdi-miR394, bdi-miR395a, bdi-miR395b, bdi-miR395c-3p, bdi-miR395c-5p, bdi-miR395d-3p, bdi-miR395d-5p, bdi-miR395e-3p, bdi-miR395e-5p, bdi-miR395f-3p, bdi-miR395f-5p, bdi-miR395g-3p, bdi-miR395g-5p, bdi-miR395 h-3p, bdi-miR395 h-5p, bdi-miR395j-3p, bdi-miR395j-5p, bdi-miR395k-3p, bdi-miR395k-5p, bdi-miR3951-3p, bdi-miR3951-5p, bdi-miR395m, bdi-miR395n-3p, bdi-miR395n-5p, bdi-miR3950-3p, bdi-miR3950-5p, bdi-miR395p-3p, bdi-miR395p-5p, bdi-miR395q, bdi-miR396a-3p, bdi-miR396a-5p, bdi-miR396b-3p, bdi-miR396b-5p, bdi-miR396c-3p, bdi-miR396c-5p, bdi-miR396d-3p, bdi-miR396d-5p, bdi-miR396e-3p, bdi-miR396e-5p, bdi-miR397a, bdi-miR397b-3p, bdi-miR397b-5p, bdi-miR398a, bdi-miR398b, bdi-miR399a, bdi-miR399b, bdi-miR399c, bdi-miR399d, bdi-miR408-3p, bdi-miR408-5p, bdi-miR437, bdi-miR444a, bdi-miR444b, bdi-miR444c, bdi-miR444d, bdi-miR5049-3p, bdi-miR5049-5p, bdi-miR5054, bdi-miR5055, bdi-miR5056, bdi-miR5057, bdi-miR5058, bdi-miR5059, bdi-miR5060, bdi-miR5061, bdi-miR5062a, bdi-miR5063, bdi-miR5064, bdi-miR5065, bdi-miR5066, bdi-miR5067, bdi-miR5068, bdi-miR5069, bdi-miR5070, bdi-miR5163a-3p, bdi-miR5163a-5p, bdi-miR5163b-3p, bdi-miR5163b-5p, bdi-miR5164, bdi-miR5165-3p, bdi-miR5165-5p, bdi-miR5166, bdi-miR5167a-3p, bdi-miR5167a-5p, bdi-miR5167b-3p, bdi-miR5167b-5p, bdi-miR5169a, bdi-miR5169b, bdi-miR5170, bdi-miR5171a, bdi-miR5171b, bdi-miR5172-3p, bdi-miR5172-5p, bdi-miR5173-3p, bdi-miR5173-5p, bdi-miR5174a, bdi-miR5174b-3p, bdi-miR5174b-5p, bdi-miR5174c-3p, bdi-miR5174c-5p, bdi-miR5174d-3p, bdi-miR5174d-5p, bdi-miR5174e-3p.1, bdi-miR5174e-3p.2, bdi-miR5174e-5p.1, bdi-miR5174e-5p.2, bdi-miR5174f, bdi-miR5175a, bdi-miR5175b, bdi-miR5176-3p, bdi-miR5176-5p, bdi-miR5177, bdi-miR5178-3p, bdi-miR5178-5p, bdi-miR5179, bdi-miR5180a, bdi-miR5180b, bdi-miR5181a-3p, bdi-miR5181a-5p, bdi-miR5181b, bdi-miR5181c-3p, bdi-miR5181c-5p, bdi-miR5181d, bdi-miR5181e, bdi-miR5182, bdi-miR5183, bdi-miR5184, bdi-miR5185a-3p, bdi-miR5185a-5p, bdi-miR5185b-3p, bdi-miR5185b-5p, bdi-miR5185c-3p, bdi-miR5185c-5p, bdi-miR5185d-3p, bdi-miR5185d-5p, bdi-miR5185e-3p, bdi-miR5185e-5p, bdi-miR5185f-3p, bdi-miR5185f-5p, bdi-miR5185g-3p, bdi-miR5185g-5p, bdi-miR5185 h-3p, bdi-miR5185 h-5p, bdi-miR5185i-3p, bdi-miR5185i-5p, bdi-miR5185j-3p, bdi-miR5185j-5p, bdi-miR5185k-3p, bdi-miR5185k-5p, bdi-miR51851-3p, bdi-miR51851-5p, bdi-miR5185m-3p, bdi-miR5185m-5p, bdi-miR5198, bdi-miR5199, bdi-miR5200a-3p, bdi-miR5200a-5p, bdi-miR5200b-3p, bdi-miR5200b-5p, bdi-miR5200c, bdi-miR5201-3p, bdi-miR5201-5p, bdi-miR5202, bdi-miR528-3p, bdi-miR528-5p, bdi-miR5281a, bdi-miR5281b, bdi-miR529-3p, bdi-miR529-5p, bdi-miR530a, bdi-miR530b, bdi-miR531, bdi-miR7707-3p, bdi-miR7707-5p, bdi-miR7708a-3p, bdi-miR7708a-5p, bdi-miR7708b-3p, bdi-miR7708b-5p, bdi-miR7709-3p, bdi-miR7709-5p, bdi-miR7710-3p, bdi-miR7710-5p, bdi-miR7711-3p.1, bdi-miR7711-3p.3, bdi-miR7711-3p.4, bdi-miR7711-5p.1, bdi-miR7711-5p.2, bdi-miR7711-5p.3, bdi-miR7711-5p.4, bdi-miR7712-3p, bdi-miR7712-5p, bdi-miR7713-3p, bdi-miR7713-5p, bdi-miR7714-3p, bdi-miR7714-5p, bdi-miR7715-3p, bdi-miR7715-5p, bdi-miR7716-3p, bdi-miR7716-5p, bdi-miR7717a-3p, bdi-miR7717a-5p, bdi-miR7717b-3p, bdi-miR7717b-5p, bdi-miR7717c-3p, bdi-miR7717c-5p, bdi-miR7718-3p, bdi-miR7718-5p, bdi-miR7719-3p, bdi-miR7719-5p, bdi-miR7720-3p, bdi-miR7720-5p, bdi-miR7721-3p, bdi-miR7721-5p, bdi-miR7722-3p, bdi-miR7722-5p, bdi-miR7723a-3p, bdi-miR7723a-5p, bdi-miR7723b-3p, bdi-miR7723b-5p, bdi-miR7724a-3p, bdi-miR7724a-5p, bdi-miR7724b-3p, bdi-miR7724b-5p, bdi-miR7725a-3p, bdi-miR7725a-5p, bdi-miR7725b-3p.1, bdi-miR7725b-3p.2, bdi-miR7725b-5p.1, bdi-miR7725b-5p.2, bdi-miR7726a-3p, bdi-miR7726a-5p, bdi-miR7726b-3p, bdi-miR7726b-5p, bdi-miR7727-3p, bdi-miR7727-5p, bdi-miR7728-3p, bdi-miR7728-5p, bdi-miR7729a-3p, bdi-miR7729a-5p, bdi-miR7729b-3p, bdi-miR7729b-5p, bdi-miR7730-3p, bdi-miR7730-5p, bdi-miR7731-3p, bdi-miR7731-5p, bdi-miR7732-3p, bdi-miR7732-5p, bdi-miR7733-3p, bdi-miR7733-5p, bdi-miR7734-3p, bdi-miR7734-5p, bdi-miR7735-3p, bdi-miR7735-5p, bdi-miR7736-3p, bdi-miR7736-5p, bdi-miR7737-3p, bdi-miR7737-5p, bdi-miR7738-3p, bdi-miR7738-5p, bdi-miR7739-3p, bdi-miR7739-5p, bdi-miR7740-3p, bdi-miR7740-5p, bdi-miR7741-3p.1, bdi-miR7741-3p.2, bdi-miR7741-5p.1, bdi-miR7741-5p.2, bdi-miR7742-3p, bdi-miR7742-5p, bdi-miR7743-3p, bdi-miR7743-5p, bdi-miR7744-3p, bdi-miR7744-5p, bdi-miR7745-3p, bdi-miR7745-5p, bdi-miR7746-3p, bdi-miR7746-5p, bdi-miR7747-3p, bdi-miR7747-5p, bdi-miR7748a-3p, bdi-miR7748a-5p, bdi-miR7748b-3p, bdi-miR7748b-5p, bdi-miR7749-3p, bdi-miR7749-5p, bdi-miR7750-3p, bdi-miR7750-5p, bdi-miR7751-3p, bdi-miR7751-5p, bdi-miR7752-3p, bdi-miR7752-5p, bdi-miR7753-3p, bdi-miR7753-5p, bdi-miR7754-3p, bdi-miR7754-5p, bdi-miR7755-3p, bdi-miR7755-5p, bdi-miR7756-3p, bdi-miR7756-5p, bdi-miR7757-3p.1, bdi-miR7757-3p.2, bdi-miR7757-5p.1, bdi-miR7757-5p.2, bdi-miR7758-3p, bdi-miR7758-5p, bdi-miR7759-3p, bdi-miR7759-5p, bdi-miR7760-3p, bdi-miR7760-5p, bdi-miR7761-3p, bdi-miR7761-5p, bdi-miR7762-3p, bdi-miR7762-5p, bdi-miR7763-3p, bdi-miR7763-5p, bdi-miR7764-3p, bdi-miR7764-5p, bdi-miR7765-3p, bdi-miR7765-5p, bdi-miR7766-3p, bdi-miR7766-5p, bdi-miR7767-3p, bdi-miR7767-5p, bdi-miR7768a-3p, bdi-miR7768a-5p, bdi-miR7768b-3p, bdi-miR7768b-5p, bdi-miR7769-3p, bdi-miR7769-5p, bdi-miR7770-3p, bdi-miR7770-5p, bdi-miR7771-3p, bdi-miR7771-5p, bdi-miR7772-3p, bdi-miR7772-5p, bdi-miR7773-3p, bdi-miR7773-5p, bdi-miR7774-3p, bdi-miR7774-5p, bdi-miR7775-3p, bdi-miR7775-5p, bdi-miR7776-3p.1, bdi-miR7776-3p.2, bdi-miR7776-5p.1, bdi-miR7776-5p.2, bdi-miR7777-3p.1, bdi-miR7777-3p.2, bdi-miR7777-5p.1, bdi-miR7777-5p.2, bdi-miR7778-3p, bdi-miR7778-5p, bdi-miR7779-3p, bdi-miR7779-5p, bdi-miR7780-3p, bdi-miR7780-5p, bdi-miR7781-3p, bdi-miR7781-5p, bdi-miR7782-3p, bdi-miR7782-5p, bdi-miR7783-3p, bdi-miR7783-5p, bdi-miR7784a-3p, bdi-miR7784a-5p, bdi-miR7784b-3p, bdi-miR7784b-5p, bdi-miR7785-3p, bdi-miR7785-5p, bdi-miR7786-3p, bdi-miR7786-5p, bdi-miR7787-3p, bdi-miR7787-5p, bdi-miR827-3p, bdi-miR827-5p, bdi-miR845, bdi-miR9480a, bdi-miR9480b, bdi-miR9481a, bdi-miR9481b, bdi-miR9482, bdi-miR9483a, bdi-miR9483b, bdi-miR9484, bdi-miR9485, bdi-miR9486a, bdi-miR9486b, bdi-miR9487, bdi-miR9488, bdi-miR9489, bdi-miR9490, bdi-miR9491, bdi-miR9492, bdi-miR9493, bdi-miR9494, bdi-miR9495, bdi-miR9496, bdi-miR9497, bdi-miR9498, bdi-miR9499, bdo-bantam, bdo-let-7, bdo-miR-1, bdo-miR-10, bdo-miR-100, bdo-miR-1000, bdo-miR-11, bdo-miR-11593, bdo-miR-11594, bdo-miR-11595, bdo-miR-11596, bdo-miR-11597, bdo-miR-12, bdo-miR-124, bdo-miR-125, bdo-miR-137, bdo-miR-13a, bdo-miR-13b, bdo-miR-14, bdo-miR-184, bdo-miR-190, bdo-miR-193, bdo-miR-210, bdo-miR-219, bdo-miR-252, bdo-miR-263a, bdo-miR-263b, bdo-miR-275, bdo-miR-276a, bdo-miR-276b, bdo-miR-281, bdo-miR-282, bdo-miR-283, bdo-miR-284, bdo-miR-286, bdo-miR-2a, bdo-miR-2a-2, bdo-miR-2b, bdo-miR-2c, bdo-miR-304, bdo-miR-305, bdo-miR-306, bdo-miR-307, bdo-miR-308, bdo-miR-309a, bdo-miR-309b, bdo-miR-315, bdo-miR-316, bdo-miR-318, bdo-miR-375, bdo-miR-4, bdo-miR-5a, bdo-miR-5b, bdo-miR-6, bdo-miR-7, bdo-miR-79, bdo-miR-8, bdo-miR-87, bdo-miR-927, bdo-miR-929, bdo-miR-92b, bdo-miR-932, bdo-miR-958, bdo-miR-970, bdo-miR-971, bdo-miR-980, bdo-miR-981, bdo-miR-988, bdo-miR-993, bdo-miR-994, bdo-miR-995, bdo-miR-996, bdo-miR-998, bdo-miR-999, bdo-miR-9b, bdo-miR-9c, bdo-miR-iab-4, bdo-miR-iab-8, bfl-let-7a-3p, bfl-let-7a-5p, bfl-let-7b, bfl-miR-1-3p, bfl-miR-1-5p, bfl-miR-100-3p, bfl-miR-100-5p, bfl-miR-10a-3p, bfl-miR-10a-5p, bfl-miR-10b, bfl-miR-10c, bfl-miR-124-3p, bfl-miR-124-5p, bfl-miR-125a-3p, bfl-miR-125a-5p, bfl-miR-125b, bfl-miR-129a-3p, bfl-miR-129a-5p, bfl-miR-129b, bfl-miR-133, bfl-miR-135a, bfl-miR-135b, bfl-miR-137, bfl-miR-153, bfl-miR-182a, bfl-miR-182b-3p, bfl-miR-182c, bfl-miR-182d, bfl-miR-183, bfl-miR-184-3p, bfl-miR-184-5p, bfl-miR-19-3p, bfl-miR-190, bfl-miR-193a, bfl-miR-193b, bfl-miR-200a, bfl-miR-200b, bfl-miR-200c, bfl-miR-2013, bfl-miR-2056, bfl-miR-2057, bfl-miR-2058-3p, bfl-miR-2058-5p, bfl-miR-2059-3p, bfl-miR-2059-5p, bfl-miR-2060, bfl-miR-2061, bfl-miR-2062-3p, bfl-miR-2062-5p, bfl-miR-2063, bfl-miR-2064, bfl-miR-2065, bfl-miR-2066, bfl-miR-2067, bfl-miR-2068-3p, bfl-miR-2068-5p, bfl-miR-2069, bfl-miR-2070-3p, bfl-miR-2070-5p, bfl-miR-2071-3p, bfl-miR-2071-5p, bfl-miR-2072-3p, bfl-miR-2072-5p, bfl-miR-2073, bfl-miR-2075, bfl-miR-2076-3p, bfl-miR-2076-5p, bfl-miR-210-3p, bfl-miR-210-5p, bfl-miR-216-3p, bfl-miR-216-5p, bfl-miR-217, bfl-miR-219, bfl-miR-22-3p, bfl-miR-22-5p, bfl-miR-242, bfl-miR-252a, bfl-miR-252b-3p, bfl-miR-252b-5p, bfl-miR-278, bfl-miR-281, bfl-miR-29a-3p, bfl-miR-29a-5p, bfl-miR-29b-3p, bfl-miR-29b-5p, bfl-miR-31-3p, bfl-miR-31-5p, bfl-miR-33, bfl-miR-34a, bfl-miR-34b-3p, bfl-miR-34b-5p, bfl-miR-34c, bfl-miR-34d, bfl-miR-34e, bfl-miR-375, bfl-miR-4057, bfl-miR-4856a, bfl-miR-4856b-3p, bfl-miR-4856b-5p, bfl-miR-4857, bfl-miR-4858-3p, bfl-miR-4858-5p, bfl-miR-4859-3p, bfl-miR-4859-5p, bfl-miR-4860-3p, bfl-miR-4860-5p, bfl-miR-4861, bfl-miR-4862-3p, bfl-miR-4862-5p, bfl-miR-4863-3p, bfl-miR-4863-5p, bfl-miR-4864-3p, bfl-miR-4864-5p, bfl-miR-4865-3p, bfl-miR-4865-5p, bfl-miR-4866-3p, bfl-miR-4866-5p, bfl-miR-4867, bfl-miR-4868a, bfl-miR-4868b, bfl-miR-4868c-3p, bfl-miR-4868c-5p, bfl-miR-4869-3p, bfl-miR-4869-5p, bfl-miR-4870, bfl-miR-4871-3p, bfl-miR-4871-5p, bfl-miR-4872a, bfl-miR-4872b, bfl-miR-4872c, bfl-miR-4873, bfl-miR-4874-3p, bfl-miR-4874-5p, bfl-miR-4875a-5p, bfl-miR-4875b-5p, bfl-miR-4875c-3p, bfl-miR-4875d-3p, bfl-miR-4876, bfl-miR-4877, bfl-miR-4878, bfl-miR-4879, bfl-miR-4880, bfl-miR-4881, bfl-miR-4882a, bfl-miR-4882b, bfl-miR-4882c, bfl-miR-4883, bfl-miR-4884, bfl-miR-4885, bfl-miR-4886, bfl-miR-4887, bfl-miR-4888, bfl-miR-4889a, bfl-miR-4889b, bfl-miR-4890, bfl-miR-4891-3p, bfl-miR-4891-5p, bfl-miR-4892, bfl-miR-4893, bfl-miR-4894, bfl-miR-4895, bfl-miR-4897, bfl-miR-4898, bfl-miR-4899, bfl-miR-4900a, bfl-miR-4900b, bfl-miR-4901, bfl-miR-4902, bfl-miR-4903, bfl-miR-4904-3p, bfl-miR-4904-5p, bfl-miR-4905, bfl-miR-4906, bfl-miR-4928, bfl-miR-7, bfl-miR-705, bfl-miR-71-3p, bfl-miR-71-5p, bfl-miR-9-3p, bfl-miR-9-5p, bfl-miR-92a, bfl-miR-92b-3p, bfl-miR-92b-5p, bfl-miR-92c, bfl-miR-92d-5p, bfl-miR-96-3p, bfl-miR-96-5p, bfv-miR-BF1-3p, bfv-miR-BF1-5p, bfv-miR-BF2-3p, bfv-miR-BF2-5p, bgy-miR156, bgy-miR396a, bgy-miR396b, bgy-miR529, bhv1-miR-B1, bhv1-miR-B10, bhv1-miR-B2, bhv1-miR-B3, bhv1-miR-B4, bhv1-miR-B5, bhv1-miR-B6-3p, bhv1-miR-B6-5p, bhv1-miR-B7, bhv1-miR-B8-3p, bhv1-miR-B8-5p, bhv1-miR-B9, bhv5-miR-B11, bhv5-miR-B12, bhv5-miR-B13, bhv5-miR-B14, bhv5-miR-B8, bib-miR-193, bib-miR-2788, bkv-miR-B1-3p, bkv-miR-B1-5p, blv-miR-B1-3p, blv-miR-B1-5p, blv-miR-B2-3p, blv-miR-B2-5p, blv-miR-B3-3p, blv-miR-B3-5p, blv-miR-B4-3p, blv-miR-B4-5p, blv-miR-B5-3p, blv-miR-B5-5p, bma-let-7, bma-lin-4, bma-miR-100a, bma-miR-100b, bma-miR-100c, bma-miR-100d, bma-miR-1175, bma-miR-124, bma-miR-133, bma-miR-153, bma-miR-1822, bma-miR-228, bma-miR-232, bma-miR-234, bma-miR-236, bma-miR-239-3p, bma-miR-239-5p, bma-miR-250, bma-miR-252, bma-miR-268, bma-miR-279, bma-miR-281-3p, bma-miR-281-5p, bma-miR-283, bma-miR-2a, bma-miR-2b-3p, bma-miR-2b-5p, bma-miR-2c, bma-miR-2d, bma-miR-2e, bma-miR-2f, bma-miR-2g-3p, bma-miR-2g-5p, bma-miR-2 h-3p, bma-miR-2 h-5p, bma-miR-2i-3p, bma-miR-2i-5p, bma-miR-307, bma-miR-34, bma-miR-36a, bma-miR-36a-3, bma-miR-36b-3p, bma-miR-36b-5p, bma-miR-36c-3p, bma-miR-36c-5p, bma-miR-36d-3p, bma-miR-36d-5p, bma-miR-45, bma-miR-49, bma-miR-50, bma-miR-5360, bma-miR-5361, bma-miR-5363-3p, bma-miR-5363-5p, bma-miR-5364, bma-miR-5365a, bma-miR-5365b, bma-miR-5366-3p, bma-miR-5366-5p, bma-miR-57, bma-miR-5838-3p, bma-miR-5838-5p, bma-miR-5839-3p, bma-miR-5839-5p, bma-miR-5840, bma-miR-5841-3p, bma-miR-5841-5p, bma-miR-5842-3p, bma-miR-5842-5p, bma-miR-5843, bma-miR-5844, bma-miR-5845, bma-miR-5846, bma-miR-5847, bma-miR-5848, bma-miR-5849, bma-miR-5851, bma-miR-5852, bma-miR-5853, bma-miR-5854, bma-miR-5855, bma-miR-5856a, bma-miR-5856b, bma-miR-5857, bma-miR-5858-3p, bma-miR-5858-5p, bma-miR-5859, bma-miR-5861, bma-miR-5862-3p, bma-miR-5862-5p, bma-miR-5863, bma-miR-5864, bma-miR-5865, bma-miR-5866, bma-miR-5867, bma-miR-5868, bma-miR-5869, bma-miR-5871, bma-miR-5872, bma-miR-5873, bma-miR-5874, bma-miR-5875, bma-miR-5876, bma-miR-5877, bma-miR-5878, bma-miR-5879a, bma-miR-5879b, bma-miR-5880a, bma-miR-5880b, bma-miR-5881a, bma-miR-5881b, bma-miR-5882a, bma-miR-5882b, bma-miR-5883, bma-miR-60, bma-miR-7, bma-miR-71, bma-miR-72, bma-miR-750, bma-miR-81, bma-miR-84-3p, bma-miR-84-5p, bma-miR-86, bma-miR-87a, bma-miR-87b, bma-miR-9-3p, bma-miR-9-5p, bma-miR-92, bma-miR-9501-3p, bma-miR-9502, bma-miR-9503, bma-miR-9504, bma-miR-9505-3p, bma-miR-9505-5p, bma-miR-9506-3p, bma-miR-9507, bma-miR-9508-5p, bma-miR-9509-3p, bma-miR-9510, bma-miR-9511-3p, bma-miR-9512, bma-miR-9513-5p, bma-miR-9514, bma-miR-9515, bma-miR-9516, bma-miR-9517-3p, bma-miR-9517-5p, bma-miR-9518, bma-miR-9519, bma-miR-9520, bma-miR-9521, bma-miR-9522, bma-miR-9523, bma-miR-9525, bma-miR-9526, bma-miR-9527, bma-miR-9528, bma-miR-9529, bma-miR-9530, bma-miR-9531, bma-miR-9532, bma-miR-9533, bma-miR-9534, bma-miR-9535, bma-miR-9536, bma-miR-9537, bmo-bantam-3p, bmo-bantam-5p, bmo-let-7-3p, bmo-let-7-5p, bmo-miR-10-3p, bmo-miR-10-5p, bmo-miR-100, bmo-miR-1000, bmo-miR-11-3p, bmo-miR-11-5p, bmo-miR-1175-3p, bmo-miR-1175-5p, bmo-miR-12, bmo-miR-124, bmo-miR-133, bmo-miR-137, bmo-miR-13a-3p, bmo-miR-13a-5p, bmo-miR-13b-3p, bmo-miR-13b-5p, bmo-miR-14-3p, bmo-miR-14-5p, bmo-miR-184-3p, bmo-miR-184-5p, bmo-miR-190-3p, bmo-miR-190-5p, bmo-miR-1923, bmo-miR-1926, bmo-miR-1a-3p, bmo-miR-1a-5p, bmo-miR-1b-3p, bmo-miR-1b-5p, bmo-miR-210, bmo-miR-252-3p, bmo-miR-252-5p, bmo-miR-263a-3p, bmo-miR-263a-5p, bmo-miR-263b-3p, bmo-miR-263b-5p, bmo-miR-2723, bmo-miR-2724, bmo-miR-2729, bmo-miR-2731, bmo-miR-2733a-3p, bmo-miR-2733a-5p, bmo-miR-2733b, bmo-miR-2733c, bmo-miR-2733d, bmo-miR-2733e, bmo-miR-2733f, bmo-miR-2733g, bmo-miR-2733h, bmo-miR-2733i-3p, bmo-miR-2733i-5p, bmo-miR-2733j, bmo-miR-2734, bmo-miR-2736, bmo-miR-2737, bmo-miR-2738, bmo-miR-2739, bmo-miR-274-3p, bmo-miR-274-5p, bmo-miR-2742, bmo-miR-2744, bmo-miR-2745, bmo-miR-2748, bmo-miR-275-3p, bmo-miR-275-5p, bmo-miR-2751, bmo-miR-2752, bmo-miR-2753, bmo-miR-2754, bmo-miR-2755-3p, bmo-miR-2755-5p, bmo-miR-2756-3p, bmo-miR-2756-5p, bmo-miR-2757-3p, bmo-miR-2757-5p, bmo-miR-2758-3p, bmo-miR-2758-5p, bmo-miR-2759, bmo-miR-276-3p, bmo-miR-276-5p, bmo-miR-2760-3p, bmo-miR-2760-5p, bmo-miR-2761-3p, bmo-miR-2761-5p, bmo-miR-2762, bmo-miR-2763-3p, bmo-miR-2763-5p, bmo-miR-2764, bmo-miR-2765, bmo-miR-2766-3p, bmo-miR-2766-5p, bmo-miR-2767, bmo-miR-2768-3p, bmo-miR-2768-5p, bmo-miR-2769, bmo-miR-277-3p, bmo-miR-277-5p, bmo-miR-2770-3p, bmo-miR-2770-5p, bmo-miR-2771-3p, bmo-miR-2771-5p, bmo-miR-2772a, bmo-miR-2772b, bmo-miR-2774a, bmo-miR-2774b, bmo-miR-2774c, bmo-miR-2775a, bmo-miR-2775b, bmo-miR-2776, bmo-miR-2777-3p, bmo-miR-2777-5p, bmo-miR-2778a-2-5p, bmo-miR-2778a-3p, bmo-miR-2778a-4-5p, bmo-miR-2778a-5p, bmo-miR-2778b-3p, bmo-miR-2778b-5p, bmo-miR-2778c-3p, bmo-miR-2778c-5p, bmo-miR-2778d-3p, bmo-miR-2778d-5p, bmo-miR-2779, bmo-miR-278-3p, bmo-miR-278-5p, bmo-miR-2780a-3p, bmo-miR-2780a-5p, bmo-miR-2780b, bmo-miR-2780c, bmo-miR-2780d, bmo-miR-2781, bmo-miR-2782, bmo-miR-2783, bmo-miR-2784, bmo-miR-2785, bmo-miR-2786, bmo-miR-2787-3p, bmo-miR-2787-5p, bmo-miR-2788, bmo-miR-2789, bmo-miR-2790-3p, bmo-miR-2790-5p, bmo-miR-2791, bmo-miR-2792-3p, bmo-miR-2792-5p, bmo-miR-2794, bmo-miR-2795, bmo-miR-2796-3p, bmo-miR-2796-5p, bmo-miR-2797a, bmo-miR-2797b, bmo-miR-2797c, bmo-miR-2797d, bmo-miR-2798, bmo-miR-2799, bmo-miR-279a, bmo-miR-279b-3p, bmo-miR-279b-5p, bmo-miR-279c-3p, bmo-miR-279c-5p, bmo-miR-279d-3p, bmo-miR-279d-5p, bmo-miR-279e, bmo-miR-2800, bmo-miR-2801, bmo-miR-2803, bmo-miR-2804-3p, bmo-miR-2804-5p, bmo-miR-2805, bmo-miR-2806, bmo-miR-2807a, bmo-miR-2807b, bmo-miR-2807c-3p, bmo-miR-2807c-5p, bmo-miR-2807d, bmo-miR-2808a-3p, bmo-miR-2808a-5p, bmo-miR-2808b, bmo-miR-2808c, bmo-miR-2808d, bmo-miR-2808e, bmo-miR-2809, bmo-miR-281-3p, bmo-miR-281-5p, bmo-miR-2810, bmo-miR-2812, bmo-miR-2813, bmo-miR-2814, bmo-miR-2816, bmo-miR-2817, bmo-miR-2818, bmo-miR-2819, bmo-miR-282-3p, bmo-miR-282-5p, bmo-miR-2820, bmo-miR-2821, bmo-miR-2822, bmo-miR-2824, bmo-miR-2825, bmo-miR-2826, bmo-miR-2827, bmo-miR-2828, bmo-miR-283-3p, bmo-miR-283-5p, bmo-miR-2830, bmo-miR-2831, bmo-miR-2832, bmo-miR-2833, bmo-miR-2833b, bmo-miR-2834, bmo-miR-2835, bmo-miR-2836, bmo-miR-2837, bmo-miR-2838, bmo-miR-2839-3p, bmo-miR-2839-5p, bmo-miR-2840, bmo-miR-2841, bmo-miR-2842, bmo-miR-2843-1-3p, bmo-miR-2843-2-3p, bmo-miR-2843-5p, bmo-miR-2845, bmo-miR-2846, bmo-miR-2847, bmo-miR-2848, bmo-miR-2849, bmo-miR-285, bmo-miR-2851, bmo-miR-2852, bmo-miR-2854, bmo-miR-2855, bmo-miR-2856-3p, bmo-miR-2856-5p, bmo-miR-2857, bmo-miR-2859, bmo-miR-2860, bmo-miR-2998, bmo-miR-2999, bmo-miR-2a-1-5p, bmo-miR-2a-2-5p, bmo-miR-2a-3p, bmo-miR-2b-3p, bmo-miR-2b-5p, bmo-miR-3000, bmo-miR-3001, bmo-miR-305-3p, bmo-miR-305-5p, bmo-miR-306a-3p, bmo-miR-306a-5p, bmo-miR-306b, bmo-miR-307-3p, bmo-miR-307-5p, bmo-miR-308-3p, bmo-miR-308-5p, bmo-miR-31-3p, bmo-miR-31-5p, bmo-miR-316-3p, bmo-miR-316-5p, bmo-miR-317-3p, bmo-miR-317-5p, bmo-miR-3203-3p, bmo-miR-3203-5p, bmo-miR-3204a-3p, bmo-miR-3204a-5p, bmo-miR-3204b, bmo-miR-3205, bmo-miR-3206, bmo-miR-3207, bmo-miR-3208, bmo-miR-3209, bmo-miR-3210, bmo-miR-3211, bmo-miR-3212, bmo-miR-3213-3p, bmo-miR-3213-5p, bmo-miR-3214, bmo-miR-3215, bmo-miR-3216, bmo-miR-3217, bmo-miR-3218, bmo-miR-3219, bmo-miR-3220, bmo-miR-3221, bmo-miR-3222, bmo-miR-3223-3p, bmo-miR-3223-5p, bmo-miR-3224, bmo-miR-3225, bmo-miR-3226, bmo-miR-3227, bmo-miR-3228, bmo-miR-3229, bmo-miR-3230, bmo-miR-3231, bmo-miR-3232, bmo-miR-3233, bmo-miR-3234, bmo-miR-3235, bmo-miR-3236, bmo-miR-3237, bmo-miR-3238, bmo-miR-3239, bmo-miR-3241, bmo-miR-3242, bmo-miR-3243, bmo-miR-3244, bmo-miR-3245, bmo-miR-3246, bmo-miR-3247, bmo-miR-3248, bmo-miR-3249, bmo-miR-3250, bmo-miR-3251, bmo-miR-3252, bmo-miR-3253, bmo-miR-3254, bmo-miR-3255, bmo-miR-3256, bmo-miR-3257-3p, bmo-miR-3257-5p, bmo-miR-3258, bmo-miR-3259, bmo-miR-3260, bmo-miR-3261, bmo-miR-3262, bmo-miR-3263, bmo-miR-3264, bmo-miR-3265, bmo-miR-3266, bmo-miR-3267a, bmo-miR-3267b, bmo-miR-3267c, bmo-miR-3268-3p, bmo-miR-3268-5p, bmo-miR-3269, bmo-miR-3270, bmo-miR-3271, bmo-miR-3272, bmo-miR-3273, bmo-miR-3274, bmo-miR-3275, bmo-miR-3276, bmo-miR-3277, bmo-miR-3278, bmo-miR-3279, bmo-miR-3280, bmo-miR-3282, bmo-miR-3283, bmo-miR-3284, bmo-miR-3285, bmo-miR-3286-3p, bmo-miR-3286-5p, bmo-miR-3287, bmo-miR-3288, bmo-miR-3289, bmo-miR-3290, bmo-miR-3291, bmo-miR-3292, bmo-miR-3293, bmo-miR-3294, bmo-miR-3295, bmo-miR-3296-3p, bmo-miR-3296-5p, bmo-miR-3297-3p, bmo-miR-3297-5p, bmo-miR-3298, bmo-miR-3299, bmo-miR-33-3p, bmo-miR-33-5p, bmo-miR-3301, bmo-miR-3302, bmo-miR-3303, bmo-miR-3304a, bmo-miR-3305, bmo-miR-3306, bmo-miR-3307, bmo-miR-3308-3p, bmo-miR-3308-5p, bmo-miR-3309, bmo-miR-3310, bmo-miR-3311, bmo-miR-3312, bmo-miR-3314, bmo-miR-3315, bmo-miR-3316, bmo-miR-3317, bmo-miR-3318, bmo-miR-3319, bmo-miR-3321, bmo-miR-3322, bmo-miR-3323, bmo-miR-3324, bmo-miR-3325, bmo-miR-3326, bmo-miR-3327-3p, bmo-miR-3327-5p, bmo-miR-3328, bmo-miR-3329, bmo-miR-3330, bmo-miR-3332, bmo-miR-3333, bmo-miR-3334, bmo-miR-3335, bmo-miR-3336, bmo-miR-3337, bmo-miR-3338-3p, bmo-miR-3338-5p, bmo-miR-3339, bmo-miR-3340, bmo-miR-3342, bmo-miR-3343, bmo-miR-3344, bmo-miR-3345, bmo-miR-3347a, bmo-miR-3347b, bmo-miR-3348, bmo-miR-3350, bmo-miR-3351, bmo-miR-3352, bmo-miR-3353, bmo-miR-3354, bmo-miR-3355, bmo-miR-3356, bmo-miR-3357, bmo-miR-3358, bmo-miR-3359, bmo-miR-3360, bmo-miR-3361, bmo-miR-3362, bmo-miR-3363, bmo-miR-3364, bmo-miR-3365, bmo-miR-3366, bmo-miR-3367, bmo-miR-3368, bmo-miR-3369, bmo-miR-3370, bmo-miR-3371, bmo-miR-3372-3p, bmo-miR-3372-5p, bmo-miR-3373-3p, bmo-miR-3373-5p, bmo-miR-3374-3p, bmo-miR-3374-5p, bmo-miR-3375-3p, bmo-miR-3375-5p, bmo-miR-3377-3p, bmo-miR-3377-5p, bmo-miR-3378-3p, bmo-miR-3378-5p, bmo-miR-3381-3p, bmo-miR-3381-5p, bmo-miR-3382-3p, bmo-miR-3382-5p, bmo-miR-3383-3p, bmo-miR-3383-5p, bmo-miR-3384-3p, bmo-miR-3384-5p, bmo-miR-3385-3p, bmo-miR-3385-5p, bmo-miR-3386-3p, bmo-miR-3386-5p, bmo-miR-3387-3p, bmo-miR-3387-5p, bmo-miR-3388-3p, bmo-miR-3388-5p, bmo-miR-3389-3p, bmo-miR-3389-5p, bmo-miR-3391-3p, bmo-miR-3391-5p, bmo-miR-3392, bmo-miR-3393, bmo-miR-3394, bmo-miR-3395, bmo-miR-3396, bmo-miR-3397, bmo-miR-3398, bmo-miR-3399, bmo-miR-34-3p, bmo-miR-34-5p, bmo-miR-3400, bmo-miR-3401, bmo-miR-3402, bmo-miR-3403, bmo-miR-3404, bmo-miR-3405, bmo-miR-3406-3p, bmo-miR-3406-5p, bmo-miR-3407, bmo-miR-3408, bmo-miR-3409, bmo-miR-3410, bmo-miR-3411, bmo-miR-3412, bmo-miR-3413, bmo-miR-3414-3p, bmo-miR-3414-5p, bmo-miR-3415, bmo-miR-3416, bmo-miR-3417, bmo-miR-3418, bmo-miR-3419, bmo-miR-3420, bmo-miR-3421, bmo-miR-3422, bmo-miR-3423, bmo-miR-3424, bmo-miR-3425, bmo-miR-3426, bmo-miR-3427, bmo-miR-3428, bmo-miR-3429, bmo-miR-3430, bmo-miR-375-3p, bmo-miR-375-5p, bmo-miR-6497-3p, bmo-miR-6497-5p, bmo-miR-6498-3p, bmo-miR-6498-5p, bmo-miR-7-3p, bmo-miR-7-5p, bmo-miR-71-3p, bmo-miR-71-5p, bmo-miR-745-3p, bmo-miR-745-5p, bmo-miR-750-3p, bmo-miR-750-5p, bmo-miR-79-3p, bmo-miR-79-5p, bmo-miR-8-3p, bmo-miR-8-5p, bmo-miR-87, bmo-miR-927-3p, bmo-miR-927-5p, bmo-miR-929-3p, bmo-miR-929-5p, bmo-miR-92a, bmo-miR-92b, bmo-miR-932, bmo-miR-965-3p, bmo-miR-965-5p, bmo-miR-970-3p, bmo-miR-970-5p, bmo-miR-989a, bmo-miR-989b, bmo-miR-993a-3p, bmo-miR-993a-5p, bmo-miR-993b-3p, bmo-miR-993b-5p, bmo-miR-998, bmo-miR-9a-3p, bmo-miR-9a-5p, bmo-miR-9b-3p, bmo-miR-9b-5p, bmo-miR-9c-3p, bmo-miR-9c-5p, bmo-miR-9d, bmo-miR-iab-4-3p, bmo-miR-iab-4-5p, bmo-miR-iab-8, bna-miR1140, bna-miR156a, bna-miR156b, bna-miR156c, bna-miR156d, bna-miR156e, bna-miR156f, bna-miR156g, bna-miR159, bna-miR160a, bna-miR160b, bna-miR160c, bna-miR160d, bna-miR161, bna-miR162a, bna-miR164a, bna-miR164b, bna-miR164c, bna-miR164d, bna-miR166a, bna-miR166b, bna-miR166c, bna-miR166d, bna-miR166e, bna-miR166f, bna-miR167a, bna-miR167b, bna-miR167c, bna-miR167d, bna-miR168a, bna-miR168b, bna-miR169a, bna-miR169b, bna-miR169c, bna-miR169d, bna-miR169e, bna-miR169f, bna-miR169g, bna-miR169h, bna-miR169i, bna-miR169j, bna-miR169k, bna-miR1691, bna-miR169m, bna-miR169n, bna-miR171a, bna-miR171b, bna-miR171c, bna-miR171d, bna-miR171e, bna-miR171f, bna-miR171g, bna-miR172a, bna-miR172b, bna-miR172c, bna-miR172d, bna-miR2111a-3p, bna-miR2111a-5p, bna-miR2111b-3p, bna-miR2111b-5p, bna-miR2111c, bna-miR2111d, bna-miR390a, bna-miR390b, bna-miR390c, bna-miR393, bna-miR394a, bna-miR394b, bna-miR395a, bna-miR395b, bna-miR395c, bna-miR395d, bna-miR395e, bna-miR395f, bna-miR396a, bna-miR397a, bna-miR397b, bna-miR399a, bna-miR399b, bna-miR399c, bna-miR403, bna-miR6028, bna-miR6029, bna-miR6030, bna-miR6031, bna-miR6032, bna-miR6033, bna-miR6034, bna-miR6035, bna-miR6036, bna-miR824, bna-miR860, bol-miR157a, bol-miR171a, bol-miR172a, bol-miR172b, bol-miR398a-3p, bol-miR398a-5p, bol-miR824, bol-miR9408, bol-miR9409, bol-miR9410, bol-miR9411, bpcv1-miR-B1, bpcv2-miR-B1, bra-miR1140, bra-miR156a-3p, bra-miR156a-5p, bra-miR156b-3p, bra-miR156b-5p, bra-miR156c-3p, bra-miR156c-5p, bra-miR156d-3p, bra-miR156d-5p, bra-miR156e-3p, bra-miR156e-5p, bra-miR156f-3p, bra-miR156f-5p, bra-miR156g-3p, bra-miR156g-5p, bra-miR157a, bra-miR158-3p, bra-miR158-5p, bra-miR159a, bra-miR160a-3p, bra-miR160a-5p, bra-miR161-3p, bra-miR161-5p, bra-miR162-3p, bra-miR162-5p, bra-miR164a, bra-miR164b-3p, bra-miR164b-5p, bra-miR164c-3p, bra-miR164c-5p, bra-miR164d-3p, bra-miR164d-5p, bra-miR164e-3p, bra-miR164e-5p, bra-miR167a, bra-miR167b, bra-miR167c, bra-miR167d, bra-miR168a-3p, bra-miR168a-5p, bra-miR168b-3p, bra-miR168b-5p, bra-miR168c-3p, bra-miR168c-5p, bra-miR171a, bra-miR171b, bra-miR171c, bra-miR171d, bra-miR171e, bra-miR172a, bra-miR172b-3p, bra-miR172b-5p, bra-miR172c-3p, bra-miR172c-5p, bra-miR172d-3p, bra-miR172d-5p, bra-miR1885a, bra-miR1885b, bra-miR2111-3p, bra-miR2111-5p, bra-miR2111a-3p, bra-miR2111a-5p, bra-miR2111b-3p, bra-miR2111b-5p, bra-miR319-3p, bra-miR319-5p, bra-miR390-3p, bra-miR390-5p, bra-miR391-3p, bra-miR391-5p, bra-miR395a-3p, bra-miR395a-5p, bra-miR395b-3p, bra-miR395b-5p, bra-miR395c-3p, bra-miR395c-5p, bra-miR395d-3p, bra-miR395d-5p, bra-miR396-3p, bra-miR396-5p, bra-miR398-3p, bra-miR398-5p, bra-miR400-3p, bra-miR400-5p, bra-miR403-3p, bra-miR403-5p, bra-miR408-3p, bra-miR408-5p, bra-miR5654a, bra-miR5654b, bra-miR5711, bra-miR5712, bra-miR5713, bra-miR5714, bra-miR5715, bra-miR5716, bra-miR5717, bra-miR5718, bra-miR5719, bra-miR5720, bra-miR5721, bra-miR5722, bra-miR5723, bra-miR5724, bra-miR5725, bra-miR5726, bra-miR6032-3p, bra-miR6032-5p, bra-miR824, bra-miR860-3p, bra-miR860-5p, bra-miR9408-3p, bra-miR9408-5p, bra-miR9552a-3p, bra-miR9552a-5p, bra-miR9552b-3p, bra-miR9552b-5p, bra-miR9553-3p, bra-miR9553-5p, bra-miR9554-3p, bra-miR9554-5p, bra-miR9555a-3p, bra-miR9555a-5p, bra-miR9555b-3p, bra-miR9555b-5p, bra-miR9556-3p, bra-miR9556-5p, bra-miR9557-3p, bra-miR9557-5p, bra-miR9558-3p, bra-miR9558-5p, bra-miR9559-3p, bra-miR9559-5p, bra-miR9560a-3p, bra-miR9560a-5p, bra-miR9560b-3p, bra-miR9560b-5p, bra-miR9561-3p, bra-miR9561-5p, bra-miR9562-3p, bra-miR9562-5p, bra-miR9563a-3p, bra-miR9563a-5p, bra-miR9563b-3p, bra-miR9563b-5p, bra-miR9564-3p, bra-miR9564-5p, bra-miR9565-3p, bra-miR9565-5p, bra-miR9566-3p, bra-miR9566-5p, bra-miR9567-3p, bra-miR9567-5p, bra-miR9568-3p, bra-miR9568-5p, bra-miR9569-3p, bra-miR9569-5p, bta-let-7a-3p, bta-let-7a-5p, bta-let-7b, bta-let-7c, bta-let-7d, bta-let-7e, bta-let-7f, bta-let-7g, bta-let-7i, bta-miR-1, bta-miR-100, bta-miR-10020, bta-miR-101, bta-miR-10161-5p, bta-miR-10162-5p, bta-miR-10163-3p, bta-miR-10164-3p, bta-miR-10165-5p, bta-miR-10166-5p, bta-miR-10167-3p, bta-miR-10168-5p, bta-miR-10169-3p, bta-miR-10170-5p, bta-miR-10171-3p, bta-miR-10172-3p, bta-miR-10172-5p, bta-miR-10173-5p, bta-miR-10174-3p, bta-miR-10174-5p, bta-miR-10175-3p, bta-miR-10175-5p, bta-miR-10176-5p, bta-miR-10177-5p, bta-miR-10178-5p, bta-miR-10179-5p, bta-miR-10180-3p, bta-miR-10181-5p, bta-miR-10182-3p, bta-miR-10182-5p, bta-miR-10183-5p, bta-miR-10184-3p, bta-miR-10185-5p, bta-miR-10225a, bta-miR-10225b, bta-miR-103, bta-miR-105a, bta-miR-105b, bta-miR-106a, bta-miR-106b, bta-miR-107, bta-miR-10a, bta-miR-10b, bta-miR-1179, bta-miR-1185, bta-miR-1193, bta-miR-1197, bta-miR-11971, bta-miR-11972, bta-miR-11973, bta-miR-11974, bta-miR-11975, bta-miR-11976, bta-miR-11977, bta-miR-11978, bta-miR-11979, bta-miR-11980, bta-miR-11981, bta-miR-11982, bta-miR-11983, bta-miR-11984, bta-miR-11985, bta-miR-11986, bta-miR-11986b, bta-miR-11986c, bta-miR-11987, bta-miR-11988, bta-miR-11989, bta-miR-11990, bta-miR-11991, bta-miR-11992, bta-miR-11993, bta-miR-11994, bta-miR-11995, bta-miR-11996, bta-miR-11997, bta-miR-11998, bta-miR-11999, bta-miR-12000, bta-miR-12001, bta-miR-12002a, bta-miR-12002b, bta-miR-12003, bta-miR-12004, bta-miR-12005, bta-miR-12006, bta-miR-12007, bta-miR-12008, bta-miR-12009, bta-miR-12010, bta-miR-12011, bta-miR-12012, bta-miR-12013, bta-miR-12014, bta-miR-12015, bta-miR-12016, bta-miR-12017, bta-miR-12018, bta-miR-12019, bta-miR-12020, bta-miR-12021, bta-miR-12022, bta-miR-12023, bta-miR-12024, bta-miR-12025, bta-miR-12026, bta-miR-12027, bta-miR-12028, bta-miR-12029, bta-miR-12030, bta-miR-12031, bta-miR-12032, bta-miR-12033, bta-miR-12034, bta-miR-12035, bta-miR-12036, bta-miR-12037, bta-miR-12038, bta-miR-12039, bta-miR-12040, bta-miR-12041, bta-miR-12042, bta-miR-12043, bta-miR-12044, bta-miR-12045, bta-miR-12046, bta-miR-12047, bta-miR-12048, bta-miR-12049, bta-miR-12050, bta-miR-12051, bta-miR-12052, bta-miR-12053, bta-miR-12054, bta-miR-12055, bta-miR-12056, bta-miR-12057, bta-miR-12058, bta-miR-12059, bta-miR-12060, bta-miR-12061, bta-miR-12062, bta-miR-12063, bta-miR-12064, bta-miR-122, bta-miR-1224, bta-miR-1225-3p, bta-miR-1246, bta-miR-1247-3p, bta-miR-1247-5p, bta-miR-1248, bta-miR-1249, bta-miR-124a, bta-miR-124b, bta-miR-1251, bta-miR-1256, bta-miR-125a, bta-miR-125b, bta-miR-126-3p, bta-miR-126-5p, bta-miR-1260b, bta-miR-1264, bta-miR-127, bta-miR-1271, bta-miR-1277, bta-miR-128, bta-miR-1281, bta-miR-1282, bta-miR-1284, bta-miR-1287, bta-miR-129, bta-miR-129-3p, bta-miR-129-5p, bta-miR-1291, bta-miR-1296, bta-miR-1298, bta-miR-1301, bta-miR-1306, bta-miR-1307, bta-miR-130a, bta-miR-130b, bta-miR-132, bta-miR-133a, bta-miR-133b, bta-miR-133c, bta-miR-134, bta-miR-1343-3p, bta-miR-1343-5p, bta-miR-135a, bta-miR-135b, bta-miR-136, bta-miR-137, bta-miR-138, bta-miR-1388-3p, bta-miR-1388-5p, bta-miR-139, bta-miR-140, bta-miR-141, bta-miR-142-3p, bta-miR-142-5p, bta-miR-143, bta-miR-1434-3p, bta-miR-1434-5p, bta-miR-144, bta-miR-145, bta-miR-1468, bta-miR-146a, bta-miR-146b, bta-miR-147, bta-miR-148a, bta-miR-148b, bta-miR-148c, bta-miR-148d, bta-miR-149-3p, bta-miR-149-5p, bta-miR-150, bta-miR-151-3p, bta-miR-151-5p, bta-miR-152, bta-miR-153, bta-miR-154a, bta-miR-154b, bta-miR-154c, bta-miR-155, bta-miR-1584-3p, bta-miR-1584-5p, bta-miR-15a, bta-miR-15b, bta-miR-1603, bta-miR-16a, bta-miR-16b, bta-miR-17-3p, bta-miR-17-5p, bta-miR-1721, bta-miR-1777a, bta-miR-1777b, bta-miR-1814a, bta-miR-1814b, bta-miR-1814c, bta-miR-181a, bta-miR-181b, bta-miR-181c, bta-miR-181d, bta-miR-182, bta-miR-183, bta-miR-1835, bta-miR-1839, bta-miR-184, bta-miR-1842, bta-miR-1843, bta-miR-185, bta-miR-186, bta-miR-187, bta-miR-188, bta-miR-18a, bta-miR-18b, bta-miR-190a, bta-miR-190b, bta-miR-191, bta-miR-1911, bta-miR-191b, bta-miR-192, bta-miR-193a, bta-miR-193a-3p, bta-miR-193a-5p, bta-miR-193b, bta-miR-194, bta-miR-1949, bta-miR-194b, bta-miR-194b-3p, bta-miR-195, bta-miR-196a, bta-miR-196b, bta-miR-197, bta-miR-199a-3p, bta-miR-199a-5p, bta-miR-199b, bta-miR-199c, bta-miR-19a, bta-miR-19b, bta-miR-200a, bta-miR-200b, bta-miR-200c, bta-miR-202, bta-miR-204, bta-miR-205, bta-miR-206, bta-miR-208a, bta-miR-208b, bta-miR-20a, bta-miR-20b, bta-miR-21-3p, bta-miR-21-5p, bta-miR-210, bta-miR-211, bta-miR-212, bta-miR-214, bta-miR-215, bta-miR-216a, bta-miR-216b, bta-miR-217, bta-miR-218, bta-miR-219, bta-miR-219-3p, bta-miR-219-5p, bta-miR-219b-3p, bta-miR-22-3p, bta-miR-22-5p, bta-miR-221, bta-miR-222, bta-miR-223, bta-miR-224, bta-miR-2284a, bta-miR-2284aa, bta-miR-2284ab, bta-miR-2284ac, bta-miR-2284b, bta-miR-2284c, bta-miR-2284d, bta-miR-2284e, bta-miR-2284f, bta-miR-2284g, bta-miR-2284 h-3p, bta-miR-2284 h-5p, bta-miR-2284i, bta-miR-2284j, bta-miR-2284k, bta-miR-22841, bta-miR-2284m, bta-miR-2284n, bta-miR-22840, bta-miR-2284p, bta-miR-2284q, bta-miR-2284r, bta-miR-2284s, bta-miR-2284t-3p, bta-miR-2284t-5p, bta-miR-2284u, bta-miR-2284v, bta-miR-2284w, bta-miR-2284x, bta-miR-2284y, bta-miR-2284z, bta-miR-2285a, bta-miR-2285aa, bta-miR-2285ab, bta-miR-2285ac, bta-miR-2285ad, bta-miR-2285ae, bta-miR-2285af, bta-miR-2285ag-3p, bta-miR-2285ah-5p, bta-miR-2285ai-5p, bta-miR-2285aj-5p, bta-miR-2285ak-5p, bta-miR-2285al-5p, bta-miR-2285 am-5p, bta-miR-2285an, bta-miR-2285ao, bta-miR-2285ap, bta-miR-2285aq, bta-miR-2285ar, bta-miR-2285as, bta-miR-2285at, bta-miR-2285au, bta-miR-2285av, bta-miR-2285aw, bta-miR-2285ax, bta-miR-2285ay, bta-miR-2285az, bta-miR-2285b, bta-miR-2285ba, bta-miR-2285bb, bta-miR-2285bc, bta-miR-2285bd, bta-miR-2285be, bta-miR-2285bf, bta-miR-2285bg, bta-miR-2285bh, bta-miR-2285bi, bta-miR-2285bj, bta-miR-2285bk, bta-miR-2285bl, bta-miR-2285bm, bta-miR-2285bn, bta-miR-2285bo, bta-miR-2285 bp, bta-miR-2285bq, bta-miR-2285br, bta-miR-2285bs, bta-miR-2285bt, bta-miR-2285bu, bta-miR-2285bv, bta-miR-2285bw, bta-miR-2285bx, bta-miR-2285by, bta-miR-2285bz, bta-miR-2285c, bta-miR-2285ca, bta-miR-2285cb, bta-miR-2285cc, bta-miR-2285cd, bta-miR-2285ce, bta-miR-2285cf, bta-miR-2285cg, bta-miR-2285ch, bta-miR-2285ci, bta-miR-2285cj, bta-miR-2285ck, bta-miR-2285cl, bta-miR-2285 cm, bta-miR-2285cn, bta-miR-2285co, bta-miR-2285cp, bta-miR-2285cq, bta-miR-2285cr, bta-miR-2285cs, bta-miR-2285ct, bta-miR-2285cu, bta-miR-2285cv, bta-miR-2285cw, bta-miR-2285cx, bta-miR-2285cy, bta-miR-2285cz, bta-miR-2285d, bta-miR-2285da, bta-miR-2285 db, bta-miR-2285dc, bta-miR-2285dd, bta-miR-2285de, bta-miR-2285df, bta-miR-2285dg, bta-miR-2285dh, bta-miR-2285di, bta-miR-2285dj, bta-miR-2285dk, bta-miR-2285dl-3p, bta-miR-2285dm, bta-miR-2285e, bta-miR-2285f, bta-miR-2285g, bta-miR-2285h, bta-miR-2285i, bta-miR-2285j, bta-miR-2285k, bta-miR-22851, bta-miR-2285m, bta-miR-2285n, bta-miR-22850, bta-miR-2285p, bta-miR-2285q, bta-miR-2285r, bta-miR-2285s, bta-miR-2285t, bta-miR-2285u, bta-miR-2285v, bta-miR-2285w, bta-miR-2285x, bta-miR-2285y, bta-miR-2285z, bta-miR-2286, bta-miR-2287, bta-miR-2288, bta-miR-2289, bta-miR-2290, bta-miR-2291, bta-miR-2292, bta-miR-2293, bta-miR-2294, bta-miR-2295, bta-miR-2296, bta-miR-2297, bta-miR-2298, bta-miR-2299-3p, bta-miR-2299-5p, bta-miR-2300a-5p, bta-miR-2300b-3p, bta-miR-2301, bta-miR-2302, bta-miR-2303, bta-miR-2304, bta-miR-2305, bta-miR-2306, bta-miR-2307, bta-miR-2308, bta-miR-2309, bta-miR-2310, bta-miR-2311, bta-miR-2312, bta-miR-2313-3p, bta-miR-2313-5p, bta-miR-2314, bta-miR-2315, bta-miR-2316, bta-miR-2317, bta-miR-2318, bta-miR-2319a, bta-miR-2319b, bta-miR-2320-3p, bta-miR-2320-5p, bta-miR-2321, bta-miR-2322-3p, bta-miR-2322-5p, bta-miR-2323, bta-miR-2324, bta-miR-2325a, bta-miR-2325b, bta-miR-2325c, bta-miR-2326, bta-miR-2327, bta-miR-2328-3p, bta-miR-2328-5p, bta-miR-2329-3p, bta-miR-2329-5p, bta-miR-2330-3p, bta-miR-2330-5p, bta-miR-2331-3p, bta-miR-2331-5p, bta-miR-2332, bta-miR-2333, bta-miR-2334, bta-miR-2335, bta-miR-2336, bta-miR-2337, bta-miR-2338, bta-miR-2339, bta-miR-2340, bta-miR-2341, bta-miR-2342, bta-miR-2343, bta-miR-2344, bta-miR-2345, bta-miR-2346, bta-miR-2347, bta-miR-2348, bta-miR-2349, bta-miR-2350, bta-miR-2351, bta-miR-2352, bta-miR-2353, bta-miR-2354, bta-miR-2355-3p, bta-miR-2355-5p, bta-miR-2356, bta-miR-2357, bta-miR-2358, bta-miR-2359, bta-miR-2360, bta-miR-2361, bta-miR-2362, bta-miR-2363, bta-miR-2364, bta-miR-2365, bta-miR-2366, bta-miR-2367-3p, bta-miR-2367-5p, bta-miR-2368-3p, bta-miR-2368-5p, bta-miR-2369, bta-miR-2370-3p, bta-miR-2370-5p, bta-miR-2371, bta-miR-2372, bta-miR-2373-3p, bta-miR-2373-5p, bta-miR-2374, bta-miR-2375, bta-miR-2376, bta-miR-2377, bta-miR-2378, bta-miR-2379, bta-miR-2380, bta-miR-2381, bta-miR-2382-3p, bta-miR-2382-5p, bta-miR-2383, bta-miR-2384, bta-miR-2385-3p, bta-miR-2385-5p, bta-miR-2386, bta-miR-2387, bta-miR-2388-3p, bta-miR-2388-5p, bta-miR-2389, bta-miR-2390, bta-miR-2392, bta-miR-2393, bta-miR-2394, bta-miR-2395, bta-miR-2396, bta-miR-2397-3p, bta-miR-2397-5p, bta-miR-2398, bta-miR-2399-3p, bta-miR-2399-5p, bta-miR-23a, bta-miR-23b-3p, bta-miR-23b-5p, bta-miR-24, bta-miR-24-3p, bta-miR-2400, bta-miR-2401, bta-miR-2402, bta-miR-2403, bta-miR-2404, bta-miR-2405, bta-miR-2406, bta-miR-2407, bta-miR-2408, bta-miR-2409, bta-miR-2410, bta-miR-2411-3p, bta-miR-2411-5p, bta-miR-2412, bta-miR-2413, bta-miR-2414, bta-miR-2415-3p, bta-miR-2415-5p, bta-miR-2416, bta-miR-2417, bta-miR-2418, bta-miR-2419-3p, bta-miR-2419-5p, bta-miR-2420, bta-miR-2421, bta-miR-2422, bta-miR-2423, bta-miR-2424, bta-miR-2425-3p, bta-miR-2425-5p, bta-miR-2426, bta-miR-2427, bta-miR-2428, bta-miR-2429, bta-miR-2430, bta-miR-2431-3p, bta-miR-2431-5p, bta-miR-2432, bta-miR-2433, bta-miR-2434, bta-miR-2435, bta-miR-2436-3p, bta-miR-2436-5p, bta-miR-2437, bta-miR-2438, bta-miR-2439-3p, bta-miR-2439-5p, bta-miR-2440, bta-miR-2441, bta-miR-2442, bta-miR-2443, bta-miR-2444, bta-miR-2446, bta-miR-2447, bta-miR-2448-3p, bta-miR-2448-5p, bta-miR-2449, bta-miR-2450a, bta-miR-2450b, bta-miR-2450c, bta-miR-2450d, bta-miR-2451, bta-miR-2452, bta-miR-2453, bta-miR-2454-3p, bta-miR-2454-5p, bta-miR-2455, bta-miR-2456, bta-miR-2457, bta-miR-2458, bta-miR-2459, bta-miR-2460, bta-miR-2461-3p, bta-miR-2461-5p, bta-miR-2462, bta-miR-2463, bta-miR-2464-3p, bta-miR-2464-5p, bta-miR-2465, bta-miR-2466-3p, bta-miR-2466-5p, bta-miR-2467-3p, bta-miR-2467-5p, bta-miR-2468, bta-miR-2469, bta-miR-2470, bta-miR-2471-3p, bta-miR-2471-5p, bta-miR-2472, bta-miR-2473, bta-miR-2474, bta-miR-2475, bta-miR-2477, bta-miR-2478, bta-miR-2479, bta-miR-2480, bta-miR-2481, bta-miR-2482, bta-miR-2483-3p, bta-miR-2483-5p, bta-miR-2484, bta-miR-2485, bta-miR-2486-3p, bta-miR-2486-5p, bta-miR-2488, bta-miR-2489, bta-miR-25, bta-miR-26a, bta-miR-26b, bta-miR-26c, bta-miR-27a-3p, bta-miR-27a-5p, bta-miR-27b, bta-miR-28, bta-miR-2881, bta-miR-2882, bta-miR-2883, bta-miR-2885, bta-miR-2886, bta-miR-2887, bta-miR-2888, bta-miR-2889, bta-miR-2890, bta-miR-2891, bta-miR-2892, bta-miR-2893, bta-miR-2894, bta-miR-2895, bta-miR-2896, bta-miR-2897, bta-miR-2898, bta-miR-2899, bta-miR-2900, bta-miR-2901, bta-miR-2902, bta-miR-2903, bta-miR-2904, bta-miR-2917, bta-miR-296-3p, bta-miR-296-5p, bta-miR-299, bta-miR-299-2, bta-miR-29a, bta-miR-29b, bta-miR-29c, bta-miR-29d-3p, bta-miR-29d-5p, bta-miR-29e, bta-miR-301a, bta-miR-301b, bta-miR-302a, bta-miR-302b, bta-miR-302c, bta-miR-302d, bta-miR-3064, bta-miR-3065, bta-miR-30a-5p, bta-miR-30b-3p, bta-miR-30b-5p, bta-miR-30c, bta-miR-30d, bta-miR-30e-5p, bta-miR-30f, bta-miR-31, bta-miR-3120, bta-miR-3141, bta-miR-3154, bta-miR-32, bta-miR-320a, bta-miR-320b, bta-miR-323, bta-miR-323b-3p, bta-miR-324, bta-miR-326, bta-miR-328, bta-miR-329a, bta-miR-329b, bta-miR-330, bta-miR-331-3p, bta-miR-331-5p, bta-miR-335, bta-miR-338, bta-miR-339a, bta-miR-339b, bta-miR-33a, bta-miR-33b, bta-miR-340, bta-miR-342, bta-miR-3431, bta-miR-3432a, bta-miR-3432b, bta-miR-345-3p, bta-miR-345-5p, bta-miR-346, bta-miR-34a, bta-miR-34b, bta-miR-34c, bta-miR-3533, bta-miR-3578, bta-miR-3596, bta-miR-3600, bta-miR-3601, bta-miR-3602, bta-miR-3604, bta-miR-361, bta-miR-3613a, bta-miR-3613b, bta-miR-362-3p, bta-miR-362-5p, bta-miR-363, bta-miR-365-3p, bta-miR-365-5p, bta-miR-3660, bta-miR-367, bta-miR-369-3p, bta-miR-369-5p, bta-miR-370, bta-miR-371, bta-miR-374a, bta-miR-374b, bta-miR-374c, bta-miR-375, bta-miR-376a, bta-miR-376b, bta-miR-376c, bta-miR-376d, bta-miR-376e, bta-miR-377, bta-miR-378, bta-miR-378b, bta-miR-378c, bta-miR-378d, bta-miR-379, bta-miR-380-3p, bta-miR-380-5p, bta-miR-381, bta-miR-382, bta-miR-383, bta-miR-3956, bta-miR-3957, bta-miR-409a, bta-miR-409b, bta-miR-410, bta-miR-411a, bta-miR-411b, bta-miR-411c-3p, bta-miR-411c-5p, bta-miR-412, bta-miR-421, bta-miR-423-3p, bta-miR-423-5p, bta-miR-424-3p, bta-miR-424-5p, bta-miR-425-3p, bta-miR-425-5p, bta-miR-4286, bta-miR-429, bta-miR-431, bta-miR-432, bta-miR-433, bta-miR-4444, bta-miR-4449, bta-miR-448, bta-miR-449a, bta-miR-449b, bta-miR-449c, bta-miR-449d, bta-miR-450a, bta-miR-450b, bta-miR-451, bta-miR-452, bta-miR-4523, bta-miR-453, bta-miR-454, bta-miR-455-3p, bta-miR-455-5p, bta-miR-4657, bta-miR-4680, bta-miR-483, bta-miR-484, bta-miR-485, bta-miR-486, bta-miR-487a, bta-miR-487b, bta-miR-488, bta-miR-489, bta-miR-490, bta-miR-491, bta-miR-493, bta-miR-494, bta-miR-495, bta-miR-496, bta-miR-497, bta-miR-499, bta-miR-500, bta-miR-502a, bta-miR-502b, bta-miR-503-3p, bta-miR-503-5p, bta-miR-504, bta-miR-505, bta-miR-507-3p, bta-miR-507b, bta-miR-532, bta-miR-539, bta-miR-541, bta-miR-542-5p, bta-miR-543, bta-miR-544a, bta-miR-544b, bta-miR-545-3p, bta-miR-545-5p, bta-miR-551a, bta-miR-551b, bta-miR-562, bta-miR-568, bta-miR-574, bta-miR-582, bta-miR-584, bta-miR-592, bta-miR-599, bta-miR-6119-3p, bta-miR-6119-5p, bta-miR-6120-3p, bta-miR-6120-5p, bta-miR-6121-3p, bta-miR-6121-5p, bta-miR-6122-3p, bta-miR-6122-5p, bta-miR-6123, bta-miR-615, bta-miR-628, bta-miR-631, bta-miR-6501, bta-miR-6516, bta-miR-6517, bta-miR-6518, bta-miR-6519, bta-miR-652, bta-miR-6520, bta-miR-6521, bta-miR-6522, bta-miR-6523a, bta-miR-6523b, bta-miR-6524, bta-miR-6525, bta-miR-6526, bta-miR-6527, bta-miR-6528, bta-miR-6529a, bta-miR-6529b, bta-miR-653, bta-miR-6530, bta-miR-6531, bta-miR-6532, bta-miR-6533, bta-miR-6534, bta-miR-6535, bta-miR-6536, bta-miR-654, bta-miR-655, bta-miR-656, bta-miR-658, bta-miR-660, bta-miR-664a, bta-miR-664b, bta-miR-665, bta-miR-668-3p, bta-miR-669, bta-miR-670, bta-miR-671, bta-miR-6715, bta-miR-677, bta-miR-6775, bta-miR-7, bta-miR-708, bta-miR-7180, bta-miR-744, bta-miR-758, bta-miR-759, bta-miR-760-3p, bta-miR-760-5p, bta-miR-761, bta-miR-763, bta-miR-764, bta-miR-767, bta-miR-769, bta-miR-7691, bta-miR-7857-3p, bta-miR-7857-5p, bta-miR-7858, bta-miR-7859, bta-miR-7860, bta-miR-7861, bta-miR-7862, bta-miR-7863, bta-miR-7864, bta-miR-7865, bta-miR-8548, bta-miR-8549, bta-miR-8550, bta-miR-873, bta-miR-874, bta-miR-875, bta-miR-876, bta-miR-877, bta-miR-885, bta-miR-9-3p, bta-miR-9-5p, bta-miR-92a, bta-miR-92b, bta-miR-93, bta-miR-935, bta-miR-940, bta-miR-95, bta-miR-96, bta-miR-98, bta-miR-9851, bta-miR-99a-3p, bta-miR-99a-5p, bta-miR-99b, cas-miR11592, cas-miR156a, cas-miR156b-3p, cas-miR156b-5p, cas-miR156c-3p, cas-miR156c-5p, cas-miR156d-3p, cas-miR156d-5p, cas-miR156e-3p, cas-miR156e-5p, cas-miR156f-3p, cas-miR156f-5p, cas-miR156g, cas-miR156h, cas-miR156i, cas-miR156j, cas-miR156k-3p, cas-miR156k-5p, cas-miR157a, cas-miR157b, cas-miR157c, cas-miR157d, cas-miR158a, cas-miR158b, cas-miR159a, cas-miR159b-3p, cas-miR159b-5p, cas-miR159c-3p, cas-miR159c-5, cas-miR160a, cas-miR160b-3p, cas-miR160b-5p, cas-miR161, cas-miR162a, cas-miR162b, cas-miR164, cas-miR165a, cas-miR165b, cas-miR166a, cas-miR166b, cas-miR166c-3p, cas-miR166c-5p, cas-miR166d, cas-miR166e, cas-miR166f-3p, cas-miR166f-5p, cas-miR167a, cas-miR167b, cas-miR167c, cas-miR168, cas-miR169a, cas-miR169b, cas-miR169c, cas-miR169d, cas-miR169e-3p, cas-miR169e-5p, cas-miR169f, cas-miR169g-3p, cas-miR169g-5p, cas-miR170, cas-miR171a-3p, cas-miR171a-5p, cas-miR171b, cas-miR171c-3p, cas-miR171c-5p, cas-miR172a-3p, cas-miR172a-5p, cas-miR172b, cas-miR172c, cas-miR172d, cas-miR173, cas-miR2111a, cas-miR2111b, cas-miR319a, cas-miR319b, cas-miR319c, cas-miR3435, cas-miR390a-3p, cas-miR390a-5p, cas-miR390b, cas-miR393-3p, cas-miR393-5p, cas-miR394, cas-miR395a, cas-miR395b, cas-miR395c-3p, cas-miR395c-5p, cas-miR395d-3p, cas-miR395d-5p, cas-miR396a, cas-miR396b, cas-miR397, cas-miR398a-3p, cas-miR398a-5p, cas-miR398b, cas-miR399a, cas-miR399b, cas-miR400, cas-miR403, cas-miR408, cas-miR472, cas-miR5139, cas-miR8171, cas-miR825, cas-miR827a, cas-miR827b, cas-miR828, cas-miR845, cas-miR857, cas-miR858, cas-miR860, cas-miR9560, cbn-let-7, cbn-lin-4, cbn-miR-1, cbn-miR-124, cbn-miR-1822, cbn-miR-1824a, cbn-miR-1824b, cbn-miR-2, cbn-miR-228, cbn-miR-230, cbn-miR-231, cbn-miR-232, cbn-miR-233, cbn-miR-234, cbn-miR-235, cbn-miR-236, cbn-miR-237, cbn-miR-238, cbn-miR-239a, cbn-miR-239b, cbn-miR-239c, cbn-miR-239d, cbn-miR-241, cbn-miR-242, cbn-miR-244, cbn-miR-245, cbn-miR-246, cbn-miR-248, cbn-miR-249, cbn-miR-250, cbn-miR-251a, cbn-miR-251b, cbn-miR-254, cbn-miR-255, cbn-miR-259, cbn-miR-34, cbn-miR-355, cbn-miR-356, cbn-miR-357, cbn-miR-359, cbn-miR-35a, cbn-miR-35b, cbn-miR-35c, cbn-miR-35d, cbn-miR-35e, cbn-miR-35f, cbn-miR-35g, cbn-miR-35h, cbn-miR-35i, cbn-miR-35j, cbn-miR-35k, cbn-miR-351, cbn-miR-35m, cbn-miR-35n, cbn-miR-36, cbn-miR-360, cbn-miR-38, cbn-miR-39, cbn-miR-43, cbn-miR-44, cbn-miR-46, cbn-miR-47, cbn-miR-48, cbn-miR-49, cbn-miR-50, cbn-miR-51, cbn-miR-52, cbn-miR-54, cbn-miR-55, cbn-miR-56, cbn-miR-57, cbn-miR-58a, cbn-miR-58b, cbn-miR-59, cbn-miR-60, cbn-miR-61, cbn-miR-62, cbn-miR-63, cbn-miR-64a, cbn-miR-64b, cbn-miR-64c, cbn-miR-64d, cbn-miR-64e, cbn-miR-64f, cbn-miR-64g, cbn-miR-64h, cbn-miR-64i, cbn-miR-64j, cbn-miR-64k, cbn-miR-641, cbn-miR-64m, cbn-miR-67, cbn-miR-70, cbn-miR-72a, cbn-miR-72b, cbn-miR-73a, cbn-miR-73b, cbn-miR-74a, cbn-miR-74b, cbn-miR-74c, cbn-miR-74d, cbn-miR-75, cbn-miR-7579, cbn-miR-7597, cbn-miR-76, cbn-miR-7614, cbn-miR-7615, cbn-miR-7616, cbn-miR-7617, cbn-miR-7618, cbn-miR-7619, cbn-miR-7620a, cbn-miR-7620b, cbn-miR-7621a, cbn-miR-7621b, cbn-miR-7622, cbn-miR-7623, cbn-miR-7624, cbn-miR-7625, cbn-miR-7626, cbn-miR-7627, cbn-miR-7628, cbn-miR-7629, cbn-miR-7630, cbn-miR-7631, cbn-miR-7632, cbn-miR-7633, cbn-miR-7634, cbn-miR-7635, cbn-miR-7636, cbn-miR-7637, cbn-miR-7638, cbn-miR-7639, cbn-miR-7640, cbn-miR-7690, cbn-miR-77, cbn-miR-784, cbn-miR-785, cbn-miR-787, cbn-miR-789a, cbn-miR-789b, cbn-miR-79, cbn-miR-790, cbn-miR-80, cbn-miR-81, cbn-miR-82, cbn-miR-83, cbn-miR-84, cbn-miR-85, cbn-miR-86, cbn-miR-87, cbn-miR-90, cbr-let-7, cbr-lin-4, cbr-lsy-6, cbr-miR-1, cbr-miR-124a, cbr-miR-124b, cbr-miR-124c, cbr-miR-1817, cbr-miR-1822, cbr-miR-2, cbr-miR-2222, cbr-miR-2223, cbr-miR-2224, cbr-miR-2225-3p, cbr-miR-2225-5p, cbr-miR-2226-3p, cbr-miR-2226-5p, cbr-miR-2227, cbr-miR-2231, cbr-miR-228, cbr-miR-230, cbr-miR-231, cbr-miR-232, cbr-miR-233, cbr-miR-234, cbr-miR-235a, cbr-miR-235b-3p, cbr-miR-235b-5p, cbr-miR-236, cbr-miR-237-3p, cbr-miR-237-5p, cbr-miR-238, cbr-miR-239a, cbr-miR-239b, cbr-miR-240, cbr-miR-241, cbr-miR-242, cbr-miR-244, cbr-miR-245, cbr-miR-246, cbr-miR-247, cbr-miR-248, cbr-miR-249, cbr-miR-250, cbr-miR-251, cbr-miR-252, cbr-miR-253, cbr-miR-254, cbr-miR-255, cbr-miR-259, cbr-miR-268, cbr-miR-34, cbr-miR-354, cbr-miR-355, cbr-miR-356, cbr-miR-357, cbr-miR-358, cbr-miR-359, cbr-miR-35a, cbr-miR-35b, cbr-miR-35c, cbr-miR-35d-3p, cbr-miR-35d-5p, cbr-miR-35e, cbr-miR-35f, cbr-miR-35g, cbr-miR-36, cbr-miR-360, cbr-miR-38, cbr-miR-39, cbr-miR-392, cbr-miR-40, cbr-miR-41a, cbr-miR-41b, cbr-miR-42, cbr-miR-43, cbr-miR-44, cbr-miR-45, cbr-miR-46, cbr-miR-47, cbr-miR-48, cbr-miR-4813, cbr-miR-49, cbr-miR-50, cbr-miR-51, cbr-miR-52, cbr-miR-54a-3p, cbr-miR-54a-5p, cbr-miR-54b, cbr-miR-54c, cbr-miR-54d, cbr-miR-55, cbr-miR-56-3p, cbr-miR-56-5p, cbr-miR-57, cbr-miR-58a, cbr-miR-58b, cbr-miR-60, cbr-miR-61, cbr-miR-62, cbr-miR-64a, cbr-miR-64b, cbr-miR-64c, cbr-miR-65-3p, cbr-miR-65-5p, cbr-miR-67, cbr-miR-70, cbr-miR-71, cbr-miR-72, cbr-miR-73a, cbr-miR-73b, cbr-miR-74a, cbr-miR-74b-3p, cbr-miR-74b-5p, cbr-miR-75, cbr-miR-7579, cbr-miR-7580, cbr-miR-7581, cbr-miR-7582, cbr-miR-7583a-3p, cbr-miR-7583a-5p, cbr-miR-7583b, cbr-miR-7583c-3p, cbr-miR-7583c-5p, cbr-miR-7583d, cbr-miR-7584, cbr-miR-7585, cbr-miR-7586, cbr-miR-7587, cbr-miR-7588, cbr-miR-7589, cbr-miR-7590, cbr-miR-7591, cbr-miR-7592, cbr-miR-7593, cbr-miR-7594a, cbr-miR-7594b, cbr-miR-7595, cbr-miR-7596, cbr-miR-76, cbr-miR-77, cbr-miR-784, cbr-miR-785a, cbr-miR-785b, cbr-miR-786, cbr-miR-787, cbr-miR-788, cbr-miR-789, cbr-miR-79, cbr-miR-790, cbr-miR-791, cbr-miR-792, cbr-miR-80, cbr-miR-81, cbr-miR-82, cbr-miR-83, cbr-miR-84, cbr-miR-85, cbr-miR-86, cbr-miR-87, cbr-miR-90a, cbr-miR-90b-3p, cbr-miR-90b-5p, cca-miR156a, cca-miR156b, cca-miR156c, cca-miR160a, cca-miR160b, cca-miR164, cca-miR167, cca-miR168a, cca-miR169a, cca-miR169b, cca-miR171, cca-miR172, cca-miR319, cca-miR390, cca-miR393, cca-miR394, cca-miR395a, cca-miR395b, cca-miR395c, cca-miR396a-3p, cca-miR396a-5p, cca-miR396b, cca-miR396c, cca-miR398, cca-miR399, cca-miR408, cca-miR6103-3p, cca-miR6103-5p, cca-miR6104, cca-miR6105a, cca-miR6105b, cca-miR6106-3p, cca-miR6106-5p, cca-miR6107, cca-miR6108a, cca-miR6108b, cca-miR6108c, cca-miR6108d, cca-miR6108e-3p, cca-miR6108e-5p, cca-miR6108f, cca-miR6108g, cca-miR6109, cca-miR6110-3p, cca-miR6110-5p, cca-miR6111-3p, cca-miR6111-5p, cca-miR6112, cca-miR6113, cca-miR6114-3p, cca-miR6114-5p, cca-miR6115, cca-miR6116-3p, cca-miR6116-5p, cca-miR6117, cca-miR6118-3p, cca-miR6118-5p, ccl-miR167a, ccl-miR167b, ccl-miR168, ccl-miR171, ccl-miR396, ccr-let-7a, ccr-let-7b, ccr-let-7g, ccr-let-7i, ccr-let-7j, ccr-miR-1, ccr-miR-100, ccr-miR-101a, ccr-miR-101b, ccr-miR-103, ccr-miR-107, ccr-miR-10b, ccr-miR-10c, ccr-miR-10d, ccr-miR-122, ccr-miR-124a, ccr-miR-124b, ccr-miR-125b, ccr-miR-125c, ccr-miR-126-3p, ccr-miR-126-5p, ccr-miR-128, ccr-miR-129, ccr-miR-130a, ccr-miR-130b, ccr-miR-130c, ccr-miR-132a, ccr-miR-132b, ccr-miR-133a-3p, ccr-miR-133a-5p, ccr-miR-135c, ccr-miR-137, ccr-miR-138, ccr-miR-139, ccr-miR-140-3p, ccr-miR-140-5p, ccr-miR-142-3p, ccr-miR-142-5p, ccr-miR-143, ccr-miR-144, ccr-miR-146a, ccr-miR-148, ccr-miR-153b, ccr-miR-153c, ccr-miR-155, ccr-miR-15a, ccr-miR-15b, ccr-miR-16a, ccr-miR-16b, ccr-miR-16c, ccr-miR-17-3p, ccr-miR-17-5p, ccr-miR-181a, ccr-miR-181b, ccr-miR-181c, ccr-miR-182-3p, ccr-miR-182-5p, ccr-miR-183, ccr-miR-184, ccr-miR-187, ccr-miR-18a, ccr-miR-18b, ccr-miR-18c, ccr-miR-190, ccr-miR-192, ccr-miR-193a, ccr-miR-194, ccr-miR-196a, ccr-miR-196b, ccr-miR-199-3p, ccr-miR-199-5p, ccr-miR-19d, ccr-miR-200a, ccr-miR-200b, ccr-miR-203a, ccr-miR-203b-3p, ccr-miR-203b-5p, ccr-miR-205, ccr-miR-206, ccr-miR-20a-3p, ccr-miR-20a-5p, ccr-miR-21, ccr-miR-210, ccr-miR-214, ccr-miR-217, ccr-miR-218a, ccr-miR-218b, ccr-miR-2192, ccr-miR-221, ccr-miR-222, ccr-miR-22a, ccr-miR-22b, ccr-miR-23a, ccr-miR-23b, ccr-miR-24, ccr-miR-25, ccr-miR-26a, ccr-miR-27a, ccr-miR-27c-3p, ccr-miR-27c-5p, ccr-miR-27d, ccr-miR-29a, ccr-miR-29b, ccr-miR-301a, ccr-miR-30b, ccr-miR-30d, ccr-miR-338, ccr-miR-34, ccr-miR-363, ccr-miR-365, ccr-miR-375, ccr-miR-429, ccr-miR-430, ccr-miR-454a, ccr-miR-454b, ccr-miR-455, ccr-miR-457a, ccr-miR-457b, ccr-miR-459-3p, ccr-miR-459-5p, ccr-miR-460-3p, ccr-miR-460-5p, ccr-miR-489, ccr-miR-499, ccr-miR-551, ccr-miR-7132, ccr-miR-7133, ccr-miR-722, ccr-miR-724, ccr-miR-725, ccr-miR-726, ccr-miR-727-3p, ccr-miR-727-5p, ccr-miR-729, ccr-miR-730, ccr-miR-734, ccr-miR-738, ccr-miR-7a, ccr-miR-7b, ccr-miR-9-3p, ccr-miR-9-5p, ccr-miR-92a, ccr-miR-92b, ccr-miR-93, ccr-miR-96, ccr-miR-99, cel-let-7-3p, cel-let-7-5p, cel-lin-4-3p, cel-lin-4-5p, cel-lsy-6-3p, cel-lsy-6-5p, cel-miR-1-3p, cel-miR-1-5p, cel-miR-1018, cel-miR-1019-3p, cel-miR-1019-5p, cel-miR-1020-3p, cel-miR-1020-5p, cel-miR-1021, cel-miR-1022-3p, cel-miR-1022-5p, cel-miR-12134, cel-miR-124-3p, cel-miR-124-5p, cel-miR-1817, cel-miR-1818, cel-miR-1819-3p, cel-miR-1819-5p, cel-miR-1820-3p, cel-miR-1820-5p, cel-miR-1821-3p, cel-miR-1821-5p, cel-miR-1822-3p, cel-miR-1822-5p, cel-miR-1823-3p, cel-miR-1823-5p, cel-miR-1824-3p, cel-miR-1824-5p, cel-miR-1828, cel-miR-1829a-3p, cel-miR-1829a-5p, cel-miR-1829b-3p, cel-miR-1829b-5p, cel-miR-1829c-3p, cel-miR-1829c-5p, cel-miR-1830-3p, cel-miR-1830-5p, cel-miR-1832a-3p, cel-miR-1832a-5p, cel-miR-1832b-3p, cel-miR-1832b-5p, cel-miR-1833, cel-miR-2-3p, cel-miR-2-5p, cel-miR-2207-3p, cel-miR-2207-5p, cel-miR-2208a-3p, cel-miR-2208a-5p, cel-miR-2208b-3p, cel-miR-2208b-5p, cel-miR-2209a-3p, cel-miR-2209a-5p, cel-miR-2209b-3p, cel-miR-2209b-5p, cel-miR-2209c-3p, cel-miR-2209c-5p, cel-miR-2210-3p, cel-miR-2210-5p, cel-miR-2211-3p, cel-miR-2211-5p, cel-miR-2212-3p, cel-miR-2212-5p, cel-miR-2213-3p, cel-miR-2213-5p, cel-miR-2215-3p, cel-miR-2215-5p, cel-miR-2216-3p, cel-miR-2216-5p, cel-miR-2217a-3p, cel-miR-2217a-5p, cel-miR-2217b-1-5p, cel-miR-2217b-3p, cel-miR-2217b-5p, cel-miR-2218a-3p, cel-miR-2218a-5p, cel-miR-2218b-3p, cel-miR-2218b-5p, cel-miR-2219-3p, cel-miR-2219-5p, cel-miR-2220-3p, cel-miR-2220-5p, cel-miR-2221, cel-miR-228-3p, cel-miR-228-5p, cel-miR-229-3p, cel-miR-229-5p, cel-miR-230-3p, cel-miR-230-5p, cel-miR-231-3p, cel-miR-231-5p, cel-miR-232-3p, cel-miR-232-5p, cel-miR-233-3p, cel-miR-233-5p, cel-miR-234-3p, cel-miR-234-5p, cel-miR-235-3p, cel-miR-235-5p, cel-miR-236-3p, cel-miR-236-5p, cel-miR-237-3p, cel-miR-237-5p, cel-miR-238-3p, cel-miR-238-5p, cel-miR-239a-3p, cel-miR-239a-5p, cel-miR-239b-3p, cel-miR-239b-5p, cel-miR-240-3p, cel-miR-240-5p, cel-miR-241-3p, cel-miR-241-5p, cel-miR-242, cel-miR-243-3p, cel-miR-243-5p, cel-miR-244-3p, cel-miR-244-5p, cel-miR-245-3p, cel-miR-245-5p, cel-miR-246-3p, cel-miR-246-5p, cel-miR-247-3p, cel-miR-247-5p, cel-miR-248, cel-miR-249-3p, cel-miR-249-5p, cel-miR-250-3p, cel-miR-250-5p, cel-miR-251, cel-miR-252-3p, cel-miR-252-5p, cel-miR-253-3p, cel-miR-253-5p, cel-miR-254-3p, cel-miR-254-5p, cel-miR-255-3p, cel-miR-255-5p, cel-miR-256, cel-miR-259-3p, cel-miR-259-5p, cel-miR-261, cel-miR-264, cel-miR-265, cel-miR-266, cel-miR-267, cel-miR-268, cel-miR-269, cel-miR-270, cel-miR-271, cel-miR-272, cel-miR-273, cel-miR-2953-3p, cel-miR-2953-5p, cel-miR-34-3p, cel-miR-34-5p, cel-miR-35-3p, cel-miR-35-5p, cel-miR-354-3p, cel-miR-354-5p, cel-miR-355-3p, cel-miR-355-5p, cel-miR-356a, cel-miR-356b-3p, cel-miR-356b-5p, cel-miR-357-3p, cel-miR-357-5p, cel-miR-358-3p, cel-miR-358-5p, cel-miR-359, cel-miR-36-3p, cel-miR-36-5p, cel-miR-360-3p, cel-miR-360-5p, cel-miR-37-3p, cel-miR-37-5p, cel-miR-38-3p, cel-miR-38-5p, cel-miR-39-3p, cel-miR-39-5p, cel-miR-392-3p, cel-miR-392-5p, cel-miR-40-3p, cel-miR-40-5p, cel-miR-41-3p, cel-miR-41-5p, cel-miR-42-3p, cel-miR-42-5p, cel-miR-43-3p, cel-miR-43-5p, cel-miR-44-3p, cel-miR-44-5p, cel-miR-45-3p, cel-miR-45-5p, cel-miR-46-3p, cel-miR-46-5p, cel-miR-47-3p, cel-miR-47-5p, cel-miR-48-3p, cel-miR-48-5p, cel-miR-4805-3p, cel-miR-4805-5p, cel-miR-4806-3p, cel-miR-4806-5p, cel-miR-4807, cel-miR-4808-3p, cel-miR-4808-5p, cel-miR-4809-3p, cel-miR-4809-5p, cel-miR-4810a, cel-miR-4810b-3p, cel-miR-4810b-5p, cel-miR-4811-3p, cel-miR-4811-5p, cel-miR-4812-3p, cel-miR-4812-5p, cel-miR-4813-3p, cel-miR-4813-5p, cel-miR-4814-3p, cel-miR-4814-5p, cel-miR-4815, cel-miR-4816-3p, cel-miR-4816-5p, cel-miR-49-3p, cel-miR-49-5p, cel-miR-4920, cel-miR-4921, cel-miR-4922, cel-miR-4923a, cel-miR-4923b, cel-miR-4924, cel-miR-4925, cel-miR-4926, cel-miR-4927, cel-miR-4929, cel-miR-4930, cel-miR-4931, cel-miR-4932, cel-miR-4933, cel-miR-4934, cel-miR-4935, cel-miR-4936, cel-miR-4937, cel-miR-4938, cel-miR-50-3p, cel-miR-50-5p, cel-miR-51-3p, cel-miR-51-5p, cel-miR-52-3p, cel-miR-52-5p, cel-miR-53-3p, cel-miR-53-5p, cel-miR-54-3p, cel-miR-54-5p, cel-miR-55-3p, cel-miR-55-5p, cel-miR-5545-3p, cel-miR-5545-5p, cel-miR-5546-3p, cel-miR-5546-5p, cel-miR-5547-3p, cel-miR-5547-5p, cel-miR-5548-3p, cel-miR-5548-5p, cel-miR-5549-3p, cel-miR-5549-5p, cel-miR-5550-3p, cel-miR-5550-5p, cel-miR-5551-3p, cel-miR-5551-5p, cel-miR-5552-3p, cel-miR-5552-5p, cel-miR-5553-3p, cel-miR-5553-5p, cel-miR-5592-3p, cel-miR-5592-5p, cel-miR-5593-3p, cel-miR-5593-5p, cel-miR-5594-3p, cel-miR-5594-5p, cel-miR-5595-3p, cel-miR-5595-5p, cel-miR-55b, cel-miR-56-3p, cel-miR-56-5p, cel-miR-57-3p, cel-miR-57-5p, cel-miR-58a-3p, cel-miR-58a-5p, cel-miR-58b-3p, cel-miR-58b-5p, cel-miR-58c, cel-miR-59-3p, cel-miR-59-5p, cel-miR-60-3p, cel-miR-60-5p, cel-miR-61-3p, cel-miR-61-5p, cel-miR-62, cel-miR-63-3p, cel-miR-63-5p, cel-miR-64-3p, cel-miR-64-5p, cel-miR-65-3p, cel-miR-65-5p, cel-miR-66-3p, cel-miR-66-5p, cel-miR-67-3p, cel-miR-67-5p, cel-miR-70-3p, cel-miR-70-5p, cel-miR-71-3p, cel-miR-71-5p, cel-miR-72-3p, cel-miR-72-5p, cel-miR-73-3p, cel-miR-73-5p, cel-miR-74-3p, cel-miR-74-5p, cel-miR-75-3p, cel-miR-75-5p, cel-miR-76-3p, cel-miR-76-5p, cel-miR-77-3p, cel-miR-77-5p, cel-miR-78, cel-miR-784-3p, cel-miR-784-5p, cel-miR-785-3p, cel-miR-785-5p, cel-miR-786-3p, cel-miR-786-5p, cel-miR-787-3p, cel-miR-787-5p, cel-miR-788-3p, cel-miR-788-5p, cel-miR-789-2-5p, cel-miR-789-3p, cel-miR-789-5p, cel-miR-79-3p, cel-miR-79-5p, cel-miR-790-3p, cel-miR-790-5p, cel-miR-791-3p, cel-miR-791-5p, cel-miR-792-3p, cel-miR-792-5p, cel-miR-793, cel-miR-794-3p, cel-miR-794-5p, cel-miR-795-3p, cel-miR-795-5p, cel-miR-796, cel-miR-797-3p, cel-miR-797-5p, cel-miR-798, cel-miR-799, cel-miR-80-3p, cel-miR-80-5p, cel-miR-800-3p, cel-miR-800-5p, cel-miR-81-3p, cel-miR-81-5p, cel-miR-8186-3p, cel-miR-8186-5p, cel-miR-8187-3p, cel-miR-8187-5p, cel-miR-8188-3p, cel-miR-8188-5p, cel-miR-8189-3p, cel-miR-8189-5p, cel-miR-8190-3p, cel-miR-8190-5p, cel-miR-8191-3p, cel-miR-8191-5p, cel-miR-8192-3p, cel-miR-8192-5p, cel-miR-8193-3p, cel-miR-8193-5p, cel-miR-8194-3p, cel-miR-8194-5p, cel-miR-8195-3p, cel-miR-8195-5p, cel-miR-8196a-3p, cel-miR-8196a-5p, cel-miR-8196b-3p, cel-miR-8196b-5p, cel-miR-8197-3p, cel-miR-8197-5p, cel-miR-8198-3p, cel-miR-8198-5p, cel-miR-8199-3p, cel-miR-8199-5p, cel-miR-82-3p, cel-miR-82-5p, cel-miR-8200-3p, cel-miR-8200-5p, cel-miR-8201-3p, cel-miR-8201-5p, cel-miR-8202-3p, cel-miR-8202-5p, cel-miR-8203-3p, cel-miR-8203-5p, cel-miR-8204-3p, cel-miR-8204-5p, cel-miR-8205-3p, cel-miR-8205-5p, cel-miR-8206-3p, cel-miR-8206-5p, cel-miR-8207-3p, cel-miR-8207-5p, cel-miR-8208-3p, cel-miR-8208-5p, cel-miR-8209-3p, cel-miR-8209-5p, cel-miR-8210-3p, cel-miR-8210-5p, cel-miR-8211-3p, cel-miR-8211-5p, cel-miR-8212-3p, cel-miR-8212-5p, cel-miR-83-3p, cel-miR-83-5p, cel-miR-84-3p, cel-miR-84-5p, cel-miR-85-3p, cel-miR-85-5p, cel-miR-86-3p, cel-miR-86-5p, cel-miR-87-3p, cel-miR-87-5p, cel-miR-90-3p, cel-miR-90-5p, cfa-let-7a, cfa-let-7b, cfa-let-7c, cfa-let-7d, cfa-let-7e, cfa-let-7f, cfa-let-7g, cfa-let-7j, cfa-miR-1, cfa-miR-101, cfa-miR-103, cfa-miR-105a, cfa-miR-105b, cfa-miR-106a, cfa-miR-106b, cfa-miR-107, cfa-miR-10a, cfa-miR-10b, cfa-miR-1185, cfa-miR-1199-3p, cfa-miR-1199-5p, cfa-miR-122, cfa-miR-124, cfa-miR-1249, cfa-miR-125a, cfa-miR-125b, cfa-miR-126, cfa-miR-127, cfa-miR-1271, cfa-miR-128, cfa-miR-129, cfa-miR-1296, cfa-miR-1301, cfa-miR-1306, cfa-miR-1307, cfa-miR-130a, cfa-miR-130b, cfa-miR-132, cfa-miR-133a, cfa-miR-133b, cfa-miR-133c, cfa-miR-134, cfa-miR-1343, cfa-miR-135a-3p, cfa-miR-135a-5p, cfa-miR-135b, cfa-miR-136, cfa-miR-137, cfa-miR-138a, cfa-miR-138b, cfa-miR-139, cfa-miR-140, cfa-miR-141, cfa-miR-142, cfa-miR-143, cfa-miR-144, cfa-miR-145, cfa-miR-1468, cfa-miR-146a, cfa-miR-146b, cfa-miR-147, cfa-miR-148a, cfa-miR-148b, cfa-miR-149, cfa-miR-150, cfa-miR-151, cfa-miR-152, cfa-miR-153, cfa-miR-155, cfa-miR-15a, cfa-miR-15b, cfa-miR-16, cfa-miR-17, cfa-miR-181a, cfa-miR-181b, cfa-miR-181c, cfa-miR-181d, cfa-miR-182, cfa-miR-183, cfa-miR-1835, cfa-miR-1836, cfa-miR-1837, cfa-miR-1838, cfa-miR-1839, cfa-miR-184, cfa-miR-1840, cfa-miR-1841, cfa-miR-1842, cfa-miR-1843, cfa-miR-1844, cfa-miR-185, cfa-miR-186, cfa-miR-187, cfa-miR-188, cfa-miR-18a, cfa-miR-18b, cfa-miR-190a, cfa-miR-190b, cfa-miR-191, cfa-miR-192, cfa-miR-193a, cfa-miR-193b, cfa-miR-194, cfa-miR-195, cfa-miR-196a, cfa-miR-196b, cfa-miR-197, cfa-miR-199, cfa-miR-19a, cfa-miR-19b, cfa-miR-200a, cfa-miR-200b, cfa-miR-200c, cfa-miR-202, cfa-miR-203, cfa-miR-204, cfa-miR-205, cfa-miR-206, cfa-miR-207, cfa-miR-208a, cfa-miR-208b, cfa-miR-20a, cfa-miR-20b, cfa-miR-21, cfa-miR-210, cfa-miR-211, cfa-miR-2114, cfa-miR-212, cfa-miR-214, cfa-miR-215, cfa-miR-216a, cfa-miR-216b, cfa-miR-217, cfa-miR-218, cfa-miR-219-3p, cfa-miR-219-5p, cfa-miR-22, cfa-miR-221, cfa-miR-222, cfa-miR-223, cfa-miR-224, cfa-miR-2387, cfa-miR-23a, cfa-miR-23b, cfa-miR-24, cfa-miR-2483, cfa-miR-25, cfa-miR-26a, cfa-miR-26b, cfa-miR-27a, cfa-miR-27b, cfa-miR-28, cfa-miR-299, cfa-miR-29a, cfa-miR-29b, cfa-miR-29c, cfa-miR-300, cfa-miR-301a, cfa-miR-301b, cfa-miR-302a, cfa-miR-302b, cfa-miR-302c, cfa-miR-302d, cfa-miR-30a, cfa-miR-30b, cfa-miR-30c, cfa-miR-30d, cfa-miR-30e, cfa-miR-31, cfa-miR-32, cfa-miR-320, cfa-miR-323, cfa-miR-324, cfa-miR-325, cfa-miR-326, cfa-miR-328, cfa-miR-329a, cfa-miR-329b, cfa-miR-330, cfa-miR-331, cfa-miR-335, cfa-miR-338, cfa-miR-339, cfa-miR-33a, cfa-miR-33b, cfa-miR-340, cfa-miR-342, cfa-miR-345, cfa-miR-346, cfa-miR-34a, cfa-miR-34b, cfa-miR-34c, cfa-miR-350, cfa-miR-361, cfa-miR-362, cfa-miR-363, cfa-miR-365, cfa-miR-367, cfa-miR-369, cfa-miR-370, cfa-miR-371, cfa-miR-374a, cfa-miR-374b, cfa-miR-375, cfa-miR-376a, cfa-miR-376b, cfa-miR-376c, cfa-miR-377, cfa-miR-378, cfa-miR-379, cfa-miR-380, cfa-miR-381, cfa-miR-382, cfa-miR-383, cfa-miR-384, cfa-miR-3958, cfa-miR-409, cfa-miR-410, cfa-miR-411, cfa-miR-421, cfa-miR-423a, cfa-miR-424, cfa-miR-425, cfa-miR-429, cfa-miR-432, cfa-miR-433, cfa-miR-448, cfa-miR-449a, cfa-miR-449b, cfa-miR-450a, cfa-miR-450b, cfa-miR-451, cfa-miR-452, cfa-miR-454, cfa-miR-455, cfa-miR-483, cfa-miR-485, cfa-miR-486, cfa-miR-486-3p, cfa-miR-487a, cfa-miR-487b, cfa-miR-488, cfa-miR-489, cfa-miR-490, cfa-miR-491, cfa-miR-493, cfa-miR-494, cfa-miR-495, cfa-miR-496, cfa-miR-497, cfa-miR-499, cfa-miR-500, cfa-miR-502, cfa-miR-503, cfa-miR-504, cfa-miR-505, cfa-miR-506, cfa-miR-507a, cfa-miR-507b, cfa-miR-508a, cfa-miR-508b, cfa-miR-514, cfa-miR-532, cfa-miR-539, cfa-miR-542, cfa-miR-543, cfa-miR-544, cfa-miR-545, cfa-miR-551a, cfa-miR-551b, cfa-miR-568, cfa-miR-574, cfa-miR-578, cfa-miR-582, cfa-miR-589, cfa-miR-590, cfa-miR-592, cfa-miR-599, cfa-miR-615, cfa-miR-628, cfa-miR-631, cfa-miR-632, cfa-miR-6516, cfa-miR-652, cfa-miR-6529, cfa-miR-653, cfa-miR-660, cfa-miR-664, cfa-miR-665, cfa-miR-671, cfa-miR-676, cfa-miR-7, cfa-miR-708, cfa-miR-718, cfa-miR-7180, cfa-miR-758, cfa-miR-759, cfa-miR-761, cfa-miR-764, cfa-miR-769, cfa-miR-802, cfa-miR-872, cfa-miR-874, cfa-miR-875, cfa-miR-876, cfa-miR-8789, cfa-miR-8790, cfa-miR-8791a, cfa-miR-8791b, cfa-miR-8792, cfa-miR-8793, cfa-miR-8794, cfa-miR-8795, cfa-miR-8796, cfa-miR-8797, cfa-miR-8798, cfa-miR-8799a, cfa-miR-8799b, cfa-miR-8799c, cfa-miR-8799d, cfa-miR-8799e, cfa-miR-8799f, cfa-miR-8799g, cfa-miR-8800, cfa-miR-8801, cfa-miR-8802, cfa-miR-8803, cfa-miR-8804, cfa-miR-8805, cfa-miR-8806, cfa-miR-8807, cfa-miR-8808, cfa-miR-8809, cfa-miR-8810, cfa-miR-8811, cfa-miR-8812, cfa-miR-8813, cfa-miR-8814, cfa-miR-8815, cfa-miR-8816, cfa-miR-8817, cfa-miR-8818, cfa-miR-8819, cfa-miR-8820, cfa-miR-8821, cfa-miR-8822, cfa-miR-8823, cfa-miR-8824, cfa-miR-8825, cfa-miR-8826, cfa-miR-8827, cfa-miR-8828, cfa-miR-8829, cfa-miR-8830, cfa-miR-8831, cfa-miR-8832, cfa-miR-8833, cfa-miR-8834a, cfa-miR-8834b, cfa-miR-8835, cfa-miR-8836, cfa-miR-8837, cfa-miR-8838, cfa-miR-8839, cfa-miR-8840, cfa-miR-8841, cfa-miR-8842, cfa-miR-8843, cfa-miR-8844, cfa-miR-8845, cfa-miR-8846, cfa-miR-8847, cfa-miR-8848, cfa-miR-8849, cfa-miR-885, cfa-miR-8850, cfa-miR-8851, cfa-miR-8852, cfa-miR-8853, cfa-miR-8854, cfa-miR-8855, cfa-miR-8856, cfa-miR-8857, cfa-miR-8858, cfa-miR-8859a, cfa-miR-8859b, cfa-miR-8860, cfa-miR-8861, cfa-miR-8862, cfa-miR-8863, cfa-miR-8864, cfa-miR-8865, cfa-miR-8866, cfa-miR-8867, cfa-miR-8868, cfa-miR-8869, cfa-miR-8870, cfa-miR-8871, cfa-miR-8872, cfa-miR-8873a, cfa-miR-8873b, cfa-miR-8874, cfa-miR-8875, cfa-miR-8876, cfa-miR-8877, cfa-miR-8878, cfa-miR-8879, cfa-miR-8880, cfa-miR-8881, cfa-miR-8882, cfa-miR-8883, cfa-miR-8884, cfa-miR-8885, cfa-miR-8886, cfa-miR-8887, cfa-miR-8888, cfa-miR-8889, cfa-miR-889, cfa-miR-8890, cfa-miR-8891, cfa-miR-8892, cfa-miR-8893, cfa-miR-8894, cfa-miR-8895, cfa-miR-8896, cfa-miR-8897, cfa-miR-8898, cfa-miR-8899, cfa-miR-8900, cfa-miR-8901, cfa-miR-8902, cfa-miR-8903, cfa-miR-8904a, cfa-miR-8904b, cfa-miR-8905, cfa-miR-8906, cfa-miR-8907, cfa-miR-8908a-3p, cfa-miR-8908a-5p, cfa-miR-8908b, cfa-miR-8908c, cfa-miR-8908d, cfa-miR-8908e, cfa-miR-8908f, cfa-miR-9, cfa-miR-92a, cfa-miR-92b, cfa-miR-93, cfa-miR-95, cfa-miR-96, cfa-miR-98, cfa-miR-99a, cfa-miR-99b, cgr-let-7a, cgr-let-7a-2, cgr-let-7b, cgr-let-7c, cgr-let-7c-2, cgr-let-7d-3p, cgr-let-7d-5p, cgr-let-7e, cgr-let-7f, cgr-let-7g-3p, cgr-let-7g-5p, cgr-let-7i, cgr-miR-1, cgr-miR-100-3p, cgr-miR-100-5p, cgr-miR-101a, cgr-miR-101b-3p, cgr-miR-101b-5p, cgr-miR-103-3p, cgr-miR-103-5p, cgr-miR-106b-3p, cgr-miR-106b-5p, cgr-miR-107, cgr-miR-10a-3p, cgr-miR-10a-5p, cgr-miR-10b-3p, cgr-miR-10b-5p, cgr-miR-122, cgr-miR-1224, cgr-miR-124, cgr-miR-1249, cgr-miR-125a-3p, cgr-miR-125a-5p, cgr-miR-125b, cgr-miR-125b-3p, cgr-miR-125b-5p, cgr-miR-1260, cgr-miR-126a, cgr-miR-126b, cgr-miR-127, cgr-miR-1271, cgr-miR-128, cgr-miR-128-3p, cgr-miR-128-5p, cgr-miR-1285, cgr-miR-129, cgr-miR-1306-3p, cgr-miR-1306-5p, cgr-miR-130a-3p, cgr-miR-130a-5p, cgr-miR-130b-3p, cgr-miR-130b-5p, cgr-miR-132-3p, cgr-miR-132-5p, cgr-miR-134, cgr-miR-1343, cgr-miR-137-3p, cgr-miR-137-5p, cgr-miR-138, cgr-miR-139-3p, cgr-miR-139-5p, cgr-miR-140-3p, cgr-miR-140-5p, cgr-miR-141, cgr-miR-142-3p, cgr-miR-142-5p, cgr-miR-143, cgr-miR-144, cgr-miR-146a, cgr-miR-146b-3p, cgr-miR-146b-5p, cgr-miR-147, cgr-miR-148a, cgr-miR-148b-3p, cgr-miR-148b-5p, cgr-miR-149-3p, cgr-miR-149-5p, cgr-miR-150, cgr-miR-151-3p, cgr-miR-151-5p, cgr-miR-152-3p, cgr-miR-152-5p, cgr-miR-154-3p, cgr-miR-154-5p, cgr-miR-155, cgr-miR-15a-3p, cgr-miR-15a-5p, cgr-miR-15b-3p, cgr-miR-15b-5p, cgr-miR-16, cgr-miR-16-3p, cgr-miR-16-5p, cgr-miR-17-3p, cgr-miR-17-5p, cgr-miR-181a-3p, cgr-miR-181a-5p, cgr-miR-181b-3p, cgr-miR-181b-5p, cgr-miR-181c-3p, cgr-miR-181c-5p, cgr-miR-181d-3p, cgr-miR-181d-5p, cgr-miR-182, cgr-miR-183, cgr-miR-1839-3p, cgr-miR-1839-5p, cgr-miR-184, cgr-miR-1843, cgr-miR-185-3p, cgr-miR-185-5p, cgr-miR-186-3p, cgr-miR-186-5p, cgr-miR-187, cgr-miR-188, cgr-miR-18a-3p, cgr-miR-18a-5p, cgr-miR-18b, cgr-miR-1903, cgr-miR-190a, cgr-miR-190b, cgr-miR-191-3p, cgr-miR-191-5p, cgr-miR-192, cgr-miR-193a, cgr-miR-193b-3p, cgr-miR-193b-5p, cgr-miR-194, cgr-miR-194-2, cgr-miR-1949, cgr-miR-195, cgr-miR-1956, cgr-miR-196a-3p, cgr-miR-196a-5p, cgr-miR-196b, cgr-miR-196c, cgr-miR-1973, cgr-miR-199a, cgr-miR-199a-3p, cgr-miR-199a-5p, cgr-miR-199b, cgr-miR-19a, cgr-miR-19b-3p, cgr-miR-19b-5p, cgr-miR-1b, cgr-miR-200a, cgr-miR-200b, cgr-miR-200c, cgr-miR-202, cgr-miR-204, cgr-miR-205, cgr-miR-206, cgr-miR-20a, cgr-miR-20b, cgr-miR-21-3p, cgr-miR-21-5p, cgr-miR-210-3p, cgr-miR-210-5p, cgr-miR-211, cgr-miR-212, cgr-miR-214-3p, cgr-miR-214-5p, cgr-miR-215-3p, cgr-miR-215-5p, cgr-miR-216b, cgr-miR-217, cgr-miR-218a, cgr-miR-219b, cgr-miR-22-3p, cgr-miR-22-5p, cgr-miR-221-3p, cgr-miR-221-5p, cgr-miR-222-3p, cgr-miR-222-5p, cgr-miR-23a-3p, cgr-miR-23a-5p, cgr-miR-23b-3p, cgr-miR-23b-5p, cgr-miR-24, cgr-miR-24-3p, cgr-miR-24-5p, cgr-miR-2424, cgr-miR-25-3p, cgr-miR-25-5p, cgr-miR-26a, cgr-miR-26a-2, cgr-miR-26b-3p, cgr-miR-26b-5p, cgr-miR-27a-3p, cgr-miR-27a-5p, cgr-miR-27b-3p, cgr-miR-27b-5p, cgr-miR-28-3p, cgr-miR-28-5p, cgr-miR-296, cgr-miR-298-3p, cgr-miR-298-5p, cgr-miR-29a-3p, cgr-miR-29a-5p, cgr-miR-29b, cgr-miR-29b-3p, cgr-miR-29b-5p, cgr-miR-29c-3p, cgr-miR-29c-5p, cgr-miR-300, cgr-miR-301a-3p, cgr-miR-301a-5p, cgr-miR-301b, cgr-miR-3068, cgr-miR-3072-3p, cgr-miR-3072-5p, cgr-miR-3074, cgr-miR-30a-3p, cgr-miR-30a-5p, cgr-miR-30b-3p, cgr-miR-30b-5p, cgr-miR-30c, cgr-miR-30c-2, cgr-miR-30d, cgr-miR-30e-3p, cgr-miR-30e-5p, cgr-miR-31-3p, cgr-miR-31-5p, cgr-miR-3102, cgr-miR-3154, cgr-miR-32-3p, cgr-miR-32-5p, cgr-miR-320a, cgr-miR-322-3p, cgr-miR-322-5p, cgr-miR-324-3p, cgr-miR-324-5p, cgr-miR-325-3p, cgr-miR-325-5p, cgr-miR-326, cgr-miR-328, cgr-miR-33, cgr-miR-330, cgr-miR-331-3p, cgr-miR-331-5p, cgr-miR-338, cgr-miR-339, cgr-miR-340-3p, cgr-miR-340-5p, cgr-miR-342-3p, cgr-miR-342-5p, cgr-miR-344, cgr-miR-345, cgr-miR-34a, cgr-miR-34b-3p, cgr-miR-34b-5p, cgr-miR-34c-3p, cgr-miR-34c-5p, cgr-miR-350-3p, cgr-miR-350-5p, cgr-miR-3535, cgr-miR-361, cgr-miR-362, cgr-miR-365, cgr-miR-365-3p, cgr-miR-365-5p, cgr-miR-369-3p, cgr-miR-369-5p, cgr-miR-374-3p, cgr-miR-374-5p, cgr-miR-377-3p, cgr-miR-377-5p, cgr-miR-378-3p, cgr-miR-378-5p, cgr-miR-379, cgr-miR-381, cgr-miR-382, cgr-miR-384-3p, cgr-miR-384-5p, cgr-miR-409-3p, cgr-miR-409-5p, cgr-miR-410-3p, cgr-miR-410-5p, cgr-miR-412-3p, cgr-miR-412-5p, cgr-miR-423-3p, cgr-miR-423-5p, cgr-miR-425-3p, cgr-miR-425-5p, cgr-miR-429, cgr-miR-449a, cgr-miR-450a, cgr-miR-450b-3p, cgr-miR-450b-5p, cgr-miR-451a, cgr-miR-455-3p, cgr-miR-455-5p, cgr-miR-484, cgr-miR-486-3p, cgr-miR-486-5p, cgr-miR-496, cgr-miR-497-3p, cgr-miR-497-5p, cgr-miR-497b, cgr-miR-499-3p, cgr-miR-499-5p, cgr-miR-500, cgr-miR-501-3p, cgr-miR-501-5p, cgr-miR-503, cgr-miR-504, cgr-miR-505-3p, cgr-miR-505-5p, cgr-miR-532-3p, cgr-miR-532-5p, cgr-miR-542-3p, cgr-miR-542-5p, cgr-miR-574, cgr-miR-582, cgr-miR-598, cgr-miR-6091, cgr-miR-6092, cgr-miR-615-3p, cgr-miR-615-5p, cgr-miR-628, cgr-miR-632, cgr-miR-652-3p, cgr-miR-652-5p, cgr-miR-653, cgr-miR-664-3p, cgr-miR-664-5p, cgr-miR-671-3p, cgr-miR-671-5p, cgr-miR-672, cgr-miR-674, cgr-miR-702, cgr-miR-708, cgr-miR-744-3p, cgr-miR-744-5p, cgr-miR-760, cgr-miR-7a, cgr-miR-7b, cgr-miR-802, cgr-miR-872-3p, cgr-miR-872-5p, cgr-miR-874, cgr-miR-9, cgr-miR-92a-3p, cgr-miR-92a-5p, cgr-miR-92b-3p, cgr-miR-92b-5p, cgr-miR-93-3p, cgr-miR-93-5p, cgr-miR-98, cgr-miR-99a-3p, cgr-miR-99a-5p, cgr-miR-99b, chi-let-7a-3p, chi-let-7a-5p, chi-let-7b-3p, chi-let-7b-5p, chi-let-7c-3p, chi-let-7c-5p, chi-let-7d-3p, chi-let-7d-5p, chi-let-7e-3p, chi-let-7e-5p, chi-let-7f-3p, chi-let-7f-5p, chi-let-7g-3p, chi-let-7g-5p, chi-let-7i-3p, chi-let-7i-5p, chi-miR-1, chi-miR-100-3p, chi-miR-100-5p, chi-miR-101-3p, chi-miR-101-5p, chi-miR-103-3p, chi-miR-103-5p, chi-miR-105a, chi-miR-105b-3p, chi-miR-105b-5p, chi-miR-106a-3p, chi-miR-106a-5p, chi-miR-106b-3p, chi-miR-106b-5p, chi-miR-107-3p, chi-miR-10a-3p, chi-miR-10a-5p, chi-miR-10b-3p, chi-miR-10b-5p, chi-miR-1185-3p, chi-miR-1185-5p, chi-miR-1197-3p, chi-miR-122, chi-miR-1224, chi-miR-1248-5p, chi-miR-1249, chi-miR-124a, chi-miR-125a-3p, chi-miR-125a-5p, chi-miR-125b-3p, chi-miR-125b-5p, chi-miR-126-3p, chi-miR-126-5p, chi-miR-127-3p, chi-miR-127-5p, chi-miR-1271-3p, chi-miR-1271-5p, chi-miR-128-3p, chi-miR-128-5p, chi-miR-129-3p, chi-miR-129-5p, chi-miR-1291, chi-miR-1296, chi-miR-1306-3p, chi-miR-1306-5p, chi-miR-1307-3p, chi-miR-1307-5p, chi-miR-130a-3p, chi-miR-130a-5p, chi-miR-130b-3p, chi-miR-130b-5p, chi-miR-133a-3p, chi-miR-133a-5p, chi-miR-133b, chi-miR-134, chi-miR-1343, chi-miR-135a, chi-miR-135b-3p, chi-miR-135b-5p, chi-miR-136-3p, chi-miR-136-5p, chi-miR-137, chi-miR-1388-3p, chi-miR-1388-5p, chi-miR-140-3p, chi-miR-140-5p, chi-miR-141, chi-miR-143-3p, chi-miR-143-5p, chi-miR-144-3p, chi-miR-144-5p, chi-miR-145-3p, chi-miR-145-5p, chi-miR-1468-3p, chi-miR-1468-5p, chi-miR-146a, chi-miR-146b-3p, chi-miR-146b-5p, chi-miR-147-3p, chi-miR-147-5p, chi-miR-148a-3p, chi-miR-148a-5p, chi-miR-148b-3p, chi-miR-148b-5p, chi-miR-150, chi-miR-151-3p, chi-miR-151-5p, chi-miR-153, chi-miR-154a-3p, chi-miR-154a-5p, chi-miR-154b-3p, chi-miR-154b-5p, chi-miR-155-3p, chi-miR-155-5p, chi-miR-15a-3p, chi-miR-15a-5p, chi-miR-15b-3p, chi-miR-15b-5p, chi-miR-16a-3p, chi-miR-16a-5p, chi-miR-16b-3p, chi-miR-16b-5p, chi-miR-17-3p, chi-miR-17-5p, chi-miR-1814, chi-miR-181b-3p, chi-miR-181b-5p, chi-miR-181c-3p, chi-miR-181c-5p, chi-miR-181d, chi-miR-182, chi-miR-183, chi-miR-1839, chi-miR-184, chi-miR-186-3p, chi-miR-186-5p, chi-miR-187, chi-miR-188-3p, chi-miR-188-5p, chi-miR-18a-3p, chi-miR-18a-5p, chi-miR-18b-3p, chi-miR-18b-5p, chi-miR-190a-3p, chi-miR-190a-5p, chi-miR-190b, chi-miR-191-3p, chi-miR-191-5p, chi-miR-192-3p, chi-miR-192-5p, chi-miR-193a, chi-miR-193b-3p, chi-miR-193b-5p, chi-miR-194, chi-miR-195-3p, chi-miR-195-5p, chi-miR-196a, chi-miR-196b, chi-miR-197-3p, chi-miR-197-5p, chi-miR-199a-3p, chi-miR-199a-5p, chi-miR-199b-3p, chi-miR-199b-5p, chi-miR-199c-3p, chi-miR-199c-5p, chi-miR-19a, chi-miR-19b-3p, chi-miR-19b-5p, chi-miR-200a, chi-miR-200b, chi-miR-200c, chi-miR-202-3p, chi-miR-202-5p, chi-miR-204-3p, chi-miR-204-5p, chi-miR-206, chi-miR-208b, chi-miR-20a-3p, chi-miR-20a-5p, chi-miR-20b, chi-miR-21-3p, chi-miR-21-5p, chi-miR-211, chi-miR-214-3p, chi-miR-214-5p, chi-miR-215-5p, chi-miR-216b, chi-miR-217-3p, chi-miR-217-5p, chi-miR-218, chi-miR-219, chi-miR-22-3p, chi-miR-22-5p, chi-miR-221-3p, chi-miR-221-5p, chi-miR-222-3p, chi-miR-223-3p, chi-miR-223-5p, chi-miR-224-3p, chi-miR-224-5p, chi-miR-2284a, chi-miR-2284b, chi-miR-2284c, chi-miR-2284d, chi-miR-2284e, chi-miR-2290, chi-miR-2318, chi-miR-2320, chi-miR-2331, chi-miR-2332, chi-miR-2335, chi-miR-23a, chi-miR-23b-3p, chi-miR-23b-5p, chi-miR-24-3p, chi-miR-24-5p, chi-miR-2404, chi-miR-2411-3p, chi-miR-2411-5p, chi-miR-2432, chi-miR-2483-3p, chi-miR-2483-5p, chi-miR-25-3p, chi-miR-25-5p, chi-miR-26a-3p, chi-miR-26a-5p, chi-miR-26b-3p, chi-miR-26b-5p, chi-miR-27a-3p, chi-miR-27a-5p, chi-miR-27b-3p, chi-miR-27b-5p, chi-miR-28-3p, chi-miR-28-5p, chi-miR-296-3p, chi-miR-29a-3p, chi-miR-29a-5p, chi-miR-29b-3p, chi-miR-29b-5p, chi-miR-29c-3p, chi-miR-29c-5p, chi-miR-301a-3p, chi-miR-301a-5p, chi-miR-301b, chi-miR-30a-3p, chi-miR-30a-5p, chi-miR-30b-3p, chi-miR-30b-5p, chi-miR-30c-3p, chi-miR-30c-5p, chi-miR-30d-3p, chi-miR-30e-3p, chi-miR-30e-5p, chi-miR-30f-3p, chi-miR-30f-5p, chi-miR-320-3p, chi-miR-323a-3p, chi-miR-323b, chi-miR-324-3p, chi-miR-324-5p, chi-miR-326-3p, chi-miR-326-5p, chi-miR-328-3p, chi-miR-329a-3p, chi-miR-329a-5p, chi-miR-329b-3p, chi-miR-330-3p, chi-miR-330-5p, chi-miR-331-3p, chi-miR-331-5p, chi-miR-335-3p, chi-miR-335-5p, chi-miR-338-3p, chi-miR-338-5p, chi-miR-33a-3p, chi-miR-33a-5p, chi-miR-33b-3p, chi-miR-33b-5p, chi-miR-340-3p, chi-miR-340-5p, chi-miR-342-3p, chi-miR-342-5p, chi-miR-3431-3p, chi-miR-3431-5p, chi-miR-3432-3p, chi-miR-3432-5p, chi-miR-345-3p, chi-miR-345-5p, chi-miR-346-5p, chi-miR-34a, chi-miR-34b-3p, chi-miR-34b-5p, chi-miR-34c-3p, chi-miR-34c-5p, chi-miR-361-3p, chi-miR-361-5p, chi-miR-362-3p, chi-miR-362-5p, chi-miR-363-3p, chi-miR-363-5p, chi-miR-365-3p, chi-miR-369-3p, chi-miR-369-5p, chi-miR-374a-3p, chi-miR-374a-5p, chi-miR-374b-3p, chi-miR-374b-5p, chi-miR-376a, chi-miR-376b-3p, chi-miR-376b-5p, chi-miR-376c-3p, chi-miR-376c-5p, chi-miR-376d, chi-miR-376e-3p, chi-miR-376e-5p, chi-miR-377, chi-miR-378-3p, chi-miR-378-5p, chi-miR-379-3p, chi-miR-379-5p, chi-miR-380-3p, chi-miR-380-5p, chi-miR-381, chi-miR-382-3p, chi-miR-382-5p, chi-miR-383, chi-miR-3955-3p, chi-miR-3955-5p, chi-miR-3958-3p, chi-miR-3958-5p, chi-miR-3959-3p, chi-miR-3959-5p, chi-miR-409-3p, chi-miR-409-5p, chi-miR-410-3p, chi-miR-410-5p, chi-miR-411a-3p, chi-miR-411a-5p, chi-miR-411b-3p, chi-miR-411b-5p, chi-miR-412-3p, chi-miR-412-5p, chi-miR-421-3p, chi-miR-423-3p, chi-miR-423-5p, chi-miR-424-3p, chi-miR-424-5p, chi-miR-425-3p, chi-miR-425-5p, chi-miR-429, chi-miR-432-3p, chi-miR-432-5p, chi-miR-433, chi-miR-449a-3p, chi-miR-449a-5p, chi-miR-449b-3p, chi-miR-449b-5p, chi-miR-449c, chi-miR-450-3p, chi-miR-450-5p, chi-miR-451-3p, chi-miR-451-5p, chi-miR-454-3p, chi-miR-454-5p, chi-miR-455-3p, chi-miR-455-5p, chi-miR-483, chi-miR-485-3p, chi-miR-485-5p, chi-miR-487a-3p, chi-miR-487a-5p, chi-miR-487b-3p, chi-miR-487b-5p, chi-miR-490, chi-miR-491-3p, chi-miR-491-5p, chi-miR-493-3p, chi-miR-493-5p, chi-miR-494, chi-miR-495-3p, chi-miR-496-3p, chi-miR-496-5p, chi-miR-497-3p, chi-miR-497-5p, chi-miR-499-3p, chi-miR-499-5p, chi-miR-500-3p, chi-miR-500-5p, chi-miR-502a, chi-miR-502b-3p, chi-miR-502b-5p, chi-miR-504, chi-miR-505-3p, chi-miR-532-3p, chi-miR-532-5p, chi-miR-542-3p, chi-miR-542-5p, chi-miR-543-3p, chi-miR-543-5p, chi-miR-544-5p, chi-miR-545-3p, chi-miR-545-5p, chi-miR-582-5p, chi-miR-592, chi-miR-628-5p, chi-miR-655, chi-miR-656, chi-miR-660, chi-miR-665, chi-miR-671-3p, chi-miR-671-5p, chi-miR-7-3p, chi-miR-7-5p, chi-miR-708-3p, chi-miR-708-5p, chi-miR-758, chi-miR-767, chi-miR-873-3p, chi-miR-873-5p, chi-miR-874-3p, chi-miR-874-5p, chi-miR-876-3p, chi-miR-876-5p, chi-miR-877-3p, chi-miR-877-5p, chi-miR-9-3p, chi-miR-9-5p, chi-miR-92a-3p, chi-miR-92a-5p, chi-miR-92b, chi-miR-93-3p, chi-miR-93-5p, chi-miR-96, chi-miR-98-3p, chi-miR-98-5p, chi-miR-99a-3p, chi-miR-99a-5p, chi-miR-99b-3p, chi-miR-99b-5p, cin-let-7a-1-3p, cin-let-7a-2-3p, cin-let-7a-5p, cin-let-7b-3p, cin-let-7b-5p, cin-let-7c, cin-let-7d-3p, cin-let-7d-5p, cin-let-7e, cin-let-7f-3p, cin-let-7f-5p, cin-miR-1-3p, cin-miR-1-5p, cin-miR-101, cin-miR-124-1-5p, cin-miR-124-2-5p, cin-miR-124-3p, cin-miR-125-3p, cin-miR-125-5p, cin-miR-126-3p, cin-miR-126-5p, cin-miR-132-5p, cin-miR-133-3p, cin-miR-133-5p, cin-miR-135-3p, cin-miR-135-5p, cin-miR-141-3p, cin-miR-141-5p, cin-miR-1473-3p, cin-miR-1473-5p, cin-miR-1497-3p, cin-miR-1497-5p, cin-miR-15-3p, cin-miR-15-5p, cin-miR-1502a-3p, cin-miR-1502a-5p, cin-miR-1502b-3p, cin-miR-1502b-5p, cin-miR-1502c-3p, cin-miR-1502c-5p, cin-miR-1502d-3p, cin-miR-1502d-5p, cin-miR-153-3p, cin-miR-153-5p, cin-miR-155, cin-miR-181, cin-miR-182-3p, cin-miR-182-5p, cin-miR-183-3p, cin-miR-183-5p, cin-miR-184, cin-miR-196-3p, cin-miR-196-5p, cin-miR-199-3p, cin-miR-200-3p, cin-miR-200-5p, cin-miR-216, cin-miR-217, cin-miR-219-3p, cin-miR-219-5p, cin-miR-281-3p, cin-miR-281-5p, cin-miR-29-3p, cin-miR-29-5p, cin-miR-31, cin-miR-33, cin-miR-34-3p, cin-miR-34-5p, cin-miR-3575-3p, cin-miR-3575-5p, cin-miR-3598-3p, cin-miR-3598-5p, cin-miR-3599-3p, cin-miR-3599-5p, cin-miR-367-3p, cin-miR-367-5p, cin-miR-375-3p, cin-miR-375-5p, cin-miR-4000a-2-3p, cin-miR-4000a-3p, cin-miR-4000a-5p, cin-miR-4000b-1-3p, cin-miR-4000b-2-3p, cin-miR-4000b-5p, cin-miR-4000c-3p, cin-miR-4000c-5p, cin-miR-4000d-3p, cin-miR-4000d-5p, cin-miR-4000e-3p, cin-miR-4000e-5p, cin-miR-4000f-3p, cin-miR-4000f-5p, cin-miR-4000g-3p, cin-miR-4000g-5p, cin-miR-4000 h-3p, cin-miR-4000 h-5p, cin-miR-4000i-3p, cin-miR-4000i-5p, cin-miR-4001a-1-3p, cin-miR-4001a-2-3p, cin-miR-4001a-5p, cin-miR-4001b-1-3p, cin-miR-4001b-2-3p, cin-miR-4001b-5p, cin-miR-4001c-3p, cin-miR-4001c-5p, cin-miR-4001d-3p, cin-miR-4001d-5p, cin-miR-4001e-3p, cin-miR-4001e-5p, cin-miR-4001f-3p, cin-miR-4001f-5p, cin-miR-4001g-3p, cin-miR-4001 h-3p, cin-miR-4001 h-5p, cin-miR-4001i-3p, cin-miR-4001i-5p, cin-miR-4002-3p, cin-miR-4002-5p, cin-miR-4003a-3p, cin-miR-4003a-5p, cin-miR-4003b-3p, cin-miR-4003b-5p, cin-miR-4003c-3p, cin-miR-4003c-5p, cin-miR-4003d-3p, cin-miR-4004-5p, cin-miR-4005a-3p, cin-miR-4005a-5p, cin-miR-4005b-3p, cin-miR-4005b-5p, cin-miR-4005c-5p, cin-miR-4006a-1-3p, cin-miR-4006a-2-3p, cin-miR-4006a-3-3p, cin-miR-4006a-5p, cin-miR-4006b-3p, cin-miR-4006b-5p, cin-miR-4006c-3p, cin-miR-4006c-5p, cin-miR-4006d-3p, cin-miR-4006d-5p, cin-miR-4006e-3p, cin-miR-4006e-5p, cin-miR-4006f-3p, cin-miR-4006f-5p, cin-miR-4006g-3p, cin-miR-4006g-5p, cin-miR-4007-5p, cin-miR-4008a-3p, cin-miR-4008a-5p, cin-miR-4008b-3p, cin-miR-4008b-5p, cin-miR-4008c-3p, cin-miR-4008c-5p, cin-miR-4009a-3p, cin-miR-4009a-5p, cin-miR-4009b-3p, cin-miR-4009b-5p, cin-miR-4009c-3p, cin-miR-4010-3p, cin-miR-4010-5p, cin-miR-4011a-3p, cin-miR-4011a-5p, cin-miR-4011b-3p, cin-miR-4011b-5p, cin-miR-4012-3p, cin-miR-4012-5p, cin-miR-4013a-3p, cin-miR-4013a-5p, cin-miR-4013b-3p, cin-miR-4013b-5p, cin-miR-4014-1-5p, cin-miR-4014-2-5p, cin-miR-4014-3p, cin-miR-4015-3p, cin-miR-4015-5p, cin-miR-4016, cin-miR-4017-3p, cin-miR-4017-5p, cin-miR-4018a-3p, cin-miR-4018a-5p, cin-miR-4018b-3p, cin-miR-4018b-5p, cin-miR-4019-3p, cin-miR-4019-5p, cin-miR-4020a-5p, cin-miR-4020b-3p, cin-miR-4020b-5p, cin-miR-4021-3p, cin-miR-4021-5p, cin-miR-4022-3p, cin-miR-4023-3p, cin-miR-4024-3p, cin-miR-4024-5p, cin-miR-4025-5p, cin-miR-4026-3p, cin-miR-4026-5p, cin-miR-4027-3p, cin-miR-4027-5p, cin-miR-4028-3p, cin-miR-4029-3p, cin-miR-4029-5p, cin-miR-4030-3p, cin-miR-4030-5p, cin-miR-4031-3p, cin-miR-4031-5p, cin-miR-4032-3p, cin-miR-4032-5p, cin-miR-4033-3p, cin-miR-4033-5p, cin-miR-4034-3p, cin-miR-4034-5p, cin-miR-4035-3p, cin-miR-4035-5p, cin-miR-4036-3p, cin-miR-4036-5p, cin-miR-4037-3p, cin-miR-4037-5p, cin-miR-4038-3p, cin-miR-4038-5p, cin-miR-4039-3p, cin-miR-4039-5p, cin-miR-4040-3p, cin-miR-4040-5p, cin-miR-4041-3p, cin-miR-4041-5p, cin-miR-4042-3p, cin-miR-4042-5p, cin-miR-4043-3p, cin-miR-4043-5p, cin-miR-4044-3p, cin-miR-4044-5p, cin-miR-4045-3p, cin-miR-4045-5p, cin-miR-4046-3p, cin-miR-4046-5p, cin-miR-4047-3p, cin-miR-4047-5p, cin-miR-4048-3p, cin-miR-4048-5p, cin-miR-4049-3p, cin-miR-4049-5p, cin-miR-4050-3p, cin-miR-4050-5p, cin-miR-4051-3p, cin-miR-4051-5p, cin-miR-4052-3p, cin-miR-4052-5p, cin-miR-4053-3p, cin-miR-4053-5p, cin-miR-4054-3p, cin-miR-4054-5p, cin-miR-4055-3p, cin-miR-4055-5p, cin-miR-4056-3p, cin-miR-4056-5p, cin-miR-4057-3p, cin-miR-4057-5p, cin-miR-4058-5p, cin-miR-4059-3p, cin-miR-4059-5p, cin-miR-4060-3p, cin-miR-4060-5p, cin-miR-4061-3p, cin-miR-4061-5p, cin-miR-4062-3p, cin-miR-4062-5p, cin-miR-4063-3p, cin-miR-4063-5p, cin-miR-4064-3p, cin-miR-4064-5p, cin-miR-4065-3p, cin-miR-4065-5p, cin-miR-4066-3p, cin-miR-4066-5p, cin-miR-4067-3p, cin-miR-4067-5p, cin-miR-4068-3p, cin-miR-4068-5p, cin-miR-4069-3p, cin-miR-4069-5p, cin-miR-4070-3p, cin-miR-4070-5p, cin-miR-4071-3p, cin-miR-4071-5p, cin-miR-4072-3p, cin-miR-4072-5p, cin-miR-4073-3p, cin-miR-4073-5p, cin-miR-4074-3p, cin-miR-4074-5p, cin-miR-4075-3p, cin-miR-4075-5p, cin-miR-4076-3p, cin-miR-4076-5p, cin-miR-4077a-5p, cin-miR-4077b-5p, cin-miR-4077c-5p, cin-miR-4077d-5p, cin-miR-4078-3p, cin-miR-4078-5p, cin-miR-4079-3p, cin-miR-4079-5p, cin-miR-4080-3p, cin-miR-4080-5p, cin-miR-4081-3p, cin-miR-4081-5p, cin-miR-4082-3p, cin-miR-4082-5p, cin-miR-4083-3p, cin-miR-4083-5p, cin-miR-4084-3p, cin-miR-4084-5p, cin-miR-4085-3p, cin-miR-4085-5p, cin-miR-4086-3p, cin-miR-4087-3p, cin-miR-4087-5p, cin-miR-4088-3p, cin-miR-4088-5p, cin-miR-4089-5p, cin-miR-4090-3p, cin-miR-4091-3p, cin-miR-4091-5p, cin-miR-4092-3p, cin-miR-4092-5p, cin-miR-4093-3p, cin-miR-4093-5p, cin-miR-4094-3p, cin-miR-4094-5p, cin-miR-4095-5p, cin-miR-4096-3p, cin-miR-4097-3p, cin-miR-4097-5p, cin-miR-4098-3p, cin-miR-4098-5p, cin-miR-4099-3p, cin-miR-4099-5p, cin-miR-4100-3p, cin-miR-4100-5p, cin-miR-4101-3p, cin-miR-4102-3p, cin-miR-4102-5p, cin-miR-4103-3p, cin-miR-4104-5p, cin-miR-4105-5p, cin-miR-4106-3p, cin-miR-4107-5p, cin-miR-4108-5p, cin-miR-4109-3p, cin-miR-4109-5p, cin-miR-4110-3p, cin-miR-4110-5p, cin-miR-4111-3p, cin-miR-4112-3p, cin-miR-4112-5p, cin-miR-4113-5p, cin-miR-4114-5p, cin-miR-4115-3p, cin-miR-4115-5p, cin-miR-4116-5p, cin-miR-4117-5p, cin-miR-4118-3p, cin-miR-4119-3p, cin-miR-4119-5p, cin-miR-4120-5p, cin-miR-4121-3p, cin-miR-4122-3p, cin-miR-4123-3p, cin-miR-4123-5p, cin-miR-4124-5p, cin-miR-4125-5p, cin-miR-4126-5p, cin-miR-4127-3p, cin-miR-4127-5p, cin-miR-4128-3p, cin-miR-4129-3p, cin-miR-4130-3p, cin-miR-4131-3p, cin-miR-4131-5p, cin-miR-4132-3p, cin-miR-4132-5p, cin-miR-4133-3p, cin-miR-4134-5p, cin-miR-4135-3p, cin-miR-4136-3p, cin-miR-4137-3p, cin-miR-4138-5p, cin-miR-4139-3p, cin-miR-4139-5p, cin-miR-4140-3p, cin-miR-4140-5p, cin-miR-4141-3p, cin-miR-4141-5p, cin-miR-4142-3p, cin-miR-4142-5p, cin-miR-4143-3p, cin-miR-4143-5p, cin-miR-4144-3p, cin-miR-4144-5p, cin-miR-4145-3p, cin-miR-4146-3p, cin-miR-4147-3p, cin-miR-4148-5p, cin-miR-4149-5p, cin-miR-4150-5p, cin-miR-4151-3p, cin-miR-4151-5p, cin-miR-4152-5p, cin-miR-4153-3p, cin-miR-4154-3p, cin-miR-4155-3p, cin-miR-4155-5p, cin-miR-4156-3p, cin-miR-4156-5p, cin-miR-4157-5p, cin-miR-4158-5p, cin-miR-4159-3p, cin-miR-4160-3p, cin-miR-4162-3p, cin-miR-4162-5p, cin-miR-4163-3p, cin-miR-4163-5p, cin-miR-4164-3p, cin-miR-4165-3p, cin-miR-4166-5p, cin-miR-4167-5p, cin-miR-4168-3p, cin-miR-4169-3p, cin-miR-4170-5p, cin-miR-4171-5p, cin-miR-4172-3p, cin-miR-4173-3p, cin-miR-4173-5p, cin-miR-4174-3p, cin-miR-4175-3p, cin-miR-4176-5p, cin-miR-4177-3p, cin-miR-4177-5p, cin-miR-4178a-5p, cin-miR-4178b-5p, cin-miR-4179a-3p, cin-miR-4179a-5p, cin-miR-4179b-3p, cin-miR-4179b-5p, cin-miR-4180-3p, cin-miR-4180-5p, cin-miR-4181-3p, cin-miR-4181-5p, cin-miR-4182-3p, cin-miR-4182-5p, cin-miR-4183-3p, cin-miR-4183-5p, cin-miR-4184-3p, cin-miR-4185-3p, cin-miR-4185-5p, cin-miR-4186-5p, cin-miR-4187-5p, cin-miR-4188-5p, cin-miR-4189-5p, cin-miR-4190-5p, cin-miR-4191-5p, cin-miR-4192-3p, cin-miR-4193-5p, cin-miR-4194-3p, cin-miR-4194-5p, cin-miR-4195-3p, cin-miR-4196-5p, cin-miR-4197-5p, cin-miR-4198-5p, cin-miR-4199-3p, cin-miR-4200-3p, cin-miR-4200-5p, cin-miR-4201-5p, cin-miR-4202-5p, cin-miR-4203-3p, cin-miR-4204-3p, cin-miR-4204-5p, cin-miR-4205-5p, cin-miR-4206-3p, cin-miR-4207-3p, cin-miR-4208-3p, cin-miR-4209-5p, cin-miR-4210-5p, cin-miR-4211-5p, cin-miR-4212-3p, cin-miR-4213-5p, cin-miR-4214-5p, cin-miR-4215-3p, cin-miR-4216-3p, cin-miR-4216-5p, cin-miR-4217-3p, cin-miR-4218-5p, cin-miR-4219-5p, cin-miR-4220-3p, cin-miR-4220-5p, cin-miR-5596a-3p, cin-miR-5596a-5p, cin-miR-5596b-3p, cin-miR-5596b-5p, cin-miR-5597-3p, cin-miR-5597-5p, cin-miR-5598-3p, cin-miR-5598-5p, cin-miR-5599-3p, cin-miR-5599-5p, cin-miR-5600-3p, cin-miR-5600-5p, cin-miR-5601-3p, cin-miR-5601-5p, cin-miR-5602-3p, cin-miR-5602-5p, cin-miR-5603-3p, cin-miR-5603-5p, cin-miR-5604-3p, cin-miR-5604-5p, cin-miR-5605-3p, cin-miR-5605-5p, cin-miR-5606-3p, cin-miR-5606-5p, cin-miR-5607-3p, cin-miR-5607-5p, cin-miR-5608-3p, cin-miR-5608-5p, cin-miR-5609-3p, cin-miR-5609-5p, cin-miR-5610-3p, cin-miR-5610-5p, cin-miR-5611-3p, cin-miR-5611-5p, cin-miR-5612-3p, cin-miR-5612-5p, cin-miR-672, cin-miR-7-3p, cin-miR-7-5p, cin-miR-78, cin-miR-9-5p, cin-miR-92a-3p, cin-miR-92a-5p, cin-miR-92b-3p, cin-miR-92b-5p, cin-miR-92c-3p, cin-miR-92c-5p, cin-miR-92d-3p, cin-miR-92d-5p, cin-miR-92e-3p, cin-miR-92e-5p, cin-miR-96-3p, cin-miR-96-5p, cja-let-7a, cja-let-7b, cja-let-7c, cja-let-7d, cja-let-7e, cja-let-7f, cja-let-7g, cja-let-7i, cja-miR-100, cja-miR-101, cja-miR-101-2, cja-miR-103a, cja-miR-103b, cja-miR-105, cja-miR-106b, cja-miR-10a, cja-miR-10b, cja-miR-1185, cja-miR-1193, cja-miR-122, cja-miR-124, cja-miR-1240, cja-miR-1247, cja-miR-1249, cja-miR-125, cja-miR-1251, cja-miR-1253, cja-miR-1258a, cja-miR-1258b, cja-miR-125a, cja-miR-125b, cja-miR-126, cja-miR-1264, cja-miR-127, cja-miR-128, cja-miR-1282, cja-miR-129, cja-miR-1294, cja-miR-1296, cja-miR-1298, cja-miR-1301, cja-miR-1302, cja-miR-1305, cja-miR-1306, cja-miR-1307, cja-miR-130a, cja-miR-130b, cja-miR-132, cja-miR-1323, cja-miR-1327, cja-miR-133a, cja-miR-133b, cja-miR-134, cja-miR-135, cja-miR-137, cja-miR-138, cja-miR-139, cja-miR-140, cja-miR-141, cja-miR-142, cja-miR-143, cja-miR-144, cja-miR-145, cja-miR-1468, cja-miR-146a, cja-miR-146b, cja-miR-148a, cja-miR-148b, cja-miR-150, cja-miR-151, cja-miR-152, cja-miR-153, cja-miR-154, cja-miR-155, cja-miR-15b, cja-miR-16, cja-miR-17, cja-miR-181a, cja-miR-181b, cja-miR-181c, cja-miR-181d, cja-miR-182, cja-miR-183, cja-miR-184, cja-miR-185, cja-miR-186, cja-miR-187, cja-miR-1895, cja-miR-1898, cja-miR-190a, cja-miR-190b, cja-miR-191, cja-miR-1911, cja-miR-193, cja-miR-194, cja-miR-196a, cja-miR-196b, cja-miR-197, cja-miR-199, cja-miR-19a, cja-miR-19b, cja-miR-1a, cja-miR-1b-3p, cja-miR-1b-5p, cja-miR-200c, cja-miR-202, cja-miR-203, cja-miR-204, cja-miR-205, cja-miR-2053, cja-miR-206, cja-miR-208a, cja-miR-208b, cja-miR-20a, cja-miR-20b, cja-miR-21, cja-miR-211, cja-miR-2114, cja-miR-214, cja-miR-215, cja-miR-216a, cja-miR-216b, cja-miR-217, cja-miR-218, cja-miR-219, cja-miR-22, cja-miR-222, cja-miR-223, cja-miR-224, cja-miR-2338, cja-miR-2355, cja-miR-23a, cja-miR-23b, cja-miR-24, cja-miR-25, cja-miR-26a, cja-miR-26b, cja-miR-27a, cja-miR-27b, cja-miR-28, cja-miR-296, cja-miR-2968, cja-miR-299, cja-miR-29a, cja-miR-29b, cja-miR-29c, cja-miR-301a, cja-miR-302a-3p, cja-miR-302a-5p, cja-miR-302b, cja-miR-302c, cja-miR-302d-3p, cja-miR-302d-5p, cja-miR-3065, cja-miR-3074, cja-miR-30a, cja-miR-30b, cja-miR-30c, cja-miR-30d, cja-miR-30e, cja-miR-31, cja-miR-3118, cja-miR-3120, cja-miR-3135, cja-miR-3152, cja-miR-32, cja-miR-320, cja-miR-323, cja-miR-324, cja-miR-328, cja-miR-329, cja-miR-33, cja-miR-330, cja-miR-331, cja-miR-335-3p, cja-miR-335-5p, cja-miR-337, cja-miR-338, cja-miR-339, cja-miR-340, cja-miR-342, cja-miR-345, cja-miR-34a, cja-miR-34b, cja-miR-34c, cja-miR-3544, cja-miR-3546, cja-miR-3576, cja-miR-361, cja-miR-363, cja-miR-365, cja-miR-367, cja-miR-3688, cja-miR-369-3p, cja-miR-369-5p, cja-miR-371a, cja-miR-371b-3p, cja-miR-371b-5p, cja-miR-372, cja-miR-373, cja-miR-374a, cja-miR-374b, cja-miR-376a-3p, cja-miR-376a-5p, cja-miR-376b, cja-miR-377, cja-miR-378, cja-miR-379, cja-miR-381, cja-miR-382, cja-miR-383a, cja-miR-383b, cja-miR-3940, cja-miR-409, cja-miR-410, cja-miR-411, cja-miR-412, cja-miR-421, cja-miR-423-3p, cja-miR-423-5p, cja-miR-424, cja-miR-429, cja-miR-432, cja-miR-4460, cja-miR-448, cja-miR-449a, cja-miR-449c, cja-miR-4503, cja-miR-450a, cja-miR-450b, cja-miR-450c, cja-miR-451, cja-miR-4512, cja-miR-452, cja-miR-455, cja-miR-466a, cja-miR-466b, cja-miR-4679, cja-miR-4683, cja-miR-4745, cja-miR-4762, cja-miR-4772, cja-miR-4780, cja-miR-4790, cja-miR-483, cja-miR-484, cja-miR-485, cja-miR-487a, cja-miR-487b, cja-miR-488, cja-miR-489, cja-miR-491, cja-miR-493, cja-miR-494, cja-miR-495, cja-miR-497, cja-miR-498, cja-miR-499a, cja-miR-499b, cja-miR-502b, cja-miR-503, cja-miR-504, cja-miR-505, cja-miR-506, cja-miR-507, cja-miR-508, cja-miR-509, cja-miR-510a, cja-miR-510b, cja-miR-513a-3p, cja-miR-513a-5p, cja-miR-513b-5p, cja-miR-513c-5p, cja-miR-513d-5p, cja-miR-513e-3p, cja-miR-513f-3p, cja-miR-513f-5p, cja-miR-513g-3p, cja-miR-513 h-3p, cja-miR-514a, cja-miR-514b, cja-miR-518a, cja-miR-518b, cja-miR-5191, cja-miR-519a, cja-miR-519b, cja-miR-519c, cja-miR-521, cja-miR-523, cja-miR-532, cja-miR-539, cja-miR-541, cja-miR-542-3p, cja-miR-542-5p, cja-miR-543, cja-miR-544, cja-miR-545, cja-miR-548a, cja-miR-548b, cja-miR-548c, cja-miR-548d, cja-miR-548e, cja-miR-548f, cja-miR-548g, cja-miR-548h, cja-miR-548i, cja-miR-548j, cja-miR-548k, cja-miR-5481, cja-miR-548m, cja-miR-548n, cja-miR-5480, cja-miR-548p, cja-miR-548q, cja-miR-548r, cja-miR-548s, cja-miR-548t, cja-miR-548u, cja-miR-548v, cja-miR-548w, cja-miR-548x, cja-miR-548y, cja-miR-551, cja-miR-556, cja-miR-557, cja-miR-559, cja-miR-561, cja-miR-569, cja-miR-577, cja-miR-580, cja-miR-581, cja-miR-582, cja-miR-584, cja-miR-586, cja-miR-589, cja-miR-598, cja-miR-612, cja-miR-615, cja-miR-616, cja-miR-617, cja-miR-622, cja-miR-628, cja-miR-631, cja-miR-638, cja-miR-651, cja-miR-6529, cja-miR-652a, cja-miR-652b, cja-miR-653, cja-miR-654, cja-miR-655, cja-miR-656, cja-miR-660, cja-miR-665, cja-miR-670, cja-miR-671, cja-miR-675, cja-miR-676, cja-miR-7, cja-miR-708, cja-miR-759, cja-miR-761, cja-miR-763, cja-miR-767, cja-miR-769, cja-miR-770, cja-miR-873, cja-miR-875-3p, cja-miR-875-5p, cja-miR-876, cja-miR-885, cja-miR-887-3p, cja-miR-887-5p, cja-miR-889, cja-miR-891b, cja-miR-891c, cja-miR-891d, cja-miR-9, cja-miR-92a, cja-miR-92b, cja-miR-93, cja-miR-934, cja-miR-942, cja-miR-95, cja-miR-96, cja-miR-98, cja-miR-99, cja-miR-9904, cja-miR-9905, cja-miR-9906, cja-miR-9907, cja-miR-9908, cja-miR-9909, cja-miR-9910, cja-miR-9911, cja-miR-9912, cja-miR-9913, cja-miR-9914, cja-miR-9915, cja-miR-9916, cja-miR-9917, cja-miR-9918, cja-miR-9919, cja-miR-9921, cja-miR-9922, cja-miR-9923, cja-miR-9924, cja-miR-9925, cja-miR-9926, cja-miR-9927, cja-miR-9928, cja-miR-9929, cja-miR-9930, cja-miR-9931, cja-miR-9932, cja-miR-9933, cja-miR-9934, cja-miR-9935, cja-miR-9936, cja-miR-9937, cja-miR-9938, cja-miR-9939, cja-miR-9940, cja-miR-9941, cja-miR-9942, cja-miR-9943, cja-miR-9944, cja-miR-9945, cja-miR-9946, cja-miR-9947, cja-miR-9948a, cja-miR-9948b, cja-miR-9949, cja-miR-9950, cja-miR-9951, cja-miR-9952a, cja-miR-9952b, cja-miR-9952c, cja-miR-9953, cja-miR-9954, cja-miR-9955, cja-miR-9956, cja-miR-9957, cja-miR-9958, cja-miR-9959, cja-miR-9960, cja-miR-9961, cja-miR-9962, cja-miR-9963, cja-miR-9964, cja-miR-9965, cja-miR-9966, cja-miR-9967, cja-miR-9968, cja-miR-9969, cja-miR-9970, cja-miR-9971, cja-miR-9972, cja-miR-9973, cja-miR-9974, cja-miR-9975, cja-miR-9976, cja-miR-9977, cja-miR-9978, cja-miR-9979, cja-miR-9980, cja-miR-9981, cja-miR-9982, cja-miR-9983, cja-miR-9984, cla-miR-1992, cla-miR-1994, cli-let-7a-2-3p, cli-let-7a-3p, cli-let-7a-4-3p, cli-let-7a-5p, cli-let-7b-3p, cli-let-7c-3p, cli-let-7c-5p, cli-let-7d-3p, cli-let-7d-5p, cli-let-7e-3p, cli-let-7e-5p, cli-let-7f-3p, cli-let-7f-5p, cli-let-7g-3p, cli-let-7g-5p, cli-let-7i-3p, cli-let-7i-5p, cli-miR-100-3p, cli-miR-100-5p, cli-miR-101-2-5p, cli-miR-101-3p, cli-miR-101-5p, cli-miR-103-3p, cli-miR-103-5p, cli-miR-106-3p, cli-miR-106-5p, cli-miR-107-3p, cli-miR-107-5p, cli-miR-10a-3p, cli-miR-10a-5p, cli-miR-10b-3p, cli-miR-10b-5p, cli-miR-10c-3p, cli-miR-10c-5p, cli-miR-122-3p, cli-miR-122-5p, cli-miR-124-2-5p, cli-miR-124-3p, cli-miR-124-5p, cli-miR-125-2-3p, cli-miR-125-3p, cli-miR-125-5p, cli-miR-126-3p, cli-miR-126-5p, cli-miR-128-3p, cli-miR-128-5p, cli-miR-129-3p, cli-miR-129-5p, cli-miR-1306-3p, cli-miR-1306-5p, cli-miR-130a-3p, cli-miR-130a-5p, cli-miR-130c-3p, cli-miR-130c-5p, cli-miR-1329-3p, cli-miR-1329-5p, cli-miR-133a-3p, cli-miR-133a-5p, cli-miR-133b-3p, cli-miR-133b-5p, cli-miR-133c-3p, cli-miR-135-2-3p, cli-miR-135-3-3p, cli-miR-135-3p, cli-miR-135-5p, cli-miR-137a-3p, cli-miR-137a-5p, cli-miR-137b-3p, cli-miR-137b-5p, cli-miR-138-2-3p, cli-miR-138-3p, cli-miR-138-5p, cli-miR-1388-3p, cli-miR-1388-5p, cli-miR-139-3p, cli-miR-139-5p, cli-miR-140-3p, cli-miR-140-5p, cli-miR-1416-3p, cli-miR-1416-5p, cli-miR-142-3p, cli-miR-142-5p, cli-miR-143-3p, cli-miR-143-5p, cli-miR-145-3p, cli-miR-145-5p, cli-miR-1451-3p, cli-miR-1451-5p, cli-miR-1467-3p, cli-miR-1467-5p, cli-miR-146a-3p, cli-miR-146a-5p, cli-miR-146b-3p, cli-miR-146b-5p, cli-miR-146c-3p, cli-miR-146c-5p, cli-miR-147-3p, cli-miR-148a-3p, cli-miR-148a-5p, cli-miR-148b-3p, cli-miR-153a-3p, cli-miR-153a-5p, cli-miR-153b-3p, cli-miR-153b-5p, cli-miR-155-5p, cli-miR-1550-5p, cli-miR-1552-3p, cli-miR-1552-5p, cli-miR-1559-3p, cli-miR-1559-5p, cli-miR-15a-3p, cli-miR-15a-5p, cli-miR-15b-3p, cli-miR-15b-5p, cli-miR-15c-3p, cli-miR-15c-5p, cli-miR-1641-5p, cli-miR-1655-3p, cli-miR-1662-3p, cli-miR-1662-5p, cli-miR-1677-3p, cli-miR-1677-5p, cli-miR-16a-2-3p, cli-miR-16a-3p, cli-miR-16a-5p, cli-miR-16b-3p, cli-miR-16b-5p, cli-miR-17-3p, cli-miR-17-5p, cli-miR-1720-3p, cli-miR-1720-5p, cli-miR-1729-3p, cli-miR-1729-5p, cli-miR-1756a-3p, cli-miR-1756b-3p, cli-miR-1756b-5p, cli-miR-1781-3p, cli-miR-1782-3p, cli-miR-1784-5p, cli-miR-1788-3p, cli-miR-1788-5p, cli-miR-1791-3p, cli-miR-1803-3p, cli-miR-1803-5p, cli-miR-1805-3p, cli-miR-1805-5p, cli-miR-181a-2-3p, cli-miR-181a-3p, cli-miR-181a-5p, cli-miR-181b-2-3p, cli-miR-181b-3p, cli-miR-181b-5p, cli-miR-182-3p, cli-miR-182-5p, cli-miR-183-3p, cli-miR-183-5p, cli-miR-184-3p, cli-miR-184-5p, cli-miR-187-3p, cli-miR-187-5p, cli-miR-18a-3p, cli-miR-18a-5p, cli-miR-18b-3p, cli-miR-18b-5p, cli-miR-190-3p, cli-miR-190-5p, cli-miR-193-3p, cli-miR-193-5p, cli-miR-194-3p, cli-miR-194-5p, cli-miR-196-3-3p, cli-miR-196-3p, cli-miR-196-5p, cli-miR-199-3p, cli-miR-199-5p, cli-miR-19a-3p, cli-miR-19a-5p, cli-miR-19b-2-5p, cli-miR-19b-3p, cli-miR-19b-5p, cli-miR-1a-2-5p, cli-miR-1a-3p, cli-miR-1a-5p, cli-miR-1b-3p, cli-miR-1b-5p, cli-miR-200a-3p, cli-miR-200a-5p, cli-miR-200b-3p, cli-miR-200b-5p, cli-miR-202-5p, cli-miR-204-2-3p, cli-miR-204-3-3p, cli-miR-204-3p, cli-miR-204-5p, cli-miR-205a-3p, cli-miR-205a-5p, cli-miR-205b-3p, cli-miR-205b-5p, cli-miR-206-3p, cli-miR-206-5p, cli-miR-20a-3p, cli-miR-20a-5p, cli-miR-20b-3p, cli-miR-20b-5p, cli-miR-21-3p, cli-miR-21-5p, cli-miR-210-3p, cli-miR-210-5p, cli-miR-2131-3p, cli-miR-2131-5p, cli-miR-214-3p, cli-miR-214-5p, cli-miR-215-3p, cli-miR-215-5p, cli-miR-216a-3p, cli-miR-216a-5p, cli-miR-216b-3p, cli-miR-216b-5p, cli-miR-217-3p, cli-miR-217-5p, cli-miR-218-3p, cli-miR-218-5p, cli-miR-2188-3p, cli-miR-2188-5p, cli-miR-22-3p, cli-miR-22-5p, cli-miR-221-3p, cli-miR-221-5p, cli-miR-222a-3p, cli-miR-222a-5p, cli-miR-222b-3p, cli-miR-223-3p, cli-miR-223-5p, cli-miR-23b-3p, cli-miR-23b-5p, cli-miR-26-2-3p, cli-miR-26-3p, cli-miR-26-5p, cli-miR-27b-3p, cli-miR-27b-5p, cli-miR-2954-3p, cli-miR-2954-5p, cli-miR-2970-3p, cli-miR-2970-5p, cli-miR-2984-5p, cli-miR-29a-3p, cli-miR-29a-5p, cli-miR-29b-3p, cli-miR-29b-5p, cli-miR-301a-3p, cli-miR-301a-5p, cli-miR-301b-3p, cli-miR-301b-5p, cli-miR-302a-3p, cli-miR-302b-3p, cli-miR-302c-3p, cli-miR-3064-3p, cli-miR-30a-3p, cli-miR-30a-5p, cli-miR-30c-2-3p, cli-miR-30c-3p, cli-miR-30c-5p, cli-miR-30d-3p, cli-miR-30d-5p, cli-miR-30e-3p, cli-miR-30e-5p, cli-miR-31-3p, cli-miR-31-5p, cli-miR-32-3p, cli-miR-32-5p, cli-miR-33-2-3p, cli-miR-33-3p, cli-miR-33-5p, cli-miR-338a-3p, cli-miR-338a-5p, cli-miR-338b-3p, cli-miR-338b-5p, cli-miR-34a-3p, cli-miR-34a-5p, cli-miR-34b-3p, cli-miR-34b-5p, cli-miR-34c-3p, cli-miR-34c-5p, cli-miR-3618-3p, cli-miR-3618-5p, cli-miR-363-3p, cli-miR-363-5p, cli-miR-365-2-5p, cli-miR-365-3p, cli-miR-365-5p, cli-miR-367-3p, cli-miR-375-3p, cli-miR-375-5p, cli-miR-383-3p, cli-miR-383-5p, cli-miR-425-3p, cli-miR-425-5p, cli-miR-429-3p, cli-miR-429-5p, cli-miR-449a-5p, cli-miR-449b-3p, cli-miR-449b-5p, cli-miR-449c-3p, cli-miR-449c-5p, cli-miR-449d-3p, cli-miR-449d-5p, cli-miR-451-5p, cli-miR-454-3p, cli-miR-454-5p, cli-miR-455-3p, cli-miR-455-5p, cli-miR-456-3p, cli-miR-458-3p, cli-miR-458-5p, cli-miR-459-5p, cli-miR-460a-3p, cli-miR-460a-5p, cli-miR-460b-3p, cli-miR-460b-5p, cli-miR-489-3p, cli-miR-489-5p, cli-miR-490-3p, cli-miR-490-5p, cli-miR-499-3p, cli-miR-499-5p, cli-miR-551a-3p, cli-miR-551a-5p, cli-miR-551b-3p, cli-miR-551b-5p, cli-miR-599-3p, cli-miR-737-5p, cli-miR-7a-2-3p, cli-miR-7a-3-3p, cli-miR-7a-3p, cli-miR-7a-5p, cli-miR-7b-5p, cli-miR-875-3p, cli-miR-875-5p, cli-miR-8993-3p, cli-miR-8993-5p, cli-miR-9-3-3p, cli-miR-9-3p, cli-miR-9-5p, cli-miR-92-2-5p, cli-miR-92-3p, cli-miR-92-5p, cli-miR-96-3p, cli-miR-96-5p, cli-miR-9615-3p, cli-miR-9615-5p, cli-miR-9616-3p, cli-miR-9616-5p, cli-miR-9617-3p, cli-miR-9617-5p, cli-miR-9618-3p, cli-miR-9618-5p, cli-miR-9619-3p, cli-miR-9619-5p, cli-miR-9620-3p, cli-miR-9620-5p, cli-miR-9621-3p, cli-miR-9621-5p, cli-miR-9622-3p, cli-miR-9622-5p, cli-miR-9623-3p, cli-miR-9623-5p, cli-miR-9624-3p, cli-miR-9624-5p, cli-miR-9625-3p, cli-miR-9625-5p, cli-miR-9626-3p, cli-miR-9626-5p, cli-miR-9627-3p, cli-miR-9627-5p, cli-miR-9628-3p, cli-miR-9628-5p, cli-miR-9629-3p, cli-miR-9629-5p, cli-miR-9630-3p, cli-miR-9630-5p, cli-miR-9631-3p, cli-miR-9631-5p, cli-miR-9632-3p, cli-miR-9632-5p, cli-miR-9633-3p, cli-miR-9633-5p, cli-miR-9634-3p, cli-miR-9634-5p, cli-miR-9635-3p, cli-miR-9635-5p, cli-miR-9636-3p, cli-miR-9636-5p, cli-miR-9637-3p, cli-miR-9637-5p, cli-miR-9638-3p, cli-miR-9638-5p, cli-miR-9639-3p, cli-miR-9639-5p, cli-miR-9640-3p, cli-miR-9640-5p, cli-miR-9641-3p, cli-miR-9641-5p, cli-miR-9642-3p, cli-miR-9642-5p, cli-miR-9643-3p, cli-miR-9643-5p, cli-miR-9644-3p, cli-miR-9644-5p, cli-miR-9645-3p, cli-miR-9645-5p, cli-miR-9646-3p, cli-miR-9646-5p, cli-miR-9647-3p, cli-miR-9647-5p, cli-miR-9648-3p, cli-miR-9648-5p, cli-miR-9649-3p, cli-miR-9649-5p, cli-miR-99-3p, cli-miR-99-5p, cln-miR162, cln-miR164, cln-miR166, cln-miR6725, cme-miR156a, cme-miR156b, cme-miR156c, cme-miR156d, cme-miR156e, cme-miR156f, cme-miR156g, cme-miR156h, cme-miR156i, cme-miR156j, cme-miR159a, cme-miR159b, cme-miR160a, cme-miR160b, cme-miR160c, cme-miR160d, cme-miR162, cme-miR164a, cme-miR164b, cme-miR164c, cme-miR164d, cme-miR166a, cme-miR166b, cme-miR166c, cme-miR166d, cme-miR166e, cme-miR166f, cme-miR 166g, cme-miR166h, cme-miR 166i, cme-miR167a, cme-miR167b, cme-miR167c, cme-miR167d, cme-miR167e, cme-miR167f, cme-miR168, cme-miR169a, cme-miR169b, cme-miR169c, cme-miR169d, cme-miR169e, cme-miR169f, cme-miR169g, cme-miR169h, cme-miR169i, cme-miR169j, cme-miR169k, cme-miR1691, cme-miR169m, cme-miR169n, cme-miR1690, cme-miR169p, cme-miR169q, cme-miR169r, cme-miR169s, cme-miR169t, cme-miR171a, cme-miR171b, cme-miR171c, cme-miR171d, cme-miR171e, cme-miR171f, cme-miR171g, cme-miR171h, cme-miR171i, cme-miR172a, cme-miR172b, cme-miR172c, cme-miR172d, cme-miR172e, cme-miR172f, cme-miR1863, cme-miR2111a, cme-miR2111b, cme-miR319a, cme-miR319b, cme-miR319c, cme-miR319d, cme-miR390a, cme-miR390b, cme-miR390c, cme-miR390d, cme-miR393a, cme-miR393b, cme-miR393c, cme-miR394a, cme-miR394b, cme-miR395a, cme-miR395b, cme-miR395c, cme-miR395d, cme-miR395e, cme-miR395f, cme-miR396a, cme-miR396b, cme-miR396c, cme-miR396d, cme-miR396e, cme-miR397, cme-miR398a, cme-miR398b, cme-miR399a, cme-miR399b, cme-miR399c, cme-miR399d, cme-miR399e, cme-miR399f, cme-miR399g, cme-miR408, cme-miR477a, cme-miR477b, cme-miR530a, cme-miR530b, cme-miR7129, cme-miR7130, cme-miR828, cme-miR845, cme-miR854, cme-miR858, cpa-miR156a, cpa-miR156b, cpa-miR156c, cpa-miR156d, cpa-miR156e, cpa-miR156f, cpa-miR159a, cpa-miR159b, cpa-miR160a, cpa-miR160b, cpa-miR160c-3p, cpa-miR160c-5p, cpa-miR160d, cpa-miR160e, cpa-miR160f-3p, cpa-miR160f-5p, cpa-miR162a, cpa-miR164a, cpa-miR164b, cpa-miR164c, cpa-miR164d, cpa-miR164e, cpa-miR166a, cpa-miR166b, cpa-miR166c, cpa-miR166d, cpa-miR166e, cpa-miR167a, cpa-miR167b, cpa-miR167c, cpa-miR167d, cpa-miR169, cpa-miR171a, cpa-miR171b, cpa-miR171c, cpa-miR171d, cpa-miR172a, cpa-miR172b, cpa-miR319, cpa-miR390a, cpa-miR390b, cpa-miR393, cpa-miR394a, cpa-miR394b, cpa-miR395a, cpa-miR395b, cpa-miR395c, cpa-miR395d, cpa-miR395e, cpa-miR396, cpa-miR398, cpa-miR408, cpa-miR477, cpa-miR5211, cpa-miR535, cpa-miR8134, cpa-miR8135, cpa-miR8136, cpa-miR8137, cpa-miR8138, cpa-miR8139a, cpa-miR8139b, cpa-miR8139c, cpa-miR8139d, cpa-miR8139e, cpa-miR8140, cpa-miR8141, cpa-miR8142, cpa-miR8143, cpa-miR8144, cpa-miR8145, cpa-miR8146, cpa-miR8147, cpa-miR8148, cpa-miR8149, cpa-miR8150, cpa-miR8151, cpa-miR8152, cpa-miR8153, cpa-miR8154, cpa-miR8155, cpi-let-7a-2-3p, cpi-let-7a-3p, cpi-let-7a-4-3p, cpi-let-7a-5p, cpi-let-7b-5p, cpi-let-7c-2-3p, cpi-let-7c-3p, cpi-let-7c-5p, cpi-let-7d-3p, cpi-let-7d-5p, cpi-let-7e-3p, cpi-let-7e-5p, cpi-let-7f-3p, cpi-let-7f-5p, cpi-let-7g-3p, cpi-let-7g-5p, cpi-let-7i-3p, cpi-let-7i-5p, cpi-miR-100-3p, cpi-miR-100-5p, cpi-miR-101-2-5p, cpi-miR-101-3p, cpi-miR-101-5p, cpi-miR-103-3p, cpi-miR-103-5p, cpi-miR-106a-5p, cpi-miR-106b-5p, cpi-miR-107-3p, cpi-miR-10a-3p, cpi-miR-10a-5p, cpi-miR-10b-3p, cpi-miR-10b-5p, cpi-miR-10c-3p, cpi-miR-10c-5p, cpi-miR-122-3p, cpi-miR-122-5p, cpi-miR-124-3p, cpi-miR-124-5p, cpi-miR-125a-3p, cpi-miR-125a-5p, cpi-miR-125b-2-3p, cpi-miR-125b-3p, cpi-miR-125b-5p, cpi-miR-126-3p, cpi-miR-126-5p, cpi-miR-128-2-5p, cpi-miR-128-3p, cpi-miR-128-5p, cpi-miR-129-2-3p, cpi-miR-129-3p, cpi-miR-129-5p, cpi-miR-1306-3p, cpi-miR-1306-5p, cpi-miR-130a-3p, cpi-miR-130a-5p, cpi-miR-130b-3p, cpi-miR-130b-5p, cpi-miR-130c-3p, cpi-miR-130c-5p, cpi-miR-130d-3p, cpi-miR-132-3p, cpi-miR-132-5p, cpi-miR-1329-5p, cpi-miR-133a-3p, cpi-miR-133a-5p, cpi-miR-133b-3p, cpi-miR-133c-3p, cpi-miR-135-2-3p, cpi-miR-135-3-3p, cpi-miR-135-3p, cpi-miR-135-5p, cpi-miR-137a-3p, cpi-miR-137b-3p, cpi-miR-138-3p, cpi-miR-138-5p, cpi-miR-1388-3p, cpi-miR-1388-5p, cpi-miR-139-3p, cpi-miR-139-5p, cpi-miR-1397-5p, cpi-miR-140-3p, cpi-miR-140-5p, cpi-miR-1416-5p, cpi-miR-142-3p, cpi-miR-142-5p, cpi-miR-143-3p, cpi-miR-143-5p, cpi-miR-144-3p, cpi-miR-144-5p, cpi-miR-145-3p, cpi-miR-145-5p, cpi-miR-146a-3p, cpi-miR-146a-5p, cpi-miR-146b-3p, cpi-miR-146b-5p, cpi-miR-146c-3p, cpi-miR-146c-5p, cpi-miR-147-3p, cpi-miR-147-5p, cpi-miR-148a-3p, cpi-miR-148a-5p, cpi-miR-148b-3p, cpi-miR-152-3p, cpi-miR-152-5p, cpi-miR-153-3p, cpi-miR-153-5p, cpi-miR-155a-5p, cpi-miR-155b-5p, cpi-miR-15a-5p, cpi-miR-15b-3p, cpi-miR-15b-5p, cpi-miR-15c-3p, cpi-miR-15c-5p, cpi-miR-1641-5p, cpi-miR-1662-3p, cpi-miR-1662-5p, cpi-miR-1677a-3p, cpi-miR-1677a-5p, cpi-miR-1677b-3p, cpi-miR-1677b-5p, cpi-miR-1677c-3p, cpi-miR-1677c-5p, cpi-miR-1677d-3p, cpi-miR-1677d-5p, cpi-miR-1677e-3p, cpi-miR-1677e-5p, cpi-miR-1677f-3p, cpi-miR-1677g-3p, cpi-miR-1677 h-3p, cpi-miR-1677 h-5p, cpi-miR-16a-3p, cpi-miR-16a-5p, cpi-miR-16b-3p, cpi-miR-16b-5p, cpi-miR-16c-3p, cpi-miR-16c-5p, cpi-miR-17-3p, cpi-miR-17-5p, cpi-miR-1720-3p, cpi-miR-1729-3p, cpi-miR-1729-5p, cpi-miR-1784-3p, cpi-miR-1788-3p, cpi-miR-1788-5p, cpi-miR-1791-5p, cpi-miR-1805-3p, cpi-miR-1805-5p, cpi-miR-181a-2-3p, cpi-miR-181a-3-3p, cpi-miR-181a-3p, cpi-miR-181a-5p, cpi-miR-181b-1-3p, cpi-miR-181b-3p, cpi-miR-181b-5p, cpi-miR-182-3p, cpi-miR-182-5p, cpi-miR-183-5p, cpi-miR-184-3p, cpi-miR-187-3p, cpi-miR-187-5p, cpi-miR-18a-3p, cpi-miR-18a-5p, cpi-miR-18b-3p, cpi-miR-18b-5p, cpi-miR-190a-5p, cpi-miR-190b-5p, cpi-miR-191-5p, cpi-miR-192-3p, cpi-miR-192-5p, cpi-miR-193a-3p, cpi-miR-193a-5p, cpi-miR-193b-3p, cpi-miR-194-2-3p, cpi-miR-194-3p, cpi-miR-194-5p, cpi-miR-196-2-3p, cpi-miR-196-3p, cpi-miR-196-5p, cpi-miR-199-3p, cpi-miR-199-5p, cpi-miR-19a-3p, cpi-miR-19a-5p, cpi-miR-19b-2-5p, cpi-miR-19b-3p, cpi-miR-19b-5p, cpi-miR-1a-2-5p, cpi-miR-1a-3p, cpi-miR-1a-5p, cpi-miR-1b-3p, cpi-miR-1b-5p, cpi-miR-200a-3p, cpi-miR-200a-5p, cpi-miR-200b-3p, cpi-miR-200b-5p, cpi-miR-202-5p, cpi-miR-203-3p, cpi-miR-203-5p, cpi-miR-204-3-3p, cpi-miR-204-3p, cpi-miR-204-5p, cpi-miR-205a-3p, cpi-miR-205a-5p, cpi-miR-205b-5p, cpi-miR-206-3p, cpi-miR-206-5p, cpi-miR-208-5p, cpi-miR-20a-3p, cpi-miR-20a-5p, cpi-miR-20b-3p, cpi-miR-20b-5p, cpi-miR-21-3p, cpi-miR-21-5p, cpi-miR-210-3p, cpi-miR-210-5p, cpi-miR-212-5p, cpi-miR-214-3p, cpi-miR-214-5p, cpi-miR-215-3p, cpi-miR-215-5p, cpi-miR-216a-5p, cpi-miR-216b-5p, cpi-miR-217-5p, cpi-miR-218-3p, cpi-miR-218-5p, cpi-miR-2184-3p, cpi-miR-2184-5p, cpi-miR-2188-3p, cpi-miR-2188-5p, cpi-miR-219-3p, cpi-miR-219-5p, cpi-miR-22-3p, cpi-miR-22-5p, cpi-miR-221-3p, cpi-miR-221-5p, cpi-miR-222a-3p, cpi-miR-222a-5p, cpi-miR-222b-3p, cpi-miR-222b-5p, cpi-miR-223-3p, cpi-miR-223-5p, cpi-miR-23a-3p, cpi-miR-23b-3p, cpi-miR-24-2-5p, cpi-miR-24-3p, cpi-miR-24-5p, cpi-miR-26-2-3p, cpi-miR-26-3-3p, cpi-miR-26-3p, cpi-miR-26-5p, cpi-miR-27a-3p, cpi-miR-27a-5p, cpi-miR-27b-3p, cpi-miR-27b-5p, cpi-miR-2970-3p, cpi-miR-2970-5p, cpi-miR-2984-3p, cpi-miR-29a-2-5p, cpi-miR-29a-3p, cpi-miR-29a-5p, cpi-miR-29b-2-5p, cpi-miR-29b-3p, cpi-miR-29b-5p, cpi-miR-301a-3p, cpi-miR-301b-3p, cpi-miR-302a-3p, cpi-miR-302b-3p, cpi-miR-302c-3p, cpi-miR-302d-3p, cpi-miR-3064-3p, cpi-miR-30a-3p, cpi-miR-30a-5p, cpi-miR-30b-3p, cpi-miR-30b-5p, cpi-miR-30c-2-3p, cpi-miR-30c-3p, cpi-miR-30c-5p, cpi-miR-30d-3p, cpi-miR-30d-5p, cpi-miR-30e-3p, cpi-miR-30e-5p, cpi-miR-31-3p, cpi-miR-31-5p, cpi-miR-32-3p, cpi-miR-32-5p, cpi-miR-33-2-3p, cpi-miR-33-3p, cpi-miR-33-5p, cpi-miR-338a-3p, cpi-miR-338a-5p, cpi-miR-338b-3p, cpi-miR-338b-5p, cpi-miR-34a-3p, cpi-miR-34a-5p, cpi-miR-34b-3p, cpi-miR-34b-5p, cpi-miR-34c-3p, cpi-miR-34c-5p, cpi-miR-3618-3p, cpi-miR-363-3p, cpi-miR-363-5p, cpi-miR-365-3p, cpi-miR-365-5p, cpi-miR-367-3p, cpi-miR-375-3p, cpi-miR-383-5p, cpi-miR-425-3p, cpi-miR-425-5p, cpi-miR-429-3p, cpi-miR-449a-3p, cpi-miR-449a-5p, cpi-miR-449b-5p, cpi-miR-449c-5p, cpi-miR-449d-5p, cpi-miR-451-3p, cpi-miR-451-5p, cpi-miR-454-3p, cpi-miR-454-5p, cpi-miR-455-3p, cpi-miR-455-5p, cpi-miR-456-3p, cpi-miR-458-3p, cpi-miR-458-5p, cpi-miR-459-3p, cpi-miR-459-5p, cpi-miR-460a-3p, cpi-miR-460a-5p, cpi-miR-460b-5p, cpi-miR-489-3p, cpi-miR-489-5p, cpi-miR-490-3p, cpi-miR-490-5p, cpi-miR-499-3p, cpi-miR-499-5p, cpi-miR-551-2-3p, cpi-miR-551-3p, cpi-miR-551-5p, cpi-miR-599-3p, cpi-miR-726-3p, cpi-miR-727-3p, cpi-miR-737-5p, cpi-miR-7a-2-3p, cpi-miR-7a-3-3p, cpi-miR-7a-3p, cpi-miR-7a-5p, cpi-miR-7b-5p, cpi-miR-875-5p, cpi-miR-9-3p, cpi-miR-9-4-3p, cpi-miR-9-5p, cpi-miR-92a-2-5p, cpi-miR-92a-3p, cpi-miR-92a-5p, cpi-miR-92b-3p, cpi-miR-92b-5p, cpi-miR-92c-3p, cpi-miR-92c-5p, cpi-miR-93-3p, cpi-miR-93-5p, cpi-miR-9578-3p, cpi-miR-9578-5p, cpi-miR-9579-3p, cpi-miR-9579-5p, cpi-miR-9580-3p, cpi-miR-9580-5p, cpi-miR-9581-3p, cpi-miR-9581-5p, cpi-miR-9582-3p, cpi-miR-9582-5p, cpi-miR-9583-3p, cpi-miR-9583-5p, cpi-miR-9584-3p, cpi-miR-9584-5p, cpi-miR-9585-3p, cpi-miR-9585-5p, cpi-miR-9586-3p, cpi-miR-9586-5p, cpi-miR-9587-3p, cpi-miR-9587-5p, cpi-miR-9588-3p, cpi-miR-9588-5p, cpi-miR-9589-3p, cpi-miR-9589-5p, cpi-miR-9590-3p, cpi-miR-9590-5p, cpi-miR-9591-3p, cpi-miR-9591-5p, cpi-miR-9592-3p, cpi-miR-9592-5p, cpi-miR-9593-3p, cpi-miR-9593-5p, cpi-miR-9594-3p, cpi-miR-9594-5p, cpi-miR-9595-3p, cpi-miR-9595-5p, cpi-miR-9596-3p, cpi-miR-9596-5p, cpi-miR-9597-3p, cpi-miR-9597-5p, cpi-miR-96-5p, cpi-miR-98-3p, cpi-miR-98-5p, cpi-miR-99a-3p, cpi-miR-99a-5p, cpi-miR-99b-3p, cpi-miR-99b-5p, cpo-let-7a-2-3p, cpo-let-7a-3p, cpo-let-7a-5p, cpo-let-7b-3p, cpo-let-7b-5p, cpo-let-7c-3p, cpo-let-7c-5p, cpo-let-7d-3p, cpo-let-7d-5p, cpo-let-7e-3p, cpo-let-7e-5p, cpo-let-7f-2-3p, cpo-let-7f-3p, cpo-let-7f-5p, cpo-let-7g-3p, cpo-let-7g-5p, cpo-let-7i-3p, cpo-let-7i-5p, cpo-miR-100-3p, cpo-miR-100-5p, cpo-miR-101-2-5p, cpo-miR-101-3p, cpo-miR-101-5p, cpo-miR-103-2-5p, cpo-miR-103-3p, cpo-miR-103-5p, cpo-miR-105-3p, cpo-miR-105-5p, cpo-miR-106a-3p, cpo-miR-106a-5p, cpo-miR-106b-3p, cpo-miR-106b-5p, cpo-miR-107-3p, cpo-miR-107-5p, cpo-miR-10a-3p, cpo-miR-10a-5p, cpo-miR-10b-3p, cpo-miR-10b-5p, cpo-miR-1185-3p, cpo-miR-1185-5p, cpo-miR-1193-3p, cpo-miR-1193-5p, cpo-miR-1197-3p, cpo-miR-1197-5p, cpo-miR-12072-3p, cpo-miR-12072-5p, cpo-miR-12073-3p, cpo-miR-12073-5p, cpo-miR-12074-3p, cpo-miR-12074-5p, cpo-miR-12075-3p, cpo-miR-12075-5p, cpo-miR-12076-3p, cpo-miR-12076-5p, cpo-miR-12077-3p, cpo-miR-12077-5p, cpo-miR-12078-3p, cpo-miR-12078-5p, cpo-miR-12079-3p, cpo-miR-12079-5p, cpo-miR-12080-3p, cpo-miR-12080-5p, cpo-miR-12081-3p, cpo-miR-12081-5p, cpo-miR-12082-3p, cpo-miR-12082-5p, cpo-miR-12083-3p, cpo-miR-12083-5p, cpo-miR-12084-3p, cpo-miR-12084-5p, cpo-miR-122-3p, cpo-miR-122-5p, cpo-miR-124-3p, cpo-miR-124-5p, cpo-miR-1247-3p, cpo-miR-1247-5p, cpo-miR-1251-3p, cpo-miR-1251-5p, cpo-miR-125a-3p, cpo-miR-125a-5p, cpo-miR-125b-2-3p, cpo-miR-125b-3p, cpo-miR-125b-5p, cpo-miR-126-3p, cpo-miR-126-5p, cpo-miR-1264-3p, cpo-miR-1264-5p, cpo-miR-127-3p, cpo-miR-127-5p, cpo-miR-1271-3p, cpo-miR-1271-5p, cpo-miR-1277-3p, cpo-miR-1277-5p, cpo-miR-128-2-5p, cpo-miR-128-3p, cpo-miR-128-5p, cpo-miR-129-2-3p, cpo-miR-129-3p, cpo-miR-129-5p, cpo-miR-1296-3p, cpo-miR-1296-5p, cpo-miR-1298-3p, cpo-miR-1298-5p, cpo-miR-1301-3p, cpo-miR-1301-5p, cpo-miR-130a-3p, cpo-miR-130a-5p, cpo-miR-130b-3p, cpo-miR-130b-5p, cpo-miR-132-3p, cpo-miR-132-5p, cpo-miR-133a-3p, cpo-miR-133a-5p, cpo-miR-133b-3p, cpo-miR-133b-5p, cpo-miR-134-3p, cpo-miR-134-5p, cpo-miR-1343-3p, cpo-miR-1343-5p, cpo-miR-135a-2-3p, cpo-miR-135a-3p, cpo-miR-135a-5p, cpo-miR-135b-3p, cpo-miR-135b-5p, cpo-miR-136-3p, cpo-miR-136-5p, cpo-miR-1379-3p, cpo-miR-1379-5p, cpo-miR-138-2-3p, cpo-miR-138-3p, cpo-miR-138-5p, cpo-miR-1388-3p, cpo-miR-1388-5p, cpo-miR-139-3p, cpo-miR-139-5p, cpo-miR-140-3p, cpo-miR-140-5p, cpo-miR-141-3p, cpo-miR-141-5p, cpo-miR-142-3p, cpo-miR-142-5p, cpo-miR-143-3p, cpo-miR-143-5p, cpo-miR-144-3p, cpo-miR-144-5p, cpo-miR-145-3p, cpo-miR-145-5p, cpo-miR-146a-3p, cpo-miR-146a-5p, cpo-miR-146b-3p, cpo-miR-146b-5p, cpo-miR-147-3p, cpo-miR-147-5p, cpo-miR-148a-3p, cpo-miR-148a-5p, cpo-miR-148b-3p, cpo-miR-148b-5p, cpo-miR-149-3p, cpo-miR-149-5p, cpo-miR-150-3p, cpo-miR-150-5p, cpo-miR-151-3p, cpo-miR-151-5p, cpo-miR-152-3p, cpo-miR-152-5p, cpo-miR-153-3p, cpo-miR-153-5p, cpo-miR-154-3p, cpo-miR-154-5p, cpo-miR-155-3p, cpo-miR-155-5p, cpo-miR-15a-3p, cpo-miR-15a-5p, cpo-miR-15b-3p, cpo-miR-15b-5p, cpo-miR-16a-3p, cpo-miR-16a-5p, cpo-miR-16b-3p, cpo-miR-16b-5p, cpo-miR-17-3p, cpo-miR-17-5p, cpo-miR-181a-2-3p, cpo-miR-181a-3p, cpo-miR-181a-5p, cpo-miR-181b-2-3p, cpo-miR-181b-3p, cpo-miR-181b-5p, cpo-miR-181c-3p, cpo-miR-181c-5p, cpo-miR-182-3p, cpo-miR-182-5p, cpo-miR-183-3p, cpo-miR-183-5p, cpo-miR-184-3p, cpo-miR-184-5p, cpo-miR-1842-3p, cpo-miR-1842-5p, cpo-miR-185-3p, cpo-miR-185-5p, cpo-miR-186-3p, cpo-miR-186-5p, cpo-miR-187-3p, cpo-miR-187-5p, cpo-miR-188-3p, cpo-miR-188-5p, cpo-miR-18a-3p, cpo-miR-18a-5p, cpo-miR-18b-3p, cpo-miR-18b-5p, cpo-miR-190a-3p, cpo-miR-190a-5p, cpo-miR-190b-3p, cpo-miR-190b-5p, cpo-miR-191-3p, cpo-miR-191-5p, cpo-miR-192-3p, cpo-miR-192-5p, cpo-miR-193a-3p, cpo-miR-193a-5p, cpo-miR-193b-3p, cpo-miR-193b-5p, cpo-miR-194-3p, cpo-miR-194-5p, cpo-miR-195-3p, cpo-miR-195-5p, cpo-miR-196a-3p, cpo-miR-196a-5p, cpo-miR-196b-3p, cpo-miR-196b-5p, cpo-miR-196c-3p, cpo-miR-196c-5p, cpo-miR-197-3p, cpo-miR-197-5p, cpo-miR-199-3-5p, cpo-miR-199-3p, cpo-miR-199-5p, cpo-miR-19a-3p, cpo-miR-19a-5p, cpo-miR-19b-2-5p, cpo-miR-19b-3p, cpo-miR-19b-5p, cpo-miR-1a-3p, cpo-miR-1a-5p, cpo-miR-1b-3p, cpo-miR-1b-5p, cpo-miR-200a-3p, cpo-miR-200a-5p, cpo-miR-200b-3p, cpo-miR-200b-5p, cpo-miR-200c-3p, cpo-miR-200c-5p, cpo-miR-202-3p, cpo-miR-202-5p, cpo-miR-203-3p, cpo-miR-203-5p, cpo-miR-204-3p, cpo-miR-204-5p, cpo-miR-205-3p, cpo-miR-205-5p, cpo-miR-206-3p, cpo-miR-206-5p, cpo-miR-208a-3p, cpo-miR-208a-5p, cpo-miR-208b-3p, cpo-miR-208b-5p, cpo-miR-20a-3p, cpo-miR-20a-5p, cpo-miR-20b-3p, cpo-miR-20b-5p, cpo-miR-21-3p, cpo-miR-21-5p, cpo-miR-210-3p, cpo-miR-210-5p, cpo-miR-211-3p, cpo-miR-211-5p, cpo-miR-212-3p, cpo-miR-212-5p, cpo-miR-214-3p, cpo-miR-214-5p, cpo-miR-215-3p, cpo-miR-215-5p, cpo-miR-216a-3p, cpo-miR-216a-5p, cpo-miR-216b-3p, cpo-miR-216b-5p, cpo-miR-217-3p, cpo-miR-217-5p, cpo-miR-218-2-3p, cpo-miR-218-3p, cpo-miR-218-5p, cpo-miR-219b-3p, cpo-miR-219b-5p, cpo-miR-22-3p, cpo-miR-22-5p, cpo-miR-221-3p, cpo-miR-221-5p, cpo-miR-222-3p, cpo-miR-222-5p, cpo-miR-223-3p, cpo-miR-223-5p, cpo-miR-224-3p, cpo-miR-224-5p, cpo-miR-2355-3p, cpo-miR-2355-5p, cpo-miR-2387-3p, cpo-miR-2387-5p, cpo-miR-23a-3p, cpo-miR-23a-5p, cpo-miR-23b-3p, cpo-miR-23b-5p, cpo-miR-24-2-5p, cpo-miR-24-3p, cpo-miR-24-5p, cpo-miR-25-3p, cpo-miR-25-5p, cpo-miR-26a-2-3p, cpo-miR-26a-3p, cpo-miR-26a-5p, cpo-miR-26b-3p, cpo-miR-26b-5p, cpo-miR-27a-3p, cpo-miR-27a-5p, cpo-miR-27b-3p, cpo-miR-27b-5p, cpo-miR-28-3p, cpo-miR-28-5p, cpo-miR-290-3p, cpo-miR-290-5p, cpo-miR-296-3p, cpo-miR-296-5p, cpo-miR-299-3p, cpo-miR-299-5p, cpo-miR-29a-3p, cpo-miR-29a-5p, cpo-miR-29b-2-5p, cpo-miR-29b-3p, cpo-miR-29b-5p, cpo-miR-29c-3p, cpo-miR-29c-5p, cpo-miR-301a-3p, cpo-miR-301a-5p, cpo-miR-301b-3p, cpo-miR-301b-5p, cpo-miR-302a-3p, cpo-miR-302a-5p, cpo-miR-302b-3p, cpo-miR-302b-5p, cpo-miR-302c-3p, cpo-miR-302c-5p, cpo-miR-302d-3p, cpo-miR-302d-5p, cpo-miR-3059-3p, cpo-miR-3059-5p, cpo-miR-3085-3p, cpo-miR-3085-5p, cpo-miR-30a-3p, cpo-miR-30a-5p, cpo-miR-30b-3p, cpo-miR-30b-5p, cpo-miR-30c-2-3p, cpo-miR-30c-3p, cpo-miR-30c-5p, cpo-miR-30d-3p, cpo-miR-30d-5p, cpo-miR-30e-3p, cpo-miR-30e-5p, cpo-miR-31-3p, cpo-miR-31-5p, cpo-miR-32-3p, cpo-miR-32-5p, cpo-miR-323a-3p, cpo-miR-323a-5p, cpo-miR-323b-3p, cpo-miR-323b-5p, cpo-miR-324-3p, cpo-miR-324-5p, cpo-miR-326-3p, cpo-miR-326-5p, cpo-miR-328-3p, cpo-miR-328-5p, cpo-miR-329-3p, cpo-miR-329-5p, cpo-miR-330-3p, cpo-miR-330-5p, cpo-miR-331-3p, cpo-miR-331-5p, cpo-miR-335-3p, cpo-miR-335-5p, cpo-miR-337-3p, cpo-miR-337-5p, cpo-miR-338-3p, cpo-miR-338-5p, cpo-miR-339-3p, cpo-miR-339-5p, cpo-miR-33a-3p, cpo-miR-33a-5p, cpo-miR-33b-3p, cpo-miR-33b-5p, cpo-miR-340-3p, cpo-miR-340-5p, cpo-miR-342-3p, cpo-miR-342-5p, cpo-miR-345-3p, cpo-miR-345-5p, cpo-miR-346-3p, cpo-miR-346-5p, cpo-miR-34a-3p, cpo-miR-34a-5p, cpo-miR-34b-3p, cpo-miR-34b-5p, cpo-miR-34c-3p, cpo-miR-34c-5p, cpo-miR-350-3p, cpo-miR-350-5p, cpo-miR-361-3p, cpo-miR-361-5p, cpo-miR-3613-3p, cpo-miR-3613-5p, cpo-miR-362-3p, cpo-miR-362-5p, cpo-miR-363-3p, cpo-miR-363-5p, cpo-miR-365-2-5p, cpo-miR-365-3p, cpo-miR-365-5p, cpo-miR-367-3p, cpo-miR-367-5p, cpo-miR-369-3p, cpo-miR-369-5p, cpo-miR-370-3p, cpo-miR-370-5p, cpo-miR-374-3p, cpo-miR-374-5p, cpo-miR-375-3p, cpo-miR-375-5p, cpo-miR-376a-3p, cpo-miR-376a-5p, cpo-miR-376b-3p, cpo-miR-376b-5p, cpo-miR-376c-3p, cpo-miR-376c-5p, cpo-miR-376d-3p, cpo-miR-376d-5p, cpo-miR-377-3p, cpo-miR-377-5p, cpo-miR-378-3p, cpo-miR-378-5p, cpo-miR-379-3p, cpo-miR-379-5p, cpo-miR-380-3p, cpo-miR-380-5p, cpo-miR-381-3p, cpo-miR-381-5p, cpo-miR-382-3p, cpo-miR-382-5p, cpo-miR-383-3p, cpo-miR-383-5p, cpo-miR-3959-3p, cpo-miR-3959-5p, cpo-miR-409-3p, cpo-miR-409-5p, cpo-miR-410-3p, cpo-miR-410-5p, cpo-miR-411-3p, cpo-miR-411-5p, cpo-miR-412-3p, cpo-miR-412-5p, cpo-miR-423-3p, cpo-miR-423-5p, cpo-miR-424-3p, cpo-miR-424-5p, cpo-miR-425-3p, cpo-miR-425-5p, cpo-miR-429-3p, cpo-miR-429-5p, cpo-miR-431-3p, cpo-miR-431-5p, cpo-miR-432-3p, cpo-miR-432-5p, cpo-miR-433-3p, cpo-miR-433-5p, cpo-miR-448-3p, cpo-miR-448-5p, cpo-miR-449a-3p, cpo-miR-449a-5p, cpo-miR-449c-3p, cpo-miR-449c-5p, cpo-miR-450a-2-3p, cpo-miR-450a-3p, cpo-miR-450a-5p, cpo-miR-450b-3p, cpo-miR-450b-5p, cpo-miR-451-5p, cpo-miR-452-3p, cpo-miR-452-5p, cpo-miR-454-3p, cpo-miR-454-5p, cpo-miR-455-3p, cpo-miR-455-5p, cpo-miR-483-3p, cpo-miR-483-5p, cpo-miR-485-3p, cpo-miR-485-5p, cpo-miR-486-3p, cpo-miR-486-5p, cpo-miR-487a-3p, cpo-miR-487a-5p, cpo-miR-487b-3p, cpo-miR-487b-5p, cpo-miR-490-3p, cpo-miR-490-5p, cpo-miR-491-3p, cpo-miR-491-5p, cpo-miR-493-3p, cpo-miR-493-5p, cpo-miR-494-3p, cpo-miR-494-5p, cpo-miR-495-3p, cpo-miR-495-5p, cpo-miR-496-3p, cpo-miR-496-5p, cpo-miR-497-3p, cpo-miR-497-5p, cpo-miR-499-3p, cpo-miR-499-5p, cpo-miR-500a-3p, cpo-miR-500a-5p, cpo-miR-500b-3p, cpo-miR-500b-5p, cpo-miR-501-3p, cpo-miR-501-5p, cpo-miR-502a-3p, cpo-miR-502a-5p, cpo-miR-503-3p, cpo-miR-503-5p, cpo-miR-504-3p, cpo-miR-504-5p, cpo-miR-505-3p, cpo-miR-505-5p, cpo-miR-506-3p, cpo-miR-506-5p, cpo-miR-507a-3p, cpo-miR-507a-5p, cpo-miR-507b-3p, cpo-miR-507b-5p, cpo-miR-507c-3p, cpo-miR-507c-5p, cpo-miR-509a-3p, cpo-miR-509a-5p, cpo-miR-509b-3p, cpo-miR-509b-5p, cpo-miR-509c-3p, cpo-miR-509c-5p, cpo-miR-509d-3p, cpo-miR-509d-5p, cpo-miR-509e-3p, cpo-miR-509e-5p, cpo-miR-509f-3p, cpo-miR-509f-5p, cpo-miR-511-3p, cpo-miR-511-5p, cpo-miR-513-3p, cpo-miR-513-5p, cpo-miR-526-3p, cpo-miR-526-5p, cpo-miR-532-3p, cpo-miR-532-5p, cpo-miR-539-3p, cpo-miR-539-5p, cpo-miR-541-3p, cpo-miR-541-5p, cpo-miR-542-3p, cpo-miR-542-5p, cpo-miR-543-3p, cpo-miR-543-5p, cpo-miR-545-3p, cpo-miR-545-5p, cpo-miR-551-3p, cpo-miR-551-5p, cpo-miR-574-3p, cpo-miR-574-5p, cpo-miR-582-3p, cpo-miR-582-5p, cpo-miR-615-3p, cpo-miR-615-5p, cpo-miR-652-3p, cpo-miR-652-5p, cpo-miR-6529-3p, cpo-miR-6529-5p, cpo-miR-653-3p, cpo-miR-653-5p, cpo-miR-654-3p, cpo-miR-654-5p, cpo-miR-655-3p, cpo-miR-655-5p, cpo-miR-656-3p, cpo-miR-656-5p, cpo-miR-660-3p, cpo-miR-660-5p, cpo-miR-670-3p, cpo-miR-670-5p, cpo-miR-671-3p, cpo-miR-671-5p, cpo-miR-672-3p, cpo-miR-672-5p, cpo-miR-675-3p, cpo-miR-675-5p, cpo-miR-676-3p, cpo-miR-676-5p, cpo-miR-7-2-3p, cpo-miR-7-3-3p, cpo-miR-7-3p, cpo-miR-7-5p, cpo-miR-708-3p, cpo-miR-708-5p, cpo-miR-744-3p, cpo-miR-744-5p, cpo-miR-758-3p, cpo-miR-758-5p, cpo-miR-760-3p, cpo-miR-760-5p, cpo-miR-769-3p, cpo-miR-769-5p, cpo-miR-802-3p, cpo-miR-802-5p, cpo-miR-872-3p, cpo-miR-872-5p, cpo-miR-873-3p, cpo-miR-873-5p, cpo-miR-874-3p, cpo-miR-874-5p, cpo-miR-885-3p, cpo-miR-885-5p, cpo-miR-889-3p, cpo-miR-889-5p, cpo-miR-892-3p, cpo-miR-892-5p, cpo-miR-9-3p, cpo-miR-9-5p, cpo-miR-92a-2-5p, cpo-miR-92a-3p, cpo-miR-92a-5p, cpo-miR-92b-3p, cpo-miR-92b-5p, cpo-miR-93-3p, cpo-miR-93-5p, cpo-miR-95-3p, cpo-miR-95-5p, cpo-miR-96-3p, cpo-miR-96-5p, cpo-miR-98-3p, cpo-miR-98-5p, cpo-miR-99a-3p, cpo-miR-99a-5p, cpo-miR-99b-3p, cpo-miR-99b-5p, cqu-bantam-3p, cqu-bantam-5p, cqu-let-7-3p, cqu-let-7-5p, cqu-miR-1, cqu-miR-10-3p, cqu-miR-10-5p, cqu-miR-100-3p, cqu-miR-100-5p, cqu-miR-1000, cqu-miR-11-3p, cqu-miR-11-5p, cqu-miR-1174, cqu-miR-1175-3p, cqu-miR-1175-5p, cqu-miR-12-3p, cqu-miR-12-5p, cqu-miR-124, cqu-miR-125-3p, cqu-miR-125-5p, cqu-miR-13, cqu-miR-133, cqu-miR-137, cqu-miR-14, cqu-miR-184, cqu-miR-1889-3p, cqu-miR-1889-5p, cqu-miR-1890, cqu-miR-1891, cqu-miR-190, cqu-miR-2, cqu-miR-210-3p, cqu-miR-210-5p, cqu-miR-252-3p, cqu-miR-252-5p, cqu-miR-263, cqu-miR-275, cqu-miR-276-3p, cqu-miR-276-5p, cqu-miR-277, cqu-miR-278, cqu-miR-279-3p, cqu-miR-279-5p, cqu-miR-281-3p, cqu-miR-281-5p, cqu-miR-283, cqu-miR-285-3p, cqu-miR-285-5p, cqu-miR-2941-1-5p, cqu-miR-2941-2-5p, cqu-miR-2941-3p, cqu-miR-2951-3p, cqu-miR-2951-5p, cqu-miR-2952, cqu-miR-305-3p, cqu-miR-305-5p, cqu-miR-306, cqu-miR-308, cqu-miR-309, cqu-miR-31-3p, cqu-miR-31-5p, cqu-miR-315, cqu-miR-316, cqu-miR-317-3p, cqu-miR-317-5p, cqu-miR-33, cqu-miR-375, cqu-miR-7, cqu-miR-71-3p, cqu-miR-71-5p, cqu-miR-79, cqu-miR-8-3p, cqu-miR-8-5p, cqu-miR-87, cqu-miR-9-3p, cqu-miR-9-5p, cqu-miR-92, cqu-miR-932-3p, cqu-miR-932-5p, cqu-miR-957, cqu-miR-965, cqu-miR-970, cqu-miR-980, cqu-miR-981, cqu-miR-988-3p, cqu-miR-988-5p, cqu-miR-989, cqu-miR-993-3p, cqu-miR-993-5p, cqu-miR-996, cqu-miR-998, cqu-miR-999, cqu-miR-iab-4, cre-miR1142, cre-miR1143-3p, cre-miR1143-5p, cre-miR1144a.1, cre-miR1144a.2, cre-miR1144b, cre-miR1145.1, cre-miR1145.2, cre-miR1146, cre-miR1147.1, cre-miR1147.2, cre-miR1148.1, cre-miR1148.2, cre-miR1149.1, cre-miR1149.2, cre-miR1150.1, cre-miR1150.2, cre-miR1150.3, cre-miR1151a-3p, cre-miR1151a-5p, cre-miR1151b-3p, cre-miR1151b-5p, cre-miR1152, cre-miR1153-3p.1, cre-miR1153-3p.2, cre-miR1153-5p.1, cre-miR1153-5p.2, cre-miR1154-3p, cre-miR1154-5p, cre-miR1155, cre-miR1156.1, cre-miR1156.2, cre-miR1157-3p, cre-miR1157-5p, cre-miR1158, cre-miR1159.1, cre-miR1159.2, cre-miR1160.1, cre-miR1160.2, cre-miR1160.3, cre-miR1161a, cre-miR1161b, cre-miR1162-3p, cre-miR1162-5p, cre-miR1163.1, cre-miR1163.2, cre-miR1164, cre-miR1165-3p, cre-miR1165-5p, cre-miR1166.1, cre-miR1166.2, cre-miR1167, cre-miR1168.1, cre-miR1168.2, cre-miR1169-3p, cre-miR1169-5p, cre-miR1170.1, cre-miR1170.2, cre-miR1171, cre-miR1172.1, cre-miR1172.2, cre-miR1173, cre-miR905-3p, cre-miR905-5p, cre-miR906-3p, cre-miR906-5p, cre-miR907, cre-miR908.1, cre-miR908.2, cre-miR908.3, cre-miR909.1, cre-miR909.2, cre-miR909.3, cre-miR910, cre-miR911, cre-miR912, cre-miR913-3p, cre-miR913-5p, cre-miR914, cre-miR915, cre-miR917, cre-miR918, cre-miR919.1, cre-miR919.2, cre-miR9897-3p, cre-miR9897-5p, crm-let-7, crm-lin-4-3p, crm-lin-4-5p, crm-miR-1-3p, crm-miR-1-5p, crm-miR-124a, crm-miR-124b, crm-miR-1817b, crm-miR-1822, crm-miR-1824, crm-miR-2-3p, crm-miR-2-5p, crm-miR-2225, crm-miR-2227, crm-miR-2228, crm-miR-2229, crm-miR-2229a, crm-miR-2229b, crm-miR-2230-3p, crm-miR-2230-5p, crm-miR-2231-3p, crm-miR-2231-5p, crm-miR-2254, crm-miR-228, crm-miR-230-3p, crm-miR-230-5p, crm-miR-231, crm-miR-232, crm-miR-233, crm-miR-234, crm-miR-235-3p, crm-miR-235-5p, crm-miR-236, crm-miR-237-3p, crm-miR-237-5p, crm-miR-238, crm-miR-239a-3p, crm-miR-239a-5p, crm-miR-239b, crm-miR-240, crm-miR-241, crm-miR-242, crm-miR-244-3p, crm-miR-244-5p, crm-miR-245, crm-miR-246-3p, crm-miR-246-5p, crm-miR-247, crm-miR-248, crm-miR-249, crm-miR-250-3p, crm-miR-250-5p, crm-miR-251, crm-miR-252, crm-miR-253, crm-miR-254, crm-miR-255, crm-miR-259, crm-miR-34, crm-miR-355, crm-miR-356, crm-miR-357, crm-miR-358, crm-miR-359, crm-miR-35a, crm-miR-35b, crm-miR-35c-3p, crm-miR-35c-5p, crm-miR-35d, crm-miR-35e, crm-miR-35f, crm-miR-35g, crm-miR-35h, crm-miR-35i, crm-miR-36, crm-miR-360, crm-miR-38, crm-miR-39, crm-miR-392, crm-miR-40, crm-miR-41, crm-miR-42, crm-miR-43, crm-miR-44-3p, crm-miR-44-5p, crm-miR-45-3p, crm-miR-45-5p, crm-miR-46-3p, crm-miR-46-5p, crm-miR-47, crm-miR-48-3p, crm-miR-48-5p, crm-miR-49, crm-miR-50-3p, crm-miR-50-5p, crm-miR-51, crm-miR-52-3p, crm-miR-52-5p, crm-miR-54, crm-miR-55-3p, crm-miR-55-5p, crm-miR-56, crm-miR-57, crm-miR-58a, crm-miR-58b-3p, crm-miR-58b-5p, crm-miR-58c, crm-miR-58d, crm-miR-59, crm-miR-60-3p, crm-miR-60-5p, crm-miR-61, crm-miR-62, crm-miR-63-3p, crm-miR-63-5p, crm-miR-64a-3p, crm-miR-64a-5p, crm-miR-64b-3p, crm-miR-64b-5p, crm-miR-64c-3p, crm-miR-64c-5p, crm-miR-64d-3p, crm-miR-64d-5p, crm-miR-64e, crm-miR-66-3p, crm-miR-66-5p, crm-miR-67-3p, crm-miR-67-5p, crm-miR-70, crm-miR-71-3p, crm-miR-71-5p, crm-miR-72-3p, crm-miR-72-5p, crm-miR-73, crm-miR-74a, crm-miR-74b, crm-miR-75, crm-miR-7579, crm-miR-7582, crm-miR-7594, crm-miR-7597, crm-miR-7598, crm-miR-7599, crm-miR-76, crm-miR-7600, crm-miR-7601, crm-miR-7602, crm-miR-7603, crm-miR-7604, crm-miR-7605, crm-miR-7606, crm-miR-7607, crm-miR-7608, crm-miR-7609, crm-miR-7610, crm-miR-7611, crm-miR-7612, crm-miR-7613, crm-miR-77, crm-miR-784-3p, crm-miR-784-5p, crm-miR-785, crm-miR-786, crm-miR-787, crm-miR-788, crm-miR-79-3p, crm-miR-79-5p, crm-miR-790, crm-miR-80-3p, crm-miR-80-5p, crm-miR-81-3p, crm-miR-81-5p, crm-miR-82, crm-miR-83, crm-miR-84-3p, crm-miR-84-5p, crm-miR-85, crm-miR-86-3p, crm-miR-86-5p, crm-miR-87-3p, crm-miR-87-5p, crm-miR-90, crt-miR166a, crt-miR166b, crt-miR168, crt-miR171, csa-let-7a, csa-let-7b, csa-let-7c, csa-let-7d, csa-miR-124, csa-miR-126, csa-miR-133, csa-miR-141, csa-miR-1473, csa-miR-1497, csa-miR-153, csa-miR-155, csa-miR-183, csa-miR-200, csa-miR-216a, csa-miR-216b, csa-miR-217, csa-miR-219, csa-miR-281, csa-miR-31, csa-miR-34, csa-miR-7, csa-miR-92a, csa-miR-92b, csa-miR-92c, csi-miR12105-3p, csi-miR12105-5p, csi-miR12106-3p, csi-miR12106-5p, csi-miR12107-3p, csi-miR12107-5p, csi-miR12108-3p, csi-miR12108-5p, csi-miR12109-3p, csi-miR12109-5p, csi-miR12110-3p, csi-miR12110-5p, csi-miR12111-3p, csi-miR12111-5p, csi-miR1515a, csi-miR1515b-3p, csi-miR1515b-5p, csi-miR156a-3p, csi-miR156a-5p, csi-miR156b-3p, csi-miR156b-5p, csi-miR156c-3p, csi-miR156c-5p, csi-miR156d-5p, csi-miR156e-3p, csi-miR156e-5p, csi-miR156f-3p, csi-miR156f-5p, csi-miR156g-3p, csi-miR156g-5p, csi-miR156 h-5p, csi-miR156i-5p, csi-miR156j-3p, csi-miR156j-5p, csi-miR159a-3p, csi-miR159a-5p, csi-miR159b-5p, csi-miR159c-3p, csi-miR159c-5p, csi-miR159d, csi-miR160a-3p, csi-miR160a-5p, csi-miR160b-3p, csi-miR160b-5p, csi-miR160c-3p, csi-miR160c-5p, csi-miR162-3p, csi-miR162-5p, csi-miR164a-3p, csi-miR164a-5p, csi-miR164b-3p, csi-miR164b-5p, csi-miR164c-3p, csi-miR164c-5p, csi-miR164d-3p, csi-miR164d-5p, csi-miR166a-3p, csi-miR166a-5p, csi-miR166b-3p, csi-miR166b-5p, csi-miR166c-3p, csi-miR166c-5p, csi-miR166d-3p, csi-miR166d-5p, csi-miR166e-3p, csi-miR166e-5p, csi-miR166f-3p, csi-miR166f-5p, csi-miR166g-3p, csi-miR166g-5p, csi-miR166 h-3p, csi-miR166i-3p, csi-miR166j-3p, csi-miR166k-3p, csi-miR167a-3p, csi-miR167a-5p, csi-miR167b-3p, csi-miR167b-5p, csi-miR167c-3p, csi-miR167c-5p, csi-miR167d-3p, csi-miR167d-5p, csi-miR167e-3p, csi-miR167e-5p, csi-miR168-3p, csi-miR168-5p, csi-miR169a, csi-miR169b-5p, csi-miR169c-3p, csi-miR169c-5p, csi-miR169d-3p, csi-miR169d-5p, csi-miR169e-3p, csi-miR169e-5p, csi-miR169f-5p, csi-miR169g-5p, csi-miR169 h-5p, csi-miR169i-5p, csi-miR169j-5p, csi-miR169k-5p, csi-miR1691-3p, csi-miR1691-5p, csi-miR169m-5p, csi-miR169n-3p, csi-miR169n-5p, csi-miR1690-5p, csi-miR169p-5p, csi-miR169q-5p, csi-miR169r-5p, csi-miR171a, csi-miR171b-3p, csi-miR171b-5p, csi-miR171c-3p, csi-miR171c-5p, csi-miR171d-3p, csi-miR171e-3p, csi-miR171f-3p, csi-miR171f-5p, csi-miR171g-3p, csi-miR171 h-3p, csi-miR171i-3p, csi-miR172a-3p, csi-miR172a-5p, csi-miR172b-3p, csi-miR172b-5p, csi-miR172c-3p, csi-miR172c-5p, csi-miR172d-5p, csi-miR2111-5p, csi-miR2275a-3p, csi-miR2275a-5p, csi-miR2275b-3p, csi-miR2275b-5p, csi-miR3627a-5p, csi-miR3627b-3p, csi-miR3627c-5p, csi-miR390a-3p, csi-miR390a-5p, csi-miR390b-5p, csi-miR391-3p, csi-miR391-5p, csi-miR393a, csi-miR393b-3p, csi-miR393b-5p, csi-miR393c-3p, csi-miR393c-5p, csi-miR3946, csi-miR3947-5p, csi-miR3948, csi-miR3949, csi-miR394a, csi-miR394b-5p, csi-miR3950, csi-miR3951a-3p, csi-miR3951a-5p, csi-miR3951b-3p, csi-miR3951b-5p, csi-miR3952-3p, csi-miR3952-5p, csi-miR3952b-3p, csi-miR3952b-5p, csi-miR3953, csi-miR3954, csi-miR3954b-3p, csi-miR3954b-5p, csi-miR395a, csi-miR395b-3p, csi-miR395b-5p, csi-miR395c-3p, csi-miR396a-3p, csi-miR396a-5p, csi-miR396b-3p, csi-miR396b-5p, csi-miR396c, csi-miR396d-3p, csi-miR396e-3p, csi-miR396e-5p, csi-miR396f-3p, csi-miR396f-5p, csi-miR397-3p, csi-miR397-5p, csi-miR398a-3p, csi-miR398a-5p, csi-miR398b-3p, csi-miR399a-3p, csi-miR399a-5p, csi-miR399b-3p, csi-miR399b-5p, csi-miR399c-3p, csi-miR399c-5p, csi-miR399d-3p, csi-miR399d-5p, csi-miR399e-3p, csi-miR399e-5p, csi-miR399f-3p, csi-miR399f-5p, csi-miR403a-3p, csi-miR403a-5p, csi-miR403b-3p, csi-miR403b-5p, csi-miR408-3p, csi-miR408-5p, csi-miR477a-3p, csi-miR477a-5p, csi-miR477b-3p, csi-miR477b-5p, csi-miR477c-3p, csi-miR477c-5p, csi-miR477d-3p, csi-miR477d-5p, csi-miR477e-3p, csi-miR477e-5p, csi-miR482a-3p, csi-miR482a-5p, csi-miR482b-3p, csi-miR482b-5p, csi-miR482c-3p, csi-miR482c-5p, csi-miR482d-3p, csi-miR482d-5p, csi-miR482e-3p, csi-miR482e-5p, csi-miR482f-3p, csi-miR482f-5p, csi-miR482g-3p, csi-miR482g-5p, csi-miR530a-3p, csi-miR530a-5p, csi-miR530b-3p, csi-miR530b-5p, csi-miR535a, csi-miR535b-3p, csi-miR535b-5p, csi-miR535c-3p, csi-miR535c-5p, csi-miR535d-3p, csi-miR535d-5p, csi-miR535e-5p, csi-miR535f-5p, csi-miR536-3p, csi-miR536-5p, csi-miR827, csi-miR857, csi-miR858-3p, csi-miR9560-3p, csi-miR9560-5p, cst-miR11331, cst-miR11332, cst-miR11333, cst-miR11334, cst-miR11335, cte-bantam, cte-let-7, cte-miR-1, cte-miR-100, cte-miR-10a, cte-miR-10b, cte-miR-10c, cte-miR-10d, cte-miR-1175-3p, cte-miR-1175-5p, cte-miR-12, cte-miR-124, cte-miR-125, cte-miR-133, cte-miR-137, cte-miR-153, cte-miR-184a, cte-miR-184b, cte-miR-190, cte-miR-193, cte-miR-1987, cte-miR-1989, cte-miR-1990a, cte-miR-1990b, cte-miR-1990c-3p, cte-miR-1991, cte-miR-1992, cte-miR-1993, cte-miR-1994, cte-miR-1995, cte-miR-1996a, cte-miR-1996b, cte-miR-1997, cte-miR-1998, cte-miR-1999, cte-miR-2000, cte-miR-2001, cte-miR-210a, cte-miR-210b, cte-miR-216a, cte-miR-216b, cte-miR-216c, cte-miR-219, cte-miR-242, cte-miR-252a, cte-miR-252b, cte-miR-263, cte-miR-2685-3p, cte-miR-2685-5p, cte-miR-2686a-3p, cte-miR-2686a-5p, cte-miR-2686b, cte-miR-2686c, cte-miR-2687, cte-miR-2688-3p, cte-miR-2688-5p, cte-miR-2689, cte-miR-2690, cte-miR-2691-3p, cte-miR-2691-5p, cte-miR-2692, cte-miR-2693, cte-miR-2694, cte-miR-2695, cte-miR-2696, cte-miR-2697, cte-miR-2698, cte-miR-2699-3p, cte-miR-2699-5p, cte-miR-2700, cte-miR-2701, cte-miR-2702-3p, cte-miR-2702-5p, cte-miR-2703-3p, cte-miR-2703-5p, cte-miR-2704, cte-miR-2705, cte-miR-2706, cte-miR-2707, cte-miR-2708, cte-miR-2709, cte-miR-2710, cte-miR-2711, cte-miR-2712, cte-miR-2713, cte-miR-2714, cte-miR-2715, cte-miR-2716, cte-miR-2717, cte-miR-2718, cte-miR-2719, cte-miR-2720-3p, cte-miR-2720-5p, cte-miR-2721, cte-miR-277a, cte-miR-277b, cte-miR-278, cte-miR-279, cte-miR-281, cte-miR-29a, cte-miR-29b, cte-miR-2a, cte-miR-2b, cte-miR-2c, cte-miR-2d, cte-miR-2e, cte-miR-2f, cte-miR-2g, cte-miR-31, cte-miR-315, cte-miR-317, cte-miR-33, cte-miR-34, cte-miR-36, cte-miR-365, cte-miR-375, cte-miR-67, cte-miR-7, cte-miR-71, cte-miR-745a, cte-miR-745b, cte-miR-750, cte-miR-8, cte-miR-87a, cte-miR-87b, cte-miR-9-3p, cte-miR-9-5p, cte-miR-92a, cte-miR-92b, cte-miR-92c, cte-miR-96, cte-miR-981, cte-miR-993, cte-miR-996, ctr-miR156, ctr-miR164, ctr-miR166, ctr-miR167, ctr-miR171, ctr-miR319, dan-bantam, dan-let-7, dan-miR-1, dan-miR-10, dan-miR-100, dan-miR-11, dan-miR-12, dan-miR-124, dan-miR-125, dan-miR-133, dan-miR-13a, dan-miR-13b, dan-miR-14, dan-miR-184-3p, dan-miR-184-5p, dan-miR-210, dan-miR-219, dan-miR-263a, dan-miR-263b, dan-miR-274, dan-miR-275, dan-miR-276a, dan-miR-276b, dan-miR-277, dan-miR-278, dan-miR-279, dan-miR-280, dan-miR-281-1-5p, dan-miR-281-2-5p, dan-miR-281-3p, dan-miR-282, dan-miR-283, dan-miR-284, dan-miR-285, dan-miR-286, dan-miR-287, dan-miR-288, dan-miR-289, dan-miR-2a, dan-miR-2b, dan-miR-2c, dan-miR-3, dan-miR-304, dan-miR-305, dan-miR-306, dan-miR-307, dan-miR-308, dan-miR-309, dan-miR-310, dan-miR-311a, dan-miR-311b, dan-miR-312, dan-miR-314, dan-miR-315, dan-miR-316, dan-miR-317, dan-miR-318, dan-miR-31a, dan-miR-31b, dan-miR-33, dan-miR-34, dan-miR-4, dan-miR-5, dan-miR-6, dan-miR-7, dan-miR-79, dan-miR-8, dan-miR-87, dan-miR-92a, dan-miR-92b, dan-miR-9a, dan-miR-9b, dan-miR-9c, dan-miR-iab-4-3p, dan-miR-iab-4-5p, ddi-miR-1176, ddi-miR-1177, ddi-miR-7095-3p, ddi-miR-7095-5p, ddi-miR-7096, ddi-miR-7097, ddi-miR-7098, ddi-miR-7099, ddi-miR-7100, ddi-miR-7101, ddi-miR-7102, ddi-miR-7103, ddi-miR-7104, ddi-miR-7105, ddi-miR-7131a-3p, ddi-miR-7131a-5p, ddi-miR-7131b, ddi-miR-7131c-3p, ddi-miR-7131c-5p, ddi-miR-7131d, der-bantam, der-let-7, der-miR-1, der-miR-10, der-miR-100, der-miR-11, der-miR-12, der-miR-124, der-miR-125, der-miR-133, der-miR-13a, der-miR-13b, der-miR-14, der-miR-184-3p, der-miR-184-5p, der-miR-210, der-miR-219, der-miR-2498a-3p, der-miR-2498a-5p, der-miR-2498b-3p, der-miR-2498b-5p, der-miR-2499, der-miR-263a, der-miR-263b, der-miR-274, der-miR-275, der-miR-276a, der-miR-276b, der-miR-277, der-miR-278, der-miR-279, der-miR-280, der-miR-281-1-5p, der-miR-281-2-5p, der-miR-281-3p, der-miR-282, der-miR-283, der-miR-284, der-miR-285, der-miR-286, der-miR-287, der-miR-288, der-miR-289, der-miR-2a, der-miR-2b, der-miR-2c, der-miR-3, der-miR-304, der-miR-305, der-miR-306-3p, der-miR-306-5p, der-miR-307, der-miR-308, der-miR-309, der-miR-310a-3p, der-miR-310a-5p, der-miR-310b-3p, der-miR-310b-5p, der-miR-311a-3p, der-miR-311a-5p, der-miR-311b-3p, der-miR-311b-5p, der-miR-311c-3p, der-miR-311c-5p, der-miR-312-3p, der-miR-312-5p, der-miR-314, der-miR-315, der-miR-316, der-miR-317, der-miR-318, der-miR-31a, der-miR-31b, der-miR-33, der-miR-34, der-miR-4, der-miR-5, der-miR-6, der-miR-7, der-miR-79, der-miR-8, der-miR-87, der-miR-92a, der-miR-92b, der-miR-9680-3p, der-miR-9680-5p, der-miR-9681-3p, der-miR-9681-5p, der-miR-9682-3p, der-miR-9682-5p, der-miR-9683-3p, der-miR-9683-5p, der-miR-9691-3p, der-miR-9691-5p, der-miR-973-3p, der-miR-973-5p, der-miR-974-3p, der-miR-974-5p, der-miR-975-3p, der-miR-975-5p, der-miR-976-3p, der-miR-976-5p, der-miR-977-3p, der-miR-977-5p, der-miR-978-3p, der-miR-978-5p, der-miR-983a-3p, der-miR-983a-5p, der-miR-983b-3p, der-miR-983b-5p, der-miR-991-3p, der-miR-991-5p, der-miR-992-??, der-miR-992-2-5p, der-miR-992-5p, der-miR-9a, der-miR-9b, der-miR-9c, der-miR-iab-4-3p, der-miR-iab-4-5p, dev-miR-D1-3p, dev-miR-D1-5p, dev-miR-D10-3p, dev-miR-D11-3p, dev-miR-D12-3p, dev-miR-D12-5p, dev-miR-D13-5p, dev-miR-D14-3p, dev-miR-D15-3p, dev-miR-D16-3p, dev-miR-D17-3p, dev-miR-D17-5p, dev-miR-D18-3p, dev-miR-D18-5p, dev-miR-D19-5p, dev-miR-D2-3p, dev-miR-D20-5p, dev-miR-D21-3p, dev-miR-D21-5p, dev-miR-D22-3p, dev-miR-D22-5p, dev-miR-D23-3p, dev-miR-D24-3p, dev-miR-D3-3p, dev-miR-D4-3p, dev-miR-D5-5p, dev-miR-D6-3p, dev-miR-D6-5p, dev-miR-D7-5p, dev-miR-D8-3p, dev-miR-D8-5p, dev-miR-D9-3p, dev-miR-D9-5p, dgr-bantam, dgr-let-7, dgr-miR-1, dgr-miR-10, dgr-miR-100, dgr-miR-11, dgr-miR-12, dgr-miR-124, dgr-miR-125, dgr-miR-133, dgr-miR-13a, dgr-miR-13b, dgr-miR-14, dgr-miR-184-3p, dgr-miR-184-5p, dgr-miR-210, dgr-miR-219, dgr-miR-263a, dgr-miR-263b, dgr-miR-274, dgr-miR-275, dgr-miR-276a-3p, dgr-miR-276a-5p, dgr-miR-276b-3p, dgr-miR-276b-5p, dgr-miR-277, dgr-miR-278, dgr-miR-279, dgr-miR-280, dgr-miR-281-1-5p, dgr-miR-281-2-5p, dgr-miR-281-3p, dgr-miR-282, dgr-miR-283, dgr-miR-284, dgr-miR-285, dgr-miR-286, dgr-miR-287, dgr-miR-288, dgr-miR-2a, dgr-miR-2b, dgr-miR-2c, dgr-miR-3, dgr-miR-304, dgr-miR-305, dgr-miR-306, dgr-miR-307, dgr-miR-308, dgr-miR-309, dgr-miR-314, dgr-miR-315, dgr-miR-316, dgr-miR-317, dgr-miR-318, dgr-miR-31a, dgr-miR-31b, dgr-miR-33, dgr-miR-34, dgr-miR-4, dgr-miR-5, dgr-miR-6, dgr-miR-7, dgr-miR-79, dgr-miR-8, dgr-miR-87, dgr-miR-92a, dgr-miR-92b, dgr-miR-9a, dgr-miR-9b, dgr-miR-9c, dgr-miR-iab-4-3p, dgr-miR-iab-4-5p, dma-let-7a, dma-let-7b, dma-let-7c, dma-let-7f, dma-let-7g, dma-miR-10, dma-miR-100, dma-miR-101, dma-miR-103a, dma-miR-103b, dma-miR-106b, dma-miR-107, dma-miR-10a, dma-miR-125a, dma-miR-125b, dma-miR-127, dma-miR-1307, dma-miR-130a, dma-miR-1388, dma-miR-140, dma-miR-143, dma-miR-145, dma-miR-148b, dma-miR-151, dma-miR-16, dma-miR-181a, dma-miR-181b, dma-miR-181c, dma-miR-186, dma-miR-191, dma-miR-193, dma-miR-196, dma-miR-199, dma-miR-19b, dma-miR-21, dma-miR-214, dma-miR-22, dma-miR-221, dma-miR-222, dma-miR-23b, dma-miR-24, dma-miR-25, dma-miR-26a, dma-miR-26b, dma-miR-27b, dma-miR-28, dma-miR-296, dma-miR-29a, dma-miR-30a, dma-miR-30c, dma-miR-30e, dma-miR-31, dma-miR-339, dma-miR-340, dma-miR-381, dma-miR-409, dma-miR-423, dma-miR-432, dma-miR-502b, dma-miR-532, dma-miR-6529, dma-miR-660, dma-miR-708, dma-miR-877, dma-miR-92a, dma-miR-93, dma-miR-98, dme-bantam-3p, dme-bantam-5p, dme-let-7-3p, dme-let-7-5p, dme-miR-1-3p, dme-miR-1-5p, dme-miR-10-3p, dme-miR-10-5p, dme-miR-100-3p, dme-miR-100-5p, dme-miR-1000-3p, dme-miR-1000-5p, dme-miR-1001-3p, dme-miR-1001-5p, dme-miR-1002-3p, dme-miR-1002-5p, dme-miR-1003-3p, dme-miR-1003-5p, dme-miR-1004-3p, dme-miR-1004-5p, dme-miR-1005-3p, dme-miR-1006-3p, dme-miR-1006-5p, dme-miR-1007-3p, dme-miR-1007-5p, dme-miR-1008-3p, dme-miR-1008-5p, dme-miR-1009-3p, dme-miR-1010-3p, dme-miR-1010-5p, dme-miR-1011-3p, dme-miR-1012-3p, dme-miR-1012-5p, dme-miR-1013-3p, dme-miR-1014-3p, dme-miR-1014-5p, dme-miR-1015-3p, dme-miR-1016-3p, dme-miR-1016-5p, dme-miR-1017-3p, dme-miR-10404-3p, dme-miR-10404-5p, dme-miR-11-3p, dme-miR-11-5p, dme-miR-11182, dme-miR-12-3p, dme-miR-12-5p, dme-miR-124-3p, dme-miR-124-5p, dme-miR-125-3p, dme-miR-125-5p, dme-miR-133-3p, dme-miR-133-5p, dme-miR-137-3p, dme-miR-137-5p, dme-miR-13a-3p, dme-miR-13a-5p, dme-miR-13b-1-5p, dme-miR-13b-2-5p, dme-miR-13b-3p, dme-miR-14-3p, dme-miR-14-5p, dme-miR-184-3p, dme-miR-184-5p, dme-miR-190-3p, dme-miR-190-5p, dme-miR-193-3p, dme-miR-193-5p, dme-miR-210-3p, dme-miR-210-5p, dme-miR-219-3p, dme-miR-219-5p, dme-miR-2279-3p, dme-miR-2279-5p, dme-miR-2280-3p, dme-miR-2280-5p, dme-miR-2281-3p, dme-miR-2281-5p, dme-miR-2282-3p, dme-miR-2283-3p, dme-miR-2283-5p, dme-miR-2489-3p, dme-miR-2489-5p, dme-miR-2490-3p, dme-miR-2490-5p, dme-miR-2491-3p, dme-miR-2491-5p, dme-miR-2492-3p, dme-miR-2492-5p, dme-miR-2493-3p, dme-miR-2493-5p, dme-miR-2494-3p, dme-miR-2494-5p, dme-miR-2495-3p, dme-miR-2495-5p, dme-miR-2496-3p, dme-miR-2496-5p, dme-miR-2497-3p, dme-miR-2497-5p, dme-miR-2498-3p, dme-miR-2498-5p, dme-miR-2499-3p, dme-miR-2499-5p, dme-miR-2500-3p, dme-miR-2500-5p, dme-miR-2501-3p, dme-miR-2501-5p, dme-miR-252-3p, dme-miR-252-5p, dme-miR-2535b-3p, dme-miR-263a-3p, dme-miR-263a-5p, dme-miR-263b-3p, dme-miR-263b-5p, dme-miR-274-3p, dme-miR-274-5p, dme-miR-275-3p, dme-miR-275-5p, dme-miR-276a-3p, dme-miR-276a-5p, dme-miR-276b-3p, dme-miR-276b-5p, dme-miR-277-3p, dme-miR-277-5p, dme-miR-278-3p, dme-miR-278-5p, dme-miR-279-3p, dme-miR-279-5p, dme-miR-280-5p, dme-miR-281-1-5p, dme-miR-281-2-5p, dme-miR-281-3p, dme-miR-282-3p, dme-miR-282-5p, dme-miR-283-3p, dme-miR-283-5p, dme-miR-284-3p, dme-miR-284-5p, dme-miR-285-3p, dme-miR-285-5p, dme-miR-286-3p, dme-miR-286-5p, dme-miR-287-3p, dme-miR-288-3p, dme-miR-289-5p, dme-miR-2a-1-5p, dme-miR-2a-2-5p, dme-miR-2a-3p, dme-miR-2b-1-5p, dme-miR-2b-2-5p, dme-miR-2b-3p, dme-miR-2c-3p, dme-miR-2c-5p, dme-miR-3-3p, dme-miR-3-5p, dme-miR-303-3p, dme-miR-303-5p, dme-miR-304-3p, dme-miR-304-5p, dme-miR-305-3p, dme-miR-305-5p, dme-miR-306-3p, dme-miR-306-5p, dme-miR-307a-3p, dme-miR-307a-5p, dme-miR-307b-3p, dme-miR-307b-5p, dme-miR-308-3p, dme-miR-308-5p, dme-miR-309-3p, dme-miR-309-5p, dme-miR-310-3p, dme-miR-310-5p, dme-miR-311-3p, dme-miR-311-5p, dme-miR-312-3p, dme-miR-312-5p, dme-miR-313-3p, dme-miR-313-5p, dme-miR-314-3p, dme-miR-314-5p, dme-miR-315-3p, dme-miR-315-5p, dme-miR-316-3p, dme-miR-316-5p, dme-miR-317-3p, dme-miR-317-5p, dme-miR-318-3p, dme-miR-318-5p, dme-miR-31a-3p, dme-miR-31a-5p, dme-miR-31b-3p, dme-miR-31b-5p, dme-miR-33-3p, dme-miR-33-5p, dme-miR-34-3p, dme-miR-34-5p, dme-miR-3641-3p, dme-miR-3641-5p, dme-miR-3642-3p, dme-miR-3642-5p, dme-miR-3643-3p, dme-miR-3643-5p, dme-miR-3644-3p, dme-miR-3644-5p, dme-miR-3645-3p, dme-miR-3645-5p, dme-miR-375-3p, dme-miR-375-5p, dme-miR-4-3p, dme-miR-4-5p, dme-miR-4908-3p, dme-miR-4909-3p, dme-miR-4910-5p, dme-miR-4911-3p, dme-miR-4912-5p, dme-miR-4913-3p, dme-miR-4914-5p, dme-miR-4915-5p, dme-miR-4916-3p, dme-miR-4917-3p, dme-miR-4918-5p, dme-miR-4919-3p, dme-miR-4919-5p, dme-miR-4939-3p, dme-miR-4940-3p, dme-miR-4940-5p, dme-miR-4941-3p, dme-miR-4941-5p, dme-miR-4942-3p, dme-miR-4943-3p, dme-miR-4943-5p, dme-miR-4944-3p, dme-miR-4944-5p, dme-miR-4945-5p, dme-miR-4946-5p, dme-miR-4947-5p, dme-miR-4948-3p, dme-miR-4948-5p, dme-miR-4949-3p, dme-miR-4949-5p, dme-miR-4950-3p, dme-miR-4950-5p, dme-miR-4951-3p, dme-miR-4951-5p, dme-miR-4952-3p, dme-miR-4952-5p, dme-miR-4953-3p, dme-miR-4954-3p, dme-miR-4954-5p, dme-miR-4955-3p, dme-miR-4955-5p, dme-miR-4956-3p, dme-miR-4956-5p, dme-miR-4957-3p, dme-miR-4957-5p, dme-miR-4958-3p, dme-miR-4958-5p, dme-miR-4959-5p, dme-miR-4960-3p, dme-miR-4961-3p, dme-miR-4961-5p, dme-miR-4962-3p, dme-miR-4962-5p, dme-miR-4963-3p, dme-miR-4963-5p, dme-miR-4964-3p, dme-miR-4965-3p, dme-miR-4965-5p, dme-miR-4966-3p, dme-miR-4966-5p, dme-miR-4967-3p, dme-miR-4967-5p, dme-miR-4968-3p, dme-miR-4968-5p, dme-miR-4969-3p, dme-miR-4969-5p, dme-miR-4970-5p, dme-miR-4971-5p, dme-miR-4972-3p, dme-miR-4972-5p, dme-miR-4973-3p, dme-miR-4973-5p, dme-miR-4974-3p, dme-miR-4974-5p, dme-miR-4975-5p, dme-miR-4976-3p, dme-miR-4976-5p, dme-miR-4977-3p, dme-miR-4977-5p, dme-miR-4978-5p, dme-miR-4979-3p, dme-miR-4979-5p, dme-miR-4980-3p, dme-miR-4981-3p, dme-miR-4982-3p, dme-miR-4982-5p, dme-miR-4983-3p, dme-miR-4983-5p, dme-miR-4984-3p, dme-miR-4984-5p, dme-miR-4985-3p, dme-miR-4985-5p, dme-miR-4986-3p, dme-miR-4986-5p, dme-miR-4987-3p, dme-miR-4987-5p, dme-miR-5-3p, dme-miR-5-5p, dme-miR-6-1-5p, dme-miR-6-2-5p, dme-miR-6-3-5p, dme-miR-6-3p, dme-miR-7-3p, dme-miR-7-5p, dme-miR-79-3p, dme-miR-79-5p, dme-miR-8-3p, dme-miR-8-5p, dme-miR-87-3p, dme-miR-87-5p, dme-miR-927-3p, dme-miR-927-5p, dme-miR-929-3p, dme-miR-929-5p, dme-miR-92a-3p, dme-miR-92a-5p, dme-miR-92b-3p, dme-miR-92b-5p, dme-miR-932-3p, dme-miR-932-5p, dme-miR-9369-3p, dme-miR-9369-5p, dme-miR-9370-3p, dme-miR-9370-5p, dme-miR-9371-3p, dme-miR-9371-5p, dme-miR-9372-3p, dme-miR-9372-5p, dme-miR-9373-3p, dme-miR-9373-5p, dme-miR-9374-3p, dme-miR-9374-5p, dme-miR-9375-3p, dme-miR-9375-5p, dme-miR-9376-3p, dme-miR-9376-5p, dme-miR-9377-3p, dme-miR-9377-5p, dme-miR-9378-3p, dme-miR-9378-5p, dme-miR-9379-3p, dme-miR-9379-5p, dme-miR-9380-3p, dme-miR-9380-5p, dme-miR-9381-3p, dme-miR-9381-5p, dme-miR-9382-3p, dme-miR-9382-5p, dme-miR-9383-3p, dme-miR-9383-5p, dme-miR-9384-3p, dme-miR-9384-5p, dme-miR-9385-3p, dme-miR-9385-5p, dme-miR-9388-3p, dme-miR-9388-5p, dme-miR-954-3p, dme-miR-954-5p, dme-miR-955-3p, dme-miR-955-5p, dme-miR-956-3p, dme-miR-956-5p, dme-miR-957-3p, dme-miR-957-5p, dme-miR-958-3p, dme-miR-958-5p, dme-miR-959-3p, dme-miR-959-5p, dme-miR-960-3p, dme-miR-960-5p, dme-miR-961-3p, dme-miR-961-5p, dme-miR-962-3p, dme-miR-962-5p, dme-miR-963-3p, dme-miR-963-5p, dme-miR-964-3p, dme-miR-964-5p, dme-miR-965-3p, dme-miR-965-5p, dme-miR-966-3p, dme-miR-966-5p, dme-miR-967-3p, dme-miR-967-5p, dme-miR-968-3p, dme-miR-968-5p, dme-miR-969-3p, dme-miR-969-5p, dme-miR-970-3p, dme-miR-970-5p, dme-miR-971-3p, dme-miR-971-5p, dme-miR-972-3p, dme-miR-972-5p, dme-miR-973-3p, dme-miR-973-5p, dme-miR-974-3p, dme-miR-974-5p, dme-miR-975-3p, dme-miR-975-5p, dme-miR-976-3p, dme-miR-976-5p, dme-miR-977-3p, dme-miR-977-5p, dme-miR-978-3p, dme-miR-978-5p, dme-miR-979-3p, dme-miR-979-5p, dme-miR-980-3p, dme-miR-980-5p, dme-miR-981-3p, dme-miR-981-5p, dme-miR-982-3p, dme-miR-982-5p, dme-miR-983-3p, dme-miR-983-5p, dme-miR-984-3p, dme-miR-984-5p, dme-miR-985-3p, dme-miR-985-5p, dme-miR-986-3p, dme-miR-986-5p, dme-miR-987-3p, dme-miR-987-5p, dme-miR-988-3p, dme-miR-988-5p, dme-miR-989-3p, dme-miR-989-5p, dme-miR-990-3p, dme-miR-990-5p, dme-miR-991-3p, dme-miR-991-5p, dme-miR-992-3p, dme-miR-992-5p, dme-miR-993-3p, dme-miR-993-5p, dme-miR-994-3p, dme-miR-994-5p, dme-miR-995-3p, dme-miR-995-5p, dme-miR-996-3p, dme-miR-996-5p, dme-miR-997-3p, dme-miR-997-5p, dme-miR-998-3p, dme-miR-998-5p, dme-miR-999-3p, dme-miR-999-5p, dme-miR-9a-3p, dme-miR-9a-5p, dme-miR-9b-3p, dme-miR-9b-5p, dme-miR-9c-3p, dme-miR-9c-5p, dme-miR-iab-4-3p, dme-miR-iab-4-5p, dme-miR-iab-8-3p, dme-miR-iab-8-5p, dmo-bantam, dmo-let-7, dmo-miR-1, dmo-miR-10, dmo-miR-100, dmo-miR-11, dmo-miR-12, dmo-miR-124, dmo-miR-125, dmo-miR-133, dmo-miR-13a, dmo-miR-13b, dmo-miR-14, dmo-miR-184-3p, dmo-miR-184-5p, dmo-miR-210, dmo-miR-219, dmo-miR-263a, dmo-miR-274, dmo-miR-275, dmo-miR-276a, dmo-miR-276b, dmo-miR-277, dmo-miR-278, dmo-miR-279, dmo-miR-280, dmo-miR-281-1-5p, dmo-miR-281-2-5p, dmo-miR-281-3p, dmo-miR-282, dmo-miR-283, dmo-miR-284, dmo-miR-285, dmo-miR-286, dmo-miR-287, dmo-miR-288, dmo-miR-2a, dmo-miR-2b, dmo-miR-2c, dmo-miR-3, dmo-miR-304, dmo-miR-305, dmo-miR-306-3p, dmo-miR-306-5p, dmo-miR-307, dmo-miR-308, dmo-miR-309, dmo-miR-313, dmo-miR-314, dmo-miR-315, dmo-miR-316, dmo-miR-317, dmo-miR-318, dmo-miR-31a, dmo-miR-31b, dmo-miR-33, dmo-miR-34, dmo-miR-4, dmo-miR-5, dmo-miR-6, dmo-miR-7, dmo-miR-79, dmo-miR-8, dmo-miR-87, dmo-miR-92a, dmo-miR-92b, dmo-miR-9a, dmo-miR-9b, dmo-miR-9c, dmo-miR-iab-4-3p, dmo-miR-iab-4-5p, dno-let-7a-3p, dno-let-7a-5p, dno-let-7b-3p, dno-let-7b-5p, dno-let-7c-2-3p, dno-let-7c-3p, dno-let-7c-5p, dno-let-7d-3p, dno-let-7d-5p, dno-let-7f-2-3p, dno-let-7f-3p, dno-let-7f-5p, dno-let-7g-5p, dno-let-7i-3p, dno-let-7i-5p, dno-miR-1-2-5p, dno-miR-1-3p, dno-miR-1-5p, dno-miR-100-3p, dno-miR-100-5p, dno-miR-101-2-5p, dno-miR-101-3p, dno-miR-101-5p, dno-miR-103a-2-5p, dno-miR-103a-3p, dno-miR-103a-5p, dno-miR-106a-3p, dno-miR-106a-5p, dno-miR-106b-3p, dno-miR-106b-5p, dno-miR-107-3p, dno-miR-107-5p, dno-miR-10a-3p, dno-miR-10a-5p, dno-miR-10b-3p, dno-miR-10b-5p, dno-miR-1185-2-3p, dno-miR-1185-3p, dno-miR-1185-5p, dno-miR-1193-3p, dno-miR-1193-5p, dno-miR-1197-3p, dno-miR-1197-5p, dno-miR-12085-3p, dno-miR-12085-5p, dno-miR-12086-3p, dno-miR-12086-5p, dno-miR-12087-3p, dno-miR-12087-5p, dno-miR-122-3p, dno-miR-122-5p, dno-miR-124-3p, dno-miR-124-5p, dno-miR-1249-3p, dno-miR-1249-5p, dno-miR-1251-3p, dno-miR-1251-5p, dno-miR-125b-2-3p, dno-miR-125b-3p, dno-miR-125b-5p, dno-miR-126-3p, dno-miR-126-5p, dno-miR-127-3p, dno-miR-127-5p, dno-miR-1271-3p, dno-miR-1271-5p, dno-miR-1277-3p, dno-miR-1277-5p, dno-miR-128-2-5p, dno-miR-128-3p, dno-miR-128-5p, dno-miR-129-2-3p, dno-miR-129-3p, dno-miR-129-5p, dno-miR-1296-3p, dno-miR-1296-5p, dno-miR-1298-3p, dno-miR-1298-5p, dno-miR-1301-3p, dno-miR-1301-5p, dno-miR-1307-3p, dno-miR-1307-5p, dno-miR-130a-3p, dno-miR-130a-5p, dno-miR-130b-3p, dno-miR-130b-5p, dno-miR-132-3p, dno-miR-132-5p, dno-miR-133a-3p, dno-miR-133a-5p, dno-miR-133b-3p, dno-miR-133b-5p, dno-miR-134-3p, dno-miR-134-5p, dno-miR-1343-3p, dno-miR-1343-5p, dno-miR-135a-2-3p, dno-miR-135a-3p, dno-miR-135a-5p, dno-miR-135b-3p, dno-miR-135b-5p, dno-miR-136-3p, dno-miR-136-5p, dno-miR-137-3p, dno-miR-137-5p, dno-miR-138-2-3p, dno-miR-138-3p, dno-miR-138-5p, dno-miR-1388-3p, dno-miR-1388-5p, dno-miR-139-3p, dno-miR-139-5p, dno-miR-140-3p, dno-miR-140-5p, dno-miR-141-3p, dno-miR-141-5p, dno-miR-142-3p, dno-miR-142-5p, dno-miR-143-3p, dno-miR-143-5p, dno-miR-144-3p, dno-miR-144-5p, dno-miR-146a-3p, dno-miR-146a-5p, dno-miR-146b-3p, dno-miR-146b-5p, dno-miR-147-3p, dno-miR-147-5p, dno-miR-148a-3p, dno-miR-148a-5p, dno-miR-148b-3p, dno-miR-148b-5p, dno-miR-149-3p, dno-miR-149-5p, dno-miR-150-3p, dno-miR-150-5p, dno-miR-151-3p, dno-miR-151-5p, dno-miR-153a-3p, dno-miR-153a-5p, dno-miR-153b-3p, dno-miR-153b-5p, dno-miR-154a-3p, dno-miR-154a-5p, dno-miR-154b-3p, dno-miR-154b-5p, dno-miR-155-3p, dno-miR-155-5p, dno-miR-15a-3p, dno-miR-15a-5p, dno-miR-15b-3p, dno-miR-15b-5p, dno-miR-16a-3p, dno-miR-16a-5p, dno-miR-16b-3p, dno-miR-16b-5p, dno-miR-17-3p, dno-miR-17-5p, dno-miR-181a-2-3p, dno-miR-181a-3p, dno-miR-181a-5p, dno-miR-181b-2-3p, dno-miR-181b-3p, dno-miR-181b-5p, dno-miR-181c-3p, dno-miR-181c-5p, dno-miR-181d-3p, dno-miR-181d-5p, dno-miR-182-3p, dno-miR-182-5p, dno-miR-183-3p, dno-miR-183-5p, dno-miR-184-3p, dno-miR-184-5p, dno-miR-1842-3p, dno-miR-1842-5p, dno-miR-185-3p, dno-miR-185-5p, dno-miR-186-3p, dno-miR-186-5p, dno-miR-187-3p, dno-miR-187-5p, dno-miR-188-3p, dno-miR-188-5p, dno-miR-18a-3p, dno-miR-18a-5p, dno-miR-18b-3p, dno-miR-18b-5p, dno-miR-190a-3p, dno-miR-190a-5p, dno-miR-190b-3p, dno-miR-190b-5p, dno-miR-191-3p, dno-miR-191-5p, dno-miR-192-3p, dno-miR-192-5p, dno-miR-193a-3p, dno-miR-193a-5p, dno-miR-193b-3p, dno-miR-193b-5p, dno-miR-194-3p, dno-miR-194-5p, dno-miR-195-3p, dno-miR-195-5p, dno-miR-196a-3p, dno-miR-196a-5p, dno-miR-196b-3p, dno-miR-196b-5p, dno-miR-196c-3p, dno-miR-196c-5p, dno-miR-197-3p, dno-miR-197-5p, dno-miR-199-2-5p, dno-miR-199-3p, dno-miR-199-5p, dno-miR-19a-3p, dno-miR-19a-5p, dno-miR-19b-2-5p, dno-miR-19b-3p, dno-miR-19b-5p, dno-miR-200b-3p, dno-miR-200b-5p, dno-miR-202-3p, dno-miR-202-5p, dno-miR-204-3p, dno-miR-204-5p, dno-miR-205-3p, dno-miR-205-5p, dno-miR-206-3p, dno-miR-206-5p, dno-miR-208a-3p, dno-miR-208a-5p, dno-miR-208b-3p, dno-miR-208b-5p, dno-miR-20a-3p, dno-miR-20a-5p, dno-miR-20b-3p, dno-miR-20b-5p, dno-miR-21-3p, dno-miR-21-5p, dno-miR-210-3p, dno-miR-210-5p, dno-miR-211-3p, dno-miR-211-5p, dno-miR-2114-3p, dno-miR-2114-5p, dno-miR-214-3p, dno-miR-214-5p, dno-miR-215-3p, dno-miR-215-5p, dno-miR-216a-3p, dno-miR-216a-5p, dno-miR-216b-3p, dno-miR-216b-5p, dno-miR-217-3p, dno-miR-217-5p, dno-miR-218-2-3p, dno-miR-218-3p, dno-miR-218-5p, dno-miR-219a-3p, dno-miR-219a-5p, dno-miR-219b-3p, dno-miR-219b-5p, dno-miR-22-3p, dno-miR-22-5p, dno-miR-221-3p, dno-miR-221-5p, dno-miR-222-3p, dno-miR-222-5p, dno-miR-223-3p, dno-miR-223-5p, dno-miR-224-3p, dno-miR-224-5p, dno-miR-2355-3p, dno-miR-2355-5p, dno-miR-23a-3p, dno-miR-23a-5p, dno-miR-23b-3p, dno-miR-23b-5p, dno-miR-2483-3p, dno-miR-2483-5p, dno-miR-24a-3p, dno-miR-24a-5p, dno-miR-24b-3p, dno-miR-24b-5p, dno-miR-25-3p, dno-miR-25-5p, dno-miR-26a-2-3p, dno-miR-26a-3p, dno-miR-26a-5p, dno-miR-26b-3p, dno-miR-26b-5p, dno-miR-27a-3p, dno-miR-27a-5p, dno-miR-27b-3p, dno-miR-27b-5p, dno-miR-28-3p, dno-miR-28-5p, dno-miR-296-3p, dno-miR-296-5p, dno-miR-299-3p, dno-miR-299-5p, dno-miR-29a-3p, dno-miR-29a-5p, dno-miR-29b-2-5p, dno-miR-29b-3p, dno-miR-29b-5p, dno-miR-29c-3p, dno-miR-29c-5p, dno-miR-301a-3p, dno-miR-301a-5p, dno-miR-301b-3p, dno-miR-301b-5p, dno-miR-302a-3p, dno-miR-302a-5p, dno-miR-302b-3p, dno-miR-302b-5p, dno-miR-302c-3p, dno-miR-302c-5p, dno-miR-302d-3p, dno-miR-302d-5p, dno-miR-3059-3p, dno-miR-3059-5p, dno-miR-3085-3p, dno-miR-3085-5p, dno-miR-30a-3p, dno-miR-30a-5p, dno-miR-30b-3p, dno-miR-30b-5p, dno-miR-30c-2-3p, dno-miR-30c-3p, dno-miR-30c-5p, dno-miR-30d-3p, dno-miR-30d-5p, dno-miR-30e-3p, dno-miR-30e-5p, dno-miR-31-3p, dno-miR-31-5p, dno-miR-32-3p, dno-miR-32-5p, dno-miR-323a-3p, dno-miR-323a-5p, dno-miR-323b-3p, dno-miR-323b-5p, dno-miR-324-3p, dno-miR-324-5p, dno-miR-326-3p, dno-miR-326-5p, dno-miR-328-3p, dno-miR-328-5p, dno-miR-329-3p, dno-miR-329-5p, dno-miR-330-3p, dno-miR-330-5p, dno-miR-331-3p, dno-miR-331-5p, dno-miR-335-3p, dno-miR-335-5p, dno-miR-337-3p, dno-miR-337-5p, dno-miR-338-3p, dno-miR-338-5p, dno-miR-339-3p, dno-miR-339-5p, dno-miR-33a-3p, dno-miR-33a-5p, dno-miR-33b-3p, dno-miR-33b-5p, dno-miR-340-3p, dno-miR-340-5p, dno-miR-342-3p, dno-miR-342-5p, dno-miR-345-3p, dno-miR-345-5p, dno-miR-34a-3p, dno-miR-34a-5p, dno-miR-34b-3p, dno-miR-34b-5p, dno-miR-34c-3p, dno-miR-34c-5p, dno-miR-361-3p, dno-miR-361-5p, dno-miR-3613-3p, dno-miR-3613-5p, dno-miR-362-3p, dno-miR-362-5p, dno-miR-363-3p, dno-miR-363-5p, dno-miR-365-2-5p, dno-miR-365-3p, dno-miR-365-5p, dno-miR-367-3p, dno-miR-367-5p, dno-miR-369-3p, dno-miR-369-5p, dno-miR-371-3p, dno-miR-371-5p, dno-miR-372-3p, dno-miR-372-5p, dno-miR-374a-3p, dno-miR-374a-5p, dno-miR-375-3p, dno-miR-375-5p, dno-miR-376a-3p, dno-miR-376a-5p, dno-miR-376b-3p, dno-miR-376b-5p, dno-miR-376d-3p, dno-miR-376d-5p, dno-miR-377-3p, dno-miR-377-5p, dno-miR-378-3p, dno-miR-378-5p, dno-miR-379-3p, dno-miR-379-5p, dno-miR-380-3p, dno-miR-380-5p, dno-miR-381-3p, dno-miR-381-5p, dno-miR-382-3p, dno-miR-382-5p, dno-miR-383-3p, dno-miR-383-5p, dno-miR-3959-3p, dno-miR-3959-5p, dno-miR-409-3p, dno-miR-409-5p, dno-miR-410-3p, dno-miR-410-5p, dno-miR-411-3p, dno-miR-411-5p, dno-miR-412-3p, dno-miR-412-5p, dno-miR-421-3p, dno-miR-421-5p, dno-miR-423-3p, dno-miR-423-5p, dno-miR-424-3p, dno-miR-424-5p, dno-miR-425-3p, dno-miR-425-5p, dno-miR-429-3p, dno-miR-429-5p, dno-miR-432-3p, dno-miR-432-5p, dno-miR-433-3p, dno-miR-433-5p, dno-miR-449a-3p, dno-miR-449a-5p, dno-miR-449b-3p, dno-miR-449c-3p, dno-miR-449c-5p, dno-miR-450a-3p, dno-miR-450a-5p, dno-miR-450b-3p, dno-miR-450b-5p, dno-miR-450c-3p, dno-miR-450c-5p, dno-miR-451-5p, dno-miR-454-3p, dno-miR-454-5p, dno-miR-455-3p, dno-miR-455-5p, dno-miR-483-3p, dno-miR-483-5p, dno-miR-485-3p, dno-miR-485-5p, dno-miR-486-3p, dno-miR-486-5p, dno-miR-487a-3p, dno-miR-487a-5p, dno-miR-487b-3p, dno-miR-487b-5p, dno-miR-488-3p, dno-miR-488-5p, dno-miR-489-3p, dno-miR-489-5p, dno-miR-490-3p, dno-miR-490-5p, dno-miR-491-3p, dno-miR-491-5p, dno-miR-493-3p, dno-miR-493-5p, dno-miR-494-3p, dno-miR-494-5p, dno-miR-495-3p, dno-miR-495-5p, dno-miR-496-3p, dno-miR-496-5p, dno-miR-497-3p, dno-miR-497-5p, dno-miR-499-3p, dno-miR-499-5p, dno-miR-500a-3p, dno-miR-500a-5p, dno-miR-500b-3p, dno-miR-500b-5p, dno-miR-502-3p, dno-miR-502-5p, dno-miR-503-3p, dno-miR-503-5p, dno-miR-504-3p, dno-miR-504-5p, dno-miR-505-3p, dno-miR-505-5p, dno-miR-506-3p, dno-miR-506-5p, dno-miR-507-3p, dno-miR-507-5p, dno-miR-508-3p, dno-miR-508-5p, dno-miR-509a-2-5p, dno-miR-509a-3p, dno-miR-509a-5p, dno-miR-509b-3p, dno-miR-509b-5p, dno-miR-512-3p, dno-miR-512-5p, dno-miR-532-3p, dno-miR-532-5p, dno-miR-539-3p, dno-miR-539-5p, dno-miR-541-3p, dno-miR-541-5p, dno-miR-542-3p, dno-miR-542-5p, dno-miR-543-3p, dno-miR-543-5p, dno-miR-545-3p, dno-miR-545-5p, dno-miR-551a-3p, dno-miR-551b-3p, dno-miR-551b-5p, dno-miR-574-3p, dno-miR-574-5p, dno-miR-582-3p, dno-miR-582-5p, dno-miR-590-3p, dno-miR-590-5p, dno-miR-628-3p, dno-miR-628-5p, dno-miR-652-3p, dno-miR-652-5p, dno-miR-6529-3p, dno-miR-6529-5p, dno-miR-653-3p, dno-miR-653-5p, dno-miR-654-3p, dno-miR-654-5p, dno-miR-656-3p, dno-miR-656-5p, dno-miR-660-3p, dno-miR-660-5p, dno-miR-670-3p, dno-miR-670-5p, dno-miR-671-3p, dno-miR-671-5p, dno-miR-672-3p, dno-miR-672-5p, dno-miR-675-3p, dno-miR-675-5p, dno-miR-676-3p, dno-miR-676-5p, dno-miR-7-2-3p, dno-miR-7-3-3p, dno-miR-7-3p, dno-miR-7-5p, dno-miR-708-3p, dno-miR-708-5p, dno-miR-744-3p, dno-miR-744-5p, dno-miR-758-3p, dno-miR-758-5p, dno-miR-760-3p, dno-miR-760-5p, dno-miR-802-3p, dno-miR-802-5p, dno-miR-873-3p, dno-miR-873-5p, dno-miR-874-3p, dno-miR-874-5p, dno-miR-885-3p, dno-miR-885-5p, dno-miR-889-3p, dno-miR-889-5p, dno-miR-9-3p, dno-miR-9-5p, dno-miR-92a-2-5p, dno-miR-92a-3p, dno-miR-92a-5p, dno-miR-92b-3p, dno-miR-92b-5p, dno-miR-93-3p, dno-miR-93-5p, dno-miR-95-3p, dno-miR-95-5p, dno-miR-96-3p, dno-miR-96-5p, dno-miR-98-3p, dno-miR-98-5p, dno-miR-99a-3p, dno-miR-99a-5p, dpe-bantam, dpe-let-7, dpe-miR-1, dpe-miR-10, dpe-miR-100, dpe-miR-11, dpe-miR-12, dpe-miR-124, dpe-miR-125, dpe-miR-133, dpe-miR-13a, dpe-miR-13b, dpe-miR-14, dpe-miR-184-3p, dpe-miR-184-5p, dpe-miR-210, dpe-miR-219, dpe-miR-263a, dpe-miR-263b, dpe-miR-274, dpe-miR-275, dpe-miR-276a, dpe-miR-276b, dpe-miR-277, dpe-miR-278, dpe-miR-279, dpe-miR-280, dpe-miR-281-1-5p, dpe-miR-281-2-5p, dpe-miR-281-3p, dpe-miR-282, dpe-miR-283, dpe-miR-284, dpe-miR-286, dpe-miR-287, dpe-miR-288, dpe-miR-289, dpe-miR-2a, dpe-miR-2b, dpe-miR-2c, dpe-miR-3, dpe-miR-305, dpe-miR-306, dpe-miR-307, dpe-miR-308, dpe-miR-309, dpe-miR-314, dpe-miR-315, dpe-miR-316, dpe-miR-317, dpe-miR-318, dpe-miR-31a, dpe-miR-31b, dpe-miR-33, dpe-miR-34, dpe-miR-4, dpe-miR-5, dpe-miR-6, dpe-miR-7, dpe-miR-79, dpe-miR-8, dpe-miR-87, dpe-miR-92a, dpe-miR-92b, dpe-miR-9a, dpe-miR-9b, dpe-miR-9c, dpe-miR-iab-4-3p, dpe-miR-iab-4-5p, dpr-miR156a, dpr-miR156b, dpr-miR160, dpr-miR166a, dpr-miR166b, dpr-miR167a, dpr-miR167b, dpr-miR167c, dpr-miR172a, dpr-miR172b, dpr-miR396, dpr-miR397, dpr-miR408, dps-bantam, dps-let-7, dps-miR-1, dps-miR-10, dps-miR-100, dps-miR-1000-3p, dps-miR-1000-5p, dps-miR-1002-3p, dps-miR-1002-5p, dps-miR-1006-3p, dps-miR-1006-5p, dps-miR-1010-3p, dps-miR-1010-5p, dps-miR-11, dps-miR-12, dps-miR-124, dps-miR-125, dps-miR-133, dps-miR-137-3p, dps-miR-137-5p, dps-miR-13a, dps-miR-13b, dps-miR-14, dps-miR-184, dps-miR-190-3p, dps-miR-190-5p, dps-miR-193-3p, dps-miR-193-5p, dps-miR-210a, dps-miR-210b, dps-miR-219, dps-miR-2502, dps-miR-2503, dps-miR-2504, dps-miR-2505, dps-miR-2506, dps-miR-2507a, dps-miR-2507b-3p, dps-miR-2507b-5p, dps-miR-2508, dps-miR-2509, dps-miR-2510, dps-miR-2511, dps-miR-2512, dps-miR-2513a, dps-miR-2513b, dps-miR-2514, dps-miR-2515, dps-miR-2516, dps-miR-2517-3p, dps-miR-2517-5p, dps-miR-2518, dps-miR-2519, dps-miR-252-3p, dps-miR-252-5p, dps-miR-2520, dps-miR-2521, dps-miR-2522a, dps-miR-2522b-3p, dps-miR-2522b-5p, dps-miR-2523, dps-miR-2524, dps-miR-2525, dps-miR-2526-3p, dps-miR-2527, dps-miR-2528, dps-miR-2529, dps-miR-2530, dps-miR-2531, dps-miR-2532, dps-miR-2533, dps-miR-2534, dps-miR-2535-3p, dps-miR-2535-5p, dps-miR-2536-3p, dps-miR-2536-5p, dps-miR-2537-3p, dps-miR-2537-5p, dps-miR-2538-3p, dps-miR-2538-5p, dps-miR-2539-3p, dps-miR-2539-5p, dps-miR-2540-3p, dps-miR-2540-5p, dps-miR-2541-3p, dps-miR-2541-5p, dps-miR-2542-3p, dps-miR-2542-5p, dps-miR-2543a-3p, dps-miR-2543a-5p, dps-miR-2543b-3p, dps-miR-2543b-5p, dps-miR-2544-3p, dps-miR-2544-5p, dps-miR-2545a-3p, dps-miR-2545a-5p, dps-miR-2545b, dps-miR-2546-3p, dps-miR-2546-5p, dps-miR-2547-3p, dps-miR-2547-5p, dps-miR-2548-3p, dps-miR-2548-5p, dps-miR-2549-3p, dps-miR-2549-5p, dps-miR-2550-3p, dps-miR-2550-5p, dps-miR-2551-3p, dps-miR-2551-5p, dps-miR-2552-3p, dps-miR-2552-5p, dps-miR-2553-3p, dps-miR-2553-5p, dps-miR-2554-3p, dps-miR-2554-5p, dps-miR-2555-3p, dps-miR-2555-5p, dps-miR-2556-3p, dps-miR-2556-5p, dps-miR-2557-3p, dps-miR-2557-5p, dps-miR-2558-3p, dps-miR-2558-5p, dps-miR-2559-3p, dps-miR-2559-5p, dps-miR-2560-3p, dps-miR-2560-5p, dps-miR-2561-3p, dps-miR-2561-5p, dps-miR-2562-3p, dps-miR-2562-5p, dps-miR-2563-3p, dps-miR-2563-5p, dps-miR-2564-3p, dps-miR-2564-5p, dps-miR-2565-3p, dps-miR-2565-5p, dps-miR-2566a-1-5p, dps-miR-2566a-2-5p, dps-miR-2566a-3p, dps-miR-2566b-3p, dps-miR-2566b-5p, dps-miR-2567a-3p, dps-miR-2567a-5p, dps-miR-2567b-3p, dps-miR-2567b-5p, dps-miR-2567c-3p, dps-miR-2567c-5p, dps-miR-2568a-3p, dps-miR-2568a-5p, dps-miR-2568b-3p, dps-miR-2568b-5p, dps-miR-2569-3p, dps-miR-2569-5p, dps-miR-2570-3p, dps-miR-2570-5p, dps-miR-2571-3p, dps-miR-2571-5p, dps-miR-2572-3p, dps-miR-2572-5p, dps-miR-2573-3p, dps-miR-2573-5p, dps-miR-2574a-3p, dps-miR-2574a-5p, dps-miR-2574b, dps-miR-2575-3p, dps-miR-2575-5p, dps-miR-2576, dps-miR-263a, dps-miR-263b, dps-miR-274, dps-miR-275, dps-miR-276a, dps-miR-276b, dps-miR-276c, dps-miR-277, dps-miR-278, dps-miR-279, dps-miR-280, dps-miR-281, dps-miR-282, dps-miR-283, dps-miR-284, dps-miR-285, dps-miR-286, dps-miR-287, dps-miR-288, dps-miR-289, dps-miR-2a, dps-miR-2b, dps-miR-2c, dps-miR-3, dps-miR-304, dps-miR-305, dps-miR-306, dps-miR-307a, dps-miR-307b, dps-miR-308, dps-miR-309, dps-miR-311-3p, dps-miR-311-5p, dps-miR-314, dps-miR-315, dps-miR-316, dps-miR-317, dps-miR-318, dps-miR-31a, dps-miR-31b, dps-miR-33, dps-miR-34, dps-miR-375-3p, dps-miR-375-5p, dps-miR-3983, dps-miR-4, dps-miR-5, dps-miR-6, dps-miR-7, dps-miR-79, dps-miR-8, dps-miR-87, dps-miR-927-3p, dps-miR-927-5p, dps-miR-929-3p, dps-miR-929-5p, dps-miR-92a, dps-miR-92b, dps-miR-92c-3p, dps-miR-92c-5p, dps-miR-932-3p, dps-miR-932-5p, dps-miR-957-3p, dps-miR-957-5p, dps-miR-958-3p, dps-miR-958-5p, dps-miR-959-3p, dps-miR-959-5p, dps-miR-964-3p, dps-miR-964-5p, dps-miR-965-3p, dps-miR-965-5p, dps-miR-968-3p, dps-miR-968-5p, dps-miR-969-3p, dps-miR-969-5p, dps-miR-970-3p, dps-miR-970-5p, dps-miR-971-3p, dps-miR-971-5p, dps-miR-980-3p, dps-miR-980-5p, dps-miR-981-3p, dps-miR-981-5p, dps-miR-986-3p, dps-miR-986-5p, dps-miR-987-3p, dps-miR-987-5p, dps-miR-988-3p, dps-miR-988-5p, dps-miR-989, dps-miR-993-3p, dps-miR-993-5p, dps-miR-994-3p, dps-miR-994-5p, dps-miR-995-3p, dps-miR-995-5p, dps-miR-9a, dps-miR-9b, dps-miR-9c, dps-miR-iab-4-3p, dps-miR-iab-4-5p, dps-miR-iab-8-3p, dps-miR-iab-8-5p, dpu-bantam, dpu-miR-1, dpu-miR-10, dpu-miR-100, dpu-miR-12, dpu-miR-124, dpu-miR-133, dpu-miR-137, dpu-miR-153, dpu-miR-193, dpu-miR-2, dpu-miR-219, dpu-miR-252a, dpu-miR-252b, dpu-miR-263a, dpu-miR-263b, dpu-miR-275, dpu-miR-276, dpu-miR-278, dpu-miR-279a, dpu-miR-279b, dpu-miR-281, dpu-miR-283, dpu-miR-285, dpu-miR-305, dpu-miR-307, dpu-miR-31, dpu-miR-315, dpu-miR-317, dpu-miR-33, dpu-miR-34, dpu-miR-375, dpu-miR-7, dpu-miR-71, dpu-miR-745, dpu-miR-8, dpu-miR-9-3p, dpu-miR-9-5p, dpu-miR-92, dpu-miR-96, dpu-miR-965, dpu-miR-981, dpu-miR-993, dpu-miR-iab-4-3p, dpu-miR-iab-4-5p, dqu-bantam-3p, dqu-bantam-5p, dqu-iab-4-3p, dqu-iab-4-5p, dqu-let-7-3p, dqu-let-7-5p, dqu-miR-10-3p, dqu-miR-10-5p, dqu-miR-100-3p, dqu-miR-100-5p, dqu-miR-1000-3p, dqu-miR-1000-5p, dqu-miR-11-3p, dqu-miR-11-5p, dqu-miR-11651-3p, dqu-miR-11651-5p, dqu-miR-11652-3p, dqu-miR-11652-5p, dqu-miR-11653-3p, dqu-miR-11653-5p, dqu-miR-11654-3p, dqu-miR-11654-5p, dqu-miR-11655-3p, dqu-miR-11655-5p, dqu-miR-11656-3p, dqu-miR-11656-5p, dqu-miR-11657-3p, dqu-miR-11657-5p, dqu-miR-11658-3p, dqu-miR-11658-5p, dqu-miR-11659-3p, dqu-miR-11659-5p, dqu-miR-11660-3p, dqu-miR-11660-5p, dqu-miR-11661-3p, dqu-miR-11661-5p, dqu-miR-11662-3p, dqu-miR-11662-5p, dqu-miR-1175-3p, dqu-miR-1175-5p, dqu-miR-12-3p, dqu-miR-12-5p, dqu-miR-124-3p, dqu-miR-124-5p, dqu-miR-125-3p, dqu-miR-125-5p, dqu-miR-133-3p, dqu-miR-133-5p, dqu-miR-137-3p, dqu-miR-137-5p, dqu-miR-13a-3p, dqu-miR-13a-5p, dqu-miR-13b-3p, dqu-miR-13b-5p, dqu-miR-14-3p, dqu-miR-14-5p, dqu-miR-184-3p, dqu-miR-184-5p, dqu-miR-190-3p, dqu-miR-190-5p, dqu-miR-193-3p, dqu-miR-193-5p, dqu-miR-1a-3p, dqu-miR-1a-5p, dqu-miR-1b-3p, dqu-miR-1b-5p, dqu-miR-210-3p, dqu-miR-210-5p, dqu-miR-219-3p, dqu-miR-219-5p, dqu-miR-252a-3p, dqu-miR-252a-5p, dqu-miR-252b-3p, dqu-miR-252b-5p, dqu-miR-263a-3p, dqu-miR-263a-5p, dqu-miR-263b-3p, dqu-miR-263b-5p, dqu-miR-275-3p, dqu-miR-275-5p, dqu-miR-276-3p, dqu-miR-276-5p, dqu-miR-2765-3p, dqu-miR-2765-5p, dqu-miR-277-3p, dqu-miR-277-5p, dqu-miR-278-3p, dqu-miR-278-5p, dqu-miR-2788-3p, dqu-miR-2788-5p, dqu-miR-2796-3p, dqu-miR-2796-5p, dqu-miR-279a-3p, dqu-miR-279a-5p, dqu-miR-279b-3p, dqu-miR-279b-5p, dqu-miR-279c-3p, dqu-miR-279c-5p, dqu-miR-281-3p, dqu-miR-281-5p, dqu-miR-282-3p, dqu-miR-282-5p, dqu-miR-283-3p, dqu-miR-283-5p, dqu-miR-29-3p, dqu-miR-29-5p, dqu-miR-2a-3p, dqu-miR-2a-5p, dqu-miR-2b-3p, dqu-miR-2b-5p, dqu-miR-305-3p, dqu-miR-305-5p, dqu-miR-306-3p, dqu-miR-306-5p, dqu-miR-307-3p, dqu-miR-307-5p, dqu-miR-31-3p, dqu-miR-31-5p, dqu-miR-315-3p, dqu-miR-315-5p, dqu-miR-316-3p, dqu-miR-316-5p, dqu-miR-317-3p, dqu-miR-317-5p, dqu-miR-33-3p, dqu-miR-33-5p, dqu-miR-34-3p, dqu-miR-34-5p, dqu-miR-3477-3p, dqu-miR-3477-5p, dqu-miR-3715-3p, dqu-miR-3715-5p, dqu-miR-375-3p, dqu-miR-375-5p, dqu-miR-3759-3p, dqu-miR-3770-3p, dqu-miR-3770-5p, dqu-miR-3782-5p, dqu-miR-3783-3p, dqu-miR-3783-5p, dqu-miR-3785-3p, dqu-miR-3785-5p, dqu-miR-3786-3p, dqu-miR-3786-5p, dqu-miR-6001-3p, dqu-miR-6001-5p, dqu-miR-6037-3p, dqu-miR-6037-5p, dqu-miR-6038-3p, dqu-miR-6038-5p, dqu-miR-6039-3p, dqu-miR-6039-5p, dqu-miR-6060-5p, dqu-miR-6065-3p, dqu-miR-6067-5p, dqu-miR-7-3p, dqu-miR-7-5p, dqu-miR-71-3p, dqu-miR-71-5p, dqu-miR-750-3p, dqu-miR-750-5p, dqu-miR-79-3p, dqu-miR-79-5p, dqu-miR-8-3p, dqu-miR-8-5p, dqu-miR-87-3p, dqu-miR-87-5p, dqu-miR-9-3p, dqu-miR-9-5p, dqu-miR-927a-3p, dqu-miR-927a-5p, dqu-miR-927b-3p, dqu-miR-927b-5p, dqu-miR-929-3p, dqu-miR-929-5p, dqu-miR-92a-3p, dqu-miR-92a-5p, dqu-miR-92b-3p, dqu-miR-92b-5p, dqu-miR-92c-3p, dqu-miR-92c-5p, dqu-miR-932-3p, dqu-miR-932-5p, dqu-miR-965-3p, dqu-miR-965-5p, dqu-miR-971-3p, dqu-miR-971-5p, dqu-miR-980-3p, dqu-miR-980-5p, dqu-miR-981-3p, dqu-miR-981-5p, dqu-miR-993-3p, dqu-miR-993-5p, dqu-miR-995-3p, dqu-miR-995-5p, dqu-miR-996-3p, dqu-miR-996-5p, dre-let-7a, dre-let-7b, dre-let-7c-3p, dre-let-7c-5p, dre-let-7d-3p, dre-let-7d-5p, dre-let-7e, dre-let-7f, dre-let-7g, dre-let-7h, dre-let-7i, dre-let-7j, dre-miR-1, dre-miR-100-2-3p, dre-miR-100-3p, dre-miR-100-5p, dre-miR-101a, dre-miR-101b, dre-miR-103, dre-miR-107a-3p, dre-miR-107a-5p, dre-miR-107b, dre-miR-10a-3p, dre-miR-10a-5p, dre-miR-10b-2-3p, dre-miR-10b-3p, dre-miR-10b-5p, dre-miR-10c-3p, dre-miR-10c-5p, dre-miR-10d-3p, dre-miR-10d-5p, dre-miR-122, dre-miR-124-3p, dre-miR-124-4-5p, dre-miR-124-5p, dre-miR-124-6-5p, dre-miR-125a, dre-miR-125b-1-3p, dre-miR-125b-2-3p, dre-miR-125b-3-3p, dre-miR-125b-5p, dre-miR-125c-3p, dre-miR-125c-5p, dre-miR-126a-3p, dre-miR-126a-5p, dre-miR-126b-3p, dre-miR-126b-5p, dre-miR-128-3-5p, dre-miR-128-3p, dre-miR-128-5p, dre-miR-129-1-3p, dre-miR-129-3-3p, dre-miR-129-3p, dre-miR-129-5p, dre-miR-1306, dre-miR-130a, dre-miR-130b, dre-miR-130c-3p, dre-miR-130c-5p, dre-miR-132-3p, dre-miR-132-5p, dre-miR-133a-2-5p, dre-miR-133a-3p, dre-miR-133a-5p, dre-miR-133b-3p, dre-miR-133b-5p, dre-miR-133c-3p, dre-miR-133c-5p, dre-miR-135a, dre-miR-135b-3p, dre-miR-135b-5p, dre-miR-135c, dre-miR-137-3p, dre-miR-137-5p, dre-miR-138-2-3p, dre-miR-138-3p, dre-miR-138-5p, dre-miR-1388-3p, dre-miR-1388-5p, dre-miR-139-3p, dre-miR-139-5p, dre-miR-140-3p, dre-miR-140-5p, dre-miR-141-3p, dre-miR-141-5p, dre-miR-142a-3p, dre-miR-142a-5p, dre-miR-142b-5p, dre-miR-143, dre-miR-144-3p, dre-miR-144-5p, dre-miR-145-3p, dre-miR-145-5p, dre-miR-146a, dre-miR-146b, dre-miR-148, dre-miR-150, dre-miR-152, dre-miR-153a-3p, dre-miR-153a-5p, dre-miR-153b-3p, dre-miR-153b-5p, dre-miR-153c-3p, dre-miR-153c-5p, dre-miR-155, dre-miR-15a-3p, dre-miR-15a-5p, dre-miR-15b-3p, dre-miR-15b-5p, dre-miR-15c, dre-miR-16a, dre-miR-16b, dre-miR-16c-3p, dre-miR-16c-5p, dre-miR-1788-3p, dre-miR-1788-5p, dre-miR-17a-2-3p, dre-miR-17a-3p, dre-miR-17a-5p, dre-miR-181a-2-3p, dre-miR-181a-3-3p, dre-miR-181a-3p, dre-miR-181a-4-3p, dre-miR-181a-5-3p, dre-miR-181a-5p, dre-miR-181b-3-3p, dre-miR-181b-3p, dre-miR-181b-5p, dre-miR-181c-3p, dre-miR-181c-5p, dre-miR-181d-3p, dre-miR-181d-5p, dre-miR-182-3p, dre-miR-182-5p, dre-miR-183-3p, dre-miR-183-5p, dre-miR-184, dre-miR-187, dre-miR-18a, dre-miR-18b-3p, dre-miR-18b-5p, dre-miR-18c, dre-miR-190a, dre-miR-190b, dre-miR-192, dre-miR-193a-3p, dre-miR-193a-5p, dre-miR-193b-3p, dre-miR-193b-5p, dre-miR-194a, dre-miR-194b, dre-miR-196a-3p, dre-miR-196a-5p, dre-miR-196b, dre-miR-196c, dre-miR-196d, dre-miR-199-3-3p, dre-miR-199-3p, dre-miR-199-5p, dre-miR-19a-3p, dre-miR-19a-5p, dre-miR-19b-3p, dre-miR-19b-5p, dre-miR-19c-3p, dre-miR-19c-5p, dre-miR-19d-3p, dre-miR-19d-5p, dre-miR-200a-3p, dre-miR-200a-5p, dre-miR-200b-3p, dre-miR-200b-5p, dre-miR-200c-3p, dre-miR-200c-5p, dre-miR-202-3p, dre-miR-202-5p, dre-miR-203a-3p, dre-miR-203a-5p, dre-miR-203b-3p, dre-miR-203b-5p, dre-miR-204-2-3p, dre-miR-204-3-3p, dre-miR-204-3p, dre-miR-204-5p, dre-miR-205-3p, dre-miR-205-5p, dre-miR-206-3p, dre-miR-206-5p, dre-miR-20a-3p, dre-miR-20a-5p, dre-miR-20b-3p, dre-miR-20b-5p, dre-miR-21, dre-miR-210-3p, dre-miR-210-5p, dre-miR-212, dre-miR-212-3p, dre-miR-212-5p, dre-miR-214, dre-miR-216a, dre-miR-216b, dre-miR-217, dre-miR-2184, dre-miR-2185-3p, dre-miR-2185-5p, dre-miR-2186, dre-miR-2187-3p, dre-miR-2187-5p, dre-miR-2188-3p, dre-miR-2188-5p, dre-miR-2189, dre-miR-218a, dre-miR-218b, dre-miR-219-3p, dre-miR-219-5p, dre-miR-2191, dre-miR-2192, dre-miR-2193, dre-miR-2194, dre-miR-2195, dre-miR-2196, dre-miR-2197, dre-miR-2198, dre-miR-221-3p, dre-miR-221-5p, dre-miR-222a-3p, dre-miR-222a-5p, dre-miR-222b, dre-miR-223, dre-miR-22a-3p, dre-miR-22a-5p, dre-miR-22b-3p, dre-miR-22b-5p, dre-miR-23a-3-5p, dre-miR-23a-3p, dre-miR-23a-5p, dre-miR-23b, dre-miR-23b-3p, dre-miR-23b-5p, dre-miR-24, dre-miR-24b-3p, dre-miR-24b-5p, dre-miR-25-3p, dre-miR-25-5p, dre-miR-26a-2-3p, dre-miR-26a-3p, dre-miR-26a-5p, dre-miR-26b, dre-miR-27a-3p, dre-miR-27a-5p, dre-miR-27b-3p, dre-miR-27b-5p, dre-miR-27c-3p, dre-miR-27c-5p, dre-miR-27d, dre-miR-27e, dre-miR-29a, dre-miR-29b, dre-miR-29b3-3p, dre-miR-29b3-5p, dre-miR-301a, dre-miR-301b-3p, dre-miR-301b-5p, dre-miR-301c-3p, dre-miR-301c-5p, dre-miR-30a-3p, dre-miR-30a-5p, dre-miR-30b, dre-miR-30c-3p, dre-miR-30c-5p, dre-miR-30d, dre-miR-30e-3p, dre-miR-30e-5p, dre-miR-31, dre-miR-338, dre-miR-338-3p, dre-miR-338-5p, dre-miR-34a, dre-miR-34b, dre-miR-34c-3p, dre-miR-34c-5p, dre-miR-363-3p, dre-miR-363-5p, dre-miR-365, dre-miR-375, dre-miR-3906, dre-miR-429a, dre-miR-429b, dre-miR-430a-11-5p, dre-miR-430a-12-5p, dre-miR-430a-13-5p, dre-miR-430a-14-5p, dre-miR-430a-15-5p, dre-miR-430a-16-5p, dre-miR-430a-17-5p, dre-miR-430a-3p, dre-miR-430a-4-5p, dre-miR-430a-5p, dre-miR-430b-3p, dre-miR-430b-5p, dre-miR-430c-3p, dre-miR-430c-5p, dre-miR-430i-3p, dre-miR-430i-5p, dre-miR-451, dre-miR-454a, dre-miR-454b, dre-miR-455-2-5p, dre-miR-455-3p, dre-miR-455-5p, dre-miR-456, dre-miR-457a, dre-miR-457b-3p, dre-miR-457b-5p, dre-miR-458-3p, dre-miR-458-5p, dre-miR-459-3p, dre-miR-459-5p, dre-miR-460-3p, dre-miR-460-5p, dre-miR-461, dre-miR-462, dre-miR-489, dre-miR-499-3p, dre-miR-499-5p, dre-miR-7132-3p, dre-miR-7132-5p, dre-miR-7133-3p, dre-miR-7133-5p, dre-miR-7145, dre-miR-7146-3p, dre-miR-7146-5p, dre-miR-7147, dre-miR-7148-3p, dre-miR-7148-5p, dre-miR-7149-3p, dre-miR-7149-5p, dre-miR-722, dre-miR-723-3p, dre-miR-723-5p, dre-miR-724, dre-miR-725-3p, dre-miR-725-5p, dre-miR-726, dre-miR-727-3p, dre-miR-727-5p, dre-miR-728, dre-miR-729, dre-miR-730, dre-miR-731, dre-miR-732, dre-miR-733, dre-miR-734, dre-miR-735-3p, dre-miR-735-5p, dre-miR-736, dre-miR-737-3p, dre-miR-737-5p, dre-miR-738, dre-miR-740, dre-miR-7a, dre-miR-7b, dre-miR-9-3p, dre-miR-9-4-3p, dre-miR-9-5p, dre-miR-9-7-3p, dre-miR-92a-2-5p, dre-miR-92a-3p, dre-miR-92a-5p, dre-miR-92b-3p, dre-miR-92b-5p, dre-miR-93, dre-miR-96-3p, dre-miR-96-5p, dre-miR-99, dse-bantam, dse-let-7, dse-miR-1, dse-miR-10, dse-miR-100, dse-miR-11, dse-miR-12, dse-miR-124, dse-miR-125, dse-miR-133, dse-miR-13a, dse-miR-13b, dse-miR-14, dse-miR-184-3p, dse-miR-184-5p, dse-miR-210, dse-miR-219, dse-miR-2498a-3p, dse-miR-2498a-5p, dse-miR-2498b-3p, dse-miR-2498b-5p, dse-miR-2499-3p, dse-miR-2499-5p, dse-miR-2582-3p, dse-miR-2582-5p, dse-miR-263a, dse-miR-263b, dse-miR-274, dse-miR-275, dse-miR-276a, dse-miR-276b, dse-miR-277, dse-miR-278, dse-miR-279, dse-miR-280, dse-miR-281-1-5p, dse-miR-281-2-5p, dse-miR-281-3p, dse-miR-282, dse-miR-283, dse-miR-284, dse-miR-285, dse-miR-286, dse-miR-287, dse-miR-288, dse-miR-289, dse-miR-2a, dse-miR-2b, dse-miR-2c, dse-miR-3, dse-miR-303-3p, dse-miR-303-5p, dse-miR-304, dse-miR-305, dse-miR-306-3p, dse-miR-306-5p, dse-miR-307, dse-miR-308, dse-miR-309, dse-miR-310-3p, dse-miR-310-5p, dse-miR-311-3p, dse-miR-311-5p, dse-miR-312-3p, dse-miR-312-5p, dse-miR-313-3p, dse-miR-313-5p, dse-miR-314, dse-miR-315, dse-miR-316, dse-miR-317, dse-miR-318, dse-miR-31a, dse-miR-31b, dse-miR-33, dse-miR-34, dse-miR-4, dse-miR-4966-2-3p, dse-miR-4966-3p, dse-miR-4966-5p, dse-miR-5, dse-miR-6, dse-miR-7, dse-miR-79, dse-miR-8, dse-miR-87, dse-miR-92a, dse-miR-92b, dse-miR-9369-3p, dse-miR-9369-5p, dse-miR-9680-3p, dse-miR-9680-5p, dse-miR-9685-3p, dse-miR-9685-5p, dse-miR-973-3p, dse-miR-973-5p, dse-miR-974-3p, dse-miR-974-5p, dse-miR-975-3p, dse-miR-975-5p, dse-miR-976, dse-miR-977-3p, dse-miR-977-5p, dse-miR-978a-3p, dse-miR-978a-5p, dse-miR-978b, dse-miR-982b, dse-miR-982c-3p, dse-miR-982c-5p, dse-miR-983-3p, dse-miR-983-5p, dse-miR-991-3p, dse-miR-991-5p, dse-miR-992-3p, dse-miR-992-5p, dse-miR-9a, dse-miR-9b, dse-miR-9c, dse-miR-iab-4-3p, dse-miR-iab-4-5p, dsi-bantam, dsi-let-7, dsi-miR-1, dsi-miR-10, dsi-miR-100, dsi-miR-1000-3p, dsi-miR-1000-5p, dsi-miR-1001-3p, dsi-miR-1001-5p, dsi-miR-1002-3p, dsi-miR-1002-5p, dsi-miR-1003-3p, dsi-miR-1003-5p, dsi-miR-1005-3p, dsi-miR-1005-5p, dsi-miR-1006-3p, dsi-miR-1006-5p, dsi-miR-1010-3p, dsi-miR-1010-5p, dsi-miR-1011, dsi-miR-1012-3p, dsi-miR-1012-5p, dsi-miR-1013, dsi-miR-11, dsi-miR-12, dsi-miR-124, dsi-miR-125, dsi-miR-133, dsi-miR-137-3p, dsi-miR-137-5p, dsi-miR-13a, dsi-miR-13b, dsi-miR-14, dsi-miR-184-3p, dsi-miR-184-5p, dsi-miR-190-3p, dsi-miR-190-5p, dsi-miR-193-3p, dsi-miR-193-5p, dsi-miR-210, dsi-miR-219, dsi-miR-2489, dsi-miR-2494-3p, dsi-miR-2494-5p, dsi-miR-2498a-3p, dsi-miR-2498a-5p, dsi-miR-2498b-3p, dsi-miR-2498b-5p, dsi-miR-2498c-3p, dsi-miR-2498c-5p, dsi-miR-2499-3p, dsi-miR-2499-5p, dsi-miR-252-3p, dsi-miR-252-5p, dsi-miR-2577, dsi-miR-2578, dsi-miR-2579, dsi-miR-2580, dsi-miR-2581, dsi-miR-2582a-3p, dsi-miR-2582a-5p, dsi-miR-2582b-3p, dsi-miR-2582b-5p, dsi-miR-2583-3p, dsi-miR-2583-5p, dsi-miR-2584-3p, dsi-miR-2584-5p, dsi-miR-263a, dsi-miR-263b, dsi-miR-275, dsi-miR-276a, dsi-miR-276b, dsi-miR-277, dsi-miR-278, dsi-miR-279, dsi-miR-281-3p, dsi-miR-281-5p, dsi-miR-282, dsi-miR-283, dsi-miR-284, dsi-miR-285, dsi-miR-286, dsi-miR-287, dsi-miR-288, dsi-miR-289, dsi-miR-2a, dsi-miR-2b, dsi-miR-2c, dsi-miR-3, dsi-miR-303-3p, dsi-miR-303-5p, dsi-miR-304, dsi-miR-305, dsi-miR-307, dsi-miR-308, dsi-miR-309, dsi-miR-310-3p, dsi-miR-310-5p, dsi-miR-311-3p, dsi-miR-311-5p, dsi-miR-312-3p, dsi-miR-312-5p, dsi-miR-313-3p, dsi-miR-313-5p, dsi-miR-314, dsi-miR-315, dsi-miR-316, dsi-miR-317, dsi-miR-318, dsi-miR-31a, dsi-miR-31b, dsi-miR-33, dsi-miR-34, dsi-miR-375-3p, dsi-miR-375-5p, dsi-miR-4, dsi-miR-4966-3-3p, dsi-miR-4966-3p, dsi-miR-4966-5p, dsi-miR-5, dsi-miR-6-3p, dsi-miR-6-5p, dsi-miR-7, dsi-miR-8, dsi-miR-87, dsi-miR-927-3p, dsi-miR-927-5p, dsi-miR-929-3p, dsi-miR-929-5p, dsi-miR-92a, dsi-miR-92b, dsi-miR-932-3p, dsi-miR-932-5p, dsi-miR-9369-3p, dsi-miR-9369-5p, dsi-miR-955-3p, dsi-miR-955-5p, dsi-miR-957, dsi-miR-958-3p, dsi-miR-958-5p, dsi-miR-959, dsi-miR-960, dsi-miR-961-3p, dsi-miR-961-5p, dsi-miR-962, dsi-miR-963-3p, dsi-miR-963-5p, dsi-miR-964-3p, dsi-miR-964-5p, dsi-miR-965-3p, dsi-miR-965-5p, dsi-miR-967-3p, dsi-miR-967-5p, dsi-miR-968-3p, dsi-miR-968-5p, dsi-miR-9680-3p, dsi-miR-9680-5p, dsi-miR-969-3p, dsi-miR-969-5p, dsi-miR-970-3p, dsi-miR-970-5p, dsi-miR-972-3p, dsi-miR-972-5p, dsi-miR-973-3p, dsi-miR-973-5p, dsi-miR-974-3p, dsi-miR-974-5p, dsi-miR-975-3p, dsi-miR-975-5p, dsi-miR-976-3p, dsi-miR-976-5p, dsi-miR-977-3p, dsi-miR-977-5p, dsi-miR-978a-3p, dsi-miR-978a-5p, dsi-miR-978b-3p, dsi-miR-978b-5p, dsi-miR-980-3p, dsi-miR-980-5p, dsi-miR-981-3p, dsi-miR-981-5p, dsi-miR-982a-3p, dsi-miR-982a-5p, dsi-miR-982b-3p, dsi-miR-982b-5p, dsi-miR-982c-3p, dsi-miR-982c-5p, dsi-miR-983a-3p, dsi-miR-983a-5p, dsi-miR-983b-3p, dsi-miR-983b-5p, dsi-miR-986-3p, dsi-miR-986-5p, dsi-miR-987-3p, dsi-miR-987-5p, dsi-miR-988-3p, dsi-miR-988-5p, dsi-miR-989, dsi-miR-991-3p, dsi-miR-991-5p, dsi-miR-992-3p, dsi-miR-992-5p, dsi-miR-993-3p, dsi-miR-993-5p, dsi-miR-995-3p, dsi-miR-995-5p, dsi-miR-999-3p, dsi-miR-999-5p, dsi-miR-9a, dsi-miR-iab-4-3p, dsi-miR-iab-4-5p, dsi-miR-iab-8-3p, dsi-miR-iab-8-5p, dvi-bantam-3p, dvi-bantam-5p, dvi-let-7, dvi-miR-1-3p, dvi-miR-1-5p, dvi-miR-10-3p, dvi-miR-10-5p, dvi-miR-100-3p, dvi-miR-100-5p, dvi-miR-1000-3p, dvi-miR-1000-5p, dvi-miR-1002-3p, dvi-miR-1002-5p, dvi-miR-1003-3p, dvi-miR-1006-3p, dvi-miR-1006-5p, dvi-miR-1007-3p, dvi-miR-1010-3p, dvi-miR-1010-5p, dvi-miR-1017-3p, dvi-miR-11-3p, dvi-miR-11-5p, dvi-miR-12-3p, dvi-miR-12-5p, dvi-miR-124-3p, dvi-miR-124-5p, dvi-miR-125-3p, dvi-miR-125-5p, dvi-miR-133-3p, dvi-miR-133-5p, dvi-miR-137-3p, dvi-miR-13a-3p, dvi-miR-13a-5p, dvi-miR-13b-2-5p, dvi-miR-13b-3p, dvi-miR-13b-5p, dvi-miR-14-3p, dvi-miR-14-5p, dvi-miR-184-3p, dvi-miR-184-5p, dvi-miR-190-3p, dvi-miR-190-5p, dvi-miR-193-3p, dvi-miR-193-5p, dvi-miR-210-3p, dvi-miR-210-5p, dvi-miR-219-3p, dvi-miR-219-5p, dvi-miR-252-3p, dvi-miR-252-5p, dvi-miR-263a-3p, dvi-miR-263a-5p, dvi-miR-263b-3p, dvi-miR-263b-5p, dvi-miR-274, dvi-miR-275-3p, dvi-miR-275-5p, dvi-miR-276a-3p, dvi-miR-276a-5p, dvi-miR-276b-3p, dvi-miR-276b-5p, dvi-miR-277-3p, dvi-miR-277-5p, dvi-miR-278-3p, dvi-miR-278-5p, dvi-miR-279-3p, dvi-miR-279-5p, dvi-miR-280, dvi-miR-281-1-5p, dvi-miR-281-2-5p, dvi-miR-281-3p, dvi-miR-282-3p, dvi-miR-282-5p, dvi-miR-283-3p, dvi-miR-283-5p, dvi-miR-284-3p, dvi-miR-284-5p, dvi-miR-285-3p, dvi-miR-285-5p, dvi-miR-286-3p, dvi-miR-286-5p, dvi-miR-287, dvi-miR-288, dvi-miR-2a-2-5p, dvi-miR-2a-3p, dvi-miR-2a-5p, dvi-miR-2b-3p, dvi-miR-2b-5p, dvi-miR-2c-3p, dvi-miR-2c-5p, dvi-miR-3-3p, dvi-miR-3-5p, dvi-miR-304-3p, dvi-miR-304-5p, dvi-miR-305-3p, dvi-miR-305-5p, dvi-miR-306-3p, dvi-miR-306-5p, dvi-miR-307, dvi-miR-308-3p, dvi-miR-308-5p, dvi-miR-309-3p, dvi-miR-309-5p, dvi-miR-310-3p, dvi-miR-310-5p, dvi-miR-311-3p, dvi-miR-311-5p, dvi-miR-313-3p, dvi-miR-313-5p, dvi-miR-314-3p, dvi-miR-314-5p, dvi-miR-315a, dvi-miR-315b, dvi-miR-316-3p, dvi-miR-316-5p, dvi-miR-317-3p, dvi-miR-317-5p, dvi-miR-318, dvi-miR-31a-3p, dvi-miR-31a-5p, dvi-miR-31b, dvi-miR-33-3p, dvi-miR-33-5p, dvi-miR-34-3p, dvi-miR-34-5p, dvi-miR-375-3p, dvi-miR-375-5p, dvi-miR-4-3p, dvi-miR-4-5p, dvi-miR-5-3p, dvi-miR-5-5p, dvi-miR-6-1-5p, dvi-miR-6-2-5p, dvi-miR-6-3-5p, dvi-miR-6-3p, dvi-miR-7-3p, dvi-miR-7-5p, dvi-miR-79-3p, dvi-miR-79-5p, dvi-miR-8-3p, dvi-miR-8-5p, dvi-miR-87-3p, dvi-miR-87-5p, dvi-miR-927-3p, dvi-miR-927-5p, dvi-miR-929-5p, dvi-miR-92a-3p, dvi-miR-92a-5p, dvi-miR-92b-3p, dvi-miR-92b-5p, dvi-miR-932-3p, dvi-miR-932-5p, dvi-miR-9538-3p, dvi-miR-9538-5p, dvi-miR-9538b-3p, dvi-miR-9538b-5p, dvi-miR-9539-3p, dvi-miR-9539-5p, dvi-miR-9540-3p, dvi-miR-9540-5p, dvi-miR-9541a-3p, dvi-miR-9541a-5p, dvi-miR-9541b-3p, dvi-miR-9541b-5p, dvi-miR-9542a-3p, dvi-miR-9542a-5p, dvi-miR-9542b-3p, dvi-miR-9542b-5p, dvi-miR-9542c-3p, dvi-miR-9542c-5p, dvi-miR-9542d-3p, dvi-miR-9542d-5p, dvi-miR-9542e-3p, dvi-miR-9542e-5p, dvi-miR-9543a-3p, dvi-miR-9543a-5p, dvi-miR-9544a-3p, dvi-miR-9544a-5p, dvi-miR-9544b-3p, dvi-miR-9544b-5p, dvi-miR-9544c-3p, dvi-miR-9544c-5p, dvi-miR-9545-3p, dvi-miR-9545-5p, dvi-miR-9546-3p, dvi-miR-9546-5p, dvi-miR-9547-3p, dvi-miR-9547-5p, dvi-miR-9548-3p, dvi-miR-9548-5p, dvi-miR-9549-3p, dvi-miR-9549-5p, dvi-miR-955-3p, dvi-miR-9550-3p, dvi-miR-9550-5p, dvi-miR-956-3p, dvi-miR-956-5p, dvi-miR-957-3p, dvi-miR-958-3p, dvi-miR-958-5p, dvi-miR-962-5p, dvi-miR-963-5p, dvi-miR-964-5p, dvi-miR-965-3p, dvi-miR-965-5p, dvi-miR-968-3p, dvi-miR-968-5p, dvi-miR-969-3p, dvi-miR-969-5p, dvi-miR-9692-3p, dvi-miR-9692-5p, dvi-miR-9693-3p, dvi-miR-9693-5p, dvi-miR-9694-3p, dvi-miR-9694-5p, dvi-miR-9695-3p, dvi-miR-9695-5p, dvi-miR-9696a-3p, dvi-miR-9696a-5p, dvi-miR-9696b-3p, dvi-miR-9696b-5p, dvi-miR-9697-3p, dvi-miR-9697-5p, dvi-miR-9698-3p, dvi-miR-9698-5p, dvi-miR-9699-3p, dvi-miR-9699-5p, dvi-miR-970-3p, dvi-miR-970-5p, dvi-miR-9700a-3p, dvi-miR-9700a-5p, dvi-miR-9700b-3p, dvi-miR-9700b-5p, dvi-miR-9701a-3p, dvi-miR-9701a-5p, dvi-miR-9701b-3p, dvi-miR-9701b-5p, dvi-miR-9702-3p, dvi-miR-9702-5p, dvi-miR-9703-3p, dvi-miR-9703-5p, dvi-miR-9704-2-3p, dvi-miR-9704-3p, dvi-miR-9704-5p, dvi-miR-9705-3p, dvi-miR-9705-5p, dvi-miR-9706a-3p, dvi-miR-9706a-5p, dvi-miR-9706b-3p, dvi-miR-9706b-5p, dvi-miR-9707-3p, dvi-miR-9707-5p, dvi-miR-9708a-3p, dvi-miR-9708a-5p, dvi-miR-9708b-3p, dvi-miR-9708b-5p, dvi-miR-9708c-3p, dvi-miR-9708c-5p, dvi-miR-9708d-3p, dvi-miR-9708d-5p, dvi-miR-9709-3p, dvi-miR-9709-5p, dvi-miR-971-3p, dvi-miR-9710-3p, dvi-miR-9710-5p, dvi-miR-9711-3p, dvi-miR-9711-5p, dvi-miR-9712-3p, dvi-miR-9712-5p, dvi-miR-9713-3p, dvi-miR-9713-5p, dvi-miR-9714-3p, dvi-miR-9714-5p, dvi-miR-9716-3p, dvi-miR-9716-5p, dvi-miR-9717-3p, dvi-miR-9717-5p, dvi-miR-9719-3p, dvi-miR-9719-5p, dvi-miR-9720-3p, dvi-miR-9720-5p, dvi-miR-9721-3p, dvi-miR-9721-5p, dvi-miR-973-3p, dvi-miR-973-5p, dvi-miR-974-1-3p, dvi-miR-974-1-5p, dvi-miR-974-2-5p, dvi-miR-974-3p, dvi-miR-975-3p, dvi-miR-975-5p, dvi-miR-976-3p, dvi-miR-976-5p, dvi-miR-977-3p, dvi-miR-977-5p, dvi-miR-980-3p, dvi-miR-980-5p, dvi-miR-981-3p, dvi-miR-981-5p, dvi-miR-986-3p, dvi-miR-986-5p, dvi-miR-987-5p, dvi-miR-988-3p, dvi-miR-988-5p, dvi-miR-989-3p, dvi-miR-989-5p, dvi-miR-993-3p, dvi-miR-993-5p, dvi-miR-994-3p, dvi-miR-994-5p, dvi-miR-995-3p, dvi-miR-995-5p, dvi-miR-996-3p, dvi-miR-996-5p, dvi-miR-998-3p, dvi-miR-998-5p, dvi-miR-999-3p, dvi-miR-999-5p, dvi-miR-9a-3p, dvi-miR-9a-5p, dvi-miR-9b-3p, dvi-miR-9b-5p, dvi-miR-9c-3p, dvi-miR-9c-5p, dvi-miR-iab-4-3p, dvi-miR-iab-4-5p, dvi-miR-iab-8-3p, dvi-miR-iab-8-5p, dwi-bantam, dwi-let-7, dwi-miR-1, dwi-miR-10, dwi-miR-100, dwi-miR-11, dwi-miR-12, dwi-miR-124, dwi-miR-125, dwi-miR-133, dwi-miR-13a, dwi-miR-13b, dwi-miR-14, dwi-miR-184-3p, dwi-miR-184-5p, dwi-miR-210, dwi-miR-219, dwi-miR-263a, dwi-miR-263b, dwi-miR-274, dwi-miR-275, dwi-miR-276a-3p, dwi-miR-276a-5p, dwi-miR-276b-3p, dwi-miR-276b-5p, dwi-miR-277, dwi-miR-278, dwi-miR-279, dwi-miR-280, dwi-miR-281-1-5p, dwi-miR-281-2-5p, dwi-miR-281-3p, dwi-miR-282, dwi-miR-283, dwi-miR-284, dwi-miR-285, dwi-miR-286, dwi-miR-287, dwi-miR-288, dwi-miR-2a, dwi-miR-2b, dwi-miR-2c, dwi-miR-3, dwi-miR-304, dwi-miR-305, dwi-miR-306, dwi-miR-307, dwi-miR-308, dwi-miR-309, dwi-miR-314, dwi-miR-315, dwi-miR-316, dwi-miR-317, dwi-miR-318, dwi-miR-31a, dwi-miR-31b, dwi-miR-33, dwi-miR-34, dwi-miR-4, dwi-miR-5, dwi-miR-6, dwi-miR-7, dwi-miR-79, dwi-miR-8, dwi-miR-87, dwi-miR-92a, dwi-miR-92b, dwi-miR-9a, dwi-miR-9b, dwi-miR-9c, dwi-miR-iab-4-3p, dwi-miR-iab-4-5p, dya-bantam, dya-let-7, dya-miR-1, dya-miR-10, dya-miR-100, dya-miR-11, dya-miR-124, dya-miR-125, dya-miR-133, dya-miR-13a, dya-miR-13b, dya-miR-14, dya-miR-184-3p, dya-miR-184-5p, dya-miR-210, dya-miR-219, dya-miR-2498-3p, dya-miR-2498-5p, dya-miR-2499, dya-miR-263a, dya-miR-263b, dya-miR-274, dya-miR-275, dya-miR-276a, dya-miR-276b, dya-miR-277, dya-miR-278, dya-miR-279, dya-miR-280, dya-miR-281-1-5p, dya-miR-281-2-5p, dya-miR-281-3p, dya-miR-282, dya-miR-283, dya-miR-284, dya-miR-285, dya-miR-286, dya-miR-287, dya-miR-288, dya-miR-289, dya-miR-2a, dya-miR-2b, dya-miR-2c, dya-miR-3, dya-miR-303, dya-miR-304, dya-miR-305, dya-miR-306-3p, dya-miR-306-5p, dya-miR-307, dya-miR-308, dya-miR-309, dya-miR-310-3p, dya-miR-310-5p, dya-miR-311a-3p, dya-miR-311a-5p, dya-miR-311b-3p, dya-miR-311b-5p, dya-miR-312-3p, dya-miR-312-5p, dya-miR-314, dya-miR-315, dya-miR-316, dya-miR-317, dya-miR-318, dya-miR-31a, dya-miR-31b, dya-miR-33, dya-miR-34, dya-miR-4, dya-miR-5, dya-miR-6, dya-miR-7, dya-miR-79, dya-miR-8, dya-miR-87, dya-miR-92a, dya-miR-92b, dya-miR-9681-3p, dya-miR-9681-5p, dya-miR-9682-3p, dya-miR-9682-5p, dya-miR-9683-3p, dya-miR-9683-5p, dya-miR-9690-3p, dya-miR-9690-5p, dya-miR-975-3p, dya-miR-975-5p, dya-miR-976-3p, dya-miR-976-5p, dya-miR-977-3p, dya-miR-977-5p, dya-miR-978-3p, dya-miR-978-5p, dya-miR-991-3p, dya-miR-991-5p, dya-miR-992-3p, dya-miR-992-5p, dya-miR-9a, dya-miR-9b, dya-miR-9c, dya-miR-iab-4-3p, dya-miR-iab-4-5p, ebv-miR-BART1-3p, ebv-miR-BART1-5p, ebv-miR-BART10-3p, ebv-miR-BART10-5p, ebv-miR-BART11-3p, ebv-miR-BART11-5p, ebv-miR-BART12, ebv-miR-BART13-3p, ebv-miR-BART13-5p, ebv-miR-BART14-3p, ebv-miR-BART14-5p, ebv-miR-BART15, ebv-miR-BART16, ebv-miR-BART17-3p, ebv-miR-BART17-5p, ebv-miR-BART18-3p, ebv-miR-BART18-5p, ebv-miR-BART19-3p, ebv-miR-BART19-5p, ebv-miR-BART2-3p, ebv-miR-BART2-5p, ebv-miR-BART20-3p, ebv-miR-BART20-5p, ebv-miR-BART21-3p, ebv-miR-BART21-5p, ebv-miR-BART22, ebv-miR-BART3-3p, ebv-miR-BART3-5p, ebv-miR-BART4-3p, ebv-miR-BART4-5p, ebv-miR-BART5-3p, ebv-miR-BART5-5p, ebv-miR-BART6-3p, ebv-miR-BART6-5p, ebv-miR-BART7-3p, ebv-miR-BART7-5p, ebv-miR-BART8-3p, ebv-miR-BART8-5p, ebv-miR-BART9-3p, ebv-miR-BART9-5p, ebv-miR-BHRF1-1, ebv-miR-BHRF1-2-3p, ebv-miR-BHRF1-2-5p, ebv-miR-BHRF1-3, eca-let-7a, eca-let-7c, eca-let-7d, eca-let-7e, eca-let-7f, eca-let-7g, eca-miR-1, eca-miR-100, eca-miR-101, eca-miR-103, eca-miR-105, eca-miR-106a, eca-miR-106b, eca-miR-107a, eca-miR-107b, eca-miR-10a, eca-miR-10b, eca-miR-1179, eca-miR-1180, eca-miR-1185, eca-miR-1193, eca-miR-1197, eca-miR-1204, eca-miR-122, eca-miR-124, eca-miR-1244, eca-miR-1248, eca-miR-1249, eca-miR-1255b, eca-miR-125a-3p, eca-miR-125a-5p, eca-miR-125b-5p, eca-miR-126-3p, eca-miR-126-5p, eca-miR-1261, eca-miR-1264, eca-miR-127, eca-miR-1271a, eca-miR-1271b, eca-miR-128, eca-miR-1282, eca-miR-1289, eca-miR-1291a, eca-miR-1291b, eca-miR-1296, eca-miR-1298, eca-miR-129a-3p, eca-miR-129a-5p, eca-miR-129b-3p, eca-miR-129b-5p, eca-miR-1301, eca-miR-1302, eca-miR-1302b, eca-miR-1302c, eca-miR-1302d, eca-miR-1302e, eca-miR-1307, eca-miR-130a, eca-miR-130b, eca-miR-132, eca-miR-133a, eca-miR-133b, eca-miR-134, eca-miR-135a, eca-miR-135b, eca-miR-136, eca-miR-137, eca-miR-1379, eca-miR-138, eca-miR-1388, eca-miR-139-3p, eca-miR-139-5p, eca-miR-140-3p, eca-miR-140-5p, eca-miR-141, eca-miR-142-3p, eca-miR-142-5p, eca-miR-143, eca-miR-144, eca-miR-145, eca-miR-1461, eca-miR-1468, eca-miR-146a, eca-miR-146b-3p, eca-miR-146b-5p, eca-miR-147b, eca-miR-148a, eca-miR-148b-3p, eca-miR-148b-5p, eca-miR-149, eca-miR-150, eca-miR-151-5p, eca-miR-153, eca-miR-1543, eca-miR-154a, eca-miR-154b, eca-miR-155, eca-miR-1597, eca-miR-15a, eca-miR-15b, eca-miR-16, eca-miR-17, eca-miR-181a, eca-miR-181b, eca-miR-182, eca-miR-183, eca-miR-1839, eca-miR-184, eca-miR-1842, eca-miR-185, eca-miR-186, eca-miR-187, eca-miR-188-3p, eca-miR-188-5p, eca-miR-1892, eca-miR-1898, eca-miR-18a, eca-miR-18b, eca-miR-1902, eca-miR-1905a, eca-miR-1905b, eca-miR-1905c, eca-miR-190a, eca-miR-190b, eca-miR-1911, eca-miR-1912, eca-miR-191a, eca-miR-191b, eca-miR-192, eca-miR-193a-3p, eca-miR-193a-5p, eca-miR-193b, eca-miR-194, eca-miR-195, eca-miR-196a, eca-miR-196b, eca-miR-197, eca-miR-199a-3p, eca-miR-199a-5p, eca-miR-199b-3p, eca-miR-199b-5p, eca-miR-19a, eca-miR-19b, eca-miR-200a, eca-miR-200b, eca-miR-200c, eca-miR-204b, eca-miR-205, eca-miR-206, eca-miR-208a, eca-miR-208b, eca-miR-20a, eca-miR-20b, eca-miR-21, eca-miR-211, eca-miR-2114, eca-miR-212, eca-miR-214, eca-miR-215, eca-miR-216a, eca-miR-216b, eca-miR-217, eca-miR-218, eca-miR-219-5p, eca-miR-22, eca-miR-221, eca-miR-222, eca-miR-223, eca-miR-224, eca-miR-23a, eca-miR-23b, eca-miR-24, eca-miR-2483, eca-miR-25, eca-miR-26a, eca-miR-27a, eca-miR-27b, eca-miR-28-3p, eca-miR-28-5p, eca-miR-296, eca-miR-299, eca-miR-29a, eca-miR-29b, eca-miR-29c, eca-miR-301a, eca-miR-301b-3p, eca-miR-301b-5p, eca-miR-302a, eca-miR-302b, eca-miR-302c, eca-miR-302d, eca-miR-3065, eca-miR-30b, eca-miR-30c, eca-miR-30d, eca-miR-30e, eca-miR-31, eca-miR-32, eca-miR-3200, eca-miR-323-3p, eca-miR-323-5p, eca-miR-324-3p, eca-miR-324-5p, eca-miR-326, eca-miR-328, eca-miR-329a, eca-miR-329b, eca-miR-330, eca-miR-331, eca-miR-335, eca-miR-337-3p, eca-miR-337-5p, eca-miR-338-3p, eca-miR-338-5p, eca-miR-33a, eca-miR-33b, eca-miR-340-3p, eca-miR-340-5p, eca-miR-342-3p, eca-miR-342-5p, eca-miR-345-3p, eca-miR-345-5p, eca-miR-346, eca-miR-34a, eca-miR-34b-3p, eca-miR-34b-5p, eca-miR-34c, eca-miR-350, eca-miR-3548, eca-miR-361-3p, eca-miR-361-5p, eca-miR-3613, eca-miR-362-3p, eca-miR-362-5p, eca-miR-363, eca-miR-365, eca-miR-367, eca-miR-369-3p, eca-miR-369-5p, eca-miR-370, eca-miR-371-3p, eca-miR-371-5p, eca-miR-372, eca-miR-374a, eca-miR-374b, eca-miR-376a, eca-miR-376b, eca-miR-376c, eca-miR-377, eca-miR-378, eca-miR-379, eca-miR-380, eca-miR-381, eca-miR-382, eca-miR-383, eca-miR-384, eca-miR-3958, eca-miR-3959, eca-miR-409-3p, eca-miR-409-5p, eca-miR-410, eca-miR-411, eca-miR-412, eca-miR-421, eca-miR-423-3p, eca-miR-423-5p, eca-miR-424, eca-miR-425, eca-miR-429, eca-miR-431, eca-miR-432, eca-miR-433, eca-miR-448, eca-miR-449a, eca-miR-449b, eca-miR-450a, eca-miR-450b-3p, eca-miR-450b-5p, eca-miR-450c, eca-miR-451, eca-miR-454, eca-miR-483, eca-miR-485-3p, eca-miR-485-5p, eca-miR-486-3p, eca-miR-486-5p, eca-miR-487a, eca-miR-487b, eca-miR-488, eca-miR-489, eca-miR-490-3p, eca-miR-490-5p, eca-miR-491-3p, eca-miR-491-5p, eca-miR-492, eca-miR-493a, eca-miR-493b, eca-miR-494, eca-miR-495, eca-miR-496, eca-miR-497, eca-miR-499-3p, eca-miR-499-5p, eca-miR-500, eca-miR-501-5p, eca-miR-502-3p, eca-miR-502-5p, eca-miR-503, eca-miR-504, eca-miR-505, eca-miR-506a, eca-miR-507a, eca-miR-507b, eca-miR-508-3p, eca-miR-508-5p, eca-miR-509a-5p, eca-miR-509b, eca-miR-514, eca-miR-514a, eca-miR-514b, eca-miR-532-3p, eca-miR-532-5p, eca-miR-539, eca-miR-541, eca-miR-542-3p, eca-miR-542-5p, eca-miR-543, eca-miR-544, eca-miR-544b, eca-miR-545, eca-miR-551a, eca-miR-551b, eca-miR-568, eca-miR-582-3p, eca-miR-582-5p, eca-miR-590-3p, eca-miR-590-5p, eca-miR-592, eca-miR-598, eca-miR-615-3p, eca-miR-615-5p, eca-miR-628a, eca-miR-632, eca-miR-652, eca-miR-6529, eca-miR-653, eca-miR-655, eca-miR-656, eca-miR-660, eca-miR-664, eca-miR-670, eca-miR-671-3p, eca-miR-671-5p, eca-miR-672, eca-miR-675, eca-miR-676, eca-miR-684, eca-miR-7, eca-miR-703, eca-miR-7035, eca-miR-7045, eca-miR-708, eca-miR-711, eca-miR-7143, eca-miR-7177a, eca-miR-7177b, eca-miR-744, eca-miR-758, eca-miR-761, eca-miR-763, eca-miR-764-3p, eca-miR-764-5p, eca-miR-7667, eca-miR-767-3p, eca-miR-767-5p, eca-miR-769a-3p, eca-miR-769a-5p, eca-miR-769b, eca-miR-770-5p, eca-miR-802, eca-miR-872, eca-miR-873, eca-miR-874, eca-miR-876-3p, eca-miR-876-5p, eca-miR-885-3p, eca-miR-885-5p, eca-miR-889, eca-miR-8908a, eca-miR-8908b, eca-miR-8908c, eca-miR-8908d, eca-miR-8908e, eca-miR-8908f, eca-miR-8908g, eca-miR-8908h, eca-miR-8908i, eca-miR-8908j, eca-miR-8908k, eca-miR-89081, eca-miR-8908m, eca-miR-8908n, eca-miR-8909, eca-miR-8910, eca-miR-8911, eca-miR-8912, eca-miR-8913, eca-miR-8914, eca-miR-8915, eca-miR-8916, eca-miR-8917, eca-miR-8918, eca-miR-8919, eca-miR-8920, eca-miR-8921, eca-miR-8922, eca-miR-8923, eca-miR-8924, eca-miR-8925, eca-miR-8926, eca-miR-8927, eca-miR-8928, eca-miR-8929, eca-miR-8930, eca-miR-8931, eca-miR-8932, eca-miR-8933, eca-miR-8934, eca-miR-8935, eca-miR-8936, eca-miR-8937, eca-miR-8938, eca-miR-8939, eca-miR-8940, eca-miR-8941, eca-miR-8942, eca-miR-8943, eca-miR-8944, eca-miR-8945, eca-miR-8946, eca-miR-8947, eca-miR-8948, eca-miR-8949, eca-miR-8950, eca-miR-8951, eca-miR-8952, eca-miR-8953, eca-miR-8954, eca-miR-8955, eca-miR-8956, eca-miR-8957, eca-miR-8958, eca-miR-8959, eca-miR-8960, eca-miR-8961, eca-miR-8962, eca-miR-8963, eca-miR-8964, eca-miR-8965, eca-miR-8966, eca-miR-8967, eca-miR-8968, eca-miR-8969, eca-miR-8970, eca-miR-8971, eca-miR-8972, eca-miR-8973, eca-miR-8974, eca-miR-8975, eca-miR-8976, eca-miR-8977, eca-miR-8978, eca-miR-8979, eca-miR-8980, eca-miR-8981, eca-miR-8982, eca-miR-8983, eca-miR-8984, eca-miR-8985, eca-miR-8986a, eca-miR-8986b, eca-miR-8987, eca-miR-8988, eca-miR-8989, eca-miR-8990, eca-miR-8991, eca-miR-8992, eca-miR-8993, eca-miR-8994, eca-miR-8995, eca-miR-8996, eca-miR-8997, eca-miR-8998, eca-miR-8999, eca-miR-9000, eca-miR-9001, eca-miR-9002, eca-miR-9003, eca-miR-9004, eca-miR-9005, eca-miR-9006, eca-miR-9007, eca-miR-9008, eca-miR-9009, eca-miR-9010, eca-miR-9011, eca-miR-9012, eca-miR-9013, eca-miR-9014, eca-miR-9015, eca-miR-9016, eca-miR-9017, eca-miR-9018, eca-miR-9019, eca-miR-9020, eca-miR-9021, eca-miR-9022, eca-miR-9023, eca-miR-9024, eca-miR-9025, eca-miR-9026, eca-miR-9027, eca-miR-9028, eca-miR-9029, eca-miR-9030, eca-miR-9031, eca-miR-9032, eca-miR-9033, eca-miR-9034, eca-miR-9035, eca-miR-9036, eca-miR-9037, eca-miR-9038, eca-miR-9039, eca-miR-9040, eca-miR-9041, eca-miR-9042, eca-miR-9043, eca-miR-9044, eca-miR-9045, eca-miR-9046, eca-miR-9047, eca-miR-9048, eca-miR-9049, eca-miR-9050, eca-miR-9051, eca-miR-9052, eca-miR-9053, eca-miR-9054, eca-miR-9055, eca-miR-9056, eca-miR-9057, eca-miR-9058a, eca-miR-9058b, eca-miR-9059, eca-miR-9060, eca-miR-9061, eca-miR-9062, eca-miR-9063, eca-miR-9064, eca-miR-9065, eca-miR-9066, eca-miR-9067, eca-miR-9068, eca-miR-9069, eca-miR-9070, eca-miR-9071, eca-miR-9072, eca-miR-9073, eca-miR-9074, eca-miR-9075, eca-miR-9076, eca-miR-9077, eca-miR-9078, eca-miR-9079, eca-miR-9080, eca-miR-9081, eca-miR-9082, eca-miR-9083, eca-miR-9084, eca-miR-9085, eca-miR-9086, eca-miR-9087, eca-miR-9088, eca-miR-9089, eca-miR-9090, eca-miR-9091, eca-miR-9092, eca-miR-9093, eca-miR-9094, eca-miR-9095, eca-miR-9096, eca-miR-9097, eca-miR-9098, eca-miR-9099, eca-miR-9100, eca-miR-9101, eca-miR-9102, eca-miR-9103, eca-miR-9104, eca-miR-9105, eca-miR-9106, eca-miR-9107, eca-miR-9108, eca-miR-9109, eca-miR-9110, eca-miR-9111, eca-miR-9112, eca-miR-9113, eca-miR-9114, eca-miR-9115, eca-miR-9116, eca-miR-9117, eca-miR-9118, eca-miR-9119, eca-miR-9120, eca-miR-9121, eca-miR-9122, eca-miR-9123, eca-miR-9124, eca-miR-9125, eca-miR-9126, eca-miR-9127, eca-miR-9128, eca-miR-9129, eca-miR-9130, eca-miR-9131, eca-miR-9132, eca-miR-9133, eca-miR-9134, eca-miR-9135, eca-miR-9136, eca-miR-9137, eca-miR-9138, eca-miR-9139, eca-miR-9140, eca-miR-9141, eca-miR-9142, eca-miR-9143, eca-miR-9144, eca-miR-9145, eca-miR-9146, eca-miR-9147, eca-miR-9148, eca-miR-9149, eca-miR-9150, eca-miR-9151, eca-miR-9152, eca-miR-9153, eca-miR-9154, eca-miR-9155, eca-miR-9156, eca-miR-9157, eca-miR-9158, eca-miR-9159, eca-miR-9160, eca-miR-9161, eca-miR-9162, eca-miR-9163, eca-miR-9164, eca-miR-9165, eca-miR-9166, eca-miR-9167, eca-miR-9168, eca-miR-9169, eca-miR-9170, eca-miR-9171, eca-miR-9172, eca-miR-9173, eca-miR-9174, eca-miR-9175, eca-miR-9176, eca-miR-9177, eca-miR-9178, eca-miR-9179, eca-miR-9180, eca-miR-9181, eca-miR-9182, eca-miR-9183, eca-miR-9184, eca-miR-9185, eca-miR-92a, eca-miR-92b, eca-miR-93, eca-miR-95, eca-miR-96, eca-miR-98, eca-miR-99a, eca-miR-99b, eca-miR-9a, eel-miR-11050-3p, eel-miR-11050-5p, eel-miR-11051-5p, eel-miR-11052-3p, eel-miR-11053-3p, eel-miR-11053-5p, eel-miR-11054-3p, eel-miR-11054-5p, eel-miR-11055-3p, eel-miR-11055-5p, eel-miR-11056-3p, eel-miR-11056-5p, eel-miR-11057-3p, eel-miR-11057-5p, eel-miR-11058-5p, eel-miR-11059-3p, eel-miR-11059-5p, eel-miR-11060-3p, eel-miR-11060-5p, eel-miR-11061-3p, eel-miR-11062-3p, eel-miR-11062-5p, eel-miR-11063-3p, eel-miR-11063-5p, eel-miR-11064-3p, eel-miR-11065-3p, eel-miR-11065-5p, eel-miR-7132-3p, eel-miR-7132-5p, eel-miR-7552-2-3p, eel-miR-7552-3p, eel-miR-7552-5p, eel-miR-8160-3p, eel-miR-8160-5p, efu-let-7c, efu-let-7d, efu-let-7e, efu-let-7f, efu-miR-1, efu-miR-10, efu-miR-101, efu-miR-103a, efu-miR-103b, efu-miR-106, efu-miR-107, efu-miR-1185, efu-miR-1193, efu-miR-124, efu-miR-1249, efu-miR-125a, efu-miR-125b, efu-miR-126, efu-miR-127, efu-miR-1271, efu-miR-128a, efu-miR-128b, efu-miR-129a, efu-miR-129b, efu-miR-130, efu-miR-1307, efu-miR-132, efu-miR-133-3p, efu-miR-133-5p, efu-miR-1343, efu-miR-135, efu-miR-136, efu-miR-1388, efu-miR-138a, efu-miR-138b, efu-miR-139, efu-miR-140, efu-miR-141, efu-miR-142, efu-miR-143, efu-miR-145, efu-miR-146, efu-miR-148, efu-miR-149, efu-miR-15, efu-miR-150, efu-miR-151, efu-miR-152, efu-miR-153, efu-miR-154, efu-miR-155, efu-miR-16, efu-miR-17, efu-miR-18, efu-miR-181a, efu-miR-181b, efu-miR-181c, efu-miR-181d, efu-miR-181e, efu-miR-181f, efu-miR-182, efu-miR-1842, efu-miR-185, efu-miR-188, efu-miR-19, efu-miR-191, efu-miR-192, efu-miR-193a, efu-miR-193b, efu-miR-194, efu-miR-196, efu-miR-199, efu-miR-20, efu-miR-200a, efu-miR-200b, efu-miR-200c, efu-miR-202, efu-miR-204, efu-miR-205, efu-miR-206, efu-miR-21, efu-miR-210, efu-miR-211, efu-miR-214, efu-miR-215, efu-miR-218a, efu-miR-218b, efu-miR-219, efu-miR-22, efu-miR-221, efu-miR-222, efu-miR-223, efu-miR-23a, efu-miR-23b, efu-miR-25, efu-miR-2683, efu-miR-26a, efu-miR-26b, efu-miR-26c, efu-miR-27a, efu-miR-27b, efu-miR-28, efu-miR-299, efu-miR-29a, efu-miR-29b, efu-miR-29c, efu-miR-30a, efu-miR-30b, efu-miR-30c, efu-miR-30d, efu-miR-30e, efu-miR-31, efu-miR-320, efu-miR-323, efu-miR-324, efu-miR-33, efu-miR-330, efu-miR-331, efu-miR-335, efu-miR-337, efu-miR-338, efu-miR-339, efu-miR-342, efu-miR-34a, efu-miR-34b, efu-miR-34c, efu-miR-361, efu-miR-365, efu-miR-370, efu-miR-375, efu-miR-376, efu-miR-378, efu-miR-379, efu-miR-381, efu-miR-382, efu-miR-383, efu-miR-3958, efu-miR-409, efu-miR-411, efu-miR-412, efu-miR-423, efu-miR-424, efu-miR-429, efu-miR-449, efu-miR-450, efu-miR-452, efu-miR-455, efu-miR-4667, efu-miR-485, efu-miR-486, efu-miR-487, efu-miR-490, efu-miR-493, efu-miR-494, efu-miR-495, efu-miR-497, efu-miR-499, efu-miR-500, efu-miR-502, efu-miR-503, efu-miR-504, efu-miR-506, efu-miR-507a, efu-miR-507b, efu-miR-507c, efu-miR-513, efu-miR-532, efu-miR-544, efu-miR-574, efu-miR-582, efu-miR-592, efu-miR-628, efu-miR-652, efu-miR-660, efu-miR-7a, efu-miR-7b, efu-miR-7c, efu-miR-874, efu-miR-8908, efu-miR-8915, efu-miR-9-3p, efu-miR-9-5p, efu-miR-9186a, efu-miR-9186b, efu-miR-9186c, efu-miR-9186d, efu-miR-9186e, efu-miR-9186f, efu-miR-9186g, efu-miR-9186h, efu-miR-9186i, efu-miR-9186j, efu-miR-9186k, efu-miR-91861, efu-miR-9186m, efu-miR-9186n, efu-miR-91860, efu-miR-9186p, efu-miR-9186q, efu-miR-9187, efu-miR-9188a, efu-miR-9188b, efu-miR-9190, efu-miR-9191, efu-miR-9192, efu-miR-9193, efu-miR-9194, efu-miR-9195, efu-miR-9196, efu-miR-9197, efu-miR-9198a, efu-miR-9198b, efu-miR-9198c, efu-miR-9198d, efu-miR-9198e, efu-miR-9199, efu-miR-9200a, efu-miR-9200b, efu-miR-9201a, efu-miR-9201b, efu-miR-9202, efu-miR-9203a, efu-miR-9203b, efu-miR-9204, efu-miR-9205, efu-miR-9206, efu-miR-9207, efu-miR-9208, efu-miR-9209a, efu-miR-9209b, efu-miR-9209c, efu-miR-9209d, efu-miR-9210, efu-miR-9211, efu-miR-9212, efu-miR-9213, efu-miR-9214, efu-miR-9215a, efu-miR-9215b, efu-miR-9216, efu-miR-9217, efu-miR-9218, efu-miR-9219, efu-miR-9220, efu-miR-9221, efu-miR-9222, efu-miR-9223a, efu-miR-9223b, efu-miR-9223c, efu-miR-9223d, efu-miR-9223e, efu-miR-9223f, efu-miR-9223g, efu-miR-9223h, efu-miR-9223i, efu-miR-9224, efu-miR-9225, efu-miR-9226, efu-miR-9227, efu-miR-9228, efu-miR-9229a, efu-miR-9229b, efu-miR-9229c, efu-miR-9229d, efu-miR-9229e, efu-miR-9229f, efu-miR-9230a, efu-miR-9230b, efu-miR-9230c, efu-miR-9231, efu-miR-9232, efu-miR-9233, efu-miR-9234a, efu-miR-9234b, efu-miR-9235a, efu-miR-9235b, efu-miR-9236a, efu-miR-9236b, efu-miR-9236c, efu-miR-9237a, efu-miR-9237b, efu-miR-9237c, efu-miR-9238, efu-miR-9239, efu-miR-9240, efu-miR-9241, efu-miR-9242, efu-miR-9243a, efu-miR-9243b, efu-miR-9243c, efu-miR-9244, efu-miR-9245, efu-miR-9246a, efu-miR-9246b, efu-miR-9246c, efu-miR-9247, efu-miR-9248, efu-miR-9249, efu-miR-9250, efu-miR-9251, efu-miR-9252, efu-miR-9253, efu-miR-9254, efu-miR-9255, efu-miR-9256a, efu-miR-9256b, efu-miR-9257, efu-miR-9258, efu-miR-9259, efu-miR-9260, efu-miR-9261, efu-miR-9262, efu-miR-9263, efu-miR-9264, efu-miR-9265, efu-miR-9266, efu-miR-9267, efu-miR-9268, efu-miR-9269, efu-miR-9270, efu-miR-9271, efu-miR-9272, efu-miR-9273, efu-miR-9274, efu-miR-9275, efu-miR-9276, efu-miR-9277, efu-miR-9278, efu-miR-9279a, efu-miR-9279b, efu-miR-9280, efu-miR-9281, efu-miR-9282, efu-miR-9283, efu-miR-9284, efu-miR-9285, efu-miR-9286, efu-miR-9287, efu-miR-9288, efu-miR-9289, efu-miR-9290, efu-miR-9291, efu-miR-9292, efu-miR-9293, efu-miR-9294, efu-miR-9295, efu-miR-9296, efu-miR-9297, efu-miR-9298, efu-miR-9299, efu-miR-92a, efu-miR-92b, efu-miR-92c, efu-miR-93, efu-miR-9300, efu-miR-9301, efu-miR-9302, efu-miR-9303, efu-miR-9304, efu-miR-9305, efu-miR-9306, efu-miR-9307, efu-miR-9308, efu-miR-9309, efu-miR-9310, efu-miR-9311, efu-miR-9312, efu-miR-9313, efu-miR-9314, efu-miR-9315, efu-miR-9316, efu-miR-9317, efu-miR-9318a, efu-miR-9318b, efu-miR-9319, efu-miR-9320, efu-miR-9321, efu-miR-9322, efu-miR-9323, efu-miR-9324, efu-miR-9325, efu-miR-9326, efu-miR-9327, efu-miR-9328, efu-miR-9329, efu-miR-9330, efu-miR-9331, efu-miR-9332, efu-miR-9333, efu-miR-9334, efu-miR-9335, efu-miR-9336, efu-miR-9337, efu-miR-9338, efu-miR-9339, efu-miR-9340, efu-miR-9341, efu-miR-9342, efu-miR-9343, efu-miR-9344, efu-miR-9345, efu-miR-9346, efu-miR-9347, efu-miR-9348, efu-miR-9349, efu-miR-9350, efu-miR-9351, efu-miR-9352, efu-miR-9353, efu-miR-9354, efu-miR-9355, efu-miR-9356, efu-miR-9357, efu-miR-9358, efu-miR-9359, efu-miR-9360, efu-miR-9361, efu-miR-9362, efu-miR-9363, efu-miR-9364, efu-miR-9365, efu-miR-9366, efu-miR-9367, efu-miR-9368, efu-miR-99a, efu-miR-99b, egr-bantam-3p, egr-bantam-5p, egr-let-7-3p, egr-let-7-5p, egr-miR-1-5p, egr-miR-10227a-3p, egr-miR-10227a-5p, egr-miR-10227b-3p, egr-miR-10227b-5p, egr-miR-10227c-3p, egr-miR-10227c-5p, egr-miR-10227d-3p, egr-miR-10227d-5p, egr-miR-10227e-3p, egr-miR-10227e-5p, egr-miR-10228-3p, egr-miR-10228-5p, egr-miR-10229a-3p, egr-miR-10229a-5p, egr-miR-10229b-3p, egr-miR-10229b-5p, egr-miR-10230-3p, egr-miR-10230-5p, egr-miR-10231-3p, egr-miR-10231-5p, egr-miR-10232-3p, egr-miR-10232-5p, egr-miR-10233-3p, egr-miR-10233-5p, egr-miR-10234-3p, egr-miR-10234-5p, egr-miR-10235-3p, egr-miR-10235-5p, egr-miR-10236-3p, egr-miR-10236-5p, egr-miR-10237-3p, egr-miR-10237-5p, egr-miR-10238-3p, egr-miR-10238-5p, egr-miR-10239-3p, egr-miR-10239-5p, egr-miR-10240-3p, egr-miR-10240-5p, egr-miR-10241-3p, egr-miR-10241-5p, egr-miR-10242-3p, egr-miR-10242-5p, egr-miR-10243-3p, egr-miR-10243-5p, egr-miR-10244-3p, egr-miR-10244-5p, egr-miR-10245-3p, egr-miR-10245-5p, egr-miR-10247-3p, egr-miR-10247-5p, egr-miR-10248-3p, egr-miR-10248-5p, egr-miR-10249-3p, egr-miR-10249-5p, egr-miR-10250-3p, egr-miR-10250-5p, egr-miR-10251-3p, egr-miR-10251-5p, egr-miR-10252-3p, egr-miR-10252-5p, egr-miR-10253-3p, egr-miR-10253-5p, egr-miR-10254-3p, egr-miR-10254-5p, egr-miR-10255-3p, egr-miR-10255-5p, egr-miR-10256-3p, egr-miR-10256-5p, egr-miR-10257-3p, egr-miR-10257-5p, egr-miR-10258-3p, egr-miR-10258-5p, egr-miR-10259-3p, egr-miR-10259-5p, egr-miR-10260-3p, egr-miR-10260-5p, egr-miR-10261-3p, egr-miR-10261-5p, egr-miR-10262-3p, egr-miR-10262-5p, egr-miR-10263-3p, egr-miR-10263-5p, egr-miR-10264-3p, egr-miR-10264-5p, egr-miR-10265-3p, egr-miR-10265-5p, egr-miR-10266-3p, egr-miR-10266-5p, egr-miR-10267-3p, egr-miR-10267-5p, egr-miR-10268-3p, egr-miR-10268-5p, egr-miR-10269-3p, egr-miR-10269-5p, egr-miR-10270-3p, egr-miR-10270-5p, egr-miR-10271-3p, egr-miR-10271-5p, egr-miR-10272-3p, egr-miR-10272-5p, egr-miR-10273-3p, egr-miR-10273-5p, egr-miR-10274-3p, egr-miR-10274-5p, egr-miR-10275-3p, egr-miR-10275-5p, egr-miR-10276-3p, egr-miR-10276-5p, egr-miR-10277-3p, egr-miR-10277-5p, egr-miR-10278-3p, egr-miR-10278-5p, egr-miR-10279-3p, egr-miR-10279-5p, egr-miR-10280-3p, egr-miR-10280-5p, egr-miR-10281-3p, egr-miR-10281-5p, egr-miR-10282-3p, egr-miR-10282-5p, egr-miR-10283-3p, egr-miR-10283-5p, egr-miR-10284-3p, egr-miR-10284-5p, egr-miR-10285-3p, egr-miR-10285-5p, egr-miR-10286-3p, egr-miR-10286-5p, egr-miR-10287-3p, egr-miR-10287-5p, egr-miR-10288-3p, egr-miR-10288-5p, egr-miR-10289-3p, egr-miR-10289-5p, egr-miR-10290-3p, egr-miR-10290-5p, egr-miR-10291-3p, egr-miR-10291-5p, egr-miR-10292-3p, egr-miR-10292-5p, egr-miR-10293-3p, egr-miR-10293-5p, egr-miR-10450-3p, egr-miR-10450-5p, egr-miR-10a-3p, egr-miR-10a-5p, egr-miR-124a-3p, egr-miR-124a-5p, egr-miR-124b-3p, egr-miR-124b-5p, egr-miR-125-3p, egr-miR-125-5p, egr-miR-133-3p, egr-miR-133-5p, egr-miR-153-3p, egr-miR-153-5p, egr-miR-184-3p, egr-miR-184-5p, egr-miR-190-3p, egr-miR-190-5p, egr-miR-1992-3p, egr-miR-1992-5p, egr-miR-2162-3p, egr-miR-2162-5p, egr-miR-219-3p, egr-miR-219-5p, egr-miR-277a-3p, egr-miR-277b-3p, egr-miR-277b-5p, egr-miR-281-3p, egr-miR-281-5p, egr-miR-2a-3p, egr-miR-2a-5p, egr-miR-2b-3p, egr-miR-2b-5p, egr-miR-2c-3p, egr-miR-2c-5p, egr-miR-2d-3p, egr-miR-2d-5p, egr-miR-307-3p, egr-miR-307-5p, egr-miR-31-3p, egr-miR-31-5p, egr-miR-3479a-3p, egr-miR-3479a-5p, egr-miR-3479b-3p, egr-miR-3479b-5p, egr-miR-36a-3p, egr-miR-36a-5p, egr-miR-36b-3p, egr-miR-36b-5p, egr-miR-4989-3p, egr-miR-4989-5p, egr-miR-4990, egr-miR-4991, egr-miR-61-3p, egr-miR-61-5p, egr-miR-7-3p, egr-miR-7-5p, egr-miR-71-3p, egr-miR-71-5p, egr-miR-745-3p, egr-miR-745-5p, egr-miR-7b-3p, egr-miR-7b-5p, egr-miR-8-3p, egr-miR-8-5p, egr-miR-87-3p, egr-miR-87-5p, egr-miR-9-3p, egr-miR-9-5p, egr-miR-96-3p, egr-miR-96-5p, egu-miR172a, egu-miR172b, egu-miR172c, egu-miR172d, egu-miR172e, egu-miR172f, emu-bantam-3p, emu-bantam-5p, emu-let-7-3p, emu-let-7-5p, emu-miR-1-3p, emu-miR-10-3p, emu-miR-10-5p, emu-miR-10293-3p, emu-miR-10293-5p, emu-miR-124a-3p, emu-miR-124a-5p, emu-miR-124b-3p, emu-miR-124b-5p, emu-miR-125-3p, emu-miR-125-5p, emu-miR-133-3p, emu-miR-133-5p, emu-miR-153-3p, emu-miR-153-5p, emu-miR-184-3p, emu-miR-184-5p, emu-miR-190-3p, emu-miR-190-5p, emu-miR-1992-3p, emu-miR-1992-5p, emu-miR-2162-3p, emu-miR-2162-5p, emu-miR-219-3p, emu-miR-219-5p, emu-miR-277a-3p, emu-miR-277b-3p, emu-miR-281-3p, emu-miR-281-5p, emu-miR-2a-3p, emu-miR-2a-5p, emu-miR-2b-3p, emu-miR-2b-5p, emu-miR-2c-3p, emu-miR-2c-5p, emu-miR-307-3p, emu-miR-307-5p, emu-miR-31-3p, emu-miR-31-5p, emu-miR-3479a-3p, emu-miR-3479a-5p, emu-miR-3479b-3p, emu-miR-3479b-5p, emu-miR-36a-3p, emu-miR-36a-5p, emu-miR-36b-3p, emu-miR-36b-5p, emu-miR-4989-3p, emu-miR-4989-5p, emu-miR-61-3p, emu-miR-61-5p, emu-miR-7-3p, emu-miR-7-5p, emu-miR-71-3p, emu-miR-71-5p, emu-miR-745-3p, emu-miR-745-5p, emu-miR-8-3p, emu-miR-8-5p, emu-miR-87-3p, emu-miR-87-5p, emu-miR-9-3p, emu-miR-9-5p, emu-miR-96-5p, esi-miR3450-3p, esi-miR3450-5p, esi-miR3451-3p, esi-miR3451-5p, esi-miR3452-3p, esi-miR3452-5p, esi-miR3453-3p, esi-miR3453-5p, esi-miR3454a-3p, esi-miR3454a-5p, esi-miR3454b-3p, esi-miR3454b-5p, esi-miR3454c-3p, esi-miR3454c-5p, esi-miR3454d-3p, esi-miR3454d-5p, esi-miR3454e-3p, esi-miR3454e-5p, esi-miR3454f-3p, esi-miR3454f-5p, esi-miR3454g-3p, esi-miR3454g-5p, esi-miR3455-3p, esi-miR3455-5p, esi-miR3455b-3p, esi-miR3455b-5p, esi-miR3456-3p, esi-miR3456-5p, esi-miR3457-3p, esi-miR3457-5p, esi-miR3458-3p, esi-miR3458-5p, esi-miR3459-3p, esi-miR3459-5p, esi-miR3460-3p, esi-miR3460-5p, esi-miR3461-3p, esi-miR3461-5p, esi-miR3462-3p, esi-miR3462-5p, esi-miR3463-3p, esi-miR3463-5p, esi-miR3464-3p, esi-miR3464-5p, esi-miR3465-3p, esi-miR3465-5p, esi-miR3466-3p, esi-miR3466-5p, esi-miR3467-3p, esi-miR3467-5p, esi-miR3468-3p, esi-miR3468-5p, esi-miR3469-3p, esi-miR3469-5p, esi-miR3469b-3p, esi-miR3469b-5p, esi-miR8618a, esi-miR8618b, esi-miR8619, esi-miR8620, esi-miR8621, esi-miR8622a, esi-miR8622b, esi-miR8622c, esi-miR8623a, esi-miR8623b, esi-miR8623c, esi-miR8623d, esi-miR8624a, esi-miR8624b, esi-miR8625, esi-miR8626, esi-miR8627, esi-miR8628, esi-miR8629, esi-miR8630, esi-miR8631, eun-miR10211a-3p, eun-miR10211a-5p, eun-miR10211b-3p, eun-miR10211b-5p, eun-miR10212-3p, eun-miR10212-5p, eun-miR10213-3p, eun-miR10213-5p, eun-miR10214a-3p, eun-miR10214a-5p, eun-miR10214b-3p, eun-miR10215-3p, eun-miR10216-3p, eun-miR10216-5p, eun-miR10217-3p, eun-miR10217-5p, eun-miR10218-3p, eun-miR10218-5p, eun-miR10219-3p, eun-miR10219-5p, eun-miR10220-3p, eun-miR10220-5p, eun-miR10221-3p, eun-miR10221-5p, eun-miR10222-3p, eun-miR10222-5p, eun-miR10223-3p, eun-miR10223-5p, eun-miR10224-3p, eun-miR159-3p, eun-miR159-5p, eun-miR160-3p, eun-miR160-5p, eun-miR162-3p, eun-miR162-5p, eun-miR166-3p, eun-miR166-5p, eun-miR167a-3p, eun-miR167a-5p, eun-miR167b-3p, eun-miR167b-5p, eun-miR167c-3p, eun-miR167c-5p, eun-miR167d-5p, eun-miR172a-3p, eun-miR172a-5p, eun-miR172b-3p, eun-miR172b-5p, eun-miR172c-3p, eun-miR172c-5p, eun-miR395-3p, eun-miR395-5p, eun-miR396a-3p, eun-miR396a-5p, eun-miR396b-3p, eun-miR396b-5p, eun-miR397a-3p, eun-miR397a-5p, eun-miR397b-3p, eun-miR397b-5p, eun-miR482a-3p, eun-miR482a-5p, eun-miR482b-3p, eun-miR482b-5p, eun-miR482c-3p, eun-miR482c-5p, eun-miR530-3p, eun-miR530-5p, eun-miR535a-3p, eun-miR535a-5p, eun-miR535b-3p, eun-miR535b-5p, eun-miR827-3p, eun-miR827-5p, far-miR1119, far-miR1122, far-miR1134, far-miR156a, far-miR156b, far-miR159, far-miR160, far-miR164a, far-miR164b, far-miR166, far-miR169, far-miR171, far-miR396, far-miR437, far-miR529, fhe-bantam, fhe-let-7, fhe-miR-1, fhe-miR-10, fhe-miR-11584, fhe-miR-11585, fhe-miR-11586, fhe-miR-11587, fhe-miR-124, fhe-miR-125a, fhe-miR-125b, fhe-miR-190, fhe-miR-2162, fhe-miR-219, fhe-miR-277, fhe-miR-2a, fhe-miR-2b, fhe-miR-2c, fhe-miR-2d, fhe-miR-2e, fhe-miR-2f, fhe-miR-307, fhe-miR-31, fhe-miR-3479, fhe-miR-36a, fhe-miR-36b, fhe-miR-46, fhe-miR-61, fhe-miR-7, fhe-miR-71a, fhe-miR-71b, fhe-miR-745a, fhe-miR-745b, fhe-miR-750, fhe-miR-8, fhe-miR-87, fhe-miR-9, fhe-miR-96, fru-let-7a, fru-let-7b, fru-let-7d, fru-let-7e, fru-let-7g, fru-let-7h, fru-let-7i, fru-let-7j, fru-miR-1, fru-miR-100, fru-miR-101a, fru-miR-101b, fru-miR-103, fru-miR-107, fru-miR-10b, fru-miR-10c, fru-miR-10d, fru-miR-122, fru-miR-124, fru-miR-125a, fru-miR-125b, fru-miR-126, fru-miR-128, fru-miR-129, fru-miR-130, fru-miR-132, fru-miR-133, fru-miR-135a, fru-miR-135b, fru-miR-137, fru-miR-138, fru-miR-140, fru-miR-142, fru-miR-144, fru-miR-148, fru-miR-152, fru-miR-153a, fru-miR-153b, fru-miR-15a, fru-miR-15b, fru-miR-16, fru-miR-17, fru-miR-18, fru-miR-181a-3p, fru-miR-181a-5p, fru-miR-181b, fru-miR-182, fru-miR-183, fru-miR-184, fru-miR-187, fru-miR-190, fru-miR-192, fru-miR-193, fru-miR-194, fru-miR-196a, fru-miR-196b, fru-miR-199, fru-miR-19a, fru-miR-19b, fru-miR-20, fru-miR-200a, fru-miR-200b, fru-miR-202, fru-miR-203, fru-miR-204a, fru-miR-205, fru-miR-21, fru-miR-210, fru-miR-212, fru-miR-214, fru-miR-216a, fru-miR-216b, fru-miR-217, fru-miR-218a, fru-miR-218b, fru-miR-219, fru-miR-221, fru-miR-222, fru-miR-223, fru-miR-22a, fru-miR-22b, fru-miR-23a, fru-miR-23b, fru-miR-24-3p, fru-miR-24-5p, fru-miR-25, fru-miR-26, fru-miR-27b, fru-miR-27c, fru-miR-27e, fru-miR-29a, fru-miR-29b, fru-miR-301, fru-miR-30b, fru-miR-30c, fru-miR-30d, fru-miR-338, fru-miR-365, fru-miR-375, fru-miR-429, fru-miR-455, fru-miR-458, fru-miR-460, fru-miR-489, fru-miR-7, fru-miR-9, fru-miR-92, fru-miR-96, fve-miR11283, fve-miR11284, fve-miR11285, fve-miR11286, fve-miR11287, fve-miR11288a, fve-miR11288b, fve-miR11288c-3p, fve-miR11288c-5p, fve-miR11288d, fve-miR11288e, fve-miR11289, fve-miR11290, fve-miR11291, fve-miR11292, fve-miR11293, fve-miR11294, fve-miR11295, fve-miR11296, fve-miR11297, fve-miR11298, fve-miR11299, fve-miR11300, fve-miR11301, fve-miR11302, fve-miR11303, fve-miR11304, fve-miR11305, fve-miR11306, fve-miR11307, fve-miR11308, fve-miR11309, fve-miR11310, fve-miR11311, fve-miR11312, fve-miR11313, fve-miR11314, fve-miR11315, fve-miR1511, fve-miR156a, fve-miR156b, fve-miR156c, fve-miR156d, fve-miR156e, fve-miR156f, fve-miR156g-3p, fve-miR156g-5p, fve-miR156h, fve-miR156i, fve-miR156j, fve-miR159a-3p, fve-miR159a-5p, fve-miR159b, fve-miR159c, fve-miR160a, fve-miR160b, fve-miR162-3p, fve-miR162-5p, fve-miR164a-3p, fve-miR164a-5p, fve-miR164b, fve-miR164c, fve-miR166a, fve-miR166b, fve-miR166c, fve-miR166d-3p, fve-miR166d-5p, fve-miR166e, fve-miR166f, fve-miR167a, fve-miR167b, fve-miR167c, fve-miR167d, fve-miR168-3p, fve-miR168-5p, fve-miR169a, fve-miR169b, fve-miR169c, fve-miR169d, fve-miR169e, fve-miR169f, fve-miR171a, fve-miR171b, fve-miR171c-3p, fve-miR171c-5p, fve-miR171d, fve-miR171e, fve-miR171f-3p, fve-miR171f-5p, fve-miR171g, fve-miR171h, fve-miR172a, fve-miR172b, fve-miR172c, fve-miR2109, fve-miR2111a, fve-miR2111b-3p, fve-miR2111b-5p, fve-miR2111c, fve-miR319, fve-miR3627a, fve-miR3627b, fve-miR390a, fve-miR390b, fve-miR393a, fve-miR393b, fve-miR394, fve-miR396a-3p, fve-miR396a-5p, fve-miR396b-3p, fve-miR396b-5p, fve-miR396c-3p, fve-miR396c-5p, fve-miR396d, fve-miR396e, fve-miR397, fve-miR399a, fve-miR399b, fve-miR408, fve-miR477a, fve-miR477b, fve-miR482a, fve-miR482b, fve-miR482c, fve-miR482d, fve-miR5225, fve-miR530, fve-miR535a, fve-miR535b, fve-miR827, fve-miR845, gar-miR2947, gga-let-7a-2-3p, gga-let-7a-3p, gga-let-7a-5p, gga-let-7b, gga-let-7c-3p, gga-let-7c-5p, gga-let-7d, gga-let-7f-3p, gga-let-7f-5p, gga-let-7g-3p, gga-let-7g-5p, gga-let-7i, gga-let-7j-3p, gga-let-7j-5p, gga-let-7k-3p, gga-let-7k-5p, gga-let-71-3p, gga-let-71-5p, gga-miR-100-3p, gga-miR-100-5p, gga-miR-101-1-5p, gga-miR-101-2-5p, gga-miR-101-3p, gga-miR-103-1-5p, gga-miR-103-2-5p, gga-miR-103-3p, gga-miR-10585-3p, gga-miR-10585-5p, gga-miR-106-3p, gga-miR-106-5p, gga-miR-107-3p, gga-miR-107-5p, gga-miR-10a-3p, gga-miR-10a-5p, gga-miR-10b-3p, gga-miR-10b-5p, gga-miR-10c-3p, gga-miR-10c-5p, gga-miR-122-3p, gga-miR-122-5p, gga-miR-12207-3p, gga-miR-12207-5p, gga-miR-12208-3p, gga-miR-12208-5p, gga-miR-12209-3p, gga-miR-12209-5p, gga-miR-12210-3p, gga-miR-12210-5p, gga-miR-12211-3p, gga-miR-12211-5p, gga-miR-12212-5p, gga-miR-12213-3p, gga-miR-12213-5p, gga-miR-12214-3p, gga-miR-12214-5p, gga-miR-12215-5p, gga-miR-12216-3p, gga-miR-12216-5p, gga-miR-12217-3p, gga-miR-12218-3p, gga-miR-12218-5p, gga-miR-12219-3p, gga-miR-12219-5p, gga-miR-12220-3p, gga-miR-12220-5p, gga-miR-12221-2-3p, gga-miR-12221-3p, gga-miR-12221-5p, gga-miR-12222-3p, gga-miR-12222-5p, gga-miR-12223-3p, gga-miR-12223-5p, gga-miR-12224-3p, gga-miR-12224-5p, gga-miR-12225-3p, gga-miR-12225-5p, gga-miR-12226-3p, gga-miR-12226-5p, gga-miR-12227-3p, gga-miR-12227-5p, gga-miR-12228-3p, gga-miR-12228-5p, gga-miR-12229-3p, gga-miR-12229-5p, gga-miR-12230-5p, gga-miR-12231-3p, gga-miR-12231-5p, gga-miR-12232-3p, gga-miR-12232-5p, gga-miR-12233-3p, gga-miR-12233-5p, gga-miR-12234-3p, gga-miR-12234-5p, gga-miR-12235-3p, gga-miR-12235-5p, gga-miR-12236-3p, gga-miR-12236-5p, gga-miR-12237-3p, gga-miR-12237-5p, gga-miR-12238-3p, gga-miR-12239-3p, gga-miR-12239-5p, gga-miR-12240-3p, gga-miR-12240-5p, gga-miR-12241-3p, gga-miR-12242-3p, gga-miR-12242-5p, gga-miR-12243-3p, gga-miR-12243-5p, gga-miR-12244-5p, gga-miR-12245-3p, gga-miR-12245-5p, gga-miR-12246-3p, gga-miR-12246-5p, gga-miR-12247-3p, gga-miR-12247-5p, gga-miR-12248-3p, gga-miR-12248-5p, gga-miR-12249-3p, gga-miR-12249-5p, gga-miR-12250-3p, gga-miR-12250-5p, gga-miR-12251-3p, gga-miR-12251-5p, gga-miR-12252-3p, gga-miR-12252-5p, gga-miR-12253-3p, gga-miR-12253-5p, gga-miR-12254-3p, gga-miR-12254-5p, gga-miR-12255-3p, gga-miR-12255-5p, gga-miR-12256-3p, gga-miR-12256-5p, gga-miR-12257-3p, gga-miR-12257-5p, gga-miR-12258-3p, gga-miR-12258-5p, gga-miR-12259-3p, gga-miR-12259-5p, gga-miR-12260-3p, gga-miR-12260-5p, gga-miR-12261-3p, gga-miR-12261-5p, gga-miR-12262-5p, gga-miR-12263-3p, gga-miR-12263-5p, gga-miR-12264-3p, gga-miR-12264-5p, gga-miR-12265-3p, gga-miR-12265-5p, gga-miR-12266-3p, gga-miR-12266-5p, gga-miR-12267-3p, gga-miR-12267-5p, gga-miR-12268-5p, gga-miR-12269-3p, gga-miR-12269-5p, gga-miR-12270-3p, gga-miR-12270-5p, gga-miR-12271-3p, gga-miR-12271-5p, gga-miR-12272-3p, gga-miR-12272-5p, gga-miR-12273-3p, gga-miR-12273-5p, gga-miR-12274-3p, gga-miR-12274-5p, gga-miR-12275-3p, gga-miR-12275-5p, gga-miR-12276-3p, gga-miR-12276-5p, gga-miR-12277-3p, gga-miR-12277-5p, gga-miR-12278-3p, gga-miR-12278-5p, gga-miR-12279-3p, gga-miR-12279-5p, gga-miR-12280-3p, gga-miR-12280-5p, gga-miR-12281-3p, gga-miR-12282-3p, gga-miR-12282-5p, gga-miR-12283-3p, gga-miR-12283-5p, gga-miR-12284-3p, gga-miR-12284-5p, gga-miR-12285-3p, gga-miR-12285-5p, gga-miR-12286-3p, gga-miR-12286-5p, gga-miR-12287-5p, gga-miR-12288-3p, gga-miR-12288-5p, gga-miR-12289-3p, gga-miR-12289-5p, gga-miR-12290-3p, gga-miR-12290-5p, gga-miR-12291-3p, gga-miR-12292-3p, gga-miR-12292-5p, gga-miR-12293-3p, gga-miR-12293-5p, gga-miR-12294-3p, gga-miR-12294-5p, gga-miR-12295-3p, gga-miR-12295-5p, gga-miR-122b, gga-miR-122b-3p, gga-miR-122b-5p, gga-miR-124a-3p, gga-miR-124a-5p, gga-miR-124b, gga-miR-124c-3p, gga-miR-124c-5p, gga-miR-125b-3p, gga-miR-125b-5p, gga-miR-126-3p, gga-miR-126-5p, gga-miR-128-1-5p, gga-miR-128-2-5p, gga-miR-128-3p, gga-miR-129-1-3p, gga-miR-129-3p, gga-miR-129-5p, gga-miR-1306-3p, gga-miR-1306-5p, gga-miR-130a-3p, gga-miR-130a-5p, gga-miR-130b-3p, gga-miR-130b-5p, gga-miR-130c-3p, gga-miR-130c-5p, gga-miR-1329-3p, gga-miR-1329-5p, gga-miR-132a-3p, gga-miR-132a-5p, gga-miR-132b-3p, gga-miR-132b-5p, gga-miR-132c-3p, gga-miR-132c-5p, gga-miR-133a-3p, gga-miR-133a-5p, gga-miR-133b, gga-miR-133c-3p, gga-miR-133c-5p, gga-miR-1354, gga-miR-135a-1-3p, gga-miR-135a-2-3p, gga-miR-135a-3-3p, gga-miR-135a-5p, gga-miR-135b, gga-miR-137-3p, gga-miR-137-5p, gga-miR-138-1-3p, gga-miR-138-2-3p, gga-miR-138-5p, gga-miR-1388a-3p, gga-miR-1388a-5p, gga-miR-1388b-3p, gga-miR-1388b-5p, gga-miR-1397-3p, gga-miR-1397-5p, gga-miR-140-3p, gga-miR-140-5p, gga-miR-1416-3p, gga-miR-1416-5p, gga-miR-142-3p, gga-miR-142-5p, gga-miR-143-3p, gga-miR-143-5p, gga-miR-1434, gga-miR-144-3p, gga-miR-144-5p, gga-miR-145-3p, gga-miR-145-5p, gga-miR-1451-3p, gga-miR-1451-5p, gga-miR-1452, gga-miR-1453, gga-miR-1454, gga-miR-1456-3p, gga-miR-1456-5p, gga-miR-1457, gga-miR-1458, gga-miR-1459, gga-miR-1460, gga-miR-1462-3p, gga-miR-1462-5p, gga-miR-1463, gga-miR-1464, gga-miR-1465, gga-miR-1466, gga-miR-1467-3p, gga-miR-1467-5p, gga-miR-146a-3p, gga-miR-146a-5p, gga-miR-146b-3p, gga-miR-146b-5p, gga-miR-146c-3p, gga-miR-146c-5p, gga-miR-147, gga-miR-148a-3p, gga-miR-148a-5p, gga-miR-148b-3p, gga-miR-148b-5p, gga-miR-153-3p, gga-miR-153-5p, gga-miR-155, gga-miR-1550-3p, gga-miR-1550-5p, gga-miR-1551-3p, gga-miR-1551-5p, gga-miR-1552-3p, gga-miR-1552-5p, gga-miR-1553-3p, gga-miR-1553-5p, gga-miR-1554, gga-miR-1555-3p, gga-miR-1555-5p, gga-miR-1556, gga-miR-1557, gga-miR-1558, gga-miR-1559-3p, gga-miR-1559-5p, gga-miR-1560-3p, gga-miR-1560-5p, gga-miR-1561, gga-miR-1562-3p, gga-miR-1562-5p, gga-miR-1563, gga-miR-1564-3p, gga-miR-1564-5p, gga-miR-1565, gga-miR-1567, gga-miR-1568, gga-miR-1569, gga-miR-1570, gga-miR-1571, gga-miR-1572, gga-miR-1573, gga-miR-1574-3p, gga-miR-1574-5p, gga-miR-1575, gga-miR-1576, gga-miR-1577, gga-miR-1578, gga-miR-1579, gga-miR-1580, gga-miR-1581, gga-miR-1582, gga-miR-1583, gga-miR-1584, gga-miR-1585, gga-miR-1586, gga-miR-1587, gga-miR-1588, gga-miR-1589, gga-miR-1590, gga-miR-1591-3p, gga-miR-1591-5p, gga-miR-1592, gga-miR-1593, gga-miR-1594, gga-miR-1595-3p, gga-miR-1595-5p, gga-miR-1596-3p, gga-miR-1596-5p, gga-miR-1597-3p, gga-miR-1597-5p, gga-miR-1598, gga-miR-1599, gga-miR-15a, gga-miR-15b-3p, gga-miR-15b-5p, gga-miR-15c-3p, gga-miR-15c-5p, gga-miR-16-1-3p, gga-miR-16-2-3p, gga-miR-16-5p, gga-miR-1600, gga-miR-1601, gga-miR-1602, gga-miR-1603, gga-miR-1604, gga-miR-1605, gga-miR-1606, gga-miR-1607, gga-miR-1608, gga-miR-1609, gga-miR-1610, gga-miR-1611, gga-miR-1612, gga-miR-1613, gga-miR-1614-3p, gga-miR-1614-5p, gga-miR-1615, gga-miR-1616, gga-miR-1617, gga-miR-1618-3p, gga-miR-1618-5p, gga-miR-1619, gga-miR-1620, gga-miR-1621-3p, gga-miR-1621-5p, gga-miR-1622, gga-miR-1623, gga-miR-1624, gga-miR-1625-3p, gga-miR-1625-5p, gga-miR-1626-3p, gga-miR-1626-5p, gga-miR-1627-3p, gga-miR-1627-5p, gga-miR-1628, gga-miR-1629, gga-miR-1630, gga-miR-1631, gga-miR-1632-3p, gga-miR-1632-5p, gga-miR-1633, gga-miR-1634, gga-miR-1635, gga-miR-1636, gga-miR-1637, gga-miR-1638, gga-miR-1639, gga-miR-1640, gga-miR-1641, gga-miR-1642, gga-miR-1643-3p, gga-miR-1643-5p, gga-miR-1644, gga-miR-1645, gga-miR-1646, gga-miR-1647, gga-miR-1648-3p, gga-miR-1648-5p, gga-miR-1649-3p, gga-miR-1649-5p, gga-miR-1650, gga-miR-1651-3p, gga-miR-1651-5p, gga-miR-1652, gga-miR-1653, gga-miR-1654, gga-miR-1655-3p, gga-miR-1655-5p, gga-miR-1656, gga-miR-1657, gga-miR-1658-3p, gga-miR-1658-5p, gga-miR-1659, gga-miR-1660, gga-miR-1661, gga-miR-1662, gga-miR-1663-3p, gga-miR-1663-5p, gga-miR-1664-3p, gga-miR-1664-5p, gga-miR-1665, gga-miR-1666, gga-miR-1667-3p, gga-miR-1667-5p, gga-miR-1668-3p, gga-miR-1668-5p, gga-miR-1669, gga-miR-1670, gga-miR-1671, gga-miR-1672, gga-miR-1673, gga-miR-1674, gga-miR-1675, gga-miR-1676-3p, gga-miR-1676-5p, gga-miR-1677-3p, gga-miR-1677-5p, gga-miR-1678, gga-miR-1679, gga-miR-1680-3p, gga-miR-1680-5p, gga-miR-1681, gga-miR-1682, gga-miR-1683, gga-miR-1684a-3p, gga-miR-1684a-5p, gga-miR-1684b-3p, gga-miR-1685-3p, gga-miR-1685-5p, gga-miR-1686, gga-miR-1687-3p, gga-miR-1687-5p, gga-miR-1688, gga-miR-1689-3p, gga-miR-1689-5p, gga-miR-1690-5p, gga-miR-1691, gga-miR-1692, gga-miR-1693, gga-miR-1694, gga-miR-1695, gga-miR-1696, gga-miR-1697, gga-miR-1698, gga-miR-1699, gga-miR-16c-3p, gga-miR-16c-5p, gga-miR-17-3p, gga-miR-17-5p, gga-miR-1700, gga-miR-1701, gga-miR-1702, gga-miR-1703-3p, gga-miR-1703-5p, gga-miR-1704, gga-miR-1705, gga-miR-1706, gga-miR-1707, gga-miR-1708, gga-miR-1709, gga-miR-1710, gga-miR-1711, gga-miR-1712-3p, gga-miR-1712-5p, gga-miR-1713, gga-miR-1714, gga-miR-1715-3p, gga-miR-1715-5p, gga-miR-1716, gga-miR-1717, gga-miR-1718, gga-miR-1719, gga-miR-1720-3p, gga-miR-1720-5p, gga-miR-1721, gga-miR-1722-3p, gga-miR-1722-5p, gga-miR-1723, gga-miR-1724, gga-miR-1725, gga-miR-1726, gga-miR-1727, gga-miR-1728-3p, gga-miR-1728-5p, gga-miR-1729-3p, gga-miR-1729-5p, gga-miR-1730-3p, gga-miR-1730-5p, gga-miR-1731-3p, gga-miR-1731-5p, gga-miR-1732, gga-miR-1733, gga-miR-1734, gga-miR-1735, gga-miR-1736-3p, gga-miR-1736-5p, gga-miR-1737, gga-miR-1738, gga-miR-1739, gga-miR-1740-3p, gga-miR-1740-5p, gga-miR-1741, gga-miR-1742, gga-miR-1743, gga-miR-1744-3p, gga-miR-1744-5p, gga-miR-1745, gga-miR-1746, gga-miR-1747-3p, gga-miR-1747-5p, gga-miR-1748, gga-miR-1749-3p, gga-miR-1749-5p, gga-miR-1750, gga-miR-1751-5p, gga-miR-1752, gga-miR-1753, gga-miR-1754-3p, gga-miR-1754-5p, gga-miR-1755, gga-miR-1756a, gga-miR-1756b, gga-miR-1757, gga-miR-1758, gga-miR-1759-3p, gga-miR-1759-5p, gga-miR-1760, gga-miR-1761, gga-miR-1762, gga-miR-1763, gga-miR-1763b-3p, gga-miR-1763b-5p, gga-miR-1764-3p, gga-miR-1764-5p, gga-miR-1765, gga-miR-1766, gga-miR-1767, gga-miR-1768, gga-miR-1769-3p, gga-miR-1769-5p, gga-miR-1770, gga-miR-1771, gga-miR-1772-3p, gga-miR-1772-5p, gga-miR-1773-3p, gga-miR-1773-5p, gga-miR-1774, gga-miR-1775-3p, gga-miR-1775-5p, gga-miR-1776, gga-miR-1777, gga-miR-1778, gga-miR-1779, gga-miR-1780, gga-miR-1781-3p, gga-miR-1781-5p, gga-miR-1782, gga-miR-1783, gga-miR-1784-5p, gga-miR-1784b-3p, gga-miR-1784b-5p, gga-miR-1785, gga-miR-1786, gga-miR-1787, gga-miR-1788-3p, gga-miR-1788-5p, gga-miR-1789, gga-miR-1790, gga-miR-1791-3p, gga-miR-1791-5p, gga-miR-1792, gga-miR-1793, gga-miR-1794, gga-miR-1795, gga-miR-1796, gga-miR-1797, gga-miR-1798-3p, gga-miR-1798-5p, gga-miR-1799, gga-miR-1800, gga-miR-1801, gga-miR-1802, gga-miR-1803, gga-miR-1804, gga-miR-1805-3p, gga-miR-1805-5p, gga-miR-1806, gga-miR-1807, gga-miR-1808, gga-miR-1809, gga-miR-1811, gga-miR-1812-3p, gga-miR-1812-5p, gga-miR-1813, gga-miR-1814, gga-miR-1815, gga-miR-1816, gga-miR-181a-3p, gga-miR-181a-5p, gga-miR-181b-1-3p, gga-miR-181b-2-3p, gga-miR-181b-5p, gga-miR-182-3p, gga-miR-182-5p, gga-miR-183, gga-miR-184-3p, gga-miR-184-5p, gga-miR-1845, gga-miR-184b-3p, gga-miR-184b-5p, gga-miR-187-3p, gga-miR-187-5p, gga-miR-18a-3p, gga-miR-18a-5p, gga-miR-18b-3p, gga-miR-18b-5p, gga-miR-190a-3p, gga-miR-190a-5p, gga-miR-190b-3p, gga-miR-190b-5p, gga-miR-191-3p, gga-miR-191-5p, gga-miR-193a-3p, gga-miR-193a-5p, gga-miR-193b-3p, gga-miR-193b-5p, gga-miR-194, gga-miR-196-1-3p, gga-miR-196-2-3p, gga-miR-196-5p, gga-miR-199-3p, gga-miR-199-5p, gga-miR-199b, gga-miR-19a-3p, gga-miR-19a-5p, gga-miR-19b-3p, gga-miR-19b-5p, gga-miR-1a-1-5p, gga-miR-1a-2-5p, gga-miR-1a-3p, gga-miR-1b-3p, gga-miR-1b-5p, gga-miR-1c, gga-miR-200a-3p, gga-miR-200a-5p, gga-miR-200b-3p, gga-miR-200b-5p, gga-miR-202-3p, gga-miR-202-5p, gga-miR-203a, gga-miR-203b-3p, gga-miR-204, gga-miR-205a, gga-miR-205b, gga-miR-205c-3p, gga-miR-205c-5p, gga-miR-206, gga-miR-20a-3p, gga-miR-20a-5p, gga-miR-20b-3p, gga-miR-20b-5p, gga-miR-21-3p, gga-miR-21-5p, gga-miR-210a-3p, gga-miR-210a-5p, gga-miR-210b-3p, gga-miR-210b-5p, gga-miR-211, gga-miR-212-3p, gga-miR-212-5p, gga-miR-2126, gga-miR-2127, gga-miR-2128, gga-miR-2129, gga-miR-2130, gga-miR-2131-3p, gga-miR-2131-5p, gga-miR-214, gga-miR-214b-3p, gga-miR-214b-5p, gga-miR-215-3p, gga-miR-215-5p, gga-miR-216a, gga-miR-216b, gga-miR-216c, gga-miR-217-3p, gga-miR-217-5p, gga-miR-218-3p, gga-miR-218-5p, gga-miR-2184a-3p, gga-miR-2184a-5p, gga-miR-2184b-3p, gga-miR-2184b-5p, gga-miR-2188-3p, gga-miR-2188-5p, gga-miR-219a, gga-miR-219b, gga-miR-22-3p, gga-miR-22-5p, gga-miR-221-3p, gga-miR-221-5p, gga-miR-222a, gga-miR-222b-3p, gga-miR-222b-5p, gga-miR-223, gga-miR-23b-3p, gga-miR-23b-5p, gga-miR-24-3p, gga-miR-24-5p, gga-miR-26a-2-5p, gga-miR-26a-3p, gga-miR-26a-5p, gga-miR-27b-3p, gga-miR-27b-5p, gga-miR-2954, gga-miR-2984-3p, gga-miR-2984-5p, gga-miR-2985-2-3p, gga-miR-2985-3p, gga-miR-2985-5p, gga-miR-29a-3p, gga-miR-29a-5p, gga-miR-29b-1-5p, gga-miR-29b-2-5p, gga-miR-29b-3p, gga-miR-29c-3p, gga-miR-29c-5p, gga-miR-301a-3p, gga-miR-301a-5p, gga-miR-301b-3p, gga-miR-301b-5p, gga-miR-302a, gga-miR-302b-3p, gga-miR-302b-5p, gga-miR-302c-3p, gga-miR-302c-5p, gga-miR-302d, gga-miR-3064-3p, gga-miR-3064-5p, gga-miR-30a-3p, gga-miR-30a-5p, gga-miR-30b-3p, gga-miR-30b-5p, gga-miR-30c-1-3p, gga-miR-30c-2-3p, gga-miR-30c-5p, gga-miR-30d, gga-miR-30e-3p, gga-miR-30e-5p, gga-miR-31-3p, gga-miR-31-5p, gga-miR-32-3p, gga-miR-32-5p, gga-miR-33-2-5p, gga-miR-33-3p, gga-miR-33-5p, gga-miR-338-3p, gga-miR-338-5p, gga-miR-34a-3p, gga-miR-34a-5p, gga-miR-34b-3p, gga-miR-34b-5p, gga-miR-34c-3p, gga-miR-34c-5p, gga-miR-3523, gga-miR-3524a, gga-miR-3524b-3p, gga-miR-3525, gga-miR-3526, gga-miR-3528, gga-miR-3529, gga-miR-3530-3p, gga-miR-3530-5p, gga-miR-3531-3p, gga-miR-3531-5p, gga-miR-3532-3p, gga-miR-3532-5p, gga-miR-3533, gga-miR-3534, gga-miR-3535, gga-miR-3536, gga-miR-3537, gga-miR-3538, gga-miR-3539, gga-miR-3540, gga-miR-3594-3p, gga-miR-3594-5p, gga-miR-363-3p, gga-miR-363-5p, gga-miR-365-1-5p, gga-miR-365-2-5p, gga-miR-365-3p, gga-miR-365b-5p, gga-miR-367, gga-miR-375, gga-miR-383-3p, gga-miR-383-5p, gga-miR-425-3p, gga-miR-425-5p, gga-miR-429-3p, gga-miR-429-5p, gga-miR-449a, gga-miR-449b-3p, gga-miR-449b-5p, gga-miR-449c-3p, gga-miR-449c-5p, gga-miR-449d-3p, gga-miR-449d-5p, gga-miR-451, gga-miR-454-3p, gga-miR-454-5p, gga-miR-455-3p, gga-miR-455-5p, gga-miR-456-3p, gga-miR-456-5p, gga-miR-458a-3p, gga-miR-458a-5p, gga-miR-458b-3p, gga-miR-458b-5p, gga-miR-460a-3p, gga-miR-460a-5p, gga-miR-460b-3p, gga-miR-460b-5p, gga-miR-466, gga-miR-4732-5p, gga-miR-489-3p, gga-miR-489-5p, gga-miR-490-3p, gga-miR-490-5p, gga-miR-499-3p, gga-miR-499-5p, gga-miR-551-3p, gga-miR-551-5p, gga-miR-6516-3p, gga-miR-6516-5p, gga-miR-6542-3p, gga-miR-6542-5p, gga-miR-6543-3p, gga-miR-6543-5p, gga-miR-6544-3p, gga-miR-6544-5p, gga-miR-6545-3p, gga-miR-6545-5p, gga-miR-6546-3p, gga-miR-6546-5p, gga-miR-6547-3p, gga-miR-6547-5p, gga-miR-6548-3p, gga-miR-6548-5p, gga-miR-6549-3p, gga-miR-6549-5p, gga-miR-6550-3p, gga-miR-6550-5p, gga-miR-6551-3p, gga-miR-6551-5p, gga-miR-6552-3p, gga-miR-6552-5p, gga-miR-6553-3p, gga-miR-6553-5p, gga-miR-6554-3p, gga-miR-6554-5p, gga-miR-6555-3p, gga-miR-6555-5p, gga-miR-6556-3p, gga-miR-6556-5p, gga-miR-6557-3p, gga-miR-6557-5p, gga-miR-6558-3p, gga-miR-6558-5p, gga-miR-6559-3p, gga-miR-6559-5p, gga-miR-6560-3p, gga-miR-6560-5p, gga-miR-6561-3p, gga-miR-6561-5p, gga-miR-6562-3p, gga-miR-6562-5p, gga-miR-6563-3p, gga-miR-6563-5p, gga-miR-6564-3p, gga-miR-6564-5p, gga-miR-6565-3p, gga-miR-6565-5p, gga-miR-6566-3p, gga-miR-6566-5p, gga-miR-6567-3p, gga-miR-6567-5p, gga-miR-6568-3p, gga-miR-6568-5p, gga-miR-6569-3p, gga-miR-6569-5p, gga-miR-6570-3p, gga-miR-6570-5p, gga-miR-6571-3p, gga-miR-6571-5p, gga-miR-6572-3p, gga-miR-6572-5p, gga-miR-6573-3p, gga-miR-6573-5p, gga-miR-6574-3p, gga-miR-6574-5p, gga-miR-6575-3p, gga-miR-6575-5p, gga-miR-6576-3p, gga-miR-6576-5p, gga-miR-6577-3p, gga-miR-6577-5p, gga-miR-6578-3p, gga-miR-6578-5p, gga-miR-6579-3p, gga-miR-6579-5p, gga-miR-6580-3p, gga-miR-6580-5p, gga-miR-6581-3p, gga-miR-6581-5p, gga-miR-6582-3p, gga-miR-6582-5p, gga-miR-6583-5p, gga-miR-6584-5p, gga-miR-6585-5p, gga-miR-6586-5p, gga-miR-6587-3p, gga-miR-6587-5p, gga-miR-6588-3p, gga-miR-6589-5p, gga-miR-6590-3p, gga-miR-6590-5p, gga-miR-6591-3p, gga-miR-6592-3p, gga-miR-6593-3p, gga-miR-6593-5p, gga-miR-6594-5p, gga-miR-6595-5p, gga-miR-6596-3p, gga-miR-6596-5p, gga-miR-6597-3p, gga-miR-6598-5p, gga-miR-6599-3p, gga-miR-6599-5p, gga-miR-6600-3p, gga-miR-6601-3p, gga-miR-6602-5p, gga-miR-6603-3p, gga-miR-6604-5p, gga-miR-6605-5p, gga-miR-6606-5p, gga-miR-6607-5p, gga-miR-6608-3p, gga-miR-6608-5p, gga-miR-6609-3p, gga-miR-6610-3p, gga-miR-6611-5p, gga-miR-6612-5p, gga-miR-6613-3p, gga-miR-6614-3p, gga-miR-6615-3p, gga-miR-6615-5p, gga-miR-6616-5p, gga-miR-6617-3p, gga-miR-6618-5p, gga-miR-6619-5p, gga-miR-6620-5p, gga-miR-6621-5p, gga-miR-6622-3p, gga-miR-6623-3p, gga-miR-6624-3p, gga-miR-6625-5p, gga-miR-6626-5p, gga-miR-6627-3p, gga-miR-6628-3p, gga-miR-6629-5p, gga-miR-6630-3p, gga-miR-6631-5p, gga-miR-6632-5p, gga-miR-6633-5p, gga-miR-6634-5p, gga-miR-6635-5p, gga-miR-6637-3p, gga-miR-6639-5p, gga-miR-6640-5p, gga-miR-6641-3p, gga-miR-6641-5p, gga-miR-6642-3p, gga-miR-6642-5p, gga-miR-6643-5p, gga-miR-6644-3p, gga-miR-6645-5p, gga-miR-6646-3p, gga-miR-6647-5p, gga-miR-6648-3p, gga-miR-6648-5p, gga-miR-6649-3p, gga-miR-6649-5p, gga-miR-6650-5p, gga-miR-6651-5p, gga-miR-6652-5p, gga-miR-6653-3p, gga-miR-6654-3p, gga-miR-6655-5p, gga-miR-6656-5p, gga-miR-6657-3p, gga-miR-6658-3p, gga-miR-6659-3p, gga-miR-6660-3p, gga-miR-6661-5p, gga-miR-6662-3p, gga-miR-6663-5p, gga-miR-6664-3p, gga-miR-6665-5p, gga-miR-6666-3p, gga-miR-6667-5p, gga-miR-6668-3p, gga-miR-6669-3p, gga-miR-6669-5p, gga-miR-6670-5p, gga-miR-6671-5p, gga-miR-6672-3p, gga-miR-6673-3p, gga-miR-6674-3p, gga-miR-6675-3p, gga-miR-6675-5p, gga-miR-6676-3p, gga-miR-6677-3p, gga-miR-6677-5p, gga-miR-6678-3p, gga-miR-6679-5p, gga-miR-6680-3p, gga-miR-6681-5p, gga-miR-6682-3p, gga-miR-6683-3p, gga-miR-6684-5p, gga-miR-6685-3p, gga-miR-6685-5p, gga-miR-6686-3p, gga-miR-6687-3p, gga-miR-6688-3p, gga-miR-6689-3p, gga-miR-6690-5p, gga-miR-6691-5p, gga-miR-6692-5p, gga-miR-6693-3p, gga-miR-6694-3p, gga-miR-6695-5p, gga-miR-6696-3p, gga-miR-6696-5p, gga-miR-6697-5p, gga-miR-6698-3p, gga-miR-6699-5p, gga-miR-6700-3p, gga-miR-6700-5p, gga-miR-6701-3p, gga-miR-6702-5p, gga-miR-6703-3p, gga-miR-6704-5p, gga-miR-6705-5p, gga-miR-6706-5p, gga-miR-6707-5p, gga-miR-6708-5p, gga-miR-6709-5p, gga-miR-6710-3p, gga-miR-6711-5p, gga-miR-6712-5p, gga-miR-6713-3p, gga-miR-6714-3p, gga-miR-7, gga-miR-726-3p, gga-miR-726-5p, gga-miR-7437-3p, gga-miR-7437-5p, gga-miR-7438-3p, gga-miR-7438-5p, gga-miR-7439-3p, gga-miR-7439-5p, gga-miR-7440-3p, gga-miR-7440-5p, gga-miR-7441-3p, gga-miR-7441-5p, gga-miR-7442-3p, gga-miR-7442-5p, gga-miR-7443-3p, gga-miR-7443-5p, gga-miR-7444-3p, gga-miR-7444-5p, gga-miR-7445-3p, gga-miR-7445-5p, gga-miR-7446-3p, gga-miR-7446-5p, gga-miR-7447-3p, gga-miR-7447-5p, gga-miR-7448-3p, gga-miR-7448-5p, gga-miR-7449-3p, gga-miR-7449-5p, gga-miR-7450-3p, gga-miR-7450-5p, gga-miR-7451-3p, gga-miR-7451-5p, gga-miR-7452-3p, gga-miR-7452-5p, gga-miR-7453-3p, gga-miR-7453-5p, gga-miR-7454-3p, gga-miR-7454-5p, gga-miR-7455-3p, gga-miR-7455-5p, gga-miR-7456-3p, gga-miR-7456-5p, gga-miR-7457-3p, gga-miR-7457-5p, gga-miR-7458-3p, gga-miR-7458-5p, gga-miR-7459-3p, gga-miR-7459-5p, gga-miR-7460-3p, gga-miR-7460-5p, gga-miR-7461-3p, gga-miR-7461-5p, gga-miR-7462-3p, gga-miR-7462-5p, gga-miR-7463-3p, gga-miR-7463-5p, gga-miR-7464-3p, gga-miR-7464-5p, gga-miR-7465-3p, gga-miR-7465-5p, gga-miR-7466-3p, gga-miR-7466-5p, gga-miR-7467-3p, gga-miR-7467-5p, gga-miR-7468-3p, gga-miR-7468-5p, gga-miR-7469-3p, gga-miR-7469-5p, gga-miR-7470-3p, gga-miR-7470-5p, gga-miR-7471-3p, gga-miR-7471-5p, gga-miR-7472-3p, gga-miR-7472-5p, gga-miR-7473-3p, gga-miR-7473-5p, gga-miR-7474-3p, gga-miR-7474-5p, gga-miR-7475-3p, gga-miR-7475-5p, gga-miR-7476-3p, gga-miR-7476-5p, gga-miR-7477-3p, gga-miR-7477-5p, gga-miR-7478-3p, gga-miR-7478-5p, gga-miR-7479-3p, gga-miR-7479-5p, gga-miR-7480-3p, gga-miR-7480-5p, gga-miR-7481-3p, gga-miR-7481-5p, gga-miR-7482-3p, gga-miR-7482-5p, gga-miR-7483-3p, gga-miR-7483-5p, gga-miR-757, gga-miR-762, gga-miR-7b, gga-miR-9-3p, gga-miR-9-4-3p, gga-miR-9-5p, gga-miR-92-3p, gga-miR-92-5p, gga-miR-96-3p, gga-miR-96-5p, gga-miR-99a-3p, gga-miR-99a-5p, gga-miR-9b-3p, gga-miR-9b-5p, gggpv1-miR-P1-3p, gggpv1-miR-P1-5p, ggo-let-7a, ggo-let-7b, ggo-let-7c, ggo-let-7e, ggo-let-7f, ggo-let-7g, ggo-let-7i, ggo-miR-1, ggo-miR-100, ggo-miR-101, ggo-miR-103, ggo-miR-105, ggo-miR-106a, ggo-miR-106b, ggo-miR-107, ggo-miR-10a, ggo-miR-10b, ggo-miR-1179, ggo-miR-1180, ggo-miR-1185, ggo-miR-1193, ggo-miR-1197, ggo-miR-122, ggo-miR-1228, ggo-miR-1249, ggo-miR-124a, ggo-miR-1250, ggo-miR-1252, ggo-miR-1255a, ggo-miR-125a, ggo-miR-125b, ggo-miR-1260b, ggo-miR-1264, ggo-miR-1266, ggo-miR-127, ggo-miR-1271, ggo-miR-1277, ggo-miR-1278, ggo-miR-128, ggo-miR-1283, ggo-miR-1286, ggo-miR-1287, ggo-miR-1289, ggo-miR-129, ggo-miR-1291, ggo-miR-1296, ggo-miR-1298, ggo-miR-1301, ggo-miR-1304, ggo-miR-1306, ggo-miR-1307, ggo-miR-130a, ggo-miR-130b, ggo-miR-133a, ggo-miR-133b, ggo-miR-134, ggo-miR-1343, ggo-miR-135a, ggo-miR-135b, ggo-miR-136, ggo-miR-138, ggo-miR-139, ggo-miR-140, ggo-miR-141, ggo-miR-142, ggo-miR-143, ggo-miR-144, ggo-miR-145, ggo-miR-146a, ggo-miR-146b, ggo-miR-148a, ggo-miR-148b, ggo-miR-149, ggo-miR-150, ggo-miR-151a, ggo-miR-152, ggo-miR-153, ggo-miR-154, ggo-miR-155, ggo-miR-15a, ggo-miR-15b, ggo-miR-16, ggo-miR-17-3p, ggo-miR-17-5p, ggo-miR-181a-3p, ggo-miR-181a-5p, ggo-miR-181b, ggo-miR-181c, ggo-miR-181d, ggo-miR-182, ggo-miR-183, ggo-miR-185, ggo-miR-186, ggo-miR-187, ggo-miR-188, ggo-miR-18a, ggo-miR-18b, ggo-miR-190a, ggo-miR-190b, ggo-miR-191, ggo-miR-192, ggo-miR-193a, ggo-miR-193b, ggo-miR-194, ggo-miR-195, ggo-miR-196, ggo-miR-196b, ggo-miR-197, ggo-miR-198, ggo-miR-199a, ggo-miR-19a, ggo-miR-19b, ggo-miR-200a, ggo-miR-200b, ggo-miR-200c, ggo-miR-202, ggo-miR-203b, ggo-miR-204, ggo-miR-205, ggo-miR-206, ggo-miR-20a, ggo-miR-20b, ggo-miR-21, ggo-miR-210, ggo-miR-2110, ggo-miR-214, ggo-miR-215, ggo-miR-216, ggo-miR-217, ggo-miR-218, ggo-miR-219, ggo-miR-22, ggo-miR-221, ggo-miR-222, ggo-miR-223, ggo-miR-224, ggo-miR-2355, ggo-miR-23a, ggo-miR-23b, ggo-miR-23c, ggo-miR-24, ggo-miR-25, ggo-miR-26a, ggo-miR-26b, ggo-miR-27a, ggo-miR-27b, ggo-miR-28, ggo-miR-296, ggo-miR-299, ggo-miR-29a, ggo-miR-29b, ggo-miR-29c, ggo-miR-301a, ggo-miR-30a-3p, ggo-miR-30a-5p, ggo-miR-30b, ggo-miR-30c, ggo-miR-30d, ggo-miR-30e, ggo-miR-31, ggo-miR-3126, ggo-miR-3127, ggo-miR-3129, ggo-miR-3138, ggo-miR-3151, ggo-miR-3173, ggo-miR-32, ggo-miR-320a, ggo-miR-320a-2, ggo-miR-320b, ggo-miR-323a, ggo-miR-323b, ggo-miR-324, ggo-miR-326, ggo-miR-328, ggo-miR-329b, ggo-miR-33, ggo-miR-330, ggo-miR-331, ggo-miR-335, ggo-miR-337, ggo-miR-338, ggo-miR-339, ggo-miR-340, ggo-miR-342, ggo-miR-345, ggo-miR-346, ggo-miR-34a, ggo-miR-34b, ggo-miR-34c, ggo-miR-3605, ggo-miR-361, ggo-miR-3612, ggo-miR-3615, ggo-miR-362, ggo-miR-363, ggo-miR-365a, ggo-miR-369, ggo-miR-370, ggo-miR-371b, ggo-miR-374a, ggo-miR-374b, ggo-miR-375, ggo-miR-376a, ggo-miR-376b, ggo-miR-376c, ggo-miR-377, ggo-miR-378a, ggo-miR-378b, ggo-miR-378e, ggo-miR-379, ggo-miR-380, ggo-miR-381, ggo-miR-382, ggo-miR-383, ggo-miR-3923, ggo-miR-3927, ggo-miR-3943, ggo-miR-409, ggo-miR-410, ggo-miR-411, ggo-miR-421, ggo-miR-423, ggo-miR-424, ggo-miR-429, ggo-miR-431, ggo-miR-432, ggo-miR-433, ggo-miR-4429, ggo-miR-4446, ggo-miR-448, ggo-miR-4484, ggo-miR-4488, ggo-miR-450a, ggo-miR-450b, ggo-miR-4510, ggo-miR-452, ggo-miR-4520b, ggo-miR-4524a, ggo-miR-4529, ggo-miR-4536, ggo-miR-454, ggo-miR-455, ggo-miR-4660, ggo-miR-4738, ggo-miR-4743, ggo-miR-483, ggo-miR-484, ggo-miR-485, ggo-miR-486, ggo-miR-487a, ggo-miR-487b, ggo-miR-488, ggo-miR-490, ggo-miR-491, ggo-miR-493, ggo-miR-494, ggo-miR-495, ggo-miR-496, ggo-miR-497, ggo-miR-499a, ggo-miR-502a, ggo-miR-502b, ggo-miR-503, ggo-miR-504, ggo-miR-505, ggo-miR-508, ggo-miR-509, ggo-miR-515-3p, ggo-miR-515-5p, ggo-miR-516a, ggo-miR-516a-3p, ggo-miR-516a-5p, ggo-miR-516b, ggo-miR-517c, ggo-miR-517c-3p, ggo-miR-517c-5p, ggo-miR-518b, ggo-miR-518c-3p, ggo-miR-518c-5p, ggo-miR-518d-3p, ggo-miR-518d-5p, ggo-miR-518e-3p, ggo-miR-518e-5p, ggo-miR-518f-3p, ggo-miR-518f-5p, ggo-miR-519a, ggo-miR-519a-3p, ggo-miR-519a-5p, ggo-miR-519b, ggo-miR-519c, ggo-miR-519c-3p, ggo-miR-519c-5p, ggo-miR-519d, ggo-miR-519e-3p, ggo-miR-519e-5p, ggo-miR-520c, ggo-miR-520f, ggo-miR-520g, ggo-miR-521, ggo-miR-522-3p, ggo-miR-522-5p, ggo-miR-523-3p, ggo-miR-523-5p, ggo-miR-524-3p, ggo-miR-524-5p, ggo-miR-525-3p, ggo-miR-525-5p, ggo-miR-526a, ggo-miR-532, ggo-miR-539, ggo-miR-541, ggo-miR-542, ggo-miR-543, ggo-miR-548a, ggo-miR-548b, ggo-miR-548c, ggo-miR-548d, ggo-miR-548e, ggo-miR-548f, ggo-miR-551a, ggo-miR-551b, ggo-miR-556, ggo-miR-561, ggo-miR-574, ggo-miR-577, ggo-miR-582, ggo-miR-584, ggo-miR-589, ggo-miR-590, ggo-miR-592, ggo-miR-598, ggo-miR-609, ggo-miR-615, ggo-miR-624, ggo-miR-628, ggo-miR-629, ggo-miR-641, ggo-miR-651, ggo-miR-652, ggo-miR-654, ggo-miR-655, ggo-miR-656, ggo-miR-660, ggo-miR-668, ggo-miR-671, ggo-miR-675b, ggo-miR-676, ggo-miR-7, ggo-miR-758, ggo-miR-760, ggo-miR-766, ggo-miR-767, ggo-miR-769, ggo-miR-873, ggo-miR-874, ggo-miR-877, ggo-miR-885, ggo-miR-887, ggo-miR-889, ggo-miR-9, ggo-miR-92, ggo-miR-92b, ggo-miR-93, ggo-miR-940, ggo-miR-95, ggo-miR-96, ggo-miR-98, ggo-miR-99a, ghb-miR169a, ghr-miR156a, ghr-miR156b, ghr-miR156c, ghr-miR156d, ghr-miR160, ghr-miR162a, ghr-miR164, ghr-miR166b, ghr-miR167a, ghr-miR167b, ghr-miR169a, ghr-miR169b, ghr-miR172, ghr-miR2948-5p, ghr-miR2949a-3p, ghr-miR2949a-5p, ghr-miR2949b, ghr-miR2949c, ghr-miR2950, ghr-miR3476-3p, ghr-miR3476-5p, ghr-miR390a, ghr-miR390b, ghr-miR390c, ghr-miR393, ghr-miR394a, ghr-miR394b, ghr-miR396a, ghr-miR396b, ghr-miR398, ghr-miR399a, ghr-miR399b, ghr-miR399c, ghr-miR399d, ghr-miR399e, ghr-miR479, ghr-miR482a, ghr-miR482b, ghr-miR7484a, ghr-miR7484b, ghr-miR7485, ghr-miR7486a, ghr-miR7486b, ghr-miR7487, ghr-miR7488, ghr-miR7489, ghr-miR7490, ghr-miR7491, ghr-miR7492a, ghr-miR7492b, ghr-miR7492c, ghr-miR7493, ghr-miR7494, ghr-miR7495a, ghr-miR7495b, ghr-miR7496a, ghr-miR7496b, ghr-miR7497, ghr-miR7498, ghr-miR7499, ghr-miR7500, ghr-miR7501, ghr-miR7502, ghr-miR7503, ghr-miR7504a, ghr-miR7504b, ghr-miR7505, ghr-miR7506, ghr-miR7507, ghr-miR7508, ghr-miR7509, ghr-miR7510a, ghr-miR7510b, ghr-miR7511, ghr-miR7512, ghr-miR7513, ghr-miR7514, ghr-miR827a, ghr-miR827b, ghr-miR827c, gma-miR10186a, gma-miR10186b, gma-miR10186c, gma-miR10186d, gma-miR10186e, gma-miR10186f, gma-miR10186g, gma-miR10186h, gma-miR10186i, gma-miR10186j, gma-miR10186k, gma-miR101861, gma-miR10187, gma-miR10188, gma-miR10189, gma-miR10190, gma-miR10191, gma-miR10192, gma-miR10193a, gma-miR10193b, gma-miR10193c, gma-miR10193d, gma-miR10193e, gma-miR10193f, gma-miR10194, gma-miR10195, gma-miR10196, gma-miR10197, gma-miR10198, gma-miR10199, gma-miR10200, gma-miR10201, gma-miR10405a, gma-miR10405b, gma-miR10405c, gma-miR10405d, gma-miR10405e, gma-miR10406a, gma-miR10406b, gma-miR10407a, gma-miR10407b, gma-miR10407c, gma-miR10407d, gma-miR10408, gma-miR10409, gma-miR10410a, gma-miR10410b, gma-miR10410c, gma-miR10411, gma-miR10412, gma-miR10413a, gma-miR10413b, gma-miR10414, gma-miR10415, gma-miR10416, gma-miR10417, gma-miR10418, gma-miR10419, gma-miR10420, gma-miR10421, gma-miR10422, gma-miR10423, gma-miR10424a, gma-miR10424b, gma-miR10424c, gma-miR10424d, gma-miR10425, gma-miR10426, gma-miR10427a, gma-miR10427b, gma-miR10428, gma-miR10429, gma-miR10430, gma-miR10431, gma-miR10432, gma-miR10433, gma-miR10434, gma-miR10435, gma-miR10436, gma-miR10437, gma-miR10438, gma-miR10439, gma-miR10440, gma-miR10441, gma-miR10442, gma-miR10443, gma-miR10444, gma-miR10445, gma-miR10446, gma-miR10447, gma-miR10448, gma-miR1446, gma-miR1507a, gma-miR1507b, gma-miR1507c-3p, gma-miR1507c-5p, gma-miR1508a, gma-miR1508b, gma-miR1508c, gma-miR1509a, gma-miR1509b, gma-miR1510a-3p, gma-miR1510a-5p, gma-miR1510b-3p, gma-miR1510b-5p, gma-miR1511, gma-miR1512a-3p, gma-miR1512a-5p, gma-miR1512b, gma-miR1512c, gma-miR1513a-3p, gma-miR1513a-5p, gma-miR1513b, gma-miR1513c, gma-miR1514a-3p, gma-miR1514a-5p, gma-miR1514b-3p, gma-miR1514b-5p, gma-miR1515a, gma-miR1515b, gma-miR1516a-3p, gma-miR1516a-5p, gma-miR1516b, gma-miR1516c, gma-miR1516d, gma-miR1517, gma-miR1518, gma-miR1519, gma-miR1520a, gma-miR1520b, gma-miR1520c, gma-miR1520d, gma-miR1520e, gma-miR1520f-3p, gma-miR1520f-5p, gma-miR1520g, gma-miR1520h, gma-miR1520i, gma-miR1520j, gma-miR1520k, gma-miR15201, gma-miR1520m, gma-miR1520n, gma-miR15200, gma-miR1520p, gma-miR1520q, gma-miR1520r, gma-miR1521a, gma-miR1521b, gma-miR1522, gma-miR1523a, gma-miR1523b, gma-miR1524, gma-miR1525, gma-miR1526, gma-miR1527, gma-miR1528, gma-miR1529, gma-miR1530, gma-miR1531-3p, gma-miR1531-5p, gma-miR1532, gma-miR1533, gma-miR1534, gma-miR1535a, gma-miR1535b, gma-miR1536, gma-miR156a, gma-miR156aa, gma-miR156ab, gma-miR156b, gma-miR156c, gma-miR156d, gma-miR156e, gma-miR156f, gma-miR156g, gma-miR156h, gma-miR156i, gma-miR156j, gma-miR156k, gma-miR1561, gma-miR156m, gma-miR156n, gma-miR1560, gma-miR156p, gma-miR156q, gma-miR156r, gma-miR156s, gma-miR156t, gma-miR156u, gma-miR156v, gma-miR156w, gma-miR156x, gma-miR156y, gma-miR156z, gma-miR159a-3p, gma-miR159a-5p, gma-miR159b-3p, gma-miR159b-5p, gma-miR159c, gma-miR159d, gma-miR159e-3p, gma-miR159e-5p, gma-miR159f-3p, gma-miR159f-5p, gma-miR160a-3p, gma-miR160a-5p, gma-miR160b, gma-miR160c, gma-miR160d, gma-miR160e, gma-miR160f, gma-miR162a, gma-miR162b, gma-miR162c, gma-miR164a, gma-miR164b, gma-miR164c, gma-miR164d, gma-miR164e, gma-miR164f, gma-miR164g, gma-miR164h, gma-miR164i, gma-miR164j, gma-miR164k, gma-miR166a-3p, gma-miR166a-5p, gma-miR166b, gma-miR166c-3p, gma-miR166c-5p, gma-miR166d, gma-miR166e, gma-miR166f, gma-miR166g, gma-miR166 h-3p, gma-miR166 h-5p, gma-miR166i-3p, gma-miR166i-5p, gma-miR166j-3p, gma-miR166j-5p, gma-miR166k, gma-miR1661, gma-miR166m, gma-miR166n, gma-miR1660, gma-miR166p, gma-miR166q, gma-miR166r, gma-miR166s, gma-miR166t, gma-miR166u, gma-miR167a, gma-miR167b, gma-miR167c, gma-miR167d, gma-miR167e, gma-miR167f, gma-miR167g, gma-miR167h, gma-miR167i, gma-miR167j, gma-miR167k, gma-miR1671, gma-miR168a, gma-miR168b, gma-miR169a, gma-miR169b, gma-miR169c, gma-miR169d, gma-miR169e, gma-miR169f, gma-miR169g, gma-miR169h, gma-miR169i-3p, gma-miR169i-5p, gma-miR169j-3p, gma-miR169j-5p, gma-miR169k, gma-miR1691-3p, gma-miR1691-5p, gma-miR169m, gma-miR169n-3p, gma-miR169n-5p, gma-miR1690, gma-miR169p, gma-miR169r, gma-miR169s-3p, gma-miR169s-5p, gma-miR169t, gma-miR169u, gma-miR169v, gma-miR169w, gma-miR171a, gma-miR171b-3p, gma-miR171b-5p, gma-miR171c-3p, gma-miR171c-5p, gma-miR171d, gma-miR171e, gma-miR171f, gma-miR171g, gma-miR171h, gma-miR171i-3p, gma-miR171i-5p, gma-miR171j-3p, gma-miR171j-5p, gma-miR171k-3p, gma-miR171k-5p, gma-miR1711, gma-miR171m, gma-miR171n, gma-miR1710-3p, gma-miR1710-5p, gma-miR171p, gma-miR171q, gma-miR171r, gma-miR171s, gma-miR171t, gma-miR171u, gma-miR172a, gma-miR172b-3p, gma-miR172b-5p, gma-miR172c, gma-miR172d, gma-miR172e, gma-miR172f, gma-miR172g, gma-miR172 h-3p, gma-miR172h-5p, gma-miR172i-3p, gma-miR172i-5p, gma-miR172j, gma-miR172k, gma-miR1721, gma-miR2107, gma-miR2108a, gma-miR2108b, gma-miR2109-3p, gma-miR2109-5p, gma-miR2111a, gma-miR2111b, gma-miR2111c, gma-miR2111d, gma-miR21Ile, gma-miR2111f, gma-miR2118a-3p, gma-miR2118a-5p, gma-miR2118b-3p, gma-miR2118b-5p, gma-miR2119, gma-miR2606a, gma-miR2606b, gma-miR319a, gma-miR319b, gma-miR319c, gma-miR319d, gma-miR319e, gma-miR319f, gma-miR319g, gma-miR319h, gma-miR319i, gma-miR319j, gma-miR319k, gma-miR3191, gma-miR319m, gma-miR319n, gma-miR3190, gma-miR319p, gma-miR319q, gma-miR3522, gma-miR390a-3p, gma-miR390a-5p, gma-miR390b-3p, gma-miR390b-5p, gma-miR390c, gma-miR390d, gma-miR390e, gma-miR390f, gma-miR390g, gma-miR391-5p, gma-miR391a-3p, gma-miR393a, gma-miR393b, gma-miR393c-3p, gma-miR393c-5p, gma-miR393d, gma-miR393e, gma-miR393f, gma-miR393g, gma-miR393h, gma-miR393i, gma-miR393j, gma-miR393k, gma-miR394a-3p, gma-miR394a-5p, gma-miR394b-3p, gma-miR394b-5p, gma-miR394c-5p, gma-miR394d, gma-miR394e, gma-miR394f, gma-miR394g, gma-miR395a, gma-miR395b, gma-miR395c, gma-miR395d, gma-miR395e, gma-miR395f, gma-miR395g, gma-miR395h, gma-miR395i, gma-miR395j, gma-miR395k, gma-miR3951, gma-miR395m, gma-miR396a-3p, gma-miR396a-5p, gma-miR396b-3p, gma-miR396b-5p, gma-miR396c, gma-miR396d, gma-miR396e, gma-miR396f, gma-miR396g, gma-miR396h, gma-miR396i-3p, gma-miR396i-5p, gma-miR396j, gma-miR396k-3p, gma-miR396k-5p, gma-miR397a, gma-miR397b-3p, gma-miR397b-5p, gma-miR398a, gma-miR398b-3p, gma-miR398b-5p, gma-miR398c, gma-miR398d, gma-miR399a, gma-miR399b, gma-miR399c, gma-miR399d, gma-miR399e, gma-miR399f, gma-miR399g, gma-miR399h, gma-miR399i, gma-miR399j, gma-miR399k, gma-miR3991, gma-miR399m, gma-miR399n, gma-miR3990, gma-miR403a, gma-miR403b, gma-miR408a-3p, gma-miR408a-5p, gma-miR408b-3p, gma-miR408b-5p, gma-miR408c-3p, gma-miR408c-5p, gma-miR408d, gma-miR4340, gma-miR4341, gma-miR4342, gma-miR4343a, gma-miR4343b, gma-miR4344, gma-miR4345, gma-miR4346, gma-miR4347, gma-miR4348a-3p, gma-miR4348a-5p, gma-miR4348b, gma-miR4348c, gma-miR4348d, gma-miR4349-3p, gma-miR4349-5p, gma-miR4350, gma-miR4351, gma-miR4352a, gma-miR4352b, gma-miR4353, gma-miR4354, gma-miR4355, gma-miR4356, gma-miR4357, gma-miR4358, gma-miR4359a, gma-miR4359b, gma-miR4360, gma-miR4361, gma-miR4362, gma-miR4363, gma-miR4364a, gma-miR4364b, gma-miR4365, gma-miR4366, gma-miR4367, gma-miR4368a, gma-miR4368b, gma-miR4369, gma-miR4370, gma-miR4371a, gma-miR4371b, gma-miR4371c, gma-miR4372a, gma-miR4372b, gma-miR4373, gma-miR4374a, gma-miR4374b, gma-miR4374c, gma-miR4375, gma-miR4377, gma-miR4378a, gma-miR4378b, gma-miR4379, gma-miR4380a, gma-miR4380b, gma-miR4381, gma-miR4382, gma-miR4383, gma-miR4384, gma-miR4385, gma-miR4386, gma-miR4387a, gma-miR4387b, gma-miR4387c, gma-miR4387d, gma-miR4387e, gma-miR4388, gma-miR4389, gma-miR4390, gma-miR4391, gma-miR4392, gma-miR4392b, gma-miR4393a, gma-miR4393b, gma-miR4394, gma-miR4395, gma-miR4396, gma-miR4397-3p, gma-miR4397-5p, gma-miR4398, gma-miR4399, gma-miR4400, gma-miR4401a, gma-miR4401b, gma-miR4402, gma-miR4403, gma-miR4404, gma-miR4405, gma-miR4405b, gma-miR4406, gma-miR4407, gma-miR4408, gma-miR4409, gma-miR4410, gma-miR4411, gma-miR4412-3p, gma-miR4412-5p, gma-miR4413a, gma-miR4413b, gma-miR4414a-3p, gma-miR4414a-5p, gma-miR4414b, gma-miR4415a-3p, gma-miR4415a-5p, gma-miR4415b-3p, gma-miR4415b-5p, gma-miR4416a, gma-miR4416b, gma-miR4416c-3p, gma-miR4416c-5p, gma-miR482a-3p, gma-miR482a-5p, gma-miR482b-3p, gma-miR482b-5p, gma-miR482c-3p, gma-miR482c-5p, gma-miR482d-3p, gma-miR482d-5p, gma-miR482e, gma-miR4992, gma-miR4993, gma-miR4994-3p, gma-miR4994-5p, gma-miR4995, gma-miR4996, gma-miR4997, gma-miR4997b, gma-miR4998, gma-miR5030, gma-miR5030b, gma-miR5031, gma-miR5032, gma-miR5033, gma-miR5034, gma-miR5035-3p, gma-miR5035-5p, gma-miR5036, gma-miR5037a, gma-miR5037b, gma-miR5037c, gma-miR5037d, gma-miR5038a, gma-miR5038b, gma-miR5039, gma-miR5040, gma-miR5041-3p, gma-miR5041-5p, gma-miR5042-3p, gma-miR5042-5p, gma-miR5043, gma-miR5044, gma-miR5225, gma-miR530a, gma-miR530b, gma-miR530c, gma-miR530d, gma-miR530e, gma-miR5368, gma-miR5369, gma-miR5370, gma-miR5371-3p, gma-miR5371-5p, gma-miR5372, gma-miR5373, gma-miR5374-3p, gma-miR5374-5p, gma-miR5375, gma-miR5376, gma-miR5377, gma-miR5377b, gma-miR5378, gma-miR5379, gma-miR5380a, gma-miR5380b, gma-miR5380c, gma-miR5559, gma-miR5667-3p, gma-miR5667-5p, gma-miR5668, gma-miR5669, gma-miR5670a, gma-miR5670b, gma-miR5671a, gma-miR5671b, gma-miR5672, gma-miR5673, gma-miR5674a, gma-miR5674b, gma-miR5675, gma-miR5676, gma-miR5677, gma-miR5678, gma-miR5679, gma-miR5761a, gma-miR5761b, gma-miR5762, gma-miR5763a, gma-miR5763b, gma-miR5763c, gma-miR5764, gma-miR5764b, gma-miR5765, gma-miR5766, gma-miR5767, gma-miR5768, gma-miR5769, gma-miR5770a, gma-miR5770b, gma-miR5770c, gma-miR5771, gma-miR5772, gma-miR5773, gma-miR5774a, gma-miR5774b, gma-miR5775, gma-miR5776, gma-miR5777, gma-miR5778, gma-miR5779, gma-miR5780a, gma-miR5780b, gma-miR5780c, gma-miR5780d, gma-miR5781, gma-miR5782, gma-miR5783, gma-miR5784, gma-miR5785, gma-miR5786, gma-miR6299, gma-miR6300, gma-miR828a, gma-miR828b, gma-miR862a, gma-miR862b, gma-miR9722, gma-miR9723, gma-miR9724, gma-miR9724b, gma-miR9725, gma-miR9726, gma-miR9727, gma-miR9728, gma-miR9729, gma-miR9730, gma-miR9731, gma-miR9732, gma-miR9733, gma-miR9734, gma-miR9735, gma-miR9736, gma-miR9737, gma-miR9738, gma-miR9739, gma-miR9740, gma-miR9741, gma-miR9742, gma-miR9743, gma-miR9744, gma-miR9745, gma-miR9746a, gma-miR9746b, gma-miR9746c, gma-miR9746d, gma-miR9746e, gma-miR9746f, gma-miR9746g, gma-miR9746h, gma-miR9746i, gma-miR9747, gma-miR9748, gma-miR9749, gma-miR9750-3p, gma-miR9750-5p, gma-miR9751, gma-miR9752, gma-miR9753, gma-miR9754, gma-miR9755, gma-miR9756, gma-miR9757, gma-miR9758, gma-miR9759, gma-miR9760, gma-miR9761, gma-miR9762, gma-miR9763, gma-miR9764, gma-miR9765, gma-miR9766, gma-miR9767, gmo-let-7a-3-3p, gmo-let-7a-3p, gmo-let-7a-4-3p, gmo-let-7a-5-3p, gmo-let-7a-5p, gmo-let-7c-3p, gmo-let-7c-5p, gmo-let-7d-3p, gmo-let-7d-5p, gmo-let-7e-3p, gmo-let-7e-5p, gmo-let-7f-3p, gmo-let-7f-5p, gmo-let-7g-3p, gmo-let-7g-5p, gmo-let-7 h-2-3p, gmo-let-7 h-3p, gmo-let-7 h-5p, gmo-let-7i-2-3p, gmo-let-7i-3p, gmo-let-7i-5p, gmo-let-7j-3p, gmo-let-7j-5p, gmo-miR-1-2-5p, gmo-miR-1-3p, gmo-miR-1-5p, gmo-miR-100a-3p, gmo-miR-100a-5p, gmo-miR-100b-5p, gmo-miR-101a-3p, gmo-miR-101a-5p, gmo-miR-101b-3p, gmo-miR-101b-5p, gmo-miR-103-2-5p, gmo-miR-103-3p, gmo-miR-103-5p, gmo-miR-10544-3p, gmo-miR-10544-5p, gmo-miR-10545-3p, gmo-miR-10545-5p, gmo-miR-10585-5p, gmo-miR-10a-3p, gmo-miR-10a-5p, gmo-miR-10b-5p, gmo-miR-10c-3p, gmo-miR-10c-5p, gmo-miR-10d-3p, gmo-miR-10d-5p, gmo-miR-11183-3p, gmo-miR-11183-5p, gmo-miR-11184a-3p, gmo-miR-11184b-5p, gmo-miR-11185-5p, gmo-miR-11186-5p, gmo-miR-11187-3p, gmo-miR-11187-5p, gmo-miR-11188-3p, gmo-miR-11189-5p, gmo-miR-11190-5p, gmo-miR-11191-3p, gmo-miR-11192-5p, gmo-miR-11193-5p, gmo-miR-11194-5p, gmo-miR-11195-5p, gmo-miR-11196-3p, gmo-miR-11197-3p, gmo-miR-11198-5p, gmo-miR-11199-5p, gmo-miR-11200-5p, gmo-miR-11201-3p, gmo-miR-11202-5p, gmo-miR-11203-5p, gmo-miR-11204a-3p, gmo-miR-11204b-3p, gmo-miR-11204c-3p, gmo-miR-11204d-3p, gmo-miR-11205-3p, gmo-miR-11205-5p, gmo-miR-11206-5p, gmo-miR-11207-3p, gmo-miR-11208-5p, gmo-miR-11209-3p, gmo-miR-11210-3p, gmo-miR-11210-5p, gmo-miR-11211-3p, gmo-miR-11212-5p, gmo-miR-11213-5p, gmo-miR-11214-5p, gmo-miR-11215-3p, gmo-miR-11216-5p, gmo-miR-11217-5p, gmo-miR-11218-5p, gmo-miR-11219-5p, gmo-miR-11220a-5p, gmo-miR-11220b-5p, gmo-miR-11221-3p, gmo-miR-11221-5p, gmo-miR-11222-5p, gmo-miR-11223-3p, gmo-miR-11223-5p, gmo-miR-11224a-5p, gmo-miR-11224b-3p, gmo-miR-11224b-5p, gmo-miR-11225-5p, gmo-miR-11226-3p, gmo-miR-11227-5p, gmo-miR-11228-3p, gmo-miR-11229-5p, gmo-miR-11230-5p, gmo-miR-11231-3p, gmo-miR-11232-3p, gmo-miR-11233-3p, gmo-miR-11234-3p, gmo-miR-11235-5p, gmo-miR-11236-3p, gmo-miR-11236-5p, gmo-miR-11237-5p, gmo-miR-11238-5p, gmo-miR-11239-3p, gmo-miR-11240-5p, gmo-miR-11241-3p, gmo-miR-11242-5p, gmo-miR-11243-3p, gmo-miR-11244-3p, gmo-miR-11245-5p, gmo-miR-11246-3p, gmo-miR-11247-5p, gmo-miR-11248-5p, gmo-miR-11249-3p, gmo-miR-11250-3p, gmo-miR-11251-3p, gmo-miR-11252-3p, gmo-miR-11252-5p, gmo-miR-11253-3p, gmo-miR-11254-3p, gmo-miR-11255-5p, gmo-miR-11256-5p, gmo-miR-11257-3p, gmo-miR-11258-5p, gmo-miR-11259-3p, gmo-miR-11259-5p, gmo-miR-11260-3p, gmo-miR-11260-5p, gmo-miR-11261-3p, gmo-miR-11262-3p, gmo-miR-11262-5p, gmo-miR-11263-3p, gmo-miR-11264-5p, gmo-miR-11265-3p, gmo-miR-11266a-5p, gmo-miR-11266b-3p, gmo-miR-11267-3p, gmo-miR-11267-5p, gmo-miR-11268-5p, gmo-miR-11269-5p, gmo-miR-11270-3p, gmo-miR-11271-5p, gmo-miR-11272-3p, gmo-miR-11272-5p, gmo-miR-11273-5p, gmo-miR-11274-5p, gmo-miR-11275-5p, gmo-miR-11276-5p, gmo-miR-11277-3p, gmo-miR-11278-5p, gmo-miR-11279-5p, gmo-miR-11280-3p, gmo-miR-11280-5p, gmo-miR-11281-3p, gmo-miR-11282-5p, gmo-miR-11929-3p, gmo-miR-11929-5p, gmo-miR-11930-3p, gmo-miR-11930-5p, gmo-miR-11931-2-3p, gmo-miR-11931-3p, gmo-miR-11931-5p, gmo-miR-11932-3p, gmo-miR-11932-5p, gmo-miR-11933-3p, gmo-miR-11933-5p, gmo-miR-11934-3p, gmo-miR-11934-5p, gmo-miR-11935-3p, gmo-miR-11935-5p, gmo-miR-11936-3p, gmo-miR-11936-5p, gmo-miR-11937-3p, gmo-miR-11937-5p, gmo-miR-122-3p, gmo-miR-122-5p, gmo-miR-124-3-5p, gmo-miR-124-3p, gmo-miR-124-5p, gmo-miR-125a-3p, gmo-miR-125a-5p, gmo-miR-125b-3p, gmo-miR-125b-5p, gmo-miR-126-3p, gmo-miR-126-5p, gmo-miR-128-2-5p, gmo-miR-128-3p, gmo-miR-128-5p, gmo-miR-129-3-5p, gmo-miR-129-3p, gmo-miR-129-4-5p, gmo-miR-129-5p, gmo-miR-130a-3p, gmo-miR-130a-5p, gmo-miR-130b-3p, gmo-miR-130b-5p, gmo-miR-132-2-5p, gmo-miR-132-3p, gmo-miR-132-5p, gmo-miR-1338-3p, gmo-miR-1338-5p, gmo-miR-133a-3p, gmo-miR-133a-5p, gmo-miR-133b-3p, gmo-miR-133b-5p, gmo-miR-135a-5p, gmo-miR-135b-3p, gmo-miR-135b-5p, gmo-miR-135c-3p, gmo-miR-135c-5p, gmo-miR-135d-3p, gmo-miR-135d-5p, gmo-miR-137-2-5p, gmo-miR-137-3p, gmo-miR-137-5p, gmo-miR-138-3-3p, gmo-miR-138-3p, gmo-miR-138-5p, gmo-miR-139-3p, gmo-miR-139-5p, gmo-miR-140-3p, gmo-miR-140-5p, gmo-miR-142-2-3p, gmo-miR-142-5p, gmo-miR-143-3p, gmo-miR-143-5p, gmo-miR-144-3p, gmo-miR-144-5p, gmo-miR-145-3p, gmo-miR-145-5p, gmo-miR-146-3p, gmo-miR-146-5p, gmo-miR-148-3p, gmo-miR-148-5p, gmo-miR-150-5p, gmo-miR-152-3p, gmo-miR-152-5p, gmo-miR-153a-3p, gmo-miR-153b-3p, gmo-miR-153b-5p, gmo-miR-155-5p, gmo-miR-15a-5p, gmo-miR-15b-5p, gmo-miR-16a-3p, gmo-miR-16a-5p, gmo-miR-16b-5p, gmo-miR-17-5p, gmo-miR-1788-3p, gmo-miR-1788-5p, gmo-miR-181a-3p, gmo-miR-181a-5p, gmo-miR-181b-3p, gmo-miR-181b-5p, gmo-miR-181c-3p, gmo-miR-181c-5p, gmo-miR-181d-5p, gmo-miR-182-3p, gmo-miR-182-5p, gmo-miR-183-3p, gmo-miR-183-5p, gmo-miR-184-3p, gmo-miR-184-5p, gmo-miR-187-3p, gmo-miR-187-5p, gmo-miR-18a-2-5p, gmo-miR-18a-3p, gmo-miR-18a-5p, gmo-miR-18b-5p, gmo-miR-190a-2-5p, gmo-miR-190a-3p, gmo-miR-190a-5p, gmo-miR-190b-3p, gmo-miR-190b-5p, gmo-miR-192-5p, gmo-miR-193a-3p, gmo-miR-193a-5p, gmo-miR-194a-5p, gmo-miR-194b-5p, gmo-miR-196a-5p, gmo-miR-196b-5p, gmo-miR-199-3p, gmo-miR-199-4-3p, gmo-miR-199-5p, gmo-miR-19a-2-5p, gmo-miR-19a-3p, gmo-miR-19a-5p, gmo-miR-19b-2-5p, gmo-miR-19b-3p, gmo-miR-19b-5p, gmo-miR-19c-5p, gmo-miR-19d-3p, gmo-miR-19d-5p, gmo-miR-200a-3p, gmo-miR-200a-5p, gmo-miR-200b-3p, gmo-miR-200b-5p, gmo-miR-202-3p, gmo-miR-203a-3p, gmo-miR-203a-5p, gmo-miR-203b-3p, gmo-miR-203b-5p, gmo-miR-204-5p, gmo-miR-205-2-3p, gmo-miR-205-3p, gmo-miR-205-5p, gmo-miR-206-3p, gmo-miR-206-5p, gmo-miR-20a-2-3p, gmo-miR-20a-3-3p, gmo-miR-20a-3p, gmo-miR-20a-5p, gmo-miR-20b-5p, gmo-miR-21-3p, gmo-miR-21-5p, gmo-miR-210-3p, gmo-miR-210-5p, gmo-miR-212-2-5p, gmo-miR-212-3p, gmo-miR-212-5p, gmo-miR-214-2-5p, gmo-miR-214-3p, gmo-miR-214-5p, gmo-miR-216a-3p, gmo-miR-216a-5p, gmo-miR-216b-5p, gmo-miR-217-3p, gmo-miR-217-5p, gmo-miR-2184-3p, gmo-miR-2184-5p, gmo-miR-2187a-5p, gmo-miR-2187b-3p, gmo-miR-2187b-5p, gmo-miR-2188-3p, gmo-miR-2188-5p, gmo-miR-218a-3p, gmo-miR-218a-5p, gmo-miR-218b-5p, gmo-miR-219-3-3p, gmo-miR-219-3p, gmo-miR-219-5p, gmo-miR-221-2-5p, gmo-miR-221-3p, gmo-miR-221-5p, gmo-miR-222-2-5p, gmo-miR-222-3p, gmo-miR-222-5p, gmo-miR-223a-3p, gmo-miR-223a-5p, gmo-miR-223b-3p, gmo-miR-22a-2-5p, gmo-miR-22a-3p, gmo-miR-22a-5p, gmo-miR-22b-3p, gmo-miR-22b-5p, gmo-miR-23a-3p, gmo-miR-23a-5p, gmo-miR-23b-3p, gmo-miR-24a-3-5p, gmo-miR-24a-3p, gmo-miR-24a-4-5p, gmo-miR-24a-5p, gmo-miR-24b-5p, gmo-miR-25-3p, gmo-miR-25-5p, gmo-miR-26a-2-3p, gmo-miR-26a-3p, gmo-miR-26a-4-3p, gmo-miR-26a-5p, gmo-miR-26b-5p, gmo-miR-27a-3p, gmo-miR-27a-5p, gmo-miR-27b-3p, gmo-miR-27b-5p, gmo-miR-27c-3p, gmo-miR-27c-5p, gmo-miR-27d-3p, gmo-miR-27d-5p, gmo-miR-29a-2-5p, gmo-miR-29a-3-5p, gmo-miR-29a-3p, gmo-miR-29a-5p, gmo-miR-29b-2-5p, gmo-miR-29b-3p, gmo-miR-29b-5p, gmo-miR-29c-3p, gmo-miR-29c-5p, gmo-miR-29d-3p, gmo-miR-29d-5p, gmo-miR-301a-3p, gmo-miR-301a-5p, gmo-miR-301b-3p, gmo-miR-301c-3p, gmo-miR-30a-3p, gmo-miR-30a-5p, gmo-miR-30b-3p, gmo-miR-30b-5p, gmo-miR-30c-3p, gmo-miR-30c-5p, gmo-miR-30d-3p, gmo-miR-30d-5p, gmo-miR-30e-3p, gmo-miR-30e-5p, gmo-miR-31-5p, gmo-miR-338-2-5p, gmo-miR-338-3-5p, gmo-miR-338-3p, gmo-miR-338-5p, gmo-miR-33b-2-3p, gmo-miR-33b-3p, gmo-miR-33b-5p, gmo-miR-34-3p, gmo-miR-34-5p, gmo-miR-363-3p, gmo-miR-365-3p, gmo-miR-365-5p, gmo-miR-375-3p, gmo-miR-375-5p, gmo-miR-429-3p, gmo-miR-429-5p, gmo-miR-430a-3p, gmo-miR-430b-5p, gmo-miR-430c-5p, gmo-miR-430d-5p, gmo-miR-430e-3p, gmo-miR-430e-5p, gmo-miR-430f-3p, gmo-miR-430g-3p, gmo-miR-449a-3p, gmo-miR-449a-5p, gmo-miR-449b-5p, gmo-miR-451-3p, gmo-miR-451-5p, gmo-miR-454-3p, gmo-miR-455-3p, gmo-miR-455-5p, gmo-miR-456-3p, gmo-miR-456-5p, gmo-miR-458-3p, gmo-miR-458-5p, gmo-miR-459-3p, gmo-miR-459-5p, gmo-miR-460-3p, gmo-miR-460-5p, gmo-miR-462-3p, gmo-miR-462-5p, gmo-miR-489-3p, gmo-miR-489-5p, gmo-miR-499-3p, gmo-miR-499-5p, gmo-miR-551-3p, gmo-miR-7132a-3p, gmo-miR-7132a-5p, gmo-miR-7132b-3p, gmo-miR-7132b-5p, gmo-miR-7133-3p, gmo-miR-722-3p, gmo-miR-722-5p, gmo-miR-723-3p, gmo-miR-723-5p, gmo-miR-724-3p, gmo-miR-724-5p, gmo-miR-725-3p, gmo-miR-725-5p, gmo-miR-727-5p, gmo-miR-727b-3p, gmo-miR-727b-5p, gmo-miR-728a-3p, gmo-miR-728b-3p, gmo-miR-730-5p, gmo-miR-731-5p, gmo-miR-734-3p, gmo-miR-736-3p, gmo-miR-736-5p, gmo-miR-737-3p, gmo-miR-737-5p, gmo-miR-7550-5p, gmo-miR-7552-3p, gmo-miR-7552-5p, gmo-miR-7a-5p, gmo-miR-7b-5p, gmo-miR-7c-5p, gmo-miR-8159-5p, gmo-miR-8160a-3p, gmo-miR-8160a-5p, gmo-miR-8160b-3p, gmo-miR-8160b-5p, gmo-miR-8825-5p, gmo-miR-9-3-3p, gmo-miR-9-3p, gmo-miR-9-5-3p, gmo-miR-9-5p, gmo-miR-9-6-3p, gmo-miR-92-2-5p, gmo-miR-92-3p, gmo-miR-92-5p, gmo-miR-93-3p, gmo-miR-93-5p, gmo-miR-96-3p, gmo-miR-96-5p, gmo-miR-99-3p, gmo-miR-99-5p, gpy-miR-5045, gra-miR1446, gra-miR157a, gra-miR157b, gra-miR164a, gra-miR166c, gra-miR166d, gra-miR167a, gra-miR167b, gra-miR167c, gra-miR172a, gra-miR172b, gra-miR3267, gra-miR3476, gra-miR398, gra-miR399, gra-miR477, gra-miR482, gra-miR482c, gra-miR482d, gra-miR5207, gra-miR530a, gra-miR530b, gra-miR5741, gra-miR7484c, gra-miR7484d, gra-miR7484e, gra-miR7484f, gra-miR7484g, gra-miR7484h, gra-miR7484i, gra-miR7484j, gra-miR7484k, gra-miR74841, gra-miR7484m, gra-miR7484n, gra-miR74840, gra-miR7486c, gra-miR7486d, gra-miR7486e, gra-miR7486f, gra-miR7486g, gra-miR7486h, gra-miR7486i, gra-miR7486j, gra-miR7492d, gra-miR7492e, gra-miR7492f, gra-miR7492g, gra-miR7492h, gra-miR7492i, gra-miR7492j, gra-miR7492k, gra-miR74921, gra-miR7492m, gra-miR7492n, gra-miR74920, gra-miR7493b, gra-miR7494b, gra-miR7494c, gra-miR7494d, gra-miR7494e, gra-miR7494f, gra-miR7502b, gra-miR7502c, gra-miR7502d, gra-miR7502e, gra-miR7502f, gra-miR7503b, gra-miR7504c, gra-miR7504d, gra-miR7504e, gra-miR7504f, gra-miR7504g, gra-miR7504h, gra-miR7504i, gra-miR7504j, gra-miR7504k, gra-miR75041, gra-miR7504m, gra-miR7504n, gra-miR7505b, gra-miR7506b, gra-miR7584e, gra-miR827b, gra-miR8632, gra-miR8633, gra-miR8634, gra-miR8635, gra-miR8636, gra-miR8637, gra-miR8638, gra-miR8639a, gra-miR8639b, gra-miR8639c, gra-miR8639d, gra-miR8639e, gra-miR8639f, gra-miR8639g, gra-miR8639h, gra-miR8639i, gra-miR8640, gra-miR8641, gra-miR8642, gra-miR8643a, gra-miR8643b, gra-miR8644, gra-miR8645, gra-miR8646, gra-miR8647, gra-miR8648, gra-miR8649, gra-miR8650, gra-miR8651, gra-miR8652, gra-miR8653a, gra-miR8653b, gra-miR8654a, gra-miR8654b, gra-miR8654c, gra-miR8655, gra-miR8656, gra-miR8657a, gra-miR8657b, gra-miR8657c, gra-miR8657d, gra-miR8657e, gra-miR8658, gra-miR8659a, gra-miR8659b, gra-miR8660, gra-miR8661, gra-miR8662, gra-miR8663a, gra-miR8663b, gra-miR8664, gra-miR8665, gra-miR8666, gra-miR8667, gra-miR8668, gra-miR8669, gra-miR8670a, gra-miR8670b, gra-miR8671, gra-miR8672, gra-miR8673, gra-miR8674a, gra-miR8674b, gra-miR8674c, gra-miR8675a, gra-miR8675b, gra-miR8675c, gra-miR8676, gra-miR8677, gra-miR8678, gra-miR8679, gra-miR8680, gra-miR8681, gra-miR8682, gra-miR8683, gra-miR8684, gra-miR8685, gra-miR8686, gra-miR8687, gra-miR8688, gra-miR8689, gra-miR8690, gra-miR8691, gra-miR8692, gra-miR8693, gra-miR8694a, gra-miR8694b, gra-miR8695, gra-miR8696, gra-miR8697, gra-miR8698, gra-miR8699, gra-miR8700, gra-miR8701, gra-miR8702, gra-miR8703a, gra-miR8703b, gra-miR8703c, gra-miR8704, gra-miR8705, gra-miR8706a, gra-miR8706b, gra-miR8707, gra-miR8708, gra-miR8709a, gra-miR8709b, gra-miR8709c, gra-miR8710a, gra-miR8710b, gra-miR8711, gra-miR8712, gra-miR8713, gra-miR8714, gra-miR8715, gra-miR8716, gra-miR8717, gra-miR8718, gra-miR8719, gra-miR8720, gra-miR8721a, gra-miR8721b, gra-miR8722, gra-miR8723a, gra-miR8723b, gra-miR8724, gra-miR8725, gra-miR8726, gra-miR8727, gra-miR8728, gra-miR8729, gra-miR8730, gra-miR8731, gra-miR8732, gra-miR8733, gra-miR8734, gra-miR8735, gra-miR8736, gra-miR8737, gra-miR8738a, gra-miR8738b, gra-miR8739, gra-miR8740, gra-miR8741, gra-miR8742a, gra-miR8742b, gra-miR8743a, gra-miR8743b, gra-miR8743c, gra-miR8743d, gra-miR8743e, gra-miR8744, gra-miR8745, gra-miR8746, gra-miR8747, gra-miR8748, gra-miR8749, gra-miR8750, gra-miR8751a, gra-miR8751b, gra-miR8752, gra-miR8753, gra-miR8754, gra-miR8755, gra-miR8756, gra-miR8757a, gra-miR8757b, gra-miR8758, gra-miR8759, gra-miR8760, gra-miR8761, gra-miR8762a, gra-miR8762b, gra-miR8762c, gra-miR8762d, gra-miR8762e, gra-miR8763, gra-miR8764, gra-miR8765, gra-miR8766, gra-miR8767a, gra-miR8767b, gra-miR8767c, gra-miR8768, gra-miR8769, gra-miR8770, gra-miR8771a, gra-miR8771b, gra-miR8771c, gra-miR8771d, gra-miR8771e, gra-miR8771f, gra-miR8772, gra-miR8773, gra-miR8774, gra-miR8775, gra-miR8776a, gra-miR8776b, gra-miR8776c, gra-miR8776d, gra-miR8777, gra-miR8778, gra-miR8779, gra-miR8780, gra-miR8781a, gra-miR8781b, gra-miR8782, gra-miR8783, gra-miR8784, gra-miR8785, gra-miR8786a, gra-miR8786b, gra-miR8787, gsa-bantam-3p, gsa-bantam-5p, gsa-let-7-3p, gsa-let-7-5p, gsa-lin-4-3p, gsa-lin-4-5p, gsa-miR-1-3p, gsa-miR-1-5p, gsa-miR-10a-3p, gsa-miR-10a-5p, gsa-miR-10b-3p, gsa-miR-10b-5p, gsa-miR-1175-3p, gsa-miR-1175-5p, gsa-miR-124a-3p, gsa-miR-124a-5p, gsa-miR-124b-3p, gsa-miR-124b-5p, gsa-miR-153-3p, gsa-miR-153-5p, gsa-miR-184a-3p, gsa-miR-184a-5p, gsa-miR-184b-3p, gsa-miR-184b-5p, gsa-miR-184c-3p, gsa-miR-184c-5p, gsa-miR-190-3p, gsa-miR-190-5p, gsa-miR-1992-3p, gsa-miR-1992-5p, gsa-miR-1993-3p, gsa-miR-1993-5p, gsa-miR-2001-3p, gsa-miR-2001-5p, gsa-miR-219-3p, gsa-miR-219-5p, gsa-miR-277a-3p, gsa-miR-277a-5p, gsa-miR-277b-3p, gsa-miR-277b-5p, gsa-miR-278-3p, gsa-miR-278-5p, gsa-miR-279-3p, gsa-miR-279-5p, gsa-miR-281-3p, gsa-miR-281-5p, gsa-miR-2a-3p, gsa-miR-2a-5p, gsa-miR-2b-3p, gsa-miR-2b-5p, gsa-miR-2c-3p, gsa-miR-2c-5p, gsa-miR-2d-3p, gsa-miR-2d-5p, gsa-miR-31-3p, gsa-miR-31-5p, gsa-miR-36-3p, gsa-miR-36-5p, gsa-miR-67-3p, gsa-miR-67-5p, gsa-miR-7-3p, gsa-miR-7-5p, gsa-miR-71a-3p, gsa-miR-71a-5p, gsa-miR-71b-3p, gsa-miR-71b-5p, gsa-miR-71c-3p, gsa-miR-71c-5p, gsa-miR-76a-3p, gsa-miR-76a-5p, gsa-miR-76b-3p, gsa-miR-76b-5p, gsa-miR-8-3p, gsa-miR-8-5p, gsa-miR-87-3p, gsa-miR-87-5p, gsa-miR-9-3p, gsa-miR-9-5p, gsa-miR-9389-3p, gsa-miR-9389-5p, gsa-miR-9390-3p, gsa-miR-9390-5p, gsa-miR-9391-3p, gsa-miR-9391-5p, gsa-miR-9392-3p, gsa-miR-9392-5p, gsa-miR-9393-3p, gsa-miR-9393-5p, gsa-miR-9394a-3p, gsa-miR-9394a-5p, gsa-miR-9394b-3p, gsa-miR-9394b-5p, gsa-miR-9395-3p, gsa-miR-9395-5p, gsa-miR-9396-3p, gsa-miR-9396-5p, gsa-miR-9397-3p, gsa-miR-9397-5p, gsa-miR-9398-3p, gsa-miR-9398-5p, gsa-miR-9399-3p, gsa-miR-9399-5p, gsa-miR-9400-3p, gsa-miR-9400-5p, gsa-miR-9401-3p, gsa-miR-9401-5p, gsa-miR-9402-3p, gsa-miR-9402-5p, gsa-miR-9403-3p, gsa-miR-9403-5p, gsa-miR-9404-3p, gsa-miR-9404-5p, gsa-miR-9405-3p, gsa-miR-9405-5p, gsa-miR-96a-3p, gsa-miR-96a-5p, gsa-miR-96b-3p, gsa-miR-96b-5p, gsa-miR-993-3p, gsa-miR-993-5p, gso-miR1507a, gso-miR1507b, gso-miR1508a, gso-miR1509a, gso-miR1510a, gso-miR1510b, gso-miR167a, gso-miR2109, gso-miR2218, gso-miR3522a, gso-miR3522b, gso-miR482a, gso-miR482b, han-miR156a, han-miR156b, han-miR156c, han-miR160a, han-miR3440, han-miR3630-3p, han-miR3630-5p, har-miR156a, har-miR156c, har-miR403a, hbr-miR156, hbr-miR159a, hbr-miR166a, hbr-miR166b, hbr-miR2118, hbr-miR319, hbr-miR396a, hbr-miR396b, hbr-miR398, hbr-miR408a, hbr-miR408b, hbr-miR476, hbr-miR482a, hbr-miR482b, hbr-miR6166, hbr-miR6167, hbr-miR6168, hbr-miR6169, hbr-miR6170, hbr-miR6171, hbr-miR6172, hbr-miR6173, hbr-miR6174, hbr-miR6175, hbr-miR6482, hbr-miR6483, hbr-miR6484, hbr-miR6485, hbr-miR9386, hbr-miR9387, hbv-miR-B14RC-3p, hbv-miR-B19-5p, hbv-miR-B2-3p, hbv-miR-B20-3p, hbv-miR-B20-5p, hbv-miR-B21RC-5p, hbv-miR-B22-3p, hbv-miR-B26-5p, hbv-miR-B2RC, hbv-miR-B3RC-3p, hbv-miR-B3RC-5p, hbv-miR-B4, hbv-miR-B7-5p, hbv-miR-B8-3p, hbv-miR-B8-5p, hci-miR156a, hci-miR156b, hci-miR164a, hcmv-miR-UL112-3p, hcmv-miR-UL112-5p, hcmv-miR-UL148D, hcmv-miR-UL22A-3p, hcmv-miR-UL22A-5p, hcmv-miR-UL36-3p, hcmv-miR-UL36-5p, hcmv-miR-UL59, hcmv-miR-UL69, hcmv-miR-UL70-3p, hcmv-miR-UL70-5p, hcmv-miR-US22-3p, hcmv-miR-US22-5p, hcmv-miR-US25-1-3p, hcmv-miR-US25-1-5p, hcmv-miR-US25-2-3p, hcmv-miR-US25-2-5p, hcmv-miR-US29-3p, hcmv-miR-US29-5p, hcmv-miR-US33-3p, hcmv-miR-US33-5p, hcmv-miR-US4-3p, hcmv-miR-US4-5p, hcmv-miR-US5-1, hcmv-miR-US5-2-3p, hcmv-miR-US5-2-5p, hco-lin-4, hco-miR-1, hco-miR-124, hco-miR-133, hco-miR-190-5p, hco-miR-2, hco-miR-2159-3p, hco-miR-228, hco-miR-234, hco-miR-235, hco-miR-236, hco-miR-242, hco-miR-250, hco-miR-252, hco-miR-255, hco-miR-259, hco-miR-265, hco-miR-277, hco-miR-307, hco-miR-40a, hco-miR-40b, hco-miR-40c, hco-miR-40d, hco-miR-40e, hco-miR-43, hco-miR-45, hco-miR-46, hco-miR-50, hco-miR-5352, hco-miR-55, hco-miR-5884, hco-miR-5885a, hco-miR-5885b, hco-miR-5885c, hco-miR-5886a, hco-miR-5886b, hco-miR-5887, hco-miR-5888, hco-miR-5889, hco-miR-5890a, hco-miR-5890b-3p, hco-miR-5890b-5p, hco-miR-5890c, hco-miR-5890d, hco-miR-5890e, hco-miR-5890f-3p, hco-miR-5890f-5p, hco-miR-5891, hco-miR-5892a, hco-miR-5892b, hco-miR-5893, hco-miR-5894a, hco-miR-5894b, hco-miR-5895, hco-miR-5896, hco-miR-5897a, hco-miR-5897b, hco-miR-5897c, hco-miR-5898-3p, hco-miR-5898-5p, hco-miR-5899, hco-miR-59, hco-miR-5900, hco-miR-5901, hco-miR-5902-3p, hco-miR-5902-5p, hco-miR-5903, hco-miR-5904a, hco-miR-5904b, hco-miR-5904c, hco-miR-5905, hco-miR-5906, hco-miR-5907, hco-miR-5908-3p, hco-miR-5908-5p, hco-miR-5909, hco-miR-5910, hco-miR-5911a, hco-miR-5911b, hco-miR-5911c, hco-miR-5912a, hco-miR-5912b, hco-miR-5913, hco-miR-5914, hco-miR-5915, hco-miR-5916, hco-miR-5917, hco-miR-5918a, hco-miR-5918b, hco-miR-5918c, hco-miR-5918d, hco-miR-5919, hco-miR-5920, hco-miR-5921-3p, hco-miR-5921-5p, hco-miR-5922, hco-miR-5923, hco-miR-5924-3p, hco-miR-5924-5p, hco-miR-5925, hco-miR-5926, hco-miR-5927, hco-miR-5928a, hco-miR-5928b, hco-miR-5928c, hco-miR-5928d, hco-miR-5928e, hco-miR-5929, hco-miR-5930, hco-miR-5931, hco-miR-5932, hco-miR-5933, hco-miR-5934, hco-miR-5935, hco-miR-5936, hco-miR-5937, hco-miR-5938, hco-miR-5939, hco-miR-5940, hco-miR-5941, hco-miR-5942, hco-miR-5943, hco-miR-5944, hco-miR-5945-3p, hco-miR-5945-5p, hco-miR-5946a, hco-miR-5946b, hco-miR-5946c, hco-miR-5947, hco-miR-5948, hco-miR-5949, hco-miR-5950, hco-miR-5951, hco-miR-5952, hco-miR-5953, hco-miR-5954, hco-miR-5955, hco-miR-5956, hco-miR-5957, hco-miR-5958, hco-miR-5959, hco-miR-5960, hco-miR-5961, hco-miR-5962, hco-miR-5963, hco-miR-5964, hco-miR-5965, hco-miR-5966, hco-miR-5967, hco-miR-5968, hco-miR-5969, hco-miR-5970, hco-miR-5971, hco-miR-5972, hco-miR-5973, hco-miR-5974, hco-miR-5975, hco-miR-5976, hco-miR-5977, hco-miR-5978, hco-miR-5979, hco-miR-5980, hco-miR-5981, hco-miR-5982, hco-miR-5983, hco-miR-5984a, hco-miR-5984b, hco-miR-5985, hco-miR-5986, hco-miR-5987, hco-miR-5988, hco-miR-5989, hco-miR-5990-5p, hco-miR-5991, hco-miR-60, hco-miR-61, hco-miR-63a, hco-miR-63b, hco-miR-7, hco-miR-71, hco-miR-72, hco-miR-76, hco-miR-79, hco-miR-790, hco-miR-83, hco-miR-84a, hco-miR-84b, hco-miR-86, hco-miR-87a, hco-miR-87b, hco-miR-87c, hco-miR-9, hco-miR-9551, hco-miR-993, hex-miR390a, hex-miR390b, hhi-let-7c, hhi-let-7j, hhi-miR-1, hhi-miR-10d, hhi-miR-129a, hhi-miR-129b-3p, hhi-miR-129b-5p, hhi-miR-147b, hhi-miR-15a, hhi-miR-1788, hhi-miR-181b, hhi-miR-182, hhi-miR-183, hhi-miR-187, hhi-miR-196, hhi-miR-199a, hhi-miR-21, hhi-miR-23a, hhi-miR-26, hhi-miR-301, hhi-miR-430a, hhi-miR-430b, hhi-miR-449, hhi-miR-723, hhi-miR-728, hhi-miR-737, hhi-miR-7788, hhi-miR-7789, hhi-miR-7790, hhi-miR-7791, hhi-miR-7792, hhi-miR-7793, hhi-miR-7794, hhi-miR-7795, hhi-miR-7796, hhi-miR-96, hhv6b-miR-Ro6-1-3p, hhv6b-miR-Ro6-1-5p, hhv6b-miR-Ro6-2-3p, hhv6b-miR-Ro6-2-5p, hhv6b-miR-Ro6-3-3p, hhv6b-miR-Ro6-3-5p, hhv6b-miR-Ro6-4-3p, hhv6b-miR-Ro6-4-5p, hiv1-miR-H1, hiv1-miR-N367, hiv1-miR-TAR-3p, hiv1-miR-TAR-5p, hma-miR-2022, hma-miR-2030, hma-miR-3002, hma-miR-3003a-3p, hma-miR-3003a-5p, hma-miR-3003b-3p, hma-miR-3003b-5p, hma-miR-3004, hma-miR-3005, hma-miR-3006, hma-miR-3007-3p, hma-miR-3007-5p, hma-miR-3008, hma-miR-3009, hma-miR-3010, hma-miR-3011, hma-miR-3012a, hma-miR-3012b, hma-miR-3013, hma-miR-3014, hme-bantam, hme-let-7, hme-miR-10, hme-miR-1000, hme-miR-11, hme-miR-1175, hme-miR-12, hme-miR-124, hme-miR-133, hme-miR-137, hme-miR-13a, hme-miR-13b, hme-miR-14, hme-miR-184, hme-miR-190, hme-miR-193-3p, hme-miR-193-5p, hme-miR-1a, hme-miR-1b, hme-miR-210, hme-miR-252, hme-miR-263a, hme-miR-263b, hme-miR-2733, hme-miR-2755, hme-miR-2756, hme-miR-276, hme-miR-2763, hme-miR-2765, hme-miR-2767, hme-miR-2768, hme-miR-277, hme-miR-278, hme-miR-2788-3p, hme-miR-2788-5p, hme-miR-2796, hme-miR-2797, hme-miR-279a, hme-miR-279b, hme-miR-279c, hme-miR-279d, hme-miR-281, hme-miR-282, hme-miR-283, hme-miR-285, hme-miR-2a, hme-miR-2b, hme-miR-2c, hme-miR-305, hme-miR-306, hme-miR-307, hme-miR-308, hme-miR-316, hme-miR-3286, hme-miR-33, hme-miR-3327, hme-miR-3338, hme-miR-34, hme-miR-6301-1-5p, hme-miR-6301-2-5p, hme-miR-6301-3p, hme-miR-6302-1-5p, hme-miR-6302-2-5p, hme-miR-6302-3p, hme-miR-6303-5p, hme-miR-6304-3p, hme-miR-6305-3p, hme-miR-6305-5p, hme-miR-6306-3p, hme-miR-6306-5p, hme-miR-6307-3p, hme-miR-6308-3p, hme-miR-6309-3p, hme-miR-6310-3p, hme-miR-6311-3p, hme-miR-6312-3p, hme-miR-6312-5p, hme-miR-7, hme-miR-71, hme-miR-745, hme-miR-79, hme-miR-8, hme-miR-87, hme-miR-927, hme-miR-928, hme-miR-929, hme-miR-92a, hme-miR-92b, hme-miR-932, hme-miR-965, hme-miR-970, hme-miR-971, hme-miR-981, hme-miR-993, hme-miR-998, hme-miR-9a, hme-miR-9b, hpa-miR156a, hpa-miR166a, hpa-miR171a, hpe-miR162a, hpe-miR166a, hpe-miR403a, hpo-let-7-3p, hpo-let-7-5p, hpo-lin-4-3p, hpo-lin-4-5p, hpo-miR-1-3p, hpo-miR-1-5p, hpo-miR-100-3p, hpo-miR-100-5p, hpo-miR-10021-3p, hpo-miR-10021-5p, hpo-miR-10022-3p, hpo-miR-10022-5p, hpo-miR-10023-3p, hpo-miR-10023-5p, hpo-miR-10024-3p, hpo-miR-10024-5p, hpo-miR-10025-3p, hpo-miR-10025-5p, hpo-miR-10026-3p, hpo-miR-10026-5p, hpo-miR-10027-3p, hpo-miR-10027-5p, hpo-miR-10028-3p, hpo-miR-10028-5p, hpo-miR-10029-3p, hpo-miR-10029-5p, hpo-miR-10030-3p, hpo-miR-10030-5p, hpo-miR-10031a-2-5p, hpo-miR-10031a-3p, hpo-miR-10031a-5p, hpo-miR-10031b-3p, hpo-miR-10031b-5p, hpo-miR-10031c-3p, hpo-miR-10031c-5p, hpo-miR-10031d-3p, hpo-miR-10031d-5p, hpo-miR-10031e-3p, hpo-miR-10031e-5p, hpo-miR-10032-3p, hpo-miR-10032-5p, hpo-miR-10033-3p, hpo-miR-10033-5p, hpo-miR-10034-3p, hpo-miR-10034-5p, hpo-miR-10035-3p, hpo-miR-10035-5p, hpo-miR-10036-2-5p, hpo-miR-10036-3p, hpo-miR-10036-5p, hpo-miR-10037-3p, hpo-miR-10037-5p, hpo-miR-10038-3p, hpo-miR-10038-5p, hpo-miR-10039-3p, hpo-miR-10039-5p, hpo-miR-10040-3p, hpo-miR-10040-5p, hpo-miR-10041-3p, hpo-miR-10041-5p, hpo-miR-10042-3p, hpo-miR-10042-5p, hpo-miR-10043a-3p, hpo-miR-10043a-5p, hpo-miR-10043b-3p, hpo-miR-10043b-5p, hpo-miR-10043c-3p, hpo-miR-10043c-5p, hpo-miR-10043d-3p, hpo-miR-10043d-5p, hpo-miR-10043e-3p, hpo-miR-10043e-5p, hpo-miR-10043f-3p, hpo-miR-10043f-5p, hpo-miR-10044-3p, hpo-miR-10044-5p, hpo-miR-10045-3p, hpo-miR-10045-5p, hpo-miR-10046-3p, hpo-miR-10046-5p, hpo-miR-10047-3p, hpo-miR-10047-5p, hpo-miR-10048-3p, hpo-miR-10048-5p, hpo-miR-10049-3p, hpo-miR-10049-5p, hpo-miR-10050-3p, hpo-miR-10050-5p, hpo-miR-10051-3p, hpo-miR-10051-5p, hpo-miR-10052-3p, hpo-miR-10052-5p, hpo-miR-10053-3p, hpo-miR-10053-5p, hpo-miR-10054-3p, hpo-miR-10054-5p, hpo-miR-10055-3p, hpo-miR-10055-5p, hpo-miR-10056-3p, hpo-miR-10056-5p, hpo-miR-10057-3p, hpo-miR-10057-5p, hpo-miR-10058-3p, hpo-miR-10058-5p, hpo-miR-10059-3p, hpo-miR-10059-5p, hpo-miR-10060-3p, hpo-miR-10060-5p, hpo-miR-10061-3p, hpo-miR-10061-5p, hpo-miR-10062-3p, hpo-miR-10062-5p, hpo-miR-10063a-3p, hpo-miR-10063a-5p, hpo-miR-10063b-3p, hpo-miR-10063b-5p, hpo-miR-10064-3p, hpo-miR-10064-5p, hpo-miR-10065a-3p, hpo-miR-10065a-5p, hpo-miR-10065b-2-3p, hpo-miR-10065b-3p, hpo-miR-10065b-5p, hpo-miR-10066-3p, hpo-miR-10066-5p, hpo-miR-10067-3p, hpo-miR-10067-5p, hpo-miR-10068-3p, hpo-miR-10068-5p, hpo-miR-10069-3p, hpo-miR-10069-5p, hpo-miR-10070-3p, hpo-miR-10070-5p, hpo-miR-10071-3p, hpo-miR-10071-5p, hpo-miR-10072a-3p, hpo-miR-10072a-5p, hpo-miR-10072b-3p, hpo-miR-10072b-5p, hpo-miR-10073-3p, hpo-miR-10073-5p, hpo-miR-10074-3p, hpo-miR-10074-5p, hpo-miR-10075a-3p, hpo-miR-10075a-5p, hpo-miR-10075b-3p, hpo-miR-10075b-5p, hpo-miR-10076-3p, hpo-miR-10076-5p, hpo-miR-10077-3p, hpo-miR-10077-5p, hpo-miR-10078-3p, hpo-miR-10078-5p, hpo-miR-10079-3p, hpo-miR-10079-5p, hpo-miR-10080-3p, hpo-miR-10080-5p, hpo-miR-10081-3p, hpo-miR-10081-5p, hpo-miR-10082-3p, hpo-miR-10082-5p, hpo-miR-10083-??, hpo-miR-10083-3p, hpo-miR-10084-3p, hpo-miR-10084-5p, hpo-miR-10085-3p, hpo-miR-10085-5p, hpo-miR-10086-3p, hpo-miR-10086-5p, hpo-miR-10087-3p, hpo-miR-10087-5p, hpo-miR-10088-3p, hpo-miR-10088-5p, hpo-miR-10089-3p, hpo-miR-10089-5p, hpo-miR-10090-3p, hpo-miR-10090-5p, hpo-miR-10091-3p, hpo-miR-10091-5p, hpo-miR-10092-3p, hpo-miR-10092-5p, hpo-miR-10093-3p, hpo-miR-10093-5p, hpo-miR-10094-3p, hpo-miR-10094-5p, hpo-miR-10095-3p, hpo-miR-10095-5p, hpo-miR-10096-3p, hpo-miR-10096-5p, hpo-miR-10097-3p, hpo-miR-10097-5p, hpo-miR-10098a-3p, hpo-miR-10098a-5p, hpo-miR-10098b-3p, hpo-miR-10098b-5p, hpo-miR-10099-3p, hpo-miR-10099-5p, hpo-miR-10100-3p, hpo-miR-10100-5p, hpo-miR-10101-3p, hpo-miR-10101-5p, hpo-miR-10102-3p, hpo-miR-10102-5p, hpo-miR-10103-3p, hpo-miR-10103-5p, hpo-miR-10104-3p, hpo-miR-10104-5p, hpo-miR-10105-3p, hpo-miR-10105-5p, hpo-miR-10106-3p, hpo-miR-10106-5p, hpo-miR-10107-3p, hpo-miR-10107-5p, hpo-miR-10108-3p, hpo-miR-10108-5p, hpo-miR-10109-3p, hpo-miR-10109-5p, hpo-miR-10110-3p, hpo-miR-10110-5p, hpo-miR-10111-3p, hpo-miR-10111-5p, hpo-miR-10112-3p, hpo-miR-10112-5p, hpo-miR-10113-3p, hpo-miR-10113-5p, hpo-miR-10114-3p, hpo-miR-10114-5p, hpo-miR-10115-3p, hpo-miR-10115-5p, hpo-miR-10116-3p, hpo-miR-10116-5p, hpo-miR-10117-3p, hpo-miR-10117-5p, hpo-miR-10118-3p, hpo-miR-10118-5p, hpo-miR-10119-3p, hpo-miR-10119-5p, hpo-miR-10120-3p, hpo-miR-10120-5p, hpo-miR-10121-3p, hpo-miR-10121-5p, hpo-miR-10122-3p, hpo-miR-10122-5p, hpo-miR-10123-3p, hpo-miR-10123-5p, hpo-miR-10124-3p, hpo-miR-10124-5p, hpo-miR-10125-3p, hpo-miR-10125-5p, hpo-miR-10126-3p, hpo-miR-10126-5p, hpo-miR-10127-3p, hpo-miR-10127-5p, hpo-miR-10128-3p, hpo-miR-10128-5p, hpo-miR-10129-3p, hpo-miR-10129-5p, hpo-miR-10130-3p, hpo-miR-10130-5p, hpo-miR-10131-3p, hpo-miR-10131-5p, hpo-miR-10132-3p, hpo-miR-10132-5p, hpo-miR-10133-3p, hpo-miR-10133-5p, hpo-miR-10134-2-5p, hpo-miR-10134-3p, hpo-miR-10134-5p, hpo-miR-10135-3p, hpo-miR-10135-5p, hpo-miR-10136-3p, hpo-miR-10136-5p, hpo-miR-10137a-3p, hpo-miR-10137a-5p, hpo-miR-10137b-3p, hpo-miR-10137b-5p, hpo-miR-10138-3p, hpo-miR-10138-5p, hpo-miR-10139-3p, hpo-miR-10139-5p, hpo-miR-10140-3p, hpo-miR-10140-5p, hpo-miR-10141-3p, hpo-miR-10141-5p, hpo-miR-10142-3p, hpo-miR-10142-5p, hpo-miR-10143-3p, hpo-miR-10143-5p, hpo-miR-10144-??, hpo-miR-10144-3p, hpo-miR-10145-3p, hpo-miR-10145-5p, hpo-miR-10146-3p, hpo-miR-10146-5p, hpo-miR-10147-3p, hpo-miR-10147-5p, hpo-miR-10148-3p, hpo-miR-10148-5p, hpo-miR-10149-3p, hpo-miR-10149-5p, hpo-miR-10150-3p, hpo-miR-10150-5p, hpo-miR-10151-3p, hpo-miR-10151-5p, hpo-miR-10152-3p, hpo-miR-10152-5p, hpo-miR-10153-3p, hpo-miR-10153-5p, hpo-miR-10154-3p, hpo-miR-10154-5p, hpo-miR-10155-3p, hpo-miR-10155-5p, hpo-miR-10156a-3p, hpo-miR-10156a-5p, hpo-miR-10156b-3p, hpo-miR-10156b-5p, hpo-miR-10157-3p, hpo-miR-10157-5p, hpo-miR-10158a-3p, hpo-miR-10158a-5p, hpo-miR-10158b-3p, hpo-miR-10158b-5p, hpo-miR-10159-3p, hpo-miR-10159-5p, hpo-miR-10160-3p, hpo-miR-10160-5p, hpo-miR-1175-3p, hpo-miR-1175-5p, hpo-miR-124-3p, hpo-miR-124-5p, hpo-miR-1822-3p, hpo-miR-1822-5p, hpo-miR-2-3p, hpo-miR-2-5p, hpo-miR-228-3p, hpo-miR-228-5p, hpo-miR-232a-3p, hpo-miR-232a-5p, hpo-miR-232b-3p, hpo-miR-232b-5p, hpo-miR-233-3p, hpo-miR-233-5p, hpo-miR-234-3p, hpo-miR-234-5p, hpo-miR-235-3p, hpo-miR-235-5p, hpo-miR-236-3p, hpo-miR-236-5p, hpo-miR-240-3p, hpo-miR-240-5p, hpo-miR-250-3p, hpo-miR-250-5p, hpo-miR-252-3p, hpo-miR-252-5p, hpo-miR-255-3p, hpo-miR-255-5p, hpo-miR-259-3p, hpo-miR-259-5p, hpo-miR-2709-3p, hpo-miR-2709-5p, hpo-miR-277-3p, hpo-miR-277-5p, hpo-miR-307-3p, hpo-miR-307-5p, hpo-miR-34-3p, hpo-miR-34-5p, hpo-miR-36a-3p, hpo-miR-36a-5p, hpo-miR-36b-3p, hpo-miR-36b-5p, hpo-miR-37-3p, hpo-miR-37-5p, hpo-miR-40a-3p, hpo-miR-40a-5p, hpo-miR-40b-3p, hpo-miR-40b-5p, hpo-miR-40c-3p, hpo-miR-40c-5p, hpo-miR-40d-3p, hpo-miR-40d-5p, hpo-miR-40e-3p, hpo-miR-40e-5p, hpo-miR-40f-3p, hpo-miR-40f-5p, hpo-miR-40g-3p, hpo-miR-40g-5p, hpo-miR-40 h-3p, hpo-miR-40 h-5p, hpo-miR-43a-3p, hpo-miR-43a-5p, hpo-miR-43b-3p, hpo-miR-43b-5p, hpo-miR-45-3p, hpo-miR-45-5p, hpo-miR-46-3p, hpo-miR-46-5p, hpo-miR-50-3p, hpo-miR-50-5p, hpo-miR-5352-3p, hpo-miR-5352-5p, hpo-miR-54a-3p, hpo-miR-54a-5p, hpo-miR-54b-3p, hpo-miR-54b-5p, hpo-miR-55-3p, hpo-miR-55-5p, hpo-miR-5884-3p, hpo-miR-5884-5p, hpo-miR-5885a-3p, hpo-miR-5885a-5p, hpo-miR-5885b-3p, hpo-miR-5885b-5p, hpo-miR-5885c-3p, hpo-miR-5885c-5p, hpo-miR-5895-3p, hpo-miR-5895-5p, hpo-miR-5896-3p, hpo-miR-5896-5p, hpo-miR-5897-3p, hpo-miR-5897-5p, hpo-miR-5898-3p, hpo-miR-5898-5p, hpo-miR-5899a-3p, hpo-miR-5899a-5p, hpo-miR-5899b-3p, hpo-miR-5899b-5p, hpo-miR-59-3p, hpo-miR-59-5p, hpo-miR-5902-3p, hpo-miR-5902-5p, hpo-miR-5906-3p, hpo-miR-5906-5p, hpo-miR-5908-3p, hpo-miR-5908-5p, hpo-miR-5915-3p, hpo-miR-5915-5p, hpo-miR-5932-3p, hpo-miR-5932-5p, hpo-miR-5948-3p, hpo-miR-5948-5p, hpo-miR-5976-3p, hpo-miR-5976-5p, hpo-miR-5983-3p, hpo-miR-5983-5p, hpo-miR-60a-3p, hpo-miR-60a-5p, hpo-miR-60b-3p, hpo-miR-60b-5p, hpo-miR-61a-3p, hpo-miR-61a-5p, hpo-miR-61b-3p, hpo-miR-61b-5p, hpo-miR-63a-3p, hpo-miR-63a-5p, hpo-miR-63b-3p, hpo-miR-63b-5p, hpo-miR-7-3p, hpo-miR-7-5p, hpo-miR-71-3p, hpo-miR-71-5p, hpo-miR-72-3p, hpo-miR-72-5p, hpo-miR-750-3p, hpo-miR-750-5p, hpo-miR-76-3p, hpo-miR-76-5p, hpo-miR-79-3p, hpo-miR-79-5p, hpo-miR-790-3p, hpo-miR-790-5p, hpo-miR-81-3p, hpo-miR-81-5p, hpo-miR-83-3p, hpo-miR-83-5p, hpo-miR-84a-3p, hpo-miR-84a-5p, hpo-miR-86-3p, hpo-miR-86-5p, hpo-miR-87a-3p, hpo-miR-87a-5p, hpo-miR-87b-3p, hpo-miR-87b-5p, hpo-miR-9-3p, hpo-miR-9-5p, hpo-miR-993-3p, hpo-miR-993-5p, hru-miR-1984, hru-miR-1985, hru-miR-1986, hru-miR-1990, hru-miR-1991, hsa-let-7a-2-3p, hsa-let-7a-3p, hsa-let-7a-5p, hsa-let-7b-3p, hsa-let-7b-5p, hsa-let-7c-3p, hsa-let-7c-5p, hsa-let-7d-3p, hsa-let-7d-5p, hsa-let-7e-3p, hsa-let-7e-5p, hsa-let-7f-1-3p, hsa-let-7f-2-3p, hsa-let-7f-5p, hsa-let-7g-3p, hsa-let-7g-5p, hsa-let-7i-3p, hsa-let-7i-5p, hsa-miR-1-3p, hsa-miR-1-5p, hsa-miR-100-3p, hsa-miR-100-5p, hsa-miR-101-2-5p, hsa-miR-101-3p, hsa-miR-101-5p, hsa-miR-10226, hsa-miR-10392-3p, hsa-miR-10392-5p, hsa-miR-10393-3p, hsa-miR-10393-5p, hsa-miR-10394-3p, hsa-miR-10394-5p, hsa-miR-10395-3p, hsa-miR-10395-5p, hsa-miR-10396a-3p, hsa-miR-10396a-5p, hsa-miR-10396b-3p, hsa-miR-10396b-5p, hsa-miR-10397-3p, hsa-miR-10397-5p, hsa-miR-10398-3p, hsa-miR-10398-5p, hsa-miR-10399-3p, hsa-miR-10399-5p, hsa-miR-103a-1-5p, hsa-miR-103a-2-5p, hsa-miR-103a-3p, hsa-miR-103b, hsa-miR-10400-3p, hsa-miR-10400-5p, hsa-miR-10401-3p, hsa-miR-10401-5p, hsa-miR-105-3p, hsa-miR-105-5p, hsa-miR-10522-5p, hsa-miR-10523-5p, hsa-miR-10524-5p, hsa-miR-10525-3p, hsa-miR-10526-3p, hsa-miR-10527-5p, hsa-miR-106a-3p, hsa-miR-106a-5p, hsa-miR-106b-3p, hsa-miR-106b-5p, hsa-miR-107, hsa-miR-10a-3p, hsa-miR-10a-5p, hsa-miR-10b-3p, hsa-miR-10b-5p, hsa-miR-11181-3p, hsa-miR-11181-5p, hsa-miR-11399, hsa-miR-11400, hsa-miR-11401, hsa-miR-1178-3p, hsa-miR-1178-5p, hsa-miR-1179, hsa-miR-1180-3p, hsa-miR-1180-5p, hsa-miR-1181, hsa-miR-1182, hsa-miR-1183, hsa-miR-1184, hsa-miR-1185-1-3p, hsa-miR-1185-2-3p, hsa-miR-1185-5p, hsa-miR-1193, hsa-miR-1197, hsa-miR-1199-3p, hsa-miR-1199-5p, hsa-miR-1200, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-12113, hsa-miR-12114, hsa-miR-12115, hsa-miR-12116, hsa-miR-12117, hsa-miR-12118, hsa-miR-12119, hsa-miR-12120, hsa-miR-12121, hsa-miR-12122, hsa-miR-12123, hsa-miR-12124, hsa-miR-12125, hsa-miR-12126, hsa-miR-12127, hsa-miR-12128, hsa-miR-12129, hsa-miR-12130, hsa-miR-12131, hsa-miR-12132, hsa-miR-12133, hsa-miR-12135, hsa-miR-12136, hsa-miR-122-3p, hsa-miR-122-5p, hsa-miR-1224-3p, hsa-miR-1224-5p, hsa-miR-1225-3p, hsa-miR-1225-5p, hsa-miR-1226-3p, hsa-miR-1226-5p, hsa-miR-1227-3p, hsa-miR-1227-5p, hsa-miR-1228-3p, hsa-miR-1228-5p, hsa-miR-1229-3p, hsa-miR-1229-5p, hsa-miR-122b-3p, hsa-miR-122b-5p, hsa-miR-1231, hsa-miR-1233-3p, hsa-miR-1233-5p, hsa-miR-1234-3p, hsa-miR-1236-3p, hsa-miR-1236-5p, hsa-miR-1237-3p, hsa-miR-1237-5p, hsa-miR-1238-3p, hsa-miR-1238-5p, hsa-miR-124-3p, hsa-miR-124-5p, hsa-miR-1243, hsa-miR-1244, hsa-miR-1245a, hsa-miR-1245b-3p, hsa-miR-1245b-5p, hsa-miR-1246, hsa-miR-1247-3p, hsa-miR-1247-5p, hsa-miR-1248, hsa-miR-1249-3p, hsa-miR-1249-5p, hsa-miR-1250-3p, hsa-miR-1250-5p, hsa-miR-1251-3p, hsa-miR-1251-5p, hsa-miR-1252-3p, hsa-miR-1252-5p, hsa-miR-1253, hsa-miR-1255a, hsa-miR-1255b-2-3p, hsa-miR-1255b-5p, hsa-miR-1256, hsa-miR-1257, hsa-miR-1258, hsa-miR-125a-3p, hsa-miR-125a-5p, hsa-miR-125b-1-3p, hsa-miR-125b-2-3p, hsa-miR-125b-5p, hsa-miR-126-3p, hsa-miR-126-5p, hsa-miR-1260a, hsa-miR-1260b, hsa-miR-1261, hsa-miR-1262, hsa-miR-1263, hsa-miR-1264, hsa-miR-1265, hsa-miR-1266-3p, hsa-miR-1266-5p, hsa-miR-1267, hsa-miR-1268a, hsa-miR-1268b, hsa-miR-1269a, hsa-miR-1269b, hsa-miR-127-3p, hsa-miR-127-5p, hsa-miR-1270, hsa-miR-1271-3p, hsa-miR-1271-5p, hsa-miR-1272, hsa-miR-1273c, hsa-miR-1273 h-3p, hsa-miR-1273 h-5p, hsa-miR-1275, hsa-miR-1276, hsa-miR-1277-3p, hsa-miR-1277-5p, hsa-miR-1278, hsa-miR-1279, hsa-miR-128-1-5p, hsa-miR-128-2-5p, hsa-miR-128-3p, hsa-miR-1281, hsa-miR-1282, hsa-miR-1283, hsa-miR-1284, hsa-miR-1285-3p, hsa-miR-1285-5p, hsa-miR-1286, hsa-miR-1287-3p, hsa-miR-1287-5p, hsa-miR-1288-3p, hsa-miR-1288-5p, hsa-miR-1289, hsa-miR-129-1-3p, hsa-miR-129-2-3p, hsa-miR-129-5p, hsa-miR-1290, hsa-miR-1291, hsa-miR-1292-3p, hsa-miR-1292-5p, hsa-miR-1293, hsa-miR-1294, hsa-miR-1295a, hsa-miR-1295b-3p, hsa-miR-1295b-5p, hsa-miR-1296-3p, hsa-miR-1296-5p, hsa-miR-1297, hsa-miR-1298-3p, hsa-miR-1298-5p, hsa-miR-1299, hsa-miR-1301-3p, hsa-miR-1301-5p, hsa-miR-1302, hsa-miR-1303, hsa-miR-1304-3p, hsa-miR-1304-5p, hsa-miR-1305, hsa-miR-1306-3p, hsa-miR-1306-5p, hsa-miR-1307-3p, hsa-miR-1307-5p, hsa-miR-130a-3p, hsa-miR-130a-5p, hsa-miR-130b-3p, hsa-miR-130b-5p, hsa-miR-132-3p, hsa-miR-132-5p, hsa-miR-1321, hsa-miR-1322, hsa-miR-1323, hsa-miR-1324, hsa-miR-133a-3p, hsa-miR-133a-5p, hsa-miR-133b, hsa-miR-134-3p, hsa-miR-134-5p, hsa-miR-1343-3p, hsa-miR-1343-5p, hsa-miR-135a-2-3p, hsa-miR-135a-3p, hsa-miR-135a-5p, hsa-miR-135b-3p, hsa-miR-135b-5p, hsa-miR-136-3p, hsa-miR-136-5p, hsa-miR-137-3p, hsa-miR-137-5p, hsa-miR-138-1-3p, hsa-miR-138-2-3p, hsa-miR-138-5p, hsa-miR-139-3p, hsa-miR-139-5p, hsa-miR-140-3p, hsa-miR-140-5p, hsa-miR-141-3p, hsa-miR-141-5p, hsa-miR-142-3p, hsa-miR-142-5p, hsa-miR-143-3p, hsa-miR-143-5p, hsa-miR-144-3p, hsa-miR-144-5p, hsa-miR-145-3p, hsa-miR-145-5p, hsa-miR-1468-3p, hsa-miR-1468-5p, hsa-miR-1469, hsa-miR-146a-3p, hsa-miR-146a-5p, hsa-miR-146b-3p, hsa-miR-146b-5p, hsa-miR-1470, hsa-miR-1471, hsa-miR-147a, hsa-miR-147b-3p, hsa-miR-147b-5p, hsa-miR-148a-3p, hsa-miR-148a-5p, hsa-miR-148b-3p, hsa-miR-148b-5p, hsa-miR-149-3p, hsa-miR-149-5p, hsa-miR-150-3p, hsa-miR-150-5p, hsa-miR-151a-3p, hsa-miR-151a-5p, hsa-miR-151b, hsa-miR-152-3p, hsa-miR-152-5p, hsa-miR-153-3p, hsa-miR-153-5p, hsa-miR-1537-3p, hsa-miR-1537-5p, hsa-miR-1538, hsa-miR-1539, hsa-miR-154-3p, hsa-miR-154-5p, hsa-miR-155-3p, hsa-miR-155-5p, hsa-miR-1587, hsa-miR-15a-3p, hsa-miR-15a-5p, hsa-miR-15b-3p, hsa-miR-15b-5p, hsa-miR-16-1-3p, hsa-miR-16-2-3p, hsa-miR-16-5p, hsa-miR-17-3p, hsa-miR-17-5p, hsa-miR-181a-2-3p, hsa-miR-181a-3p, hsa-miR-181a-5p, hsa-miR-181b-2-3p, hsa-miR-181b-3p, hsa-miR-181b-5p, hsa-miR-181c-3p, hsa-miR-181c-5p, hsa-miR-181d-3p, hsa-miR-181d-5p, hsa-miR-182-3p, hsa-miR-182-5p, hsa-miR-1825, hsa-miR-1827, hsa-miR-183-3p, hsa-miR-183-5p, hsa-miR-184, hsa-miR-1843, hsa-miR-185-3p, hsa-miR-185-5p, hsa-miR-186-3p, hsa-miR-186-5p, hsa-miR-187-3p, hsa-miR-187-5p, hsa-miR-188-3p, hsa-miR-188-5p, hsa-miR-18a-3p, hsa-miR-18a-5p, hsa-miR-18b-3p, hsa-miR-18b-5p, hsa-miR-1908-3p, hsa-miR-1908-5p, hsa-miR-1909-3p, hsa-miR-1909-5p, hsa-miR-190a-3p, hsa-miR-190a-5p, hsa-miR-190b-3p, hsa-miR-190b-5p, hsa-miR-191-3p, hsa-miR-191-5p, hsa-miR-1910-3p, hsa-miR-1910-5p, hsa-miR-1911-3p, hsa-miR-1911-5p, hsa-miR-1912-3p, hsa-miR-1912-5p, hsa-miR-1913, hsa-miR-1914-3p, hsa-miR-1914-5p, hsa-miR-1915-3p, hsa-miR-1915-5p, hsa-miR-192-3p, hsa-miR-192-5p, hsa-miR-193a-3p, hsa-miR-193a-5p, hsa-miR-193b-3p, hsa-miR-193b-5p, hsa-miR-194-3p, hsa-miR-194-5p, hsa-miR-195-3p, hsa-miR-195-5p, hsa-miR-196a-1-3p, hsa-miR-196a-3p, hsa-miR-196a-5p, hsa-miR-196b-3p, hsa-miR-196b-5p, hsa-miR-197-3p, hsa-miR-197-5p, hsa-miR-1972, hsa-miR-1973, hsa-miR-1976, hsa-miR-198, hsa-miR-199a-3p, hsa-miR-199a-5p, hsa-miR-199b-3p, hsa-miR-199b-5p, hsa-miR-19a-3p, hsa-miR-19a-5p, hsa-miR-19b-1-5p, hsa-miR-19b-2-5p, hsa-miR-19b-3p, hsa-miR-200a-3p, hsa-miR-200a-5p, hsa-miR-200b-3p, hsa-miR-200b-5p, hsa-miR-200c-3p, hsa-miR-200c-5p, hsa-miR-202-3p, hsa-miR-202-5p, hsa-miR-203a-3p, hsa-miR-203a-5p, hsa-miR-203b-3p, hsa-miR-203b-5p, hsa-miR-204-3p, hsa-miR-204-5p, hsa-miR-205-3p, hsa-miR-205-5p, hsa-miR-2052, hsa-miR-2053, hsa-miR-2054, hsa-miR-206, hsa-miR-208a-3p, hsa-miR-208a-5p, hsa-miR-208b-3p, hsa-miR-208b-5p, hsa-miR-20a-3p, hsa-miR-20a-5p, hsa-miR-20b-3p, hsa-miR-20b-5p, hsa-miR-21-3p, hsa-miR-21-5p, hsa-miR-210-3p, hsa-miR-210-5p, hsa-miR-211-3p, hsa-miR-211-5p, hsa-miR-2110, hsa-miR-2113, hsa-miR-2114-3p, hsa-miR-2114-5p, hsa-miR-2115-3p, hsa-miR-2115-5p, hsa-miR-2116-3p, hsa-miR-2116-5p, hsa-miR-2117, hsa-miR-212-3p, hsa-miR-212-5p, hsa-miR-214-3p, hsa-miR-214-5p, hsa-miR-215-3p, hsa-miR-215-5p, hsa-miR-216a-3p, hsa-miR-216a-5p, hsa-miR-216b-3p, hsa-miR-216b-5p, hsa-miR-217-3p, hsa-miR-217-5p, hsa-miR-218-1-3p, hsa-miR-218-2-3p, hsa-miR-218-5p, hsa-miR-219a-1-3p, hsa-miR-219a-2-3p, hsa-miR-219a-5p, hsa-miR-219b-3p, hsa-miR-219b-5p, hsa-miR-22-3p, hsa-miR-22-5p, hsa-miR-221-3p, hsa-miR-221-5p, hsa-miR-222-3p, hsa-miR-222-5p, hsa-miR-223-3p, hsa-miR-223-5p, hsa-miR-224-3p, hsa-miR-224-5p, hsa-miR-2276-3p, hsa-miR-2276-5p, hsa-miR-2277-3p, hsa-miR-2277-5p, hsa-miR-2278, hsa-miR-2355-3p, hsa-miR-2355-5p, hsa-miR-2392, hsa-miR-23a-3p, hsa-miR-23a-5p, hsa-miR-23b-3p, hsa-miR-23b-5p, hsa-miR-23c, hsa-miR-24-1-5p, hsa-miR-24-2-5p, hsa-miR-24-3p, hsa-miR-2467-3p, hsa-miR-2467-5p, hsa-miR-25-3p, hsa-miR-25-5p, hsa-miR-2681-3p, hsa-miR-2681-5p, hsa-miR-2682-3p, hsa-miR-2682-5p, hsa-miR-26a-1-3p, hsa-miR-26a-2-3p, hsa-miR-26a-5p, hsa-miR-26b-3p, hsa-miR-26b-5p, hsa-miR-27a-3p, hsa-miR-27a-5p, hsa-miR-27b-3p, hsa-miR-27b-5p, hsa-miR-28-3p, hsa-miR-28-5p, hsa-miR-2861, hsa-miR-2909, hsa-miR-296-3p, hsa-miR-296-5p, hsa-miR-297, hsa-miR-298, hsa-miR-299-3p, hsa-miR-299-5p, hsa-miR-29a-3p, hsa-miR-29a-5p, hsa-miR-29b-1-5p, hsa-miR-29b-2-5p, hsa-miR-29b-3p, hsa-miR-29c-3p, hsa-miR-29c-5p, hsa-miR-300, hsa-miR-301a-3p, hsa-miR-301a-5p, hsa-miR-301b-3p, hsa-miR-301b-5p, hsa-miR-302a-3p, hsa-miR-302a-5p, hsa-miR-302b-3p, hsa-miR-302b-5p, hsa-miR-302c-3p, hsa-miR-302c-5p, hsa-miR-302d-3p, hsa-miR-302d-5p, hsa-miR-302e, hsa-miR-302f, hsa-miR-3059-3p, hsa-miR-3059-5p, hsa-miR-3064-3p, hsa-miR-3064-5p, hsa-miR-3065-3p, hsa-miR-3065-5p, hsa-miR-3074-3p, hsa-miR-3074-5p, hsa-miR-3085-3p, hsa-miR-3085-5p, hsa-miR-30a-3p, hsa-miR-30a-5p, hsa-miR-30b-3p, hsa-miR-30b-5p, hsa-miR-30c-1-3p, hsa-miR-30c-2-3p, hsa-miR-30c-5p, hsa-miR-30d-3p, hsa-miR-30d-5p, hsa-miR-30e-3p, hsa-miR-30e-5p, hsa-miR-31-3p, hsa-miR-31-5p, hsa-miR-3115, hsa-miR-3116, hsa-miR-3117-3p, hsa-miR-3117-5p, hsa-miR-3118, hsa-miR-3119, hsa-miR-3120-3p, hsa-miR-3120-5p, hsa-miR-3121-3p, hsa-miR-3121-5p, hsa-miR-3122, hsa-miR-3123, hsa-miR-3124-3p, hsa-miR-3124-5p, hsa-miR-3125, hsa-miR-3126-3p, hsa-miR-3126-5p, hsa-miR-3127-3p, hsa-miR-3127-5p, hsa-miR-3128, hsa-miR-3129-3p, hsa-miR-3129-5p, hsa-miR-3130-3p, hsa-miR-3130-5p, hsa-miR-3131, hsa-miR-3132, hsa-miR-3133, hsa-miR-3134, hsa-miR-3135a, hsa-miR-3135b, hsa-miR-3136-3p, hsa-miR-3136-5p, hsa-miR-3137, hsa-miR-3138, hsa-miR-3139, hsa-miR-3140-3p, hsa-miR-3140-5p, hsa-miR-3141, hsa-miR-3142, hsa-miR-3143, hsa-miR-3144-3p, hsa-miR-3144-5p, hsa-miR-3145-3p, hsa-miR-3145-5p, hsa-miR-3146, hsa-miR-3147, hsa-miR-3148, hsa-miR-3149, hsa-miR-3150a-3p, hsa-miR-3150a-5p, hsa-miR-3150b-3p, hsa-miR-3150b-5p, hsa-miR-3151-3p, hsa-miR-3151-5p, hsa-miR-3152-3p, hsa-miR-3152-5p, hsa-miR-3153, hsa-miR-3154, hsa-miR-3155a, hsa-miR-3155b, hsa-miR-3156-3p, hsa-miR-3156-5p, hsa-miR-3157-3p, hsa-miR-3157-5p, hsa-miR-3158-3p, hsa-miR-3158-5p, hsa-miR-3159, hsa-miR-3160-3p, hsa-miR-3160-5p, hsa-miR-3161, hsa-miR-3162-3p, hsa-miR-3162-5p, hsa-miR-3163, hsa-miR-3164, hsa-miR-3165, hsa-miR-3166, hsa-miR-3167, hsa-miR-3168, hsa-miR-3169, hsa-miR-3170, hsa-miR-3171, hsa-miR-3173-3p, hsa-miR-3173-5p, hsa-miR-3174, hsa-miR-3175, hsa-miR-3176, hsa-miR-3177-3p, hsa-miR-3177-5p, hsa-miR-3178, hsa-miR-3179, hsa-miR-3180, hsa-miR-3180-3p, hsa-miR-3180-5p, hsa-miR-3181, hsa-miR-3182, hsa-miR-3183, hsa-miR-3184-3p, hsa-miR-3184-5p, hsa-miR-3185, hsa-miR-3186-3p, hsa-miR-3186-5p, hsa-miR-3187-3p, hsa-miR-3187-5p, hsa-miR-3188, hsa-miR-3189-3p, hsa-miR-3189-5p, hsa-miR-3190-3p, hsa-miR-3190-5p, hsa-miR-3191-3p, hsa-miR-3191-5p, hsa-miR-3192-3p, hsa-miR-3192-5p, hsa-miR-3193, hsa-miR-3194-3p, hsa-miR-3194-5p, hsa-miR-3195, hsa-miR-3196, hsa-miR-3197, hsa-miR-3198, hsa-miR-3199, hsa-miR-32-3p, hsa-miR-32-5p, hsa-miR-3200-3p, hsa-miR-3200-5p, hsa-miR-3201, hsa-miR-3202, hsa-miR-320a-3p, hsa-miR-320a-5p, hsa-miR-320b, hsa-miR-320c, hsa-miR-320d, hsa-miR-320e, hsa-miR-323a-3p, hsa-miR-323a-5p, hsa-miR-323b-3p, hsa-miR-323b-5p, hsa-miR-324-3p, hsa-miR-324-5p, hsa-miR-325, hsa-miR-326, hsa-miR-328-3p, hsa-miR-328-5p, hsa-miR-329-3p, hsa-miR-329-5p, hsa-miR-330-3p, hsa-miR-330-5p, hsa-miR-331-3p, hsa-miR-331-5p, hsa-miR-335-3p, hsa-miR-335-5p, hsa-miR-337-3p, hsa-miR-337-5p, hsa-miR-338-3p, hsa-miR-338-5p, hsa-miR-339-3p, hsa-miR-339-5p, hsa-miR-33a-3p, hsa-miR-33a-5p, hsa-miR-33b-3p, hsa-miR-33b-5p, hsa-miR-340-3p, hsa-miR-340-5p, hsa-miR-342-3p, hsa-miR-342-5p, hsa-miR-345-3p, hsa-miR-345-5p, hsa-miR-346, hsa-miR-34a-3p, hsa-miR-34a-5p, hsa-miR-34b-3p, hsa-miR-34b-5p, hsa-miR-34c-3p, hsa-miR-34c-5p, hsa-miR-3529-3p, hsa-miR-3529-5p, hsa-miR-3605-3p, hsa-miR-3605-5p, hsa-miR-3606-3p, hsa-miR-3606-5p, hsa-miR-3609, hsa-miR-361-3p, hsa-miR-361-5p, hsa-miR-3610, hsa-miR-3611, hsa-miR-3612, hsa-miR-3613-3p, hsa-miR-3613-5p, hsa-miR-3614-3p, hsa-miR-3614-5p, hsa-miR-3615, hsa-miR-3616-3p, hsa-miR-3616-5p, hsa-miR-3617-3p, hsa-miR-3617-5p, hsa-miR-3618, hsa-miR-3619-3p, hsa-miR-3619-5p, hsa-miR-362-3p, hsa-miR-362-5p, hsa-miR-3620-3p, hsa-miR-3620-5p, hsa-miR-3621, hsa-miR-3622a-3p, hsa-miR-3622a-5p, hsa-miR-3622b-3p, hsa-miR-3622b-5p, hsa-miR-363-3p, hsa-miR-363-5p, hsa-miR-3646, hsa-miR-3648, hsa-miR-3649, hsa-miR-3650, hsa-miR-3651, hsa-miR-3652, hsa-miR-3654, hsa-miR-3655, hsa-miR-3657, hsa-miR-3658, hsa-miR-3659, hsa-miR-365a-3p, hsa-miR-365a-5p, hsa-miR-365b-3p, hsa-miR-365b-5p, hsa-miR-3660, hsa-miR-3661, hsa-miR-3662, hsa-miR-3663-3p, hsa-miR-3663-5p, hsa-miR-3664-3p, hsa-miR-3664-5p, hsa-miR-3665, hsa-miR-3666, hsa-miR-3667-3p, hsa-miR-3667-5p, hsa-miR-3668, hsa-miR-367-3p, hsa-miR-367-5p, hsa-miR-3670, hsa-miR-3671, hsa-miR-3672, hsa-miR-3674, hsa-miR-3675-3p, hsa-miR-3675-5p, hsa-miR-3677-3p, hsa-miR-3677-5p, hsa-miR-3678-3p, hsa-miR-3678-5p, hsa-miR-3679-3p, hsa-miR-3679-5p, hsa-miR-3680-3p, hsa-miR-3680-5p, hsa-miR-3681-3p, hsa-miR-3681-5p, hsa-miR-3682-3p, hsa-miR-3682-5p, hsa-miR-3683, hsa-miR-3684, hsa-miR-3685, hsa-miR-3686, hsa-miR-3688-3p, hsa-miR-3688-5p, hsa-miR-3689a-3p, hsa-miR-3689a-5p, hsa-miR-3689b-3p, hsa-miR-3689b-5p, hsa-miR-3689c, hsa-miR-3689d, hsa-miR-3689e, hsa-miR-3689f, hsa-miR-369-3p, hsa-miR-369-5p, hsa-miR-3690, hsa-miR-3691-3p, hsa-miR-3691-5p, hsa-miR-3692-3p, hsa-miR-3692-5p, hsa-miR-370-3p, hsa-miR-370-5p, hsa-miR-3713, hsa-miR-3714, hsa-miR-371a-3p, hsa-miR-371a-5p, hsa-miR-371b-3p, hsa-miR-371b-5p, hsa-miR-372-3p, hsa-miR-372-5p, hsa-miR-373-3p, hsa-miR-373-5p, hsa-miR-374a-3p, hsa-miR-374a-5p, hsa-miR-374b-3p, hsa-miR-374b-5p, hsa-miR-374c-3p, hsa-miR-374c-5p, hsa-miR-375-3p, hsa-miR-375-5p, hsa-miR-376a-2-5p, hsa-miR-376a-3p, hsa-miR-376a-5p, hsa-miR-376b-3p, hsa-miR-376b-5p, hsa-miR-376c-3p, hsa-miR-376c-5p, hsa-miR-377-3p, hsa-miR-377-5p, hsa-miR-378a-3p, hsa-miR-378a-5p, hsa-miR-378b, hsa-miR-378c, hsa-miR-378d, hsa-miR-378e, hsa-miR-378f, hsa-miR-378g, hsa-miR-378h, hsa-miR-378i, hsa-miR-378j, hsa-miR-379-3p, hsa-miR-379-5p, hsa-miR-380-3p, hsa-miR-380-5p, hsa-miR-381-3p, hsa-miR-381-5p, hsa-miR-382-3p, hsa-miR-382-5p, hsa-miR-383-3p, hsa-miR-383-5p, hsa-miR-384, hsa-miR-3907, hsa-miR-3908, hsa-miR-3909, hsa-miR-3910, hsa-miR-3911, hsa-miR-3912-3p, hsa-miR-3912-5p, hsa-miR-3913-3p, hsa-miR-3913-5p, hsa-miR-3914, hsa-miR-3915, hsa-miR-3916, hsa-miR-3917, hsa-miR-3918, hsa-miR-3919, hsa-miR-3920, hsa-miR-3921, hsa-miR-3922-3p, hsa-miR-3922-5p, hsa-miR-3923, hsa-miR-3924, hsa-miR-3925-3p, hsa-miR-3925-5p, hsa-miR-3926, hsa-miR-3927-3p, hsa-miR-3927-5p, hsa-miR-3928-3p, hsa-miR-3928-5p, hsa-miR-3929, hsa-miR-3934-3p, hsa-miR-3934-5p, hsa-miR-3935, hsa-miR-3936, hsa-miR-3937, hsa-miR-3938, hsa-miR-3939, hsa-miR-3940-3p, hsa-miR-3940-5p, hsa-miR-3941, hsa-miR-3942-3p, hsa-miR-3942-5p, hsa-miR-3943, hsa-miR-3944-3p, hsa-miR-3944-5p, hsa-miR-3945, hsa-miR-3960, hsa-miR-3972, hsa-miR-3973, hsa-miR-3974, hsa-miR-3975, hsa-miR-3976, hsa-miR-3977, hsa-miR-3978, hsa-miR-409-3p, hsa-miR-409-5p, hsa-miR-410-3p, hsa-miR-410-5p, hsa-miR-411-3p, hsa-miR-411-5p, hsa-miR-412-3p, hsa-miR-412-5p, hsa-miR-421, hsa-miR-422a, hsa-miR-423-3p, hsa-miR-423-5p, hsa-miR-424-3p, hsa-miR-424-5p, hsa-miR-425-3p, hsa-miR-425-5p, hsa-miR-4251, hsa-miR-4252, hsa-miR-4253, hsa-miR-4254, hsa-miR-4255, hsa-miR-4256, hsa-miR-4257, hsa-miR-4258, hsa-miR-4259, hsa-miR-4260, hsa-miR-4261, hsa-miR-4262, hsa-miR-4263, hsa-miR-4264, hsa-miR-4265, hsa-miR-4266, hsa-miR-4267, hsa-miR-4268, hsa-miR-4269, hsa-miR-4270, hsa-miR-4271, hsa-miR-4272, hsa-miR-4273, hsa-miR-4274, hsa-miR-4275, hsa-miR-4276, hsa-miR-4277, hsa-miR-4278, hsa-miR-4279, hsa-miR-4280, hsa-miR-4281, hsa-miR-4282, hsa-miR-4283, hsa-miR-4284, hsa-miR-4285, hsa-miR-4286, hsa-miR-4287, hsa-miR-4288, hsa-miR-4289, hsa-miR-429, hsa-miR-4290, hsa-miR-4291, hsa-miR-4292, hsa-miR-4293, hsa-miR-4294, hsa-miR-4295, hsa-miR-4296, hsa-miR-4297, hsa-miR-4298, hsa-miR-4299, hsa-miR-4300, hsa-miR-4301, hsa-miR-4302, hsa-miR-4303, hsa-miR-4304, hsa-miR-4305, hsa-miR-4306, hsa-miR-4307, hsa-miR-4308, hsa-miR-4309, hsa-miR-431-3p, hsa-miR-431-5p, hsa-miR-4310, hsa-miR-4311, hsa-miR-4312, hsa-miR-4313, hsa-miR-4314, hsa-miR-4315, hsa-miR-4316, hsa-miR-4317, hsa-miR-4318, hsa-miR-4319, hsa-miR-432-3p, hsa-miR-432-5p, hsa-miR-4320, hsa-miR-4321, hsa-miR-4322, hsa-miR-4323, hsa-miR-4324, hsa-miR-4325, hsa-miR-4326, hsa-miR-4327, hsa-miR-4328, hsa-miR-4329, hsa-miR-433-3p, hsa-miR-433-5p, hsa-miR-4330, hsa-miR-4418, hsa-miR-4420, hsa-miR-4421, hsa-miR-4422, hsa-miR-4423-3p, hsa-miR-4423-5p, hsa-miR-4424, hsa-miR-4425, hsa-miR-4426, hsa-miR-4427, hsa-miR-4428, hsa-miR-4429, hsa-miR-4430, hsa-miR-4431, hsa-miR-4432, hsa-miR-4433a-3p, hsa-miR-4433a-5p, hsa-miR-4433b-3p, hsa-miR-4433b-5p, hsa-miR-4434, hsa-miR-4435, hsa-miR-4436a, hsa-miR-4436b-3p, hsa-miR-4436b-5p, hsa-miR-4437, hsa-miR-4438, hsa-miR-4439, hsa-miR-4440, hsa-miR-4441, hsa-miR-4442, hsa-miR-4443, hsa-miR-4444, hsa-miR-4445-3p, hsa-miR-4445-5p, hsa-miR-4446-3p, hsa-miR-4446-5p, hsa-miR-4447, hsa-miR-4448, hsa-miR-4449, hsa-miR-4450, hsa-miR-4451, hsa-miR-4452, hsa-miR-4453, hsa-miR-4454, hsa-miR-4455, hsa-miR-4456, hsa-miR-4457, hsa-miR-4458, hsa-miR-4460, hsa-miR-4462, hsa-miR-4463, hsa-miR-4464, hsa-miR-4465, hsa-miR-4466, hsa-miR-4467, hsa-miR-4468, hsa-miR-4469, hsa-miR-4470, hsa-miR-4471, hsa-miR-4472, hsa-miR-4473, hsa-miR-4474-3p, hsa-miR-4474-5p, hsa-miR-4475, hsa-miR-4476, hsa-miR-4477a, hsa-miR-4477b, hsa-miR-4478, hsa-miR-4479, hsa-miR-448, hsa-miR-4480, hsa-miR-4481, hsa-miR-4482-3p, hsa-miR-4482-5p, hsa-miR-4483, hsa-miR-4484, hsa-miR-4485-3p, hsa-miR-4485-5p, hsa-miR-4486, hsa-miR-4487, hsa-miR-4488, hsa-miR-4489, hsa-miR-4490, hsa-miR-4491, hsa-miR-4492, hsa-miR-4493, hsa-miR-4494, hsa-miR-4495, hsa-miR-4496, hsa-miR-4497, hsa-miR-4498, hsa-miR-4499, hsa-miR-449a, hsa-miR-449b-3p, hsa-miR-449b-5p, hsa-miR-449c-3p, hsa-miR-449c-5p, hsa-miR-4500, hsa-miR-4501, hsa-miR-4502, hsa-miR-4503, hsa-miR-4504, hsa-miR-4505, hsa-miR-4506, hsa-miR-4507, hsa-miR-4508, hsa-miR-4509, hsa-miR-450a-1-3p, hsa-miR-450a-2-3p, hsa-miR-450a-5p, hsa-miR-450b-3p, hsa-miR-450b-5p, hsa-miR-4510, hsa-miR-4511, hsa-miR-4512, hsa-miR-4513, hsa-miR-4514, hsa-miR-4515, hsa-miR-4516, hsa-miR-4517, hsa-miR-4518, hsa-miR-4519, hsa-miR-451a, hsa-miR-451b, hsa-miR-452-3p, hsa-miR-452-5p, hsa-miR-4520-2-3p, hsa-miR-4520-3p, hsa-miR-4520-5p, hsa-miR-4521, hsa-miR-4522, hsa-miR-4523, hsa-miR-4524a-3p, hsa-miR-4524a-5p, hsa-miR-4524b-3p, hsa-miR-4524b-5p, hsa-miR-4525, hsa-miR-4526, hsa-miR-4527, hsa-miR-4528, hsa-miR-4529-3p, hsa-miR-4529-5p, hsa-miR-4530, hsa-miR-4531, hsa-miR-4533, hsa-miR-4534, hsa-miR-4535, hsa-miR-4536-3p, hsa-miR-4536-5p, hsa-miR-4537, hsa-miR-4538, hsa-miR-4539, hsa-miR-454-3p, hsa-miR-454-5p, hsa-miR-4540, hsa-miR-455-3p, hsa-miR-455-5p, hsa-miR-4632-3p, hsa-miR-4632-5p, hsa-miR-4633-3p, hsa-miR-4633-5p, hsa-miR-4634, hsa-miR-4635, hsa-miR-4636, hsa-miR-4637, hsa-miR-4638-3p, hsa-miR-4638-5p, hsa-miR-4639-3p, hsa-miR-4639-5p, hsa-miR-4640-3p, hsa-miR-4640-5p, hsa-miR-4641, hsa-miR-4642, hsa-miR-4643, hsa-miR-4644, hsa-miR-4645-3p, hsa-miR-4645-5p, hsa-miR-4646-3p, hsa-miR-4646-5p, hsa-miR-4647, hsa-miR-4648, hsa-miR-4649-3p, hsa-miR-4649-5p, hsa-miR-4650-3p, hsa-miR-4650-5p, hsa-miR-4651, hsa-miR-4652-3p, hsa-miR-4652-5p, hsa-miR-4653-3p, hsa-miR-4653-5p, hsa-miR-4654, hsa-miR-4655-3p, hsa-miR-4655-5p, hsa-miR-4656, hsa-miR-4657, hsa-miR-4658, hsa-miR-4659a-3p, hsa-miR-4659a-5p, hsa-miR-4659b-3p, hsa-miR-4659b-5p, hsa-miR-466, hsa-miR-4660, hsa-miR-4661-3p, hsa-miR-4661-5p, hsa-miR-4662a-3p, hsa-miR-4662a-5p, hsa-miR-4662b, hsa-miR-4663, hsa-miR-4664-3p, hsa-miR-4664-5p, hsa-miR-4665-3p, hsa-miR-4665-5p, hsa-miR-4666a-3p, hsa-miR-4666a-5p, hsa-miR-4666b, hsa-miR-4667-3p, hsa-miR-4667-5p, hsa-miR-4668-3p, hsa-miR-4668-5p, hsa-miR-4669, hsa-miR-4670-3p, hsa-miR-4670-5p, hsa-miR-4671-3p, hsa-miR-4671-5p, hsa-miR-4672, hsa-miR-4673, hsa-miR-4674, hsa-miR-4675, hsa-miR-4676-3p, hsa-miR-4676-5p, hsa-miR-4677-3p, hsa-miR-4677-5p, hsa-miR-4678, hsa-miR-4679, hsa-miR-4680-3p, hsa-miR-4680-5p, hsa-miR-4681, hsa-miR-4682, hsa-miR-4683, hsa-miR-4684-3p, hsa-miR-4684-5p, hsa-miR-4685-3p, hsa-miR-4685-5p, hsa-miR-4686, hsa-miR-4687-3p, hsa-miR-4687-5p, hsa-miR-4688, hsa-miR-4689, hsa-miR-4690-3p, hsa-miR-4690-5p, hsa-miR-4691-3p, hsa-miR-4691-5p, hsa-miR-4692, hsa-miR-4693-3p, hsa-miR-4693-5p, hsa-miR-4694-3p, hsa-miR-4694-5p, hsa-miR-4695-3p, hsa-miR-4695-5p, hsa-miR-4696, hsa-miR-4697-3p, hsa-miR-4697-5p, hsa-miR-4698, hsa-miR-4699-3p, hsa-miR-4699-5p, hsa-miR-4700-3p, hsa-miR-4700-5p, hsa-miR-4701-3p, hsa-miR-4701-5p, hsa-miR-4703-3p, hsa-miR-4703-5p, hsa-miR-4704-3p, hsa-miR-4704-5p, hsa-miR-4705, hsa-miR-4706, hsa-miR-4707-3p, hsa-miR-4707-5p, hsa-miR-4708-3p, hsa-miR-4708-5p, hsa-miR-4709-3p, hsa-miR-4709-5p, hsa-miR-4710, hsa-miR-4711-3p, hsa-miR-4711-5p, hsa-miR-4712-3p, hsa-miR-4712-5p, hsa-miR-4713-3p, hsa-miR-4713-5p, hsa-miR-4714-3p, hsa-miR-4714-5p, hsa-miR-4715-3p, hsa-miR-4715-5p, hsa-miR-4716-3p, hsa-miR-4716-5p, hsa-miR-4717-3p, hsa-miR-4717-5p, hsa-miR-4718, hsa-miR-4719, hsa-miR-4720-3p, hsa-miR-4720-5p, hsa-miR-4721, hsa-miR-4722-3p, hsa-miR-4722-5p, hsa-miR-4723-3p, hsa-miR-4723-5p, hsa-miR-4724-3p, hsa-miR-4724-5p, hsa-miR-4725-3p, hsa-miR-4725-5p, hsa-miR-4726-3p, hsa-miR-4726-5p, hsa-miR-4727-3p, hsa-miR-4727-5p, hsa-miR-4728-3p, hsa-miR-4728-5p, hsa-miR-4729, hsa-miR-4730, hsa-miR-4731-3p, hsa-miR-4731-5p, hsa-miR-4732-3p, hsa-miR-4732-5p, hsa-miR-4733-3p, hsa-miR-4733-5p, hsa-miR-4734, hsa-miR-4735-3p, hsa-miR-4735-5p, hsa-miR-4736, hsa-miR-4737, hsa-miR-4738-3p, hsa-miR-4738-5p, hsa-miR-4739, hsa-miR-4740-3p, hsa-miR-4740-5p, hsa-miR-4741, hsa-miR-4742-3p, hsa-miR-4742-5p, hsa-miR-4743-3p, hsa-miR-4743-5p, hsa-miR-4744, hsa-miR-4745-3p, hsa-miR-4745-5p, hsa-miR-4746-3p, hsa-miR-4746-5p, hsa-miR-4747-3p, hsa-miR-4747-5p, hsa-miR-4748, hsa-miR-4749-3p, hsa-miR-4749-5p, hsa-miR-4750-3p, hsa-miR-4750-5p, hsa-miR-4751, hsa-miR-4752, hsa-miR-4753-3p, hsa-miR-4753-5p, hsa-miR-4754, hsa-miR-4755-3p, hsa-miR-4755-5p, hsa-miR-4756-3p, hsa-miR-4756-5p, hsa-miR-4757-3p, hsa-miR-4757-5p, hsa-miR-4758-3p, hsa-miR-4758-5p, hsa-miR-4759, hsa-miR-4760-3p, hsa-miR-4760-5p, hsa-miR-4761-3p, hsa-miR-4761-5p, hsa-miR-4762-3p, hsa-miR-4762-5p, hsa-miR-4763-3p, hsa-miR-4763-5p, hsa-miR-4764-3p, hsa-miR-4764-5p, hsa-miR-4765, hsa-miR-4766-3p, hsa-miR-4766-5p, hsa-miR-4767, hsa-miR-4768-3p, hsa-miR-4768-5p, hsa-miR-4769-3p, hsa-miR-4769-5p, hsa-miR-4770, hsa-miR-4771, hsa-miR-4772-3p, hsa-miR-4772-5p, hsa-miR-4773, hsa-miR-4774-3p, hsa-miR-4774-5p, hsa-miR-4775, hsa-miR-4776-3p, hsa-miR-4776-5p, hsa-miR-4777-3p, hsa-miR-4777-5p, hsa-miR-4778-3p, hsa-miR-4778-5p, hsa-miR-4779, hsa-miR-4780, hsa-miR-4781-3p, hsa-miR-4781-5p, hsa-miR-4782-3p, hsa-miR-4782-5p, hsa-miR-4783-3p, hsa-miR-4783-5p, hsa-miR-4784, hsa-miR-4785, hsa-miR-4786-3p, hsa-miR-4786-5p, hsa-miR-4787-3p, hsa-miR-4787-5p, hsa-miR-4788, hsa-miR-4789-3p, hsa-miR-4789-5p, hsa-miR-4790-3p, hsa-miR-4790-5p, hsa-miR-4791, hsa-miR-4793-3p, hsa-miR-4793-5p, hsa-miR-4794, hsa-miR-4795-3p, hsa-miR-4795-5p, hsa-miR-4796-3p, hsa-miR-4796-5p, hsa-miR-4797-3p, hsa-miR-4797-5p, hsa-miR-4798-3p, hsa-miR-4798-5p, hsa-miR-4799-3p, hsa-miR-4799-5p, hsa-miR-4800-3p, hsa-miR-4800-5p, hsa-miR-4801, hsa-miR-4802-3p, hsa-miR-4802-5p, hsa-miR-4803, hsa-miR-4804-3p, hsa-miR-4804-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a-3p, hsa-miR-487a-5p, hsa-miR-487b-3p, hsa-miR-487b-5p, hsa-miR-488-3p, hsa-miR-488-5p, hsa-miR-489-3p, hsa-miR-489-5p, hsa-miR-490-3p, hsa-miR-490-5p, hsa-miR-491-3p, hsa-miR-491-5p, hsa-miR-492, hsa-miR-493-3p, hsa-miR-493-5p, hsa-miR-494-3p, hsa-miR-494-5p, hsa-miR-495-3p, hsa-miR-495-5p, hsa-miR-496, hsa-miR-497-3p, hsa-miR-497-5p, hsa-miR-498-3p, hsa-miR-498-5p, hsa-miR-4999-3p, hsa-miR-4999-5p, hsa-miR-499a-3p, hsa-miR-499a-5p, hsa-miR-499b-3p, hsa-miR-499b-5p, hsa-miR-5000-3p, hsa-miR-5000-5p, hsa-miR-5001-3p, hsa-miR-5001-5p, hsa-miR-5002-3p, hsa-miR-5002-5p, hsa-miR-5003-3p, hsa-miR-5003-5p, hsa-miR-5004-3p, hsa-miR-5004-5p, hsa-miR-5006-3p, hsa-miR-5006-5p, hsa-miR-5007-3p, hsa-miR-5007-5p, hsa-miR-5008-3p, hsa-miR-5008-5p, hsa-miR-5009-3p, hsa-miR-5009-5p, hsa-miR-500a-3p, hsa-miR-500a-5p, hsa-miR-500b-3p, hsa-miR-500b-5p, hsa-miR-501-3p, hsa-miR-501-5p, hsa-miR-5010-3p, hsa-miR-5010-5p, hsa-miR-5011-3p, hsa-miR-5011-5p, hsa-miR-502-3p, hsa-miR-502-5p, hsa-miR-503-3p, hsa-miR-503-5p, hsa-miR-504-3p, hsa-miR-504-5p, hsa-miR-5047, hsa-miR-505-3p, hsa-miR-505-5p, hsa-miR-506-3p, hsa-miR-506-5p, hsa-miR-507, hsa-miR-508-3p, hsa-miR-508-5p, hsa-miR-5087, hsa-miR-5088-3p, hsa-miR-5088-5p, hsa-miR-5089-3p, hsa-miR-5089-5p, hsa-miR-509-3-5p, hsa-miR-509-3p, hsa-miR-509-5p, hsa-miR-5090, hsa-miR-5091, hsa-miR-5092, hsa-miR-5093, hsa-miR-5094, hsa-miR-510-3p, hsa-miR-510-5p, hsa-miR-5100, hsa-miR-511-3p, hsa-miR-511-5p, hsa-miR-512-3p, hsa-miR-512-5p, hsa-miR-513a-3p, hsa-miR-513a-5p, hsa-miR-513b-3p, hsa-miR-513b-5p, hsa-miR-513c-3p, hsa-miR-513c-5p, hsa-miR-514a-3p, hsa-miR-514a-5p, hsa-miR-514b-3p, hsa-miR-514b-5p, hsa-miR-515-3p, hsa-miR-515-5p, hsa-miR-516a-3p, hsa-miR-516a-5p, hsa-miR-516b-3p, hsa-miR-516b-5p, hsa-miR-517-5p, hsa-miR-517a-3p, hsa-miR-517b-3p, hsa-miR-517c-3p, hsa-miR-5186, hsa-miR-5187-3p, hsa-miR-5187-5p, hsa-miR-5188, hsa-miR-5189-3p, hsa-miR-5189-5p, hsa-miR-518a-3p, hsa-miR-518a-5p, hsa-miR-518b, hsa-miR-518c-3p, hsa-miR-518c-5p, hsa-miR-518d-3p, hsa-miR-518d-5p, hsa-miR-518e-3p, hsa-miR-518e-5p, hsa-miR-518f-3p, hsa-miR-518f-5p, hsa-miR-5190, hsa-miR-5191, hsa-miR-5192, hsa-miR-5193, hsa-miR-5194, hsa-miR-5195-3p, hsa-miR-5195-5p, hsa-miR-5196-3p, hsa-miR-5196-5p, hsa-miR-5197-3p, hsa-miR-5197-5p, hsa-miR-519a-2-5p, hsa-miR-519a-3p, hsa-miR-519a-5p, hsa-miR-519b-3p, hsa-miR-519b-5p, hsa-miR-519c-3p, hsa-miR-519c-5p, hsa-miR-519d-3p, hsa-miR-519d-5p, hsa-miR-519e-3p, hsa-miR-519e-5p, hsa-miR-520a-3p, hsa-miR-520a-5p, hsa-miR-520b-3p, hsa-miR-520b-5p, hsa-miR-520c-3p, hsa-miR-520c-5p, hsa-miR-520d-3p, hsa-miR-520d-5p, hsa-miR-520e-3p, hsa-miR-520e-5p, hsa-miR-520f-3p, hsa-miR-520f-5p, hsa-miR-520g-3p, hsa-miR-520g-5p, hsa-miR-520h, hsa-miR-521, hsa-miR-522-3p, hsa-miR-522-5p, hsa-miR-523-3p, hsa-miR-523-5p, hsa-miR-524-3p, hsa-miR-524-5p, hsa-miR-525-3p, hsa-miR-525-5p, hsa-miR-526a-3p, hsa-miR-526a-5p, hsa-miR-526b-3p, hsa-miR-526b-5p, hsa-miR-527, hsa-miR-532-3p, hsa-miR-532-5p, hsa-miR-539-3p, hsa-miR-539-5p, hsa-miR-541-3p, hsa-miR-541-5p, hsa-miR-542-3p, hsa-miR-542-5p, hsa-miR-543, hsa-miR-544a, hsa-miR-544b, hsa-miR-545-3p, hsa-miR-545-5p, hsa-miR-548a-3p, hsa-miR-548a-5p, hsa-miR-548aa, hsa-miR-548ab, hsa-miR-548ac, hsa-miR-548ad-3p, hsa-miR-548ad-5p, hsa-miR-548ae-3p, hsa-miR-548ae-5p, hsa-miR-548ag, hsa-miR-548ah-3p, hsa-miR-548ah-5p, hsa-miR-548ai, hsa-miR-548aj-3p, hsa-miR-548aj-5p, hsa-miR-548ak, hsa-miR-548al, hsa-miR-548 am-3p, hsa-miR-548 am-5p, hsa-miR-548an, hsa-miR-548ao-3p, hsa-miR-548ao-5p, hsa-miR-548ap-3p, hsa-miR-548ap-5p, hsa-miR-548aq-3p, hsa-miR-548aq-5p, hsa-miR-548ar-3p, hsa-miR-548ar-5p, hsa-miR-548as-3p, hsa-miR-548as-5p, hsa-miR-548at-3p, hsa-miR-548at-5p, hsa-miR-548au-3p, hsa-miR-548au-5p, hsa-miR-548av-3p, hsa-miR-548av-5p, hsa-miR-548aw, hsa-miR-548ax, hsa-miR-548ay-3p, hsa-miR-548ay-5p, hsa-miR-548az-3p, hsa-miR-548az-5p, hsa-miR-548b-3p, hsa-miR-548b-5p, hsa-miR-548ba, hsa-miR-548bb-3p, hsa-miR-548bb-5p, hsa-miR-548bc, hsa-miR-548c-3p, hsa-miR-548c-5p, hsa-miR-548d-3p, hsa-miR-548d-5p, hsa-miR-548e-3p, hsa-miR-548e-5p, hsa-miR-548f-3p, hsa-miR-548f-5p, hsa-miR-548g-3p, hsa-miR-548g-5p, hsa-miR-548h-3p, hsa-miR-548 h-5p, hsa-miR-548i, hsa-miR-548j-3p, hsa-miR-548j-5p, hsa-miR-548k, hsa-miR-5481, hsa-miR-548m, hsa-miR-548n, hsa-miR-5480-3p, hsa-miR-5480-5p, hsa-miR-548p, hsa-miR-548q, hsa-miR-548s, hsa-miR-548t-3p, hsa-miR-548t-5p, hsa-miR-548u, hsa-miR-548v, hsa-miR-548w, hsa-miR-548x-3p, hsa-miR-548x-5p, hsa-miR-548y, hsa-miR-548z, hsa-miR-549a-3p, hsa-miR-549a-5p, hsa-miR-550a-3-5p, hsa-miR-550a-3p, hsa-miR-550a-5p, hsa-miR-550b-2-5p, hsa-miR-550b-3p, hsa-miR-551a, hsa-miR-551b-3p, hsa-miR-551b-5p, hsa-miR-552-3p, hsa-miR-552-5p, hsa-miR-553, hsa-miR-554, hsa-miR-555, hsa-miR-556-3p, hsa-miR-556-5p, hsa-miR-557, hsa-miR-5571-3p, hsa-miR-5571-5p, hsa-miR-5572, hsa-miR-5579-3p, hsa-miR-5579-5p, hsa-miR-558, hsa-miR-5580-3p, hsa-miR-5580-5p, hsa-miR-5581-3p, hsa-miR-5581-5p, hsa-miR-5582-3p, hsa-miR-5582-5p, hsa-miR-5583-3p, hsa-miR-5583-5p, hsa-miR-5584-3p, hsa-miR-5584-5p, hsa-miR-5585-3p, hsa-miR-5585-5p, hsa-miR-5586-3p, hsa-miR-5586-5p, hsa-miR-5587-3p, hsa-miR-5587-5p, hsa-miR-5588-3p, hsa-miR-5588-5p, hsa-miR-5589-3p, hsa-miR-5589-5p, hsa-miR-559, hsa-miR-5590-3p, hsa-miR-5590-5p, hsa-miR-5591-3p, hsa-miR-5591-5p, hsa-miR-561-3p, hsa-miR-561-5p, hsa-miR-562, hsa-miR-563, hsa-miR-564, hsa-miR-567, hsa-miR-568, hsa-miR-5680, hsa-miR-5681a, hsa-miR-5681b, hsa-miR-5682, hsa-miR-5683, hsa-miR-5684, hsa-miR-5685, hsa-miR-5687, hsa-miR-5688, hsa-miR-5689, hsa-miR-569, hsa-miR-5690, hsa-miR-5691, hsa-miR-5692a, hsa-miR-5692b, hsa-miR-5692c, hsa-miR-5693, hsa-miR-5694, hsa-miR-5695, hsa-miR-5696, hsa-miR-5697, hsa-miR-5698, hsa-miR-5699-3p, hsa-miR-5699-5p, hsa-miR-570-3p, hsa-miR-570-5p, hsa-miR-5700, hsa-miR-5701, hsa-miR-5702, hsa-miR-5703, hsa-miR-5704, hsa-miR-5705, hsa-miR-5706, hsa-miR-5707, hsa-miR-5708, hsa-miR-571, hsa-miR-572, hsa-miR-573, hsa-miR-5739, hsa-miR-574-3p, hsa-miR-574-5p, hsa-miR-575, hsa-miR-576-3p, hsa-miR-576-5p, hsa-miR-577, hsa-miR-578, hsa-miR-5787, hsa-miR-579-3p, hsa-miR-579-5p, hsa-miR-580-3p, hsa-miR-580-5p, hsa-miR-581, hsa-miR-582-3p, hsa-miR-582-5p, hsa-miR-583, hsa-miR-584-3p, hsa-miR-584-5p, hsa-miR-585-3p, hsa-miR-585-5p, hsa-miR-586, hsa-miR-587, hsa-miR-588, hsa-miR-589-3p, hsa-miR-589-5p, hsa-miR-590-3p, hsa-miR-590-5p, hsa-miR-591, hsa-miR-592, hsa-miR-593-3p, hsa-miR-593-5p, hsa-miR-595, hsa-miR-596, hsa-miR-597-3p, hsa-miR-597-5p, hsa-miR-598-3p, hsa-miR-598-5p, hsa-miR-599, hsa-miR-600, hsa-miR-601, hsa-miR-602, hsa-miR-603, hsa-miR-604, hsa-miR-605-3p, hsa-miR-605-5p, hsa-miR-606, hsa-miR-6068, hsa-miR-6069, hsa-miR-607, hsa-miR-6070, hsa-miR-6071, hsa-miR-6072, hsa-miR-6073, hsa-miR-6074, hsa-miR-6075, hsa-miR-6076, hsa-miR-6077, hsa-miR-6078, hsa-miR-6079, hsa-miR-608, hsa-miR-6080, hsa-miR-6081, hsa-miR-6082, hsa-miR-6083, hsa-miR-6084, hsa-miR-6085, hsa-miR-6086, hsa-miR-6088, hsa-miR-6089, hsa-miR-609, hsa-miR-6090, hsa-miR-610, hsa-miR-611, hsa-miR-612, hsa-miR-6124, hsa-miR-6125, hsa-miR-6126, hsa-miR-6127, hsa-miR-6128, hsa-miR-6129, hsa-miR-613, hsa-miR-6130, hsa-miR-6131, hsa-miR-6132, hsa-miR-6133, hsa-miR-6134, hsa-miR-614, hsa-miR-615-3p, hsa-miR-615-5p, hsa-miR-616-3p, hsa-miR-616-5p, hsa-miR-6165, hsa-miR-617, hsa-miR-618, hsa-miR-619-3p, hsa-miR-619-5p, hsa-miR-620, hsa-miR-621, hsa-miR-622, hsa-miR-623, hsa-miR-624-3p, hsa-miR-624-5p, hsa-miR-625-3p, hsa-miR-625-5p, hsa-miR-626, hsa-miR-627-3p, hsa-miR-627-5p, hsa-miR-628-3p, hsa-miR-628-5p, hsa-miR-629-3p, hsa-miR-629-5p, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642a-3p, hsa-miR-642a-5p, hsa-miR-642b-3p, hsa-miR-642b-5p, hsa-miR-643, hsa-miR-644a, hsa-miR-645, hsa-miR-646, hsa-miR-647, hsa-miR-648, hsa-miR-649, hsa-miR-6499-3p, hsa-miR-6499-5p, hsa-miR-650, hsa-miR-6500-3p, hsa-miR-6500-5p, hsa-miR-6501-3p, hsa-miR-6501-5p, hsa-miR-6502-3p, hsa-miR-6502-5p, hsa-miR-6503-3p, hsa-miR-6503-5p, hsa-miR-6504-3p, hsa-miR-6504-5p, hsa-miR-6505-3p, hsa-miR-6505-5p, hsa-miR-6506-3p, hsa-miR-6506-5p, hsa-miR-6507-3p, hsa-miR-6507-5p, hsa-miR-6508-3p, hsa-miR-6508-5p, hsa-miR-6509-3p, hsa-miR-6509-5p, hsa-miR-651-3p, hsa-miR-651-5p, hsa-miR-6510-3p, hsa-miR-6510-5p, hsa-miR-6511a-3p, hsa-miR-6511a-5p, hsa-miR-6511b-3p, hsa-miR-6511b-5p, hsa-miR-6512-3p, hsa-miR-6512-5p, hsa-miR-6513-3p, hsa-miR-6513-5p, hsa-miR-6514-3p, hsa-miR-6514-5p, hsa-miR-6515-3p, hsa-miR-6515-5p, hsa-miR-6516-3p, hsa-miR-6516-5p, hsa-miR-652-3p, hsa-miR-652-5p, hsa-miR-6529-3p, hsa-miR-6529-5p, hsa-miR-653-3p, hsa-miR-653-5p, hsa-miR-654-3p, hsa-miR-654-5p, hsa-miR-655-3p, hsa-miR-655-5p, hsa-miR-656-3p, hsa-miR-656-5p, hsa-miR-657, hsa-miR-658, hsa-miR-659-3p, hsa-miR-659-5p, hsa-miR-660-3p, hsa-miR-660-5p, hsa-miR-661, hsa-miR-662, hsa-miR-663a, hsa-miR-663b, hsa-miR-664a-3p, hsa-miR-664a-5p, hsa-miR-664b-3p, hsa-miR-664b-5p, hsa-miR-665, hsa-miR-668-3p, hsa-miR-668-5p, hsa-miR-670-3p, hsa-miR-670-5p, hsa-miR-671-3p, hsa-miR-671-5p, hsa-miR-6715a-3p, hsa-miR-6715b-3p, hsa-miR-6715b-5p, hsa-miR-6716-3p, hsa-miR-6716-5p, hsa-miR-6717-5p, hsa-miR-6718-5p, hsa-miR-6719-3p, hsa-miR-6720-3p, hsa-miR-6720-5p, hsa-miR-6721-5p, hsa-miR-6722-3p, hsa-miR-6722-5p, hsa-miR-6724-5p, hsa-miR-6726-3p, hsa-miR-6726-5p, hsa-miR-6727-3p, hsa-miR-6727-5p, hsa-miR-6728-3p, hsa-miR-6728-5p, hsa-miR-6729-3p, hsa-miR-6729-5p, hsa-miR-6730-3p, hsa-miR-6730-5p, hsa-miR-6731-3p, hsa-miR-6731-5p, hsa-miR-6732-3p, hsa-miR-6732-5p, hsa-miR-6733-3p, hsa-miR-6733-5p, hsa-miR-6734-3p, hsa-miR-6734-5p, hsa-miR-6735-3p, hsa-miR-6735-5p, hsa-miR-6736-3p, hsa-miR-6736-5p, hsa-miR-6737-3p, hsa-miR-6737-5p, hsa-miR-6738-3p, hsa-miR-6738-5p, hsa-miR-6739-3p, hsa-miR-6739-5p, hsa-miR-6740-3p, hsa-miR-6740-5p, hsa-miR-6741-3p, hsa-miR-6741-5p, hsa-miR-6742-3p, hsa-miR-6742-5p, hsa-miR-6743-3p, hsa-miR-6743-5p, hsa-miR-6744-3p, hsa-miR-6744-5p, hsa-miR-6745, hsa-miR-6746-3p, hsa-miR-6746-5p, hsa-miR-6747-3p, hsa-miR-6747-5p, hsa-miR-6748-3p, hsa-miR-6748-5p, hsa-miR-6749-3p, hsa-miR-6749-5p, hsa-miR-675-3p, hsa-miR-675-5p, hsa-miR-6750-3p, hsa-miR-6750-5p, hsa-miR-6751-3p, hsa-miR-6751-5p, hsa-miR-6752-3p, hsa-miR-6752-5p, hsa-miR-6753-3p, hsa-miR-6753-5p, hsa-miR-6754-3p, hsa-miR-6754-5p, hsa-miR-6755-3p, hsa-miR-6755-5p, hsa-miR-6756-3p, hsa-miR-6756-5p, hsa-miR-6757-3p, hsa-miR-6757-5p, hsa-miR-6758-3p, hsa-miR-6758-5p, hsa-miR-6759-3p, hsa-miR-6759-5p, hsa-miR-676-3p, hsa-miR-676-5p, hsa-miR-6760-3p, hsa-miR-6760-5p, hsa-miR-6761-3p, hsa-miR-6761-5p, hsa-miR-6762-3p, hsa-miR-6762-5p, hsa-miR-6763-3p, hsa-miR-6763-5p, hsa-miR-6764-3p, hsa-miR-6764-5p, hsa-miR-6765-3p, hsa-miR-6765-5p, hsa-miR-6766-3p, hsa-miR-6766-5p, hsa-miR-6767-3p, hsa-miR-6767-5p, hsa-miR-6768-3p, hsa-miR-6768-5p, hsa-miR-6769a-3p, hsa-miR-6769a-5p, hsa-miR-6769b-3p, hsa-miR-6769b-5p, hsa-miR-6770-3p, hsa-miR-6770-5p, hsa-miR-6771-3p, hsa-miR-6771-5p, hsa-miR-6772-3p, hsa-miR-6772-5p, hsa-miR-6773-3p, hsa-miR-6773-5p, hsa-miR-6774-3p, hsa-miR-6774-5p, hsa-miR-6775-3p, hsa-miR-6775-5p, hsa-miR-6776-3p, hsa-miR-6776-5p, hsa-miR-6777-3p, hsa-miR-6777-5p, hsa-miR-6778-3p, hsa-miR-6778-5p, hsa-miR-6779-3p, hsa-miR-6779-5p, hsa-miR-6780a-3p, hsa-miR-6780a-5p, hsa-miR-6780b-3p, hsa-miR-6780b-5p, hsa-miR-6781-3p, hsa-miR-6781-5p, hsa-miR-6782-3p, hsa-miR-6782-5p, hsa-miR-6783-3p, hsa-miR-6783-5p, hsa-miR-6784-3p, hsa-miR-6784-5p, hsa-miR-6785-3p, hsa-miR-6785-5p, hsa-miR-6786-3p, hsa-miR-6786-5p, hsa-miR-6787-3p, hsa-miR-6787-5p, hsa-miR-6788-3p, hsa-miR-6788-5p, hsa-miR-6789-3p, hsa-miR-6789-5p, hsa-miR-6790-3p, hsa-miR-6790-5p, hsa-miR-6791-3p, hsa-miR-6791-5p, hsa-miR-6792-3p, hsa-miR-6792-5p, hsa-miR-6793-3p, hsa-miR-6793-5p, hsa-miR-6794-3p, hsa-miR-6794-5p, hsa-miR-6795-3p, hsa-miR-6795-5p, hsa-miR-6796-3p, hsa-miR-6796-5p, hsa-miR-6797-3p, hsa-miR-6797-5p, hsa-miR-6798-3p, hsa-miR-6798-5p, hsa-miR-6799-3p, hsa-miR-6799-5p, hsa-miR-6800-3p, hsa-miR-6800-5p, hsa-miR-6801-3p, hsa-miR-6801-5p, hsa-miR-6802-3p, hsa-miR-6802-5p, hsa-miR-6803-3p, hsa-miR-6803-5p, hsa-miR-6804-3p, hsa-miR-6804-5p, hsa-miR-6805-3p, hsa-miR-6805-5p, hsa-miR-6806-3p, hsa-miR-6806-5p, hsa-miR-6807-3p, hsa-miR-6807-5p, hsa-miR-6808-3p, hsa-miR-6808-5p, hsa-miR-6809-3p, hsa-miR-6809-5p, hsa-miR-6810-3p, hsa-miR-6810-5p, hsa-miR-6811-3p, hsa-miR-6811-5p, hsa-miR-6812-3p, hsa-miR-6812-5p, hsa-miR-6813-3p, hsa-miR-6813-5p, hsa-miR-6814-3p, hsa-miR-6814-5p, hsa-miR-6815-3p, hsa-miR-6815-5p, hsa-miR-6816-3p, hsa-miR-6816-5p, hsa-miR-6817-3p, hsa-miR-6817-5p, hsa-miR-6818-3p, hsa-miR-6818-5p, hsa-miR-6819-3p, hsa-miR-6819-5p, hsa-miR-6820-3p, hsa-miR-6820-5p, hsa-miR-6821-3p, hsa-miR-6821-5p, hsa-miR-6822-3p, hsa-miR-6822-5p, hsa-miR-6823-3p, hsa-miR-6823-5p, hsa-miR-6824-3p, hsa-miR-6824-5p, hsa-miR-6825-3p, hsa-miR-6825-5p, hsa-miR-6826-3p, hsa-miR-6826-5p, hsa-miR-6827-3p, hsa-miR-6827-5p, hsa-miR-6828-3p, hsa-miR-6828-5p, hsa-miR-6829-3p, hsa-miR-6829-5p, hsa-miR-6830-3p, hsa-miR-6830-5p, hsa-miR-6831-3p, hsa-miR-6831-5p, hsa-miR-6832-3p, hsa-miR-6832-5p, hsa-miR-6833-3p, hsa-miR-6833-5p, hsa-miR-6834-3p, hsa-miR-6834-5p, hsa-miR-6835-3p, hsa-miR-6835-5p, hsa-miR-6836-3p, hsa-miR-6836-5p, hsa-miR-6837-3p, hsa-miR-6837-5p, hsa-miR-6838-3p, hsa-miR-6838-5p, hsa-miR-6839-3p, hsa-miR-6839-5p, hsa-miR-6840-3p, hsa-miR-6840-5p, hsa-miR-6841-3p, hsa-miR-6841-5p, hsa-miR-6842-3p, hsa-miR-6842-5p, hsa-miR-6843-3p, hsa-miR-6844, hsa-miR-6845-3p, hsa-miR-6845-5p, hsa-miR-6846-3p, hsa-miR-6846-5p, hsa-miR-6847-3p, hsa-miR-6847-5p, hsa-miR-6848-3p, hsa-miR-6848-5p, hsa-miR-6849-3p, hsa-miR-6849-5p, hsa-miR-6850-3p, hsa-miR-6850-5p, hsa-miR-6851-3p, hsa-miR-6851-5p, hsa-miR-6852-3p, hsa-miR-6852-5p, hsa-miR-6853-3p, hsa-miR-6853-5p, hsa-miR-6854-3p, hsa-miR-6854-5p, hsa-miR-6855-3p, hsa-miR-6855-5p, hsa-miR-6856-3p, hsa-miR-6856-5p, hsa-miR-6857-3p, hsa-miR-6857-5p, hsa-miR-6858-3p, hsa-miR-6858-5p, hsa-miR-6859-3p, hsa-miR-6859-5p, hsa-miR-6860, hsa-miR-6861-3p, hsa-miR-6861-5p, hsa-miR-6862-3p, hsa-miR-6862-5p, hsa-miR-6863, hsa-miR-6864-3p, hsa-miR-6864-5p, hsa-miR-6865-3p, hsa-miR-6865-5p, hsa-miR-6866-3p, hsa-miR-6866-5p, hsa-miR-6867-3p, hsa-miR-6867-5p, hsa-miR-6868-3p, hsa-miR-6868-5p, hsa-miR-6869-3p, hsa-miR-6869-5p, hsa-miR-6870-3p, hsa-miR-6870-5p, hsa-miR-6871-3p, hsa-miR-6871-5p, hsa-miR-6872-3p, hsa-miR-6872-5p, hsa-miR-6873-3p, hsa-miR-6873-5p, hsa-miR-6874-3p, hsa-miR-6874-5p, hsa-miR-6875-3p, hsa-miR-6875-5p, hsa-miR-6876-3p, hsa-miR-6876-5p, hsa-miR-6877-3p, hsa-miR-6877-5p, hsa-miR-6878-3p, hsa-miR-6878-5p, hsa-miR-6879-3p, hsa-miR-6879-5p, hsa-miR-6880-3p, hsa-miR-6880-5p, hsa-miR-6881-3p, hsa-miR-6881-5p, hsa-miR-6882-3p, hsa-miR-6882-5p, hsa-miR-6883-3p, hsa-miR-6883-5p, hsa-miR-6884-3p, hsa-miR-6884-5p, hsa-miR-6885-3p, hsa-miR-6885-5p, hsa-miR-6886-3p, hsa-miR-6886-5p, hsa-miR-6887-3p, hsa-miR-6887-5p, hsa-miR-6888-3p, hsa-miR-6888-5p, hsa-miR-6889-3p, hsa-miR-6889-5p, hsa-miR-6890-3p, hsa-miR-6890-5p, hsa-miR-6891-3p, hsa-miR-6891-5p, hsa-miR-6892-3p, hsa-miR-6892-5p, hsa-miR-6893-3p, hsa-miR-6893-5p, hsa-miR-6894-3p, hsa-miR-6894-5p, hsa-miR-6895-3p, hsa-miR-6895-5p, hsa-miR-7-1-3p, hsa-miR-7-2-3p, hsa-miR-7-5p, hsa-miR-708-3p, hsa-miR-708-5p, hsa-miR-7106-3p, hsa-miR-7106-5p, hsa-miR-7107-3p, hsa-miR-7107-5p, hsa-miR-7108-3p, hsa-miR-7108-5p, hsa-miR-7109-3p, hsa-miR-7109-5p, hsa-miR-711, hsa-miR-7110-3p, hsa-miR-7110-5p, hsa-miR-7111-3p, hsa-miR-7111-5p, hsa-miR-7112-3p, hsa-miR-7112-5p, hsa-miR-7113-3p, hsa-miR-7113-5p, hsa-miR-7114-3p, hsa-miR-7114-5p, hsa-miR-7150, hsa-miR-7151-3p, hsa-miR-7151-5p, hsa-miR-7152-3p, hsa-miR-7152-5p, hsa-miR-7153-3p, hsa-miR-7153-5p, hsa-miR-7154-3p, hsa-miR-7154-5p, hsa-miR-7155-3p, hsa-miR-7155-5p, hsa-miR-7156-3p, hsa-miR-7156-5p, hsa-miR-7157-3p, hsa-miR-7157-5p, hsa-miR-7158-3p, hsa-miR-7158-5p, hsa-miR-7159-3p, hsa-miR-7159-5p, hsa-miR-7160-3p, hsa-miR-7160-5p, hsa-miR-7161-3p, hsa-miR-7161-5p, hsa-miR-7162-3p, hsa-miR-7162-5p, hsa-miR-718, hsa-miR-744-3p, hsa-miR-744-5p, hsa-miR-7515, hsa-miR-758-3p, hsa-miR-758-5p, hsa-miR-759, hsa-miR-760, hsa-miR-761, hsa-miR-762, hsa-miR-764, hsa-miR-765, hsa-miR-766-3p, hsa-miR-766-5p, hsa-miR-767-3p, hsa-miR-767-5p, hsa-miR-769-3p, hsa-miR-769-5p, hsa-miR-770-5p, hsa-miR-7702, hsa-miR-7703, hsa-miR-7704, hsa-miR-7705, hsa-miR-7706, hsa-miR-7843-3p, hsa-miR-7843-5p, hsa-miR-7844-5p, hsa-miR-7845-5p, hsa-miR-7846-3p, hsa-miR-7847-3p, hsa-miR-7848-3p, hsa-miR-7849-3p, hsa-miR-7850-5p, hsa-miR-7851-3p, hsa-miR-7852-3p, hsa-miR-7853-5p, hsa-miR-7854-3p, hsa-miR-7855-5p, hsa-miR-7856-5p, hsa-miR-7973, hsa-miR-7974, hsa-miR-7975, hsa-miR-7976, hsa-miR-7977, hsa-miR-7978, hsa-miR-802, hsa-miR-8052, hsa-miR-8053, hsa-miR-8054, hsa-miR-8055, hsa-miR-8056, hsa-miR-8057, hsa-miR-8058, hsa-miR-8059, hsa-miR-8060, hsa-miR-8061, hsa-miR-8062, hsa-miR-8063, hsa-miR-8064, hsa-miR-8065, hsa-miR-8066, hsa-miR-8067, hsa-miR-8068, hsa-miR-8069, hsa-miR-8070, hsa-miR-8071, hsa-miR-8072, hsa-miR-8073, hsa-miR-8074, hsa-miR-8075, hsa-miR-8076, hsa-miR-8077, hsa-miR-8078, hsa-miR-8079, hsa-miR-8080, hsa-miR-8081, hsa-miR-8082, hsa-miR-8083, hsa-miR-8084, hsa-miR-8085, hsa-miR-8086, hsa-miR-8087, hsa-miR-8088, hsa-miR-8089, hsa-miR-8485, hsa-miR-873-3p, hsa-miR-873-5p, hsa-miR-874-3p, hsa-miR-874-5p, hsa-miR-875-3p, hsa-miR-875-5p, hsa-miR-876-3p, hsa-miR-876-5p, hsa-miR-877-3p, hsa-miR-877-5p, hsa-miR-885-3p, hsa-miR-885-5p, hsa-miR-887-3p, hsa-miR-887-5p, hsa-miR-888-3p, hsa-miR-888-5p, hsa-miR-889-3p, hsa-miR-889-5p, hsa-miR-890, hsa-miR-891a-3p, hsa-miR-891a-5p, hsa-miR-891b, hsa-miR-892a, hsa-miR-892b, hsa-miR-892c-3p, hsa-miR-892c-5p, hsa-miR-9-3p, hsa-miR-9-5p, hsa-miR-920, hsa-miR-921, hsa-miR-922, hsa-miR-924, hsa-miR-92a-1-5p, hsa-miR-92a-2-5p, hsa-miR-92a-3p, hsa-miR-92b-3p, hsa-miR-92b-5p, hsa-miR-93-3p, hsa-miR-93-5p, hsa-miR-933, hsa-miR-934, hsa-miR-935, hsa-miR-936, hsa-miR-937-3p, hsa-miR-937-5p, hsa-miR-938, hsa-miR-939-3p, hsa-miR-939-5p, hsa-miR-940, hsa-miR-941, hsa-miR-942-3p, hsa-miR-942-5p, hsa-miR-943, hsa-miR-944, hsa-miR-95-3p, hsa-miR-95-5p, hsa-miR-9500, hsa-miR-96-3p, hsa-miR-96-5p, hsa-miR-9718, hsa-miR-98-3p, hsa-miR-98-5p, hsa-miR-9851-3p, hsa-miR-9851-5p, hsa-miR-9898, hsa-miR-9899, hsa-miR-9900, hsa-miR-9901, hsa-miR-9902, hsa-miR-9903, hsa-miR-9983-3p, hsa-miR-9985, hsa-miR-9986, hsa-miR-99a-3p, hsa-miR-99a-5p, hsa-miR-99b-3p, hsa-miR-99b-5p, hsv1-miR-H1-3p, hsv1-miR-H1-5p, hsv1-miR-H11, hsv1-miR-H12, hsv1-miR-H13, hsv1-miR-H14-3p, hsv1-miR-H14-5p, hsv1-miR-H15, hsv1-miR-H16, hsv1-miR-H17, hsv1-miR-H18, hsv1-miR-H2-3p, hsv1-miR-H2-5p, hsv1-miR-H26, hsv1-miR-H27, hsv1-miR-H3-3p, hsv1-miR-H3-5p, hsv1-miR-H4-3p, hsv1-miR-H4-5p, hsv1-miR-H5-3p, hsv1-miR-H5-5p, hsv1-miR-H6-3p, hsv1-miR-H6-5p, hsv1-miR-H7-3p, hsv1-miR-H7-5p, hsv1-miR-H8-3p, hsv1-miR-H8-5p, hsv2-miR-H10, hsv2-miR-H11-3p, hsv2-miR-H11-5p, hsv2-miR-H12, hsv2-miR-H13, hsv2-miR-H19, hsv2-miR-H2, hsv2-miR-H20, hsv2-miR-H21, hsv2-miR-H22, hsv2-miR-H23-3p, hsv2-miR-H23-5p, hsv2-miR-H24, hsv2-miR-H25, hsv2-miR-H3, hsv2-miR-H4-3p, hsv2-miR-H4-5p, hsv2-miR-H5, hsv2-miR-H6-3p, hsv2-miR-H6-5p, hsv2-miR-H7-3p, hsv2-miR-H7-5p, hsv2-miR-H9-3p, hsv2-miR-H9-5p, htu-miR156a, htu-miR159a, htu-miR160a, htu-miR162a, htu-miR171a, htu-miR171b, htu-miR171c, htu-miR393a, htu-miR393b, htu-miR393c, htu-miR403a, htu-miR403b, htu-miR403c, htu-miR403d, htu-miR403e, htu-miR530, hvsa-miR-HSUR2-3p, hvsa-miR-HSUR2-5p, hvsa-miR-HSUR4-3p, hvsa-miR-HSUR4-5p, hvsa-miR-HSUR5-3p, hvsa-miR-HSUR5-5p, hvt-miR-H1, hvt-miR-H10, hvt-miR-H11, hvt-miR-H12-3p, hvt-miR-H12-5p, hvt-miR-H13, hvt-miR-H14-3p, hvt-miR-H14-5p, hvt-miR-H15-3p, hvt-miR-H15-5p, hvt-miR-H16-3p, hvt-miR-H16-5p, hvt-miR-H17-3p, hvt-miR-H17-5p, hvt-miR-H18-3p, hvt-miR-H18-5p, hvt-miR-H2, hvt-miR-H3-3p, hvt-miR-H3-5p, hvt-miR-H4-3p, hvt-miR-H4-5p, hvt-miR-H5-3p, hvt-miR-H5-5p, hvt-miR-H7-3p, hvt-miR-H7-5p, hvt-miR-H8, hvt-miR-H9-3p, hvt-miR-H9-5p, hvu-miR1120, hvu-miR1130, hvu-miR1436, hvu-miR156a, hvu-miR156b, hvu-miR159a, hvu-miR159b, hvu-miR166a, hvu-miR166b, hvu-miR166c, hvu-miR168-3p, hvu-miR168-5p, hvu-miR169, hvu-miR171-3p, hvu-miR171-5p, hvu-miR397a, hvu-miR397b-3p, hvu-miR399, hvu-miR444a, hvu-miR444b, hvu-miR5048a, hvu-miR5048b, hvu-miR5049a, hvu-miR5049b, hvu-miR5049c, hvu-miR5049d, hvu-miR5049e, hvu-miR5049f, hvu-miR5050, hvu-miR5051, hvu-miR5052, hvu-miR5053, hvu-miR6176, hvu-miR6177, hvu-miR6178, hvu-miR6179, hvu-miR6180, hvu-miR6181, hvu-miR6182, hvu-miR6183, hvu-miR6184, hvu-miR6185, hvu-miR6186, hvu-miR6187, hvu-miR6188, hvu-miR6189, hvu-miR6190, hvu-miR6191, hvu-miR6192, hvu-miR6193, hvu-miR6194, hvu-miR6195, hvu-miR6196, hvu-miR6197, hvu-miR6198, hvu-miR6199, hvu-miR6200, hvu-miR6201, hvu-miR6202, hvu-miR6203, hvu-miR6204, hvu-miR6205, hvu-miR6206, hvu-miR6207, hvu-miR6208, hvu-miR6209, hvu-miR6210, hvu-miR6211, hvu-miR6212, hvu-miR6213, hvu-miR6214, iltv-miR-11-3p, iltv-miR-11-5p, iltv-miR-12, iltv-miR-13, iltv-miR-14, iltv-miR-15-3p, iltv-miR-15-5p, iltv-miR-16-3p, iltv-miR-16-5p, iltv-miR-17, ipu-let-7a, ipu-let-7b, ipu-let-7c, ipu-let-7d, ipu-let-7e, ipu-let-7f, ipu-let-7g, ipu-let-7h, ipu-let-7i, ipu-let-7j, ipu-miR-1, ipu-miR-100, ipu-miR-101a, ipu-miR-101b, ipu-miR-103, ipu-miR-107a, ipu-miR-107b, ipu-miR-10a, ipu-miR-10b, ipu-miR-10c, ipu-miR-10d, ipu-miR-122, ipu-miR-124a, ipu-miR-125a, ipu-miR-125b, ipu-miR-125c, ipu-miR-126a, ipu-miR-126b, ipu-miR-128, ipu-miR-129, ipu-miR-129b, ipu-miR-130c, ipu-miR-132a, ipu-miR-132b, ipu-miR-133a, ipu-miR-133b, ipu-miR-133c, ipu-miR-135a, ipu-miR-135b, ipu-miR-135c, ipu-miR-137, ipu-miR-138, ipu-miR-1388, ipu-miR-139, ipu-miR-140, ipu-miR-141, ipu-miR-142, ipu-miR-143, ipu-miR-144, ipu-miR-145, ipu-miR-146a, ipu-miR-146b, ipu-miR-148, ipu-miR-150, ipu-miR-152, ipu-miR-153b, ipu-miR-155, ipu-miR-15a, ipu-miR-15b, ipu-miR-16b, ipu-miR-16c, ipu-miR-1788, ipu-miR-17a, ipu-miR-17b, ipu-miR-181a, ipu-miR-181b, ipu-miR-181c, ipu-miR-182, ipu-miR-183, ipu-miR-184, ipu-miR-187, ipu-miR-18a, ipu-miR-190a, ipu-miR-190b, ipu-miR-192, ipu-miR-194a, ipu-miR-196a, ipu-miR-196d, ipu-miR-199a-3p, ipu-miR-199a-5p, ipu-miR-199b, ipu-miR-19a, ipu-miR-19b, ipu-miR-200a, ipu-miR-200b, ipu-miR-200c, ipu-miR-202, ipu-miR-203a, ipu-miR-203b, ipu-miR-203c, ipu-miR-204, ipu-miR-205, ipu-miR-206, ipu-miR-20a, ipu-miR-21, ipu-miR-210, ipu-miR-212, ipu-miR-214, ipu-miR-216a, ipu-miR-216b, ipu-miR-217, ipu-miR-2187, ipu-miR-2188, ipu-miR-218a, ipu-miR-218b, ipu-miR-219a, ipu-miR-219b, ipu-miR-221, ipu-miR-222a, ipu-miR-223, ipu-miR-22a, ipu-miR-22b, ipu-miR-23a, ipu-miR-23b, ipu-miR-24, ipu-miR-24b, ipu-miR-25, ipu-miR-26a, ipu-miR-26b, ipu-miR-27a, ipu-miR-27b, ipu-miR-27c, ipu-miR-27d, ipu-miR-27e, ipu-miR-29a, ipu-miR-29b, ipu-miR-29c, ipu-miR-301a, ipu-miR-301c, ipu-miR-30a, ipu-miR-30b, ipu-miR-30c, ipu-miR-30d, ipu-miR-30e, ipu-miR-31, ipu-miR-338, ipu-miR-34a, ipu-miR-34b, ipu-miR-34c, ipu-miR-3618, ipu-miR-363, ipu-miR-365, ipu-miR-375, ipu-miR-429a, ipu-miR-429b, ipu-miR-454b, ipu-miR-455a, ipu-miR-455b, ipu-miR-456, ipu-miR-457a, ipu-miR-457b, ipu-miR-458, ipu-miR-459, ipu-miR-460, ipu-miR-462, ipu-miR-489, ipu-miR-499, ipu-miR-551, ipu-miR-7147, ipu-miR-724, ipu-miR-728, ipu-miR-730, ipu-miR-737, ipu-miR-7547, ipu-miR-7548, ipu-miR-7549, ipu-miR-7550, ipu-miR-7551, ipu-miR-7552, ipu-miR-7553, ipu-miR-7554, ipu-miR-7555, ipu-miR-7556, ipu-miR-7557, ipu-miR-7558a, ipu-miR-7558b, ipu-miR-7559, ipu-miR-7560, ipu-miR-7561, ipu-miR-7562, ipu-miR-7563a, ipu-miR-7563b, ipu-miR-7563c, ipu-miR-7564, ipu-miR-7565, ipu-miR-7566, ipu-miR-7567, ipu-miR-7568, ipu-miR-7569, ipu-miR-7570, ipu-miR-7571, ipu-miR-7572, ipu-miR-7573, ipu-miR-7574, ipu-miR-7575, ipu-miR-7576, ipu-miR-7577, ipu-miR-7a, ipu-miR-7b, ipu-miR-9, ipu-miR-92a, ipu-miR-92b, ipu-miR-96, ipu-miR-99a, ipu-miR-99b, isc-bantam, isc-let-7, isc-miR-1, isc-miR-10, isc-miR-100, isc-miR-12, isc-miR-124, isc-miR-133, isc-miR-137, isc-miR-153, isc-miR-184, isc-miR-1993, isc-miR-2001, isc-miR-219, isc-miR-252b, isc-miR-263a, isc-miR-275, isc-miR-276, isc-miR-278, isc-miR-279, isc-miR-285, isc-miR-2a, isc-miR-2b, isc-miR-305, isc-miR-307, isc-miR-315, isc-miR-317, isc-miR-375, isc-miR-3931, isc-miR-5305, isc-miR-5306, isc-miR-5307, isc-miR-5308, isc-miR-5309, isc-miR-5310, isc-miR-5311, isc-miR-5312, isc-miR-5313, isc-miR-5314, isc-miR-5315, isc-miR-7, isc-miR-71, isc-miR-750, isc-miR-79, isc-miR-8, isc-miR-87, isc-miR-96, isc-miR-993, isc-miR-iab-4, jcv-miR-J1-3p, jcv-miR-J1-5p, kshv-miR-K12-1-3p, kshv-miR-K12-1-5p, kshv-miR-K12-10a-3p, kshv-miR-K12-10a-5p, kshv-miR-K12-10b, kshv-miR-K12-11-3p, kshv-miR-K12-11-5p, kshv-miR-K12-12-3p, kshv-miR-K12-12-5p, kshv-miR-K12-2-3p, kshv-miR-K12-2-5p, kshv-miR-K12-3-3p, kshv-miR-K12-3-5p, kshv-miR-K12-4-3p, kshv-miR-K12-4-5p, kshv-miR-K12-5-3p, kshv-miR-K12-5-5p, kshv-miR-K12-6-3p, kshv-miR-K12-6-5p, kshv-miR-K12-7-3p, kshv-miR-K12-7-5p, kshv-miR-K12-8-3p, kshv-miR-K12-8-5p, kshv-miR-K12-9-3p, kshv-miR-K12-9-5p, Ica-miR-125b, lca-miR-15a, lca-miR-16, Ica-miR-17-3p, lca-miR-17-5p, lca-miR-18, Ica-miR-19a, lca-miR-19b, lca-miR-20, lca-miR-216, lca-miR-218, lca-miR-22, lca-miR-23a, lca-miR-27a, lca-miR-92, lco-miR-7642-3p, lgi-let-7, lgi-miR-1, lgi-miR-10, 1gi-miR-100, lgi-miR-1175-3p, lgi-miR-1175-5p, lgi-miR-12, lgi-miR-124, lgi-miR-133-3p, lgi-miR-133-5p, lgi-miR-137, lgi-miR-153, lgi-miR-184, lgi-miR-190, lgi-miR-193, lgi-miR-1984, lgi-miR-1985, lgi-miR-1986, lgi-miR-1988, lgi-miR-1989, lgi-miR-1990, lgi-miR-1991, lgi-miR-1992, 1gi-miR-1993, 1gi-miR-1994a, lgi-miR-1994b, lgi-miR-2001, lgi-miR-216a, lgi-miR-216b, lgi-miR-242a, lgi-miR-242b, lgi-miR-252a, lgi-miR-252b, lgi-miR-263a, lgi-miR-263b, lgi-miR-2722, lgi-miR-278, lgi-miR-279, lgi-miR-281-3p, lgi-miR-281-5p, lgi-miR-29, lgi-miR-2c, lgi-miR-2d, lgi-miR-31, lgi-miR-315, lgi-miR-317, lgi-miR-33-3p, lgi-miR-33-5p, lgi-miR-34, lgi-miR-375, lgi-miR-67, lgi-miR-7, lgi-miR-71, lgi-miR-745a, lgi-miR-745b, lgi-miR-750, 1gi-miR-8, lgi-miR-87, lgi-miR-9-3p, lgi-miR-9-5p, lgi-miR-92, lgi-miR-96a, lgi-miR-96b, lgi-miR-981, lja-miR11066-3p, lja-miR11066-5p, lja-miR11067-3p, lja-miR11067-5p, lja-miR11068a-3p, lja-miR11068a-5p, lja-miR11068b-3p, lja-miR11068c-3p, lja-miR11068c-5p, lja-miR11068d-3p, lja-miR11068e-3p, lja-miR11068f-3p, lja-miR11069-5p, lja-miR11070-5p, lja-miR11071-3p, lja-miR11072a-3p, lja-miR11072b-3p, lja-miR11072b-5p, lja-miR11072c-3p, lja-miR11072d-3p, lja-miR11072e-3p, lja-miR11072f-3p, lja-miR11072g-3p, lja-miR11072 h-3p, lja-miR11072i-3p, lja-miR11072i-5p, lja-miR11072j-3p, lja-miR11072j-5p, lja-miR11073-5p, lja-miR11074-5p, lja-miR11075a-3p, lja-miR11075b-3p, lja-miR11075b-5p, lja-miR11075c-3p, lja-miR11076-3p, lja-miR11076-5p, lja-miR11077a-3p, lja-miR11077a-5p, lja-miR11077b-5p, lja-miR11077c-5p, lja-miR11077d-3p, lja-miR11077d-5p, lja-miR11078a-3p, lja-miR11078b-3p, lja-miR11078c-3p, lja-miR11078c-5p, lja-miR11078d-3p, lja-miR11078e-3p, lja-miR11078f-3p, lja-miR11078g-3p, lja-miR11079-3p, lja-miR11079-5p, lja-miR11080-3p, lja-miR11081-3p, lja-miR11082a-3p, lja-miR11082b-3p, lja-miR11082c-3p, lja-miR11082d-3p, lja-miR11082e-3p, lja-miR11082f-3p, lja-miR11082g-3p, lja-miR11082 h-3p, lja-miR11082i-3p, lja-miR11082i-5p, lja-miR11082j-3p, lja-miR11082k-3p, lja-miR110821-3p, lja-miR11082m-3p, lja-miR11083a-3p, lja-miR11083b-3p, lja-miR11083c-3p, lja-miR11083c-5p, lja-miR11083d-3p, lja-miR11083e-3p, lja-miR11083f-3p, lja-miR11084-5p, lja-miR11085-3p, lja-miR11085-5p, lja-miR11086-3p, lja-miR11086-5p, lja-miR11087-3p, lja-miR11088a-3p, lja-miR11088a-5p, lja-miR11088b-5p, lja-miR11089a-5p, lja-miR11089b-3p, lja-miR11089c-5p, lja-miR11090a-5p, lja-miR11090b-5p, lja-miR11090c-5p, lja-miR11090d-3p, lja-miR11090d-5p, lja-miR11090e-5p, lja-miR11091-3p, lja-miR11092-3p, lja-miR11093-3p, lja-miR11094-3p, lja-miR11095-3p, lja-miR11095-5p, lja-miR11096a-3p, lja-miR11096b-3p, lja-miR11097a-3p, lja-miR11097a-5p, lja-miR11097b-3p, lja-miR11097c-3p, lja-miR11097d-5p, lja-miR11097e-5p, lja-miR11097f-5p, lja-miR11097g-5p, lja-miR11097h-5p, lja-miR11097i-3p, lja-miR11098-3p, lja-miR11099-5p, lja-miR11100-3p, lja-miR11100-5p, lja-miR11101a-3p, lja-miR11101b-3p, lja-miR11102-3p, lja-miR11103a-3p, lja-miR11103b-5p, lja-miR11103c-3p, lja-miR11103c-5p, lja-miR11103d-5p, lja-miR11103e-3p, lja-miR11104-3p, lja-miR11104-5p, lja-miR11105a-5p, lja-miR11105b-5p, lja-miR11105c-5p, lja-miR11105d-5p, lja-miR11106-3p, lja-miR11106-5p, lja-miR11107a-3p, lja-miR11107a-5p, lja-miR11107b-3p, lja-miR11107c-3p, lja-miR11107c-5p, lja-miR11108a-3p, lja-miR11108a-5p, lja-miR11108b-3p, lja-miR11108c-3p, lja-miR11108d-3p, lja-miR11108d-5p, lja-miR11108e-3p, lja-miR11108e-5p, lja-miR11108f-3p, lja-miR11108f-5p, lja-miR11108g-5p, lja-miR11108 h-5p, lja-miR11108i-3p, lja-miR11108j-3p, lja-miR11108j-5p, lja-miR11108k-3p, lja-miR11108k-5p, lja-miR111081-5p, lja-miR11108m-3p, lja-miR11108n-3p, lja-miR11108n-5p, lja-miR111080-3p, lja-miR11108p-3p, lja-miR11108p-5p, lja-miR11108q-3p, lja-miR11108q-5p, lja-miR11108r-3p, lja-miR11108r-5p, lja-miR11108s-3p, lja-miR11108s-5p, lja-miR11108t-3p, lja-miR11108u-3p, lja-miR11108v-3p, lja-miR11108w-5p, lja-miR11108x-5p, lja-miR11109-5p, lja-miR11110a-5p, lja-miR11110b-3p, lja-miR11110b-5p, lja-miR11111-3p, lja-miR11112-3p, lja-miR11113-3p, lja-miR11113-5p, lja-miR11114-3p, lja-miR11114-5p, lja-miR11115-5p, lja-miR11116-3p, lja-miR11117a-3p, lja-miR11117a-5p, lja-miR11117b-3p, lja-miR11118-3p, lja-miR11118-5p, lja-miR11119-5p, lja-miR11120-3p, lja-miR11120-5p, lja-miR11121-3p, lja-miR11122-3p, lja-miR11123-3p, lja-miR11123-5p, lja-miR11124-3p, lja-miR11124-5p, lja-miR11125-5p, lja-miR11126-3p, lja-miR11126-5p, lja-miR11127-5p, lja-miR11128-3p, lja-miR11128-5p, lja-miR11129-3p, lja-miR11130-3p, lja-miR11130-5p, lja-miR11131-3p, lja-miR11131-5p, lja-miR11132-5p, lja-miR11133-5p, lja-miR11134-3p, lja-miR11134-5p, lja-miR11135a-3p, lja-miR11135b-3p, lja-miR11135c-3p, lja-miR11135d-3p, lja-miR11136-3p, lja-miR11136-5p, lja-miR11137-5p, lja-miR11138-3p, lja-miR11139-3p, lja-miR11140-3p, lja-miR11140-5p, lja-miR11141-3p, lja-miR11142-3p, lja-miR11143-3p, lja-miR11143-5p, lja-miR11144-3p, lja-miR11144-5p, lja-miR11145a-3p, lja-miR11145b-3p, lja-miR11145c-3p, lja-miR11145d-3p, lja-miR11146-3p, lja-miR11147-5p, lja-miR11148-5p, lja-miR11149a-3p, lja-miR11149a-5p, lja-miR11149b-3p, lja-miR11150-3p, lja-miR11151-3p, lja-miR11152-5p, lja-miR11153-5p, lja-miR11154-5p, lja-miR11155a-5p, lja-miR11155b-5p, lja-miR11155c-3p, lja-miR11155c-5p, lja-miR11155d-5p, lja-miR11155e-5p, lja-miR11156-5p, lja-miR11157-3p, lja-miR11158-3p, lja-miR11159-3p, lja-miR11160-5p, lja-miR11161-5p, lja-miR11162-3p, lja-miR11163-5p, lja-miR11164-5p, lja-miR11165-3p, lja-miR11166-3p, lja-miR11167-5p, lja-miR11168-5p, lja-miR11169-5p, lja-miR11170-3p, lja-miR11171-5p, lja-miR11172-5p, lja-miR11173-3p, lja-miR11174-3p, lja-miR11175-5p, lja-miR11176-3p, lja-miR11177-5p, lja-miR11178-3p, lja-miR11179-3p, lja-miR11180-3p, lja-miR1120-3p, lja-miR1507a, lja-miR1507b, lja-miR1511-3p, lja-miR164-5p, lja-miR166-3p, lja-miR167, lja-miR167a, lja-miR167b, lja-miR167c, lja-miR168-3p, lja-miR168-5p, lja-miR171a, lja-miR171b, lja-miR171c, lja-miR171d-3p, lja-miR171d-5p, lja-miR172a, lja-miR172b, lja-miR172c, lja-miR2111-3p, lja-miR2111-5p, lja-miR390a-3p, lja-miR390a-5p, lja-miR390b-3p, lja-miR390b-5p, lja-miR395-3p, lja-miR396, lja-miR397, lja-miR398-3p, lja-miR398-5p, lja-miR408, lja-miR477-3p, lja-miR477-5p, lja-miR5287a-3p, lja-miR5287b-3p, lja-miR5287c-3p, lja-miR5287d-3p, lja-miR7516-3p, lja-miR7516-5p, lja-miR7517, lja-miR7518, lja-miR7519, lja-miR7520, lja-miR7521, lja-miR7522, lja-miR7523a, lja-miR7523b, lja-miR7524, lja-miR7525, lja-miR7526a, lja-miR7526b, lja-miR7526c, lja-miR7526d, lja-miR7526e, lja-miR7526f, lja-miR7526g, lja-miR7526h, lja-miR7526i-3p, lja-miR7526j-3p, lja-miR7526k-3p, lja-miR75261-3p, lja-miR75261-5p, lja-miR7527, lja-miR7528, lja-miR7529, lja-miR7530, lja-miR7531, lja-miR7532a, lja-miR7532b, lja-miR7533a, lja-miR7533b, lja-miR7533c-5p, lja-miR7533d-3p, lja-miR7534, lja-miR7535, lja-miR7536a, lja-miR7536b, lja-miR7537, lja-miR7538, lja-miR7539, lja-miR7540a, lja-miR7540b, lja-miR7541, lja-miR7542, lja-miR7543, lja-miR7544, lja-miR7545, lja-miR7546, lla-miR-100, lla-miR-101, lla-miR-103, lla-miR-105, lla-miR-106b, lla-miR-107, lla-miR-124a, lla-miR-125b, lla-miR-127, lla-miR-133a, lla-miR-135, lla-miR-139, lla-miR-143, lla-miR-15a, lla-miR-15b, lla-miR-16, lla-miR-17-3p, lla-miR-17-5p, lla-miR-18, lla-miR-181a-3p, lla-miR-181a-5p, lla-miR-181b, lla-miR-196, lla-miR-198, lla-miR-199a, lla-miR-19a, lla-miR-19b, lla-miR-20, lla-miR-205, lla-miR-218, lla-miR-22, lla-miR-25, lla-miR-26a, lla-miR-28, lla-miR-29a, lla-miR-29b, lla-miR-30b, lla-miR-30c, lla-miR-34a, lla-miR-7, lla-miR-9, lla-miR-92, lla-miR-93, lla-miR-95, lla-miR-99a, Imi-miR-10-3p, lmi-miR-10-5p, lmi-miR-210-3p, lmi-miR-210-5p, lmi-miR-276-3p, lmi-miR-276-5p, lmi-miR-281-3p, lmi-miR-281-5p, lmi-miR-307-3p, lmi-miR-307-5p, Imi-miR-8-3p, lmi-miR-8-5p, lmi-miR-9a-3p, Imi-miR-9a-5p, lus-miR156a, lus-miR156b, lus-miR156c, lus-miR156d, lus-miR156e, lus-miR156f, lus-miR156g, lus-miR156h, lus-miR156i, lus-miR159a, lus-miR159b, lus-miR159c, lus-miR160a, lus-miR160b, lus-miR160c, lus-miR160d, lus-miR160e, lus-miR160f, lus-miR160g, lus-miR160h, lus-miR160i, lus-miR160j, lus-miR162a, lus-miR162b, lus-miR164a, lus-miR164b, lus-miR164c, lus-miR164d, lus-miR164e, lus-miR166a, lus-miR166b, lus-miR166c, lus-miR166d, lus-miR166e, lus-miR166f, lus-miR166g, lus-miR166h, lus-miR166i, lus-miR166j, lus-miR166k, lus-miR167a, lus-miR167b, lus-miR167c, lus-miR167d, lus-miR167e, lus-miR167f, lus-miR167g, lus-miR167h, lus-miR167i, lus-miR168a, lus-miR168b, lus-miR169a, lus-miR169b, lus-miR169c, lus-miR169d, lus-miR169e, lus-miR169f, lus-miR169g, lus-miR169h, lus-miR169i, lus-miR169j, lus-miR169k, lus-miR1691, lus-miR171a, lus-miR171b, lus-miR171c, lus-miR171d, lus-miR171e, lus-miR171f, lus-miR171g, lus-miR171h, lus-miR171i, lus-miR171j, lus-miR172a, lus-miR172b, lus-miR172c, lus-miR172d, lus-miR172e, lus-miR172f, lus-miR172g, lus-miR172h, lus-miR172i, lus-miR172j, lus-miR319a, lus-miR319b, lus-miR390a, lus-miR390b, lus-miR390c, lus-miR390d, lus-miR393a, lus-miR393b, lus-miR393c, lus-miR393d, lus-miR394a, lus-miR394b, lus-miR395a, lus-miR395b, lus-miR395c, lus-miR395d, lus-miR395e, lus-miR396a, lus-miR396b, lus-miR396c, lus-miR396d, lus-miR396e, lus-miR397a, lus-miR397b, lus-miR398a, lus-miR398b, lus-miR398c, lus-miR398d, lus-miR398e, lus-miR398f, lus-miR399a, lus-miR399b, lus-miR399c, lus-miR399d, lus-miR399e, lus-miR399f, lus-miR399g, lus-miR408a, lus-miR530a, lus-miR530b, lus-miR828a, Iva-let-7-5p, lva-miR-1-3p, Iva-miR-10-5p, Iva-miR-124-3p, Iva-miR-125-5p, Iva-miR-133-3p, Iva-miR-137-3p, Iva-miR-153-3p, Iva-miR-182-5p, Iva-miR-183-5p, Iva-miR-184-3p, Iva-miR-193-3p, lva-miR-200-3p, Iva-miR-2001-5p, Iva-miR-2002-3p, Iva-miR-2003-5p, Iva-miR-2004-3p, lva-miR-2005-5p, Iva-miR-2006-3p, Iva-miR-2007-5p, Iva-miR-2008-5p, Iva-miR-2009-3p, lva-miR-2010-3p, Iva-miR-2011-3p, Iva-miR-2012-5p, Iva-miR-2013-3p, Iva-miR-210-3p, Iva-miR-219-5p, Iva-miR-22-3p, Iva-miR-242-5p, Iva-miR-252a-5p, Iva-miR-252b-5p, Iva-miR-278-3p, Iva-miR-29a-3p, Iva-miR-29b-3p, Iva-miR-31-5p, Iva-miR-33-5p, Iva-miR-34-5p, Iva-miR-375-3p, Iva-miR-4847-5p, Iva-miR-4850-5p, Iva-miR-4854-3p, Iva-miR-7-5p, lva-miR-71-5p, Iva-miR-9-5p, Iva-miR-92a-3p, Iva-miR-92b-3p, Iva-miR-96-5p, Iva-miR-981-3p, mcmv-miR-m01-1, mcmv-miR-m01-2-3p, mcmv-miR-m01-2-5p, mcmv-miR-m01-3-3p, mcmv-miR-m01-3-5p, mcmv-miR-m01-4-3p, mcmv-miR-m01-4-5p, mcmv-miR-m107-1-3p, mcmv-miR-m107-1-5p, mcmv-miR-m108-1-3p, mcmv-miR-m108-1-5p, mcmv-miR-m108-2-3p, mcmv-miR-m108-2-5p.1, mcmv-miR-m108-2-5p.2, mcmv-miR-m21-1, mcmv-miR-m22-1, mcmv-miR-M23-1-3p, mcmv-miR-M23-1-5p, mcmv-miR-M23-2-3p, mcmv-miR-M23-2-5p, mcmv-miR-M44-1, mcmv-miR-M55-1, mcmv-miR-m59-1, mcmv-miR-m59-2, mcmv-miR-M87-1, mcmv-miR-m88-1-3p, mcmv-miR-m88-1-5p, mcmv-miR-M95-1-3p, mcmv-miR-M95-1-5p, mco-bantam-3p, mco-let-7-5p, mco-miR-1-3p, mco-miR-10-5p, mco-miR-10293-3p, mco-miR-12065-3p, mco-miR-12066-3p, mco-miR-12067-3p, mco-miR-12068-3p, mco-miR-12068-5p, mco-miR-12069-3p, mco-miR-12070-3p, mco-miR-12071-5p, mco-miR-124a-3p, mco-miR-124b-3p, mco-miR-125-5p, mco-miR-133-3p, mco-miR-153-3p, mco-miR-153-5p, mco-miR-184-3p, mco-miR-190-3p, mco-miR-190-5p, mco-miR-2162-3p, mco-miR-219-5p, mco-miR-277-3p, mco-miR-277b-3p, mco-miR-281-3p, mco-miR-2a-3p, mco-miR-2b-3p, mco-miR-2c-3p, mco-miR-307-3p, mco-miR-31-5p, mco-miR-3479a-3p, mco-miR-3479b-3p, mco-miR-36a-3p, mco-miR-36b-3p, mco-miR-4989-3p, mco-miR-61-3p, mco-miR-71-5p, mco-miR-745-3p, mco-miR-7a-5p, mco-miR-7b-3p, mco-miR-87-3p, mco-miR-9-5p, mco-miR-96-5p, mcpv-miR-P1-3p, mcpv-miR-P1-5p, mdm-miR10978a, mdm-miR10978b, mdm-miR10979, mdm-miR10980a, mdm-miR10980b, mdm-miR10981a, mdm-miR10981b, mdm-miR10981c, mdm-miR10981d, mdm-miR10982a, mdm-miR10982b, mdm-miR10982c, mdm-miR10982d, mdm-miR10983, mdm-miR10984a-3p, mdm-miR10984a-5p, mdm-miR10984b-3p, mdm-miR10984b-5p, mdm-miR10985, mdm-miR10986, mdm-miR10987, mdm-miR10988, mdm-miR10989a, mdm-miR10989b, mdm-miR10989c, mdm-miR10989d, mdm-miR10989e, mdm-miR 10990, mdm-miR10991a, mdm-miR10991b, mdm-miR10991c, mdm-miR10991d, mdm-miR10991e, mdm-miR10992, mdm-miR10993a, mdm-miR10993b, mdm-miR 10993c, mdm-miR10993d, mdm-miR10993e, mdm-miR10993f, mdm-miR10994-3p, mdm-miR10994-5p, mdm-miR10995, mdm-miR10996a, mdm-miR10996b, mdm-miR10997, mdm-miR10998, mdm-miR10999a, mdm-miR10999b, mdm-miR11000, mdm-miR11001, mdm-miR11002a, mdm-miR11002b, mdm-miR11002c-3p, mdm-miR11002c-5p, mdm-miR11003, mdm-miR11004, mdm-miR11005, mdm-miR11006, mdm-miR11007, mdm-miR11008, mdm-miR11009, mdm-miR11010, mdm-miR11011a, mdm-miR11011b, mdm-miR11012a, mdm-miR11012b, mdm-miR11013, mdm-miR11014, mdm-miR11015, mdm-miR11016, mdm-miR11017, mdm-miR11018, mdm-miR11019, mdm-miR11020, mdm-miR1511, mdm-miR156a, mdm-miR156aa, mdm-miR156ab, mdm-miR156ac, mdm-miR156ad, mdm-miR156ae, mdm-miR156b, mdm-miR156c, mdm-miR156d, mdm-miR156e, mdm-miR156f, mdm-miR156g, mdm-miR156h, mdm-miR156i, mdm-miR156j, mdm-miR156k, mdm-miR1561, mdm-miR156m, mdm-miR156n, mdm-miR1560, mdm-miR156p, mdm-miR156q, mdm-miR156r, mdm-miR156s, mdm-miR156t, mdm-miR156u, mdm-miR156v, mdm-miR156w, mdm-miR156x, mdm-miR156y, mdm-miR156z, mdm-miR159a, mdm-miR159b, mdm-miR159c, mdm-miR159d, mdm-miR159e, mdm-miR159f, mdm-miR160a, mdm-miR160b, mdm-miR160c, mdm-miR160d, mdm-miR160e, mdm-miR162a, mdm-miR162b, mdm-miR164a, mdm-miR164b, mdm-miR164c, mdm-miR164d, mdm-miR164e, mdm-miR164f, mdm-miR166a, mdm-miR166b, mdm-miR166c, mdm-miR166d, mdm-miR166e, mdm-miR166f, mdm-miR166g, mdm-miR166h, mdm-miR166i, mdm-miR166j, mdm-miR167a, mdm-miR167b, mdm-miR167c, mdm-miR167d, mdm-miR167e, mdm-miR167f, mdm-miR167g, mdm-miR167h, mdm-miR167i, mdm-miR167j, mdm-miR168a, mdm-miR168b, mdm-miR169a, mdm-miR169b, mdm-miR169c, mdm-miR169d, mdm-miR169e, mdm-miR169f, mdm-miR169g, mdm-miR169h, mdm-miR169i, mdm-miR169j, mdm-miR169k, mdm-miR1691, mdm-miR169m, mdm-miR169n, mdm-miR1690, mdm-miR171a, mdm-miR171b, mdm-miR171c, mdm-miR171d, mdm-miR171e, mdm-miR171f-3p, mdm-miR171f-5p, mdm-miR171g, mdm-miR171h, mdm-miR171i, mdm-miR171j, mdm-miR171k, mdm-miR1711, mdm-miR171m, mdm-miR171n, mdm-miR1710, mdm-miR171p, mdm-miR171q, mdm-miR172a, mdm-miR172b, mdm-miR172c, mdm-miR172d, mdm-miR172e, mdm-miR172f, mdm-miR172g, mdm-miR172h, mdm-miR172i, mdm-miR172j, mdm-miR172k, mdm-miR1721, mdm-miR172m, mdm-miR172n, mdm-miR1720, mdm-miR172p, mdm-miR2111a, mdm-miR2111b, mdm-miR2118a, mdm-miR2118b, mdm-miR2118c, mdm-miR319a, mdm-miR319b-3p, mdm-miR319b-5p, mdm-miR319c-3p, mdm-miR319c-5p, mdm-miR319d, mdm-miR319e, mdm-miR319f, mdm-miR319g, mdm-miR319h, mdm-miR3627a, mdm-miR3627b, mdm-miR3627c, mdm-miR3627d, mdm-miR390a, mdm-miR390b, mdm-miR390c, mdm-miR390d, mdm-miR390e, mdm-miR390f, mdm-miR391, mdm-miR393a, mdm-miR393b, mdm-miR393c, mdm-miR393d, mdm-miR393e, mdm-miR393f, mdm-miR393g, mdm-miR393h, mdm-miR394a, mdm-miR394b, mdm-miR395a, mdm-miR395b, mdm-miR395c, mdm-miR395d-3p, mdm-miR395d-5p, mdm-miR395e, mdm-miR395f, mdm-miR395g-3p, mdm-miR395g-5p, mdm-miR395h, mdm-miR395i-3p, mdm-miR395i-5p, mdm-miR395j, mdm-miR395k, mdm-miR3951, mdm-miR396a, mdm-miR396b, mdm-miR396c, mdm-miR396d, mdm-miR396e, mdm-miR396f, mdm-miR396g, mdm-miR397a, mdm-miR397b, mdm-miR398a, mdm-miR398b, mdm-miR398c, mdm-miR399a, mdm-miR399b, mdm-miR399c, mdm-miR399d, mdm-miR399e, mdm-miR399f, mdm-miR399g, mdm-miR399h, mdm-miR399i, mdm-miR399j, mdm-miR399k, mdm-miR403a, mdm-miR403b, mdm-miR408a, mdm-miR408b, mdm-miR408c, mdm-miR408d, mdm-miR477a, mdm-miR477b, mdm-miR482a-3p, mdm-miR482a-5p, mdm-miR482b, mdm-miR482c, mdm-miR482d, mdm-miR5225a, mdm-miR5225b, mdm-miR5225c, mdm-miR530a, mdm-miR530b, mdm-miR530c, mdm-miR535a, mdm-miR535b, mdm-miR535c, mdm-miR535d, mdm-miR7120a-3p, mdm-miR7120a-5p, mdm-miR7120b-3p, mdm-miR7120b-5p, mdm-miR7121a, mdm-miR7121b, mdm-miR7121c, mdm-miR7121d, mdm-miR7121e, mdm-miR7121f, mdm-miR7121g, mdm-miR7121h, mdm-miR7122a, mdm-miR7122b, mdm-miR7123a, mdm-miR7123b, mdm-miR7124a, mdm-miR7124b, mdm-miR7125, mdm-miR7126-3p, mdm-miR7126-5p, mdm-miR7127a, mdm-miR7127b, mdm-miR7128, mdm-miR827, mdm-miR828a, mdm-miR828b, mdm-miR858, mdo-let-7a-3p, mdo-let-7a-5p, mdo-let-7b, mdo-let-7d, mdo-let-7f-1-3p, mdo-let-7f-2-3p, mdo-let-7f-5p, mdo-let-7g-3p, mdo-let-7g-5p, mdo-let-7i-3p, mdo-let-7i-5p, mdo-miR-1-1-5p, mdo-miR-1-2-5p, mdo-miR-1-3p, mdo-miR-100-3p, mdo-miR-100-5p, mdo-miR-101-1-5p, mdo-miR-101-2-5p, mdo-miR-101-3p, mdo-miR-103-1-5p, mdo-miR-103-2-5p, mdo-miR-103-3p, mdo-miR-106-3p, mdo-miR-106-5p, mdo-miR-107, mdo-miR-10a-3p, mdo-miR-10a-5p, mdo-miR-10b-3p, mdo-miR-10b-5p, mdo-miR-122-3p, mdo-miR-122-5p, mdo-miR-12296-5p, mdo-miR-12297a-3p, mdo-miR-12297a-5p, mdo-miR-12297b-5p, mdo-miR-12297c-3p, mdo-miR-12297c-5p, mdo-miR-12297d-5p, mdo-miR-12297e-3p, mdo-miR-12297f-3p, mdo-miR-12297g-3p, mdo-miR-12297g-5p, mdo-miR-12297 h-3p, mdo-miR-12297i-3p, mdo-miR-12297i-5p, mdo-miR-12297j-3p, mdo-miR-12297j-5p, mdo-miR-12298-3p, mdo-miR-12298-5p, mdo-miR-12299-3p, mdo-miR-12299-5p, mdo-miR-122b-3p, mdo-miR-122b-5p, mdo-miR-12300-3p, mdo-miR-12300-5p, mdo-miR-12301-3p, mdo-miR-12301-5p, mdo-miR-12302-3p, mdo-miR-12302-5p, mdo-miR-12303-3p, mdo-miR-12303-5p, mdo-miR-12304-3p, mdo-miR-12304-5p, mdo-miR-12305-3p, mdo-miR-12305-5p, mdo-miR-12306-3p, mdo-miR-12306-5p, mdo-miR-12307-3p, mdo-miR-12307-5p, mdo-miR-12308a-3p, mdo-miR-12308a-5p, mdo-miR-12308b-3p, mdo-miR-12308b-5p, mdo-miR-12309-3p, mdo-miR-12309-5p, mdo-miR-12310-3p, mdo-miR-12310-5p, mdo-miR-12311-3p, mdo-miR-12311-5p, mdo-miR-12312-3p, mdo-miR-12312-5p, mdo-miR-12313-3p, mdo-miR-12313-5p, mdo-miR-12314-5p, mdo-miR-12315-3p, mdo-miR-12316-3p, mdo-miR-12316-5p, mdo-miR-12317-5p, mdo-miR-12318-3p, mdo-miR-12318-5p, mdo-miR-12319-3p, mdo-miR-12319-5p, mdo-miR-12320-3p, mdo-miR-12320-5p, mdo-miR-12321-3p, mdo-miR-12321-5p, mdo-miR-12322-3p, mdo-miR-12323-3p, mdo-miR-12323-5p, mdo-miR-12324a-3p, mdo-miR-12324a-5p, mdo-miR-12324b-3p, mdo-miR-12324b-5p, mdo-miR-12325-3p, mdo-miR-12325-5p, mdo-miR-12326-3p, mdo-miR-12326-5p, mdo-miR-12327-3p, mdo-miR-12328-3p, mdo-miR-12328-5p, mdo-miR-12329-3p, mdo-miR-12329-5p, mdo-miR-12330-3p, mdo-miR-12330-5p, mdo-miR-12331-3p, mdo-miR-12331-5p, mdo-miR-12332-3p, mdo-miR-12332-5p, mdo-miR-12333-3p, mdo-miR-12333-5p, mdo-miR-12334-3p, mdo-miR-12334-5p, mdo-miR-12335-3p, mdo-miR-12335-5p, mdo-miR-12336-3p, mdo-miR-12336-5p, mdo-miR-12337-3p, mdo-miR-12337-5p, mdo-miR-12338-3p, mdo-miR-12338-5p, mdo-miR-12339-3p, mdo-miR-12339-5p, mdo-miR-12340-3p, mdo-miR-12340-5p, mdo-miR-12341-3p, mdo-miR-12341-5p, mdo-miR-12342-3p, mdo-miR-12342-5p, mdo-miR-12343-3p, mdo-miR-12343-5p, mdo-miR-12344-3p, mdo-miR-12344-5p, mdo-miR-12345-3p, mdo-miR-12346-3p, mdo-miR-12346-5p, mdo-miR-12347-3p, mdo-miR-12347-5p, mdo-miR-12348-3p, mdo-miR-12348-5p, mdo-miR-12349-3p, mdo-miR-12350-5p, mdo-miR-12351-3p, mdo-miR-12351-5p, mdo-miR-12352-3p, mdo-miR-12352-5p, mdo-miR-12353-3p, mdo-miR-12353-5p, mdo-miR-12354-3p, mdo-miR-12354-5p, mdo-miR-12355-3p, mdo-miR-12355-5p, mdo-miR-12356-3p, mdo-miR-12356-5p, mdo-miR-12357-3p, mdo-miR-12357-5p, mdo-miR-12358-3p, mdo-miR-12358-5p, mdo-miR-12359-3p, mdo-miR-12359-5p, mdo-miR-12360-3p, mdo-miR-12360-5p, mdo-miR-12361-3p, mdo-miR-12361-5p, mdo-miR-12362-3p, mdo-miR-12362-5p, mdo-miR-12363-3p, mdo-miR-12363-5p, mdo-miR-12364-3p, mdo-miR-12364-5p, mdo-miR-12365-3p, mdo-miR-12365-5p, mdo-miR-12366a-3p, mdo-miR-12366a-5p, mdo-miR-12366b-3p, mdo-miR-12366b-5p, mdo-miR-12367-3p, mdo-miR-12367-5p, mdo-miR-12368-3p, mdo-miR-12368-5p, mdo-miR-12369-3p, mdo-miR-12369-5p, mdo-miR-12370-3p, mdo-miR-12370-5p, mdo-miR-12371-3p, mdo-miR-12371-5p, mdo-miR-12372-3p, mdo-miR-12372-5p, mdo-miR-12373-5p, mdo-miR-12374-3p, mdo-miR-12374-5p, mdo-miR-12375-3p, mdo-miR-12375-5p, mdo-miR-12376-3p, mdo-miR-12376-5p, mdo-miR-12377-3p, mdo-miR-12378-3p, mdo-miR-12378-5p, mdo-miR-12379-5p, mdo-miR-12380-3p, mdo-miR-12380-5p, mdo-miR-12381-3p, mdo-miR-12381-5p, mdo-miR-12382-3p, mdo-miR-12382-5p, mdo-miR-12383-3p, mdo-miR-12383-5p, mdo-miR-12384-3p, mdo-miR-12384-5p, mdo-miR-12385-3p, mdo-miR-12385-5p, mdo-miR-12386-3p, mdo-miR-12386-5p, mdo-miR-12387-3p, mdo-miR-12387-5p, mdo-miR-12388-3p, mdo-miR-12388-5p, mdo-miR-12389-3p, mdo-miR-12389-5p, mdo-miR-12390-3p, mdo-miR-12390-5p, mdo-miR-12391-3p, mdo-miR-12391-5p, mdo-miR-12392-3p, mdo-miR-12392-5p, mdo-miR-12393-5p, mdo-miR-12394-3p, mdo-miR-12394-5p, mdo-miR-12395-3p, mdo-miR-12395-5p, mdo-miR-12396-3p, mdo-miR-12396-5p, mdo-miR-12397-3p, mdo-miR-12397-5p, mdo-miR-12398-3p, mdo-miR-12398-5p, mdo-miR-12399-3p, mdo-miR-12399-5p, mdo-miR-12400-5p, mdo-miR-12401-3p, mdo-miR-12401-5p, mdo-miR-12402-3p, mdo-miR-12402-5p, mdo-miR-12403-3p, mdo-miR-12403-5p, mdo-miR-12404-3p, mdo-miR-12404-5p, mdo-miR-12405-3p, mdo-miR-12405-5p, mdo-miR-12406-3p, mdo-miR-12406-5p, mdo-miR-12407-3p, mdo-miR-12407-5p, mdo-miR-12408-3p, mdo-miR-12408-5p, mdo-miR-12409-3p, mdo-miR-12409-5p, mdo-miR-12410-5p, mdo-miR-12411-3p, mdo-miR-12412-3p, mdo-miR-12412-5p, mdo-miR-124a-3p, mdo-miR-124a-5p, mdo-miR-1251-3p, mdo-miR-1251-5p, mdo-miR-125b-1-3p, mdo-miR-125b-2-3p, mdo-miR-125b-5p, mdo-miR-126-3p, mdo-miR-126-5p, mdo-miR-128a-3p, mdo-miR-128a-5p, mdo-miR-128b-3p, mdo-miR-128b-5p, mdo-miR-129-3p, mdo-miR-129-5p, mdo-miR-1306-3p, mdo-miR-1306-5p, mdo-miR-130a-3p, mdo-miR-130a-5p, mdo-miR-130b-3p, mdo-miR-130b-5p, mdo-miR-130c-1-5p, mdo-miR-130c-2-5p, mdo-miR-130c-3p, mdo-miR-132-3p, mdo-miR-132-5p, mdo-miR-1329-3p, mdo-miR-1329-5p, mdo-miR-133a-1-5p, mdo-miR-133a-2-5p, mdo-miR-133a-3p, mdo-miR-133b-3p, mdo-miR-133b-5p, mdo-miR-135a-3p, mdo-miR-135a-5p, mdo-miR-135b-3p, mdo-miR-135b-5p, mdo-miR-137a, mdo-miR-137b-3p, mdo-miR-137b-5p, mdo-miR-138, mdo-miR-1388-3p, mdo-miR-1388-5p, mdo-miR-139-3p, mdo-miR-139-5p, mdo-miR-140-3p, mdo-miR-140-5p, mdo-miR-141-3p, mdo-miR-141-5p, mdo-miR-142, mdo-miR-143-3p, mdo-miR-143-5p, mdo-miR-144-3p, mdo-miR-144-5p, mdo-miR-145-3p, mdo-miR-145-5p, mdo-miR-146a-3p, mdo-miR-146a-5p, mdo-miR-146b-3p, mdo-miR-146b-5p, mdo-miR-147b, mdo-miR-148-3p, mdo-miR-148-5p, mdo-miR-150-3p, mdo-miR-150-5p, mdo-miR-151, mdo-miR-152-3p, mdo-miR-152-5p, mdo-miR-153-1-5p, mdo-miR-153-2-5p, mdo-miR-153-3p, mdo-miR-1540a, mdo-miR-1540b-3p, mdo-miR-1540b-5p, mdo-miR-1541, mdo-miR-1542-1-5p, mdo-miR-1542-2-5p, mdo-miR-1542-3p, mdo-miR-1543, mdo-miR-1544a-3p, mdo-miR-1544a-5p, mdo-miR-1544b-3p, mdo-miR-1544b-5p, mdo-miR-1544c-3p, mdo-miR-1544c-5p, mdo-miR-1544d-3p, mdo-miR-1544d-5p, mdo-miR-1544e-3p, mdo-miR-1544e-5p, mdo-miR-1544f-3p, mdo-miR-1544f-5p, mdo-miR-1544g-3p, mdo-miR-1544g-5p, mdo-miR-1545a-3p, mdo-miR-1545a-5p, mdo-miR-1545b-3-3p, mdo-miR-1545b-3p, mdo-miR-1545b-5p, mdo-miR-1545c-3p, mdo-miR-1545c-5p, mdo-miR-1545d-3-5p, mdo-miR-1545d-3p, mdo-miR-1545d-4-5p, mdo-miR-1545d-5p, mdo-miR-1545e-3p, mdo-miR-1545e-5p, mdo-miR-1545f-3p, mdo-miR-1545f-5p, mdo-miR-1545g-3-5p, mdo-miR-1545g-3p, mdo-miR-1545g-5p, mdo-miR-1546-3p, mdo-miR-1546-5p, mdo-miR-1546b-3p, mdo-miR-1546b-5p, mdo-miR-1547-3p, mdo-miR-1547-5p, mdo-miR-1548-3p, mdo-miR-1548-5p, mdo-miR-1549-3p, mdo-miR-1549-5p, mdo-miR-1549b-3p, mdo-miR-1549b-5p, mdo-miR-15a-3p, mdo-miR-15a-5p, mdo-miR-15c-3p, mdo-miR-15c-5p, mdo-miR-16-1-3p, mdo-miR-16-2-3p, mdo-miR-16-5p, mdo-miR-1643a-3p, mdo-miR-1643a-5p, mdo-miR-1643b-3p, mdo-miR-1643b-5p, mdo-miR-16b-3p, mdo-miR-16b-5p, mdo-miR-17-3p, mdo-miR-17-5p, mdo-miR-1805-3p, mdo-miR-1805-5p, mdo-miR-181a-1-3p, mdo-miR-181a-2-3p, mdo-miR-181a-3-3p, mdo-miR-181a-5p, mdo-miR-181b-1-3p, mdo-miR-181b-2-3p, mdo-miR-181b-3-3p, mdo-miR-181b-5p, mdo-miR-182-3p, mdo-miR-182-5p, mdo-miR-183-3p, mdo-miR-183-5p, mdo-miR-184-3p, mdo-miR-184-5p, mdo-miR-186-3p, mdo-miR-186-5p, mdo-miR-187-3p, mdo-miR-187-5p, mdo-miR-18a-3p, mdo-miR-18a-5p, mdo-miR-18b-3p, mdo-miR-18b-5p, mdo-miR-190a-3p, mdo-miR-190a-5p, mdo-miR-190b, mdo-miR-191-3p, mdo-miR-191-5p, mdo-miR-193a-3p, mdo-miR-193a-5p, mdo-miR-193b-3p, mdo-miR-193b-5p, mdo-miR-195, mdo-miR-196a-3p, mdo-miR-196a-5p, mdo-miR-196b, mdo-miR-199b-1-5p, mdo-miR-199b-2-5p, mdo-miR-199b-3-5p, mdo-miR-199b-3p, mdo-miR-19a-3p, mdo-miR-19a-5p, mdo-miR-19b-1-5p, mdo-miR-19b-2-5p, mdo-miR-19b-3p, mdo-miR-200a-3p, mdo-miR-200a-5p, mdo-miR-200b-3p, mdo-miR-200b-5p, mdo-miR-200c-3p, mdo-miR-200c-5p, mdo-miR-203, mdo-miR-204, mdo-miR-205a, mdo-miR-205b-3p, mdo-miR-205b-5p, mdo-miR-206, mdo-miR-208a, mdo-miR-208b-3p, mdo-miR-208b-5p, mdo-miR-20a-3p, mdo-miR-20a-5p, mdo-miR-20b-3p, mdo-miR-20b-5p, mdo-miR-21-3p, mdo-miR-21-5p, mdo-miR-210-3p, mdo-miR-210-5p, mdo-miR-210b-3p, mdo-miR-210b-5p, mdo-miR-212-3p, mdo-miR-212-5p, mdo-miR-214-3p, mdo-miR-214-5p, mdo-miR-215, mdo-miR-216, mdo-miR-217, mdo-miR-218-1-3p, mdo-miR-218-2-3p, mdo-miR-218-5p, mdo-miR-219-1-3p, mdo-miR-219-2-3p, mdo-miR-219-5p, mdo-miR-22-3p, mdo-miR-22-5p, mdo-miR-221-3p, mdo-miR-221-5p, mdo-miR-222a, mdo-miR-223-3p, mdo-miR-223-5p, mdo-miR-23a-3p, mdo-miR-23a-5p, mdo-miR-23b-3p, mdo-miR-23b-5p, mdo-miR-24-3p, mdo-miR-24-5p, mdo-miR-25, mdo-miR-26-1-3p, mdo-miR-26-2-3p, mdo-miR-26-3-3p, mdo-miR-26-5p, mdo-miR-27a-3p, mdo-miR-27a-5p, mdo-miR-27b-3p, mdo-miR-27b-5p, mdo-miR-28, mdo-miR-2970-3p, mdo-miR-2970-5p, mdo-miR-2985-2-3p, mdo-miR-2985-3p, mdo-miR-2985-5p, mdo-miR-29a-1-5p, mdo-miR-29a-2-5p, mdo-miR-29a-3p, mdo-miR-29b-1-5p, mdo-miR-29b-2-5p, mdo-miR-29b-3p, mdo-miR-301-3p, mdo-miR-301-5p, mdo-miR-302a, mdo-miR-302b, mdo-miR-302c, mdo-miR-302d, mdo-miR-30a-3p, mdo-miR-30a-5p, mdo-miR-30b-3p, mdo-miR-30b-5p, mdo-miR-30c-1-3p, mdo-miR-30c-2-3p, mdo-miR-30c-5p, mdo-miR-30d-3p, mdo-miR-30d-5p, mdo-miR-30e-3p, mdo-miR-30e-5p, mdo-miR-31-3p, mdo-miR-31-5p, mdo-miR-32-3p, mdo-miR-32-5p, mdo-miR-325-3p, mdo-miR-325-5p, mdo-miR-33-3p, mdo-miR-33-5p, mdo-miR-338, mdo-miR-340-3p, mdo-miR-340-5p, mdo-miR-34a-3p, mdo-miR-34a-5p, mdo-miR-34b-3p, mdo-miR-34b-5p, mdo-miR-34c-3p, mdo-miR-34c-5p, mdo-miR-34d-3p, mdo-miR-34d-5p, mdo-miR-352-3p, mdo-miR-352-5p, mdo-miR-3600-3p, mdo-miR-363, mdo-miR-365-3p, mdo-miR-365-5p, mdo-miR-367, mdo-miR-375, mdo-miR-383-3p, mdo-miR-383-5p, mdo-miR-425-3p, mdo-miR-425-5p, mdo-miR-429, mdo-miR-449a-3p, mdo-miR-449a-5p, mdo-miR-449b-3p, mdo-miR-449b-5p, mdo-miR-449c-3p, mdo-miR-449c-5p, mdo-miR-451, mdo-miR-455-3p, mdo-miR-455-5p, mdo-miR-460-3p, mdo-miR-460-5p, mdo-miR-497-3p, mdo-miR-497-5p, mdo-miR-499-3p, mdo-miR-499-5p, mdo-miR-505-3p, mdo-miR-505-5p, mdo-miR-551a, mdo-miR-551b-3p, mdo-miR-551b-5p, mdo-miR-551c-3p, mdo-miR-551c-5p, mdo-miR-599-3p, mdo-miR-599-5p, mdo-miR-7, mdo-miR-7244-3p, mdo-miR-7244-5p, mdo-miR-7245-3p, mdo-miR-7245-5p, mdo-miR-7246-3p, mdo-miR-7246-5p, mdo-miR-7247-3p, mdo-miR-7247-5p, mdo-miR-7248-3p, mdo-miR-7248-5p, mdo-miR-7249-3p, mdo-miR-7249-5p, mdo-miR-7250-3p, mdo-miR-7250-5p, mdo-miR-7251-3p, mdo-miR-7251-5p, mdo-miR-7252-3p, mdo-miR-7252-5p, mdo-miR-7253-3p, mdo-miR-7253-5p, mdo-miR-7254-3p, mdo-miR-7254-5p, mdo-miR-7255-3p, mdo-miR-7255-5p, mdo-miR-7256-3p, mdo-miR-7256-5p, mdo-miR-7257-3p, mdo-miR-7257-5p, mdo-miR-7258-3p, mdo-miR-7258-5p, mdo-miR-7259-3p, mdo-miR-7259-5p, mdo-miR-7260-3p, mdo-miR-7260-5p, mdo-miR-7261-3p, mdo-miR-7261-5p, mdo-miR-7262a-3p, mdo-miR-7262a-5p, mdo-miR-7262b-3p, mdo-miR-7262b-5p, mdo-miR-7262c-3p, mdo-miR-7262c-5p, mdo-miR-7262d-3p, mdo-miR-7262d-5p, mdo-miR-7262e-3p, mdo-miR-7262e-5p, mdo-miR-7262f-3p, mdo-miR-7262f-5p, mdo-miR-7262g-3p, mdo-miR-7262g-5p, mdo-miR-7262 h-3p, mdo-miR-7262 h-5p, mdo-miR-7262i-3p, mdo-miR-7262i-5p, mdo-miR-7262j-3p, mdo-miR-7262j-5p, mdo-miR-7262k-3p, mdo-miR-7262k-5p, mdo-miR-72621-3p, mdo-miR-72621-5p, mdo-miR-7262m-3p, mdo-miR-7263-3p, mdo-miR-7263-5p, mdo-miR-7264-3p, mdo-miR-7264-5p, mdo-miR-7265-3p, mdo-miR-7265-5p, mdo-miR-7266-3p, mdo-miR-7266-5p, mdo-miR-7267-3p, mdo-miR-7267-5p, mdo-miR-7268-3p, mdo-miR-7268-5p, mdo-miR-7269-3p, mdo-miR-7269-5p, mdo-miR-7270-3p, mdo-miR-7270-5p, mdo-miR-7271-3p, mdo-miR-7271-5p, mdo-miR-7272-3p, mdo-miR-7272-5p, mdo-miR-7273-3p, mdo-miR-7273-5p, mdo-miR-7274-3p, mdo-miR-7274-5p, mdo-miR-7275-3p, mdo-miR-7275-5p, mdo-miR-7276-3p, mdo-miR-7276-5p, mdo-miR-7277-3p, mdo-miR-7277-5p, mdo-miR-7278-3p, mdo-miR-7278-5p, mdo-miR-7279-3p, mdo-miR-7279-5p, mdo-miR-7280-3p, mdo-miR-7280-5p, mdo-miR-7281-3p, mdo-miR-7281-5p, mdo-miR-7282-3p, mdo-miR-7282-5p, mdo-miR-7283-3p, mdo-miR-7283-5p, mdo-miR-7284-3p, mdo-miR-7284-5p, mdo-miR-7284b-3p, mdo-miR-7284b-5p, mdo-miR-7285-3p, mdo-miR-7285-5p, mdo-miR-7286-3p, mdo-miR-7286-5p, mdo-miR-7286b-3p, mdo-miR-7286b-5p, mdo-miR-7287-3p, mdo-miR-7287-5p, mdo-miR-7288-3p, mdo-miR-7288-5p, mdo-miR-7289-3p, mdo-miR-7289-5p, mdo-miR-7290-3p, mdo-miR-7290-5p, mdo-miR-7291-3p, mdo-miR-7291-5p, mdo-miR-7292-3p, mdo-miR-7292-5p, mdo-miR-7293-3p, mdo-miR-7293-5p, mdo-miR-7294-3p, mdo-miR-7294-5p, mdo-miR-7295-3p, mdo-miR-7295-5p, mdo-miR-7296-3p, mdo-miR-7296-5p, mdo-miR-7297-3p, mdo-miR-7297-5p, mdo-miR-7298-3p, mdo-miR-7298-5p, mdo-miR-7299-3p, mdo-miR-7299-5p, mdo-miR-7300-3p, mdo-miR-7300-5p, mdo-miR-7301-3p, mdo-miR-7301-5p, mdo-miR-7302-3p, mdo-miR-7302-5p, mdo-miR-7303-3p, mdo-miR-7303-5p, mdo-miR-7304-3p, mdo-miR-7304-5p, mdo-miR-7305-3p, mdo-miR-7305-5p, mdo-miR-7306-3p, mdo-miR-7306-5p, mdo-miR-7307-3p, mdo-miR-7307-5p, mdo-miR-7308-3p, mdo-miR-7308-5p, mdo-miR-7309-3p, mdo-miR-7309-5p, mdo-miR-7310-3p, mdo-miR-7310-5p, mdo-miR-7311-3p, mdo-miR-7311-5p, mdo-miR-7312-3p, mdo-miR-7312-5p, mdo-miR-7312b-3p, mdo-miR-7312b-5p, mdo-miR-7313-3p, mdo-miR-7313-5p, mdo-miR-7314-3p, mdo-miR-7314-5p, mdo-miR-7315-3p, mdo-miR-7315-5p, mdo-miR-7316-3p, mdo-miR-7316-5p, mdo-miR-7317-3p, mdo-miR-7317-5p, mdo-miR-7318-3p, mdo-miR-7318-5p, mdo-miR-7319-3p, mdo-miR-7319-5p, mdo-miR-7320-3p, mdo-miR-7320-5p, mdo-miR-7320b-5p, mdo-miR-7321-3p, mdo-miR-7321-5p, mdo-miR-7322-3p, mdo-miR-7322-5p, mdo-miR-7323-3p, mdo-miR-7323-5p, mdo-miR-7323b-3p, mdo-miR-7323b-5p, mdo-miR-7324-3p, mdo-miR-7324-5p, mdo-miR-7325-3p, mdo-miR-7325-5p, mdo-miR-7326-3p, mdo-miR-7326-5p, mdo-miR-7327-3p, mdo-miR-7327-5p, mdo-miR-7328-3p, mdo-miR-7328-5p, mdo-miR-7329-3p, mdo-miR-7329-5p, mdo-miR-7330-3p, mdo-miR-7330-5p, mdo-miR-7331-1-3p, mdo-miR-7331-2-3p, mdo-miR-7331-5p, mdo-miR-7332-3p, mdo-miR-7332-5p, mdo-miR-7333-3p, mdo-miR-7333-5p, mdo-miR-7334-3p, mdo-miR-7334-5p, mdo-miR-7335-3p, mdo-miR-7335-5p, mdo-miR-7336-3p, mdo-miR-7336-5p, mdo-miR-7337-3p, mdo-miR-7337-5p, mdo-miR-7338-3p, mdo-miR-7338-5p, mdo-miR-7339-3p, mdo-miR-7339-5p, mdo-miR-7340-3p, mdo-miR-7340-5p, mdo-miR-7341-3p, mdo-miR-7341-5p, mdo-miR-7342-3p, mdo-miR-7342-5p, mdo-miR-7343-3p, mdo-miR-7343-5p, mdo-miR-7344-3p, mdo-miR-7344-5p, mdo-miR-7345-3p, mdo-miR-7345-5p, mdo-miR-7345b-3p, mdo-miR-7345b-5p, mdo-miR-7346-3p, mdo-miR-7346-5p, mdo-miR-7347-3p, mdo-miR-7347-5p, mdo-miR-7348-3p, mdo-miR-7348-5p, mdo-miR-7349-3p, mdo-miR-7349-5p, mdo-miR-7350-3p, mdo-miR-7350-5p, mdo-miR-7351-3p, mdo-miR-7351-5p, mdo-miR-7352-3p, mdo-miR-7352-5p, mdo-miR-7353-3p, mdo-miR-7353-5p, mdo-miR-7354-3p, mdo-miR-7354-5p, mdo-miR-7355-3p, mdo-miR-7355-5p, mdo-miR-7356-3p, mdo-miR-7356-5p, mdo-miR-7356b-3p, mdo-miR-7356b-5p, mdo-miR-7357-3p, mdo-miR-7357-5p, mdo-miR-7358-3p, mdo-miR-7358-5p, mdo-miR-7359-5p, mdo-miR-7359a-3p, mdo-miR-7359b-3p, mdo-miR-7360-3p, mdo-miR-7360-5p, mdo-miR-7360-9-5p, mdo-miR-7361-3p, mdo-miR-7361-5p, mdo-miR-7362-3p, mdo-miR-7362-5p, mdo-miR-7363-3p, mdo-miR-7363-5p, mdo-miR-7364-3p, mdo-miR-7364-5p, mdo-miR-7365-3p, mdo-miR-7365-5p, mdo-miR-7366-3p, mdo-miR-7366-5p, mdo-miR-7367-3p, mdo-miR-7367-5p, mdo-miR-7368-3p, mdo-miR-7368-5p, mdo-miR-7369-3p, mdo-miR-7369-5p, mdo-miR-7370-3p, mdo-miR-7370-5p, mdo-miR-7370i-3p, mdo-miR-7370i-5p, mdo-miR-7371a-3p, mdo-miR-7371a-5p, mdo-miR-7371b-3p, mdo-miR-7371b-5p, mdo-miR-7371c-3p, mdo-miR-7371c-5p, mdo-miR-7371d-3p, mdo-miR-7371d-5p, mdo-miR-7371e-3p, mdo-miR-7371e-5p, mdo-miR-7371f-3p, mdo-miR-7371f-5p, mdo-miR-7371g-3p, mdo-miR-7371g-5p, mdo-miR-7371 h-3p, mdo-miR-7371 h-5p, mdo-miR-7371j-3p, mdo-miR-7371j-5p, mdo-miR-7371k-3p, mdo-miR-7371k-5p, mdo-miR-73711-3p, mdo-miR-73711-5p, mdo-miR-7371m-3p, mdo-miR-7371m-5p, mdo-miR-7371n-3p, mdo-miR-7371n-5p, mdo-miR-73710-3p, mdo-miR-73710-5p, mdo-miR-7371p-3p, mdo-miR-7371p-5p, mdo-miR-7371q-3p, mdo-miR-7371q-5p, mdo-miR-7372-3p, mdo-miR-7372-5p, mdo-miR-7373a-3p, mdo-miR-7373a-5p, mdo-miR-7373b-3p, mdo-miR-7373b-5p, mdo-miR-7373c-3p, mdo-miR-7373c-5p, mdo-miR-7373d-3p, mdo-miR-7373d-5p, mdo-miR-7373e-3p, mdo-miR-7373e-5p, mdo-miR-7373f-3p, mdo-miR-7373f-5p, mdo-miR-7374a-3p, mdo-miR-7374a-5p, mdo-miR-7374b-3p, mdo-miR-7374b-5p, mdo-miR-7374c-3p, mdo-miR-7374c-5p, mdo-miR-7374d-3p, mdo-miR-7374d-5p, mdo-miR-7375-3p, mdo-miR-7375-5p, mdo-miR-7375c-3p, mdo-miR-7375c-4-5p, mdo-miR-7375c-5p, mdo-miR-7375d-3p, mdo-miR-7375d-5p, mdo-miR-7375e-3p, mdo-miR-7375e-5p, mdo-miR-7376-3p, mdo-miR-7376-5p, mdo-miR-7377-3p, mdo-miR-7377-5p, mdo-miR-7378-3p, mdo-miR-7378-5p, mdo-miR-7379-3p, mdo-miR-7379-5p, mdo-miR-7380-3p, mdo-miR-7380-5p, mdo-miR-7381-3p, mdo-miR-7381-5p, mdo-miR-7382-3p, mdo-miR-7382-5p, mdo-miR-7383-3p, mdo-miR-7383-5p, mdo-miR-7384-3p, mdo-miR-7384-5p, mdo-miR-7385a-3p, mdo-miR-7385a-5p, mdo-miR-7385b-3p, mdo-miR-7385b-5p, mdo-miR-7385c-3p, mdo-miR-7385c-5p, mdo-miR-7385d-3p, mdo-miR-7385d-5p, mdo-miR-7385e-3p, mdo-miR-7385e-5p, mdo-miR-7385f-3p, mdo-miR-7385f-5p, mdo-miR-7385g-2-3p, mdo-miR-7385g-3-3p, mdo-miR-7385g-3p, mdo-miR-7385g-5p, mdo-miR-7385 h-3p, mdo-miR-7385 h-5p, mdo-miR-7385i-2-3p, mdo-miR-7385i-3p, mdo-miR-73851-5p, mdo-miR-7386a-3-3p, mdo-miR-7386a-3p, mdo-miR-7386a-5p, mdo-miR-7386b-3p, mdo-miR-7386b-5p, mdo-miR-7386c-3p, mdo-miR-7386c-5p, mdo-miR-7386d-3p, mdo-miR-7386d-5p, mdo-miR-7386e-3p, mdo-miR-7386e-5p, mdo-miR-7386f-3p, mdo-miR-7386f-5p, mdo-miR-7386g-3p, mdo-miR-7386g-5p, mdo-miR-7386 h-3p, mdo-miR-7386 h-5p, mdo-miR-7386i-3p, mdo-miR-7386i-5p, mdo-miR-7386j-3p, mdo-miR-7386j-5p, mdo-miR-7386k-3p, mdo-miR-7386k-5p, mdo-miR-73861-3p, mdo-miR-73861-5p, mdo-miR-7386m-3p, mdo-miR-7386m-5p, mdo-miR-7386n-3p, mdo-miR-7386n-5p, mdo-miR-73860-3p, mdo-miR-73860-5p, mdo-miR-7386p-3p, mdo-miR-7386p-5p, mdo-miR-7386q-3p, mdo-miR-7386q-5p, mdo-miR-7387-3p, mdo-miR-7387-5p, mdo-miR-7388a-3p, mdo-miR-7388a-5p, mdo-miR-7388b-3p, mdo-miR-7388b-5p, mdo-miR-7388c-3p, mdo-miR-7388c-5p, mdo-miR-7389-3p, mdo-miR-7389-5p, mdo-miR-7390-3p, mdo-miR-7390-5p, mdo-miR-7391-3p, mdo-miR-7391-5p, mdo-miR-7392-3p, mdo-miR-7392-5p, mdo-miR-7393-3p, mdo-miR-7393-5p, mdo-miR-7394a-3p, mdo-miR-7394a-5p, mdo-miR-7394b-3p, mdo-miR-7394b-5p, mdo-miR-7394c-3p, mdo-miR-7394c-5p, mdo-miR-7395-3p, mdo-miR-7395-5p, mdo-miR-7396-3p, mdo-miR-7396-5p, mdo-miR-7397-3p, mdo-miR-7397-5p, mdo-miR-7398a-1-5p, mdo-miR-7398a-3p, mdo-miR-7398a-4-5p, mdo-miR-7398a-5-5p, mdo-miR-7398a-5p, mdo-miR-7398b-3p, mdo-miR-7398b-5p, mdo-miR-7398c-3p, mdo-miR-7398c-5p, mdo-miR-7398d-3p, mdo-miR-7398d-5p, mdo-miR-7398e-3p, mdo-miR-7398e-5p, mdo-miR-7398f-3p, mdo-miR-7398f-5p, mdo-miR-7398g-3p, mdo-miR-7398g-5p, mdo-miR-7398 h-3p, mdo-miR-7398 h-5p, mdo-miR-7398i-3p, mdo-miR-7398i-5p, mdo-miR-7398j-3p, mdo-miR-7398j-5p, mdo-miR-7398k-1-3p, mdo-miR-7398k-2-3p, mdo-miR-7398k-5p, mdo-miR-73981-3p, mdo-miR-73981-5p, mdo-miR-7398m-3p, mdo-miR-7398m-5p, mdo-miR-7398n-3p, mdo-miR-7398n-5p, mdo-miR-73980-3p, mdo-miR-73980-5p, mdo-miR-7398p-3p, mdo-miR-7398p-5p, mdo-miR-7398q-3p, mdo-miR-7398q-5p, mdo-miR-7398r-3p, mdo-miR-7398r-5p, mdo-miR-7398s-3p, mdo-miR-7398s-5p, mdo-miR-7398t-3p, mdo-miR-7398t-5p, mdo-miR-7398u-3p, mdo-miR-7398u-5p, mdo-miR-7398v-3p, mdo-miR-7398v-5p, mdo-miR-7398w-3p, mdo-miR-7398w-5p, mdo-miR-7398x-3p, mdo-miR-7398y-3p, mdo-miR-7398y-5p, mdo-miR-7399-3p, mdo-miR-7399-5p, mdo-miR-759, mdo-miR-761, mdo-miR-873-3p, mdo-miR-873-5p, mdo-miR-875-3p, mdo-miR-875-5p, mdo-miR-885, mdo-miR-92a-1-5p, mdo-miR-92a-2-5p, mdo-miR-92a-3p, mdo-miR-92b-3p, mdo-miR-92b-5p, mdo-miR-93-3p, mdo-miR-93-5p, mdo-miR-96, mdo-miR-9a-3p, mdo-miR-9a-5p, mdo-miR-9b-3p, mdo-miR-9b-5p, mdo-miR-9c-3p, mdo-miR-9c-5p, mdv1-miR-M1-3p, mdv1-miR-M1-5p, mdv1-miR-M10-3p, mdv1-miR-M10-5p, mdv1-miR-M11-3p, mdv1-miR-M11-5p, mdv1-miR-M12-3p, mdv1-miR-M12-5p, mdv1-miR-M13, mdv1-miR-M2-3p, mdv1-miR-M2-5p, mdv1-miR-M3-3p, mdv1-miR-M3-5p, mdv1-miR-M31, mdv1-miR-M4-3p, mdv1-miR-M4-5p, mdv1-miR-M5-3p, mdv1-miR-M5-5p, mdv1-miR-M6-3p, mdv1-miR-M6-5p, mdv1-miR-M7-3p, mdv1-miR-M7-5p, mdv1-miR-M8-3p, mdv1-miR-M8-5p, mdv1-miR-M9-3p, mdv1-miR-M9-5p, mdv2-miR-M14-3p, mdv2-miR-M14-5p, mdv2-miR-M15-3p, mdv2-miR-M15-5p, mdv2-miR-M16-3p, mdv2-miR-M16-5p, mdv2-miR-M17-3p, mdv2-miR-M17-5p, mdv2-miR-M18-3p, mdv2-miR-M18-5p, mdv2-miR-M19-3p, mdv2-miR-M19-5p, mdv2-miR-M20-3p, mdv2-miR-M20-5p, mdv2-miR-M21-3p, mdv2-miR-M21-5p, mdv2-miR-M22-3p, mdv2-miR-M22-5p, mdv2-miR-M23-3p, mdv2-miR-M23-5p, mdv2-miR-M24-3p, mdv2-miR-M24-5p, mdv2-miR-M25-3p, mdv2-miR-M25-5p, mdv2-miR-M26-3p, mdv2-miR-M26-5p, mdv2-miR-M27-3p, mdv2-miR-M27-5p, mdv2-miR-M28-3p, mdv2-miR-M28-5p, mdv2-miR-M29-3p, mdv2-miR-M29-5p, mdv2-miR-M30-3p, mdv2-miR-M30-5p, mdv2-miR-M32-3p, mdv2-miR-M32-5p, mes-miR11891, mes-miR11892, mes-miR1446, mes-miR1446b, mes-miR156a, mes-miR156b, mes-miR156c, mes-miR156d, mes-miR156e, mes-miR156f, mes-miR156g, mes-miR156h, mes-miR156i, mes-miR156j, mes-miR156k, mes-miR159a-3p, mes-miR159a-5p, mes-miR159b, mes-miR159c, mes-miR159d, mes-miR160a, mes-miR160b, mes-miR160c, mes-miR160d, mes-miR160e, mes-miR160f, mes-miR160g, mes-miR160h, mes-miR162, mes-miR164a, mes-miR164b, mes-miR164c, mes-miR 164d, mes-miR166a, mes-miR166b, mes-miR166c, mes-miR166d, mes-miR166e, mes-miR166f, mes-miR166g, mes-miR166h, mes-miR166i, mes-miR166j, mes-miR167a, mes-miR167b, mes-miR167c, mes-miR167d, mes-miR167e, mes-miR167f, mes-miR167g, mes-miR167h, mes-miR168a, mes-miR169a, mes-miR169aa, mes-miR169ab, mes-miR169ac, mes-miR169ad, mes-miR169b, mes-miR169c, mes-miR169d, mes-miR169e, mes-miR169f, mes-miR169g, mes-miR169h, mes-miR169i, mes-miR169j, mes-miR169k, mes-miR1691, mes-miR169m, mes-miR169n, mes-miR1690, mes-miR169p, mes-miR169q, mes-miR169r, mes-miR169s, mes-miR169t, mes-miR169u, mes-miR169v, mes-miR 169w, mes-miR169x, mes-miR169y, mes-miR169z, mes-miR171a, mes-miR171b, mes-miR171c, mes-miR171d, mes-miR171e, mes-miR171f, mes-miR171g, mes-miR171h, mes-miR171i, mes-miR171j, mes-miR171k, mes-miR1711, mes-miR172a, mes-miR172b, mes-miR172c, mes-miR172d, mes-miR172e, mes-miR172f, mes-miR2111a, mes-miR2111b, mes-miR2118, mes-miR2275, mes-miR2950, mes-miR319a, mes-miR319b, mes-miR319c, mes-miR319d, mes-miR319e, mes-miR319f, mes-miR319g, mes-miR319h, mes-miR3627, mes-miR390, mes-miR390b, mes-miR393a, mes-miR393b, mes-miR393c, mes-miR393d, mes-miR394a, mes-miR394b, mes-miR394c, mes-miR395a, mes-miR395b, mes-miR395c, mes-miR395d, mes-miR395e, mes-miR396a, mes-miR396b, mes-miR396c, mes-miR396d, mes-miR396e, mes-miR396f, mes-miR397, mes-miR397b, mes-miR398, mes-miR399a, mes-miR399b, mes-miR399c, mes-miR399d, mes-miR399e, mes-miR399f, mes-miR399g, mes-miR399h, mes-miR403a, mes-miR403b, mes-miR408, mes-miR477a, mes-miR477b, mes-miR477c, mes-miR477d, mes-miR477e, mes-miR477f, mes-miR477g, mes-miR477h, mes-miR477i, mes-miR477j, mes-miR477k, mes-miR482, mes-miR482b, mes-miR482c, mes-miR482d, mes-miR482e, mes-miR530a, mes-miR530b, mes-miR535a, mes-miR535b, mes-miR535c, mes-miR535d, mes-miR6445, mes-miR827, mes-miR828a, mes-miR828b, mes-miR9386, meu-miR-15c, meu-miR-16c, meu-miR-6313, mghv-miR-M1-1-3p, mghv-miR-M1-1-5p, mghv-miR-M1-10-3p, mghv-miR-M1-10-5p, mghv-miR-M1-11-3p, mghv-miR-M1-11-5p, mghv-miR-M1-12-3p, mghv-miR-M1-12-5p, mghv-miR-M1-13-3p, mghv-miR-M1-13-5p, mghv-miR-M1-14-3p, mghv-miR-M1-14-5p, mghv-miR-M1-15, mghv-miR-M1-2-3p, mghv-miR-M1-2-5p, mghv-miR-M1-3-3p, mghv-miR-M1-3-5p, mghv-miR-M1-4-3p, mghv-miR-M1-4-5p, mghv-miR-M1-5-3p, mghv-miR-M1-5-5p, mghv-miR-M1-6-3p, mghv-miR-M1-6-5p, mghv-miR-M1-7-3p, mghv-miR-M1-7-5p, mghv-miR-M1-8-3p, mghv-miR-M1-8-5p, mghv-miR-M1-9, mja-miR-6489-3p, mja-miR-6489-5p, mja-miR-6491, mja-miR-6492, mja-miR-6493-3p, mja-miR-6493-5p, mja-miR-6494, mle-bantam-3p, mle-bantam-5p, mle-let-7-3p, mle-let-7-5p, mle-miR-1-3p, mle-miR-1-5p, mle-miR-100-3p, mle-miR-100-5p, mle-miR-10a-3p, mle-miR-10a-5p, mle-miR-10b-3p, mle-miR-10b-5p, mle-miR-1175-3p, mle-miR-1175-5p, mle-miR-12-3p, mle-miR-12-5p, mle-miR-12096a-3p, mle-miR-12096a-5p, mle-miR-12096b-3p, mle-miR-12096b-5p, mle-miR-12097-3p, mle-miR-12097-5p, mle-miR-12098-3p, mle-miR-12098-5p, mle-miR-12099-3p, mle-miR-12099-5p, mle-miR-12100-3p, mle-miR-12100-5p, mle-miR-12101-2-c5p, mle-miR-12101-3p, mle-miR-12101-5p, mle-miR-12102-3p, mle-miR-12102-5p, mle-miR-12103-3p, mle-miR-12104-3p, mle-miR-124-3p, mle-miR-124-5p, mle-miR-125-3p, mle-miR-125-5p, mle-miR-133-3p, mle-miR-133-5p, mle-miR-137-3p, mle-miR-137-5p, mle-miR-153-3p, mle-miR-153-5p, mle-miR-184-3p, mle-miR-184-5p, mle-miR-190-3p, mle-miR-190-5p, mle-miR-193-3p, mle-miR-193-5p, mle-miR-1984-3p, mle-miR-1984-5p, mle-miR-1985-3p, mle-miR-1985-5p, mle-miR-1986-3p, mle-miR-1986-5p, mle-miR-1988a-3p, mle-miR-1988a-5p, mle-miR-1988b-3p, mle-miR-1988b-5p, mle-miR-1989a-3p, mle-miR-1989a-5p, mle-miR-1989b-3p, mle-miR-1989b-5p, mle-miR-1990-3p, mle-miR-1990-5p, mle-miR-1991-3p, mle-miR-1991-5p, mle-miR-1992-3p, mle-miR-1992-5p, mle-miR-1993-3p, mle-miR-1993-5p, mle-miR-1994a-3p, mle-miR-1994a-5p, mle-miR-1994b-3p, mle-miR-1994b-5p, mle-miR-2001-3p, mle-miR-2001-5p, mle-miR-210-3p, mle-miR-210-5p, mle-miR-216a-3p, mle-miR-216a-5p, mle-miR-216b-3p, mle-miR-216b-5p, mle-miR-219-3p, mle-miR-219-5p, mle-miR-242-3p, mle-miR-242-5p, mle-miR-252a-3p, mle-miR-252a-5p, mle-miR-252b-3p, mle-miR-252b-5p, mle-miR-263a-3p, mle-miR-263a-5p, mle-miR-263b-3p, mle-miR-263b-5p, mle-miR-2722-3p, mle-miR-2722-5p, mle-miR-277-3p, mle-miR-277-5p, mle-miR-279-3p, mle-miR-279-5p, mle-miR-281-3p, mle-miR-281-5p, mle-miR-29a-3p, mle-miR-29a-5p, mle-miR-29b-3p, mle-miR-29b-5p, mle-miR-29c-3p, mle-miR-29c-5p, mle-miR-2a-3p, mle-miR-2a-5p, mle-miR-2b-3p, mle-miR-2b-5p, mle-miR-2c-3p, mle-miR-2c-5p, mle-miR-2d-3p, mle-miR-2d-5p, mle-miR-2e-3p, mle-miR-2e-5p, mle-miR-2f-3p, mle-miR-2f-5p, mle-miR-2g-3p, mle-miR-2g-5p, mle-miR-2 h-3p, mle-miR-2 h-5p, mle-miR-2i-3p, mle-miR-2i-5p, mle-miR-2j-3p, mle-miR-2j-5p, mle-miR-2k-3p, mle-miR-2k-5p, mle-miR-31-3p, mle-miR-31-5p, mle-miR-315-3p, mle-miR-315-5p, mle-miR-317-3p, mle-miR-317-5p, mle-miR-33-3p, mle-miR-34-3p, mle-miR-34-5p, mle-miR-365-3p, mle-miR-365-5p, mle-miR-375-3p, mle-miR-375-5p, mle-miR-67-3p, mle-miR-67-5p, mle-miR-7-3p, mle-miR-7-5p, mle-miR-71-3p, mle-miR-71-5p, mle-miR-745a-3p, mle-miR-745a-5p, mle-miR-745b-3p, mle-miR-745b-5p, mle-miR-750-3p, mle-miR-750-5p, mle-miR-8-3p, mle-miR-8-5p, mle-miR-87-3p, mle-miR-87-5p, mle-miR-9-3p, mle-miR-9-5p, mle-miR-92a-3p, mle-miR-92a-5p, mle-miR-92b-3p, mle-miR-92b-5p, mle-miR-92c-3p, mle-miR-92c-5p, mle-miR-96a-3p, mle-miR-96a-5p, mle-miR-96b-3p, mle-miR-96b-5p, mle-miR-981-3p, mle-miR-981-5p, mml-let-7a-2-3p, mml-let-7a-3p, mml-let-7a-5p, mml-let-7b-3p, mml-let-7b-5p, mml-let-7c-3p, mml-let-7c-5p, mml-let-7d, mml-let-7e-3p, mml-let-7e-5p, mml-let-7f-3p, mml-let-7f-5p, mml-let-7g-3p, mml-let-7g-5p, mml-let-7i-3p, mml-let-7i-5p, mml-miR-1-1-5p, mml-miR-1-2-5p, mml-miR-1-3p, mml-miR-100-3p, mml-miR-100-5p, mml-miR-101-1-5p, mml-miR-101-2-5p, mml-miR-101-3p, mml-miR-103-3p, mml-miR-103-5p, mml-miR-105-3p, mml-miR-105-5p, mml-miR-106a-3p, mml-miR-106a-5p, mml-miR-106b-3p, mml-miR-106b-5p, mml-miR-107-3p, mml-miR-107-5p, mml-miR-10a-3p, mml-miR-10a-5p, mml-miR-10b-3p, mml-miR-10b-5p, mml-miR-1179, mml-miR-1180-3p, mml-miR-1180-5p, mml-miR-1185-3p, mml-miR-1185-5p, mml-miR-1224-3p, mml-miR-1224-5p, mml-miR-1225-3p, mml-miR-1225-5p, mml-miR-1226, mml-miR-1227, mml-miR-122a-3p, mml-miR-122a-5p, mml-miR-1230, mml-miR-1232, mml-miR-1233, mml-miR-1234, mml-miR-1235, mml-miR-1239, mml-miR-1240, mml-miR-1241, mml-miR-1243-3p, mml-miR-1243-5p, mml-miR-1245-3p, mml-miR-1245-5p, mml-miR-1247-3p, mml-miR-1247-5p, mml-miR-1249, mml-miR-124a-3p, mml-miR-124a-5p, mml-miR-1250, mml-miR-1251-3p, mml-miR-1251-5p, mml-miR-1255a-3p, mml-miR-1255a-5p, mml-miR-1255b, mml-miR-1258, mml-miR-125a-3p, mml-miR-125a-5p, mml-miR-125b-1-3p, mml-miR-125b-2-3p, mml-miR-125b-5p, mml-miR-126, mml-miR-1260b, mml-miR-1262-3p, mml-miR-1262-5p, mml-miR-127-3p, mml-miR-127-5p, mml-miR-1271-3p, mml-miR-1271-5p, mml-miR-1277, mml-miR-1283, mml-miR-1284, mml-miR-128a-3p, mml-miR-128a-5p, mml-miR-128b-3p, mml-miR-128b-5p, mml-miR-129-1-3p, mml-miR-129-2-3p, mml-miR-129-5p, mml-miR-1296-3p, mml-miR-1296-5p, mml-miR-1298-3p, mml-miR-1298-5p, mml-miR-1304, mml-miR-1306-3p, mml-miR-1306-5p, mml-miR-130a-3p, mml-miR-130a-5p, mml-miR-130b-3p, mml-miR-130b-5p, mml-miR-132-3p, mml-miR-132-5p, mml-miR-1323-3p, mml-miR-1323-5p, mml-miR-133a, mml-miR-133b-3p, mml-miR-133b-5p, mml-miR-133c-3p, mml-miR-133c-5p, mml-miR-134-3p, mml-miR-134-5p, mml-miR-135a-1-3p, mml-miR-135a-2-3p, mml-miR-135a-5p, mml-miR-135b-3p, mml-miR-135b-5p, mml-miR-136, mml-miR-137-3p, mml-miR-137-5p, mml-miR-138-3p, mml-miR-138-5p, mml-miR-139-3p, mml-miR-139-5p, mml-miR-140-3p, mml-miR-140-5p, mml-miR-141-3p, mml-miR-141-5p, mml-miR-142-3p, mml-miR-142-5p, mml-miR-143-3p, mml-miR-143-5p, mml-miR-144, mml-miR-145-3p, mml-miR-145-5p, mml-miR-146a-3p, mml-miR-146a-5p, mml-miR-146b-3p, mml-miR-146b-5p, mml-miR-147a, mml-miR-147b, mml-miR-148a-3p, mml-miR-148a-5p, mml-miR-148b-3p, mml-miR-148b-5p, mml-miR-149-3p, mml-miR-149-5p, mml-miR-150-3p, mml-miR-150-5p, mml-miR-151-3p, mml-miR-151-5p, mml-miR-152-3p, mml-miR-152-5p, mml-miR-153-1-5p, mml-miR-153-2-5p, mml-miR-153-3p, mml-miR-154-3p, mml-miR-154-5p, mml-miR-155, mml-miR-15a-3p, mml-miR-15a-5p, mml-miR-15b-3p, mml-miR-15b-5p, mml-miR-16-1-3p, mml-miR-16-2-3p, mml-miR-16-5p, mml-miR-17-3p, mml-miR-17-5p, mml-miR-181a-3p, mml-miR-181a-5p, mml-miR-181b-1-3p, mml-miR-181b-2-3p, mml-miR-181b-5p, mml-miR-181c-3p, mml-miR-181c-5p, mml-miR-181d, mml-miR-182, mml-miR-183-3p, mml-miR-183-5p, mml-miR-184, mml-miR-185-3p, mml-miR-185-5p, mml-miR-186-3p, mml-miR-186-5p, mml-miR-187-3p, mml-miR-187-5p, mml-miR-188-3p, mml-miR-188-5p, mml-miR-18a-3p, mml-miR-18a-5p, mml-miR-18b, mml-miR-190a-3p, mml-miR-190a-5p, mml-miR-190b, mml-miR-191-3p, mml-miR-191-5p, mml-miR-1911-3p, mml-miR-1911-5p, mml-miR-192-3p, mml-miR-192-5p, mml-miR-193a-3p, mml-miR-193a-5p, mml-miR-193b-3p, mml-miR-193b-5p, mml-miR-194-3p, mml-miR-194-5p, mml-miR-195-3p, mml-miR-195-5p, mml-miR-196a-3p, mml-miR-196a-5p, mml-miR-196b-3p, mml-miR-196b-5p, mml-miR-197-3p, mml-miR-197-5p, mml-miR-198, mml-miR-199a, mml-miR-199a-3p, mml-miR-199a-5p, mml-miR-19a-3p, mml-miR-19a-5p, mml-miR-19b, mml-miR-200a-3p, mml-miR-200a-5p, mml-miR-200c-3p, mml-miR-200c-5p, mml-miR-203, mml-miR-204-3p, mml-miR-204-5p, mml-miR-205, mml-miR-206, mml-miR-208a-3p, mml-miR-208a-5p, mml-miR-208b-3p, mml-miR-208b-5p, mml-miR-20a-3p, mml-miR-20a-5p, mml-miR-20b-3p, mml-miR-20b-5p, mml-miR-21-3p, mml-miR-21-5p, mml-miR-210-3p, mml-miR-210-5p, mml-miR-211, mml-miR-2114, mml-miR-212-3p, mml-miR-212-5p, mml-miR-214-3p, mml-miR-214-5p, mml-miR-215-3p, mml-miR-215-5p, mml-miR-216a-3p, mml-miR-216a-5p, mml-miR-216b, mml-miR-217, mml-miR-218-1-3p, mml-miR-218-2-3p, mml-miR-218-5p, mml-miR-219, mml-miR-219-3p, mml-miR-219-5p, mml-miR-22, mml-miR-221-3p, mml-miR-221-5p, mml-miR-222-3p, mml-miR-222-5p, mml-miR-223, mml-miR-224-3p, mml-miR-224-5p, mml-miR-23a-3p, mml-miR-23a-5p, mml-miR-23b-3p, mml-miR-23b-5p, mml-miR-24-3p, mml-miR-24-5p, mml-miR-25, mml-miR-26a-1-3p, mml-miR-26a-2-3p, mml-miR-26a-5p, mml-miR-26b-3p, mml-miR-26b-5p, mml-miR-27a-3p, mml-miR-27a-5p, mml-miR-27b-3p, mml-miR-27b-5p, mml-miR-28-3p, mml-miR-28-5p, mml-miR-296-3p, mml-miR-296-5p, mml-miR-297, mml-miR-298, mml-miR-299-3p, mml-miR-299-5p, mml-miR-29a-3p, mml-miR-29a-5p, mml-miR-29b-1-5p, mml-miR-29b-2-5p, mml-miR-29b-3p, mml-miR-29c-3p, mml-miR-29c-5p, mml-miR-301a-3p, mml-miR-301a-5p, mml-miR-301b, mml-miR-302a-3p, mml-miR-302a-5p, mml-miR-302b, mml-miR-302c, mml-miR-302d, mml-miR-3059-3p, mml-miR-3059-5p, mml-miR-30a-3p, mml-miR-30a-5p, mml-miR-30b-3p, mml-miR-30b-5p, mml-miR-30c-1-3p, mml-miR-30c-2-3p, mml-miR-30c-5p, mml-miR-30d-3p, mml-miR-30d-5p, mml-miR-30e-3p, mml-miR-30e-5p, mml-miR-31-3p, mml-miR-31-5p, mml-miR-3122, mml-miR-3140, mml-miR-3145-3p, mml-miR-3145-5p, mml-miR-3146, mml-miR-3151, mml-miR-3154, mml-miR-3167-3p, mml-miR-3167-5p, mml-miR-3173, mml-miR-32-3p, mml-miR-32-5p, mml-miR-3200-3p, mml-miR-3200-5p, mml-miR-320a, mml-miR-320b, mml-miR-320c, mml-miR-323a-3p, mml-miR-323a-5p, mml-miR-323b-3p, mml-miR-323b-5p, mml-miR-324-3p, mml-miR-324-5p, mml-miR-325, mml-miR-328, mml-miR-329-1-5p, mml-miR-329-2-5p, mml-miR-329-3p, mml-miR-330-3p, mml-miR-330-5p, mml-miR-331-3p, mml-miR-331-5p, mml-miR-335-3p, mml-miR-335-5p, mml-miR-337-3p, mml-miR-337-5p, mml-miR-338-3p, mml-miR-338-5p, mml-miR-339-3p, mml-miR-339-5p, mml-miR-33a, mml-miR-33b-3p, mml-miR-33b-5p, mml-miR-340-3p, mml-miR-340-5p, mml-miR-342-3p, mml-miR-342-5p, mml-miR-345, mml-miR-346, mml-miR-34a-3p, mml-miR-34a-5p, mml-miR-34b, mml-miR-34c-3p, mml-miR-34c-5p, mml-miR-3577-3p, mml-miR-3577-5p, mml-miR-361-3p, mml-miR-361-5p, mml-miR-362-3p, mml-miR-362-5p, mml-miR-363-3p, mml-miR-363-5p, mml-miR-365-1-5p, mml-miR-365-2-5p, mml-miR-365-3p, mml-miR-367, mml-miR-369-3p, mml-miR-369-5p, mml-miR-370-3p, mml-miR-370-5p, mml-miR-371-3p, mml-miR-371-5p, mml-miR-372-3p, mml-miR-372-5p, mml-miR-373, mml-miR-374a-3p, mml-miR-374a-5p, mml-miR-374b-3p, mml-miR-374b-5p, mml-miR-375, mml-miR-376a-1-5p, mml-miR-376a-2-5p, mml-miR-376a-3p, mml-miR-376b-3p, mml-miR-376b-5p, mml-miR-376c-3p, mml-miR-376c-5p, mml-miR-377-3p, mml-miR-377-5p, mml-miR-378a, mml-miR-378d, mml-miR-378j, mml-miR-379-3p, mml-miR-379-5p, mml-miR-380-3p, mml-miR-380-5p, mml-miR-381-3p, mml-miR-381-5p, mml-miR-382-3p, mml-miR-382-5p, mml-miR-383, mml-miR-384, mml-miR-3927, mml-miR-3938-3p, mml-miR-3938-5p, mml-miR-409-3p, mml-miR-409-5p, mml-miR-410-3p, mml-miR-410-5p, mml-miR-411-3p, mml-miR-411-5p, mml-miR-412-3p, mml-miR-412-5p, mml-miR-421, mml-miR-422a, mml-miR-423-3p, mml-miR-423-5p, mml-miR-424-3p, mml-miR-424-5p, mml-miR-425, mml-miR-429-3p, mml-miR-429-5p, mml-miR-431, mml-miR-432-3p, mml-miR-432-5p, mml-miR-433-3p, mml-miR-433-5p, mml-miR-4446-3p, mml-miR-4446-5p, mml-miR-448, mml-miR-449a-3p, mml-miR-449a-5p, mml-miR-449b-3p, mml-miR-449b-5p, mml-miR-450a-1-3p, mml-miR-450a-2-3p, mml-miR-450a-5p, mml-miR-450b-3p, mml-miR-450b-5p, mml-miR-451, mml-miR-452-3p, mml-miR-452-5p, mml-miR-4536, mml-miR-454-3p, mml-miR-454-5p, mml-miR-455-3p, mml-miR-455-5p, mml-miR-4650, mml-miR-4667, mml-miR-4677-3p, mml-miR-4677-5p, mml-miR-4703-3p, mml-miR-4703-5p, mml-miR-4716, mml-miR-4743, mml-miR-4766-3p, mml-miR-4766-5p, mml-miR-4796-3p, mml-miR-4796-5p, mml-miR-4803, mml-miR-484, mml-miR-485-3p, mml-miR-485-5p, mml-miR-486-3p, mml-miR-486-5p, mml-miR-487a, mml-miR-487b-3p, mml-miR-487b-5p, mml-miR-488-3p, mml-miR-488-5p, mml-miR-489-3p, mml-miR-489-5p, mml-miR-490-3p, mml-miR-490-5p, mml-miR-491-3p, mml-miR-491-5p, mml-miR-492, mml-miR-493-3p, mml-miR-493-5p, mml-miR-494-3p, mml-miR-494-5p, mml-miR-495-3p, mml-miR-495-5p, mml-miR-496, mml-miR-497-3p, mml-miR-497-5p, mml-miR-498, mml-miR-499-3p, mml-miR-499-5p, mml-miR-500a-3p, mml-miR-500a-5p, mml-miR-500b-3p, mml-miR-500b-5p, mml-miR-501-3p, mml-miR-501-5p, mml-miR-502-3p, mml-miR-502-5p, mml-miR-503-3p, mml-miR-503-5p, mml-miR-504-3p, mml-miR-504-5p, mml-miR-505-3p, mml-miR-505-5p, mml-miR-506-3p, mml-miR-506-5p, mml-miR-507, mml-miR-508-3p, mml-miR-508-5p, mml-miR-509, mml-miR-510-3p, mml-miR-510-5p, mml-miR-511-3p, mml-miR-511-5p, mml-miR-512-3p, mml-miR-512-5p, mml-miR-513a, mml-miR-513b, mml-miR-514a-3p, mml-miR-514a-5p, mml-miR-514b-3p, mml-miR-514b-5p, mml-miR-516a-3p, mml-miR-516a-5p, mml-miR-516b, mml-miR-517a, mml-miR-517b, mml-miR-517c, mml-miR-518a-3p, mml-miR-518a-5p, mml-miR-518b, mml-miR-518c-3p, mml-miR-518c-5p, mml-miR-518d-3p, mml-miR-518d-5p, mml-miR-518e, mml-miR-518f, mml-miR-518g-3p, mml-miR-518g-5p, mml-miR-519a, mml-miR-519a-3p, mml-miR-519a-5p, mml-miR-519b, mml-miR-519c, mml-miR-519d, mml-miR-519e, mml-miR-520a-3p, mml-miR-520a-5p, mml-miR-520g, mml-miR-521, mml-miR-523a, mml-miR-523b, mml-miR-524-3p, mml-miR-524-5p, mml-miR-525, mml-miR-526b, mml-miR-532-3p, mml-miR-532-5p, mml-miR-539, mml-miR-541-3p, mml-miR-541-5p, mml-miR-542-3p, mml-miR-542-5p, mml-miR-543-3p, mml-miR-543-5p, mml-miR-544, mml-miR-545, mml-miR-548a, mml-miR-548b, mml-miR-548c, mml-miR-548d-3p, mml-miR-548d-5p, mml-miR-548e, mml-miR-548f, mml-miR-548g-3p, mml-miR-548g-5p, mml-miR-548 h-3p, mml-miR-548 h-5p, mml-miR-548i-3p, mml-miR-548i-5p, mml-miR-548j-3p, mml-miR-548j-5p, mml-miR-549a, mml-miR-549b-3p, mml-miR-549b-5p, mml-miR-550-3p, mml-miR-550-5p, mml-miR-551a, mml-miR-551b-3p, mml-miR-551b-5p, mml-miR-552, mml-miR-553, mml-miR-554, mml-miR-556-3p, mml-miR-556-5p, mml-miR-557, mml-miR-558, mml-miR-562, mml-miR-563, mml-miR-567, mml-miR-568, mml-miR-569, mml-miR-5697-3p, mml-miR-5697-5p, mml-miR-570, mml-miR-571, mml-miR-572, mml-miR-573, mml-miR-574, mml-miR-576-3p, mml-miR-576-5p, mml-miR-577-3p, mml-miR-577-5p, mml-miR-578, mml-miR-579, mml-miR-580, mml-miR-581, mml-miR-582-3p, mml-miR-582-5p, mml-miR-583, mml-miR-584-3p, mml-miR-584-5p, mml-miR-586, mml-miR-587, mml-miR-589, mml-miR-590-3p, mml-miR-590-5p, mml-miR-592-3p, mml-miR-592-5p, mml-miR-593, mml-miR-595, mml-miR-597, mml-miR-598-3p, mml-miR-598-5p, mml-miR-599-3p, mml-miR-599-5p, mml-miR-600, mml-miR-601, mml-miR-603, mml-miR-604, mml-miR-605, mml-miR-607, mml-miR-609, mml-miR-611, mml-miR-612, mml-miR-6127, mml-miR-6128, mml-miR-6129, mml-miR-6130, mml-miR-6131, mml-miR-6132, mml-miR-6133, mml-miR-6134, mml-miR-615-3p, mml-miR-615-5p, mml-miR-616, mml-miR-618, mml-miR-619, mml-miR-622, mml-miR-624, mml-miR-625, mml-miR-626, mml-miR-627-3p, mml-miR-627-5p, mml-miR-628-3p, mml-miR-628-5p, mml-miR-631, mml-miR-632, mml-miR-633, mml-miR-636, mml-miR-638, mml-miR-639, mml-miR-640, mml-miR-642, mml-miR-643, mml-miR-644, mml-miR-648, mml-miR-649, mml-miR-6505-3p, mml-miR-6505-5p, mml-miR-650a, mml-miR-650b, mml-miR-650c, mml-miR-650d, mml-miR-651, mml-miR-652, mml-miR-6529-3p, mml-miR-6529-5p, mml-miR-653-3p, mml-miR-653-5p, mml-miR-654-3p, mml-miR-654-5p, mml-miR-656-3p, mml-miR-656-5p, mml-miR-657, mml-miR-660-3p, mml-miR-660-5p, mml-miR-661, mml-miR-662, mml-miR-663, mml-miR-664, mml-miR-665, mml-miR-668, mml-miR-670-3p, mml-miR-670-5p, mml-miR-671-3p, mml-miR-671-5p, mml-miR-675-3p, mml-miR-675-5p, mml-miR-676-3p, mml-miR-676-5p, mml-miR-6790-3p, mml-miR-6790-5p, mml-miR-6794-3p, mml-miR-6794-5p, mml-miR-6827-3p, mml-miR-6827-5p, mml-miR-7, mml-miR-708-3p, mml-miR-708-5p, mml-miR-711, mml-miR-7163-3p, mml-miR-7163-5p, mml-miR-7164-3p, mml-miR-7164-5p, mml-miR-7165-3p, mml-miR-7165-5p, mml-miR-7166-3p, mml-miR-7166-5p, mml-miR-7167-3p, mml-miR-7167-5p, mml-miR-7168-3p, mml-miR-7168-5p, mml-miR-7169-3p, mml-miR-7169-5p, mml-miR-7170-3p, mml-miR-7170-5p, mml-miR-7171-3p, mml-miR-7171-5p, mml-miR-7172-3p, mml-miR-7172-5p, mml-miR-7173-3p, mml-miR-7173-5p, mml-miR-7174-3p, mml-miR-7174-5p, mml-miR-7175-3p, mml-miR-7175-5p, mml-miR-7176-3p, mml-miR-7176-5p, mml-miR-7177-3p, mml-miR-7177-5p, mml-miR-7178-3p, mml-miR-7178-5p, mml-miR-7179-3p, mml-miR-7179-5p, mml-miR-7180-3p, mml-miR-7180-5p, mml-miR-7181-3p, mml-miR-7181-5p, mml-miR-7182-3p, mml-miR-7182-5p, mml-miR-7183-3p, mml-miR-7183-5p, mml-miR-7184-3p, mml-miR-7184-5p, mml-miR-7185-3p, mml-miR-7185-5p, mml-miR-7186-3p, mml-miR-7186-5p, mml-miR-7187-3p, mml-miR-7187-5p, mml-miR-7188-3p, mml-miR-7188-5p, mml-miR-7189-3p, mml-miR-7189-5p, mml-miR-7190-3p, mml-miR-7190-5p, mml-miR-7191-3p, mml-miR-7191-5p, mml-miR-7192-3p, mml-miR-7192-5p, mml-miR-7193-3p, mml-miR-7193-5p, mml-miR-7194-3p, mml-miR-7194-5p, mml-miR-7195-3p, mml-miR-7195-5p, mml-miR-7196-3p, mml-miR-7196-5p, mml-miR-7197-3p, mml-miR-7197-5p, mml-miR-7198-3p, mml-miR-7198-5p, mml-miR-7199-3p, mml-miR-7199-5p, mml-miR-7200-3p, mml-miR-7200-5p, mml-miR-7201-3p, mml-miR-7201-5p, mml-miR-7202-3p, mml-miR-7202-5p, mml-miR-7203-3p, mml-miR-7203-5p, mml-miR-7204-3p, mml-miR-7204-5p, mml-miR-7205-3p, mml-miR-7205-5p, mml-miR-7206-3p, mml-miR-7206-5p, mml-miR-7207-3p, mml-miR-7207-5p, mml-miR-7208-3p, mml-miR-7208-5p, mml-miR-758-3p, mml-miR-758-5p, mml-miR-759, mml-miR-760, mml-miR-761, mml-miR-762, mml-miR-764, mml-miR-765, mml-miR-767-3p, mml-miR-767-5p, mml-miR-769-3p, mml-miR-769-5p, mml-miR-770-5p, mml-miR-802, mml-miR-873-3p, mml-miR-873-5p, mml-miR-874-3p, mml-miR-874-5p, mml-miR-875-3p, mml-miR-875-5p, mml-miR-876-3p, mml-miR-876-5p, mml-miR-877-3p, mml-miR-877-5p, mml-miR-885-3p, mml-miR-885-5p, mml-miR-887, mml-miR-888, mml-miR-889-3p, mml-miR-889-5p, mml-miR-890-3p, mml-miR-890-5p, mml-miR-891a, mml-miR-891b-3p, mml-miR-891b-5p, mml-miR-892a, mml-miR-892b, mml-miR-892c-3p, mml-miR-892c-5p, mml-miR-9-3-3p, mml-miR-9-3p, mml-miR-9-5p, mml-miR-920, mml-miR-922, mml-miR-924, mml-miR-92a-3p, mml-miR-92a-5p, mml-miR-92b-3p, mml-miR-92b-5p, mml-miR-93-3p, mml-miR-93-5p, mml-miR-933, mml-miR-934-3p, mml-miR-934-5p, mml-miR-936, mml-miR-937, mml-miR-938, mml-miR-939, mml-miR-940, mml-miR-942-3p, mml-miR-942-5p, mml-miR-944, mml-miR-95-3p, mml-miR-95-5p, mml-miR-96, mml-miR-98, mml-miR-99a-3p, mml-miR-99a-5p, mml-miR-99b-3p, mml-miR-99b-5p, mmr-let-7a, mmr-let-7b, mmr-let-7c, mmr-let-7f, mmr-let-7g, mmr-miR-10, mmr-miR-100, mmr-miR-101, mmr-miR-103a, mmr-miR-103b, mmr-miR-107, mmr-miR-10a, mmr-miR-125a, mmr-miR-126, mmr-miR-128, mmr-miR-1307, mmr-miR-130a, mmr-miR-132, mmr-miR-136, mmr-miR-138, mmr-miR-1388, mmr-miR-140, mmr-miR-145, mmr-miR-146a, mmr-miR-146b, mmr-miR-148b, mmr-miR-151, mmr-miR-152, mmr-miR-15a, mmr-miR-17, mmr-miR-18, mmr-miR-181a, mmr-miR-182, mmr-miR-1839, mmr-miR-186, mmr-miR-187, mmr-miR-188, mmr-miR-191, mmr-miR-193, mmr-miR-195, mmr-miR-196b, mmr-miR-197, mmr-miR-199, mmr-miR-19b, mmr-miR-20, mmr-miR-204, mmr-miR-21, mmr-miR-211, mmr-miR-212, mmr-miR-214, mmr-miR-218a, mmr-miR-218b, mmr-miR-22, mmr-miR-221, mmr-miR-222, mmr-miR-23b, mmr-miR-24, mmr-miR-26a, mmr-miR-26b, mmr-miR-27b, mmr-miR-28, mmr-miR-296, mmr-miR-29a, mmr-miR-29b, mmr-miR-29c, mmr-miR-301a, mmr-miR-30a, mmr-miR-30c, mmr-miR-30e, mmr-miR-31, mmr-miR-320a, mmr-miR-324, mmr-miR-328, mmr-miR-340, mmr-miR-342, mmr-miR-345, mmr-miR-34a, mmr-miR-34b, mmr-miR-34c, mmr-miR-361, mmr-miR-365, mmr-miR-374a, mmr-miR-381, mmr-miR-3959, mmr-miR-409, mmr-miR-410, mmr-miR-411a, mmr-miR-411b, mmr-miR-424, mmr-miR-425, mmr-miR-455, mmr-miR-484, mmr-miR-486, mmr-miR-497, mmr-miR-500, mmr-miR-502a, mmr-miR-502b, mmr-miR-532, mmr-miR-542, mmr-miR-590, mmr-miR-615, mmr-miR-6313, mmr-miR-660, mmr-miR-708, mmr-miR-872, mmr-miR-92a, mmr-miR-92b, mmr-miR-98, mmr-miR-99a, mmu-let-7a-1-3p, mmu-let-7a-2-3p, mmu-let-7a-5p, mmu-let-7b-3p, mmu-let-7b-5p, mmu-let-7c-1-3p, mmu-let-7c-2-3p, mmu-let-7c-5p, mmu-let-7d-3p, mmu-let-7d-5p, mmu-let-7e-3p, mmu-let-7e-5p, mmu-let-7f-1-3p, mmu-let-7f-2-3p, mmu-let-7f-5p, mmu-let-7g-3p, mmu-let-7g-5p, mmu-let-7i-3p, mmu-let-7i-5p, mmu-let-7j, mmu-let-7k, mmu-miR-100-3p, mmu-miR-100-5p, mmu-miR-101a-3p, mmu-miR-101a-5p, mmu-miR-101b-3p, mmu-miR-101b-5p, mmu-miR-101c, mmu-miR-103-1-5p, mmu-miR-103-2-5p, mmu-miR-103-3p, mmu-miR-105, mmu-miR-106a-3p, mmu-miR-106a-5p, mmu-miR-106b-3p, mmu-miR-106b-5p, mmu-miR-107-3p, mmu-miR-107-5p, mmu-miR-10a-3p, mmu-miR-10a-5p, mmu-miR-10b-3p, mmu-miR-10b-5p, mmu-miR-1187, mmu-miR-1188-3p, mmu-miR-1188-5p, mmu-miR-1190, mmu-miR-1191a, mmu-miR-1191b-3p, mmu-miR-1191b-5p, mmu-miR-1192, mmu-miR-1193-3p, mmu-miR-1193-5p, mmu-miR-1194, mmu-miR-1195, mmu-miR-1197-3p, mmu-miR-1197-5p, mmu-miR-1198-3p, mmu-miR-1198-5p, mmu-miR-1199-3p, mmu-miR-1199-5p, mmu-miR-12178-3p, mmu-miR-12178-5p, mmu-miR-12179-5p, mmu-miR-12180-3p, mmu-miR-12181-3p, mmu-miR-12181-5p, mmu-miR-12182-3p, mmu-miR-12182-5p, mmu-miR-12183-5p, mmu-miR-12184-3p, mmu-miR-12184-5p, mmu-miR-12185-3p, mmu-miR-12185-5p, mmu-miR-12186-3p, mmu-miR-12186-5p, mmu-miR-12187-3p, mmu-miR-12187-5p, mmu-miR-12188-3p, mmu-miR-12188-5p, mmu-miR-12189-3p, mmu-miR-12190-3p, mmu-miR-12191-3p, mmu-miR-12191-5p, mmu-miR-12192-5p, mmu-miR-12193-3p, mmu-miR-12193-5p, mmu-miR-12194-3p, mmu-miR-12195-3p, mmu-miR-12195-5p, mmu-miR-12196-3p, mmu-miR-12196-5p, mmu-miR-12197-3p, mmu-miR-12198-3p, mmu-miR-12199-3p, mmu-miR-122-3p, mmu-miR-122-5p, mmu-miR-12200-3p, mmu-miR-12200-5p, mmu-miR-12201-3p, mmu-miR-12201-5p, mmu-miR-12202-3p, mmu-miR-12202-5p, mmu-miR-12203-3p, mmu-miR-12203-5p, mmu-miR-12204-5p, mmu-miR-12205-3p, mmu-miR-12205-5p, mmu-miR-12206-5p, mmu-miR-1224-3p, mmu-miR-1224-5p, mmu-miR-122b-3p, mmu-miR-122b-5p, mmu-miR-1231-3p, mmu-miR-1231-5p, mmu-miR-124-3p, mmu-miR-124-5p, mmu-miR-1247-3p, mmu-miR-1247-5p, mmu-miR-1249-3p, mmu-miR-1249-5p, mmu-miR-124b-3p, mmu-miR-1251-3p, mmu-miR-1251-5p, mmu-miR-1258-3p, mmu-miR-1258-5p, mmu-miR-125a-3p, mmu-miR-125a-5p, mmu-miR-125b-1-3p, mmu-miR-125b-2-3p, mmu-miR-125b-5p, mmu-miR-1264-3p, mmu-miR-1264-5p, mmu-miR-126a-3p, mmu-miR-126a-5p, mmu-miR-126b-3p, mmu-miR-126b-5p, mmu-miR-127-3p, mmu-miR-127-5p, mmu-miR-1271-3p, mmu-miR-1271-5p, mmu-miR-128-1-5p, mmu-miR-128-2-5p, mmu-miR-128-3p, mmu-miR-129-1-3p, mmu-miR-129-2-3p, mmu-miR-129-5p, mmu-miR-1291, mmu-miR-1298-3p, mmu-miR-1298-5p, mmu-miR-129b-3p, mmu-miR-129b-5p, mmu-miR-1306-3p, mmu-miR-1306-5p, mmu-miR-130a-3p, mmu-miR-130a-5p, mmu-miR-130b-3p, mmu-miR-130b-5p, mmu-miR-130c, mmu-miR-132-3p, mmu-miR-132-5p, mmu-miR-133a-3p, mmu-miR-133a-5p, mmu-miR-133b-3p, mmu-miR-133b-5p, mmu-miR-133c, mmu-miR-134-3p, mmu-miR-134-5p, mmu-miR-135a-1-3p, mmu-miR-135a-2-3p, mmu-miR-135a-5p, mmu-miR-135b-3p, mmu-miR-135b-5p, mmu-miR-136-3p, mmu-miR-136-5p, mmu-miR-137-3p, mmu-miR-137-5p, mmu-miR-138-1-3p, mmu-miR-138-2-3p, mmu-miR-138-5p, mmu-miR-139-3p, mmu-miR-139-5p, mmu-miR-140-3p, mmu-miR-140-5p, mmu-miR-141-3p, mmu-miR-141-5p, mmu-miR-142a-3p, mmu-miR-142a-5p, mmu-miR-142b, mmu-miR-143-3p, mmu-miR-143-5p, mmu-miR-144-3p, mmu-miR-144-5p, mmu-miR-145a-3p, mmu-miR-145a-5p, mmu-miR-145b, mmu-miR-146a-3p, mmu-miR-146a-5p, mmu-miR-146b-3p, mmu-miR-146b-5p, mmu-miR-147-3p, mmu-miR-147-5p, mmu-miR-148a-3p, mmu-miR-148a-5p, mmu-miR-148b-3p, mmu-miR-148b-5p, mmu-miR-149-3p, mmu-miR-149-5p, mmu-miR-150-3p, mmu-miR-150-5p, mmu-miR-151-3p, mmu-miR-151-5p, mmu-miR-152-3p, mmu-miR-152-5p, mmu-miR-153-3p, mmu-miR-153-5p, mmu-miR-154-3p, mmu-miR-154-5p, mmu-miR-155-3p, mmu-miR-155-5p, mmu-miR-15a-3p, mmu-miR-15a-5p, mmu-miR-15b-3p, mmu-miR-15b-5p, mmu-miR-16-1-3p, mmu-miR-16-2-3p, mmu-miR-16-5p, mmu-miR-1668, mmu-miR-17-3p, mmu-miR-17-5p, mmu-miR-181a-1-3p, mmu-miR-181a-2-3p, mmu-miR-181a-5p, mmu-miR-181b-1-3p, mmu-miR-181b-2-3p, mmu-miR-181b-5p, mmu-miR-181c-3p, mmu-miR-181c-5p, mmu-miR-181d-3p, mmu-miR-181d-5p, mmu-miR-182-3p, mmu-miR-182-5p, mmu-miR-183-3p, mmu-miR-183-5p, mmu-miR-1839-3p, mmu-miR-1839-5p, mmu-miR-184-3p, mmu-miR-184-5p, mmu-miR-1843a-3p, mmu-miR-1843a-5p, mmu-miR-1843b-3p, mmu-miR-1843b-5p, mmu-miR-185-3p, mmu-miR-185-5p, mmu-miR-186-3p, mmu-miR-186-5p, mmu-miR-187-3p, mmu-miR-187-5p, mmu-miR-188-3p, mmu-miR-188-5p, mmu-miR-1892, mmu-miR-1893, mmu-miR-1894-3p, mmu-miR-1894-5p, mmu-miR-1895, mmu-miR-1896, mmu-miR-1897-3p, mmu-miR-1897-5p, mmu-miR-1898, mmu-miR-1899, mmu-miR-18a-3p, mmu-miR-18a-5p, mmu-miR-18b-3p, mmu-miR-18b-5p, mmu-miR-1900, mmu-miR-1901, mmu-miR-1902, mmu-miR-1903, mmu-miR-1904, mmu-miR-1905, mmu-miR-1906, mmu-miR-1907, mmu-miR-190a-3p, mmu-miR-190a-5p, mmu-miR-190b-3p, mmu-miR-190b-5p, mmu-miR-191-3p, mmu-miR-191-5p, mmu-miR-1911-3p, mmu-miR-1911-5p, mmu-miR-1912-3p, mmu-miR-1912-5p, mmu-miR-192-3p, mmu-miR-192-5p, mmu-miR-1927, mmu-miR-1928, mmu-miR-1929-3p, mmu-miR-1929-5p, mmu-miR-1930-3p, mmu-miR-1930-5p, mmu-miR-1931, mmu-miR-1932, mmu-miR-1933-3p, mmu-miR-1933-5p, mmu-miR-1934-3p, mmu-miR-1934-5p, mmu-miR-1936, mmu-miR-1938, mmu-miR-193a-3p, mmu-miR-193a-5p, mmu-miR-193b-3p, mmu-miR-193b-5p, mmu-miR-194-1-3p, mmu-miR-194-2-3p, mmu-miR-194-5p, mmu-miR-1941-3p, mmu-miR-1941-5p, mmu-miR-1942, mmu-miR-1943-3p, mmu-miR-1943-5p, mmu-miR-1945, mmu-miR-1946a, mmu-miR-1946b, mmu-miR-1947-3p, mmu-miR-1947-5p, mmu-miR-1948-3p, mmu-miR-1948-5p, mmu-miR-1949, mmu-miR-1950, mmu-miR-1951, mmu-miR-1952, mmu-miR-1953, mmu-miR-1954, mmu-miR-1955-3p, mmu-miR-1955-5p, mmu-miR-1956, mmu-miR-1958, mmu-miR-195a-3p, mmu-miR-195a-5p, mmu-miR-195b, mmu-miR-1960, mmu-miR-1961, mmu-miR-1962, mmu-miR-1963, mmu-miR-1964-3p, mmu-miR-1964-5p, mmu-miR-1966-3p, mmu-miR-1966-5p, mmu-miR-1967, mmu-miR-1968-3p, mmu-miR-1968-5p, mmu-miR-1969, mmu-miR-196a-1-3p, mmu-miR-196a-2-3p, mmu-miR-196a-5p, mmu-miR-196b-3p, mmu-miR-196b-5p, mmu-miR-1970, mmu-miR-1970b-5p, mmu-miR-1970c-3p, mmu-miR-1970c-5p, mmu-miR-1971, mmu-miR-1981-3p, mmu-miR-1981-5p, mmu-miR-1982-3p, mmu-miR-1982-5p, mmu-miR-1983, mmu-miR-199a-3p, mmu-miR-199a-5p, mmu-miR-199b-3p, mmu-miR-199b-5p, mmu-miR-19a-3p, mmu-miR-19a-5p, mmu-miR-19b-1-5p, mmu-miR-19b-2-5p, mmu-miR-19b-3p, mmu-miR-1a-1-5p, mmu-miR-1a-2-5p, mmu-miR-1a-3p, mmu-miR-1b-3p, mmu-miR-1b-5p, mmu-miR-200a-3p, mmu-miR-200a-5p, mmu-miR-200b-3p, mmu-miR-200b-5p, mmu-miR-200c-3p, mmu-miR-200c-5p, mmu-miR-201-3p, mmu-miR-201-5p, mmu-miR-202-3p, mmu-miR-202-5p, mmu-miR-203-3p, mmu-miR-203-5p, mmu-miR-203b-3p, mmu-miR-203b-5p, mmu-miR-204-3p, mmu-miR-204-5p, mmu-miR-205-3p, mmu-miR-205-5p, mmu-miR-206-3p, mmu-miR-206-5p, mmu-miR-207, mmu-miR-208a-3p, mmu-miR-208a-5p, mmu-miR-208b-3p, mmu-miR-208b-5p, mmu-miR-20a-3p, mmu-miR-20a-5p, mmu-miR-20b-3p, mmu-miR-20b-5p, mmu-miR-210-3p, mmu-miR-210-5p, mmu-miR-211-3p, mmu-miR-211-5p, mmu-miR-212-3p, mmu-miR-212-5p, mmu-miR-2136, mmu-miR-2137, mmu-miR-2139, mmu-miR-214-3p, mmu-miR-214-5p, mmu-miR-215-3p, mmu-miR-215-5p, mmu-miR-216a-3p, mmu-miR-216a-5p, mmu-miR-216b-3p, mmu-miR-216b-5p, mmu-miR-216c-3p, mmu-miR-216c-5p, mmu-miR-217-3p, mmu-miR-217-5p, mmu-miR-218-1-3p, mmu-miR-218-2-3p, mmu-miR-218-5p, mmu-miR-2183, mmu-miR-219a-1-3p, mmu-miR-219a-2-3p, mmu-miR-219a-5p, mmu-miR-219b-3p, mmu-miR-219b-5p, mmu-miR-219c-3p, mmu-miR-219c-5p, mmu-miR-21a-3p, mmu-miR-21a-5p, mmu-miR-21b, mmu-miR-21c, mmu-miR-22-3p, mmu-miR-22-5p, mmu-miR-221-3p, mmu-miR-221-5p, mmu-miR-222-3p, mmu-miR-222-5p, mmu-miR-223-3p, mmu-miR-223-5p, mmu-miR-224-3p, mmu-miR-224-5p, mmu-miR-23a-3p, mmu-miR-23a-5p, mmu-miR-23b-3p, mmu-miR-23b-5p, mmu-miR-24-1-5p, mmu-miR-24-2-5p, mmu-miR-24-3p, mmu-miR-25-3p, mmu-miR-25-5p, mmu-miR-26a-1-3p, mmu-miR-26a-2-3p, mmu-miR-26a-5p, mmu-miR-26b-3p, mmu-miR-26b-5p, mmu-miR-27a-3p, mmu-miR-27a-5p, mmu-miR-27b-3p, mmu-miR-27b-5p, mmu-miR-2861, mmu-miR-28a-3p, mmu-miR-28a-5p, mmu-miR-28b, mmu-miR-28c, mmu-miR-290a-3p, mmu-miR-290a-5p, mmu-miR-290b-3p, mmu-miR-290b-5p, mmu-miR-291a-3p, mmu-miR-291a-5p, mmu-miR-291b-3p, mmu-miR-291b-5p, mmu-miR-292a-3p, mmu-miR-292a-5p, mmu-miR-292b-3p, mmu-miR-292b-5p, mmu-miR-293-3p, mmu-miR-293-5p, mmu-miR-294-3p, mmu-miR-294-5p, mmu-miR-295-3p, mmu-miR-295-5p, mmu-miR-296-3p, mmu-miR-296-5p, mmu-miR-297a-3p, mmu-miR-297a-5p, mmu-miR-297b-3p, mmu-miR-297b-5p, mmu-miR-297c-3p, mmu-miR-297c-5p, mmu-miR-298-3p, mmu-miR-298-5p, mmu-miR-299a-3p, mmu-miR-299a-5p, mmu-miR-299b-3p, mmu-miR-299b-5p, mmu-miR-29a-3p, mmu-miR-29a-5p, mmu-miR-29b-1-5p, mmu-miR-29b-2-5p, mmu-miR-29b-3p, mmu-miR-29c-3p, mmu-miR-29c-5p, mmu-miR-300-3p, mmu-miR-300-5p, mmu-miR-301a-3p, mmu-miR-301a-5p, mmu-miR-301b-3p, mmu-miR-301b-5p, mmu-miR-302a-3p, mmu-miR-302a-5p, mmu-miR-302b-3p, mmu-miR-302b-5p, mmu-miR-302c-3p, mmu-miR-302c-5p, mmu-miR-302d-3p, mmu-miR-302d-5p, mmu-miR-3057-3p, mmu-miR-3057-5p, mmu-miR-3058-3p, mmu-miR-3058-5p, mmu-miR-3059-3p, mmu-miR-3059-5p, mmu-miR-3060-3p, mmu-miR-3060-5p, mmu-miR-3061-3p, mmu-miR-3061-5p, mmu-miR-3062-3p, mmu-miR-3062-5p, mmu-miR-3063-3p, mmu-miR-3063-5p, mmu-miR-3064-3p, mmu-miR-3064-5p, mmu-miR-3065-3p, mmu-miR-3065-5p, mmu-miR-3066-3p, mmu-miR-3066-5p, mmu-miR-3067-3p, mmu-miR-3067-5p, mmu-miR-3068-3p, mmu-miR-3068-5p, mmu-miR-3069-3p, mmu-miR-3069-5p, mmu-miR-3070-2-3p, mmu-miR-3070-3p, mmu-miR-3070-5p, mmu-miR-3071-3p, mmu-miR-3071-5p, mmu-miR-3072-3p, mmu-miR-3072-5p, mmu-miR-3073a-3p, mmu-miR-3073a-5p, mmu-miR-3073b-3p, mmu-miR-3073b-5p, mmu-miR-3074-1-3p, mmu-miR-3074-2-3p, mmu-miR-3074-5p, mmu-miR-3075-3p, mmu-miR-3075-5p, mmu-miR-3076-3p, mmu-miR-3076-5p, mmu-miR-3077-3p, mmu-miR-3077-5p, mmu-miR-3078-3p, mmu-miR-3078-5p, mmu-miR-3079-3p, mmu-miR-3079-5p, mmu-miR-3080-3p, mmu-miR-3080-5p, mmu-miR-3081-3p, mmu-miR-3081-5p, mmu-miR-3082-3p, mmu-miR-3082-5p, mmu-miR-3083-3p, mmu-miR-3083-5p, mmu-miR-3083b-3p, mmu-miR-3083b-5p, mmu-miR-3084-3p, mmu-miR-3084-5p, mmu-miR-3085-3p, mmu-miR-3085-5p, mmu-miR-3086-3p, mmu-miR-3086-5p, mmu-miR-3087-3p, mmu-miR-3087-5p, mmu-miR-3088-3p, mmu-miR-3088-5p, mmu-miR-3089-3p, mmu-miR-3089-5p, mmu-miR-3090-3p, mmu-miR-3090-5p, mmu-miR-3091-3p, mmu-miR-3091-5p, mmu-miR-3092-3p, mmu-miR-3092-5p, mmu-miR-3093-3p, mmu-miR-3093-5p, mmu-miR-3094-3p, mmu-miR-3094-5p, mmu-miR-3095-3p, mmu-miR-3095-5p, mmu-miR-3097-3p, mmu-miR-3097-5p, mmu-miR-3098-3p, mmu-miR-3098-5p, mmu-miR-3099-3p, mmu-miR-3099-5p, mmu-miR-30a-3p, mmu-miR-30a-5p, mmu-miR-30b-3p, mmu-miR-30b-5p, mmu-miR-30c-1-3p, mmu-miR-30c-2-3p, mmu-miR-30c-5p, mmu-miR-30d-3p, mmu-miR-30d-5p, mmu-miR-30e-3p, mmu-miR-30e-5p, mmu-miR-30f, mmu-miR-31-3p, mmu-miR-31-5p, mmu-miR-3100-3p, mmu-miR-3100-5p, mmu-miR-3101-3p, mmu-miR-3101-5p, mmu-miR-3102-3p, mmu-miR-3102-3p.2-3p, mmu-miR-3102-5p, mmu-miR-3102-5p.2-5p, mmu-miR-3103-3p, mmu-miR-3103-5p, mmu-miR-3104-3p, mmu-miR-3104-5p, mmu-miR-3105-3p, mmu-miR-3105-5p, mmu-miR-3106-3p, mmu-miR-3106-5p, mmu-miR-3108-3p, mmu-miR-3108-5p, mmu-miR-3109-3p, mmu-miR-3109-5p, mmu-miR-3110-3p, mmu-miR-3110-5p, mmu-miR-3112-3p, mmu-miR-3112-5p, mmu-miR-3113-3p, mmu-miR-3113-5p, mmu-miR-3154, mmu-miR-32-3p, mmu-miR-32-5p, mmu-miR-320-3p, mmu-miR-320-5p, mmu-miR-322-3p, mmu-miR-322-5p, mmu-miR-323-3p, mmu-miR-323-5p, mmu-miR-324-3p, mmu-miR-324-5p, mmu-miR-325-3p, mmu-miR-325-5p, mmu-miR-326-3p, mmu-miR-326-5p, mmu-miR-327, mmu-miR-328-3p, mmu-miR-328-5p, mmu-miR-329-3p, mmu-miR-329-5p, mmu-miR-33-3p, mmu-miR-33-5p, mmu-miR-330-3p, mmu-miR-330-5p, mmu-miR-331-3p, mmu-miR-331-5p, mmu-miR-335-3p, mmu-miR-335-5p, mmu-miR-337-3p, mmu-miR-337-5p, mmu-miR-338-3p, mmu-miR-338-5p, mmu-miR-339-3p, mmu-miR-339-5p, mmu-miR-340-3p, mmu-miR-340-5p, mmu-miR-341-3p, mmu-miR-341-5p, mmu-miR-342-3p, mmu-miR-342-5p, mmu-miR-343, mmu-miR-344-3p, mmu-miR-344-5p, mmu-miR-344b-3p, mmu-miR-344b-5p, mmu-miR-344c-3p, mmu-miR-344c-5p, mmu-miR-344d-1-5p, mmu-miR-344d-2-5p, mmu-miR-344d-3-5p, mmu-miR-344d-3p, mmu-miR-344e-3p, mmu-miR-344e-5p, mmu-miR-344f-3p, mmu-miR-344f-5p, mmu-miR-344g-3p, mmu-miR-344g-5p, mmu-miR-344 h-3p, mmu-miR-344 h-5p, mmu-miR-344i, mmu-miR-345-3p, mmu-miR-345-5p, mmu-miR-346-3p, mmu-miR-346-5p, mmu-miR-3470a, mmu-miR-3470b, mmu-miR-3471, mmu-miR-3472, mmu-miR-3473a, mmu-miR-3473b, mmu-miR-3473c, mmu-miR-3473d, mmu-miR-3473e, mmu-miR-3473f, mmu-miR-3473g, mmu-miR-3473 h-5p, mmu-miR-3474, mmu-miR-3475-3p, mmu-miR-3475-5p, mmu-miR-34a-3p, mmu-miR-34a-5p, mmu-miR-34b-3p, mmu-miR-34b-5p, mmu-miR-34c-3p, mmu-miR-34c-5p, mmu-miR-350-3p, mmu-miR-350-5p, mmu-miR-351-3p, mmu-miR-351-5p, mmu-miR-3535, mmu-miR-3544-3p, mmu-miR-3544-5p, mmu-miR-3547-3p, mmu-miR-3547-5p, mmu-miR-3552, mmu-miR-3569-3p, mmu-miR-3569-5p, mmu-miR-3572-3p, mmu-miR-3572-5p, mmu-miR-361-3p, mmu-miR-361-5p, mmu-miR-3618-3p, mmu-miR-3618-5p, mmu-miR-362-3p, mmu-miR-362-5p, mmu-miR-3620-3p, mmu-miR-3620-5p, mmu-miR-363-3p, mmu-miR-363-5p, mmu-miR-365-1-5p, mmu-miR-365-2-5p, mmu-miR-365-3p, mmu-miR-367-3p, mmu-miR-367-5p, mmu-miR-369-3p, mmu-miR-369-5p, mmu-miR-370-3p, mmu-miR-370-5p, mmu-miR-374b-3p, mmu-miR-374b-5p, mmu-miR-374c-3p, mmu-miR-374c-5p, mmu-miR-375-3p, mmu-miR-375-5p, mmu-miR-376a-3p, mmu-miR-376a-5p, mmu-miR-376b-3p, mmu-miR-376b-5p, mmu-miR-376c-3p, mmu-miR-376c-5p, mmu-miR-377-3p, mmu-miR-377-5p, mmu-miR-378a-3p, mmu-miR-378a-5p, mmu-miR-378b, mmu-miR-378c, mmu-miR-378d, mmu-miR-379-3p, mmu-miR-379-5p, mmu-miR-380-3p, mmu-miR-380-5p, mmu-miR-381-3p, mmu-miR-381-5p, mmu-miR-382-3p, mmu-miR-382-5p, mmu-miR-383-3p, mmu-miR-383-5p, mmu-miR-384-3p, mmu-miR-384-5p, mmu-miR-3960, mmu-miR-3961, mmu-miR-3962, mmu-miR-3963, mmu-miR-3964, mmu-miR-3965, mmu-miR-3966, mmu-miR-3967, mmu-miR-3968, mmu-miR-3969, mmu-miR-3970, mmu-miR-3971, mmu-miR-409-3p, mmu-miR-409-5p, mmu-miR-410-3p, mmu-miR-410-5p, mmu-miR-411-3p, mmu-miR-411-5p, mmu-miR-412-3p, mmu-miR-412-5p, mmu-miR-421-3p, mmu-miR-421-5p, mmu-miR-423-3p, mmu-miR-423-5p, mmu-miR-425-3p, mmu-miR-425-5p, mmu-miR-429-3p, mmu-miR-429-5p, mmu-miR-431-3p, mmu-miR-431-5p, mmu-miR-432, mmu-miR-433-3p, mmu-miR-433-5p, mmu-miR-434-3p, mmu-miR-434-5p, mmu-miR-448-3p, mmu-miR-448-5p, mmu-miR-449a-3p, mmu-miR-449a-5p, mmu-miR-449b, mmu-miR-449c-3p, mmu-miR-449c-5p, mmu-miR-450a-1-3p, mmu-miR-450a-2-3p, mmu-miR-450a-5p, mmu-miR-450b-3p, mmu-miR-450b-5p, mmu-miR-451a, mmu-miR-451b, mmu-miR-452-3p, mmu-miR-452-5p, mmu-miR-453, mmu-miR-455-3p, mmu-miR-455-5p, mmu-miR-463-3p, mmu-miR-463-5p, mmu-miR-465a-3p, mmu-miR-465a-5p, mmu-miR-465b-3p, mmu-miR-465b-5p, mmu-miR-465c-3p, mmu-miR-465c-5p, mmu-miR-465d-3p, mmu-miR-465d-5p, mmu-miR-466a-3p, mmu-miR-466a-5p, mmu-miR-466b-3p, mmu-miR-466b-5p, mmu-miR-466c-3p, mmu-miR-466c-5p, mmu-miR-466d-3p, mmu-miR-466d-5p, mmu-miR-466e-3p, mmu-miR-466e-5p, mmu-miR-466f, mmu-miR-466f-3p, mmu-miR-466f-5p, mmu-miR-466g, mmu-miR-466 h-3p, mmu-miR-466 h-5p, mmu-miR-466i-3p, mmu-miR-466i-5p, mmu-miR-466j, mmu-miR-466k, mmu-miR-4661-3p, mmu-miR-4661-5p, mmu-miR-466m-3p, mmu-miR-466m-5p, mmu-miR-466n-3p, mmu-miR-466n-5p, mmu-miR-4660-3p, mmu-miR-4660-5p, mmu-miR-466p-3p, mmu-miR-466p-5p, mmu-miR-466q, mmu-miR-467a-3p, mmu-miR-467a-5p, mmu-miR-467b-3p, mmu-miR-467b-5p, mmu-miR-467c-3p, mmu-miR-467c-5p, mmu-miR-467d-3p, mmu-miR-467d-5p, mmu-miR-467e-3p, mmu-miR-467e-5p, mmu-miR-467f, mmu-miR-467g, mmu-miR-467h, mmu-miR-468-3p, mmu-miR-468-5p, mmu-miR-470-3p, mmu-miR-470-5p, mmu-miR-471-3p, mmu-miR-471-5p, mmu-miR-483-3p, mmu-miR-483-5p, mmu-miR-484, mmu-miR-485-3p, mmu-miR-485-5p, mmu-miR-486a-3p, mmu-miR-486a-5p, mmu-miR-486b-3p, mmu-miR-486b-5p, mmu-miR-487b-3p, mmu-miR-487b-5p, mmu-miR-488-3p, mmu-miR-488-5p, mmu-miR-489-3p, mmu-miR-489-5p, mmu-miR-490-3p, mmu-miR-490-5p, mmu-miR-491-3p, mmu-miR-491-5p, mmu-miR-493-3p, mmu-miR-493-5p, mmu-miR-494-3p, mmu-miR-494-5p, mmu-miR-495-3p, mmu-miR-495-5p, mmu-miR-496a-3p, mmu-miR-496a-5p, mmu-miR-496b, mmu-miR-497a-3p, mmu-miR-497a-5p, mmu-miR-497b, mmu-miR-499-3p, mmu-miR-499-5p, mmu-miR-500-3p, mmu-miR-500-5p, mmu-miR-501-3p, mmu-miR-501-5p, mmu-miR-503-3p, mmu-miR-503-5p, mmu-miR-504-3p, mmu-miR-504-5p, mmu-miR-5046, mmu-miR-505-3p, mmu-miR-505-5p, mmu-miR-509-3p, mmu-miR-509-5p, mmu-miR-5098, mmu-miR-5099, mmu-miR-5100, mmu-miR-5101, mmu-miR-5103, mmu-miR-5104, mmu-miR-5104b-5p, mmu-miR-5106, mmu-miR-5107-3p, mmu-miR-5107-5p, mmu-miR-5108, mmu-miR-511-3p, mmu-miR-511-5p, mmu-miR-5110, mmu-miR-5112, mmu-miR-5113, mmu-miR-5114, mmu-miR-5116, mmu-miR-5118, mmu-miR-5119, mmu-miR-5120, mmu-miR-5121, mmu-miR-5122, mmu-miR-5123, mmu-miR-5124a, mmu-miR-5124b, mmu-miR-5125, mmu-miR-5126, mmu-miR-5127, mmu-miR-5128, mmu-miR-5129-3p, mmu-miR-5129-5p, mmu-miR-5130, mmu-miR-5131, mmu-miR-5132-3p, mmu-miR-5132-5p, mmu-miR-5133, mmu-miR-5134-3p, mmu-miR-5134-5p, mmu-miR-5135, mmu-miR-5136, mmu-miR-532-3p, mmu-miR-532-5p, mmu-miR-539-3p, mmu-miR-539-5p, mmu-miR-540-3p, mmu-miR-540-5p, mmu-miR-541-3p, mmu-miR-541-5p, mmu-miR-542-3p, mmu-miR-542-5p, mmu-miR-543-3p, mmu-miR-543-5p, mmu-miR-544-3p, mmu-miR-544-5p, mmu-miR-546, mmu-miR-547-3p, mmu-miR-547-5p, mmu-miR-551b-3p, mmu-miR-551b-5p, mmu-miR-5615-3p, mmu-miR-5615-5p, mmu-miR-5616-3p, mmu-miR-5616-5p, mmu-miR-5617-3p, mmu-miR-5617-5p, mmu-miR-5618-3p, mmu-miR-5618-5p, mmu-miR-5619-3p, mmu-miR-5619-5p, mmu-miR-5620-3p, mmu-miR-5620-5p, mmu-miR-5621-3p, mmu-miR-5621-5p, mmu-miR-5622-3p, mmu-miR-5622-5p, mmu-miR-5623-3p, mmu-miR-5623-5p, mmu-miR-5624-3p, mmu-miR-5624-5p, mmu-miR-5625-3p, mmu-miR-5625-5p, mmu-miR-5626-3p, mmu-miR-5626-5p, mmu-miR-5627-3p, mmu-miR-5627-5p, mmu-miR-568, mmu-miR-5709-3p, mmu-miR-5709-5p, mmu-miR-5710, mmu-miR-574-3p, mmu-miR-574-5p, mmu-miR-582-3p, mmu-miR-582-5p, mmu-miR-590-3p, mmu-miR-590-5p, mmu-miR-592-3p, mmu-miR-592-5p, mmu-miR-598-3p, mmu-miR-598-5p, mmu-miR-599, mmu-miR-615-3p, mmu-miR-615-5p, mmu-miR-6236, mmu-miR-6237, mmu-miR-6238, mmu-miR-6239, mmu-miR-6240, mmu-miR-6241, mmu-miR-6244, mmu-miR-6335, mmu-miR-6336, mmu-miR-6337, mmu-miR-6338, mmu-miR-6339, mmu-miR-6340, mmu-miR-6341, mmu-miR-6342, mmu-miR-6343, mmu-miR-6344, mmu-miR-6345, mmu-miR-6346, mmu-miR-6347, mmu-miR-6348, mmu-miR-6349, mmu-miR-6350, mmu-miR-6351, mmu-miR-6352, mmu-miR-6353, mmu-miR-6354, mmu-miR-6355, mmu-miR-6356, mmu-miR-6357, mmu-miR-6358, mmu-miR-6359, mmu-miR-6360, mmu-miR-6361, mmu-miR-6362, mmu-miR-6363, mmu-miR-6364, mmu-miR-6365, mmu-miR-6366, mmu-miR-6367, mmu-miR-6368, mmu-miR-6369, mmu-miR-6370, mmu-miR-6371, mmu-miR-6372, mmu-miR-6373, mmu-miR-6374, mmu-miR-6375, mmu-miR-6376, mmu-miR-6377, mmu-miR-6378, mmu-miR-6379, mmu-miR-6380, mmu-miR-6381, mmu-miR-6382, mmu-miR-6383, mmu-miR-6384, mmu-miR-6385, mmu-miR-6386, mmu-miR-6387, mmu-miR-6388, mmu-miR-6389, mmu-miR-6390, mmu-miR-6391, mmu-miR-6392-3p, mmu-miR-6392-5p, mmu-miR-6393, mmu-miR-6394, mmu-miR-6395, mmu-miR-6396, mmu-miR-6397, mmu-miR-6398, mmu-miR-6399, mmu-miR-6400, mmu-miR-6401, mmu-miR-6402, mmu-miR-6403, mmu-miR-6404, mmu-miR-6405, mmu-miR-6406, mmu-miR-6407, mmu-miR-6408, mmu-miR-6409, mmu-miR-6410, mmu-miR-6411, mmu-miR-6412, mmu-miR-6413, mmu-miR-6414, mmu-miR-6415, mmu-miR-6416-3p, mmu-miR-6416-5p, mmu-miR-6417, mmu-miR-6418-3p, mmu-miR-6418-5p, mmu-miR-6419, mmu-miR-6420, mmu-miR-6481, mmu-miR-6516-3p, mmu-miR-6516-5p, mmu-miR-652-3p, mmu-miR-652-5p, mmu-miR-653-3p, mmu-miR-653-5p, mmu-miR-6537-3p, mmu-miR-6537-5p, mmu-miR-6538, mmu-miR-6539, mmu-miR-654-3p, mmu-miR-654-5p, mmu-miR-6540-3p, mmu-miR-6540-5p, mmu-miR-6541, mmu-miR-6546-3p, mmu-miR-6546-5p, mmu-miR-664-3p, mmu-miR-664-5p, mmu-miR-665-3p, mmu-miR-665-5p, mmu-miR-666-3p, mmu-miR-666-5p, mmu-miR-667-3p, mmu-miR-667-5p, mmu-miR-668-3p, mmu-miR-668-5p, mmu-miR-669a-3-3p, mmu-miR-669a-3p, mmu-miR-669a-5p, mmu-miR-669b-3p, mmu-miR-669b-5p, mmu-miR-669c-3p, mmu-miR-669c-5p, mmu-miR-669d-2-3p, mmu-miR-669d-3p, mmu-miR-669d-5p, mmu-miR-669e-3p, mmu-miR-669e-5p, mmu-miR-669f-3p, mmu-miR-669f-5p, mmu-miR-669g, mmu-miR-669 h-3p, mmu-miR-669 h-5p, mmu-miR-669i, mmu-miR-669j, mmu-miR-669k-3p, mmu-miR-669k-5p, mmu-miR-6691-3p, mmu-miR-6691-5p, mmu-miR-669m-3p, mmu-miR-669m-5p, mmu-miR-669n, mmu-miR-6690-3p, mmu-miR-6690-5p, mmu-miR-669p-3p, mmu-miR-669p-5p, mmu-miR-670-3p, mmu-miR-670-5p, mmu-miR-671-3p, mmu-miR-671-5p, mmu-miR-6715-3p, mmu-miR-6715-5p, mmu-miR-672-3p, mmu-miR-672-5p, mmu-miR-673-3p, mmu-miR-673-5p, mmu-miR-674-3p, mmu-miR-674-5p, mmu-miR-675-3p, mmu-miR-675-5p, mmu-miR-676-3p, mmu-miR-676-5p, mmu-miR-6769b-3p, mmu-miR-6769b-5p, mmu-miR-677-3p, mmu-miR-677-5p, mmu-miR-678, mmu-miR-679-3p, mmu-miR-679-5p, mmu-miR-680, mmu-miR-681, mmu-miR-682, mmu-miR-683, mmu-miR-684, mmu-miR-686, mmu-miR-687, mmu-miR-688, mmu-miR-6896-3p, mmu-miR-6896-5p, mmu-miR-6897-3p, mmu-miR-6897-5p, mmu-miR-6898-3p, mmu-miR-6898-5p, mmu-miR-6899-3p, mmu-miR-6899-5p, mmu-miR-690, mmu-miR-6900-3p, mmu-miR-6900-5p, mmu-miR-6901-3p, mmu-miR-6901-5p, mmu-miR-6902-3p, mmu-miR-6902-5p, mmu-miR-6903-3p, mmu-miR-6903-5p, mmu-miR-6904-3p, mmu-miR-6904-5p, mmu-miR-6905-3p, mmu-miR-6905-5p, mmu-miR-6906-3p, mmu-miR-6906-5p, mmu-miR-6907-3p, mmu-miR-6907-5p, mmu-miR-6908-3p, mmu-miR-6908-5p, mmu-miR-6909-3p, mmu-miR-6909-5p, mmu-miR-691, mmu-miR-6910-3p, mmu-miR-6910-5p, mmu-miR-6911-3p, mmu-miR-6911-5p, mmu-miR-6912-3p, mmu-miR-6912-5p, mmu-miR-6913-3p, mmu-miR-6913-5p, mmu-miR-6914-3p, mmu-miR-6914-5p, mmu-miR-6915-3p, mmu-miR-6915-5p, mmu-miR-6916-3p, mmu-miR-6916-5p, mmu-miR-6917-3p, mmu-miR-6917-5p, mmu-miR-6918-3p, mmu-miR-6918-5p, mmu-miR-6919-3p, mmu-miR-6919-5p, mmu-miR-692, mmu-miR-6920-3p, mmu-miR-6920-5p, mmu-miR-6921-3p, mmu-miR-6921-5p, mmu-miR-6922-3p, mmu-miR-6922-5p, mmu-miR-6923-3p, mmu-miR-6923-5p, mmu-miR-6924-3p, mmu-miR-6924-5p, mmu-miR-6925-3p, mmu-miR-6925-5p, mmu-miR-6926-3p, mmu-miR-6926-5p, mmu-miR-6927-3p, mmu-miR-6927-5p, mmu-miR-6928-3p, mmu-miR-6928-5p, mmu-miR-6929-3p, mmu-miR-6929-5p, mmu-miR-693-3p, mmu-miR-693-5p, mmu-miR-6930-3p, mmu-miR-6930-5p, mmu-miR-6931-3p, mmu-miR-6931-5p, mmu-miR-6932-3p, mmu-miR-6932-5p, mmu-miR-6933-3p, mmu-miR-6933-5p, mmu-miR-6934-3p, mmu-miR-6934-5p, mmu-miR-6935-3p, mmu-miR-6935-5p, mmu-miR-6936-3p, mmu-miR-6936-5p, mmu-miR-6937-3p, mmu-miR-6937-5p, mmu-miR-6938-3p, mmu-miR-6938-5p, mmu-miR-6939-3p, mmu-miR-6939-5p, mmu-miR-694, mmu-miR-6940-3p, mmu-miR-6940-5p, mmu-miR-6941-3p, mmu-miR-6941-5p, mmu-miR-6942-3p, mmu-miR-6942-5p, mmu-miR-6943-3p, mmu-miR-6943-5p, mmu-miR-6944-3p, mmu-miR-6944-5p, mmu-miR-6945-3p, mmu-miR-6945-5p, mmu-miR-6946-3p, mmu-miR-6946-5p, mmu-miR-6947-3p, mmu-miR-6947-5p, mmu-miR-6948-3p, mmu-miR-6948-5p, mmu-miR-6949-3p, mmu-miR-6949-5p, mmu-miR-695, mmu-miR-6950-3p, mmu-miR-6950-5p, mmu-miR-6951-3p, mmu-miR-6951-5p, mmu-miR-6952-3p, mmu-miR-6952-5p, mmu-miR-6953-3p, mmu-miR-6953-5p, mmu-miR-6954-3p, mmu-miR-6954-5p, mmu-miR-6955-3p, mmu-miR-6955-5p, mmu-miR-6956-3p, mmu-miR-6956-5p, mmu-miR-6957-3p, mmu-miR-6957-5p, mmu-miR-6958-3p, mmu-miR-6958-5p, mmu-miR-6959-3p, mmu-miR-6959-5p, mmu-miR-696, mmu-miR-6960-3p, mmu-miR-6960-5p, mmu-miR-6961-3p, mmu-miR-6961-5p, mmu-miR-6962-3p, mmu-miR-6962-5p, mmu-miR-6963-3p, mmu-miR-6963-5p, mmu-miR-6964-3p, mmu-miR-6964-5p, mmu-miR-6965-3p, mmu-miR-6965-5p, mmu-miR-6966-3p, mmu-miR-6966-5p, mmu-miR-6967-3p, mmu-miR-6967-5p, mmu-miR-6968-3p, mmu-miR-6968-5p, mmu-miR-6969-3p, mmu-miR-6969-5p, mmu-miR-697, mmu-miR-6970-3p, mmu-miR-6970-5p, mmu-miR-6971-3p, mmu-miR-6971-5p, mmu-miR-6972-3p, mmu-miR-6972-5p, mmu-miR-6973a-3p, mmu-miR-6973a-5p, mmu-miR-6973b-3p, mmu-miR-6973b-5p, mmu-miR-6974-3p, mmu-miR-6974-5p, mmu-miR-6975-3p, mmu-miR-6975-5p, mmu-miR-6976-3p, mmu-miR-6976-5p, mmu-miR-6977-3p, mmu-miR-6977-5p, mmu-miR-6978-3p, mmu-miR-6978-5p, mmu-miR-6979-3p, mmu-miR-6979-5p, mmu-miR-698-3p, mmu-miR-698-5p, mmu-miR-6980-3p, mmu-miR-6980-5p, mmu-miR-6981-3p, mmu-miR-6981-5p, mmu-miR-6982-3p, mmu-miR-6982-5p, mmu-miR-6983-3p, mmu-miR-6983-5p, mmu-miR-6984-3p, mmu-miR-6984-5p, mmu-miR-6985-3p, mmu-miR-6985-5p, mmu-miR-6986-3p, mmu-miR-6986-5p, mmu-miR-6987-3p, mmu-miR-6987-5p, mmu-miR-6988-3p, mmu-miR-6988-5p, mmu-miR-6989-3p, mmu-miR-6989-5p, mmu-miR-6990-3p, mmu-miR-6990-5p, mmu-miR-6991-3p, mmu-miR-6991-5p, mmu-miR-6992-3p, mmu-miR-6992-5p, mmu-miR-6993-3p, mmu-miR-6993-5p, mmu-miR-6994-3p, mmu-miR-6994-5p, mmu-miR-6995-3p, mmu-miR-6995-5p, mmu-miR-6996-3p, mmu-miR-6996-5p, mmu-miR-6997-3p, mmu-miR-6997-5p, mmu-miR-6998-3p, mmu-miR-6998-5p, mmu-miR-6999-3p, mmu-miR-6999-5p, mmu-miR-700-3p, mmu-miR-700-5p, mmu-miR-7000-3p, mmu-miR-7000-5p, mmu-miR-7001-3p, mmu-miR-7001-5p, mmu-miR-7002-3p, mmu-miR-7002-5p, mmu-miR-7003-3p, mmu-miR-7003-5p, mmu-miR-7004-3p, mmu-miR-7004-5p, mmu-miR-7005-3p, mmu-miR-7005-5p, mmu-miR-7006-3p, mmu-miR-7006-5p, mmu-miR-7007-3p, mmu-miR-7007-5p, mmu-miR-7008-3p, mmu-miR-7008-5p, mmu-miR-7009-3p, mmu-miR-7009-5p, mmu-miR-701-3p, mmu-miR-701-5p, mmu-miR-7010-3p, mmu-miR-7010-5p, mmu-miR-7011-3p, mmu-miR-7011-5p, mmu-miR-7012-3p, mmu-miR-7012-5p, mmu-miR-7013-3p, mmu-miR-7013-5p, mmu-miR-7014-3p, mmu-miR-7014-5p, mmu-miR-7015-3p, mmu-miR-7015-5p, mmu-miR-7016-3p, mmu-miR-7016-5p, mmu-miR-7017-3p, mmu-miR-7017-5p, mmu-miR-7018-3p, mmu-miR-7018-5p, mmu-miR-7019-3p, mmu-miR-7019-5p, mmu-miR-702-3p, mmu-miR-702-5p, mmu-miR-7020-3p, mmu-miR-7020-5p, mmu-miR-7021-3p, mmu-miR-7021-5p, mmu-miR-7022-3p, mmu-miR-7022-5p, mmu-miR-7023-3p, mmu-miR-7023-5p, mmu-miR-7024-3p, mmu-miR-7024-5p, mmu-miR-7025-3p, mmu-miR-7025-5p, mmu-miR-7026-3p, mmu-miR-7026-5p, mmu-miR-7027-3p, mmu-miR-7027-5p, mmu-miR-7028-3p, mmu-miR-7028-5p, mmu-miR-7029-3p, mmu-miR-7029-5p, mmu-miR-703, mmu-miR-7030-3p, mmu-miR-7030-5p, mmu-miR-7031-3p, mmu-miR-7031-5p, mmu-miR-7032-3p, mmu-miR-7032-5p, mmu-miR-7033-3p, mmu-miR-7033-5p, mmu-miR-7034-3p, mmu-miR-7034-5p, mmu-miR-7035-3p, mmu-miR-7035-5p, mmu-miR-7036a-3p, mmu-miR-7036a-5p, mmu-miR-7036b-3p, mmu-miR-7036b-5p, mmu-miR-7037-3p, mmu-miR-7037-5p, mmu-miR-7038-3p, mmu-miR-7038-5p, mmu-miR-7039-3p, mmu-miR-7039-5p, mmu-miR-704, mmu-miR-7040-3p, mmu-miR-7040-5p, mmu-miR-7041-3p, mmu-miR-7041-5p, mmu-miR-7042-3p, mmu-miR-7042-5p, mmu-miR-7043-3p, mmu-miR-7043-5p, mmu-miR-7044-3p, mmu-miR-7044-5p, mmu-miR-7045-3p, mmu-miR-7045-5p, mmu-miR-7046-3p, mmu-miR-7046-5p, mmu-miR-7047-3p, mmu-miR-7047-5p, mmu-miR-7048-3p, mmu-miR-7048-5p, mmu-miR-7049-3p, mmu-miR-7049-5p, mmu-miR-705, mmu-miR-7050-3p, mmu-miR-7050-5p, mmu-miR-7051-3p, mmu-miR-7051-5p, mmu-miR-7052-3p, mmu-miR-7052-5p, mmu-miR-7053-3p, mmu-miR-7053-5p, mmu-miR-7054-3p, mmu-miR-7054-5p, mmu-miR-7055-3p, mmu-miR-7055-5p, mmu-miR-7056-3p, mmu-miR-7056-5p, mmu-miR-7057-3p, mmu-miR-7057-5p, mmu-miR-7058-3p, mmu-miR-7058-5p, mmu-miR-7059-3p, mmu-miR-7059-5p, mmu-miR-706, mmu-miR-7060-3p, mmu-miR-7060-5p, mmu-miR-7061-3p, mmu-miR-7061-5p, mmu-miR-7062-3p, mmu-miR-7062-5p, mmu-miR-7063-3p, mmu-miR-7063-5p, mmu-miR-7064-3p, mmu-miR-7064-5p, mmu-miR-7065-3p, mmu-miR-7065-5p, mmu-miR-7066-3p, mmu-miR-7066-5p, mmu-miR-7067-3p, mmu-miR-7067-5p, mmu-miR-7068-3p, mmu-miR-7068-5p, mmu-miR-7069-3p, mmu-miR-7069-5p, mmu-miR-707, mmu-miR-7070-3p, mmu-miR-7070-5p, mmu-miR-7071-3p, mmu-miR-7071-5p, mmu-miR-7072-3p, mmu-miR-7072-5p, mmu-miR-7073-3p, mmu-miR-7073-5p, mmu-miR-7074-3p, mmu-miR-7074-5p, mmu-miR-7075-3p, mmu-miR-7075-5p, mmu-miR-7076-3p, mmu-miR-7076-5p, mmu-miR-7077-3p, mmu-miR-7077-5p, mmu-miR-7078-3p, mmu-miR-7078-5p, mmu-miR-7079-3p, mmu-miR-7079-5p, mmu-miR-708-3p, mmu-miR-708-5p, mmu-miR-7080-3p, mmu-miR-7080-5p, mmu-miR-7081-3p, mmu-miR-7081-5p, mmu-miR-7082-3p, mmu-miR-7082-5p, mmu-miR-7083-3p, mmu-miR-7083-5p, mmu-miR-7084-3p, mmu-miR-7084-5p, mmu-miR-7085-3p, mmu-miR-7085-5p, mmu-miR-7086-3p, mmu-miR-7086-5p, mmu-miR-7087-3p, mmu-miR-7087-5p, mmu-miR-7088-3p, mmu-miR-7088-5p, mmu-miR-7089-3p, mmu-miR-7089-5p, mmu-miR-709, mmu-miR-7090-3p, mmu-miR-7090-5p, mmu-miR-7091-3p, mmu-miR-7091-5p, mmu-miR-7092-3p, mmu-miR-7092-5p, mmu-miR-7093-3p, mmu-miR-7093-5p, mmu-miR-7094-1-5p, mmu-miR-7094-3p, mmu-miR-7094b-2-5p, mmu-miR-710, mmu-miR-711, mmu-miR-7115-3p, mmu-miR-7115-5p, mmu-miR-7116-3p, mmu-miR-7116-5p, mmu-miR-7117-3p, mmu-miR-7117-5p, mmu-miR-7118-3p, mmu-miR-7118-5p, mmu-miR-7119-3p, mmu-miR-7119-5p, mmu-miR-712-3p, mmu-miR-712-5p, mmu-miR-713, mmu-miR-714, mmu-miR-717, mmu-miR-718, mmu-miR-719, mmu-miR-721, mmu-miR-7210-3p, mmu-miR-7210-5p, mmu-miR-7211-3p, mmu-miR-7211-5p, mmu-miR-7212-3p, mmu-miR-7212-5p, mmu-miR-7213-3p, mmu-miR-7213-5p, mmu-miR-7214-3p, mmu-miR-7214-5p, mmu-miR-7215-3p, mmu-miR-7215-5p, mmu-miR-7216-3p, mmu-miR-7216-5p, mmu-miR-7217-3p, mmu-miR-7217-5p, mmu-miR-7218-3p, mmu-miR-7218-5p, mmu-miR-7219-3p, mmu-miR-7219-5p, mmu-miR-7220-3p, mmu-miR-7220-5p, mmu-miR-7221-3p, mmu-miR-7221-5p, mmu-miR-7222-3p, mmu-miR-7222-5p, mmu-miR-7223-3p, mmu-miR-7223-5p, mmu-miR-7224-3p, mmu-miR-7224-5p, mmu-miR-7225-3p, mmu-miR-7225-5p, mmu-miR-7226-3p, mmu-miR-7226-5p, mmu-miR-7227-3p, mmu-miR-7227-5p, mmu-miR-7228-3p, mmu-miR-7228-5p, mmu-miR-7229-3p, mmu-miR-7229-5p, mmu-miR-7230-3p, mmu-miR-7230-5p, mmu-miR-7231-3p, mmu-miR-7231-5p, mmu-miR-7232-3p, mmu-miR-7232-5p, mmu-miR-7233-3p, mmu-miR-7233-5p, mmu-miR-7234-3p, mmu-miR-7234-5p, mmu-miR-7235-3p, mmu-miR-7235-5p, mmu-miR-7236-3p, mmu-miR-7236-5p, mmu-miR-7237-3p, mmu-miR-7237-5p, mmu-miR-7238-3p, mmu-miR-7238-5p, mmu-miR-7239-3p, mmu-miR-7239-5p, mmu-miR-7240-3p, mmu-miR-7240-5p, mmu-miR-7241-3p, mmu-miR-7241-5p, mmu-miR-7242-3p, mmu-miR-7242-5p, mmu-miR-7243-3p, mmu-miR-7243-5p, mmu-miR-741-3p, mmu-miR-741-5p, mmu-miR-742-3p, mmu-miR-742-5p, mmu-miR-743a-3p, mmu-miR-743a-5p, mmu-miR-743b-3p, mmu-miR-743b-5p, mmu-miR-744-3p, mmu-miR-744-5p, mmu-miR-7578, mmu-miR-758-3p, mmu-miR-758-5p, mmu-miR-759, mmu-miR-760-3p, mmu-miR-760-5p, mmu-miR-761, mmu-miR-762, mmu-miR-763, mmu-miR-764-3p, mmu-miR-764-5p, mmu-miR-7646-3p, mmu-miR-7646-5p, mmu-miR-7647-3p, mmu-miR-7647-5p, mmu-miR-7648-3p, mmu-miR-7648-5p, mmu-miR-7649-3p, mmu-miR-7649-5p, mmu-miR-7650-3p, mmu-miR-7650-5p, mmu-miR-7651-3p, mmu-miR-7651-5p, mmu-miR-7652-3p, mmu-miR-7652-5p, mmu-miR-7653-3p, mmu-miR-7653-5p, mmu-miR-7654-3p, mmu-miR-7654-5p, mmu-miR-7655-3p, mmu-miR-7655-5p, mmu-miR-7656-3p, mmu-miR-7656-5p, mmu-miR-7657-3p, mmu-miR-7657-5p, mmu-miR-7658-3p, mmu-miR-7658-5p, mmu-miR-7659-3p, mmu-miR-7659-5p, mmu-miR-7660-3p, mmu-miR-7660-5p, mmu-miR-7661-3p, mmu-miR-7661-5p, mmu-miR-7662-3p, mmu-miR-7662-5p, mmu-miR-7663-3p, mmu-miR-7663-5p, mmu-miR-7664-3p, mmu-miR-7664-5p, mmu-miR-7665-3p, mmu-miR-7665-5p, mmu-miR-7666-3p, mmu-miR-7666-5p, mmu-miR-7667-3p, mmu-miR-7667-5p, mmu-miR-7668-3p, mmu-miR-7668-5p, mmu-miR-7669-3p, mmu-miR-7669-5p, mmu-miR-767, mmu-miR-7670-3p, mmu-miR-7670-5p, mmu-miR-7671-3p, mmu-miR-7671-5p, mmu-miR-7672-3p, mmu-miR-7672-5p, mmu-miR-7673-3p, mmu-miR-7673-5p, mmu-miR-7674-3p, mmu-miR-7674-5p, mmu-miR-7675-3p, mmu-miR-7675-5p, mmu-miR-7676-3p, mmu-miR-7676-5p, mmu-miR-7677-3p, mmu-miR-7677-5p, mmu-miR-7678-3p, mmu-miR-7678-5p, mmu-miR-7679-3p, mmu-miR-7679-5p, mmu-miR-7680-3p, mmu-miR-7680-5p, mmu-miR-7681-3p, mmu-miR-7681-5p, mmu-miR-7682-3p, mmu-miR-7682-5p, mmu-miR-7683-3p, mmu-miR-7683-5p, mmu-miR-7684-3p, mmu-miR-7684-5p, mmu-miR-7685-3p, mmu-miR-7685-5p, mmu-miR-7686-3p, mmu-miR-7686-5p, mmu-miR-7687-3p, mmu-miR-7687-5p, mmu-miR-7688-3p, mmu-miR-7688-5p, mmu-miR-7689-3p, mmu-miR-7689-5p, mmu-miR-770-3p, mmu-miR-770-5p, mmu-miR-7a-1-3p, mmu-miR-7a-2-3p, mmu-miR-7a-5p, mmu-miR-7b-3p, mmu-miR-7b-5p, mmu-miR-802-3p, mmu-miR-802-5p, mmu-miR-804, mmu-miR-8090, mmu-miR-8091, mmu-miR-8092, mmu-miR-8093, mmu-miR-8094, mmu-miR-8095, mmu-miR-8096, mmu-miR-8097, mmu-miR-8098, mmu-miR-8099, mmu-miR-8100, mmu-miR-8101, mmu-miR-8102, mmu-miR-8103, mmu-miR-8104, mmu-miR-8105, mmu-miR-8106, mmu-miR-8107, mmu-miR-8108, mmu-miR-8109, mmu-miR-8110, mmu-miR-8111, mmu-miR-8112, mmu-miR-8113, mmu-miR-8114, mmu-miR-8115, mmu-miR-8116, mmu-miR-8117, mmu-miR-8118, mmu-miR-8119, mmu-miR-8120, mmu-miR-871-3p, mmu-miR-871-5p, mmu-miR-872-3p, mmu-miR-872-5p, mmu-miR-873a-3p, mmu-miR-873a-5p, mmu-miR-873b, mmu-miR-874-3p, mmu-miR-874-5p, mmu-miR-875-3p, mmu-miR-875-5p, mmu-miR-876-3p, mmu-miR-876-5p, mmu-miR-877-3p, mmu-miR-877-5p, mmu-miR-878-3p, mmu-miR-878-5p, mmu-miR-879-3p, mmu-miR-879-5p, mmu-miR-880-3p, mmu-miR-880-5p, mmu-miR-881-3p, mmu-miR-881-5p, mmu-miR-882, mmu-miR-883a-3p, mmu-miR-883a-5p, mmu-miR-883b-3p, mmu-miR-883b-5p, mmu-miR-9-3p, mmu-miR-9-5p, mmu-miR-92a-1-5p, mmu-miR-92a-2-5p, mmu-miR-92a-3p, mmu-miR-92b-3p, mmu-miR-92b-5p, mmu-miR-93-3p, mmu-miR-93-5p, mmu-miR-935, mmu-miR-96-3p, mmu-miR-96-5p, mmu-miR-9718, mmu-miR-9768-3p, mmu-miR-9768-5p, mmu-miR-9769-3p, mmu-miR-9769-5p, mmu-miR-98-3p, mmu-miR-98-5p, mmu-miR-99a-3p, mmu-miR-99a-5p, mmu-miR-99b-3p, mmu-miR-99b-5p, mmu-miR-9b-3p, mmu-miR-9b-5p, mne-miR-101, mne-miR-103, mne-miR-105, mne-miR-106a, mne-miR-106b, mne-miR-107, mne-miR-10b, mne-miR-125b, mne-miR-127, mne-miR-130a, mne-miR-133a, mne-miR-134, mne-miR-140, mne-miR-144, mne-miR-145, mne-miR-147, mne-miR-153, mne-miR-154, mne-miR-15a, mne-miR-15b, mne-miR-16, mne-miR-17-3p, mne-miR-17-5p, mne-miR-18, mne-miR-181a-3p, mne-miR-181a-5p, mne-miR-181b, mne-miR-183, mne-miR-184, mne-miR-187, mne-miR-188, mne-miR-194, mne-miR-197, mne-miR-198, mne-miR-199a, mne-miR-19a, mne-miR-19b, mne-miR-20, mne-miR-204, mne-miR-205, mne-miR-206, mne-miR-21, mne-miR-211, mne-miR-214, mne-miR-215, mne-miR-218, mne-miR-22, mne-miR-224, mne-miR-23a, mne-miR-24-3p, mne-miR-24-5p, mne-miR-25, mne-miR-26a, mne-miR-27a, mne-miR-28, mne-miR-29a, mne-miR-29b, mne-miR-30b, mne-miR-30c, mne-miR-30d, mne-miR-31, mne-miR-32, mne-miR-33, mne-miR-34a, mne-miR-7, mne-miR-9, mne-miR-92, mne-miR-93, mne-miR-96, mne-miR-99a, mse-bantam, mse-let-7a, mse-miR-100, mse-miR-1000, mse-miR-10a, mse-miR-10b, mse-miR-11, mse-miR-12, mse-miR-124, mse-miR-133, mse-miR-137, mse-miR-14, mse-miR-184, mse-miR-190, mse-miR-1926, mse-miR-193, mse-miR-1a, mse-miR-1b, mse-miR-210, mse-miR-252, mse-miR-2565, mse-miR-263a, mse-miR-263b, mse-miR-275, mse-miR-2755, mse-miR-276, mse-miR-2763, mse-miR-2765, mse-miR-2766, mse-miR-2767, mse-miR-2768, mse-miR-277, mse-miR-2779, mse-miR-278, mse-miR-2788, mse-miR-2796, mse-miR-2797a, mse-miR-2797b, mse-miR-279a, mse-miR-279b, mse-miR-279c, mse-miR-279d, mse-miR-281, mse-miR-282, mse-miR-283, mse-miR-285, mse-miR-2a, mse-miR-2b, mse-miR-306, mse-miR-307, mse-miR-308, mse-miR-31, mse-miR-316, mse-miR-317, mse-miR-3286, mse-miR-33, mse-miR-3338, mse-miR-3389, mse-miR-34, mse-miR-450, mse-miR-6093, mse-miR-6094, mse-miR-6095, mse-miR-6096, mse-miR-6097, mse-miR-6098, mse-miR-6099, mse-miR-6100, mse-miR-6101, mse-miR-7, mse-miR-71, mse-miR-745, mse-miR-750, mse-miR-79, mse-miR-8, mse-miR-87, mse-miR-929a, mse-miR-929b, mse-miR-92a, mse-miR-92b, mse-miR-932, mse-miR-9570, mse-miR-9571a, mse-miR-9571b, mse-miR-965, mse-miR-970, mse-miR-981, mse-miR-989, mse-miR-993, mse-miR-998, mse-miR-9a, mse-miR-9b, mse-miR-iab-4-5p, mtr-miR1507-3p, mtr-miR1507-5p, mtr-miR1509a-3p, mtr-miR1509a-5p, mtr-miR1509b, mtr-miR1510a-3p, mtr-miR1510a-5p, mtr-miR1510b-3p, mtr-miR1510b-5p, mtr-miR156a, mtr-miR156b-3p, mtr-miR156b-5p, mtr-miR156c-3p, mtr-miR156c-5p, mtr-miR156d-3p, mtr-miR156d-5p, mtr-miR156e, mtr-miR156f, mtr-miR156g-3p, mtr-miR156g-5p, mtr-miR156 h-3p, mtr-miR156 h-5p, mtr-miR156i-3p, mtr-miR156i-5p, mtr-miR156j, mtr-miR159a, mtr-miR159b, mtr-miR160a, mtr-miR160b, mtr-miR160c, mtr-miR160d, mtr-miR160e, mtr-miR160f, mtr-miR162, mtr-miR164a, mtr-miR164b, mtr-miR164c, mtr-miR164d, mtr-miR166a, mtr-miR166b, mtr-miR166c, mtr-miR166d, mtr-miR166e-3p, mtr-miR166e-5p, mtr-miR166f, mtr-miR166g-3p, mtr-miR166g-5p, mtr-miR167a, mtr-miR167b-3p, mtr-miR167b-5p, mtr-miR168a, mtr-miR168b, mtr-miR168c-3p, mtr-miR168c-5p, mtr-miR169a, mtr-miR169b, mtr-miR169c, mtr-miR169d-3p, mtr-miR169d-5p, mtr-miR169e-3p, mtr-miR169e-5p, mtr-miR169f, mtr-miR169g, mtr-miR169h, mtr-miR169i, mtr-miR169j, mtr-miR169k, mtr-miR1691-3p, mtr-miR1691-5p, mtr-miR171a, mtr-miR171b, mtr-miR171c, mtr-miR171d, mtr-miR171e-3p, mtr-miR171e-5p, mtr-miR171f, mtr-miR171g, mtr-miR171h, mtr-miR172a, mtr-miR172b, mtr-miR172c-3p, mtr-miR172c-5p, mtr-miR172d-3p, mtr-miR172d-5p, mtr-miR2086-3p, mtr-miR2086-5p, mtr-miR2087-3p, mtr-miR2087-5p, mtr-miR2088-3p, mtr-miR2088-5p, mtr-miR2089-3p, mtr-miR2089-5p, mtr-miR2111a-3p, mtr-miR2111a-5p, mtr-miR2111b, mtr-miR2111c, mtr-miR2111d-3p, mtr-miR2111d-5p, mtr-miR21Ile-3p, mtr-miR21Ile-5p, mtr-miR2111f, mtr-miR2111g-3p, mtr-miR2111g-5p, mtr-miR2111h, mtr-miR2111i, mtr-miR2111j, mtr-miR2111k, mtr-miR21111, mtr-miR2111m-3p, mtr-miR2111m-5p, mtr-miR2111n, mtr-miR21110, mtr-miR2118, mtr-miR2119, mtr-miR2199, mtr-miR2585a, mtr-miR2585b, mtr-miR2585c, mtr-miR2585d, mtr-miR2586a, mtr-miR2586b, mtr-miR2587a, mtr-miR2587b, mtr-miR2587c, mtr-miR2587d, mtr-miR2587e, mtr-miR2587f, mtr-miR2587g, mtr-miR2588a, mtr-miR2588b, mtr-miR2589, mtr-miR2590a, mtr-miR2590b, mtr-miR2590c, mtr-miR2590d, mtr-miR2590e, mtr-miR2590f, mtr-miR2590g, mtr-miR2590h, mtr-miR2590i, mtr-miR2590j, mtr-miR2591, mtr-miR2592a-3p, mtr-miR2592a-5p, mtr-miR2592a.2-3p, mtr-miR2592ab, mtr-miR2592ac, mtr-miR2592ad, mtr-miR2592ae, mtr-miR2592af, mtr-miR2592ah, mtr-miR2592ai, mtr-miR2592aj, mtr-miR2592al, mtr-miR2592 am, mtr-miR2592an, mtr-miR2592ao, mtr-miR2592ap, mtr-miR2592aq, mtr-miR2592ar, mtr-miR2592as, mtr-miR2592at, mtr-miR2592au, mtr-miR2592av, mtr-miR2592aw, mtr-miR2592ax, mtr-miR2592ay, mtr-miR2592az, mtr-miR2592b-3p, mtr-miR2592b-5p, mtr-miR2592ba, mtr-miR2592bb, mtr-miR2592bc, mtr-miR2592bd, mtr-miR2592be, mtr-miR2592bf, mtr-miR2592bg, mtr-miR2592bi, mtr-miR2592bj, mtr-miR2592bk, mtr-miR2592bl-3p, mtr-miR2592bl-5p, mtr-miR2592bm-3p, mtr-miR2592bm-5p, mtr-miR2592bn-3p, mtr-miR2592bn-5p, mtr-miR2592bo-3p, mtr-miR2592bo-5p, mtr-miR2592 bp-3p, mtr-miR2592 bp-5p, mtr-miR2592bq-3p, mtr-miR2592bq-5p, mtr-miR2592br-3p, mtr-miR2592br-5p, mtr-miR2592c, mtr-miR2592d-3p, mtr-miR2592d-5p, mtr-miR2592e-3p, mtr-miR2592e-5p, mtr-miR2592f, mtr-miR2592g, mtr-miR2592h, mtr-miR2592i, mtr-miR2592j, mtr-miR2592k, mtr-miR25921, mtr-miR2592m, mtr-miR2592n, mtr-miR25920-3p, mtr-miR25920-5p, mtr-miR2592p, mtr-miR2592q-3p, mtr-miR2592q-5p, mtr-miR2592r, mtr-miR2592s-3p, mtr-miR2592s-5p, mtr-miR2592t, mtr-miR2592u, mtr-miR2592v, mtr-miR2592w, mtr-miR2592x, mtr-miR2592y, mtr-miR2592z, mtr-miR2593a, mtr-miR2593b, mtr-miR2593c, mtr-miR2593d, mtr-miR2593e, mtr-miR2594, mtr-miR2595, mtr-miR2596, mtr-miR2597, mtr-miR2598, mtr-miR2599, mtr-miR2600a, mtr-miR2600b, mtr-miR2600c, mtr-miR2600d, mtr-miR2600e, mtr-miR2601, mtr-miR2602a, mtr-miR2602b, mtr-miR2603, mtr-miR2604, mtr-miR2605, mtr-miR2606a, mtr-miR2606b, mtr-miR2606c, mtr-miR2607, mtr-miR2608, mtr-miR2609a, mtr-miR2609b, mtr-miR2610a, mtr-miR2610b, mtr-miR2611, mtr-miR2612, mtr-miR2613, mtr-miR2614, mtr-miR2615a, mtr-miR2615b, mtr-miR2615c, mtr-miR2616, mtr-miR2617a, mtr-miR2617b, mtr-miR2617c, mtr-miR2618a, mtr-miR2618b, mtr-miR2619a, mtr-miR2619b-3p, mtr-miR2619b-5p, mtr-miR2620, mtr-miR2621, mtr-miR2622, mtr-miR2623, mtr-miR2624, mtr-miR2625, mtr-miR2626, mtr-miR2627, mtr-miR2628, mtr-miR2629a, mtr-miR2629b, mtr-miR2629c, mtr-miR2629d, mtr-miR2629e, mtr-miR2629f, mtr-miR2629g, mtr-miR2629h, mtr-miR2630a, mtr-miR2630b, mtr-miR2630c, mtr-miR2630d, mtr-miR2630e, mtr-miR2630f, mtr-miR2630g, mtr-miR2630h, mtr-miR2630i, mtr-miR2630j, mtr-miR2630k, mtr-miR26301, mtr-miR2630m, mtr-miR2630n, mtr-miR26300, mtr-miR2630p, mtr-miR2630q, mtr-miR2630r, mtr-miR2630s, mtr-miR2630t, mtr-miR2630u, mtr-miR2630v, mtr-miR2630w, mtr-miR2630x, mtr-miR2630y, mtr-miR2631, mtr-miR2632a, mtr-miR2632b, mtr-miR2632c, mtr-miR2633, mtr-miR2634, mtr-miR2635, mtr-miR2636, mtr-miR2637, mtr-miR2638a, mtr-miR2638b, mtr-miR2639, mtr-miR2640, mtr-miR2641, mtr-miR2642, mtr-miR2643a, mtr-miR2643b-3p, mtr-miR2643b-5p, mtr-miR2644, mtr-miR2645, mtr-miR2646a, mtr-miR2646b, mtr-miR2647a, mtr-miR2647b, mtr-miR2647c, mtr-miR2648, mtr-miR2649, mtr-miR2650, mtr-miR2651, mtr-miR2652a, mtr-miR2652b, mtr-miR2652c, mtr-miR2652d, mtr-miR2652e, mtr-miR2652f, mtr-miR2652g, mtr-miR2652h, mtr-miR2652i, mtr-miR2652j, mtr-miR2652k, mtr-miR26521, mtr-miR2652m, mtr-miR2653a, mtr-miR2653b, mtr-miR2653c, mtr-miR2653d, mtr-miR2654, mtr-miR2655a, mtr-miR2655b, mtr-miR2655c, mtr-miR2655d, mtr-miR2655e, mtr-miR2655f, mtr-miR2655g, mtr-miR2655h, mtr-miR2655i, mtr-miR2655j, mtr-miR2655k, mtr-miR26551, mtr-miR2655m, mtr-miR2655n, mtr-miR26550, mtr-miR2656a, mtr-miR2656b, mtr-miR2656c, mtr-miR2656d, mtr-miR2656e, mtr-miR2657, mtr-miR2658, mtr-miR2659a, mtr-miR2659b, mtr-miR2659c, mtr-miR2659d, mtr-miR2659e, mtr-miR2659f, mtr-miR2659g, mtr-miR2659h, mtr-miR2659i, mtr-miR2659j, mtr-miR2659k, mtr-miR2660, mtr-miR2661, mtr-miR2662, mtr-miR2663, mtr-miR2664a, mtr-miR2664b, mtr-miR2665, mtr-miR2666, mtr-miR2667a, mtr-miR2667b, mtr-miR2668, mtr-miR2669a, mtr-miR2669b, mtr-miR2670a, mtr-miR2670b, mtr-miR2670c, mtr-miR2670d, mtr-miR2670e, mtr-miR2670f, mtr-miR2670g, mtr-miR2671a, mtr-miR2671b, mtr-miR2671c, mtr-miR2671d, mtr-miR2671e, mtr-miR2671f, mtr-miR2671g, mtr-miR2671h, mtr-miR2671i, mtr-miR2671j, mtr-miR2672, mtr-miR2673a, mtr-miR2673b, mtr-miR2674, mtr-miR2675, mtr-miR2676a, mtr-miR2676b, mtr-miR2676c, mtr-miR2676d, mtr-miR2676e, mtr-miR2676f, mtr-miR2677, mtr-miR2678, mtr-miR2679a, mtr-miR2679b, mtr-miR2679c, mtr-miR2680a, mtr-miR2680b, mtr-miR2680c, mtr-miR2680d, mtr-miR2680e, mtr-miR319a-3p, mtr-miR319a-5p, mtr-miR319b-3p, mtr-miR319b-5p, mtr-miR319c-3p, mtr-miR319c-5p, mtr-miR319d-3p, mtr-miR319d-5p, mtr-miR390, mtr-miR393a, mtr-miR393b-3p, mtr-miR393b-5p, mtr-miR395a, mtr-miR395b, mtr-miR395c, mtr-miR395d, mtr-miR395e, mtr-miR395f, mtr-miR395g, mtr-miR395h, mtr-miR395i, mtr-miR395j, mtr-miR395k, mtr-miR3951, mtr-miR395m, mtr-miR395n, mtr-miR3950, mtr-miR396a-3p, mtr-miR396a-5p, mtr-miR396b-3p, mtr-miR396b-5p, mtr-miR396c, mtr-miR397-3p, mtr-miR397-5p, mtr-miR398a-3p, mtr-miR398a-5p, mtr-miR398b, mtr-miR398c, mtr-miR399a, mtr-miR399b, mtr-miR399c, mtr-miR399d, mtr-miR399e, mtr-miR399f, mtr-miR399g, mtr-miR399h, mtr-miR399i, mtr-miR399j, mtr-miR399k, mtr-miR3991, mtr-miR399m, mtr-miR399n, mtr-miR3990, mtr-miR399p, mtr-miR399q, mtr-miR399r, mtr-miR399s-3p, mtr-miR399s-5p, mtr-miR399t-3p, mtr-miR399t-5p, mtr-miR408-3p, mtr-miR408-5p, mtr-miR4414a-3p, mtr-miR4414a-5p, mtr-miR4414b, mtr-miR482-3p, mtr-miR482-5p, mtr-miR5037a, mtr-miR5037b, mtr-miR5037c, mtr-miR5204, mtr-miR5205a, mtr-miR5205b, mtr-miR5205c, mtr-miR5205d, mtr-miR5206a, mtr-miR5206b, mtr-miR5207, mtr-miR5208a, mtr-miR5208b, mtr-miR5208c, mtr-miR5208d, mtr-miR5209, mtr-miR5210, mtr-miR5211, mtr-miR5212-3p, mtr-miR5212-5p, mtr-miR5213-3p, mtr-miR5213-5p, mtr-miR5214-3p, mtr-miR5214-5p, mtr-miR5215, mtr-miR5216a, mtr-miR5216b, mtr-miR5217, mtr-miR5218, mtr-miR5219, mtr-miR5221, mtr-miR5222, mtr-miR5223, mtr-miR5224a, mtr-miR5224b, mtr-miR5225a, mtr-miR5225b, mtr-miR5225c, mtr-miR5226, mtr-miR5227, mtr-miR5228, mtr-miR5229a, mtr-miR5229b, mtr-miR5230, mtr-miR5231, mtr-miR5232, mtr-miR5233, mtr-miR5234, mtr-miR5235a, mtr-miR5235b, mtr-miR5236a, mtr-miR5236b, mtr-miR5236c, mtr-miR5236d, mtr-miR5236e, mtr-miR5237, mtr-miR5238, mtr-miR5239, mtr-miR5240, mtr-miR5241a, mtr-miR5241b, mtr-miR5241c, mtr-miR5242, mtr-miR5243, mtr-miR5244, mtr-miR5245, mtr-miR5246, mtr-miR5247, mtr-miR5248, mtr-miR5249, mtr-miR5250, mtr-miR5251, mtr-miR5252, mtr-miR5253, mtr-miR5254, mtr-miR5255, mtr-miR5256, mtr-miR5257, mtr-miR5258, mtr-miR5259, mtr-miR5260, mtr-miR5261, mtr-miR5262, mtr-miR5263, mtr-miR5264, mtr-miR5265, mtr-miR5266, mtr-miR5267a, mtr-miR5267b, mtr-miR5267c, mtr-miR5267d, mtr-miR5267e, mtr-miR5267f, mtr-miR5267g, mtr-miR5267h, mtr-miR5267i, mtr-miR5267j, mtr-miR5267k, mtr-miR52671, mtr-miR5267m, mtr-miR5267n, mtr-miR52670, mtr-miR5268a, mtr-miR5268b, mtr-miR5269a, mtr-miR5269b, mtr-miR5270a, mtr-miR5270b, mtr-miR5271a, mtr-miR5271b, mtr-miR5271c, mtr-miR5271d, mtr-miR5271e, mtr-miR5272a, mtr-miR5272b, mtr-miR5272c, mtr-miR5272d, mtr-miR5272e, mtr-miR5272f, mtr-miR5273, mtr-miR5274a, mtr-miR5274b-3p, mtr-miR5274b-5p, mtr-miR5275, mtr-miR5276, mtr-miR5277, mtr-miR5278, mtr-miR5279, mtr-miR5280, mtr-miR5281a, mtr-miR5281b, mtr-miR5281c, mtr-miR5281d, mtr-miR5281e, mtr-miR5281f, mtr-miR5282, mtr-miR5283, mtr-miR5284a, mtr-miR5284b, mtr-miR5284c, mtr-miR5284d, mtr-miR5284e, mtr-miR5284f, mtr-miR5284g, mtr-miR5284h, mtr-miR5285a, mtr-miR5285b, mtr-miR5285c, mtr-miR5286a, mtr-miR5286b, mtr-miR5287a, mtr-miR5287b, mtr-miR5288, mtr-miR5289a, mtr-miR5289b, mtr-miR5290, mtr-miR5291a, mtr-miR5291b, mtr-miR5291c, mtr-miR5292a, mtr-miR5292b, mtr-miR5293, mtr-miR5294a, mtr-miR5294b, mtr-miR5294c, mtr-miR5295a, mtr-miR5295b, mtr-miR5295c, mtr-miR5295d, mtr-miR5296, mtr-miR5297, mtr-miR5298a, mtr-miR5298b, mtr-miR5298c, mtr-miR5298d, mtr-miR5299, mtr-miR530, mtr-miR5554a-3p, mtr-miR5554a-5p, mtr-miR5554b-3p, mtr-miR5554b-5p, mtr-miR5554c-3p, mtr-miR5554c-5p, mtr-miR5555-3p, mtr-miR5555-5p, mtr-miR5556-3p, mtr-miR5556-5p, mtr-miR5557-3p, mtr-miR5557-5p, mtr-miR5558-3p, mtr-miR5558-5p, mtr-miR5559-3p, mtr-miR5559-5p, mtr-miR5560-3p, mtr-miR5560-5p, mtr-miR5561-3p, mtr-miR5561-5p, mtr-miR5562-3p, mtr-miR5562-5p, mtr-miR5563-3p, mtr-miR5563-5p, mtr-miR5740, mtr-miR5741a, mtr-miR5741b, mtr-miR5741c, mtr-miR5741d, mtr-miR5741e, mtr-miR5742, mtr-miR5743a, mtr-miR5743b, mtr-miR5744, mtr-miR5745a, mtr-miR5745b, mtr-miR5746, mtr-miR5747, mtr-miR5748, mtr-miR5749, mtr-miR5750, mtr-miR5751, mtr-miR5752a, mtr-miR5752b, mtr-miR5753, mtr-miR5754, mtr-miR5755, mtr-miR5756, mtr-miR5757, mtr-miR5758, mtr-miR5759, mtr-miR5760, mtr-miR7696a-3p, mtr-miR7696a-5p, mtr-miR7696b-3p, mtr-miR7696b-5p, mtr-miR7696c-3p, mtr-miR7696c-5p, mtr-miR7696d-3p, mtr-miR7696d-5p, mtr-miR7697-3p, mtr-miR7697-5p, mtr-miR7698-3p, mtr-miR7698-5p, mtr-miR7699-3p, mtr-miR7699-5p, mtr-miR7700-5p, mtr-miR7701-3p, mtr-miR7701-5p, mze-let-7a, mze-let-7b, mze-let-7c, mze-let-7d, mze-let-7e, mze-let-7g, mze-let-7h, mze-let-7i, mze-miR-1, mze-miR-100, mze-miR-101a, mze-miR-101b, mze-miR-103, mze-miR-10544, mze-miR-10545, mze-miR-10552, mze-miR-10553, mze-miR-10554, mze-miR-10566, mze-miR-10567, mze-miR-106, mze-miR-107, mze-miR-10a, mze-miR-10b, mze-miR-10c, mze-miR-10d, mze-miR-122, mze-miR-124, mze-miR-125a, mze-miR-125b, mze-miR-125c, mze-miR-126, mze-miR-128, mze-miR-129, mze-miR-130, mze-miR-132a, mze-miR-132b, mze-miR-133a, mze-miR-133b, mze-miR-135a, mze-miR-135b, mze-miR-135c, mze-miR-135c-3p, mze-miR-135d, mze-miR-137, mze-miR-138, mze-miR-1388, mze-miR-139, mze-miR-140, mze-miR-142, mze-miR-144, mze-miR-146, mze-miR-147, mze-miR-148-5p, mze-miR-150, mze-miR-153a, mze-miR-153b, mze-miR-153c, mze-miR-155, mze-miR-15a, mze-miR-15b, mze-miR-15c-3p, mze-miR-15c-5p, mze-miR-16a, mze-miR-16b, mze-miR-17, mze-miR-181a, mze-miR-181b, mze-miR-182, mze-miR-183, mze-miR-184a, mze-miR-184b, mze-miR-187, mze-miR-18a, mze-miR-18b, mze-miR-190a, mze-miR-190b, mze-miR-192, mze-miR-193, mze-miR-194a, mze-miR-194b, mze-miR-196a, mze-miR-196b, mze-miR-199, mze-miR-19a, mze-miR-19b, mze-miR-19c, mze-miR-19d, mze-miR-200a, mze-miR-200b, mze-miR-203, mze-miR-204, mze-miR-205, mze-miR-206, mze-miR-20a, mze-miR-20b, mze-miR-21, mze-miR-212a, mze-miR-212b, mze-miR-214, mze-miR-216a, mze-miR-216b, mze-miR-2187a, mze-miR-2187b, mze-miR-2188, mze-miR-218a, mze-miR-218b, mze-miR-219a, mze-miR-219b, mze-miR-219c, mze-miR-221, mze-miR-222, mze-miR-22a, mze-miR-22b, mze-miR-22c, mze-miR-23a, mze-miR-23b, mze-miR-23c, mze-miR-24a, mze-miR-24b, mze-miR-25, mze-miR-26a, mze-miR-26b, mze-miR-27a, mze-miR-27b, mze-miR-27d-3p, mze-miR-27d-5p, mze-miR-27e, mze-miR-29a, mze-miR-29b, mze-miR-301a, mze-miR-301b, mze-miR-30a, mze-miR-30b, mze-miR-30c, mze-miR-30d, mze-miR-31, mze-miR-3120, mze-miR-338, mze-miR-33a, mze-miR-33b, mze-miR-34, mze-miR-365, mze-miR-375, mze-miR-429b, mze-miR-449a, mze-miR-449b, mze-miR-455, mze-miR-456, mze-miR-457, mze-miR-458, mze-miR-460, mze-miR-462, mze-miR-489, mze-miR-499, mze-miR-551, mze-miR-7132a-3p, mze-miR-7132a-5p, mze-miR-7132b-3p, mze-miR-7132b-5p, mze-miR-7133, mze-miR-7147, mze-miR-722, mze-miR-723a, mze-miR-723b, mze-miR-724, mze-miR-725, mze-miR-726, mze-miR-727, mze-miR-728, mze-miR-730, mze-miR-731, mze-miR-734, mze-miR-7a, mze-miR-7b, mze-miR-8159, mze-miR-8160b, mze-miR-9, mze-miR-92a, mze-miR-92b, mze-miR-93, mze-miR-96, mze-miR-99a, mze-miR-99b, nbr-let-7a, nbr-let-7b, nbr-let-7c, nbr-let-7d, nbr-let-7e, nbr-let-7g, nbr-let-7h, nbr-let-7i, nbr-miR-1, nbr-miR-100, nbr-miR-101a, nbr-miR-101b, nbr-miR-103, nbr-miR-10544, nbr-miR-10553, nbr-miR-10568, nbr-miR-10569, nbr-miR-10570, nbr-miR-10571, nbr-miR-10572, nbr-miR-107, nbr-miR-10a, nbr-miR-10b, nbr-miR-10c, nbr-miR-10d, nbr-miR-122, nbr-miR-124, nbr-miR-125a, nbr-miR-125b, nbr-miR-126, nbr-miR-129, nbr-miR-130, nbr-miR-132a, nbr-miR-132b, nbr-miR-133a, nbr-miR-133b, nbr-miR-135a, nbr-miR-135b, nbr-miR-135c-3p, nbr-miR-135c-5p, nbr-miR-135d, nbr-miR-137, nbr-miR-138, nbr-miR-1388, nbr-miR-139, nbr-miR-140, nbr-miR-142, nbr-miR-144, nbr-miR-147, nbr-miR-148-3p, nbr-miR-148-5p, nbr-miR-150, nbr-miR-153a, nbr-miR-153b, nbr-miR-153c, nbr-miR-155, nbr-miR-15a, nbr-miR-15b, nbr-miR-15c-3p, nbr-miR-15c-5p, nbr-miR-16a, nbr-miR-16b, nbr-miR-17, nbr-miR-181a, nbr-miR-181b, nbr-miR-182, nbr-miR-183, nbr-miR-184a, nbr-miR-184b, nbr-miR-187, nbr-miR-18a, nbr-miR-18b, nbr-miR-190a, nbr-miR-190b, nbr-miR-192, nbr-miR-193, nbr-miR-194a, nbr-miR-194b, nbr-miR-196a, nbr-miR-196b, nbr-miR-199, nbr-miR-19a, nbr-miR-19b, nbr-miR-19c, nbr-miR-19d, nbr-miR-200a, nbr-miR-200b, nbr-miR-203, nbr-miR-204, nbr-miR-205, nbr-miR-206, nbr-miR-20a, nbr-miR-20b, nbr-miR-21, nbr-miR-212a, nbr-miR-212b, nbr-miR-214, nbr-miR-216a, nbr-miR-216b, nbr-miR-2187a, nbr-miR-2187b, nbr-miR-2188, nbr-miR-218a, nbr-miR-218b, nbr-miR-219a, nbr-miR-219b, nbr-miR-219c, nbr-miR-221, nbr-miR-222, nbr-miR-22a, nbr-miR-22b, nbr-miR-22c, nbr-miR-23a, nbr-miR-23b, nbr-miR-23c, nbr-miR-24a, nbr-miR-24b, nbr-miR-24c, nbr-miR-25, nbr-miR-26a, nbr-miR-26b, nbr-miR-27a, nbr-miR-27b, nbr-miR-27d-3p, nbr-miR-27d-5p, nbr-miR-27e, nbr-miR-29a, nbr-miR-29b, nbr-miR-29c, nbr-miR-301a, nbr-miR-301b, nbr-miR-30a, nbr-miR-30b, nbr-miR-30c, nbr-miR-30d, nbr-miR-31, nbr-miR-3120, nbr-miR-338, nbr-miR-33a, nbr-miR-33b, nbr-miR-34, nbr-miR-365, nbr-miR-375, nbr-miR-429b, nbr-miR-449a, nbr-miR-449b, nbr-miR-455, nbr-miR-456, nbr-miR-457, nbr-miR-458, nbr-miR-460, nbr-miR-462, nbr-miR-489, nbr-miR-499, nbr-miR-551, nbr-miR-7, nbr-miR-7132a, nbr-miR-7132a-5p, nbr-miR-7132b, nbr-miR-7133-3p, nbr-miR-7133-5p, nbr-miR-7147, nbr-miR-722, nbr-miR-723a, nbr-miR-723b, nbr-miR-724, nbr-miR-725, nbr-miR-726, nbr-miR-727, nbr-miR-728, nbr-miR-730, nbr-miR-731, nbr-miR-734, nbr-miR-8159, nbr-miR-8160, nbr-miR-9, nbr-miR-92a, nbr-miR-92b, nbr-miR-93, nbr-miR-96, nbr-miR-99a, nbr-miR-99b, ngi-let-7, ngi-miR-1, ngi-miR-100, ngi-miR-133, ngi-miR-137, ngi-miR-13a, ngi-miR-14, ngi-miR-184, ngi-miR-2, ngi-miR-210, ngi-miR-219, ngi-miR-252, ngi-miR-263b, ngi-miR-275, ngi-miR-276, ngi-miR-277, ngi-miR-281, ngi-miR-283, ngi-miR-29b, ngi-miR-305, ngi-miR-317, ngi-miR-31a, ngi-miR-34, ngi-miR-375, ngi-miR-927, ngi-miR-929, ngi-miR-92a, ngi-miR-932, ngi-miR-993, ngi-miR-9a, ngi-miR-iab-4, ngi-miR-iab-8, nle-let-7a, nle-let-7c, nle-let-7e, nle-let-7f, nle-let-7g, nle-let-7i, nle-miR-100, nle-miR-101, nle-miR-103a, nle-miR-106b, nle-miR-107, nle-miR-10a, nle-miR-10b, nle-miR-125a, nle-miR-125b, nle-miR-126, nle-miR-1260b, nle-miR-127, nle-miR-128, nle-miR-1296, nle-miR-1307, nle-miR-130a, nle-miR-132, nle-miR-136, nle-miR-138, nle-miR-140, nle-miR-143, nle-miR-145, nle-miR-146a, nle-miR-146b, nle-miR-148a, nle-miR-148b, nle-miR-149, nle-miR-151, nle-miR-152, nle-miR-153, nle-miR-155, nle-miR-15a, nle-miR-15b, nle-miR-16, nle-miR-17, nle-miR-181a, nle-miR-181b, nle-miR-181c, nle-miR-182, nle-miR-186, nle-miR-190, nle-miR-191, nle-miR-192, nle-miR-193b, nle-miR-195, nle-miR-196b, nle-miR-199a, nle-miR-199b, nle-miR-19a, nle-miR-19b, nle-miR-20, nle-miR-21, nle-miR-210, nle-miR-212, nle-miR-214, nle-miR-218, nle-miR-22, nle-miR-221, nle-miR-222, nle-miR-224, nle-miR-23b, nle-miR-24, nle-miR-2467, nle-miR-25, nle-miR-26a, nle-miR-27b, nle-miR-28, nle-miR-29a, nle-miR-29b, nle-miR-29c, nle-miR-301a, nle-miR-30a, nle-miR-30b, nle-miR-30c, nle-miR-30d, nle-miR-30e, nle-miR-31, nle-miR-320a, nle-miR-320b, nle-miR-320c, nle-miR-324, nle-miR-331, nle-miR-340, nle-miR-345, nle-miR-34a, nle-miR-34c, nle-miR-361, nle-miR-365, nle-miR-374a, nle-miR-378a, nle-miR-378d, nle-miR-381, nle-miR-3925, nle-miR-409, nle-miR-410, nle-miR-421, nle-miR-423, nle-miR-424, nle-miR-450a, nle-miR-450b, nle-miR-454, nle-miR-455, nle-miR-4670, nle-miR-4714, nle-miR-484, nle-miR-497, nle-miR-501, nle-miR-502a, nle-miR-502b, nle-miR-505, nle-miR-532, nle-miR-542, nle-miR-549, nle-miR-561, nle-miR-5699, nle-miR-582, nle-miR-628, nle-miR-660, nle-miR-671, nle-miR-7180, nle-miR-769, nle-miR-877, nle-miR-887, nle-miR-92a, nle-miR-92b, nle-miR-93, nle-miR-942, nle-miR-98, nle-miR-99a, nlo-miR-10, nlo-miR-12, nlo-miR-125, nlo-miR-133, nlo-miR-137, nlo-miR-13a, nlo-miR-184, nlo-miR-2, nlo-miR-219, nlo-miR-252, nlo-miR-263b, nlo-miR-275, nlo-miR-276, nlo-miR-277, nlo-miR-281, nlo-miR-282, nlo-miR-305, nlo-miR-315, nlo-miR-31a, nlo-miR-34, nlo-miR-7, nlo-miR-8, nlo-miR-927, nlo-miR-929, nlo-miR-92a, nlo-miR-932, nlo-miR-993, nlo-miR-9a, nta-miR1446, nta-miR156a, nta-miR156b, nta-miR156c, nta-miR156d, nta-miR156e, nta-miR156f, nta-miR156g, nta-miR156h, nta-miR156i, nta-miR156j, nta-miR159, nta-miR160a, nta-miR160b, nta-miR160c, nta-miR160d, nta-miR162a, nta-miR162b, nta-miR164a, nta-miR164b, nta-miR164c, nta-miR166a, nta-miR166b, nta-miR166c, nta-miR166d, nta-miR166e, nta-miR166f, nta-miR166g, nta-miR166h, nta-miR167a, nta-miR167b, nta-miR167c, nta-miR167d, nta-miR167e, nta-miR168a, nta-miR168b, nta-miR168c, nta-miR168d, nta-miR168e, nta-miR169a, nta-miR169b, nta-miR169c, nta-miR169d, nta-miR169e, nta-miR169f, nta-miR169g, nta-miR169h, nta-miR169i, nta-miR169j, nta-miR169k, nta-miR1691, nta-miR169m, nta-miR1690, nta-miR169p, nta-miR169q, nta-miR169r, nta-miR169s, nta-miR169t, nta-miR171a, nta-miR171b, nta-miR171c, nta-miR172a, nta-miR172b, nta-miR172c, nta-miR172d, nta-miR172e, nta-miR172f, nta-miR172g, nta-miR172h, nta-miR172i, nta-miR172j, nta-miR1919, nta-miR319a, nta-miR319b, nta-miR390a, nta-miR390b, nta-miR390c, nta-miR394, nta-miR395a, nta-miR395b, nta-miR395c, nta-miR396a, nta-miR396b, nta-miR396c, nta-miR397, nta-miR398, nta-miR399a, nta-miR399b, nta-miR399c, nta-miR399d, nta-miR399e, nta-miR399f, nta-miR399g, nta-miR408, nta-miR477a, nta-miR477b, nta-miR479a, nta-miR479b, nta-miR482a, nta-miR482b-3p, nta-miR482b-5p, nta-miR482c, nta-miR482d, nta-miR5303a, nta-miR5303b, nta-miR5303c, nta-miR6019a, nta-miR6019b, nta-miR6020a-3p, nta-miR6020a-5p, nta-miR6020b, nta-miR6021, nta-miR6024, nta-miR6025a, nta-miR6025b, nta-miR6025c, nta-miR6025d, nta-miR6025e, nta-miR6144, nta-miR6145a, nta-miR6145b, nta-miR6145c, nta-miR6145d, nta-miR6145e, nta-miR6145f, nta-miR6146a, nta-miR6146b, nta-miR6147, nta-miR6148a, nta-miR6148b, nta-miR6149a, nta-miR6149b, nta-miR6150, nta-miR6151a, nta-miR6151b, nta-miR6151c, nta-miR6151d, nta-miR6151e, nta-miR6151f, nta-miR6151g, nta-miR6151h, nta-miR6151i, nta-miR6152a, nta-miR6152b, nta-miR6153, nta-miR6154a, nta-miR6154b, nta-miR6155, nta-miR6156, nta-miR6157, nta-miR6158a, nta-miR6158b, nta-miR6158c, nta-miR6159, nta-miR6160, nta-miR6161a, nta-miR6161b, nta-miR6161c, nta-miR6161d, nta-miR6162, nta-miR6163, nta-miR6164a, nta-miR6164b, nta-miR827, nve-miR-100-3p, nve-miR-100-5p, nve-miR-2022-3p, nve-miR-2022-5p, nve-miR-2023-3p, nve-miR-2023-5p, nve-miR-2024a-3p, nve-miR-2024a-5p, nve-miR-2024b-3p, nve-miR-2024b-5p, nve-miR-2024c-3p, nve-miR-2024c-5p, nve-miR-2024d, nve-miR-2024e-3p, nve-miR-2024e-5p, nve-miR-2024f-3p, nve-miR-2024f-5p, nve-miR-2024g-3p, nve-miR-2024g-5p, nve-miR-2025-3p, nve-miR-2025-5p, nve-miR-2026-3p, nve-miR-2026-5p, nve-miR-2027-3p, nve-miR-2027-5p, nve-miR-2028-3p, nve-miR-2028-5p, nve-miR-2029-3p, nve-miR-2029-5p, nve-miR-2030-3p, nve-miR-2030-5p, nve-miR-2031-3p, nve-miR-2031-5p, nve-miR-2032a-3p, nve-miR-2032a-5p, nve-miR-2032b-3p, nve-miR-2032b-5p, nve-miR-2033-3p, nve-miR-2033-5p, nve-miR-2034-3p, nve-miR-2034-5p, nve-miR-2035-3p, nve-miR-2035-5p, nve-miR-2036-3p, nve-miR-2036-5p, nve-miR-2037-3p, nve-miR-2037-5p, nve-miR-2038-3p, nve-miR-2038-5p, nve-miR-2039-3p, nve-miR-2039-5p, nve-miR-2040a-3p, nve-miR-2040a-5p, nve-miR-2040b-3p, nve-miR-2040b-5p, nve-miR-2041a-3p, nve-miR-2041a-5p, nve-miR-2041b-3p, nve-miR-2041b-5p, nve-miR-2042-3p, nve-miR-2042-5p, nve-miR-2043-2-3p, nve-miR-2043-3p, nve-miR-2043-5p, nve-miR-2044-3p, nve-miR-2044-5p, nve-miR-2045-3p, nve-miR-2045-5p, nve-miR-2046-3p, nve-miR-2046-5p, nve-miR-2047-3p, nve-miR-2047-5p, nve-miR-2048-3p, nve-miR-2048-5p, nve-miR-2049-3p, nve-miR-2049-5p, nve-miR-2050-3p, nve-miR-2050-5p, nve-miR-2051-3p, nve-miR-2051-5p, nve-miR-9412, nve-miR-9413, nve-miR-9414-3p, nve-miR-9414-5p, nve-miR-9415, nve-miR-9416, nve-miR-9417, nve-miR-9418, nve-miR-9419, nve-miR-9420, nve-miR-9421, nve-miR-9422, nve-miR-9423, nve-miR-9424a, nve-miR-9424b, nve-miR-9425, nve-miR-9426, nve-miR-9427, nve-miR-9428, nve-miR-9429-3p, nve-miR-9429-5p, nve-miR-9430, nve-miR-9431, nve-miR-9432, nve-miR-9433, nve-miR-9434, nve-miR-9435, nve-miR-9436, nve-miR-9437, nve-miR-9438, nve-miR-9439, nve-miR-9440, nve-miR-9441, nve-miR-9442, nve-miR-9443, nve-miR-9444, nve-miR-9445, nve-miR-9446, nve-miR-9447, nve-miR-9448, nve-miR-9449, nve-miR-9450, nve-miR-9451, nve-miR-9452, nve-miR-9453, nve-miR-9454, nve-miR-9455, nve-miR-9456, nve-miR-9457, nve-miR-9458, nve-miR-9459, nve-miR-9460, nve-miR-9461, nve-miR-9462, nve-miR-9463, nve-miR-9464, nve-miR-9465, nve-miR-9466, nve-miR-9467, nve-miR-9468, nvi-bantam, nvi-let-7, nvi-miR-1, nvi-miR-10, nvi-miR-100, nvi-miR-12, nvi-miR-124, nvi-miR-125, nvi-miR-133, nvi-miR-137, nvi-miR-13a, nvi-miR-13b, nvi-miR-14, nvi-miR-184, nvi-miR-190, nvi-miR-210, nvi-miR-219, nvi-miR-252, nvi-miR-263b, nvi-miR-275, nvi-miR-276, nvi-miR-277, nvi-miR-279, nvi-miR-2796, nvi-miR-281, nvi-miR-282, nvi-miR-283, nvi-miR-29b, nvi-miR-2a, nvi-miR-2b, nvi-miR-2c, nvi-miR-305, nvi-miR-307, nvi-miR-315, nvi-miR-317, nvi-miR-31a, nvi-miR-33, nvi-miR-34, nvi-miR-3477, nvi-miR-3478, nvi-miR-375, nvi-miR-7, nvi-miR-71, nvi-miR-8, nvi-miR-927, nvi-miR-928, nvi-miR-929, nvi-miR-92a, nvi-miR-932, nvi-miR-993, nvi-miR-9a, nvi-miR-iab-4, nvi-miR-iab-8, oan-let-7b-3p, oan-let-7b-5p, oan-let-7c-3p, oan-let-7c-5p, oan-let-7d-3p, oan-let-7d-5p, oan-let-7e-3p, oan-let-7e-5p, oan-let-7f-1-3p, oan-let-7f-2-3p, oan-let-7f-5p, oan-let-7g-3p, oan-let-7g-5p, oan-miR-100-3p, oan-miR-100-5p, oan-miR-101, oan-miR-103-1-5p, oan-miR-103-2-5p, oan-miR-103-3p, oan-miR-106-3p, oan-miR-106-5p, oan-miR-107-3p, oan-miR-107-5p, oan-miR-10a-3p, oan-miR-10a-5p, oan-miR-10b-3p, oan-miR-10b-5p, oan-miR-122-3p, oan-miR-122-5p, oan-miR-124a-1-3p, oan-miR-124a-2-3p, oan-miR-124a-5p, oan-miR-125-3p, oan-miR-125-5p, oan-miR-126-3p, oan-miR-126-5p, oan-miR-128-3p, oan-miR-128-5p, oan-miR-129-3p, oan-miR-129-5p, oan-miR-130a, oan-miR-130b-3p, oan-miR-130b-5p, oan-miR-130c-3p, oan-miR-130c-5p, oan-miR-1325, oan-miR-1326, oan-miR-1327-3p, oan-miR-1327-5p, oan-miR-1328, oan-miR-1329-3p, oan-miR-1329-5p, oan-miR-1330, oan-miR-1331, oan-miR-1332-3p, oan-miR-1332-5p, oan-miR-1333, oan-miR-1334, oan-miR-1335, oan-miR-1336, oan-miR-1337, oan-miR-1338-3p, oan-miR-1338-5p, oan-miR-1339-3p, oan-miR-1339-5p, oan-miR-133a-3p, oan-miR-133a-5p, oan-miR-133b-3p, oan-miR-133b-5p, oan-miR-133c, oan-miR-1340-3p, oan-miR-1340-5p, oan-miR-1341, oan-miR-1342, oan-miR-1343-3p, oan-miR-1343-5p, oan-miR-1344, oan-miR-1345, oan-miR-1346, oan-miR-1347, oan-miR-1348, oan-miR-1349, oan-miR-1350, oan-miR-1351-3p, oan-miR-1351-5p, oan-miR-1352, oan-miR-1353, oan-miR-1354-3p, oan-miR-1354-5p, oan-miR-1355-3p, oan-miR-1355-5p, oan-miR-1356, oan-miR-1357, oan-miR-1358, oan-miR-1359, oan-miR-135a-3p, oan-miR-135b-1-3p, oan-miR-135b-2-3p, oan-miR-135b-5p, oan-miR-1360, oan-miR-1361-3p, oan-miR-1361-5p, oan-miR-1362-3p, oan-miR-1362-5p, oan-miR-1363-3p, oan-miR-1363-5p, oan-miR-1364, oan-miR-1365-3p, oan-miR-1365-5p, oan-miR-1366, oan-miR-1367-3p, oan-miR-1367-5p, oan-miR-1368, oan-miR-1369, oan-miR-1370, oan-miR-1371, oan-miR-1372, oan-miR-1373, oan-miR-1374, oan-miR-1375-3p, oan-miR-1375-5p, oan-miR-1376-3p, oan-miR-1376-5p, oan-miR-1377, oan-miR-1378, oan-miR-1379, oan-miR-137a-3p, oan-miR-137a-5p, oan-miR-137b-3p, oan-miR-137b-5p, oan-miR-138-3p, oan-miR-138-5p, oan-miR-1380-3p, oan-miR-1380-5p, oan-miR-1381-3p, oan-miR-1381-5p, oan-miR-1382, oan-miR-1383, oan-miR-1384, oan-miR-1385, oan-miR-1386, oan-miR-1387, oan-miR-1388-3p, oan-miR-1388-5p, oan-miR-1389-3p, oan-miR-1389-5p, oan-miR-139-3p, oan-miR-139-5p, oan-miR-1390-3p, oan-miR-1390-5p, oan-miR-1391-3p, oan-miR-1391-5p, oan-miR-1392-3p, oan-miR-1392-5p, oan-miR-1393, oan-miR-1394, oan-miR-1395-3p, oan-miR-1395-5p, oan-miR-1396, oan-miR-1397-3p, oan-miR-1397-5p, oan-miR-1398-3p, oan-miR-1398-5p, oan-miR-1399, oan-miR-140-3p, oan-miR-140-5p, oan-miR-1400-3p, oan-miR-1400-5p, oan-miR-1401-3p, oan-miR-1401-5p, oan-miR-1402, oan-miR-1403, oan-miR-1404, oan-miR-1405-3p, oan-miR-1405-5p, oan-miR-1406-3p, oan-miR-1406-5p, oan-miR-1407, oan-miR-1408, oan-miR-1409-3p, oan-miR-1409-5p, oan-miR-1410, oan-miR-1411, oan-miR-1412, oan-miR-1413-3p, oan-miR-1413-5p, oan-miR-1414-3p, oan-miR-1414-5p, oan-miR-1415, oan-miR-1416, oan-miR-1417-3p, oan-miR-1417-5p, oan-miR-1418-3p, oan-miR-1418-5p, oan-miR-1419a-3p, oan-miR-1419a-5p, oan-miR-1419b-1-3p, oan-miR-1419b-2-3p, oan-miR-1419b-5p, oan-miR-1419c-3p, oan-miR-1419c-5p, oan-miR-1419d-3p, oan-miR-1419d-5p, oan-miR-1419e-3p, oan-miR-1419e-5p, oan-miR-1419f-3p, oan-miR-1419f-5p, oan-miR-1419g-3p, oan-miR-1419g-5p, oan-miR-142-3p, oan-miR-142-5p, oan-miR-1420a-3p, oan-miR-1420a-5p, oan-miR-1420b-3p, oan-miR-1420b-5p, oan-miR-1420c-3p, oan-miR-1420c-5p, oan-miR-1420d-3p, oan-miR-1420d-5p, oan-miR-1420e-3p, oan-miR-1420e-5p, oan-miR-1420f, oan-miR-1420g-3p, oan-miR-1420g-5p, oan-miR-1421a-3p, oan-miR-1421a-5p, oan-miR-1421aa-3p, oan-miR-1421aa-5p, oan-miR-1421ab-2-3p, oan-miR-1421ab-3p, oan-miR-1421ab-5p, oan-miR-1421ac-2-3p, oan-miR-1421ac-3p, oan-miR-1421ac-5p, oan-miR-1421ad-3p, oan-miR-1421ad-5p, oan-miR-1421ae, oan-miR-1421af-3p, oan-miR-1421af-5p, oan-miR-1421ag-3p, oan-miR-1421ag-5p, oan-miR-1421ah-3p, oan-miR-1421ah-5p, oan-miR-1421ai-3p, oan-miR-1421ai-5p, oan-miR-1421aj-3p, oan-miR-1421aj-5p, oan-miR-1421ak-3p, oan-miR-1421ak-5p, oan-miR-1421al-3p, oan-miR-1421al-5p, oan-miR-1421 am, oan-miR-1421an-3p, oan-miR-1421an-5p, oan-miR-1421b-3p, oan-miR-1421b-5p, oan-miR-1421c-3p, oan-miR-1421c-5p, oan-miR-1421d-3p, oan-miR-1421d-5p, oan-miR-1421e-3p, oan-miR-1421e-5p, oan-miR-1421f-3p, oan-miR-1421f-5p, oan-miR-1421g-1-3p, oan-miR-1421g-2-3p, oan-miR-1421g-5p, oan-miR-1421 h-3p, oan-miR-1421 h-5p, oan-miR-1421i-1-3p, oan-miR-1421i-2-3p, oan-miR-1421i-3-3p, oan-miR-1421i-5p, oan-miR-1421j-3p, oan-miR-1421j-5p, oan-miR-1421k-1-3p, oan-miR-1421k-3p, oan-miR-1421k-5p, oan-miR-14211-1-3p, oan-miR-14211-2-3p, oan-miR-14211-5p, oan-miR-1421m-3p, oan-miR-1421m-5p, oan-miR-1421n-1-3p, oan-miR-1421n-2-3p, oan-miR-1421n-5p, oan-miR-14210-3p, oan-miR-14210-5p, oan-miR-1421p-3p, oan-miR-1421p-5p, oan-miR-1421q-3p, oan-miR-1421q-5p, oan-miR-1421r-3p, oan-miR-1421r-5p, oan-miR-1421s-3p, oan-miR-1421s-5p, oan-miR-1421t-3p, oan-miR-1421t-5p, oan-miR-1421u-5p, oan-miR-1421v-3p, oan-miR-1421v-5p, oan-miR-1421w-3p, oan-miR-1421w-5p, oan-miR-1421x-3p, oan-miR-1421x-5p, oan-miR-1421y-2-3p, oan-miR-1421y-3-3p, oan-miR-1421y-5p, oan-miR-1421z-3p, oan-miR-1421z-5p, oan-miR-1422a-3p, oan-miR-1422a-5p, oan-miR-1422b-1-5p, oan-miR-1422b-2-5p, oan-miR-1422b-3p, oan-miR-1422c-3p, oan-miR-1422c-5p, oan-miR-1422d-3p, oan-miR-1422e-3p, oan-miR-1422e-5p, oan-miR-1422f-3p, oan-miR-1422f-5p, oan-miR-1422g-3p, oan-miR-1422g-5p, oan-miR-1422 h-3p, oan-miR-1422 h-5p, oan-miR-1422i-3p, oan-miR-1422i-5p, oan-miR-1422j-3p, oan-miR-1422j-5p, oan-miR-1422k-3p, oan-miR-1422k-5p, oan-miR-14221, oan-miR-1422m-3p, oan-miR-1422m-5p, oan-miR-1422n-3p, oan-miR-1422n-5p, oan-miR-14220-3p, oan-miR-14220-5p, oan-miR-1422p-3p, oan-miR-1422p-5p, oan-miR-1422q-3p, oan-miR-1422q-5p, oan-miR-143-3p, oan-miR-143-5p, oan-miR-144-3p, oan-miR-144-5p, oan-miR-145-3p, oan-miR-145-5p, oan-miR-146a-3p, oan-miR-146a-5p, oan-miR-146b-3p, oan-miR-146b-5p, oan-miR-147, oan-miR-148-3p, oan-miR-148-5p, oan-miR-150-3p, oan-miR-150-5p, oan-miR-153-1-5p, oan-miR-153-2-5p, oan-miR-153-3p, oan-miR-155-3p, oan-miR-155-5p, oan-miR-15a-3p, oan-miR-15a-5p, oan-miR-15b-3p, oan-miR-15b-5p, oan-miR-15c-3p, oan-miR-15c-5p, oan-miR-16a-3p, oan-miR-16a-5p, oan-miR-16b-3p, oan-miR-16b-5p, oan-miR-16c-3p, oan-miR-16c-5p, oan-miR-17-3p, oan-miR-17-5p, oan-miR-18-3p, oan-miR-18-5p, oan-miR-181a-1-3p, oan-miR-181a-2-3p, oan-miR-181a-3-3p, oan-miR-181a-5p, oan-miR-181b-1-3p, oan-miR-181b-2-3p, oan-miR-181b-5p, oan-miR-181c-3p, oan-miR-181c-5p, oan-miR-182-3p, oan-miR-182-5p, oan-miR-183, oan-miR-184, oan-miR-186-3p, oan-miR-186-5p, oan-miR-187-3p, oan-miR-187-5p, oan-miR-190a-3p, oan-miR-190a-5p, oan-miR-190b, oan-miR-191-3p, oan-miR-191-5p, oan-miR-192-3p, oan-miR-192-5p, oan-miR-193-3p, oan-miR-193-5p, oan-miR-194-3p, oan-miR-194-5p, oan-miR-196a-3p, oan-miR-196a-5p, oan-miR-196b-3p, oan-miR-196b-5p, oan-miR-199-3p, oan-miR-199-5p, oan-miR-19a-3p, oan-miR-19a-5p, oan-miR-19b-1-5p, oan-miR-19b-2-5p, oan-miR-19b-3p, oan-miR-1a-3p, oan-miR-1a-5p, oan-miR-1b, oan-miR-200a-3p, oan-miR-200a-5p, oan-miR-200b-3p, oan-miR-200b-5p, oan-miR-204-3p, oan-miR-204-5p, oan-miR-205-3p, oan-miR-205-5p, oan-miR-206-3p, oan-miR-206-5p, oan-miR-208-3p, oan-miR-208-5p, oan-miR-20a-1-3p, oan-miR-20a-2-3p, oan-miR-20a-5p, oan-miR-20b-3p, oan-miR-20b-5p, oan-miR-21-3p, oan-miR-21-5p, oan-miR-214-3p, oan-miR-214-5p, oan-miR-215-3p, oan-miR-215-5p, oan-miR-216a-3p, oan-miR-216a-5p, oan-miR-216b-3p, oan-miR-216b-5p, oan-miR-217-3p, oan-miR-217-5p, oan-miR-218-3p, oan-miR-218-5p, oan-miR-219-3p, oan-miR-219-5p, oan-miR-22-3p, oan-miR-22-5p, oan-miR-221-3p, oan-miR-221-5p, oan-miR-222a-3p, oan-miR-222a-5p, oan-miR-222b-3p, oan-miR-222b-5p, oan-miR-223-3p, oan-miR-223-5p, oan-miR-23a-3p, oan-miR-23a-5p, oan-miR-23b-3p, oan-miR-23b-5p, oan-miR-24-1-5p, oan-miR-24-2-5p, oan-miR-24-3p, oan-miR-26-1-3p, oan-miR-26-2-3p, oan-miR-26-5p, oan-miR-27a-3p, oan-miR-27a-5p, oan-miR-27b-3p, oan-miR-27b-5p, oan-miR-2985-3p, oan-miR-2985-5p, oan-miR-29a-1-5p, oan-miR-29a-2-5p, oan-miR-29a-3p, oan-miR-29b-3p, oan-miR-29b-5p, oan-miR-301-3p, oan-miR-301-5p, oan-miR-302-3p, oan-miR-302-5p, oan-miR-30a-3p, oan-miR-30a-5p, oan-miR-30b-3p, oan-miR-30b-5p, oan-miR-30c-1-3p, oan-miR-30c-2-3p, oan-miR-30c-5p, oan-miR-30d-3p, oan-miR-30d-5p, oan-miR-30e-3p, oan-miR-30e-5p, oan-miR-30f, oan-miR-31-3p, oan-miR-31-5p, oan-miR-32-3p, oan-miR-32-5p, oan-miR-33-5p, oan-miR-33a-3p, oan-miR-33b-3p, oan-miR-34a-3p, oan-miR-34a-5p, oan-miR-34b-3p, oan-miR-34b-5p, oan-miR-363-3p, oan-miR-363-5p, oan-miR-365-1-5p, oan-miR-365-2-5p, oan-miR-365-3p, oan-miR-383-3p, oan-miR-383-5p, oan-miR-425-3p, oan-miR-425-5p, oan-miR-429-3p, oan-miR-429-5p, oan-miR-449a, oan-miR-449b-3p, oan-miR-449b-5p, oan-miR-449c, oan-miR-451, oan-miR-454-3p, oan-miR-454-5p, oan-miR-458-3p, oan-miR-458-5p, oan-miR-460-3p, oan-miR-460-5p, oan-miR-490-3p, oan-miR-490-5p, oan-miR-499-3p, oan-miR-499-5p, oan-miR-551-3p, oan-miR-551-5p, oan-miR-590-3p, oan-miR-590-5p, oan-miR-7-1-3p, oan-miR-7-3p, oan-miR-7-5p, oan-miR-7400-3p, oan-miR-7400-5p, oan-miR-7401-3p, oan-miR-7401-5p, oan-miR-7402-3p, oan-miR-7402-5p, oan-miR-7403-3p, oan-miR-7403-5p, oan-miR-7404-3p, oan-miR-7404-5p, oan-miR-7405-3p, oan-miR-7405-5p, oan-miR-7406-3p, oan-miR-7406-5p, oan-miR-7407-3p, oan-miR-7407-5p, oan-miR-7408-3p, oan-miR-7408-5p, oan-miR-7409-3p, oan-miR-7409-5p, oan-miR-7410-3p, oan-miR-7410-5p, oan-miR-7411-3p, oan-miR-7411-5p, oan-miR-7412-3p, oan-miR-7412-5p, oan-miR-7413-3p, oan-miR-7413-5p, oan-miR-7414-3p, oan-miR-7414-5p, oan-miR-7415-3p, oan-miR-7415-5p, oan-miR-7416-3p, oan-miR-7416-5p, oan-miR-7417-3p, oan-miR-7417-5p, oan-miR-7418-3p, oan-miR-7418-5p, oan-miR-7419-3p, oan-miR-7419-5p, oan-miR-7420-3p, oan-miR-7420-5p, oan-miR-7421-3p, oan-miR-7421-5p, oan-miR-7422a-3p, oan-miR-7422a-5p, oan-miR-7422b-3p, oan-miR-7422b-5p, oan-miR-7423-3p, oan-miR-7423-5p, oan-miR-7424-3p, oan-miR-7424-5p, oan-miR-7425-3p, oan-miR-7425-5p, oan-miR-7426-3p, oan-miR-7426-5p, oan-miR-7427-3p, oan-miR-7427-5p, oan-miR-7428-3p, oan-miR-7428-5p, oan-miR-7429-3p, oan-miR-7429-5p, oan-miR-7430-3p, oan-miR-7430-5p, oan-miR-7431-3p, oan-miR-7431-5p, oan-miR-7432-3p, oan-miR-7432-5p, oan-miR-7433-3p, oan-miR-7433-5p, oan-miR-7434-3p, oan-miR-7434-5p, oan-miR-7435-3p, oan-miR-7435-5p, oan-miR-7436-3p, oan-miR-7436-5p, oan-miR-802-3p, oan-miR-802-5p, oan-miR-873-3p, oan-miR-873-5p, oan-miR-875-3p, oan-miR-875-5p, oan-miR-9-3p, oan-miR-9-5p, oan-miR-92a-1-5p, oan-miR-92a-2-5p, oan-miR-92a-3p, oan-miR-92b, oan-miR-92c, oan-miR-92d, oan-miR-96-3p, oan-miR-96-5p, oan-miR-98, oan-miR-99-3p, oan-miR-99-5p, oar-let-7a, oar-let-7b, oar-let-7c, oar-let-7d, oar-let-7f, oar-let-7g, oar-let-7i, oar-miR-103, oar-miR-106a, oar-miR-106b, oar-miR-107, oar-miR-10a, oar-miR-10b, oar-miR-1185-3p, oar-miR-1185-5p, oar-miR-1193-3p, oar-miR-1193-5p, oar-miR-1197-3p, oar-miR-1197-5p, oar-miR-125b, oar-miR-127, oar-miR-133, oar-miR-134-3p, oar-miR-134-5p, oar-miR-136, oar-miR-143, oar-miR-148a, oar-miR-150, oar-miR-152, oar-miR-154a-3p, oar-miR-154a-5p, oar-miR-154b-3p, oar-miR-154b-5p, oar-miR-16b, oar-miR-17-5p, oar-miR-181a, oar-miR-191, oar-miR-194, oar-miR-199a-3p, oar-miR-19b, oar-miR-200a, oar-miR-200b, oar-miR-200c, oar-miR-21, oar-miR-218a, oar-miR-22-3p, oar-miR-221, oar-miR-23a, oar-miR-23b, oar-miR-25, oar-miR-26a, oar-miR-26b, oar-miR-27a, oar-miR-299-3p, oar-miR-299-5p, oar-miR-29a, oar-miR-29b, oar-miR-30a-3p, oar-miR-30a-5p, oar-miR-30b, oar-miR-30c, oar-miR-30d, oar-miR-323a-3p, oar-miR-323a-5p, oar-miR-323b, oar-miR-323c, oar-miR-329a-3p, oar-miR-329a-5p, oar-miR-329b-3p, oar-miR-329b-5p, oar-miR-362, oar-miR-369-3p, oar-miR-369-5p, oar-miR-370-3p, oar-miR-370-5p, oar-miR-374a, oar-miR-374b, oar-miR-376a-3p, oar-miR-376a-5p, oar-miR-376b-3p, oar-miR-376b-5p, oar-miR-376c-3p, oar-miR-376c-5p, oar-miR-376d, oar-miR-376e-3p, oar-miR-376e-5p, oar-miR-377-3p, oar-miR-377-5p, oar-miR-379-3p, oar-miR-379-5p, oar-miR-380-3p, oar-miR-380-5p, oar-miR-381-3p, oar-miR-381-5p, oar-miR-382-3p, oar-miR-382-5p, oar-miR-3955-3p, oar-miR-3955-5p, oar-miR-3956-3p, oar-miR-3956-5p, oar-miR-3957-3p, oar-miR-3957-5p, oar-miR-3958-3p, oar-miR-3958-5p, oar-miR-3959-3p, oar-miR-3959-5p, oar-miR-409-3p, oar-miR-409-5p, oar-miR-410-3p, oar-miR-410-5p, oar-miR-411a-3p, oar-miR-411a-5p, oar-miR-411b-3p, oar-miR-411b-5p, oar-miR-412-3p, oar-miR-412-5p, oar-miR-431, oar-miR-432, oar-miR-433-3p, oar-miR-433-5p, oar-miR-485-3p, oar-miR-485-5p, oar-miR-487a-3p, oar-miR-487a-5p, oar-miR-487b-3p, oar-miR-487b-5p, oar-miR-493-3p, oar-miR-493-5p, oar-miR-494-3p, oar-miR-494-5p, oar-miR-495-3p, oar-miR-495-5p, oar-miR-496-3p, oar-miR-496-5p, oar-miR-539-3p, oar-miR-539-5p, oar-miR-541-3p, oar-miR-541-5p, oar-miR-543-3p, oar-miR-543-5p, oar-miR-544-3p, oar-miR-544-5p, oar-miR-654-3p, oar-miR-654-5p, oar-miR-655-3p, oar-miR-655-5p, oar-miR-665-3p, oar-miR-665-5p, oar-miR-668-3p, oar-miR-668-5p, oar-miR-758-3p, oar-miR-758-5p, oar-miR-99a, ocu-let-7a-2-3p, ocu-let-7a-3p, ocu-let-7a-5p, ocu-let-7b-3p, ocu-let-7b-5p, ocu-let-7c-3p, ocu-let-7c-5p, ocu-let-7d-3p, ocu-let-7d-5p, ocu-let-7f-2-3p, ocu-let-7f-3p, ocu-let-7f-5p, ocu-let-7g-3p, ocu-let-7g-5p, ocu-let-7i-3p, ocu-let-7i-5p, ocu-miR-1-3p, ocu-miR-1-5p, ocu-miR-100-3p, ocu-miR-100-5p, ocu-miR-101-2-5p, ocu-miR-101-3p, ocu-miR-101-5p, ocu-miR-103a-2-5p, ocu-miR-103a-3p, ocu-miR-103a-5p, ocu-miR-105a-2-3p, ocu-miR-105a-3p, ocu-miR-105a-5p, ocu-miR-105b-3p, ocu-miR-105b-5p, ocu-miR-106a-3p, ocu-miR-106a-5p, ocu-miR-106b-3p, ocu-miR-106b-5p, ocu-miR-107-3p, ocu-miR-107-5p, ocu-miR-10a-3p, ocu-miR-10a-5p, ocu-miR-10b-3p, ocu-miR-10b-5p, ocu-miR-1197-3p, ocu-miR-1197-5p, ocu-miR-12090-3p, ocu-miR-12090-5p, ocu-miR-12091-3p, ocu-miR-12091-5p, ocu-miR-12092-3p, ocu-miR-12092-5p, ocu-miR-12093-3p, ocu-miR-12093-5p, ocu-miR-12094-3p, ocu-miR-12094-5p, ocu-miR-12095-3p, ocu-miR-12095-5p, ocu-miR-122-3p, ocu-miR-122-5p, ocu-miR-124-3p, ocu-miR-124-5p, ocu-miR-1249-3p, ocu-miR-1249-5p, ocu-miR-1251-3p, ocu-miR-1251-5p, ocu-miR-125b-2-3p, ocu-miR-125b-3p, ocu-miR-125b-5p, ocu-miR-1264-3p, ocu-miR-1264-5p, ocu-miR-127-3p, ocu-miR-127-5p, ocu-miR-1277-3p, ocu-miR-1277-5p, ocu-miR-128a-3p, ocu-miR-128a-5p, ocu-miR-128b-3p, ocu-miR-128b-5p, ocu-miR-129-2-3p, ocu-miR-129-3p, ocu-miR-129-5p, ocu-miR-1296-3p, ocu-miR-1296-5p, ocu-miR-1298-3p, ocu-miR-1298-5p, ocu-miR-1307-3p, ocu-miR-1307-5p, ocu-miR-130a-3p, ocu-miR-130a-5p, ocu-miR-130b-3p, ocu-miR-130b-5p, ocu-miR-133a-3p, ocu-miR-133a-5p, ocu-miR-133b-3p, ocu-miR-133b-5p, ocu-miR-134-3p, ocu-miR-134-5p, ocu-miR-1343-3p, ocu-miR-1343-5p, ocu-miR-135a-2-3p, ocu-miR-135a-3p, ocu-miR-135a-5p, ocu-miR-135b-3p, ocu-miR-135b-5p, ocu-miR-136-3p, ocu-miR-136-5p, ocu-miR-137-3p, ocu-miR-137-5p, ocu-miR-138-2-3p, ocu-miR-138-3p, ocu-miR-138-5p, ocu-miR-139-3p, ocu-miR-139-5p, ocu-miR-140-3p, ocu-miR-140-5p, ocu-miR-141-3p, ocu-miR-141-5p, ocu-miR-142-3p, ocu-miR-142-5p, ocu-miR-143-3p, ocu-miR-143-5p, ocu-miR-144-3p, ocu-miR-144-5p, ocu-miR-145-3p, ocu-miR-145-5p, ocu-miR-146a-3p, ocu-miR-146a-5p, ocu-miR-146b-3p, ocu-miR-146b-5p, ocu-miR-147-3p, ocu-miR-147-5p, ocu-miR-148a-3p, ocu-miR-148a-5p, ocu-miR-148b-3p, ocu-miR-148b-5p, ocu-miR-150-3p, ocu-miR-150-5p, ocu-miR-151-3p, ocu-miR-151-5p, ocu-miR-152-3p, ocu-miR-152-5p, ocu-miR-153-3p, ocu-miR-153-5p, ocu-miR-154-3p, ocu-miR-154-5p, ocu-miR-155-3p, ocu-miR-155-5p, ocu-miR-15a-3p, ocu-miR-15a-5p, ocu-miR-15b-3p, ocu-miR-15b-5p, ocu-miR-16a-3p, ocu-miR-16a-5p, ocu-miR-16b-3p, ocu-miR-16b-5p, ocu-miR-17-3p, ocu-miR-17-5p, ocu-miR-181a-2-3p, ocu-miR-181a-3p, ocu-miR-181a-5p, ocu-miR-181b-2-3p, ocu-miR-181b-3p, ocu-miR-181b-5p, ocu-miR-181c-3p, ocu-miR-181c-5p, ocu-miR-181d-3p, ocu-miR-181d-5p, ocu-miR-182-3p, ocu-miR-182-5p, ocu-miR-183-3p, ocu-miR-183-5p, ocu-miR-184-3p, ocu-miR-184-5p, ocu-miR-185-3p, ocu-miR-185-5p, ocu-miR-186-3p, ocu-miR-186-5p, ocu-miR-187-3p, ocu-miR-187-5p, ocu-miR-188-3p, ocu-miR-188-5p, ocu-miR-18a-3p, ocu-miR-18a-5p, ocu-miR-18b-3p, ocu-miR-18b-5p, ocu-miR-190a-3p, ocu-miR-190a-5p, ocu-miR-190b-3p, ocu-miR-190b-5p, ocu-miR-191-3p, ocu-miR-191-5p, ocu-miR-1911-3p, ocu-miR-1911-5p, ocu-miR-193a-3p, ocu-miR-193a-5p, ocu-miR-193b-3p, ocu-miR-193b-5p, ocu-miR-194-3p, ocu-miR-194-5p, ocu-miR-195-3p, ocu-miR-195-5p, ocu-miR-196a-2-3p, ocu-miR-196a-2-5p, ocu-miR-196a-3p, ocu-miR-196a-5p, ocu-miR-196b-3p, ocu-miR-196b-5p, ocu-miR-197-3p, ocu-miR-197-5p, ocu-miR-199a-3p, ocu-miR-199a-5p, ocu-miR-19a-3p, ocu-miR-19a-5p, ocu-miR-19b-2-5p, ocu-miR-19b-3p, ocu-miR-19b-5p, ocu-miR-200c-3p, ocu-miR-200c-5p, ocu-miR-204-3p, ocu-miR-204-5p, ocu-miR-205-3p, ocu-miR-205-5p, ocu-miR-206-3p, ocu-miR-206-5p, ocu-miR-208a-3p, ocu-miR-208a-5p, ocu-miR-208b-3p, ocu-miR-208b-5p, ocu-miR-20a-3p, ocu-miR-20a-5p, ocu-miR-20b-3p, ocu-miR-20b-5p, ocu-miR-21-3p, ocu-miR-21-5p, ocu-miR-211-3p, ocu-miR-211-5p, ocu-miR-212-3p, ocu-miR-212-5p, ocu-miR-214-3p, ocu-miR-214-5p, ocu-miR-215-3p, ocu-miR-215-5p, ocu-miR-216a-3p, ocu-miR-216a-5p, ocu-miR-216b-3p, ocu-miR-216b-5p, ocu-miR-217-3p, ocu-miR-217-5p, ocu-miR-218-2-3p, ocu-miR-218-3p, ocu-miR-218-5p, ocu-miR-219a-3p, ocu-miR-219a-5p, ocu-miR-219b-3p, ocu-miR-219b-5p, ocu-miR-22-3p, ocu-miR-22-5p, ocu-miR-221-3p, ocu-miR-221-5p, ocu-miR-222-3p, ocu-miR-222-5p, ocu-miR-223-3p, ocu-miR-223-5p, ocu-miR-224-3p, ocu-miR-224-5p, ocu-miR-2355-3p, ocu-miR-2355-5p, ocu-miR-2387-3p, ocu-miR-2387-5p, ocu-miR-23b-3p, ocu-miR-23b-5p, ocu-miR-24-2-5p, ocu-miR-24-3p, ocu-miR-24-5p, ocu-miR-25-3p, ocu-miR-25-5p, ocu-miR-26a-3p, ocu-miR-26a-5p, ocu-miR-26b-3p, ocu-miR-26b-5p, ocu-miR-27b-3p, ocu-miR-27b-5p, ocu-miR-28-3p, ocu-miR-28-5p, ocu-miR-299-3p, ocu-miR-299-5p, ocu-miR-29a-3p, ocu-miR-29a-5p, ocu-miR-29b-2-5p, ocu-miR-29b-3p, ocu-miR-29b-5p, ocu-miR-29c-3p, ocu-miR-29c-5p, ocu-miR-301a-3p, ocu-miR-301a-5p, ocu-miR-301b-3p, ocu-miR-301b-5p, ocu-miR-302a-3p, ocu-miR-302a-5p, ocu-miR-302b-3p, ocu-miR-302b-5p, ocu-miR-302c-3p, ocu-miR-302c-5p, ocu-miR-302d-3p, ocu-miR-302d-5p, ocu-miR-3059-3p, ocu-miR-3059-5p, ocu-miR-3085-3p, ocu-miR-3085-5p, ocu-miR-30a-3p, ocu-miR-30a-5p, ocu-miR-30b-3p, ocu-miR-30b-5p, ocu-miR-30c-2-3p, ocu-miR-30c-3p, ocu-miR-30c-5p, ocu-miR-30d-3p, ocu-miR-30d-5p, ocu-miR-30e-3p, ocu-miR-30e-5p, ocu-miR-31-3p, ocu-miR-31-5p, ocu-miR-32-3p, ocu-miR-32-5p, ocu-miR-323a-3p, ocu-miR-323a-5p, ocu-miR-324-3p, ocu-miR-324-5p, ocu-miR-325-3p, ocu-miR-325-5p, ocu-miR-326-3p, ocu-miR-326-5p, ocu-miR-328-3p, ocu-miR-328-5p, ocu-miR-329-3p, ocu-miR-329-5p, ocu-miR-331-3p, ocu-miR-331-5p, ocu-miR-335-3p, ocu-miR-335-5p, ocu-miR-337-3p, ocu-miR-337-5p, ocu-miR-33a-3p, ocu-miR-33a-5p, ocu-miR-340-3p, ocu-miR-340-5p, ocu-miR-342-3p, ocu-miR-342-5p, ocu-miR-346-5p, ocu-miR-34a-3p, ocu-miR-34a-5p, ocu-miR-34b-3p, ocu-miR-34b-5p, ocu-miR-34c-3p, ocu-miR-34c-5p, ocu-miR-350-3p, ocu-miR-350-5p, ocu-miR-361-3p, ocu-miR-361-5p, ocu-miR-3613-3p, ocu-miR-3613-5p, ocu-miR-362-3p, ocu-miR-362-5p, ocu-miR-363-3p, ocu-miR-363-5p, ocu-miR-365-2-5p, ocu-miR-365-3p, ocu-miR-365-5p, ocu-miR-367-3p, ocu-miR-367-5p, ocu-miR-369-3p, ocu-miR-369-5p, ocu-miR-370-3p, ocu-miR-370-5p, ocu-miR-371-3p, ocu-miR-371-5p, ocu-miR-373-3p, ocu-miR-374a-3p, ocu-miR-374a-5p, ocu-miR-374b-3p, ocu-miR-374b-5p, ocu-miR-375-3p, ocu-miR-375-5p, ocu-miR-376a-3p, ocu-miR-376a-5p, ocu-miR-376b-3p, ocu-miR-376b-5p, ocu-miR-376c-2-3p, ocu-miR-376c-3p, ocu-miR-376c-5p, ocu-miR-377-3p, ocu-miR-377-5p, ocu-miR-378-3p, ocu-miR-378-5p, ocu-miR-379-3p, ocu-miR-379-5p, ocu-miR-381-3p, ocu-miR-381-5p, ocu-miR-382-3p, ocu-miR-382-5p, ocu-miR-383-3p, ocu-miR-383-5p, ocu-miR-384-3p, ocu-miR-384-5p, ocu-miR-409-3p, ocu-miR-409-5p, ocu-miR-410-3p, ocu-miR-410-5p, ocu-miR-411-3p, ocu-miR-411-5p, ocu-miR-421-3p, ocu-miR-421-5p, ocu-miR-423-3p, ocu-miR-423-5p, ocu-miR-424-3p, ocu-miR-424-5p, ocu-miR-425-3p, ocu-miR-425-5p, ocu-miR-432-3p, ocu-miR-432-5p, ocu-miR-433-3p, ocu-miR-433-5p, ocu-miR-448-3p, ocu-miR-448-5p, ocu-miR-449a-3p, ocu-miR-449a-5p, ocu-miR-449b-3p, ocu-miR-449b-5p, ocu-miR-450a-2-3p, ocu-miR-450a-3p, ocu-miR-450a-5p, ocu-miR-450b-3p, ocu-miR-450b-5p, ocu-miR-451-5p, ocu-miR-452-3p, ocu-miR-452-5p, ocu-miR-455-3p, ocu-miR-455-5p, ocu-miR-485-3p, ocu-miR-485-5p, ocu-miR-486-3p, ocu-miR-486-5p, ocu-miR-487a-3p, ocu-miR-487a-5p, ocu-miR-487b-3p, ocu-miR-487b-5p, ocu-miR-488-3p, ocu-miR-488-5p, ocu-miR-489-3p, ocu-miR-489-5p, ocu-miR-490-3p, ocu-miR-490-5p, ocu-miR-491-3p, ocu-miR-491-5p, ocu-miR-495-3p, ocu-miR-495-5p, ocu-miR-496-3p, ocu-miR-496-5p, ocu-miR-497-3p, ocu-miR-497-5p, ocu-miR-498, ocu-miR-499-3p, ocu-miR-499-5p, ocu-miR-500-3p, ocu-miR-500-5p, ocu-miR-501-3p, ocu-miR-501-5p, ocu-miR-502a-3p, ocu-miR-502a-5p, ocu-miR-502b-3p, ocu-miR-502b-5p, ocu-miR-503-3p, ocu-miR-503-5p, ocu-miR-504-3p, ocu-miR-504-5p, ocu-miR-505-3p, ocu-miR-505-5p, ocu-miR-506-3p, ocu-miR-506-5p, ocu-miR-507-3p, ocu-miR-507-5p, ocu-miR-508-3p, ocu-miR-508-5p, ocu-miR-509a-3p, ocu-miR-509a-5p, ocu-miR-509b-3p, ocu-miR-509b-5p, ocu-miR-509c-3p, ocu-miR-509c-5p, ocu-miR-512a-3p, ocu-miR-512a-5p, ocu-miR-512b-3p, ocu-miR-512b-5p, ocu-miR-513-3p, ocu-miR-513-5p, ocu-miR-520e, ocu-miR-532-3p, ocu-miR-532-5p, ocu-miR-539-3p, ocu-miR-539-5p, ocu-miR-541-3p, ocu-miR-541-5p, ocu-miR-542-3p, ocu-miR-542-5p, ocu-miR-545-3p, ocu-miR-545-5p, ocu-miR-551b-3p, ocu-miR-551b-5p, ocu-miR-574-3p, ocu-miR-574-5p, ocu-miR-582-3p, ocu-miR-582-5p, ocu-miR-590-3p, ocu-miR-590-5p, ocu-miR-599-3p, ocu-miR-599-5p, ocu-miR-628-3p, ocu-miR-628-5p, ocu-miR-652-3p, ocu-miR-652-5p, ocu-miR-6529-3p, ocu-miR-6529-5p, ocu-miR-653-3p, ocu-miR-653-5p, ocu-miR-654-3p, ocu-miR-654-5p, ocu-miR-655-3p, ocu-miR-655-5p, ocu-miR-656-3p, ocu-miR-656-5p, ocu-miR-660-3p, ocu-miR-660-5p, ocu-miR-670-3p, ocu-miR-670-5p, ocu-miR-671-3p, ocu-miR-671-5p, ocu-miR-672-3p, ocu-miR-672-5p, ocu-miR-676-3p, ocu-miR-676-5p, ocu-miR-708-3p, ocu-miR-708-5p, ocu-miR-7180-3p, ocu-miR-7180-5p, ocu-miR-744-3p, ocu-miR-744-5p, ocu-miR-758-3p, ocu-miR-758-5p, ocu-miR-760-3p, ocu-miR-760-5p, ocu-miR-7a-3p, ocu-miR-7a-5p, ocu-miR-802-3p, ocu-miR-802-5p, ocu-miR-872-3p, ocu-miR-872-5p, ocu-miR-873-3p, ocu-miR-873-5p, ocu-miR-874-3p, ocu-miR-874-5p, ocu-miR-875-3p, ocu-miR-875-5p, ocu-miR-885-3p, ocu-miR-885-5p, ocu-miR-889-3p, ocu-miR-889-5p, ocu-miR-9-3p, ocu-miR-9-5p, ocu-miR-92a-2-5p, ocu-miR-92a-3p, ocu-miR-92a-5p, ocu-miR-93-3p, ocu-miR-93-5p, ocu-miR-96-3p, ocu-miR-96-5p, ocu-miR-98-3p, ocu-miR-98-5p, ocu-miR-9851-3p, ocu-miR-9851-5p, ocu-miR-99a-3p, ocu-miR-99a-5p, odi-let-7a, odi-let-7b, odi-let-7c, odi-let-7d, odi-miR-124a, odi-miR-124b, odi-miR-1468, odi-miR-1469, odi-miR-1470, odi-miR-1471, odi-miR-1472, odi-miR-1473, odi-miR-1474, odi-miR-1475, odi-miR-1476-3p, odi-miR-1476-5p, odi-miR-1477, odi-miR-1478, odi-miR-1479, odi-miR-1480, odi-miR-1481, odi-miR-1482, odi-miR-1483-3p, odi-miR-1483-5p, odi-miR-1484, odi-miR-1485, odi-miR-1486, odi-miR-1487, odi-miR-1489, odi-miR-1490a, odi-miR-1490b, odi-miR-1491, odi-miR-1492, odi-miR-1493, odi-miR-1494, odi-miR-1495, odi-miR-1496, odi-miR-1497a, odi-miR-1497b, odi-miR-1497c, odi-miR-1497d, odi-miR-1497e, odi-miR-1497f, odi-miR-1497g, odi-miR-1497h, odi-miR-1498, odi-miR-1499, odi-miR-1500, odi-miR-1501, odi-miR-1502, odi-miR-1503, odi-miR-1504, odi-miR-1505, odi-miR-1506, odi-miR-1a, odi-miR-1b, odi-miR-1c, odi-miR-219, odi-miR-281, odi-miR-31, odi-miR-7, odi-miR-92a, odi-miR-92b, oga-let-7a, oga-let-7b, oga-let-7c, oga-let-7e, oga-let-7f, oga-let-7g, oga-let-7i, oga-miR-10, oga-miR-100, oga-miR-101, oga-miR-103a, oga-miR-103b, oga-miR-106b, oga-miR-107, oga-miR-10a, oga-miR-1247, oga-miR-125a, oga-miR-125b, oga-miR-127, oga-miR-128, oga-miR-1296, oga-miR-130a, oga-miR-132, oga-miR-136, oga-miR-138, oga-miR-1388, oga-miR-140, oga-miR-143, oga-miR-145, oga-miR-148a, oga-miR-148b, oga-miR-149, oga-miR-151, oga-miR-152, oga-miR-155, oga-miR-15a, oga-miR-15b, oga-miR-16, oga-miR-17, oga-miR-18, oga-miR-181a, oga-miR-181b, oga-miR-181c, oga-miR-181d, oga-miR-181e, oga-miR-182, oga-miR-184, oga-miR-185, oga-miR-186, oga-miR-190, oga-miR-191, oga-miR-192, oga-miR-193a, oga-miR-193b, oga-miR-195, oga-miR-196, oga-miR-196a, oga-miR-196b, oga-miR-197, oga-miR-199, oga-miR-19a, oga-miR-19b, oga-miR-21, oga-miR-210, oga-miR-212, oga-miR-214, oga-miR-218a, oga-miR-22, oga-miR-221, oga-miR-222, oga-miR-224, oga-miR-23a, oga-miR-23b, oga-miR-24, oga-miR-25, oga-miR-26a, oga-miR-26b, oga-miR-27a, oga-miR-27b, oga-miR-28a, oga-miR-28b, oga-miR-29a, oga-miR-29b, oga-miR-301a, oga-miR-30a, oga-miR-30b, oga-miR-30c, oga-miR-30d, oga-miR-30e, oga-miR-31, oga-miR-320a, oga-miR-330, oga-miR-340, oga-miR-342, oga-miR-34a, oga-miR-34b, oga-miR-34c, oga-miR-361, oga-miR-365, oga-miR-374a, oga-miR-378, oga-miR-381, oga-miR-409, oga-miR-410, oga-miR-411a, oga-miR-421, oga-miR-423, oga-miR-455, oga-miR-486, oga-miR-493, oga-miR-497, oga-miR-500, oga-miR-502b, oga-miR-505, oga-miR-532, oga-miR-574, oga-miR-582, oga-miR-615, oga-miR-6529, oga-miR-660, oga-miR-760, oga-miR-872, oga-miR-92a, oga-miR-93, oga-miR-98, oga-miR-99a, oga-miR-99b, oha-let-7a, oha-let-7a-2-3p, oha-let-7a-3-3p, oha-let-7a-5p, oha-let-7b-3p, oha-let-7b-5p, oha-let-7c-1-3p, oha-let-7c-2-3p, oha-let-7c-3-3p, oha-let-7c-5p, oha-let-7d-3p, oha-let-7d-5p, oha-let-7e-3p, oha-let-7e-5p, oha-let-7f-1-3p, oha-let-7f-2-3p, oha-let-7f-5p, oha-let-7g-3p, oha-let-7g-5p, oha-let-7i-3p, oha-let-7i-5p, oha-miR-100-3p, oha-miR-100-5p, oha-miR-101a-3p, oha-miR-101a-5p, oha-miR-101b-3p, oha-miR-101b-5p, oha-miR-103a-3p, oha-miR-103a-5p, oha-miR-103b-3p, oha-miR-103b-5p, oha-miR-107-3p, oha-miR-107-5p, oha-miR-10a-3p, oha-miR-10a-5p, oha-miR-10b-3p, oha-miR-10b-5p, oha-miR-10c-3p, oha-miR-10c-5p, oha-miR-122-3p, oha-miR-122-5p, oha-miR-124, oha-miR-124-3p, oha-miR-124-4-3p, oha-miR-124-5p, oha-miR-125a-3p, oha-miR-125a-5p, oha-miR-125b-3p, oha-miR-125b-5p, oha-miR-126-3p, oha-miR-126-5p, oha-miR-128-3p, oha-miR-128-5p, oha-miR-129a-3p, oha-miR-129a-5p, oha-miR-129b, oha-miR-129b-3p, oha-miR-129b-5p, oha-miR-1306-3p, oha-miR-1306-5p, oha-miR-130a-3p, oha-miR-130a-5p, oha-miR-130b-3p, oha-miR-130b-5p, oha-miR-130c-3p, oha-miR-130c-5p, oha-miR-130d, oha-miR-132-3p, oha-miR-132-5p, oha-miR-1329-3p, oha-miR-1329-5p, oha-miR-133a-1-5p, oha-miR-133a-2-5p, oha-miR-133a-3-5p, oha-miR-133a-3p, oha-miR-133b-3p, oha-miR-133b-5p, oha-miR-135-1-3p, oha-miR-135-2-3p, oha-miR-135-3-3p, oha-miR-135-5p, oha-miR-137-3p, oha-miR-137-5p, oha-miR-138-1-3p, oha-miR-138-2-3p, oha-miR-138-5p, oha-miR-1388-3p, oha-miR-1388-5p, oha-miR-139-3p, oha-miR-139-5p, oha-miR-1397-3p, oha-miR-1397-5p, oha-miR-140-3p, oha-miR-140-5p, oha-miR-142-3p, oha-miR-142-5p, oha-miR-143-3p, oha-miR-143-5p, oha-miR-144-3p, oha-miR-144-5p, oha-miR-145-3p, oha-miR-145-5p, oha-miR-146b-3p, oha-miR-146b-5p, oha-miR-147-3p, oha-miR-147-5p, oha-miR-148a-3p, oha-miR-148a-5p, oha-miR-148b, oha-miR-150, oha-miR-153-1-5p, oha-miR-153-2-5p, oha-miR-153-3p, oha-miR-15a-3p, oha-miR-15a-5p, oha-miR-15b-3p, oha-miR-15b-5p, oha-miR-1641, oha-miR-1677b-3p, oha-miR-1677b-5p, oha-miR-16a-3p, oha-miR-16a-5p, oha-miR-16b-3p, oha-miR-16b-5p, oha-miR-17-3p, oha-miR-17-5p, oha-miR-1788-3p, oha-miR-1788-5p, oha-miR-1805-3p, oha-miR-1805-5p, oha-miR-181a-1-3p, oha-miR-181a-2-3p, oha-miR-181a-5p, oha-miR-181b-3p, oha-miR-181b-5p, oha-miR-181c-3p, oha-miR-181c-5p, oha-miR-182-3p, oha-miR-182-5p, oha-miR-183-3p, oha-miR-183-5p, oha-miR-18a-3p, oha-miR-18a-5p, oha-miR-18b-3p, oha-miR-18b-5p, oha-miR-1905, oha-miR-190a-3p, oha-miR-190a-5p, oha-miR-190b-3p, oha-miR-190b-5p, oha-miR-191-3p, oha-miR-191-5p, oha-miR-193-3p, oha-miR-193-5p, oha-miR-194-3p, oha-miR-194-5p, oha-miR-196a-3p, oha-miR-196a-5p, oha-miR-196b-3p, oha-miR-196b-5p, oha-miR-196c-3p, oha-miR-196c-5p, oha-miR-199a-3p, oha-miR-199a-5p, oha-miR-199b-3p, oha-miR-199b-5p, oha-miR-199c-3p, oha-miR-199c-5p, oha-miR-19a-3p, oha-miR-19a-5p, oha-miR-19b-3p, oha-miR-19b-5p, oha-miR-1a-3p, oha-miR-1a-5p, oha-miR-1b-3p, oha-miR-1b-5p, oha-miR-1c-3p, oha-miR-1c-5p, oha-miR-1d-3p, oha-miR-1d-5p, oha-miR-200a, oha-miR-202-3p, oha-miR-202-5p, oha-miR-203-3p, oha-miR-203-5p, oha-miR-204-1-3p, oha-miR-204-2-3p, oha-miR-204-5p, oha-miR-205a-3p, oha-miR-205a-5p, oha-miR-205b-3p, oha-miR-205b-5p, oha-miR-206, oha-miR-208-3p, oha-miR-208-5p, oha-miR-20a-3p, oha-miR-20a-5p, oha-miR-20b-3p, oha-miR-20b-5p, oha-miR-21-3p, oha-miR-21-5p, oha-miR-210-3p, oha-miR-210-5p, oha-miR-212-3p, oha-miR-212-5p, oha-miR-215-3p, oha-miR-215-5p, oha-miR-216-3p, oha-miR-216-5p, oha-miR-217-5p, oha-miR-218-1-3p, oha-miR-218-2-3p, oha-miR-218-5p, oha-miR-2188-3p, oha-miR-2188-5p, oha-miR-219-1-3p, oha-miR-219-2-3p, oha-miR-219-5p, oha-miR-221-3p, oha-miR-221-5p, oha-miR-222a-3p, oha-miR-222a-5p, oha-miR-222b-3p, oha-miR-222b-5p, oha-miR-223-3p, oha-miR-223-5p, oha-miR-22a, oha-miR-22b, oha-miR-23a-3p, oha-miR-23a-5p, oha-miR-23b-3p, oha-miR-23b-5p, oha-miR-24-1-5p, oha-miR-24-2-5p, oha-miR-24-3p, oha-miR-26-1-3p, oha-miR-26-2-3p, oha-miR-26-3-3p, oha-miR-26-5p, oha-miR-27a-3p, oha-miR-27a-5p, oha-miR-27b-3p, oha-miR-27b-5p, oha-miR-2970-3p, oha-miR-2970-5p, oha-miR-2985a-2-3p, oha-miR-2985a-3p, oha-miR-2985a-5p, oha-miR-2985b-3p, oha-miR-2985b-5p, oha-miR-29a-1-5p, oha-miR-29a-2-5p, oha-miR-29a-3p, oha-miR-29b-1-5p, oha-miR-29b-2-5p, oha-miR-29b-3p, oha-miR-301a-3p, oha-miR-301a-5p, oha-miR-301b-3p, oha-miR-301b-5p, oha-miR-30a-3p, oha-miR-30a-5p, oha-miR-30b-3p, oha-miR-30b-5p, oha-miR-30c-1-3p, oha-miR-30c-2-3p, oha-miR-30c-5p, oha-miR-30d-3p, oha-miR-30d-5p, oha-miR-30e-3p, oha-miR-30e-5p, oha-miR-31-3p, oha-miR-31-5p, oha-miR-3120, oha-miR-32-3p, oha-miR-32-5p, oha-miR-33-3p, oha-miR-33-5p, oha-miR-338-3p, oha-miR-338-5p, oha-miR-33a-3p, oha-miR-33a-5p, oha-miR-34a-3p, oha-miR-34a-5p, oha-miR-34c-3p, oha-miR-34c-5p, oha-miR-3618, oha-miR-363-3p, oha-miR-363-5p, oha-miR-365a-1-5p, oha-miR-365a-2-5p, oha-miR-365a-3p, oha-miR-375-3p, oha-miR-375-5p, oha-miR-383-3p, oha-miR-383-5p, oha-miR-425-3p, oha-miR-425-5p, oha-miR-429-3p, oha-miR-429-5p, oha-miR-449a-3p, oha-miR-449a-5p, oha-miR-449c-3p, oha-miR-449c-5p, oha-miR-451-??, oha-miR-451-3p, oha-miR-455-3p, oha-miR-455-5p, oha-miR-456, oha-miR-458-3p, oha-miR-458-5p, oha-miR-490-3p, oha-miR-490-5p, oha-miR-499-3p, oha-miR-499-5p, oha-miR-5391, oha-miR-5470, oha-miR-551b-3p, oha-miR-551b-5p, oha-miR-599, oha-miR-7-1-3p, oha-miR-7-2-3p, oha-miR-7-3-3p, oha-miR-7-5p, oha-miR-737-3p, oha-miR-737-5p, oha-miR-875-3p, oha-miR-875-5p, oha-miR-9-1-5p, oha-miR-9-2-5p, oha-miR-9-3-5p, oha-miR-9-3p, oha-miR-9-4-5p, oha-miR-92a, oha-miR-92a-3p, oha-miR-92a-5p, oha-miR-96, oha-miR-98-3p, oha-miR-98-5p, oha-miR-99a-3p, oha-miR-99a-5p, oha-miR-99b-3p, oha-miR-99b-5p, ola-let-7a, ola-let-7a-3p, ola-let-7a-5p, ola-let-7b, ola-let-7c, ola-let-7e, ola-let-7g, ola-miR-1-3p, ola-miR-1-5p, ola-miR-100, ola-miR-101a-3p, ola-miR-101a-5p, ola-miR-101b-3p, ola-miR-101b-5p, ola-miR-103, ola-miR-106a, ola-miR-107, ola-miR-10b, ola-miR-10d, ola-miR-122, ola-miR-124-3p, ola-miR-124-5p, ola-miR-125a-3p, ola-miR-125a-5p, ola-miR-125b, ola-miR-125c, ola-miR-126-3p, ola-miR-126-5p, ola-miR-128, ola-miR-1306, ola-miR-130c, ola-miR-132, ola-miR-133-3p, ola-miR-133-5p, ola-miR-135a, ola-miR-135b, ola-miR-137, ola-miR-138, ola-miR-1388-3p, ola-miR-1388-5p, ola-miR-139, ola-miR-140-3p, ola-miR-140-5p, ola-miR-142, ola-miR-143, ola-miR-144, ola-miR-145, ola-miR-146a-5p, ola-miR-148, ola-miR-150, ola-miR-152, ola-miR-15a, ola-miR-16, ola-miR-17, ola-miR-1788, ola-miR-18, ola-miR-181a-3p, ola-miR-181a-5p, ola-miR-181b-3p, ola-miR-181b-5p, ola-miR-182, ola-miR-183-3p, ola-miR-183-5p, ola-miR-184-3p, ola-miR-184-5p, ola-miR-187, ola-miR-190b, ola-miR-192-3p, ola-miR-192-5p, ola-miR-193a, ola-miR-194-3p, ola-miR-194-5p, ola-miR-196a, ola-miR-199a-3p, ola-miR-199a-5p, ola-miR-19a, ola-miR-19b, ola-miR-19d, ola-miR-200a, ola-miR-200b, ola-miR-203, ola-miR-204, ola-miR-205, ola-miR-206, ola-miR-20a, ola-miR-21, ola-miR-210-3p, ola-miR-210-5p, ola-miR-214, ola-miR-218a, ola-miR-218b, ola-miR-22, ola-miR-221, ola-miR-222, ola-miR-223, ola-miR-23a, ola-miR-23b, ola-miR-24a, ola-miR-24b-3p, ola-miR-24b-5p, ola-miR-24c, ola-miR-26, ola-miR-27a, ola-miR-27b-5p, ola-miR-27c-3p, ola-miR-27c-5p, ola-miR-27d-3p, ola-miR-29a, ola-miR-29b, ola-miR-29c, ola-miR-301a-3p, ola-miR-301a-5p, ola-miR-301b-3p, ola-miR-301b-5p, ola-miR-30a-3p, ola-miR-30a-5p, ola-miR-30c, ola-miR-30d-3p, ola-miR-30d-5p, ola-miR-30e, ola-miR-31, ola-miR-33, ola-miR-338-3p, ola-miR-338-5p, ola-miR-430a, ola-miR-430b, ola-miR-430c, ola-miR-430d, ola-miR-455-3p, ola-miR-455-5p, ola-miR-458, ola-miR-459-3p, ola-miR-459-5p, ola-miR-460-3p, ola-miR-460-5p, ola-miR-462, ola-miR-499, ola-miR-7, ola-miR-729, ola-miR-731, ola-miR-92a, ola-miR-92b, ola-miR-99, ola-miR-9a-3p, ola-miR-9a-5p, ola-miR-9b-5p, oni-let-7a, oni-let-7b, oni-let-7c, oni-let-7d, oni-let-7e, oni-let-7f, oni-let-7g, oni-let-7h, oni-let-7i, oni-miR-1, oni-miR-100, oni-miR-101a, oni-miR-101b, oni-miR-103, oni-miR-10544, oni-miR-10545, oni-miR-10547a, oni-miR-10547b, oni-miR-10547c, oni-miR-10547d, oni-miR-10548a, oni-miR-10548b, oni-miR-10548c, oni-miR-10548d, oni-miR-10549, oni-miR-10551, oni-miR-10552, oni-miR-10553, oni-miR-10555, oni-miR-10556a, oni-miR-10556b, oni-miR-10558, oni-miR-10559, oni-miR-10562, oni-miR-10563, oni-miR-10569, oni-miR-10573a, oni-miR-10573b, oni-miR-10573c, oni-miR-10573d, oni-miR-10574, oni-miR-10575, oni-miR-10576, oni-miR-10577, oni-miR-10578, oni-miR-10579, oni-miR-10580, oni-miR-10581a, oni-miR-10581b, oni-miR-10581c, oni-miR-10581d, oni-miR-10582, oni-miR-10583, oni-miR-10584, oni-miR-10585, oni-miR-10586, oni-miR-10587, oni-miR-10588, oni-miR-10589, oni-miR-10590, oni-miR-10591, oni-miR-10592, oni-miR-10593, oni-miR-10594a, oni-miR-10594b, oni-miR-10594c, oni-miR-10595, oni-miR-10596-3p, oni-miR-10596-5p, oni-miR-10597a, oni-miR-10597b, oni-miR-10597c, oni-miR-10597d, oni-miR-10598, oni-miR-10599, oni-miR-106, oni-miR-10600, oni-miR-10601, oni-miR-10602, oni-miR-10603a, oni-miR-10603b, oni-miR-10603c, oni-miR-10604, oni-miR-10605, oni-miR-10606, oni-miR-10607, oni-miR-10608a, oni-miR-10608b, oni-miR-10609, oni-miR-10610a, oni-miR-10610b, oni-miR-10610c, oni-miR-10611, oni-miR-10612, oni-miR-10613, oni-miR-10614, oni-miR-10615a, oni-miR-10615b, oni-miR-10616a, oni-miR-10616b, oni-miR-10617, oni-miR-10618, oni-miR-10619, oni-miR-10620, oni-miR-10621a, oni-miR-10621b, oni-miR-10622, oni-miR-10623, oni-miR-10624, oni-miR-10625, oni-miR-10626, oni-miR-10627, oni-miR-10628, oni-miR-10629, oni-miR-10630, oni-miR-10631, oni-miR-10632, oni-miR-10633, oni-miR-10634, oni-miR-10635, oni-miR-10636, oni-miR-10637, oni-miR-10638, oni-miR-10639, oni-miR-10640, oni-miR-10641, oni-miR-10642, oni-miR-10643, oni-miR-10644, oni-miR-10645, oni-miR-10646a, oni-miR-10646b, oni-miR-10647, oni-miR-10648, oni-miR-10649, oni-miR-10650, oni-miR-10651, oni-miR-10652, oni-miR-10653, oni-miR-10654, oni-miR-10655, oni-miR-10656, oni-miR-10657, oni-miR-10658, oni-miR-10659, oni-miR-10660, oni-miR-10661, oni-miR-10662, oni-miR-10663, oni-miR-10664, oni-miR-10665, oni-miR-10666, oni-miR-10667, oni-miR-10668, oni-miR-10669, oni-miR-10670, oni-miR-10671, oni-miR-10672, oni-miR-10673, oni-miR-10674, oni-miR-10675, oni-miR-10676, oni-miR-10677, oni-miR-10678, oni-miR-10679, oni-miR-10680, oni-miR-10681, oni-miR-10682, oni-miR-10683, oni-miR-10684, oni-miR-10685, oni-miR-10686, oni-miR-10687, oni-miR-10688, oni-miR-10689, oni-miR-10690, oni-miR-10691, oni-miR-10692, oni-miR-10693, oni-miR-10694, oni-miR-10695, oni-miR-10696, oni-miR-10697a, oni-miR-10697b, oni-miR-10698, oni-miR-10699, oni-miR-107, oni-miR-10700, oni-miR-10701, oni-miR-10702, oni-miR-10703, oni-miR-10704, oni-miR-10705, oni-miR-10706, oni-miR-10707, oni-miR-10708, oni-miR-10709, oni-miR-10710, oni-miR-10711, oni-miR-10712, oni-miR-10713, oni-miR-10714, oni-miR-10715, oni-miR-10716, oni-miR-10717, oni-miR-10718, oni-miR-10719, oni-miR-10720, oni-miR-10721, oni-miR-10722, oni-miR-10723, oni-miR-10724, oni-miR-10725, oni-miR-10726, oni-miR-10727, oni-miR-10728, oni-miR-10729, oni-miR-10730, oni-miR-10731, oni-miR-10732, oni-miR-10733, oni-miR-10734, oni-miR-10735, oni-miR-10736, oni-miR-10737, oni-miR-10738, oni-miR-10739, oni-miR-10740, oni-miR-10741, oni-miR-10742, oni-miR-10743, oni-miR-10744, oni-miR-10745, oni-miR-10746, oni-miR-10747, oni-miR-10748, oni-miR-10749, oni-miR-10750, oni-miR-10751, oni-miR-10752, oni-miR-10753, oni-miR-10754, oni-miR-10755, oni-miR-10756, oni-miR-10757, oni-miR-10758, oni-miR-10759, oni-miR-10760, oni-miR-10761, oni-miR-10762, oni-miR-10763, oni-miR-10764, oni-miR-10765, oni-miR-10766, oni-miR-10767, oni-miR-10768, oni-miR-10769, oni-miR-10770, oni-miR-10771, oni-miR-10772, oni-miR-10773, oni-miR-10774, oni-miR-10775, oni-miR-10776, oni-miR-10777, oni-miR-10778, oni-miR-10779, oni-miR-10780, oni-miR-10781, oni-miR-10782, oni-miR-10783, oni-miR-10784, oni-miR-10785, oni-miR-10786, oni-miR-10787, oni-miR-10788, oni-miR-10789, oni-miR-10790, oni-miR-10791, oni-miR-10792, oni-miR-10793, oni-miR-10794, oni-miR-10795, oni-miR-10796, oni-miR-10797, oni-miR-10798, oni-miR-10799, oni-miR-10800, oni-miR-10801, oni-miR-10802, oni-miR-10803, oni-miR-10804, oni-miR-10805, oni-miR-10806, oni-miR-10807, oni-miR-10808, oni-miR-10809, oni-miR-10810, oni-miR-10811, oni-miR-10812, oni-miR-10813, oni-miR-10814, oni-miR-10815, oni-miR-10816, oni-miR-10817, oni-miR-10818, oni-miR-10819, oni-miR-10820, oni-miR-10821, oni-miR-10822, oni-miR-10823, oni-miR-10824, oni-miR-10825, oni-miR-10826, oni-miR-10827, oni-miR-10828, oni-miR-10829, oni-miR-10830, oni-miR-10831a, oni-miR-10831b, oni-miR-10832, oni-miR-10833, oni-miR-10834, oni-miR-10835, oni-miR-10836, oni-miR-10837, oni-miR-10838, oni-miR-10839, oni-miR-10840, oni-miR-10841, oni-miR-10842, oni-miR-10843, oni-miR-10844, oni-miR-10845, oni-miR-10846, oni-miR-10847, oni-miR-10848, oni-miR-10849, oni-miR-10850, oni-miR-10851, oni-miR-10852, oni-miR-10853, oni-miR-10854, oni-miR-10855, oni-miR-10856, oni-miR-10857, oni-miR-10858, oni-miR-10859, oni-miR-10860, oni-miR-10861, oni-miR-10862, oni-miR-10863, oni-miR-10864, oni-miR-10865, oni-miR-10866, oni-miR-10867, oni-miR-10868, oni-miR-10869, oni-miR-10870, oni-miR-10871, oni-miR-10872, oni-miR-10873, oni-miR-10874, oni-miR-10875, oni-miR-10876, oni-miR-10877, oni-miR-10878, oni-miR-10879, oni-miR-10880, oni-miR-10881, oni-miR-10882, oni-miR-10883, oni-miR-10884, oni-miR-10885, oni-miR-10886, oni-miR-10887, oni-miR-10888, oni-miR-10889, oni-miR-10890, oni-miR-10891, oni-miR-10892, oni-miR-10893, oni-miR-10894, oni-miR-10895, oni-miR-10896, oni-miR-10897, oni-miR-10898, oni-miR-10899, oni-miR-10900a, oni-miR-10900b, oni-miR-10901, oni-miR-10902, oni-miR-10903, oni-miR-10904, oni-miR-10905, oni-miR-10906, oni-miR-10907, oni-miR-10908, oni-miR-10909, oni-miR-10910, oni-miR-10912, oni-miR-10913, oni-miR-10914, oni-miR-10915, oni-miR-10916, oni-miR-10917, oni-miR-10918, oni-miR-10919, oni-miR-10920, oni-miR-10921, oni-miR-10922, oni-miR-10923, oni-miR-10924, oni-miR-10925, oni-miR-10926, oni-miR-10927, oni-miR-10928, oni-miR-10929, oni-miR-10930, oni-miR-10931, oni-miR-10932, oni-miR-10933, oni-miR-10934, oni-miR-10935, oni-miR-10936, oni-miR-10937, oni-miR-10938, oni-miR-10939, oni-miR-10940, oni-miR-10941, oni-miR-10942, oni-miR-10943, oni-miR-10944, oni-miR-10946, oni-miR-10947, oni-miR-10948, oni-miR-10949, oni-miR-10950, oni-miR-10951, oni-miR-10952, oni-miR-10953, oni-miR-10954, oni-miR-10955, oni-miR-10956, oni-miR-10957, oni-miR-10958, oni-miR-10959, oni-miR-10960, oni-miR-10961, oni-miR-10962, oni-miR-10963, oni-miR-10964, oni-miR-10965, oni-miR-10966, oni-miR-10967, oni-miR-10968, oni-miR-10969, oni-miR-10970, oni-miR-10971, oni-miR-10972, oni-miR-10973, oni-miR-10974, oni-miR-10975, oni-miR-10976, oni-miR-10977, oni-miR-10a, oni-miR-10b, oni-miR-10c, oni-miR-10d, oni-miR-122, oni-miR-124a, oni-miR-124b, oni-miR-125a, oni-miR-125b, oni-miR-126, oni-miR-128, oni-miR-129, oni-miR-130a, oni-miR-130b-3p, oni-miR-130b-5p, oni-miR-132a, oni-miR-132b-3p, oni-miR-132b-5p, oni-miR-132c, oni-miR-132d, oni-miR-133a, oni-miR-133b, oni-miR-135a, oni-miR-135b, oni-miR-135c, oni-miR-135c-3p, oni-miR-135d, oni-miR-137a, oni-miR-137b, oni-miR-138, oni-miR-1388, oni-miR-139, oni-miR-140, oni-miR-142, oni-miR-142b, oni-miR-143, oni-miR-144a, oni-miR-144b, oni-miR-145, oni-miR-147, oni-miR-148-3p, oni-miR-148-5p, oni-miR-150, oni-miR-153a, oni-miR-153b, oni-miR-153c, oni-miR-153d, oni-miR-155, oni-miR-15a, oni-miR-15b, oni-miR-15c, oni-miR-16a, oni-miR-16b, oni-miR-1788, oni-miR-17a, oni-miR-17b, oni-miR-181a, oni-miR-181b, oni-miR-181c, oni-miR-181d, oni-miR-182, oni-miR-183, oni-miR-184a, oni-miR-184b, oni-miR-184c, oni-miR-184d, oni-miR-187, oni-miR-18a, oni-miR-18a-2, oni-miR-18b, oni-miR-190a, oni-miR-190b, oni-miR-192, oni-miR-193, oni-miR-194a, oni-miR-194b, oni-miR-196a, oni-miR-196b, oni-miR-199a, oni-miR-199b, oni-miR-199c, oni-miR-19a, oni-miR-19b, oni-miR-19c, oni-miR-19d, oni-miR-20, oni-miR-200a, oni-miR-200b, oni-miR-202, oni-miR-203a, oni-miR-203b, oni-miR-204a, oni-miR-204b, oni-miR-205, oni-miR-206, oni-miR-21, oni-miR-210, oni-miR-212a-3p, oni-miR-212a-5p, oni-miR-212b, oni-miR-214, oni-miR-216a, oni-miR-216b, oni-miR-217, oni-miR-2184, oni-miR-2187a, oni-miR-2187b, oni-miR-2188, oni-miR-218a, oni-miR-218b, oni-miR-219a-3p, oni-miR-219a-5p, oni-miR-219b, oni-miR-219c-3p, oni-miR-219c-5p, oni-miR-219d, oni-miR-221, oni-miR-222, oni-miR-22a, oni-miR-22b, oni-miR-22c, oni-miR-23a, oni-miR-23b, oni-miR-23c, oni-miR-23d, oni-miR-24a, oni-miR-24a-3, oni-miR-24a-4, oni-miR-24b-3p, oni-miR-24b-5p, oni-miR-25, oni-miR-26a, oni-miR-26b, oni-miR-26c, oni-miR-26d, oni-miR-27a, oni-miR-27b, oni-miR-27c, oni-miR-27d-3p, oni-miR-27d-5p, oni-miR-27e, oni-miR-27f, oni-miR-29a, oni-miR-29b, oni-miR-29c, oni-miR-29d, oni-miR-301a, oni-miR-301b, oni-miR-301c, oni-miR-30a, oni-miR-30b, oni-miR-30c, oni-miR-30d, oni-miR-31, oni-miR-3120-3p, oni-miR-3120-5p, oni-miR-338, oni-miR-33a-3p, oni-miR-33a-5p, oni-miR-33b-3p, oni-miR-33b-5p, oni-miR-34, oni-miR-34b, oni-miR-34c, oni-miR-3553, oni-miR-365, oni-miR-375, oni-miR-429b, oni-miR-430a, oni-miR-430b, oni-miR-449a, oni-miR-449b-3p, oni-miR-449b-5p, oni-miR-449c, oni-miR-451b, oni-miR-454b, oni-miR-455, oni-miR-456, oni-miR-457, oni-miR-458, oni-miR-4585, oni-miR-460, oni-miR-462, oni-miR-489, oni-miR-499, oni-miR-551, oni-miR-6960, oni-miR-7, oni-miR-7132a-3p, oni-miR-7132a-5p, oni-miR-7132b-3p, oni-miR-7132b-5p, oni-miR-7133, oni-miR-7144, oni-miR-7147, oni-miR-722, oni-miR-723a, oni-miR-723b, oni-miR-724, oni-miR-725, oni-miR-726, oni-miR-726b, oni-miR-727a, oni-miR-727b, oni-miR-728a, oni-miR-728b, oni-miR-729-3p, oni-miR-729-5p, oni-miR-730, oni-miR-731, oni-miR-734, oni-miR-736, oni-miR-7550, oni-miR-7552, oni-miR-7565, oni-miR-8159, oni-miR-8160a, oni-miR-8160b, oni-miR-92a, oni-miR-92b, oni-miR-93, oni-miR-96, oni-miR-99a, oni-miR-99b, oni-miR-9a, oni-miR-9a-3p, oni-miR-9a-5p, oni-miR-9a-7-3p, oni-miR-9b, oni-miR-9c-5p, osa-miR11336-3p, osa-miR11336-5p, osa-miR11337-3p, osa-miR11337-5p, osa-miR11338-3p, osa-miR11338-5p, osa-miR11339-3p, osa-miR11339-5p, osa-miR11340-3p, osa-miR11340-5p, osa-miR11341-3p, osa-miR11341-5p, osa-miR11342-3p, osa-miR11342-5p, osa-miR11343-3p, osa-miR11343-5p, osa-miR11344-3p, osa-miR11344-5p, osa-miR1319a, osa-miR1319b, osa-miR1320-3p, osa-miR1320-5p, osa-miR1423-3p, osa-miR1423-5p, osa-miR1424, osa-miR1425-3p, osa-miR1425-5p, osa-miR1426, osa-miR1427, osa-miR1428a-3p, osa-miR1428a-5p, osa-miR1428b, osa-miR1428c, osa-miR1428d, osa-miR1428e-3p, osa-miR1428e-5p, osa-miR1428f-5p, osa-miR1428g-5p, osa-miR1429-3p, osa-miR1429-5p, osa-miR1430, osa-miR1431, osa-miR1432-3p, osa-miR1432-5p, osa-miR1435, osa-miR1436, osa-miR1437a, osa-miR1437b-3p, osa-miR1437b-5p, osa-miR1438, osa-miR1439, osa-miR1440a, osa-miR1440b, osa-miR1441, osa-miR1442, osa-miR156a, osa-miR156b-3p, osa-miR156b-5p, osa-miR156c-3p, osa-miR156c-5p, osa-miR156d, osa-miR156e, osa-miR156f-3p, osa-miR156f-5p, osa-miR156g-3p, osa-miR156g-5p, osa-miR156 h-3p, osa-miR156 h-5p, osa-miR156i, osa-miR156j-3p, osa-miR156j-5p, osa-miR156k, osa-miR1561-3p, osa-miR1561-5p, osa-miR159a.1, osa-miR159a.2, osa-miR159b, osa-miR159c, osa-miR159d, osa-miR159e, osa-miR159f, osa-miR160a-3p, osa-miR160a-5p, osa-miR160b-3p, osa-miR160b-5p, osa-miR160c-3p, osa-miR160c-5p, osa-miR160d-3p, osa-miR160d-5p, osa-miR160e-3p, osa-miR160e-5p, osa-miR160f-3p, osa-miR160f-5p, osa-miR162a, osa-miR162b, osa-miR164a, osa-miR164b, osa-miR164c, osa-miR164d, osa-miR164e, osa-miR164f, osa-miR166a-3p, osa-miR166a-5p, osa-miR166b-3p, osa-miR166b-5p, osa-miR166c-3p, osa-miR166c-5p, osa-miR166d-3p, osa-miR166d-5p, osa-miR166e-3p, osa-miR166e-5p, osa-miR166f, osa-miR166g-3p, osa-miR166g-5p, osa-miR166 h-3p, osa-miR166 h-5p, osa-miR166i-3p, osa-miR166i-5p, osa-miR166j-3p, osa-miR166j-5p, osa-miR166k-3p, osa-miR166k-5p, osa-miR1661-3p, osa-miR1661-5p, osa-miR166m, osa-miR167a-3p, osa-miR167a-5p, osa-miR167b, osa-miR167c-3p, osa-miR167c-5p, osa-miR167d-3p, osa-miR167d-5p, osa-miR167e-3p, osa-miR167e-5p, osa-miR167f, osa-miR167g, osa-miR167h-3p, osa-miR167 h-5p, osa-miR167i-3p, osa-miR167i-5p, osa-miR167j, osa-miR168a-3p, osa-miR168a-5p, osa-miR168b, osa-miR169a, osa-miR169b, osa-miR169c, osa-miR169d, osa-miR169e, osa-miR169f.1, osa-miR169f.2, osa-miR169g, osa-miR169h, osa-miR169i-3p, osa-miR169i-5p.1, osa-miR169i-5p.2, osa-miR169j, osa-miR169k, osa-miR1691, osa-miR169m, osa-miR169n, osa-miR1690, osa-miR169p, osa-miR169q, osa-miR169r-3p, osa-miR169r-5p, osa-miR171a, osa-miR171b, osa-miR171c-3p, osa-miR171c-5p, osa-miR171d-3p, osa-miR171d-5p, osa-miR171e-3p, osa-miR171e-5p, osa-miR171f-3p, osa-miR171f-5p, osa-miR171g, osa-miR171h, osa-miR171i-3p, osa-miR171i-5p, osa-miR172a, osa-miR172b, osa-miR172c, osa-miR172d-3p, osa-miR172d-5p, osa-miR1846a-3p, osa-miR1846a-5p, osa-miR1846b-3p, osa-miR1846b-5p, osa-miR1846c-3p, osa-miR1846c-5p, osa-miR1846d-3p, osa-miR1846d-5p, osa-miR1846e, osa-miR1847.1, osa-miR1847.2, osa-miR1848, osa-miR1849, osa-miR1850.1, osa-miR1850.2, osa-miR1850.3, osa-miR1851, osa-miR1852, osa-miR1853-3p, osa-miR1853-5p, osa-miR1854-3p, osa-miR1854-5p, osa-miR1855, osa-miR1856, osa-miR1857-3p, osa-miR1857-5p, osa-miR1858a, osa-miR1858b, osa-miR1859, osa-miR1860-3p, osa-miR1860-5p, osa-miR1861a, osa-miR1861b, osa-miR1861c, osa-miR1861d, osa-miR1861e, osa-miR1861f, osa-miR1861g, osa-miR1861h, osa-miR1861i, osa-miR1861j, osa-miR1861k, osa-miR18611, osa-miR1861m, osa-miR1861n, osa-miR1861o, osa-miR1862a, osa-miR1862b, osa-miR1862c, osa-miR1862d, osa-miR1862e, osa-miR1862f, osa-miR1862g, osa-miR1863a, osa-miR1863b, osa-miR1863b.2, osa-miR1863c, osa-miR1864, osa-miR1865-3p, osa-miR1865-5p, osa-miR1866-3p, osa-miR1866-5p, osa-miR1868, osa-miR1869, osa-miR1870-3p, osa-miR1870-5p, osa-miR1871, osa-miR1872, osa-miR1873, osa-miR1874-3p, osa-miR1874-5p, osa-miR1875, osa-miR1876, osa-miR1877, osa-miR1878, osa-miR1879, osa-miR1880, osa-miR1881, osa-miR1882a, osa-miR1882b, osa-miR1882c, osa-miR1882d, osa-miR1882e-3p, osa-miR1882e-5p, osa-miR1882f, osa-miR1882g, osa-miR1882h, osa-miR1883a, osa-miR1883b, osa-miR2055, osa-miR2090, osa-miR2091-3p, osa-miR2091-5p, osa-miR2092-3p, osa-miR2092-5p, osa-miR2093-3p, osa-miR2093-5p, osa-miR2094-3p, osa-miR2094-5p, osa-miR2095-3p, osa-miR2095-5p, osa-miR2096-3p, osa-miR2096-5p, osa-miR2097-3p, osa-miR2097-5p, osa-miR2098-3p, osa-miR2098-5p, osa-miR2099-3p, osa-miR2099-5p, osa-miR2100-3p, osa-miR2100-5p, osa-miR2101-3p, osa-miR2101-5p, osa-miR2102-3p, osa-miR2102-5p, osa-miR2103, osa-miR2104, osa-miR2105, osa-miR2106, osa-miR2118a, osa-miR2118b, osa-miR2118c, osa-miR2118d, osa-miR2118e, osa-miR2118f, osa-miR2118g, osa-miR2118h, osa-miR2118i, osa-miR2118j, osa-miR2118k, osa-miR21181, osa-miR2118m, osa-miR2118n, osa-miR21180, osa-miR2118p, osa-miR2118q, osa-miR2118r, osa-miR2120, osa-miR2120b-3p, osa-miR2120b-5p, osa-miR2121a, osa-miR2121b, osa-miR2122, osa-miR2275a, osa-miR2275b, osa-miR2275c, osa-miR2275d, osa-miR2863a, osa-miR2863b, osa-miR2863c, osa-miR2864.1, osa-miR2864.2, osa-miR2865, osa-miR2866, osa-miR2867-3p, osa-miR2867-5p, osa-miR2868, osa-miR2869, osa-miR2870, osa-miR2871a-3p, osa-miR2871a-5p, osa-miR2871b-3p, osa-miR2871b-5p, osa-miR2872, osa-miR2873a, osa-miR2873b, osa-miR2873c, osa-miR2874, osa-miR2875, osa-miR2876-3p, osa-miR2876-5p, osa-miR2877, osa-miR2878-3p, osa-miR2878-5p, osa-miR2879, osa-miR2880, osa-miR2905, osa-miR2907a, osa-miR2907b, osa-miR2907c, osa-miR2907d, osa-miR2918, osa-miR2919, osa-miR2920, osa-miR2921, osa-miR2922, osa-miR2923, osa-miR2924, osa-miR2925, osa-miR2926, osa-miR2927, osa-miR2928, osa-miR2929, osa-miR2930, osa-miR2931, osa-miR2932, osa-miR319a-3p, osa-miR319a-3p.2-3p, osa-miR319a-5p, osa-miR319b, osa-miR390-3p, osa-miR390-5p, osa-miR393a, osa-miR393b-3p, osa-miR393b-5p, osa-miR394, osa-miR395a, osa-miR395b, osa-miR395c, osa-miR395d, osa-miR395e, osa-miR395f, osa-miR395g, osa-miR395h, osa-miR395i, osa-miR395j, osa-miR395k, osa-miR3951, osa-miR395m, osa-miR395n, osa-miR3950, osa-miR395p, osa-miR395q, osa-miR395r, osa-miR395s, osa-miR395t, osa-miR395u, osa-miR395v, osa-miR395w, osa-miR395x, osa-miR395y, osa-miR396a-3p, osa-miR396a-5p, osa-miR396b-3p, osa-miR396b-5p, osa-miR396c-3p, osa-miR396c-5p, osa-miR396d, osa-miR396e-3p, osa-miR396e-5p, osa-miR396f-3p, osa-miR396f-5p, osa-miR396g, osa-miR396h, osa-miR3979-3p, osa-miR3979-5p, osa-miR397a, osa-miR397b, osa-miR3980a-3p, osa-miR3980a-5p, osa-miR3980b-3p, osa-miR3980b-5p, osa-miR3981-3p, osa-miR3981-5p, osa-miR3982-3p, osa-miR3982-5p, osa-miR398a, osa-miR398b, osa-miR399a, osa-miR399b, osa-miR399c, osa-miR399d, osa-miR399e, osa-miR399f, osa-miR399g, osa-miR399h, osa-miR399i, osa-miR399j, osa-miR399k, osa-miR408-3p, osa-miR408-5p, osa-miR413, osa-miR414, osa-miR415, osa-miR416, osa-miR417, osa-miR418, osa-miR419, osa-miR426, osa-miR435, osa-miR437, osa-miR438, osa-miR439a, osa-miR439b, osa-miR439c, osa-miR439d, osa-miR439e, osa-miR439f, osa-miR439g, osa-miR439h, osa-miR439i, osa-miR440, osa-miR443, osa-miR444a-3p.1, osa-miR444a-3p.2, osa-miR444a-5p, osa-miR444b.1, osa-miR444b.2, osa-miR444c.1, osa-miR444c.2, osa-miR444d.1, osa-miR 444d.2, osa-miR444d.3, osa-miR444e, osa-miR444f, osa-miR5071, osa-miR5072, osa-miR5073, osa-miR5074, osa-miR5075, osa-miR5076, osa-miR5077, osa-miR5078, osa-miR5079a, osa-miR5079b, osa-miR5080, osa-miR5081, osa-miR5082, osa-miR5083, osa-miR5143a, osa-miR5143b, osa-miR5144-3p, osa-miR5144-5p, osa-miR5145, osa-miR5146, osa-miR5147, osa-miR5148a, osa-miR5148b, osa-miR5148c, osa-miR5149, osa-miR5150-3p, osa-miR5150-5p, osa-miR5151, osa-miR5152-3p, osa-miR5152-5p, osa-miR5153, osa-miR5154, osa-miR5155, osa-miR5156, osa-miR5157a-3p, osa-miR5157a-5p, osa-miR5157b-3p, osa-miR5157b-5p, osa-miR5158, osa-miR5159, osa-miR5160, osa-miR5161, osa-miR5162, osa-miR5179, osa-miR528-3p, osa-miR528-5p, osa-miR529a, osa-miR529b, osa-miR530-3p, osa-miR530-5p, osa-miR531a, osa-miR531b, osa-miR531c, osa-miR5337a, osa-miR5337b, osa-miR5338, osa-miR5339, osa-miR5340, osa-miR535-3p, osa-miR535-5p, osa-miR5484, osa-miR5485, osa-miR5486, osa-miR5487, osa-miR5488, osa-miR5489, osa-miR5490, osa-miR5491, osa-miR5492, osa-miR5493, osa-miR5494, osa-miR5495, osa-miR5496, osa-miR5497, osa-miR5498, osa-miR5499, osa-miR5500, osa-miR5501, osa-miR5502, osa-miR5503, osa-miR5504, osa-miR5505, osa-miR5506, osa-miR5507, osa-miR5508, osa-miR5509, osa-miR5510, osa-miR5511, osa-miR5512a, osa-miR5512b, osa-miR5513, osa-miR5514, osa-miR5515, osa-miR5516a, osa-miR5516b, osa-miR5517, osa-miR5518, osa-miR5519, osa-miR5521, osa-miR5522, osa-miR5523, osa-miR5524, osa-miR5525, osa-miR5526, osa-miR5527, osa-miR5528, osa-miR5529, osa-miR5530, osa-miR5531, osa-miR5532, osa-miR5533, osa-miR5534a, osa-miR5534b, osa-miR5535, osa-miR5536, osa-miR5537, osa-miR5538, osa-miR5539a, osa-miR5539b, osa-miR5540, osa-miR5541, osa-miR5542, osa-miR5543, osa-miR5544, osa-miR5788, osa-miR5789, osa-miR5790, osa-miR5791, osa-miR5792, osa-miR5793, osa-miR5794, osa-miR5795, osa-miR5796, osa-miR5797, osa-miR5798, osa-miR5799, osa-miR5800, osa-miR5801a, osa-miR5801b, osa-miR5801c-3p, osa-miR5801c-5p, osa-miR5802, osa-miR5803, osa-miR5804, osa-miR5805, osa-miR5806, osa-miR5807, osa-miR5809, osa-miR5810, osa-miR5811, osa-miR5812, osa-miR5813, osa-miR5814, osa-miR5815, osa-miR5816, osa-miR5817, osa-miR5818, osa-miR5819, osa-miR5820, osa-miR5821, osa-miR5822, osa-miR5823, osa-miR5824, osa-miR5825, osa-miR5826, osa-miR5827, osa-miR5828, osa-miR5829, osa-miR5830, osa-miR5831, osa-miR5832, osa-miR5833, osa-miR5834, osa-miR5835, osa-miR5836, osa-miR5837.1, osa-miR5837.2, osa-miR6245, osa-miR6245b-3p, osa-miR6245b-5p, osa-miR6246, osa-miR6247, osa-miR6248, osa-miR6249a, osa-miR6249b, osa-miR6250, osa-miR6251, osa-miR6252, osa-miR6253, osa-miR6254, osa-miR6255, osa-miR6256, osa-miR7692-3p, osa-miR7692-5p, osa-miR7693-3p, osa-miR7693-5p, osa-miR7694-3p, osa-miR7694-5p, osa-miR7695-3p, osa-miR7695-5p, osa-miR810a, osa-miR810b.1, osa-miR810b.2, osa-miR812a, osa-miR812b, osa-miR812c, osa-miR812d, osa-miR812e, osa-miR812f, osa-miR812g, osa-miR812h, osa-miR812i, osa-miR812j, osa-miR812k, osa-miR8121, osa-miR812m, osa-miR812n-3p, osa-miR812n-5p, osa-miR8120-3p, osa-miR8120-5p, osa-miR812p, osa-miR812q, osa-miR812r, osa-miR812s, osa-miR812t, osa-miR812u, osa-miR812v, osa-miR814a, osa-miR814b, osa-miR814c, osa-miR815a, osa-miR815b, osa-miR815c, osa-miR816, osa-miR817, osa-miR818a, osa-miR818b, osa-miR818c, osa-miR818d, osa-miR818e, osa-miR818f, osa-miR820a, osa-miR820b, osa-miR820c, osa-miR821a, osa-miR821b, osa-miR821c, osa-miR827, pab-miR1083, pab-miR11402, pab-miR11403, pab-miR11404, pab-miR11405, pab-miR11406, pab-miR11407a, pab-miR11407b, pab-miR11408a, pab-miR11408b, pab-miR11408c, pab-miR11409a, pab-miR11409b, pab-miR11409c, pab-miR11409d, pab-miR11409e, pab-miR11409f, pab-miR11409g, pab-miR11410, pab-miR11411a, pab-miR11411b, pab-miR11411c, pab-miR11411d, pab-miR11412, pab-miR11413a, pab-miR11413b, pab-miR11414, pab-miR11415, pab-miR11416, pab-miR11417, pab-miR11418, pab-miR11419, pab-miR11420a, pab-miR11420b, pab-miR11420c, pab-miR11420d, pab-miR11420e, pab-miR11420f, pab-miR11420g, pab-miR11421, pab-miR11422a, pab-miR11422b, pab-miR11423a, pab-miR11423b, pab-miR11423c, pab-miR11423d, pab-miR11423e, pab-miR11423f, pab-miR11423g, pab-miR11423h, pab-miR11423i, pab-miR11423j, pab-miR11423k, pab-miR114231, pab-miR11424, pab-miR11425a, pab-miR11425b, pab-miR11425c, pab-miR11425d, pab-miR11425e, pab-miR11425f, pab-miR11426a, pab-miR11426b, pab-miR11427a, pab-miR11427b, pab-miR11428, pab-miR11429a, pab-miR11429b, pab-miR11429c, pab-miR11429d, pab-miR11430, pab-miR11431a, pab-miR11431b, pab-miR11431c, pab-miR11432, pab-miR11433, pab-miR11434, pab-miR11435, pab-miR11436a, pab-miR11436b, pab-miR11436c, pab-miR11436d, pab-miR11437, pab-miR11438, pab-miR11439, pab-miR11440a, pab-miR11440b, pab-miR11441a, pab-miR11441b, pab-miR11442, pab-miR11443, pab-miR11444, pab-miR11445a, pab-miR11445b, pab-miR11445c, pab-miR11446, pab-miR11447, pab-miR11448, pab-miR11449, pab-miR11450, pab-miR11451, pab-miR11452, pab-miR11453, pab-miR11454a, pab-miR11454b, pab-miR11455, pab-miR11456, pab-miR11457, pab-miR11458, pab-miR11459, pab-miR11460, pab-miR11461, pab-miR11462, pab-miR11463, pab-miR11464a, pab-miR11464b, pab-miR11465, pab-miR11466a, pab-miR11466b, pab-miR11466c, pab-miR11466d, pab-miR11466e, pab-miR11467a, pab-miR11467b, pab-miR11468, pab-miR11469, pab-miR11470, pab-miR11471, pab-miR11472, pab-miR11473, pab-miR11474, pab-miR11475, pab-miR11476a, pab-miR11476b, pab-miR11477, pab-miR11478, pab-miR11479, pab-miR11480, pab-miR11481a, pab-miR11481b, pab-miR11481c, pab-miR11482, pab-miR11483, pab-miR11484, pab-miR11485, pab-miR11486, pab-miR11487a, pab-miR11487b, pab-miR11487c, pab-miR11488, pab-miR11489, pab-miR11490a, pab-miR11490b, pab-miR11491, pab-miR11492, pab-miR11493a, pab-miR11493b, pab-miR11493c, pab-miR11494, pab-miR11495, pab-miR11496, pab-miR11497, pab-miR11498a, pab-miR11498b, pab-miR11498c, pab-miR11499a, pab-miR11499b, pab-miR11500, pab-miR11501, pab-miR11502, pab-miR11503, pab-miR11504, pab-miR11505, pab-miR11506, pab-miR11507, pab-miR11508, pab-miR11509, pab-miR11510, pab-miR11511, pab-miR11512, pab-miR11513, pab-miR11514, pab-miR11515a, pab-miR11515b, pab-miR11516, pab-miR11517, pab-miR11518, pab-miR11519, pab-miR11520, pab-miR11521, pab-miR11522, pab-miR11523a, pab-miR11523b, pab-miR11523c, pab-miR11523d, pab-miR11524, pab-miR11525a, pab-miR11525b, pab-miR11526, pab-miR11527, pab-miR11528, pab-miR11529a, pab-miR11529b, pab-miR11529c, pab-miR11530, pab-miR11531, pab-miR11532, pab-miR11533a, pab-miR11533b, pab-miR11534, pab-miR11535, pab-miR11536, pab-miR11537, pab-miR11538, pab-miR11539a, pab-miR11539b, pab-miR11540, pab-miR11541, pab-miR11542, pab-miR11543, pab-miR11544, pab-miR11545, pab-miR11546a, pab-miR11546b, pab-miR11546c, pab-miR11546d, pab-miR11546e, pab-miR11546f, pab-miR11547, pab-miR11548, pab-miR11549, pab-miR11550, pab-miR11551a, pab-miR11551b, pab-miR11552, pab-miR11553, pab-miR11554, pab-miR11555, pab-miR11556, pab-miR11557, pab-miR11558, pab-miR11559, pab-miR11560, pab-miR11561, pab-miR11562a, pab-miR11562b, pab-miR11562c, pab-miR11563, pab-miR11564a, pab-miR11564b, pab-miR11565, pab-miR11566, pab-miR11567, pab-miR11568, pab-miR11569, pab-miR11570a, pab-miR11570b, pab-miR11570c, pab-miR11571, pab-miR11572, pab-miR11573a, pab-miR11573b, pab-miR11574, pab-miR11575, pab-miR11576, pab-miR11577, pab-miR11578, pab-miR11579, pab-miR11580, pab-miR11581, pab-miR11582, pab-miR11583a, pab-miR11583b, pab-miR1311a-3p, pab-miR1311a-5p, pab-miR1311b, pab-miR1312a, pab-miR1312b, pab-miR1312c, pab-miR1313a, pab-miR1313b, pab-miR1313c, pab-miR1313d, pab-miR1314, pab-miR156a, pab-miR156aa, pab-miR156ab, pab-miR156b, pab-miR156c, pab-miR156d, pab-miR156e, pab-miR156f, pab-miR156g, pab-miR156h, pab-miR156i, pab-miR156j, pab-miR156k, pab-miR1561, pab-miR156m, pab-miR156n, pab-miR1560, pab-miR156p, pab-miR156q, pab-miR156r, pab-miR156s, pab-miR156t, pab-miR156u, pab-miR156v, pab-miR156w, pab-miR156x, pab-miR156y, pab-miR156z, pab-miR159a, pab-miR159b, pab-miR159c, pab-miR159d, pab-miR159e, pab-miR159f, pab-miR159g, pab-miR159h, pab-miR159i, pab-miR159j, pab-miR159k, pab-miR160a, pab-miR160b, pab-miR160c, pab-miR160d, pab-miR160e, pab-miR160f, pab-miR162a, pab-miR162b, pab-miR162c, pab-miR164a, pab-miR164b, pab-miR166a, pab-miR166b, pab-miR166c, pab-miR166d, pab-miR166e, pab-miR166f, pab-miR166g, pab-miR166h, pab-miR166i, pab-miR166j, pab-miR167a, pab-miR167b, pab-miR168a, pab-miR168b, pab-miR169a, pab-miR169b, pab-miR169c, pab-miR169d, pab-miR169e, pab-miR171a, pab-miR171b, pab-miR171c, pab-miR171d, pab-miR171e, pab-miR1863, pab-miR2111, pab-miR2950a, pab-miR2950b, pab-miR319a, pab-miR319b, pab-miR319c, pab-miR319d, pab-miR319e, pab-miR319f, pab-miR319g, pab-miR319h, pab-miR319i, pab-miR319j, pab-miR319k, pab-miR3191, pab-miR319m, pab-miR319n, pab-miR3627a, pab-miR3627b, pab-miR3627c, pab-miR3627d, pab-miR3627e, pab-miR3627f, pab-miR3627g, pab-miR3627h, pab-miR3627i, pab-miR3627j, pab-miR3693, pab-miR3693b, pab-miR3693c, pab-miR3693d, pab-miR3693e, pab-miR3693f, pab-miR3693g, pab-miR3693h, pab-miR3693i, pab-miR3693j, pab-miR3693k, pab-miR3694, pab-miR3695a, pab-miR3695b, pab-miR3695c, pab-miR3695d-3p, pab-miR3695d-5p, pab-miR3696, pab-miR3697a, pab-miR3697b, pab-miR3697c, pab-miR3698, pab-miR3698b, pab-miR3699, pab-miR3700, pab-miR3701a, pab-miR3701b-3p, pab-miR3701b-5p, pab-miR3701c, pab-miR3701d, pab-miR3701e, pab-miR3701f, pab-miR3701g, pab-miR3701h, pab-miR3701i, pab-miR3701j, pab-miR3702, pab-miR3703, pab-miR3704, pab-miR3705, pab-miR3706, pab-miR3707, pab-miR3708, pab-miR3709a, pab-miR3709b, pab-miR3710, pab-miR3710b, pab-miR3710c, pab-miR3710d, pab-miR3710e, pab-miR3710f, pab-miR3710g, pab-miR3710h, pab-miR3710i, pab-miR3711, pab-miR3712, pab-miR390a, pab-miR390b, pab-miR391a, pab-miR391b, pab-miR391c, pab-miR391d, pab-miR391e, pab-miR391f, pab-miR391g, pab-miR391h, pab-miR391i, pab-miR393a, pab-miR393b, pab-miR393c, pab-miR393d, pab-miR394a, pab-miR394b, pab-miR394c, pab-miR395, pab-miR395a, pab-miR395b, pab-miR395c, pab-miR395d, pab-miR395e, pab-miR395f, pab-miR395g, pab-miR395h, pab-miR395i, pab-miR396a-3p, pab-miR396a-5p, pab-miR396b, pab-miR396c, pab-miR396d, pab-miR396e, pab-miR396f, pab-miR396g, pab-miR396h, pab-miR396i, pab-miR396j, pab-miR396k, pab-miR3961, pab-miR396m, pab-miR396n, pab-miR3960, pab-miR396p, pab-miR396q, pab-miR396r, pab-miR396s, pab-miR396t, pab-miR396u, pab-miR397a, pab-miR397b, pab-miR397c, pab-miR397d, pab-miR397e, pab-miR397f, pab-miR397g, pab-miR397h, pab-miR397i, pab-miR397j, pab-miR397k, pab-miR3971, pab-miR397m, pab-miR397n, pab-miR397o, pab-miR397p, pab-miR397q, pab-miR397r, pab-miR397s, pab-miR397t, pab-miR397u, pab-miR398a, pab-miR398b, pab-miR399a, pab-miR399b, pab-miR399c, pab-miR399d, pab-miR399e, pab-miR399f, pab-miR399g, pab-miR399h, pab-miR408, pab-miR4414, pab-miR472, pab-miR482a, pab-miR482b, pab-miR482c, pab-miR482d-3p, pab-miR482d-5p, pab-miR482e, pab-miR482f, pab-miR482g, pab-miR482h, pab-miR482i, pab-miR482j, pab-miR482k, pab-miR4821, pab-miR482m, pab-miR482n, pab-miR4820, pab-miR482p, pab-miR482q, pab-miR482r, pab-miR482s, pab-miR482t, pab-miR482u, pab-miR482v, pab-miR482w, pab-miR482x, pab-miR529a, pab-miR529b, pab-miR529c, pab-miR529d, pab-miR529e, pab-miR529f, pab-miR529g, pab-miR529h, pab-miR529i, pab-miR529j, pab-miR529k, pab-miR5291, pab-miR529m, pab-miR529n, pab-miR5290, pab-miR529p, pab-miR535a, pab-miR535b, pab-miR535c, pab-miR535d, pab-miR535e, pab-miR536a, pab-miR536b, pab-miR828, pab-miR858a, pab-miR858b, pab-miR946a, pab-miR946b, pab-miR946c, pab-miR946d, pab-miR946e, pab-miR946f, pab-miR946g, pab-miR946h, pab-miR947, pab-miR949a, pab-miR949b, pab-miR949c, pab-miR950a-3p, pab-miR950a-5p, pab-miR950b, pab-miR950c, pab-miR950d, pab-miR950e, pab-miR950f, pab-miR950g, pab-miR950h, pab-miR950i, pab-miR951, pal-let-7a-2-3p, pal-let-7a-3p, pal-let-7a-5p, pal-let-7c-3p, pal-let-7c-5p, pal-let-7e-3p, pal-let-7e-5p, pal-let-7f-3p, pal-let-7f-5p, pal-let-7g-3p, pal-let-7g-5p, pal-let-7i-3p, pal-let-7i-5p, pal-miR-1-2-5p, pal-miR-1-3p, pal-miR-1-5p, pal-miR-100-3p, pal-miR-100-5p, pal-miR-10000-3p, pal-miR-10000-5p, pal-miR-10001-3p, pal-miR-10001-5p, pal-miR-10002-3p, pal-miR-10002-5p, pal-miR-10003-3p, pal-miR-10003-5p, pal-miR-10004-3p, pal-miR-10004-5p, pal-miR-10005-3p, pal-miR-10005-5p, pal-miR-10006-3p, pal-miR-10006-5p, pal-miR-10007-3p, pal-miR-10007-5p, pal-miR-10008-3p, pal-miR-10008-5p, pal-miR-10009-3p, pal-miR-10009-5p, pal-miR-10010-3p, pal-miR-10010-5p, pal-miR-10011-3p, pal-miR-10011-5p, pal-miR-10012-3p, pal-miR-10012-5p, pal-miR-10013-3p, pal-miR-10013-5p, pal-miR-10014-3p, pal-miR-10014-5p, pal-miR-10015-3p, pal-miR-10015-5p, pal-miR-10016-3p, pal-miR-10016-5p, pal-miR-10017-2-5p, pal-miR-10017-3p, pal-miR-10017-5p, pal-miR-10018-3p, pal-miR-10018-5p, pal-miR-10019-3p, pal-miR-10019-5p, pal-miR-101-2-5p, pal-miR-101-3p, pal-miR-101-5p, pal-miR-103a-3p, pal-miR-103a-5p, pal-miR-103b-3p, pal-miR-103b-5p, pal-miR-105-3p, pal-miR-105-5p, pal-miR-106a-3p, pal-miR-106a-5p, pal-miR-106b-3p, pal-miR-106b-5p, pal-miR-107a-3p, pal-miR-107a-5p, pal-miR-107b-3p, pal-miR-107b-5p, pal-miR-10a-3p, pal-miR-10a-5p, pal-miR-10b-3p, pal-miR-10b-5p, pal-miR-1180-3p, pal-miR-1180-5p, pal-miR-1185-3p, pal-miR-1185-5p, pal-miR-122-3p, pal-miR-122-5p, pal-miR-1224-3p, pal-miR-1224-5p, pal-miR-124-3p, pal-miR-124-5p, pal-miR-1249-3p, pal-miR-1249-5p, pal-miR-125a-3p, pal-miR-125a-5p, pal-miR-125b-2-3p, pal-miR-125b-3p, pal-miR-125b-5p, pal-miR-126-3p, pal-miR-126-5p, pal-miR-127-3p, pal-miR-127-5p, pal-miR-128-3p, pal-miR-128-5p, pal-miR-1296-3p, pal-miR-1296-5p, pal-miR-129a-3p, pal-miR-129a-5p, pal-miR-129b-3p, pal-miR-129b-5p, pal-miR-130-3p, pal-miR-130-5p, pal-miR-1301-3p, pal-miR-1301-5p, pal-miR-1306-3p, pal-miR-1306-5p, pal-miR-1307-3p, pal-miR-1307-5p, pal-miR-132-3p, pal-miR-132-5p, pal-miR-133a-3p, pal-miR-133a-5p, pal-miR-133b-3p, pal-miR-133b-5p, pal-miR-135-2-3p, pal-miR-135-3p, pal-miR-135-5p, pal-miR-136-3p, pal-miR-136-5p, pal-miR-137-3p, pal-miR-137-5p, pal-miR-1379-3p, pal-miR-1379-5p, pal-miR-138-2-3p, pal-miR-138-3p, pal-miR-138-5p, pal-miR-1388-3p, pal-miR-1388-5p, pal-miR-139-3p, pal-miR-139-5p, pal-miR-140-3p, pal-miR-140-5p, pal-miR-141-3p, pal-miR-141-5p, pal-miR-142-3p, pal-miR-142-5p, pal-miR-143-3p, pal-miR-143-5p, pal-miR-144-3p, pal-miR-144-5p, pal-miR-145-3p, pal-miR-145-5p, pal-miR-146a-3p, pal-miR-146a-5p, pal-miR-146b-3p, pal-miR-146b-5p, pal-miR-148a-3p, pal-miR-148a-5p, pal-miR-148b-3p, pal-miR-148b-5p, pal-miR-150-3p, pal-miR-150-5p, pal-miR-151-3p, pal-miR-151-5p, pal-miR-152-3p, pal-miR-152-5p, pal-miR-153-2-5p, pal-miR-153-3p, pal-miR-153-5p, pal-miR-154b-3p, pal-miR-154b-5p, pal-miR-155-3p, pal-miR-155-5p, pal-miR-15a-3p, pal-miR-15a-5p, pal-miR-15b-3p, pal-miR-15b-5p, pal-miR-16-2-3p, pal-miR-16-3p, pal-miR-16-5p, pal-miR-17-3p, pal-miR-17-5p, pal-miR-181a-2-3p, pal-miR-181a-3p, pal-miR-181a-5p, pal-miR-181b-2-3p, pal-miR-181b-3p, pal-miR-181b-5p, pal-miR-181c-3p, pal-miR-181c-5p, pal-miR-181d-3p, pal-miR-181d-5p, pal-miR-182-3p, pal-miR-182-5p, pal-miR-183-3p, pal-miR-183-5p, pal-miR-1839-3p, pal-miR-1839-5p, pal-miR-184-3p, pal-miR-184-5p, pal-miR-185-3p, pal-miR-185-5p, pal-miR-186-3p, pal-miR-186-5p, pal-miR-187-3p, pal-miR-187-5p, pal-miR-188-3p, pal-miR-188-5p, pal-miR-18a-3p, pal-miR-18a-5p, pal-miR-18b-3p, pal-miR-18b-5p, pal-miR-19-2-5p, pal-miR-19-3p, pal-miR-19-5p, pal-miR-190b-3p, pal-miR-190b-5p, pal-miR-191-3p, pal-miR-191-5p, pal-miR-1911-3p, pal-miR-1911-5p, pal-miR-192-3p, pal-miR-192-5p, pal-miR-193a-3p, pal-miR-193a-5p, pal-miR-193b-3p, pal-miR-193b-5p, pal-miR-194a-3p, pal-miR-194a-5p, pal-miR-194b-3p, pal-miR-194b-5p, pal-miR-195-3p, pal-miR-195-5p, pal-miR-196a-3p, pal-miR-196a-5p, pal-miR-196b-3p, pal-miR-196b-5p, pal-miR-196c-3p, pal-miR-196c-5p, pal-miR-199-3-5p, pal-miR-199-3p, pal-miR-199-5p, pal-miR-200a-3p, pal-miR-200a-5p, pal-miR-200b-3p, pal-miR-200b-5p, pal-miR-200c-3p, pal-miR-200c-5p, pal-miR-202-3p, pal-miR-202-5p, pal-miR-204-3p, pal-miR-204-5p, pal-miR-205-3p, pal-miR-205-5p, pal-miR-206-3p, pal-miR-206-5p, pal-miR-208a-3p, pal-miR-208a-5p, pal-miR-208b-3p, pal-miR-208b-5p, pal-miR-20a-3p, pal-miR-20a-5p, pal-miR-20b-3p, pal-miR-20b-5p, pal-miR-21-3p, pal-miR-21-5p, pal-miR-211-3p, pal-miR-211-5p, pal-miR-212-3p, pal-miR-212-5p, pal-miR-214-3p, pal-miR-214-5p, pal-miR-215-3p, pal-miR-215-5p, pal-miR-216b-3p, pal-miR-216b-5p, pal-miR-218-2-3p, pal-miR-218-3p, pal-miR-218-5p, pal-miR-219a-2-3p, pal-miR-219a-3p, pal-miR-219a-5p, pal-miR-219b-3p, pal-miR-219b-5p, pal-miR-22-3p, pal-miR-22-5p, pal-miR-221-3p, pal-miR-221-5p, pal-miR-222-3p, pal-miR-222-5p, pal-miR-224-3p, pal-miR-224-5p, pal-miR-23a-3p, pal-miR-23a-5p, pal-miR-23b-3p, pal-miR-23b-5p, pal-miR-25-3p, pal-miR-25-5p, pal-miR-26a-2-3p, pal-miR-26a-3p, pal-miR-26a-5p, pal-miR-26b-3p, pal-miR-26b-5p, pal-miR-27a-3p, pal-miR-27a-5p, pal-miR-27b-3p, pal-miR-27b-5p, pal-miR-28-3p, pal-miR-28-5p, pal-miR-282-3p, pal-miR-282-5p, pal-miR-296-3p, pal-miR-296-5p, pal-miR-299a-3p, pal-miR-299a-5p, pal-miR-299b-3p, pal-miR-299b-5p, pal-miR-29a-3p, pal-miR-29a-5p, pal-miR-29b-2-5p, pal-miR-29b-3p, pal-miR-29b-5p, pal-miR-29c-3p, pal-miR-29c-5p, pal-miR-301a-3p, pal-miR-301a-5p, pal-miR-301b-3p, pal-miR-301b-5p, pal-miR-3059-3p, pal-miR-3059-5p, pal-miR-3064-3p, pal-miR-3064-5p, pal-miR-3085-3p, pal-miR-3085-5p, pal-miR-30a-3p, pal-miR-30a-5p, pal-miR-30b-3p, pal-miR-30b-5p, pal-miR-30c-3p, pal-miR-30c-5p, pal-miR-30d-3p, pal-miR-30d-5p, pal-miR-30e-3p, pal-miR-30e-5p, pal-miR-31-3p, pal-miR-31-5p, pal-miR-3120-3p, pal-miR-3120-5p, pal-miR-32-3p, pal-miR-32-5p, pal-miR-320-3p, pal-miR-320-5p, pal-miR-323-3p, pal-miR-323-5p, pal-miR-323b-3p, pal-miR-323b-5p, pal-miR-324-3p, pal-miR-324-5p, pal-miR-329b-3p, pal-miR-329b-5p, pal-miR-330-3p, pal-miR-330-5p, pal-miR-331-3p, pal-miR-331-5p, pal-miR-335-3p, pal-miR-335-5p, pal-miR-337-3p, pal-miR-337-5p, pal-miR-338-3p, pal-miR-338-5p, pal-miR-339a-3p, pal-miR-339a-5p, pal-miR-339b-3p, pal-miR-339b-5p, pal-miR-33a-3p, pal-miR-33a-5p, pal-miR-33b-3p, pal-miR-33b-5p, pal-miR-340-3p, pal-miR-340-5p, pal-miR-342-3p, pal-miR-342-5p, pal-miR-345-3p, pal-miR-345-5p, pal-miR-346-3p, pal-miR-346-5p, pal-miR-34a-3p, pal-miR-34a-5p, pal-miR-34b-3p, pal-miR-34b-5p, pal-miR-34c-3p, pal-miR-34c-5p, pal-miR-361-3p, pal-miR-361-5p, pal-miR-362-3p, pal-miR-362-5p, pal-miR-363-3p, pal-miR-363-5p, pal-miR-365b-3p, pal-miR-365b-5p, pal-miR-369-3p, pal-miR-369-5p, pal-miR-370-3p, pal-miR-370-5p, pal-miR-371-3p, pal-miR-371-5p, pal-miR-374b-3p, pal-miR-374b-5p, pal-miR-375-3p, pal-miR-375-5p, pal-miR-376a-3p, pal-miR-376a-5p, pal-miR-376b-3p, pal-miR-376b-5p, pal-miR-376c-3p, pal-miR-376c-5p, pal-miR-376d-3p, pal-miR-376d-5p, pal-miR-377-3p, pal-miR-377-5p, pal-miR-378-3p, pal-miR-378-5p, pal-miR-378c-3p, pal-miR-378c-5p, pal-miR-378d-3p, pal-miR-378d-5p, pal-miR-378e-3p, pal-miR-378e-5p, pal-miR-379-3p, pal-miR-379-5p, pal-miR-380-3p, pal-miR-380-5p, pal-miR-381-3p, pal-miR-381-5p, pal-miR-382-3p, pal-miR-382-5p, pal-miR-383-3p, pal-miR-383-5p, pal-miR-3958-3p, pal-miR-3958-5p, pal-miR-3959-3p, pal-miR-3959-5p, pal-miR-409-3p, pal-miR-409-5p, pal-miR-410-3p, pal-miR-410-5p, pal-miR-411-3p, pal-miR-411-5p, pal-miR-412-3p, pal-miR-412-5p, pal-miR-421-3p, pal-miR-421-5p, pal-miR-422c-3p, pal-miR-422c-5p, pal-miR-423-3p, pal-miR-423-5p, pal-miR-424-3p, pal-miR-424-5p, pal-miR-425-3p, pal-miR-425-5p, pal-miR-429-3p, pal-miR-429-5p, pal-miR-432-3p, pal-miR-432-5p, pal-miR-433-3p, pal-miR-433-5p, pal-miR-449a-3p, pal-miR-449a-5p, pal-miR-449c-3p, pal-miR-449c-5p, pal-miR-450a-2-3p, pal-miR-450a-3p, pal-miR-450a-5p, pal-miR-450b-3p, pal-miR-450b-5p, pal-miR-452-3p, pal-miR-452-5p, pal-miR-454-3p, pal-miR-454-5p, pal-miR-458-3p, pal-miR-458-5p, pal-miR-4646-3p, pal-miR-4646-5p, pal-miR-4657-3p, pal-miR-4657-5p, pal-miR-484-3p, pal-miR-484-5p, pal-miR-485-3p, pal-miR-485-5p, pal-miR-486a-3-3p, pal-miR-486a-3p, pal-miR-486a-5p, pal-miR-487a-3p, pal-miR-487a-5p, pal-miR-487b-3p, pal-miR-487b-5p, pal-miR-490-3p, pal-miR-490-5p, pal-miR-493-3p, pal-miR-493-5p, pal-miR-495-3p, pal-miR-495-5p, pal-miR-497-3p, pal-miR-497-5p, pal-miR-499a-3p, pal-miR-499a-5p, pal-miR-500-3p, pal-miR-500-5p, pal-miR-502-3p, pal-miR-502-5p, pal-miR-503-3p, pal-miR-503-5p, pal-miR-504-3p, pal-miR-504-5p, pal-miR-506-3p, pal-miR-506-5p, pal-miR-507-3p, pal-miR-507-5p, pal-miR-509b-3p, pal-miR-509b-5p, pal-miR-513-3p, pal-miR-513-5p, pal-miR-532-3p, pal-miR-532-5p, pal-miR-541-3p, pal-miR-541-5p, pal-miR-542-3p, pal-miR-542-5p, pal-miR-543-3p, pal-miR-543-5p, pal-miR-544-3p, pal-miR-544-5p, pal-miR-551b-3p, pal-miR-551b-5p, pal-miR-574-3p, pal-miR-574-5p, pal-miR-599-3p, pal-miR-599-5p, pal-miR-6119-3p, pal-miR-6119-5p, pal-miR-628-3p, pal-miR-628-5p, pal-miR-652-3p, pal-miR-652-5p, pal-miR-6529-3p, pal-miR-6529-5p, pal-miR-656-3p, pal-miR-656-5p, pal-miR-660-3p, pal-miR-660-5p, pal-miR-664a-2-5p, pal-miR-664a-3p, pal-miR-664a-5p, pal-miR-664b-3p, pal-miR-664b-5p, pal-miR-665-3p, pal-miR-665-5p, pal-miR-668-3p, pal-miR-668-5p, pal-miR-672-3p, pal-miR-672-5p, pal-miR-708-3p, pal-miR-708-5p, pal-miR-7140-3p, pal-miR-7140-5p, pal-miR-758-3p, pal-miR-758-5p, pal-miR-760-3p, pal-miR-760-5p, pal-miR-7a-3p, pal-miR-7a-5p, pal-miR-7b-2-3p, pal-miR-7b-3p, pal-miR-7b-5p, pal-miR-802-3p, pal-miR-802-5p, pal-miR-873-3p, pal-miR-873-5p, pal-miR-874-3p, pal-miR-874-5p, pal-miR-876-3p, pal-miR-876-5p, pal-miR-885-3p, pal-miR-885-5p, pal-miR-889-3p, pal-miR-889-5p, pal-miR-8908a-2-3p, pal-miR-8908a-3p, pal-miR-8908a-5p, pal-miR-8908b-3p, pal-miR-8908b-5p, pal-miR-8908c-3p, pal-miR-8908c-5p, pal-miR-8908d-3p, pal-miR-8908d-5p, pal-miR-8908e-3p, pal-miR-8908e-5p, pal-miR-8908f-3p, pal-miR-8908f-5p, pal-miR-8908g-3p, pal-miR-8908g-5p, pal-miR-9-3p, pal-miR-9-5p, pal-miR-9226-3p, pal-miR-9226-5p, pal-miR-9287-3p, pal-miR-9287-5p, pal-miR-9298-3p, pal-miR-9298-5p, pal-miR-92a-2-5p, pal-miR-92a-3p, pal-miR-92a-5p, pal-miR-92b-3p, pal-miR-92b-5p, pal-miR-93-3p, pal-miR-93-5p, pal-miR-96-3p, pal-miR-96-5p, pal-miR-98-3p, pal-miR-98-5p, pal-miR-9987-3p, pal-miR-9987-5p, pal-miR-9988-3p, pal-miR-9988-5p, pal-miR-9989-3p, pal-miR-9989-5p, pal-miR-9990-3p, pal-miR-9990-5p, pal-miR-9991-3p, pal-miR-9991-5p, pal-miR-9992-3p, pal-miR-9992-5p, pal-miR-9993a-3p, pal-miR-9993a-5p, pal-miR-9993b-3p, pal-miR-9993b-5p, pal-miR-9994-3p, pal-miR-9994-5p, pal-miR-9995-3p, pal-miR-9995-5p, pal-miR-9996-3p, pal-miR-9996-5p, pal-miR-9997-3p, pal-miR-9997-5p, pal-miR-9998-3p, pal-miR-9998-5p, pal-miR-9999-3p, pal-miR-9999-5p, pal-miR-99a-3p, pal-miR-99a-5p, pal-miR-99b-3p, pal-miR-99b-5p, pbi-miR-506, pbi-miR-507, pbi-miR-508, pbi-miR-509, pbi-miR-510, pbi-miR-513a, pbi-miR-513b, pbi-miR-513c, pbi-miR-514, pbv-let-7a-2-3p, pbv-let-7a-3p, pbv-let-7a-5p, pbv-let-7b-3p, pbv-let-7b-5p, pbv-let-7c-2-3p, pbv-let-7c-3p, pbv-let-7c-5p, pbv-let-7d-3p, pbv-let-7d-5p, pbv-let-7e-3p, pbv-let-7e-5p, pbv-let-7f-2-3p, pbv-let-7f-3p, pbv-let-7f-5p, pbv-let-7g-3p, pbv-let-7g-5p, pbv-let-7i-3p, pbv-let-7i-5p, pbv-miR-100-3p, pbv-miR-100-5p, pbv-miR-101-2-5p, pbv-miR-101-3p, pbv-miR-101-5p, pbv-miR-103-3p, pbv-miR-106-3p, pbv-miR-106-5p, pbv-miR-107-3p, pbv-miR-107-5p, pbv-miR-10a-3p, pbv-miR-10a-5p, pbv-miR-10b-2-3p, pbv-miR-10b-3p, pbv-miR-10b-5p, pbv-miR-122-3p, pbv-miR-122-5p, pbv-miR-124a-3p, pbv-miR-124a-5p, pbv-miR-124b-3p, pbv-miR-124b-5p, pbv-miR-125a-5p, pbv-miR-125b-3p, pbv-miR-125b-5p, pbv-miR-126-3p, pbv-miR-126-5p, pbv-miR-128-3p, pbv-miR-128-5p, pbv-miR-129a-3p, pbv-miR-129a-5p, pbv-miR-129b-3p, pbv-miR-129b-5p, pbv-miR-1306-3p, pbv-miR-130a-3p, pbv-miR-130a-5p, pbv-miR-130b-3p, pbv-miR-130c-3p, pbv-miR-130c-5p, pbv-miR-130d-3p, pbv-miR-130d-5p, pbv-miR-132-3p, pbv-miR-132-5p, pbv-miR-1329-3p, pbv-miR-1329-5p, pbv-miR-133a-2-5p, pbv-miR-133a-3p, pbv-miR-133a-5p, pbv-miR-133b-3p, pbv-miR-133b-5p, pbv-miR-133c-3p, pbv-miR-135-2-3p, pbv-miR-135-3-3p, pbv-miR-135-3p, pbv-miR-135-5p, pbv-miR-137a-3p, pbv-miR-137a-5p, pbv-miR-137b-3p, pbv-miR-137b-5p, pbv-miR-138-3p, pbv-miR-138-5p, pbv-miR-1388-3p, pbv-miR-1388-5p, pbv-miR-139-3p, pbv-miR-139-5p, pbv-miR-1397-3p, pbv-miR-1397-5p, pbv-miR-142-3p, pbv-miR-142-5p, pbv-miR-143-3p, pbv-miR-143-5p, pbv-miR-144-3p, pbv-miR-144-5p, pbv-miR-145-3p, pbv-miR-145-5p, pbv-miR-146a-5p, pbv-miR-146b-5p, pbv-miR-147-3p, pbv-miR-148a-3p, pbv-miR-148a-5p, pbv-miR-148b-3p, pbv-miR-148b-5p, pbv-miR-150-3p, pbv-miR-150-5p, pbv-miR-153-2-5p, pbv-miR-153-3p, pbv-miR-153-5p, pbv-miR-155a-5p, pbv-miR-155b-5p, pbv-miR-15a-3p, pbv-miR-15a-5p, pbv-miR-15b-3p, pbv-miR-15b-5p, pbv-miR-1641-5p, pbv-miR-1662-5p, pbv-miR-1677-3p, pbv-miR-1677-5p, pbv-miR-16a-5p, pbv-miR-16b-3p, pbv-miR-16b-5p, pbv-miR-16c-3p, pbv-miR-16c-5p, pbv-miR-17-3p, pbv-miR-17-5p, pbv-miR-1788-3p, pbv-miR-1788-5p, pbv-miR-1805-3p, pbv-miR-1805-5p, pbv-miR-181a-3p, pbv-miR-181a-5p, pbv-miR-181b-3p, pbv-miR-181b-5p, pbv-miR-181c-5p, pbv-miR-182-3p, pbv-miR-182-5p, pbv-miR-183-5p, pbv-miR-184-3p, pbv-miR-18a-3p, pbv-miR-18a-5p, pbv-miR-18b-5p, pbv-miR-190a-3p, pbv-miR-190a-5p, pbv-miR-190b-5p, pbv-miR-191-3p, pbv-miR-191-5p, pbv-miR-193a-3p, pbv-miR-193a-5p, pbv-miR-193b-3p, pbv-miR-194-5p, pbv-miR-196a-3p, pbv-miR-196a-5p, pbv-miR-196c-5p, pbv-miR-199-3-5p, pbv-miR-199-3p, pbv-miR-199-5p, pbv-miR-19a-3p, pbv-miR-19a-5p, pbv-miR-19b-3p, pbv-miR-1a-2-5p, pbv-miR-1a-3p, pbv-miR-1a-5p, pbv-miR-1b-3p, pbv-miR-1b-5p, pbv-miR-200a-3p, pbv-miR-200a-5p, pbv-miR-200b-3p, pbv-miR-200b-5p, pbv-miR-202-5p, pbv-miR-203-3p, pbv-miR-203-5p, pbv-miR-204-3-3p, pbv-miR-204-3p, pbv-miR-204-5p, pbv-miR-205a-5p, pbv-miR-205b-5p, pbv-miR-206-3p, pbv-miR-208-3p, pbv-miR-208-5p, pbv-miR-20a-5p, pbv-miR-20b-3p, pbv-miR-20b-5p, pbv-miR-21-3p, pbv-miR-21-5p, pbv-miR-210-3p, pbv-miR-210-5p, pbv-miR-212-3p, pbv-miR-212-5p, pbv-miR-214-3p, pbv-miR-214-5p, pbv-miR-215-3p, pbv-miR-215-5p, pbv-miR-216a-3p, pbv-miR-216a-5p, pbv-miR-216b-3p, pbv-miR-216b-5p, pbv-miR-217-5p, pbv-miR-218-3p, pbv-miR-218-5p, pbv-miR-2188-3p, pbv-miR-2188-5p, pbv-miR-219-3p, pbv-miR-219-5p, pbv-miR-22-3p, pbv-miR-22-5p, pbv-miR-221-3p, pbv-miR-221-5p, pbv-miR-222a-3p, pbv-miR-222a-5p, pbv-miR-222b-3p, pbv-miR-222b-5p, pbv-miR-223-3p, pbv-miR-23a-3p, pbv-miR-23b-3p, pbv-miR-23b-5p, pbv-miR-24-3p, pbv-miR-24-5p, pbv-miR-26-3-3p, pbv-miR-26-3p, pbv-miR-26-5p, pbv-miR-27a-3p, pbv-miR-27a-5p, pbv-miR-27b-3p, pbv-miR-27b-5p, pbv-miR-2970-3p, pbv-miR-2970-5p, pbv-miR-29a-2-5p, pbv-miR-29a-3p, pbv-miR-29a-5p, pbv-miR-29b-3p, pbv-miR-29b-5p, pbv-miR-301a-3p, pbv-miR-301a-5p, pbv-miR-301b-3p, pbv-miR-3064-3p, pbv-miR-30a-3p, pbv-miR-30a-5p, pbv-miR-30b-5p, pbv-miR-30c-2-3p, pbv-miR-30c-3p, pbv-miR-30c-5p, pbv-miR-30d-3p, pbv-miR-30d-5p, pbv-miR-30e-3p, pbv-miR-30e-5p, pbv-miR-31-5p, pbv-miR-32-5p, pbv-miR-33-3p, pbv-miR-33-5p, pbv-miR-338-3p, pbv-miR-338-5p, pbv-miR-34a-5p, pbv-miR-34b-3p, pbv-miR-34b-5p, pbv-miR-34c-3p, pbv-miR-34c-5p, pbv-miR-3618-5p, pbv-miR-363-3p, pbv-miR-365-3p, pbv-miR-375-3p, pbv-miR-383-3p, pbv-miR-383-5p, pbv-miR-425-3p, pbv-miR-425-5p, pbv-miR-429-3p, pbv-miR-449a-5p, pbv-miR-449b-3p, pbv-miR-449b-5p, pbv-miR-449c-5p, pbv-miR-449d-5p, pbv-miR-454-3p, pbv-miR-455-3p, pbv-miR-455-5p, pbv-miR-456-3p, pbv-miR-456-5p, pbv-miR-458-3p, pbv-miR-489-3p, pbv-miR-490-3p, pbv-miR-490-5p, pbv-miR-499-3p, pbv-miR-499-5p, pbv-miR-5420-3p, pbv-miR-551a-3p, pbv-miR-551b-3p, pbv-miR-551b-5p, pbv-miR-599-3p, pbv-miR-7-2-3p, pbv-miR-7-3-3p, pbv-miR-7-3p, pbv-miR-7-5p, pbv-miR-737-3p, pbv-miR-737-5p, pbv-miR-875-5p, pbv-miR-9-3p, pbv-miR-9-4-3p, pbv-miR-9-5p, pbv-miR-92a-3p, pbv-miR-92b-3p, pbv-miR-96-3p, pbv-miR-96-5p, pbv-miR-9650-3p, pbv-miR-9650-5p, pbv-miR-9651-3p, pbv-miR-9651-5p, pbv-miR-98-3p, pbv-miR-98-5p, pbv-miR-99a-3p, pbv-miR-99a-5p, pbv-miR-99b-3p, pbv-miR-99b-5p, pca-bantam-3p, pca-bantam-5p, pca-iab-4-3p, pca-iab-4-5p, pca-let-7-3p, pca-let-7-5p, pca-miR-1-3p, pca-miR-1-5p, pca-miR-100-3p, pca-miR-100-5p, pca-miR-1000-3p, pca-miR-1000-5p, pca-miR-11-3p, pca-miR-11-5p, pca-miR-11663-3p, pca-miR-11663-5p, pca-miR-11664a-3p, pca-miR-11664a-5p, pca-miR-11664b-3p, pca-miR-11664b-5p, pca-miR-11665-3p, pca-miR-11665-5p, pca-miR-1175-3p, pca-miR-1175-5p, pca-miR-12-3p, pca-miR-12-5p, pca-miR-124-3p, pca-miR-124-5p, pca-miR-133-3p, pca-miR-133-5p, pca-miR-137-3p, pca-miR-137-5p, pca-miR-13a-3p, pca-miR-13a-5p, pca-miR-13b-3p, pca-miR-13b-5p, pca-miR-14-3p, pca-miR-14-5p, pca-miR-184-3p, pca-miR-184-5p, pca-miR-190-3p, pca-miR-190-5p, pca-miR-210-3p, pca-miR-210-5p, pca-miR-219-3p, pca-miR-219-5p, pca-miR-252-3p, pca-miR-252-5p, pca-miR-263b-3p, pca-miR-263b-5p, pca-miR-275-3p, pca-miR-275-5p, pca-miR-276-3p, pca-miR-276-5p, pca-miR-2765-3p, pca-miR-2765-5p, pca-miR-277-3p, pca-miR-277-5p, pca-miR-278-3p, pca-miR-278-5p, pca-miR-2796-3p, pca-miR-2796-5p, pca-miR-279b-3p, pca-miR-279b-5p, pca-miR-279c-3p, pca-miR-279c-5p, pca-miR-281-3p, pca-miR-281-5p, pca-miR-282-3p, pca-miR-282-5p, pca-miR-283-3p, pca-miR-283-5p, pca-miR-29-3p, pca-miR-3049-3p, pca-miR-3049-5p, pca-miR-305-3p, pca-miR-305-5p, pca-miR-306-3p, pca-miR-306-5p, pca-miR-31-3p, pca-miR-31-5p, pca-miR-315-3p, pca-miR-315-5p, pca-miR-316-3p, pca-miR-316-5p, pca-miR-317-3p, pca-miR-317-5p, pca-miR-33-3p, pca-miR-33-5p, pca-miR-34-3p, pca-miR-34-5p, pca-miR-3736-5p, pca-miR-3759-3p, pca-miR-3770-5p, pca-miR-3782-5p, pca-miR-3783-3p, pca-miR-3783-5p, pca-miR-3785-3p, pca-miR-3785-5p, pca-miR-3786-3p, pca-miR-3786-5p, pca-miR-6001-3p, pca-miR-6001-5p, pca-miR-6038-3p, pca-miR-6038-5p, pca-miR-6039-3p, pca-miR-6039-5p, pca-miR-6065-3p, pca-miR-6067-3p, pca-miR-6067-5p, pca-miR-7-3p, pca-miR-7-5p, pca-miR-71-3p, pca-miR-71-5p, pca-miR-750-3p, pca-miR-750-5p, pca-miR-8-3p, pca-miR-8-5p, pca-miR-9-3p, pca-miR-9-5p, pca-miR-927a-3p, pca-miR-927a-5p, pca-miR-927b-3p, pca-miR-927b-5p, pca-miR-929-3p, pca-miR-929-5p, pca-miR-92c-3p, pca-miR-92c-5p, pca-miR-932-3p, pca-miR-932-5p, pca-miR-965-3p, pca-miR-965-5p, pca-miR-971-3p, pca-miR-971-5p, pca-miR-980-3p, pca-miR-980-5p, pca-miR-981-3p, pca-miR-981-5p, pca-miR-995-3p, pca-miR-995-5p, pde-miR1311, pde-miR1312, pde-miR1313, pde-miR1314, pde-miR159, pde-miR162, pde-miR166a, pde-miR166b, pde-miR169, pde-miR171, pde-miR3701, pde-miR3704a, pde-miR3704b, pde-miR3712, pde-miR390, pde-miR396, pde-miR482a, pde-miR482b, pde-miR482c, pde-miR482d, pde-miR946, pde-miR947, pde-miR949a, pde-miR949b, pde-miR950, pde-miR951, pde-miR952a, pde-miR952b, pde-miR952c, peu-miR2912a, peu-miR2912b, peu-miR2913, peu-miR2916, pgi-miR2118, pgi-miR391, pgi-miR482a, pgi-miR482b, pgi-miR6135a, pgi-miR6135b, pgi-miR6135c, pgi-miR6135d, pgi-miR6135e-5p, pgi-miR6135e.2-5p, pgi-miR6135f, pgi-miR6135g, pgi-miR6135h, pgi-miR6135i, pgi-miR6135j, pgi-miR6135k, pgi-miR6136a.1, pgi-miR6136a.2, pgi-miR6136b, pgi-miR6137a, pgi-miR6137b, pgi-miR6138, pgi-miR6139, pgi-miR6140a, pgi-miR6140b, pgi-miR6140c, pgi-miR6140d, pgi-miR6141, pgi-miR6142, pgi-miR6143a, pgi-miR6143b-3p, pgi-miR6143b-5p, pha-let-7a, pha-let-7b, pha-let-7c, pha-let-7e, pha-let-7f, pha-let-7g, pha-let-7i, pha-miR-100, pha-miR-101, pha-miR-103a, pha-miR-103b, pha-miR-106b, pha-miR-107, pha-miR-10a, pha-miR-10b, pha-miR-125a, pha-miR-125b, pha-miR-127, pha-miR-1307, pha-miR-130a, pha-miR-130b, pha-miR-132, pha-miR-140, pha-miR-143, pha-miR-145, pha-miR-146a, pha-miR-146b, pha-miR-148a, pha-miR-148b, pha-miR-151, pha-miR-152, pha-miR-155, pha-miR-16, pha-miR-181a, pha-miR-181b, pha-miR-181c, pha-miR-186, pha-miR-191, pha-miR-193a, pha-miR-193b, pha-miR-196a, pha-miR-199, pha-miR-19b, pha-miR-21, pha-miR-210, pha-miR-214, pha-miR-22, pha-miR-221, pha-miR-222, pha-miR-23a, pha-miR-23b, pha-miR-24, pha-miR-25, pha-miR-26a, pha-miR-26b, pha-miR-27a, pha-miR-27b, pha-miR-299, pha-miR-29a, pha-miR-30a, pha-miR-30c, pha-miR-30d, pha-miR-30e, pha-miR-31, pha-miR-320a, pha-miR-320b, pha-miR-340, pha-miR-361, pha-miR-378, pha-miR-381, pha-miR-409, pha-miR-411a, pha-miR-423, pha-miR-502b, pha-miR-532, pha-miR-582, pha-miR-660, pha-miR-708, pha-miR-760, pha-miR-769, pha-miR-877, pha-miR-887, pha-miR-92a, pha-miR-93, pha-miR-98, pha-miR-99a, pin-miR8788-3p, pin-miR8788-5p, pla-miR11598, pla-miR11599, pla-miR11600, pla-miR11601, pla-miR11602, pla-miR11603, pla-miR11604, pla-miR11605, pla-miR11606, pla-miR11607, pla-miR11608, pla-miR11609, pla-miR11610, pla-miR11611, pla-miR11612, pma-let-7a, pma-let-7b-3p, pma-let-7b-5p, pma-let-7c, pma-let-7d, pma-miR-100a-3p, pma-miR-100a-5p, pma-miR-100b, pma-miR-100c, pma-miR-103a, pma-miR-103b-3p, pma-miR-103b-5p, pma-miR-10a, pma-miR-10b-3p, pma-miR-10b-5p, pma-miR-124-3p, pma-miR-124-5p, pma-miR-125-3p, pma-miR-125-5p, pma-miR-126, pma-miR-128, pma-miR-129a-3p, pma-miR-129a-5p, pma-miR-130a-3p, pma-miR-130a-5p, pma-miR-130b, pma-miR-130c, pma-miR-130d, pma-miR-130e, pma-miR-132-3p, pma-miR-132-5p, pma-miR-1329, pma-miR-133a, pma-miR-133b-3p, pma-miR-133b-5p, pma-miR-135a-3p, pma-miR-135a-5p, pma-miR-135b, pma-miR-137-1-5p, pma-miR-137-2-5p, pma-miR-137-3p, pma-miR-138a, pma-miR-138b, pma-miR-140, pma-miR-143-3p, pma-miR-143-5p, pma-miR-144-3p, pma-miR-144-5p, pma-miR-145-3p, pma-miR-145-5p, pma-miR-146-3p, pma-miR-146-5p, pma-miR-147, pma-miR-152a, pma-miR-152b, pma-miR-153-3p, pma-miR-153-5p, pma-miR-155, pma-miR-15a, pma-miR-15b, pma-miR-16-3p, pma-miR-16-5p, pma-miR-1788a-3p, pma-miR-1788a-5p, pma-miR-1788b-3p, pma-miR-17a-3p, pma-miR-17a-5p, pma-miR-17b, pma-miR-17c, pma-miR-181a-3p, pma-miR-181a-5p, pma-miR-181b-3p, pma-miR-181b-5p, pma-miR-182, pma-miR-183-3p, pma-miR-183-5p, pma-miR-184, pma-miR-18a-3p, pma-miR-18a-5p, pma-miR-18b-3p, pma-miR-18b-5p, pma-miR-190a, pma-miR-190b-3p, pma-miR-190b-5p, pma-miR-192-3p, pma-miR-192-5p, pma-miR-193, pma-miR-194-3p, pma-miR-194-5p, pma-miR-196a-3p, pma-miR-196a-5p, pma-miR-196b, pma-miR-199a-3p, pma-miR-199a-5p, pma-miR-199b-3p, pma-miR-199b-5p, pma-miR-19a, pma-miR-19b-3p, pma-miR-19b-5p, pma-miR-19c, pma-miR-1a-3p, pma-miR-1a-5p, pma-miR-1b, pma-miR-1c-3p, pma-miR-1c-5p, pma-miR-1d, pma-miR-200b-3p, pma-miR-200b-5p, pma-miR-203a-3p, pma-miR-203a-5p, pma-miR-203b-3p, pma-miR-203b-5p, pma-miR-204-3p, pma-miR-204-5p, pma-miR-205a-3p, pma-miR-205a-5p, pma-miR-205b, pma-miR-208-3p, pma-miR-208-5p, pma-miR-20a-3p, pma-miR-20a-5p, pma-miR-20b, pma-miR-210, pma-miR-212-3p, pma-miR-212-5p, pma-miR-216a-3p, pma-miR-216a-5p, pma-miR-216b, pma-miR-217a, pma-miR-218a-3p, pma-miR-218a-5p, pma-miR-218b, pma-miR-221, pma-miR-222-3p, pma-miR-222-5p, pma-miR-22a-3p, pma-miR-22a-5p, pma-miR-22b-3p, pma-miR-22b-5p, pma-miR-23a-3p, pma-miR-23a-5p, pma-miR-23b, pma-miR-24, pma-miR-25b-3p, pma-miR-25b-5p, pma-miR-26a-1-3p, pma-miR-26a-2-3p, pma-miR-26a-5p, pma-miR-26b-3p, pma-miR-26b-5p, pma-miR-27a, pma-miR-27b-3p, pma-miR-27b-5p, pma-miR-281a, pma-miR-29a-3p, pma-miR-29a-5p, pma-miR-29b, pma-miR-29c, pma-miR-29d-3p, pma-miR-29d-5p, pma-miR-301a, pma-miR-301b, pma-miR-302, pma-miR-30a-3p, pma-miR-30a-5p, pma-miR-30b, pma-miR-30c-3p, pma-miR-30c-5p, pma-miR-30d, pma-miR-30e-3p, pma-miR-30e-5p, pma-miR-30f, pma-miR-30g, pma-miR-31, pma-miR-315, pma-miR-338b, pma-miR-33a, pma-miR-33b, pma-miR-375a-3p, pma-miR-375a-5p, pma-miR-375b-3p, pma-miR-375b-5p, pma-miR-429-3p, pma-miR-429-5p, pma-miR-430a, pma-miR-430b, pma-miR-430c, pma-miR-430d, pma-miR-430e, pma-miR-430f, pma-miR-451, pma-miR-4541, pma-miR-4542-3p, pma-miR-4542-5p, pma-miR-4543, pma-miR-4544-3p, pma-miR-4544-5p, pma-miR-4545-3p, pma-miR-4545-5p, pma-miR-4546, pma-miR-4547-3p, pma-miR-4547-5p, pma-miR-4548, pma-miR-4549, pma-miR-4550, pma-miR-4551, pma-miR-4552, pma-miR-4553, pma-miR-4554, pma-miR-4555, pma-miR-4556, pma-miR-4557, pma-miR-4558, pma-miR-4559, pma-miR-456, pma-miR-4560, pma-miR-4561, pma-miR-4562, pma-miR-4563, pma-miR-4564, pma-miR-4565, pma-miR-4566, pma-miR-4567, pma-miR-4568, pma-miR-4569, pma-miR-4570, pma-miR-4571-3p, pma-miR-4571-5p, pma-miR-4572, pma-miR-4573, pma-miR-4574, pma-miR-4575, pma-miR-4576, pma-miR-4577, pma-miR-4578, pma-miR-4579, pma-miR-4580, pma-miR-4581, pma-miR-4582, pma-miR-4583, pma-miR-4584, pma-miR-4585, pma-miR-4586, pma-miR-4587, pma-miR-4588, pma-miR-4589, pma-miR-459, pma-miR-4590, pma-miR-4591, pma-miR-4592, pma-miR-4593, pma-miR-4594, pma-miR-4595, pma-miR-4596, pma-miR-4597, pma-miR-4598, pma-miR-4599a, pma-miR-4599b, pma-miR-4600, pma-miR-4601, pma-miR-4602, pma-miR-4603, pma-miR-4604, pma-miR-4605, pma-miR-4606, pma-miR-4607, pma-miR-4608, pma-miR-4609, pma-miR-4610, pma-miR-4611, pma-miR-4612, pma-miR-4613, pma-miR-4614, pma-miR-4615, pma-miR-4616, pma-miR-4617, pma-miR-4618, pma-miR-4619, pma-miR-4620, pma-miR-4621, pma-miR-4622, pma-miR-4623, pma-miR-4624, pma-miR-4625, pma-miR-4626, pma-miR-4627, pma-miR-4628, pma-miR-4629, pma-miR-4630, pma-miR-4631, pma-miR-497-3p, pma-miR-497-5p, pma-miR-551, pma-miR-7a-3p, pma-miR-7a-5p, pma-miR-7b, pma-miR-875, pma-miR-92a, pma-miR-96a, pma-miR-9a-3p, pma-miR-9a-5p, pma-miR-9b, pmi-let-7-5p, pmi-miR-1-3p, pmi-miR-10-5p, pmi-miR-100-5p, pmi-miR-124-3p, pmi-miR-125-5p, pmi-miR-133-3p, pmi-miR-137-3p, pmi-miR-153-3p, pmi-miR-184-3p, pmi-miR-193-3p, pmi-miR-200-3p, pmi-miR-200-5p, pmi-miR-2001-5p, pmi-miR-2002-3p, pmi-miR-2004-3p, pmi-miR-2005-5p, pmi-miR-2006-3p, pmi-miR-2006-5p, pmi-miR-2007-5p, pmi-miR-2008-3p, pmi-miR-2009-3p, pmi-miR-2010-3p, pmi-miR-2011-3p, pmi-miR-2011-5p, pmi-miR-2012-5p, pmi-miR-2013-3p, pmi-miR-210-3p, pmi-miR-219-5p, pmi-miR-22-3p, pmi-miR-242-5p, pmi-miR-252a-5p, pmi-miR-252b-5p, pmi-miR-263-5p, pmi-miR-278-3p, pmi-miR-29a-3p, pmi-miR-29b-3p, pmi-miR-31-5p, pmi-miR-33-5p, pmi-miR-34-5p, pmi-miR-375-3p, pmi-miR-7-5p, pmi-miR-71-5p, pmi-miR-9-5p, pmi-miR-92a-3p, pmi-miR-92b-3p, pmi-miR-92c-3p, pmi-miR-92d-3p, pmi-miR-96-5p, pmi-miR-981-3p, pny-let-7a, pny-let-7b, pny-let-7c, pny-let-7d, pny-let-7e, pny-let-7f, pny-let-7g, pny-let-7h, pny-let-7i, pny-miR-1, pny-miR-100, pny-miR-101a, pny-miR-101b, pny-miR-103, pny-miR-10544, pny-miR-10549, pny-miR-10553, pny-miR-10555, pny-miR-10566, pny-miR-106, pny-miR-107, pny-miR-10a, pny-miR-10b, pny-miR-10c, pny-miR-10d, pny-miR-122, pny-miR-124, pny-miR-125a, pny-miR-125b, pny-miR-125c, pny-miR-126, pny-miR-128, pny-miR-129, pny-miR-130a, pny-miR-130b-3p, pny-miR-130b-5p, pny-miR-132, pny-miR-132-2, pny-miR-133, pny-miR-135a, pny-miR-135b, pny-miR-135c-3p, pny-miR-135c-5p, pny-miR-135d, pny-miR-137, pny-miR-138, pny-miR-1388, pny-miR-139, pny-miR-140, pny-miR-142, pny-miR-144, pny-miR-146, pny-miR-147, pny-miR-148-3p, pny-miR-148-5p, pny-miR-150, pny-miR-153a, pny-miR-153b, pny-miR-153c, pny-miR-155, pny-miR-15a, pny-miR-15b, pny-miR-15c-3p, pny-miR-15c-5p, pny-miR-16a, pny-miR-16b, pny-miR-17, pny-miR-181a, pny-miR-181b, pny-miR-182, pny-miR-183, pny-miR-184a, pny-miR-184b, pny-miR-187, pny-miR-18a, pny-miR-18b, pny-miR-190a, pny-miR-190b, pny-miR-192, pny-miR-194a, pny-miR-194b, pny-miR-196a, pny-miR-196b, pny-miR-199, pny-miR-19a, pny-miR-19b, pny-miR-19c, pny-miR-19d, pny-miR-200a, pny-miR-200b, pny-miR-203, pny-miR-204, pny-miR-205, pny-miR-206, pny-miR-20a, pny-miR-20b, pny-miR-21, pny-miR-212, pny-miR-212-2, pny-miR-214, pny-miR-216a, pny-miR-216b, pny-miR-2187a, pny-miR-2187b, pny-miR-2188, pny-miR-218a, pny-miR-218b, pny-miR-219a, pny-miR-219b, pny-miR-221, pny-miR-222, pny-miR-22a, pny-miR-22b, pny-miR-22c, pny-miR-23a, pny-miR-23b, pny-miR-23c, pny-miR-24a, pny-miR-24a-3, pny-miR-24a-4, pny-miR-24b, pny-miR-25, pny-miR-26a, pny-miR-26b, pny-miR-27a, pny-miR-27b, pny-miR-27d-3p, pny-miR-27d-5p, pny-miR-27e, pny-miR-29a, pny-miR-29b, pny-miR-301a, pny-miR-301b, pny-miR-301b-1, pny-miR-30a, pny-miR-30b, pny-miR-30c, pny-miR-30d, pny-miR-31, pny-miR-3120, pny-miR-33, pny-miR-338, pny-miR-34, pny-miR-365, pny-miR-375, pny-miR-429b, pny-miR-430a, pny-miR-430b, pny-miR-449a, pny-miR-455, pny-miR-456, pny-miR-457, pny-miR-458, pny-miR-460, pny-miR-462, pny-miR-489, pny-miR-499, pny-miR-551, pny-miR-7, pny-miR-7132a-3p, pny-miR-7132a-5p, pny-miR-7132b, pny-miR-7133, pny-miR-7147, pny-miR-722, pny-miR-723a, pny-miR-723b, pny-miR-724, pny-miR-725, pny-miR-726, pny-miR-728, pny-miR-729, pny-miR-730, pny-miR-731, pny-miR-734, pny-miR-8159, pny-miR-8160b, pny-miR-92, pny-miR-93, pny-miR-96, pny-miR-99a, pny-miR-99b, pol-let-7a-3p, pol-let-7a-5p, pol-let-7b-3p, pol-let-7b-5p, pol-let-7d-3p, pol-let-7d-5p, pol-miR-1-3p, pol-miR-1-5p, pol-miR-10b-3p, pol-miR-10b-5p, pol-miR-122-3p, pol-miR-122-5p, pol-miR-124-3p, pol-miR-124-5p, pol-miR-133-3p, pol-miR-133-5p, pol-miR-140-3p, pol-miR-140-5p, pol-miR-144-3p, pol-miR-144-5p, pol-miR-182-3p, pol-miR-182-5p, pol-miR-199a-3p, pol-miR-199a-5p, pol-miR-203-3p, pol-miR-203-5p, pol-miR-206-3p, pol-miR-206-5p, pol-miR-21-3p, pol-miR-21-5p, pol-miR-219-3p, pol-miR-219-5p, pol-miR-22-3p, pol-miR-22-5p, pol-miR-221-3p, pol-miR-221-5p, pol-miR-9b-3p, pol-miR-9b-5p, ppa-let-7a, ppa-let-7b, ppa-let-7c, ppa-let-7e, ppa-let-7f, ppa-let-7g, ppa-let-7i, ppa-miR-1, ppa-miR-100, ppa-miR-101, ppa-miR-103, ppa-miR-105, ppa-miR-106a, ppa-miR-106b, ppa-miR-107, ppa-miR-10a, ppa-miR-10b, ppa-miR-124a, ppa-miR-125a, ppa-miR-125b, ppa-miR-127, ppa-miR-128, ppa-miR-1304, ppa-miR-1307, ppa-miR-130a, ppa-miR-133a, ppa-miR-134, ppa-miR-135, ppa-miR-136, ppa-miR-139, ppa-miR-140, ppa-miR-141, ppa-miR-143, ppa-miR-144, ppa-miR-145, ppa-miR-146b, ppa-miR-147, ppa-miR-148a, ppa-miR-148b, ppa-miR-151, ppa-miR-152, ppa-miR-154, ppa-miR-155, ppa-miR-15a, ppa-miR-15b, ppa-miR-16, ppa-miR-17-3p, ppa-miR-17-5p, ppa-miR-18, ppa-miR-181a-3p, ppa-miR-181a-5p, ppa-miR-181b, ppa-miR-181c, ppa-miR-181d, ppa-miR-183, ppa-miR-186, ppa-miR-187, ppa-miR-188, ppa-miR-190, ppa-miR-191, ppa-miR-192, ppa-miR-193a, ppa-miR-193b, ppa-miR-195, ppa-miR-196, ppa-miR-197, ppa-miR-198, ppa-miR-199a, ppa-miR-199b, ppa-miR-19a, ppa-miR-19b, ppa-miR-20, ppa-miR-204, ppa-miR-205, ppa-miR-21, ppa-miR-210, ppa-miR-214, ppa-miR-216, ppa-miR-217, ppa-miR-218, ppa-miR-22, ppa-miR-221, ppa-miR-222, ppa-miR-223, ppa-miR-224, ppa-miR-2355, ppa-miR-23a, ppa-miR-23b, ppa-miR-24-3p, ppa-miR-24-5p, ppa-miR-25, ppa-miR-26a, ppa-miR-26b, ppa-miR-27a, ppa-miR-27b, ppa-miR-28, ppa-miR-299, ppa-miR-29a, ppa-miR-29b, ppa-miR-30a-3p, ppa-miR-30a-5p, ppa-miR-30b, ppa-miR-30d, ppa-miR-30e, ppa-miR-31, ppa-miR-32, ppa-miR-320a, ppa-miR-320b, ppa-miR-323, ppa-miR-328, ppa-miR-33, ppa-miR-340, ppa-miR-342, ppa-miR-34a, ppa-miR-34c, ppa-miR-369, ppa-miR-370, ppa-miR-376c, ppa-miR-378a, ppa-miR-379, ppa-miR-381, ppa-miR-382, ppa-miR-409, ppa-miR-410, ppa-miR-411a, ppa-miR-412, ppa-miR-421, ppa-miR-423, ppa-miR-424, ppa-miR-432, ppa-miR-450a, ppa-miR-484, ppa-miR-485, ppa-miR-486, ppa-miR-486-2, ppa-miR-493, ppa-miR-494, ppa-miR-532, ppa-miR-542, ppa-miR-549, ppa-miR-574, ppa-miR-615, ppa-miR-629, ppa-miR-654, ppa-miR-660, ppa-miR-665, ppa-miR-7, ppa-miR-769, ppa-miR-887, ppa-miR-889, ppa-miR-92a, ppa-miR-92b, ppa-miR-93, ppa-miR-95, ppa-miR-96, ppa-miR-98, ppa-miR-99a, ppc-let-7, ppc-lin-4, ppc-miR-1, ppc-miR-124, ppc-miR-133-3p, ppc-miR-133-5p, ppc-miR-2232, ppc-miR-2233, ppc-miR-2234a, ppc-miR-2234b, ppc-miR-2235a-10-3p, ppc-miR-2235a-11-3p, ppc-miR-2235a-3p, ppc-miR-2235a-5p, ppc-miR-2235a-9-3p, ppc-miR-2235b-3p, ppc-miR-2235b-5p, ppc-miR-2235c-3p, ppc-miR-2235c-5p, ppc-miR-2235d-3p, ppc-miR-2235d-5p, ppc-miR-2236a, ppc-miR-2236b, ppc-miR-2237a, ppc-miR-2237b, ppc-miR-2237c, ppc-miR-2237d-3p, ppc-miR-2237d-5p, ppc-miR-2238a, ppc-miR-2238b, ppc-miR-2238c, ppc-miR-2238d, ppc-miR-2238e, ppc-miR-2238f-3p, ppc-miR-2238f-5p, ppc-miR-2238g-3p, ppc-miR-2238g-5p, ppc-miR-2238 h-3p, ppc-miR-2238 h-5p, ppc-miR-2238i-3p, ppc-miR-2238i-5p, ppc-miR-2238j-3p, ppc-miR-2238j-5p, ppc-miR-2238k-3p, ppc-miR-2238k-5p, ppc-miR-22381-3p, ppc-miR-22381-5p, ppc-miR-2239, ppc-miR-2240a, ppc-miR-2240b, ppc-miR-2240c, ppc-miR-2241a, ppc-miR-2241b, ppc-miR-2241c, ppc-miR-2242, ppc-miR-2243, ppc-miR-2244, ppc-miR-2245, ppc-miR-2246, ppc-miR-2247, ppc-miR-2248, ppc-miR-2249, ppc-miR-2250, ppc-miR-2251a, ppc-miR-2251b-3p, ppc-miR-2251b-5p, ppc-miR-2252, ppc-miR-2253a, ppc-miR-2253b, ppc-miR-2253c-3p, ppc-miR-2253c-5p, ppc-miR-2253d-3p, ppc-miR-2253d-5p, ppc-miR-2254, ppc-miR-2255, ppc-miR-2256, ppc-miR-2258, ppc-miR-2259, ppc-miR-2260, ppc-miR-2261, ppc-miR-2262, ppc-miR-2263, ppc-miR-2264, ppc-miR-2265, ppc-miR-2266, ppc-miR-2267, ppc-miR-2268, ppc-miR-2269, ppc-miR-2270, ppc-miR-2271, ppc-miR-2272, ppc-miR-2273, ppc-miR-2274, ppc-miR-228-3p, ppc-miR-228-5p, ppc-miR-232, ppc-miR-234, ppc-miR-235-3p, ppc-miR-235-5p, ppc-miR-236, ppc-miR-239, ppc-miR-240, ppc-miR-242, ppc-miR-252, ppc-miR-279, ppc-miR-2a-3p, ppc-miR-2a-5p, ppc-miR-2b, ppc-miR-312a, ppc-miR-312b-3p, ppc-miR-312b-5p, ppc-miR-36-3p, ppc-miR-36-5p, ppc-miR-360-3p, ppc-miR-360-5p, ppc-miR-37, ppc-miR-38, ppc-miR-42a, ppc-miR-42b, ppc-miR-42c-3p, ppc-miR-42c-5p, ppc-miR-45, ppc-miR-46, ppc-miR-54, ppc-miR-55a, ppc-miR-55b, ppc-miR-56, ppc-miR-57-3p, ppc-miR-57-5p, ppc-miR-59-3p, ppc-miR-59-5p, ppc-miR-63a, ppc-miR-63b, ppc-miR-63c, ppc-miR-63d, ppc-miR-63e, ppc-miR-63f, ppc-miR-63g, ppc-miR-63h, ppc-miR-63i-3p, ppc-miR-63i-5p, ppc-miR-63j-3p, ppc-miR-63j-5p, ppc-miR-63k-3p, ppc-miR-63k-5p, ppc-miR-65, ppc-miR-67, ppc-miR-71a, ppc-miR-71b-3p, ppc-miR-71b-5p, ppc-miR-72, ppc-miR-76-3p, ppc-miR-76-5p, ppc-miR-79, ppc-miR-81, ppc-miR-8213-3p, ppc-miR-8213-5p, ppc-miR-8214-3p, ppc-miR-8214-5p, ppc-miR-8215a-3p, ppc-miR-8215a-5p, ppc-miR-8215b-3p, ppc-miR-8215b-5p, ppc-miR-8216-3p, ppc-miR-8216-5p, ppc-miR-8217-3p, ppc-miR-8217-5p, ppc-miR-8218a-3p, ppc-miR-8218a-5p, ppc-miR-8218b-3p, ppc-miR-8218b-5p, ppc-miR-8219-3p, ppc-miR-8219-5p, ppc-miR-8220-3p, ppc-miR-8220-5p, ppc-miR-8221-3p, ppc-miR-8221-5p, ppc-miR-8222-3p, ppc-miR-8222-5p, ppc-miR-8223-3p, ppc-miR-8223-5p, ppc-miR-8224-3p, ppc-miR-8224-5p, ppc-miR-8225-3p, ppc-miR-8225-5p, ppc-miR-8226-3p, ppc-miR-8226-5p, ppc-miR-8227-3p, ppc-miR-8227-5p, ppc-miR-8228-3p, ppc-miR-8228-5p, ppc-miR-8229a-3p, ppc-miR-8229a-5p, ppc-miR-8229b-3p, ppc-miR-8229b-5p, ppc-miR-8230-3p, ppc-miR-8230-5p, ppc-miR-8231-3p, ppc-miR-8231-5p, ppc-miR-8232-3p, ppc-miR-8232-5p, ppc-miR-8233-3p, ppc-miR-8233-5p, ppc-miR-8234-3p, ppc-miR-8234-5p, ppc-miR-8235-3p, ppc-miR-8235-5p, ppc-miR-8236-3p, ppc-miR-8236-5p, ppc-miR-8237-3p, ppc-miR-8237-5p, ppc-miR-8238-3p, ppc-miR-8238-5p, ppc-miR-8239-3p, ppc-miR-8239-5p, ppc-miR-8240-3p, ppc-miR-8240-5p, ppc-miR-8241-3p, ppc-miR-8241-5p, ppc-miR-8242-3p, ppc-miR-8242-5p, ppc-miR-8243-3p, ppc-miR-8243-5p, ppc-miR-8244-3p, ppc-miR-8244-5p, ppc-miR-8245a-3p, ppc-miR-8245a-5p, ppc-miR-8245b-3p, ppc-miR-8245b-5p, ppc-miR-8246-3p, ppc-miR-8246-5p, ppc-miR-8247-3p, ppc-miR-8247-5p, ppc-miR-8248-3p, ppc-miR-8248-5p, ppc-miR-8249-3p, ppc-miR-8249-5p, ppc-miR-8250a-2-3p, ppc-miR-8250a-3p, ppc-miR-8250a-5p, ppc-miR-8250c-3p, ppc-miR-8250c-5p, ppc-miR-8250d-3p, ppc-miR-8250d-5p, ppc-miR-8251-3p, ppc-miR-8251-5p, ppc-miR-8252a-3p, ppc-miR-8252a-5p, ppc-miR-8252b-3p, ppc-miR-8252b-5p, ppc-miR-8253-3p, ppc-miR-8253-5p, ppc-miR-8254-3p, ppc-miR-8254-5p, ppc-miR-8255-3p, ppc-miR-8255-5p, ppc-miR-8256-3p, ppc-miR-8256-5p, ppc-miR-8257-3p, ppc-miR-8257-5p, ppc-miR-8258-3p, ppc-miR-8258-5p, ppc-miR-8259-3p, ppc-miR-8259-5p, ppc-miR-8260-3p, ppc-miR-8260-5p, ppc-miR-8261-3p, ppc-miR-8261-5p, ppc-miR-8262-3p, ppc-miR-8262-5p, ppc-miR-8263-3p, ppc-miR-8263-5p, ppc-miR-8264-3p, ppc-miR-8264-5p, ppc-miR-8265-3p, ppc-miR-8265-5p, ppc-miR-8266-3p, ppc-miR-8266-5p, ppc-miR-8267-3p, ppc-miR-8267-5p, ppc-miR-8268-3p, ppc-miR-8268-5p, ppc-miR-8269-3p, ppc-miR-8269-5p, ppc-miR-8270-3p, ppc-miR-8270-5p, ppc-miR-8271-3p, ppc-miR-8271-5p, ppc-miR-8272-3p, ppc-miR-8272-5p, ppc-miR-8273-3p, ppc-miR-8273-5p, ppc-miR-8274-3p, ppc-miR-8274-5p, ppc-miR-8275-3p, ppc-miR-8275-5p, ppc-miR-8276-3p, ppc-miR-8276-5p, ppc-miR-8277-3p, ppc-miR-8277-5p, ppc-miR-8278-3p, ppc-miR-8278-5p, ppc-miR-8279-3p, ppc-miR-8279-5p, ppc-miR-8280-3p, ppc-miR-8280-5p, ppc-miR-8281-3p, ppc-miR-8281-5p, ppc-miR-8282a-3p, ppc-miR-8282a-5p, ppc-miR-8282b-3p, ppc-miR-8282b-5p, ppc-miR-8282c-3p, ppc-miR-8282c-5p, ppc-miR-8283-3p, ppc-miR-8283-5p, ppc-miR-8284-3p, ppc-miR-8284-5p, ppc-miR-8285-3p, ppc-miR-8285-5p, ppc-miR-8286a-3p, ppc-miR-8286a-5p, ppc-miR-8286b-3p, ppc-miR-8286b-5p, ppc-miR-8287-3p, ppc-miR-8287-5p, ppc-miR-8288-3p, ppc-miR-8288-5p, ppc-miR-8289-3p, ppc-miR-8289-5p, ppc-miR-8290-3p, ppc-miR-8290-5p, ppc-miR-8291-3p, ppc-miR-8291-5p, ppc-miR-8292-3p, ppc-miR-8292-5p, ppc-miR-8293-3p, ppc-miR-8293-5p, ppc-miR-8294-3p, ppc-miR-8294-5p, ppc-miR-8295-3p, ppc-miR-8295-5p, ppc-miR-8296-3p, ppc-miR-8296-5p, ppc-miR-8297-3p, ppc-miR-8297-5p, ppc-miR-8298-3p, ppc-miR-8298-5p, ppc-miR-8299-3p, ppc-miR-8299-5p, ppc-miR-83-3p, ppc-miR-83-5p, ppc-miR-8300-3p, ppc-miR-8300-5p, ppc-miR-8301-3p, ppc-miR-8301-5p, ppc-miR-8302-3p, ppc-miR-8302-5p, ppc-miR-8303-2-3p, ppc-miR-8303-3p, ppc-miR-8303-5p, ppc-miR-8304a-3p, ppc-miR-8304a-5p, ppc-miR-8304b-3p, ppc-miR-8304b-5p, ppc-miR-8305-3p, ppc-miR-8305-5p, ppc-miR-8306-3p, ppc-miR-8306-5p, ppc-miR-8307-3p, ppc-miR-8307-5p, ppc-miR-8308-3p, ppc-miR-8308-5p, ppc-miR-8309-3p, ppc-miR-8309-5p, ppc-miR-8310-3p, ppc-miR-8310-5p, ppc-miR-8311-3p, ppc-miR-8311-5p, ppc-miR-8312-3p, ppc-miR-8312-5p, ppc-miR-8313-3p, ppc-miR-8313-5p, ppc-miR-8314-3p, ppc-miR-8314-5p, ppc-miR-8315-2-5p, ppc-miR-8315-3p, ppc-miR-8315-5p, ppc-miR-8316-3p, ppc-miR-8316-5p, ppc-miR-8317a-3p, ppc-miR-8317a-5p, ppc-miR-8317b-3p, ppc-miR-8317b-5p, ppc-miR-8318-3p, ppc-miR-8318-5p, ppc-miR-8319-3p, ppc-miR-8319-5p, ppc-miR-8320-3p, ppc-miR-8320-5p, ppc-miR-8321-3p, ppc-miR-8321-5p, ppc-miR-8322-3p, ppc-miR-8322-5p, ppc-miR-8323-3p, ppc-miR-8323-5p, ppc-miR-8324-3p, ppc-miR-8324-5p, ppc-miR-8325-3p, ppc-miR-8325-5p, ppc-miR-8326-3p, ppc-miR-8326-5p, ppc-miR-8327-3p, ppc-miR-8327-5p, ppc-miR-8328-3p, ppc-miR-8328-5p, ppc-miR-8329-3p, ppc-miR-8329-5p, ppc-miR-8330-3p, ppc-miR-8330-5p, ppc-miR-8331-3p, ppc-miR-8331-5p, ppc-miR-8332-3p, ppc-miR-8332-5p, ppc-miR-8333-3p, ppc-miR-8333-5p, ppc-miR-8334-3p, ppc-miR-8334-5p, ppc-miR-8335-3p, ppc-miR-8335-5p, ppc-miR-8336-3p, ppc-miR-8336-5p, ppc-miR-8337-3p, ppc-miR-8337-5p, ppc-miR-8338-2-3p, ppc-miR-8338-3p, ppc-miR-8338-5p, ppc-miR-8339-3p, ppc-miR-8339-5p, ppc-miR-8340-3p, ppc-miR-8340-5p, ppc-miR-8341-3p, ppc-miR-8341-5p, ppc-miR-8342-3p, ppc-miR-8342-5p, ppc-miR-8343-3p, ppc-miR-8343-5p, ppc-miR-8344-3p, ppc-miR-8344-5p, ppc-miR-8345-3p, ppc-miR-8345-5p, ppc-miR-8346-3p, ppc-miR-8346-5p, ppc-miR-8347-3p, ppc-miR-8347-5p, ppc-miR-8348-3p, ppc-miR-8348-5p, ppc-miR-8349-3p, ppc-miR-8349-5p, ppc-miR-8350-3p, ppc-miR-8350-5p, ppc-miR-8351-3p, ppc-miR-8351-5p, ppc-miR-8352-3p, ppc-miR-8352-5p, ppc-miR-8353-3p, ppc-miR-8353-5p, ppc-miR-8354-3p, ppc-miR-8354-5p, ppc-miR-8355-3p, ppc-miR-8355-5p, ppc-miR-8356a-3p, ppc-miR-8356a-5p, ppc-miR-8356b-3p, ppc-miR-8356b-5p, ppc-miR-8357-3p, ppc-miR-8357-5p, ppc-miR-8358-3p, ppc-miR-8358-5p, ppc-miR-8359-3p, ppc-miR-8359-5p, ppc-miR-8360-3p, ppc-miR-8360-5p, ppc-miR-8361-3p, ppc-miR-8361-5p, ppc-miR-8362-3p, ppc-miR-8362-5p, ppc-miR-8363-3p, ppc-miR-8363-5p, ppc-miR-8364a-3p, ppc-miR-8364a-5p, ppc-miR-8364b-3p, ppc-miR-8364b-5p, ppc-miR-8364c-2-3p, ppc-miR-8364c-3p, ppc-miR-8364c-5p, ppc-miR-8364d-3p, ppc-miR-8364d-5p, ppc-miR-8364e-3p, ppc-miR-8364e-5p, ppc-miR-8364f-3p, ppc-miR-8364f-5p, ppc-miR-8364g-3p, ppc-miR-8364g-5p, ppc-miR-8364 h-3p, ppc-miR-8364 h-5p, ppc-miR-8364i-3p, ppc-miR-8364i-5p, ppc-miR-8364j-3p, ppc-miR-8364j-5p, ppc-miR-8364k-3p, ppc-miR-8364k-5p, ppc-miR-83641-3p, ppc-miR-83641-5p, ppc-miR-8364m-3p, ppc-miR-8364m-5p, ppc-miR-8364n-3p, ppc-miR-8364n-5p, ppc-miR-83640-3p, ppc-miR-83640-5p, ppc-miR-8364p-3p, ppc-miR-8364p-5p, ppc-miR-8364q-3p, ppc-miR-8364q-5p, ppc-miR-8364r-3p, ppc-miR-8364r-5p, ppc-miR-8364s-3p, ppc-miR-8364s-5p, ppc-miR-8364t-3p, ppc-miR-8364t-5p, ppc-miR-8364u-3p, ppc-miR-8364u-5p, ppc-miR-8364v-3p, ppc-miR-8364v-5p, ppc-miR-8364w-3p, ppc-miR-8364w-5p, ppc-miR-84a, ppc-miR-84b, ppc-miR-84c-3p, ppc-miR-84c-5p, ppc-miR-84d-3p, ppc-miR-84d-5p, ppc-miR-84e-3p, ppc-miR-84e-5p, ppc-miR-84f-3p, ppc-miR-84f-5p, ppc-miR-86, ppc-miR-87, ppc-miR-9, ppc-miR-993, ppe-miR1511-3p, ppe-miR1511-5p, ppe-miR156a, ppe-miR156b, ppe-miR156c, ppe-miR156d, ppe-miR156e, ppe-miR156f, ppe-miR156g, ppe-miR156h, ppe-miR156i, ppe-miR159, ppe-miR160a, ppe-miR160b, ppe-miR162, ppe-miR164a, ppe-miR164b, ppe-miR164c, ppe-miR164d, ppe-miR166a, ppe-miR166b, ppe-miR166c, ppe-miR166d, ppe-miR166e, ppe-miR167a, ppe-miR167b, ppe-miR167c, ppe-miR167d, ppe-miR168, ppe-miR169a, ppe-miR169b, ppe-miR169c, ppe-miR169d, ppe-miR169e-5p, ppe-miR169f, ppe-miR169g, ppe-miR169h, ppe-miR169i, ppe-miR169j, ppe-miR169k, ppe-miR1691, ppe-miR171a, ppe-miR171b, ppe-miR171c, ppe-miR171d-3p, ppe-miR171d-5p, ppe-miR171e, ppe-miR171f, ppe-miR171g, ppe-miR171h, ppe-miR172a-3p, ppe-miR172a-5p, ppe-miR172b, ppe-miR172c, ppe-miR172d, ppe-miR2111a, ppe-miR2111b, ppe-miR2111c, ppe-miR2111d, ppe-miR319a, ppe-miR319b, ppe-miR3627-3p, ppe-miR3627-5p, ppe-miR390, ppe-miR393a, ppe-miR393b, ppe-miR394a, ppe-miR394b, ppe-miR395a-3p, ppe-miR395a-5p, ppe-miR395b-3p, ppe-miR395b-5p, ppe-miR395c, ppe-miR395d, ppe-miR395e, ppe-miR395f, ppe-miR395g, ppe-miR395h, ppe-miR395i, ppe-miR395j, ppe-miR395k, ppe-miR3951, ppe-miR395m, ppe-miR395n, ppe-miR3950, ppe-miR396a, ppe-miR396b, ppe-miR397, ppe-miR398a-3p, ppe-miR398a-5p, ppe-miR398b, ppe-miR399a, ppe-miR399b, ppe-miR399c, ppe-miR399d, ppe-miR399e, ppe-miR399f, ppe-miR399g, ppe-miR399h, ppe-miR399i, ppe-miR399j, ppe-miR399k, ppe-miR3991, ppe-miR399m, ppe-miR399n, ppe-miR403, ppe-miR477-3p, ppe-miR477-5p, ppe-miR477a-3p, ppe-miR477a-5p, ppe-miR477b-3p, ppe-miR477b-5p, ppe-miR482a-3p, ppe-miR482a-5p, ppe-miR482b-3p, ppe-miR482b-5p, ppe-miR482c-3p, ppe-miR482c-5p, ppe-miR482d-3p, ppe-miR482d-5p, ppe-miR482e, ppe-miR482f, ppe-miR5225-3p, ppe-miR5225-5p, ppe-miR530, ppe-miR535a, ppe-miR535b, ppe-miR6257, ppe-miR6258, ppe-miR6259, ppe-miR6260, ppe-miR6261, ppe-miR6262, ppe-miR6263, ppe-miR6264, ppe-miR6265, ppe-miR6266a, ppe-miR6266b, ppe-miR6266c, ppe-miR6267a, ppe-miR6267b, ppe-miR6267c-3p, ppe-miR6267c-5p, ppe-miR6269, ppe-miR6270, ppe-miR6271, ppe-miR6272, ppe-miR6273, ppe-miR6274a, ppe-miR6274b-3p, ppe-miR6274b-5p, ppe-miR6275, ppe-miR6276, ppe-miR6277, ppe-miR6278, ppe-miR6279, ppe-miR6280, ppe-miR6281, ppe-miR6282, ppe-miR6283, ppe-miR6284, ppe-miR6285, ppe-miR6286, ppe-miR6287, ppe-miR6288a, ppe-miR6288b-3p, ppe-miR6288b-5p, ppe-miR6288c-3p, ppe-miR6288c-5p, ppe-miR6289, ppe-miR6290, ppe-miR6291a, ppe-miR6291b, ppe-miR6291c-3p, ppe-miR6291c-5p, ppe-miR6292, ppe-miR6293, ppe-miR6294, ppe-miR6295, ppe-miR6296, ppe-miR6297a, ppe-miR6297b, ppe-miR7122a-3p, ppe-miR7122a-5p, ppe-miR7122b-3p, ppe-miR7122b-5p, ppe-miR7125-3p, ppe-miR7125-5p, ppe-miR8122-3p, ppe-miR8122-5p, ppe-miR8123-3p, ppe-miR8123-5p, ppe-miR8124-3p, ppe-miR8124-5p, ppe-miR8125, ppe-miR8126-3p, ppe-miR8126-5p, ppe-miR8127-3p, ppe-miR8127-5p, ppe-miR8128-3p, ppe-miR8128-5p, ppe-miR8129-3p, ppe-miR8129-5p, ppe-miR8130-3p, ppe-miR8130-5p, ppe-miR8131-3p, ppe-miR8131-5p, ppe-miR8132, ppe-miR8133-3p, ppe-miR8133-5p, ppe-miR827, ppe-miR828-3p, ppe-miR828-5p, ppe-miR858, ppt-miR1023a-3p, ppt-miR1023a-5p, ppt-miR1023b-3p, ppt-miR1023b-5p, ppt-miR1023c-3p, ppt-miR1023c-5p, ppt-miR1023d-5p, ppt-miR1023e-3p, ppt-miR1023e-5p, ppt-miR1024a, ppt-miR1024b, ppt-miR1025, ppt-miR1026a, ppt-miR1026b, ppt-miR1027a, ppt-miR1027b, ppt-miR1027c, ppt-miR1028a-3p, ppt-miR1028a-5p, ppt-miR1028b-3p, ppt-miR1028b-5p, ppt-miR1028c-3p, ppt-miR1028c-5p, ppt-miR1029, ppt-miR1030a, ppt-miR1030b, ppt-miR1030c, ppt-miR1030d, ppt-miR1030e, ppt-miR1030f, ppt-miR1030g, ppt-miR1030h, ppt-miR1030i, ppt-miR1030j, ppt-miR1031a, ppt-miR1031b, ppt-miR1032, ppt-miR1033a, ppt-miR1033b, ppt-miR1033c, ppt-miR1033d, ppt-miR1033e, ppt-miR1034, ppt-miR1034b, ppt-miR1035, ppt-miR1036-3p, ppt-miR1036-5p, ppt-miR1037, ppt-miR1038-3p, ppt-miR1038-5p, ppt-miR1039-3p, ppt-miR1039-5p, ppt-miR1040, ppt-miR1041, ppt-miR1042-3p, ppt-miR1042-5p, ppt-miR1043-3p, ppt-miR1043-5p, ppt-miR1044-3p, ppt-miR1044-5p, ppt-miR1045, ppt-miR1046-3p, ppt-miR1046-5p, ppt-miR1047-3p, ppt-miR1047-5p, ppt-miR1048-3p, ppt-miR1048-5p, ppt-miR1049, ppt-miR1049b, ppt-miR1050, ppt-miR1051-3p, ppt-miR1051-5p, ppt-miR1052, ppt-miR1053-3p, ppt-miR1053-5p, ppt-miR1054, ppt-miR1055, ppt-miR1056, ppt-miR1057, ppt-miR1058, ppt-miR1059, ppt-miR1060, ppt-miR1061-3p, ppt-miR1061-5p, ppt-miR1062, ppt-miR1063a, ppt-miR1063b, ppt-miR1063c, ppt-miR1063d, ppt-miR1063e, ppt-miR1063f, ppt-miR1063g, ppt-miR1063h, ppt-miR1064-3p, ppt-miR1064-5p, ppt-miR1065, ppt-miR1066, ppt-miR1067, ppt-miR1068, ppt-miR1069-3p, ppt-miR1069-5p, ppt-miR1070, ppt-miR1071-3p, ppt-miR1071-5p, ppt-miR1072, ppt-miR1073-3p, ppt-miR1073-5p, ppt-miR1074, ppt-miR1075, ppt-miR1076-3p, ppt-miR1076-5p, ppt-miR1077-3p, ppt-miR1077-5p, ppt-miR1078, ppt-miR1079-3p, ppt-miR1079-5p, ppt-miR11316, ppt-miR11317, ppt-miR11318, ppt-miR11319, ppt-miR11320, ppt-miR11321, ppt-miR11322, ppt-miR11323, ppt-miR11324, ppt-miR11325, ppt-miR11326, ppt-miR11327, ppt-miR11328, ppt-miR11329, ppt-miR11330, ppt-miR1211-3p, ppt-miR1211-5p, ppt-miR1212, ppt-miR1214, ppt-miR1215, ppt-miR1216, ppt-miR1217-3p, ppt-miR1217-5p, ppt-miR1218, ppt-miR1219a, ppt-miR1219b, ppt-miR1219c, ppt-miR1219d, ppt-miR1220a, ppt-miR1220b, ppt-miR1221-3p, ppt-miR1221-5p, ppt-miR1222a, ppt-miR1222b, ppt-miR1222c, ppt-miR1222d, ppt-miR1222e, ppt-miR1223a, ppt-miR1223b, ppt-miR1223c, ppt-miR1223d, ppt-miR1223e, ppt-miR1223f, ppt-miR1223g, ppt-miR1223h, ppt-miR1223i, ppt-miR1223j, ppt-miR156a, ppt-miR156b, ppt-miR156c, ppt-miR160a, ppt-miR160b, ppt-miR160c, ppt-miR160d, ppt-miR160e, ppt-miR160f, ppt-miR160g, ppt-miR160h, ppt-miR160i, ppt-miR166a, ppt-miR166b, ppt-miR166c, ppt-miR166d, ppt-miR166e, ppt-miR166f, ppt-miR166g, ppt-miR166h, ppt-miR166i, ppt-miR166j, ppt-miR166k, ppt-miR1661, ppt-miR166m, ppt-miR167, ppt-miR171a, ppt-miR171b, ppt-miR2077, ppt-miR2078, ppt-miR2079, ppt-miR2080, ppt-miR2081, ppt-miR2082, ppt-miR2083-3p, ppt-miR2083-5p, ppt-miR2084, ppt-miR2085, ppt-miR319a, ppt-miR319b, ppt-miR319c, ppt-miR319d-3p, ppt-miR319d-5p.1, ppt-miR319d-5p.2, ppt-miR319e, ppt-miR390a, ppt-miR390b, ppt-miR390c-3p, ppt-miR390c-5p, ppt-miR395, ppt-miR408a, ppt-miR408b, ppt-miR414, ppt-miR419, ppt-miR477a-3p, ppt-miR477a-5p, ppt-miR477b, ppt-miR477c, ppt-miR477d, ppt-miR477e, ppt-miR477f, ppt-miR477g-3p, ppt-miR477g-5p, ppt-miR477h, ppt-miR529a, ppt-miR529b, ppt-miR529c, ppt-miR529d, ppt-miR529e, ppt-miR529f, ppt-miR529g, ppt-miR533a-3p, ppt-miR533a-5p, ppt-miR533b-3p, ppt-miR533b-5p, ppt-miR533c, ppt-miR533d, ppt-miR533e, ppt-miR534a, ppt-miR534b, ppt-miR535a, ppt-miR535b, ppt-miR535c, ppt-miR535d, ppt-miR536a, ppt-miR536b, ppt-miR536c, ppt-miR536d, ppt-miR536e, ppt-miR536f, ppt-miR537a, ppt-miR537b, ppt-miR537c, ppt-miR537d, ppt-miR538a, ppt-miR538b, ppt-miR538c, ppt-miR893, ppt-miR894, ppt-miR895, ppt-miR897, ppt-miR898a-3p, ppt-miR898a-5p, ppt-miR898b, ppt-miR899, ppt-miR900-3p, ppt-miR900-5p, ppt-miR901, ppt-miR902a-3p, ppt-miR902a-5p, ppt-miR902b-3p, ppt-miR902b-5p, ppt-miR902c-3p, ppt-miR902c-5p, ppt-miR902d-3p, ppt-miR902d-5p, ppt-miR902e-3p, ppt-miR902e-5p, ppt-miR902f-3p, ppt-miR902f-5p, ppt-miR902g-3p, ppt-miR902g-5p, ppt-miR902 h-3p, ppt-miR902 h-5p, ppt-miR902i-3p, ppt-miR902i-5p, ppt-miR902j-3p, ppt-miR902j-5p, ppt-miR902k-3p, ppt-miR902k-5p, ppt-miR9021-3p, ppt-miR9021-5p, ppt-miR903, ppt-miR904a, ppt-miR904b, ppy-let-7a, ppy-let-7b, ppy-let-7c, ppy-let-7d, ppy-let-7e, ppy-let-7f, ppy-let-7g, ppy-let-7i, ppy-miR-1, ppy-miR-100, ppy-miR-101, ppy-miR-103, ppy-miR-105, ppy-miR-106a, ppy-miR-106b, ppy-miR-107, ppy-miR-10a, ppy-miR-10b, ppy-miR-1178, ppy-miR-1179, ppy-miR-1180, ppy-miR-1181, ppy-miR-1183, ppy-miR-1185, ppy-miR-1193, ppy-miR-1197, ppy-miR-1199-3p, ppy-miR-1199-5p, ppy-miR-1200, ppy-miR-1202, ppy-miR-1204, ppy-miR-1205, ppy-miR-1207-3p, ppy-miR-1207-5p, ppy-miR-1208, ppy-miR-122, ppy-miR-1224-3p, ppy-miR-1224-5p, ppy-miR-1225-3p, ppy-miR-1225-5p, ppy-miR-1226, ppy-miR-1227, ppy-miR-1228, ppy-miR-1233, ppy-miR-1236, ppy-miR-1237, ppy-miR-1238, ppy-miR-124, ppy-miR-1244, ppy-miR-1245, ppy-miR-1246, ppy-miR-1247, ppy-miR-1248, ppy-miR-1249, ppy-miR-124a, ppy-miR-1250, ppy-miR-1251, ppy-miR-1252, ppy-miR-1253, ppy-miR-1255a, ppy-miR-1255b, ppy-miR-1256, ppy-miR-1257, ppy-miR-125a-3p, ppy-miR-125a-5p, ppy-miR-125b, ppy-miR-126, ppy-miR-1260a, ppy-miR-1260b, ppy-miR-1261, ppy-miR-1262, ppy-miR-1263, ppy-miR-1264, ppy-miR-1265, ppy-miR-1266, ppy-miR-1267, ppy-miR-1268, ppy-miR-1269, ppy-miR-127, ppy-miR-1270, ppy-miR-1271, ppy-miR-1275, ppy-miR-1277, ppy-miR-128, ppy-miR-1281, ppy-miR-1282, ppy-miR-1283a, ppy-miR-1284, ppy-miR-1285a, ppy-miR-1285b, ppy-miR-1286, ppy-miR-1287, ppy-miR-1289, ppy-miR-129-1-3p, ppy-miR-129-2-3p, ppy-miR-129-5p, ppy-miR-1290, ppy-miR-1291, ppy-miR-1292, ppy-miR-1293, ppy-miR-1295, ppy-miR-1296, ppy-miR-1297, ppy-miR-1298, ppy-miR-1299, ppy-miR-1301, ppy-miR-1302, ppy-miR-1303, ppy-miR-1304, ppy-miR-1305, ppy-miR-1306, ppy-miR-1307, ppy-miR-130a, ppy-miR-130b, ppy-miR-132, ppy-miR-1322, ppy-miR-1323, ppy-miR-1324, ppy-miR-133a, ppy-miR-133b, ppy-miR-133c, ppy-miR-134, ppy-miR-135a, ppy-miR-135b, ppy-miR-136, ppy-miR-137, ppy-miR-138, ppy-miR-139-3p, ppy-miR-139-5p, ppy-miR-140-3p, ppy-miR-140-5p, ppy-miR-141, ppy-miR-142-3p, ppy-miR-142-5p, ppy-miR-143, ppy-miR-144, ppy-miR-145, ppy-miR-1468, ppy-miR-1469, ppy-miR-146a, ppy-miR-146b-3p, ppy-miR-146b-5p, ppy-miR-1471, ppy-miR-147a, ppy-miR-147b, ppy-miR-148a, ppy-miR-148b, ppy-miR-149, ppy-miR-150, ppy-miR-151a-3p, ppy-miR-151a-5p, ppy-miR-151b, ppy-miR-152, ppy-miR-153, ppy-miR-1537, ppy-miR-1538, ppy-miR-154, ppy-miR-155, ppy-miR-15a, ppy-miR-15b, ppy-miR-16, ppy-miR-17-3p, ppy-miR-17-5p, ppy-miR-181a-3p, ppy-miR-181a-5p, ppy-miR-181b, ppy-miR-181c, ppy-miR-181d, ppy-miR-182, ppy-miR-1827, ppy-miR-183, ppy-miR-184, ppy-miR-185, ppy-miR-186, ppy-miR-187, ppy-miR-188, ppy-miR-18a, ppy-miR-18b, ppy-miR-1908, ppy-miR-1909, ppy-miR-190a, ppy-miR-190b, ppy-miR-191, ppy-miR-1910, ppy-miR-1912, ppy-miR-1913, ppy-miR-1914, ppy-miR-1915, ppy-miR-192, ppy-miR-193a-3p, ppy-miR-193a-5p, ppy-miR-193b, ppy-miR-194, ppy-miR-195, ppy-miR-196, ppy-miR-196-2, ppy-miR-196b, ppy-miR-197, ppy-miR-1976, ppy-miR-198, ppy-miR-199a, ppy-miR-199b-3p, ppy-miR-199b-5p, ppy-miR-19a, ppy-miR-19b, ppy-miR-200a, ppy-miR-200c, ppy-miR-202, ppy-miR-203, ppy-miR-204, ppy-miR-205, ppy-miR-206, ppy-miR-208a, ppy-miR-208b, ppy-miR-20a, ppy-miR-20b, ppy-miR-21, ppy-miR-210, ppy-miR-211, ppy-miR-214, ppy-miR-215, ppy-miR-216a, ppy-miR-216b, ppy-miR-217, ppy-miR-218, ppy-miR-219, ppy-miR-219-5p, ppy-miR-22, ppy-miR-221, ppy-miR-222, ppy-miR-223, ppy-miR-224, ppy-miR-2278, ppy-miR-2355, ppy-miR-23a, ppy-miR-23b, ppy-miR-24-3p, ppy-miR-24-5p, ppy-miR-25, ppy-miR-26a, ppy-miR-26b, ppy-miR-27a, ppy-miR-27b, ppy-miR-28, ppy-miR-2861, ppy-miR-296-3p, ppy-miR-296-5p, ppy-miR-297, ppy-miR-298, ppy-miR-299-3p, ppy-miR-299-5p, ppy-miR-29a, ppy-miR-29b, ppy-miR-29c, ppy-miR-300, ppy-miR-301a, ppy-miR-301b, ppy-miR-302b, ppy-miR-302c, ppy-miR-302d, ppy-miR-302e, ppy-miR-30a-3p, ppy-miR-30a-5p, ppy-miR-30b, ppy-miR-30c, ppy-miR-30d, ppy-miR-30e, ppy-miR-31, ppy-miR-3121, ppy-miR-3136, ppy-miR-3145, ppy-miR-3154, ppy-miR-3155a, ppy-miR-3170, ppy-miR-3173, ppy-miR-3174, ppy-miR-3188, ppy-miR-32, ppy-miR-320a, ppy-miR-320b, ppy-miR-320c, ppy-miR-320d, ppy-miR-320e, ppy-miR-323-3p, ppy-miR-323-5p, ppy-miR-324-3p, ppy-miR-324-5p, ppy-miR-325, ppy-miR-326, ppy-miR-328, ppy-miR-329, ppy-miR-330-3p, ppy-miR-330-5p, ppy-miR-331-3p, ppy-miR-331-5p, ppy-miR-335, ppy-miR-337-3p, ppy-miR-337-5p, ppy-miR-338-3p, ppy-miR-338-5p, ppy-miR-339-5p, ppy-miR-33a, ppy-miR-33b, ppy-miR-340, ppy-miR-342-3p, ppy-miR-342-5p, ppy-miR-345, ppy-miR-346, ppy-miR-34a, ppy-miR-34b, ppy-miR-34c-3p, ppy-miR-34c-5p, ppy-miR-361-3p, ppy-miR-361-5p, ppy-miR-3617, ppy-miR-362-3p, ppy-miR-362-5p, ppy-miR-363, ppy-miR-365, ppy-miR-3660, ppy-miR-3661, ppy-miR-367, ppy-miR-369-3p, ppy-miR-369-5p, ppy-miR-370, ppy-miR-371-3p, ppy-miR-371-5p, ppy-miR-372, ppy-miR-373, ppy-miR-374a, ppy-miR-375, ppy-miR-376a, ppy-miR-376b, ppy-miR-376c, ppy-miR-377, ppy-miR-378a, ppy-miR-378b, ppy-miR-378d, ppy-miR-378e, ppy-miR-379, ppy-miR-380, ppy-miR-381, ppy-miR-382, ppy-miR-383, ppy-miR-384, ppy-miR-3912, ppy-miR-3934, ppy-miR-3938, ppy-miR-3940, ppy-miR-3943, ppy-miR-409-3p, ppy-miR-409-5p, ppy-miR-410, ppy-miR-411, ppy-miR-412, ppy-miR-421, ppy-miR-422a, ppy-miR-423-3p, ppy-miR-423-5p, ppy-miR-424, ppy-miR-425, ppy-miR-429, ppy-miR-431, ppy-miR-432, ppy-miR-433, ppy-miR-4427, ppy-miR-4446, ppy-miR-4451, ppy-miR-448, ppy-miR-4484, ppy-miR-449a, ppy-miR-449b, ppy-miR-450a, ppy-miR-450b-3p, ppy-miR-450b-5p, ppy-miR-451, ppy-miR-4515, ppy-miR-452, ppy-miR-4520a, ppy-miR-4526, ppy-miR-4529, ppy-miR-453, ppy-miR-454, ppy-miR-455-3p, ppy-miR-455-5p, ppy-miR-4637, ppy-miR-4667, ppy-miR-4672, ppy-miR-4743, ppy-miR-4774, ppy-miR-4782, ppy-miR-4788, ppy-miR-4791, ppy-miR-4796, ppy-miR-4798, ppy-miR-484, ppy-miR-485-3p, ppy-miR-485-5p, ppy-miR-486-5p, ppy-miR-487a, ppy-miR-487b, ppy-miR-488, ppy-miR-489, ppy-miR-490-3p, ppy-miR-490-5p, ppy-miR-491-3p, ppy-miR-491-5p, ppy-miR-492, ppy-miR-493, ppy-miR-494, ppy-miR-495, ppy-miR-496, ppy-miR-497, ppy-miR-498, ppy-miR-499-3p, ppy-miR-499-5p, ppy-miR-500, ppy-miR-502-3p, ppy-miR-502-5p, ppy-miR-503, ppy-miR-504, ppy-miR-505, ppy-miR-506, ppy-miR-507, ppy-miR-508-3p, ppy-miR-508-5p, ppy-miR-509-3p, ppy-miR-509-5p, ppy-miR-510, ppy-miR-511, ppy-miR-512-3p, ppy-miR-513a-1-3p, ppy-miR-513a-2-3p, ppy-miR-513a-5p, ppy-miR-513b, ppy-miR-514, ppy-miR-515-3p, ppy-miR-515-5p, ppy-miR-516a, ppy-miR-516a-3p, ppy-miR-516a-5p, ppy-miR-516b, ppy-miR-517a, ppy-miR-517c-??, ppy-miR-517c-3p, ppy-miR-517c-5p, ppy-miR-518a-3p, ppy-miR-518a-5p, ppy-miR-518c, ppy-miR-518e-3p, ppy-miR-518e-5p, ppy-miR-518f, ppy-miR-519a, ppy-miR-519b, ppy-miR-519d, ppy-miR-519e, ppy-miR-520a-3p, ppy-miR-520a-5p, ppy-miR-520b, ppy-miR-520c-3p, ppy-miR-520c-5p, ppy-miR-520d-3p, ppy-miR-520d-5p, ppy-miR-520e, ppy-miR-520f, ppy-miR-520g, ppy-miR-520h, ppy-miR-520i, ppy-miR-521, ppy-miR-522, ppy-miR-523, ppy-miR-524-3p, ppy-miR-524-5p, ppy-miR-525-3p, ppy-miR-525-5p, ppy-miR-526a, ppy-miR-526b, ppy-miR-527, ppy-miR-532-3p, ppy-miR-532-5p, ppy-miR-539, ppy-miR-541, ppy-miR-542-3p, ppy-miR-542-5p, ppy-miR-543, ppy-miR-544, ppy-miR-545, ppy-miR-548a, ppy-miR-548c, ppy-miR-548e, ppy-miR-548f, ppy-miR-548h, ppy-miR-549, ppy-miR-551a, ppy-miR-551b, ppy-miR-553, ppy-miR-554, ppy-miR-555, ppy-miR-556-3p, ppy-miR-556-5p, ppy-miR-557, ppy-miR-558, ppy-miR-559, ppy-miR-562, ppy-miR-563, ppy-miR-564, ppy-miR-567, ppy-miR-568, ppy-miR-569, ppy-miR-571, ppy-miR-573, ppy-miR-575, ppy-miR-576-3p, ppy-miR-576-5p, ppy-miR-577, ppy-miR-578, ppy-miR-579, ppy-miR-580, ppy-miR-581, ppy-miR-582-3p, ppy-miR-582-5p, ppy-miR-583, ppy-miR-584, ppy-miR-586, ppy-miR-587, ppy-miR-588, ppy-miR-589, ppy-miR-590-3p, ppy-miR-590-5p, ppy-miR-591, ppy-miR-592, ppy-miR-593, ppy-miR-595, ppy-miR-596, ppy-miR-597, ppy-miR-598, ppy-miR-599, ppy-miR-600, ppy-miR-601, ppy-miR-602, ppy-miR-603, ppy-miR-604, ppy-miR-605, ppy-miR-606, ppy-miR-608, ppy-miR-609, ppy-miR-610, ppy-miR-611, ppy-miR-612, ppy-miR-613, ppy-miR-614, ppy-miR-615-3p, ppy-miR-615-5p, ppy-miR-616, ppy-miR-617, ppy-miR-618, ppy-miR-619, ppy-miR-621, ppy-miR-622, ppy-miR-623, ppy-miR-624, ppy-miR-626, ppy-miR-628-3p, ppy-miR-628-5p, ppy-miR-630, ppy-miR-631, ppy-miR-632, ppy-miR-633, ppy-miR-634, ppy-miR-636, ppy-miR-637, ppy-miR-638, ppy-miR-639, ppy-miR-640, ppy-miR-645, ppy-miR-646, ppy-miR-647, ppy-miR-648, ppy-miR-650, ppy-miR-651, ppy-miR-652, ppy-miR-6529, ppy-miR-653, ppy-miR-654-3p, ppy-miR-654-5p, ppy-miR-655, ppy-miR-656, ppy-miR-658, ppy-miR-660, ppy-miR-663b, ppy-miR-664, ppy-miR-668, ppy-miR-671-3p, ppy-miR-671-5p, ppy-miR-675a, ppy-miR-676, ppy-miR-7, ppy-miR-708, ppy-miR-744, ppy-miR-758, ppy-miR-760, ppy-miR-765, ppy-miR-766, ppy-miR-767-3p, ppy-miR-767-5p, ppy-miR-770-5p, ppy-miR-802, ppy-miR-873, ppy-miR-874, ppy-miR-875-3p, ppy-miR-875-5p, ppy-miR-876-3p, ppy-miR-876-5p, ppy-miR-877, ppy-miR-885-3p, ppy-miR-885-5p, ppy-miR-887, ppy-miR-888, ppy-miR-889, ppy-miR-890, ppy-miR-891a, ppy-miR-891b, ppy-miR-892a, ppy-miR-892b, ppy-miR-9, ppy-miR-92, ppy-miR-920, ppy-miR-921, ppy-miR-922, ppy-miR-924, ppy-miR-92b, ppy-miR-93, ppy-miR-933, ppy-miR-934, ppy-miR-936, ppy-miR-937, ppy-miR-938, ppy-miR-939, ppy-miR-942, ppy-miR-943, ppy-miR-944, ppy-miR-95, ppy-miR-96, ppy-miR-98, ppy-miR-99a, pra-miR8788a-3p, pra-miR8788a-5p, pra-miR8788b-3p, pra-miR8788b-5p, prd-let-7-3p, prd-let-7-5p, prd-lin-4-3p, prd-lin-4-5p, prd-miR-1-3p, prd-miR-1-5p, prd-miR-100-3p, prd-miR-100-5p, prd-miR-1175-3p, prd-miR-1175-5p, prd-miR-124-3p, prd-miR-124-5p, prd-miR-184-3p, prd-miR-184-5p, prd-miR-2-3p, prd-miR-2-5p, prd-miR-234-3p, prd-miR-234-5p, prd-miR-235-3p, prd-miR-235-5p, prd-miR-236-3p, prd-miR-236-5p, prd-miR-239-3p, prd-miR-239-5p, prd-miR-240-3p, prd-miR-240-5p, prd-miR-242-3p, prd-miR-242-5p, prd-miR-252-3p, prd-miR-252-5p, prd-miR-255-3p, prd-miR-255-5p, prd-miR-277-3p, prd-miR-277-5p, prd-miR-34-3p, prd-miR-34-5p, prd-miR-35a-3p, prd-miR-35a-5p, prd-miR-35b-3p, prd-miR-35b-5p, prd-miR-36-3p, prd-miR-36-5p, prd-miR-360-3p, prd-miR-360-5p, prd-miR-37-3p, prd-miR-37-5p, prd-miR-39-3p, prd-miR-39-5p, prd-miR-40-3p, prd-miR-40-5p, prd-miR-44-3p, prd-miR-44-5p, prd-miR-46-3p, prd-miR-46-5p, prd-miR-4816-3p, prd-miR-4816-5p, prd-miR-49-3p, prd-miR-49-5p, prd-miR-50-3p, prd-miR-50-5p, prd-miR-5358a-3p, prd-miR-5358a-5p, prd-miR-5358b-3p, prd-miR-5358b-5p, prd-miR-5359-3p, prd-miR-5359-5p, prd-miR-5360-3p, prd-miR-5360-5p, prd-miR-5594-3p, prd-miR-5594-5p, prd-miR-58-3p, prd-miR-58-5p, prd-miR-5960-3p, prd-miR-5960-5p, prd-miR-60-3p, prd-miR-60-5p, prd-miR-61-3p, prd-miR-61-5p, prd-miR-67-3p, prd-miR-67-5p, prd-miR-71-3p, prd-miR-71-5p, prd-miR-72-3p, prd-miR-72-5p, prd-miR-7579-3p, prd-miR-7579-5p, prd-miR-76-3p, prd-miR-76-5p, prd-miR-7865-3p, prd-miR-7865-5p, prd-miR-7866-3p, prd-miR-7866-5p, prd-miR-7867-3p, prd-miR-7867-5p, prd-miR-7868-3p, prd-miR-7868-5p, prd-miR-7869-3p, prd-miR-7869-5p, prd-miR-7870-3p, prd-miR-7870-5p, prd-miR-7871-3p, prd-miR-7871-5p, prd-miR-7872-3p, prd-miR-7872-5p, prd-miR-7873a-3p, prd-miR-7873a-5p, prd-miR-7873b-3p, prd-miR-7873b-5p, prd-miR-7874-3p, prd-miR-7874-5p, prd-miR-7875-3p, prd-miR-7875-5p, prd-miR-7876-3p, prd-miR-7876-5p, prd-miR-7877-3p, prd-miR-7877-5p, prd-miR-7878-3p, prd-miR-7878-5p, prd-miR-7879-3p, prd-miR-7879-5p, prd-miR-7880a-3p, prd-miR-7880a-5p, prd-miR-7880b-3p, prd-miR-7880b-5p, prd-miR-7880c-3p, prd-miR-7880c-5p, prd-miR-7881-3p, prd-miR-7881-5p, prd-miR-7882-3p, prd-miR-7882-5p, prd-miR-7883a-3p, prd-miR-7883a-5p, prd-miR-7883b-3p, prd-miR-7883b-5p, prd-miR-7883c-3p, prd-miR-7883c-5p, prd-miR-7884-3p, prd-miR-7884-5p, prd-miR-7885-3p, prd-miR-7885-5p, prd-miR-7886-3p, prd-miR-7886-5p, prd-miR-7887-3p, prd-miR-7887-5p, prd-miR-7888-3p, prd-miR-7888-5p, prd-miR-7889-3p, prd-miR-7889-5p, prd-miR-7890-3p, prd-miR-7890-5p, prd-miR-7891-3p, prd-miR-7891-5p, prd-miR-7892-3p, prd-miR-7892-5p, prd-miR-7893-3p, prd-miR-7893-5p, prd-miR-7894-3p, prd-miR-7894-5p, prd-miR-7895-3p, prd-miR-7895-5p, prd-miR-7896-3p, prd-miR-7896-5p, prd-miR-7897-3p, prd-miR-7897-5p, prd-miR-7899-3p, prd-miR-7899-5p, prd-miR-7900-3p, prd-miR-7900-5p, prd-miR-7901-3p, prd-miR-7901-5p, prd-miR-7902-3p, prd-miR-7902-5p, prd-miR-7903-3p, prd-miR-7903-5p, prd-miR-7904-3p, prd-miR-7904-5p, prd-miR-7905-3p, prd-miR-7905-5p, prd-miR-7906-3p, prd-miR-7906-5p, prd-miR-7907a-3p, prd-miR-7907a-5p, prd-miR-7907b-3p, prd-miR-7907b-5p, prd-miR-7908-3p, prd-miR-7908-5p, prd-miR-7909-3p, prd-miR-7909-5p, prd-miR-7910a-3p, prd-miR-7910a-5p, prd-miR-7910b-3p, prd-miR-7910b-5p, prd-miR-7911a-3p, prd-miR-7911a-5p, prd-miR-7911b-3p, prd-miR-7911b-5p, prd-miR-7911c-3p, prd-miR-7911c-5p, prd-miR-7912-3p, prd-miR-7912-5p, prd-miR-7913-3p, prd-miR-7913-5p, prd-miR-7914-3p, prd-miR-7914-5p, prd-miR-7915-3p, prd-miR-7915-5p, prd-miR-7916-3p, prd-miR-7916-5p, prd-miR-7917-3p, prd-miR-7917-5p, prd-miR-7918a-3p, prd-miR-7918a-5p, prd-miR-7918b-3p, prd-miR-7918b-5p, prd-miR-7918c-3p, prd-miR-7918c-5p, prd-miR-7918d-3p, prd-miR-7918d-5p, prd-miR-7918e-3p, prd-miR-7918e-5p, prd-miR-7918f-3p, prd-miR-7918f-5p, prd-miR-7919-3p, prd-miR-7919-5p, prd-miR-7920-3p, prd-miR-7920-5p, prd-miR-7921-3p, prd-miR-7921-5p, prd-miR-7922-3p, prd-miR-7922-5p, prd-miR-7923-3p, prd-miR-7923-5p, prd-miR-7924-3p, prd-miR-7924-5p, prd-miR-7925-3p, prd-miR-7925-5p, prd-miR-7926-3p, prd-miR-7926-5p, prd-miR-7927a-3p, prd-miR-7927a-5p, prd-miR-7927b-3p, prd-miR-7927b-5p, prd-miR-7927c-3p, prd-miR-7927c-5p, prd-miR-7927d-3p, prd-miR-7927d-5p, prd-miR-7927e-3p, prd-miR-7927e-5p, prd-miR-7928-3p, prd-miR-7928-5p, prd-miR-7929-3p, prd-miR-7929-5p, prd-miR-7930-3p, prd-miR-7930-5p, prd-miR-7931-3p, prd-miR-7931-5p, prd-miR-7932-3p, prd-miR-7932-5p, prd-miR-7933-3p, prd-miR-7933-5p, prd-miR-7934-3p, prd-miR-7934-5p, prd-miR-7935-3p, prd-miR-7935-5p, prd-miR-7936-3p, prd-miR-7936-5p, prd-miR-7937-3p, prd-miR-7937-5p, prd-miR-7938-3p, prd-miR-7938-5p, prd-miR-7939a-3p, prd-miR-7939a-5p, prd-miR-7939b-3p, prd-miR-7939b-5p, prd-miR-7939c-3p, prd-miR-7939c-5p, prd-miR-7939d-3p, prd-miR-7939d-5p, prd-miR-7939e-3p, prd-miR-7939e-5p, prd-miR-7939f-3p, prd-miR-7939f-5p, prd-miR-7939g-3p, prd-miR-7939g-5p, prd-miR-7939 h-3p, prd-miR-7939 h-5p, prd-miR-7939i-3p, prd-miR-7939i-5p, prd-miR-7939j-3p, prd-miR-7939j-5p, prd-miR-7940a-3p, prd-miR-7940a-5p, prd-miR-7940b-3p, prd-miR-7940b-5p, prd-miR-7940c-3p, prd-miR-7940c-5p, prd-miR-7941-3p, prd-miR-7941-5p, prd-miR-7942-3p, prd-miR-7942-5p, prd-miR-7943-3p, prd-miR-7943-5p, prd-miR-7944-3p, prd-miR-7944-5p, prd-miR-7945-3p, prd-miR-7945-5p, prd-miR-7946-3p, prd-miR-7946-5p, prd-miR-7947-3p, prd-miR-7947-5p, prd-miR-7948a-3p, prd-miR-7948a-5p, prd-miR-7948b-3p, prd-miR-7948b-5p, prd-miR-7948c-3p, prd-miR-7948c-5p, prd-miR-7948d-3p, prd-miR-7948d-5p, prd-miR-7949-3p, prd-miR-7949-5p, prd-miR-7950a-3p, prd-miR-7950a-5p, prd-miR-7950b-3p, prd-miR-7950b-5p, prd-miR-7950c-3p, prd-miR-7950c-5p, prd-miR-7951-3p, prd-miR-7951-5p, prd-miR-7952-3p, prd-miR-7952-5p, prd-miR-7953-3p, prd-miR-7953-5p, prd-miR-7954-3p, prd-miR-7954-5p, prd-miR-7955-3p, prd-miR-7955-5p, prd-miR-7956-3p, prd-miR-7956-5p, prd-miR-7957a-3p, prd-miR-7957a-5p, prd-miR-7957b-3p, prd-miR-7957b-5p, prd-miR-7957c-3p, prd-miR-7957c-5p, prd-miR-7957d-3p, prd-miR-7957d-5p, prd-miR-7958-3p, prd-miR-7958-5p, prd-miR-7959a-3p, prd-miR-7959a-5p, prd-miR-7959b-3p, prd-miR-7959b-5p, prd-miR-7960-3p, prd-miR-7960-5p, prd-miR-7961-3p, prd-miR-7961-5p, prd-miR-7962-3p, prd-miR-7962-5p, prd-miR-7963-3p, prd-miR-7963-5p, prd-miR-7964a-3p, prd-miR-7964a-5p, prd-miR-7964b-3p, prd-miR-7964b-5p, prd-miR-7965-3p, prd-miR-7965-5p, prd-miR-7966a-3p, prd-miR-7966a-5p, prd-miR-7966b-3p, prd-miR-7966b-5p, prd-miR-7967-3p, prd-miR-7967-5p, prd-miR-7968-3p, prd-miR-7968-5p, prd-miR-7969-3p, prd-miR-7969-5p, prd-miR-7970-3p, prd-miR-7970-5p, prd-miR-7971-3p, prd-miR-7971-5p, prd-miR-81-3p, prd-miR-81-5p, prd-miR-84-3p, prd-miR-84-5p, prd-miR-86-3p, prd-miR-86-5p, prd-miR-87-3p, prd-miR-87-5p, prd-miR-993-3p, prd-miR-993-5p, prd-miR-9a-3p, prd-miR-9a-5p, prd-miR-9b-3p, prd-miR-9b-5p, prv-miR-LLT1, prv-miR-LLT10a, prv-miR-LLT10b, prv-miR-LLTlla-3p, prv-miR-LLTlla-5p, prv-miR-LLT11b-3p, prv-miR-LLT11b-5p, prv-miR-LLT12, prv-miR-LLT2, prv-miR-LLT3, prv-miR-LLT4, prv-miR-LLT5, prv-miR-LLT6, prv-miR-LLT7-3p, prv-miR-LLT7-5p, prv-miR-LLT8-3p, prv-miR-LLT8-5p, prv-miR-LLT9, psj-miR8788a-3p, psj-miR8788a-5p, psj-miR8788b-3p, psj-miR8788b-5p, pta-miR1309, pta-miR1311, pta-miR1312, pta-miR1313, pta-miR1314, pta-miR1315, pta-miR1316, pta-miR156a, pta-miR156b, pta-miR159a, pta-miR159b, pta-miR159c, pta-miR166a, pta-miR166b, pta-miR166c, pta-miR171, pta-miR319, pta-miR390, pta-miR396, pta-miR398, pta-miR408, pta-miR482a, pta-miR482b, pta-miR482c, pta-miR482d, pta-miR946a-3p, pta-miR946a-5p, pta-miR946b, pta-miR947, pta-miR948, pta-miR949, pta-miR950a, pta-miR950b, pta-miR951, pta-miR952a, pta-miR952b, ptc-miR1444a, ptc-miR1444b, ptc-miR1444c, ptc-miR1444d, ptc-miR1444e, ptc-miR1445, ptc-miR1446a, ptc-miR1446b, ptc-miR1446c, ptc-miR1446d, ptc-miR1446e, ptc-miR1447, ptc-miR1448, ptc-miR1449, ptc-miR1450, ptc-miR156a, ptc-miR156b, ptc-miR156c, ptc-miR156d, ptc-miR156e, ptc-miR156f, ptc-miR156g, ptc-miR156h, ptc-miR156i, ptc-miR156j, ptc-miR156k, ptc-miR1561, ptc-miR159a, ptc-miR159b, ptc-miR159c, ptc-miR159d, ptc-miR159e, ptc-miR160a, ptc-miR160b-3p, ptc-miR160b-5p, ptc-miR160c-3p, ptc-miR160c-5p, ptc-miR160d, ptc-miR160e-3p, ptc-miR160e-5p, ptc-miR160f, ptc-miR160g, ptc-miR160h, ptc-miR162a, ptc-miR162b, ptc-miR164a, ptc-miR164b, ptc-miR164c, ptc-miR164d, ptc-miR164e, ptc-miR164f, ptc-miR166a, ptc-miR166b, ptc-miR166c, ptc-miR166d, ptc-miR166e, ptc-miR166f, ptc-miR166g, ptc-miR166h, ptc-miR166i, ptc-miR166j, ptc-miR166k, ptc-miR1661, ptc-miR166m, ptc-miR166n, ptc-miR1660, ptc-miR166p, ptc-miR166q, ptc-miR167a, ptc-miR167b, ptc-miR167c, ptc-miR167d, ptc-miR167e, ptc-miR167f-3p, ptc-miR167f-5p, ptc-miR167g-3p, ptc-miR167g-5p, ptc-miR167 h-3p, ptc-miR167 h-5p, ptc-miR168a-3p, ptc-miR168a-5p, ptc-miR168b-3p, ptc-miR168b-5p, ptc-miR169a, ptc-miR169aa, ptc-miR169ab, ptc-miR169ac, ptc-miR169ad, ptc-miR169ae, ptc-miR169af, ptc-miR169ag, ptc-miR169b-3p, ptc-miR169b-5p, ptc-miR169c, ptc-miR169d, ptc-miR169e, ptc-miR169f, ptc-miR169g, ptc-miR169h, ptc-miR169i, ptc-miR169j, ptc-miR169k, ptc-miR1691, ptc-miR169m, ptc-miR169n-3p, ptc-miR169n-5p, ptc-miR1690, ptc-miR169p, ptc-miR169q, ptc-miR169r, ptc-miR169s, ptc-miR169t, ptc-miR169u-3p, ptc-miR169u-5p, ptc-miR169v, ptc-miR169w, ptc-miR169x, ptc-miR169y, ptc-miR169z, ptc-miR171a-3p, ptc-miR171a-5p, ptc-miR171b, ptc-miR171c, ptc-miR171d, ptc-miR171e, ptc-miR171f, ptc-miR171g-3p, ptc-miR171g-5p, ptc-miR171 h-3p, ptc-miR171h-5p, ptc-miR171i, ptc-miR171j, ptc-miR171k, ptc-miR1711-3p, ptc-miR1711-5p, ptc-miR171m, ptc-miR172a, ptc-miR172b-3p, ptc-miR172b-5p, ptc-miR172c, ptc-miR172d, ptc-miR172e, ptc-miR172f, ptc-miR172g-3p, ptc-miR172g-5p, ptc-miR172 h-3p, ptc-miR172h-5p, ptc-miR172i, ptc-miR2111a, ptc-miR2111b, ptc-miR319a, ptc-miR319b, ptc-miR319c, ptc-miR319d, ptc-miR319e, ptc-miR319f, ptc-miR319g, ptc-miR319h, ptc-miR319i, ptc-miR3627a, ptc-miR3627b, ptc-miR390a, ptc-miR390b, ptc-miR390c, ptc-miR390d-3p, ptc-miR390d-5p, ptc-miR393a-3p, ptc-miR393a-5p, ptc-miR393b-3p, ptc-miR393b-5p, ptc-miR393c, ptc-miR394a-3p, ptc-miR394a-5p, ptc-miR394b-3p, ptc-miR394b-5p, ptc-miR395a, ptc-miR395b, ptc-miR395c, ptc-miR395d, ptc-miR395e, ptc-miR395f, ptc-miR395g, ptc-miR395h, ptc-miR395i, ptc-miR395j, ptc-miR395k, ptc-miR396a, ptc-miR396b, ptc-miR396c, ptc-miR396d, ptc-miR396e-3p, ptc-miR396e-5p, ptc-miR396f, ptc-miR396g-3p, ptc-miR396g-5p, ptc-miR397a, ptc-miR397b, ptc-miR397c, ptc-miR398a, ptc-miR398b, ptc-miR398c-3p, ptc-miR398c-5p, ptc-miR399a, ptc-miR399b, ptc-miR399c, ptc-miR399d, ptc-miR399e, ptc-miR399f, ptc-miR399g, ptc-miR399h, ptc-miR399i, ptc-miR399j, ptc-miR403a, ptc-miR403b, ptc-miR403c-3p, ptc-miR403c-5p, ptc-miR403d, ptc-miR408-3p, ptc-miR408-5p, ptc-miR472a, ptc-miR472b, ptc-miR474a, ptc-miR474b, ptc-miR474c, ptc-miR475a-3p, ptc-miR475a-5p, ptc-miR475b-3p, ptc-miR475b-5p, ptc-miR475c, ptc-miR475d-3p, ptc-miR475d-5p, ptc-miR476a, ptc-miR476b, ptc-miR477a-3p, ptc-miR477a-5p, ptc-miR477b, ptc-miR477c, ptc-miR477d-3p, ptc-miR477d-5p, ptc-miR477e-3p, ptc-miR477e-5p, ptc-miR477f, ptc-miR478a, ptc-miR478b, ptc-miR478c, ptc-miR478d, ptc-miR478e, ptc-miR478f, ptc-miR478h, ptc-miR478i, ptc-miR478j, ptc-miR478k, ptc-miR4781, ptc-miR478m, ptc-miR478n, ptc-miR4780, ptc-miR478p, ptc-miR478q, ptc-miR478r, ptc-miR478s, ptc-miR480, ptc-miR481a, ptc-miR481b, ptc-miR481c, ptc-miR481d, ptc-miR482a.1, ptc-miR482a.2, ptc-miR482b-3p, ptc-miR482b-5p, ptc-miR482c-3p, ptc-miR482c-5p, ptc-miR482d-3p, ptc-miR482d-5p, ptc-miR530a, ptc-miR530b, ptc-miR536, ptc-miR6421-3p, ptc-miR6421-5p, ptc-miR6422, ptc-miR6423, ptc-miR6425a-3p, ptc-miR6425a-5p, ptc-miR6425b-3p, ptc-miR6425b-5p, ptc-miR6425c-3p, ptc-miR6425c-5p, ptc-miR6425d-3p, ptc-miR6425d-5p, ptc-miR6425e, ptc-miR6426a, ptc-miR6426b, ptc-miR6427-3p, ptc-miR6427-5p, ptc-miR6428, ptc-miR6429, ptc-miR6430, ptc-miR6431, ptc-miR6432, ptc-miR6434, ptc-miR6435, ptc-miR6436, ptc-miR6437a, ptc-miR6437b, ptc-miR6438a, ptc-miR6438b, ptc-miR6439a, ptc-miR6439b, ptc-miR6440a, ptc-miR6440b, ptc-miR6440c, ptc-miR6440d, ptc-miR6441, ptc-miR6442, ptc-miR6443, ptc-miR6444, ptc-miR6445a, ptc-miR6445b, ptc-miR6446, ptc-miR6447, ptc-miR6448, ptc-miR6449, ptc-miR6450a, ptc-miR6450b, ptc-miR6451, ptc-miR6452, ptc-miR6454, ptc-miR6455, ptc-miR6456, ptc-miR6457a, ptc-miR6457b, ptc-miR6458, ptc-miR6459a-3p, ptc-miR6459a-5p, ptc-miR6459b, ptc-miR6460, ptc-miR6461, ptc-miR6462a, ptc-miR6462b, ptc-miR6462c-3p, ptc-miR6462c-5p, ptc-miR6462d, ptc-miR6462e, ptc-miR6462f, ptc-miR6463, ptc-miR6464, ptc-miR6465, ptc-miR6466-3p, ptc-miR6466-5p, ptc-miR6467, ptc-miR6468-3p, ptc-miR6468-5p, ptc-miR6469, ptc-miR6470, ptc-miR6471, ptc-miR6472, ptc-miR6473, ptc-miR6474, ptc-miR6475, ptc-miR6476a, ptc-miR6476b, ptc-miR6476c, ptc-miR6477, ptc-miR6478, ptc-miR6479, ptc-miR6480, ptc-miR7812, ptc-miR7813, ptc-miR7814, ptc-miR7815, ptc-miR7816, ptc-miR7817a, ptc-miR7817b, ptc-miR7818, ptc-miR7819, ptc-miR7820, ptc-miR7821, ptc-miR7822, ptc-miR7823, ptc-miR7824, ptc-miR7825, ptc-miR7826, ptc-miR7827, ptc-miR7828, ptc-miR7829, ptc-miR7830, ptc-miR7831, ptc-miR7832, ptc-miR7833, ptc-miR7834, ptc-miR7835, ptc-miR7836, ptc-miR7837, ptc-miR7838, ptc-miR7839, ptc-miR7840, ptc-miR7841, ptc-miR7842, ptc-miR827, ptc-miR828a, ptc-miR828b-3p, ptc-miR828b-5p, pte-bantam-3p, pte-bantam-5p, pte-iab-4-2-3p, pte-iab-4-3p, pte-iab-4-5p, pte-iab-8-3p, pte-iab-8-5p, pte-let-7-3p, pte-let-7-5p, pte-miR-1-3p, pte-miR-1-5p, pte-miR-10-3p, pte-miR-10-5p, pte-miR-100a-3p, pte-miR-100a-5p, pte-miR-100b-3p, pte-miR-100b-5p, pte-miR-11938-3p, pte-miR-11938-5p, pte-miR-11939-3p, pte-miR-11939-5p, pte-miR-11940-3p, pte-miR-11940-5p, pte-miR-11941-3p, pte-miR-11941-5p, pte-miR-11942a-3p, pte-miR-11942a-5p, pte-miR-11942b-3p, pte-miR-11942b-5p, pte-miR-11942c-3p, pte-miR-11942c-5p, pte-miR-11942d-3p, pte-miR-11942d-5p, pte-miR-11943-3p, pte-miR-11943-5p, pte-miR-11944-3p, pte-miR-11944-5p, pte-miR-11945-3p, pte-miR-11945-5p, pte-miR-11946-3p, pte-miR-11946-5p, pte-miR-11947a-3p, pte-miR-11947a-5p, pte-miR-11947b-3p, pte-miR-11947b-5p, pte-miR-11948-3p, pte-miR-11948-5p, pte-miR-11949-3p, pte-miR-11949-5p, pte-miR-11950-3p, pte-miR-11950-5p, pte-miR-11951a-3p, pte-miR-11951a-5p, pte-miR-11951b-3p, pte-miR-11951b-5p, pte-miR-11952-3p, pte-miR-11952-5p, pte-miR-11953-3p, pte-miR-11953-5p, pte-miR-11954-3p, pte-miR-11954-5p, pte-miR-11955-3p, pte-miR-11955-5p, pte-miR-11956-3p, pte-miR-11956-5p, pte-miR-11957-3p, pte-miR-11957-5p, pte-miR-11958-3p, pte-miR-11958-5p, pte-miR-11959-3p, pte-miR-11959-5p, pte-miR-11960-3p, pte-miR-11960-5p, pte-miR-11961a-3p, pte-miR-11961a-5p, pte-miR-11961b-3p, pte-miR-11961b-5p, pte-miR-11961c-3p, pte-miR-11961c-5p, pte-miR-11962a-3p, pte-miR-11962a-5p, pte-miR-11962b-3p, pte-miR-11962b-5p, pte-miR-11963a-3p, pte-miR-11963a-5p, pte-miR-11963b-3p, pte-miR-11963b-5p, pte-miR-11964-3p, pte-miR-11964-5p, pte-miR-11965-2-5p, pte-miR-11965-3-5p, pte-miR-11965-3p, pte-miR-11965-5p, pte-miR-11966-3p, pte-miR-11966-5p, pte-miR-11967-3p, pte-miR-11967-5p, pte-miR-11968-3p, pte-miR-11968-5p, pte-miR-125a-3p, pte-miR-125a-5p, pte-miR-125b-3p, pte-miR-125b-5p, pte-miR-14-3p, pte-miR-14-5p, pte-miR-184-2-5p, pte-miR-184-3p, pte-miR-184-5p, pte-miR-190-3p, pte-miR-190-5p, pte-miR-193a-3p, pte-miR-193a-5p, pte-miR-193b-3p, pte-miR-193b-5p, pte-miR-210-3p, pte-miR-210-5p, pte-miR-263a-3p, pte-miR-263a-5p, pte-miR-263b-3p, pte-miR-263b-5p, pte-miR-275-3p, pte-miR-275-5p, pte-miR-276-2-5p, pte-miR-276-3p, pte-miR-276-5p, pte-miR-277a-3p, pte-miR-277a-5p, pte-miR-277b-3p, pte-miR-277b-5p, pte-miR-278a-3p, pte-miR-278a-5p, pte-miR-278b-3p, pte-miR-278b-5p, pte-miR-279-3p, pte-miR-279-5p, pte-miR-281-3p, pte-miR-281-5p, pte-miR-29a-3p, pte-miR-29a-5p, pte-miR-29b-3p, pte-miR-29b-5p, pte-miR-2a-3p, pte-miR-2a-5p, pte-miR-2b-2-5p, pte-miR-2b-3p, pte-miR-2b-5p, pte-miR-2c-3p, pte-miR-2c-5p, pte-miR-2d-3p, pte-miR-2d-5p, pte-miR-2e-3p, pte-miR-2e-5p, pte-miR-2f-3p, pte-miR-2f-5p, pte-miR-2g-2-5p, pte-miR-2g-3p, pte-miR-2g-5p, pte-miR-305-3p, pte-miR-305-5p, pte-miR-315-3p, pte-miR-315-5p, pte-miR-317-3p, pte-miR-317-5p, pte-miR-34-3p, pte-miR-34-5p, pte-miR-3477-3p, pte-miR-3477-5p, pte-miR-35a-3p, pte-miR-35a-5p, pte-miR-35b-3p, pte-miR-35b-5p, pte-miR-35c-3p, pte-miR-35c-5p, pte-miR-35d-3p, pte-miR-35d-5p, pte-miR-35e-3p, pte-miR-35e-5p, pte-miR-35f-3p, pte-miR-35f-5p, pte-miR-35g-3p, pte-miR-35g-5p, pte-miR-35 h-3p, pte-miR-35 h-5p, pte-miR-35i-3p, pte-miR-35i-5p, pte-miR-35j-3p, pte-miR-35j-5p, pte-miR-35k-3p, pte-miR-35k-5p, pte-miR-351-3p, pte-miR-351-5p, pte-miR-35m-3p, pte-miR-35m-5p, pte-miR-35n-3p, pte-miR-35n-5p, pte-miR-350-2-5p, pte-miR-350-3p, pte-miR-350-5p, pte-miR-35p-3p, pte-miR-35p-5p, pte-miR-35q-1-5p, pte-miR-35q-3p, pte-miR-35q-5p, pte-miR-35r-3p, pte-miR-35r-5p, pte-miR-35s-3p, pte-miR-35s-5p, pte-miR-35t-3p, pte-miR-35t-5p, pte-miR-35u-3p, pte-miR-35u-5p, pte-miR-35v-3p, pte-miR-35v-5p, pte-miR-35w-3p, pte-miR-35w-5p, pte-miR-35x-3p, pte-miR-35x-5p, pte-miR-35y-3p, pte-miR-35y-5p, pte-miR-35z-3p, pte-miR-35z-5p, pte-miR-375-2-5p, pte-miR-375-3p, pte-miR-375-5p, pte-miR-3931-3p, pte-miR-3931-5p, pte-miR-7-3p, pte-miR-7-5p, pte-miR-71-3p, pte-miR-71-5p, pte-miR-745-3p, pte-miR-745-5p, pte-miR-8-3p, pte-miR-8-5p, pte-miR-87a-3p, pte-miR-87a-5p, pte-miR-87b-3p, pte-miR-87b-5p, pte-miR-87c-3p, pte-miR-87c-5p, pte-miR-9-3p, pte-miR-9-5p, pte-miR-92a-3p, pte-miR-92a-5p, pte-miR-92b-3p, pte-miR-92b-5p, pte-miR-92c-3p, pte-miR-92c-5p, pte-miR-96-3p, pte-miR-96-5p, pte-miR-981-3p, pte-miR-981-5p, pte-miR-993a-3p, pte-miR-993a-5p, pte-miR-993b-2-3p, pte-miR-993b-3p, pte-miR-993b-5p, pti-miR5471, pti-miR5472, pti-miR5473, pti-miR5474, pti-miR5475, pti-miR5476, pti-miR5477, pti-miR5478, pti-miR5479, pti-miR5480, pti-miR5481, pti-miR5482, pti-miR5483, ptr-let-7a, ptr-let-7b, ptr-let-7c, ptr-let-7d, ptr-let-7e, ptr-let-7f, ptr-let-7g, ptr-let-7i, ptr-miR-1, ptr-miR-100, ptr-miR-101, ptr-miR-103, ptr-miR-105, ptr-miR-106a, ptr-miR-106b, ptr-miR-107, ptr-miR-10a, ptr-miR-10b, ptr-miR-1178, ptr-miR-1179, ptr-miR-1181, ptr-miR-1182, ptr-miR-1183, ptr-miR-1184, ptr-miR-1185, ptr-miR-1197, ptr-miR-1199-3p, ptr-miR-1199-5p, ptr-miR-1202, ptr-miR-1203, ptr-miR-1204, ptr-miR-1205, ptr-miR-1206, ptr-miR-1207, ptr-miR-1208, ptr-miR-122, ptr-miR-1224-3p, ptr-miR-1224-5p, ptr-miR-1225, ptr-miR-1226, ptr-miR-1227, ptr-miR-1233, ptr-miR-1234, ptr-miR-1236, ptr-miR-1237, ptr-miR-1244, ptr-miR-1245, ptr-miR-1246, ptr-miR-1247, ptr-miR-1248, ptr-miR-1249, ptr-miR-124a, ptr-miR-1250, ptr-miR-1251, ptr-miR-1252, ptr-miR-1253, ptr-miR-1255b, ptr-miR-1256, ptr-miR-1258, ptr-miR-125a, ptr-miR-125b, ptr-miR-126, ptr-miR-1260b, ptr-miR-1262, ptr-miR-1263, ptr-miR-1264, ptr-miR-1265, ptr-miR-1266, ptr-miR-1267, ptr-miR-127, ptr-miR-1271, ptr-miR-1272, ptr-miR-1275, ptr-miR-1276, ptr-miR-1278, ptr-miR-128, ptr-miR-1281, ptr-miR-1282, ptr-miR-1283a, ptr-miR-1283b, ptr-miR-1284, ptr-miR-1285, ptr-miR-1286, ptr-miR-1288, ptr-miR-1289, ptr-miR-129, ptr-miR-1290, ptr-miR-1291, ptr-miR-1292, ptr-miR-1293, ptr-miR-1294, ptr-miR-1295, ptr-miR-1296, ptr-miR-1297, ptr-miR-1298, ptr-miR-1299, ptr-miR-1302, ptr-miR-1303, ptr-miR-1306, ptr-miR-1307, ptr-miR-130a, ptr-miR-130b, ptr-miR-1322, ptr-miR-1323, ptr-miR-1324, ptr-miR-133a, ptr-miR-133b, ptr-miR-134, ptr-miR-135a, ptr-miR-135b, ptr-miR-136, ptr-miR-137, ptr-miR-138, ptr-miR-139, ptr-miR-140, ptr-miR-141, ptr-miR-142, ptr-miR-143, ptr-miR-144, ptr-miR-145, ptr-miR-146a, ptr-miR-146b, ptr-miR-147a, ptr-miR-147b, ptr-miR-148a, ptr-miR-148b, ptr-miR-149, ptr-miR-150, ptr-miR-151, ptr-miR-152, ptr-miR-153, ptr-miR-154, ptr-miR-155, ptr-miR-15a, ptr-miR-15b, ptr-miR-16, ptr-miR-17-3p, ptr-miR-17-5p, ptr-miR-181a-3p, ptr-miR-181a-5p, ptr-miR-181b, ptr-miR-181c, ptr-miR-181d, ptr-miR-182, ptr-miR-1825, ptr-miR-1827, ptr-miR-183, ptr-miR-184, ptr-miR-185, ptr-miR-186, ptr-miR-188, ptr-miR-18a, ptr-miR-18b, ptr-miR-190a, ptr-miR-190b, ptr-miR-191, ptr-miR-192, ptr-miR-193a, ptr-miR-193b, ptr-miR-194, ptr-miR-195, ptr-miR-196a, ptr-miR-196b, ptr-miR-197, ptr-miR-198, ptr-miR-199a-3p, ptr-miR-199a-5p, ptr-miR-199b, ptr-miR-19a, ptr-miR-19b, ptr-miR-200a, ptr-miR-200c, ptr-miR-202, ptr-miR-203, ptr-miR-204, ptr-miR-205, ptr-miR-206, ptr-miR-208a, ptr-miR-208b, ptr-miR-20a, ptr-miR-20b, ptr-miR-21, ptr-miR-210, ptr-miR-211, ptr-miR-2114, ptr-miR-214, ptr-miR-215, ptr-miR-216a, ptr-miR-216b, ptr-miR-217, ptr-miR-218, ptr-miR-219-1-3p, ptr-miR-219-2-3p, ptr-miR-219-5p, ptr-miR-22, ptr-miR-221, ptr-miR-222, ptr-miR-223, ptr-miR-224, ptr-miR-2355, ptr-miR-2392, ptr-miR-23a, ptr-miR-23b, ptr-miR-23c, ptr-miR-24, ptr-miR-25, ptr-miR-26a, ptr-miR-26b, ptr-miR-27a, ptr-miR-27b, ptr-miR-28, ptr-miR-296, ptr-miR-297, ptr-miR-298, ptr-miR-299, ptr-miR-29a, ptr-miR-29b, ptr-miR-29c, ptr-miR-300, ptr-miR-301a, ptr-miR-301b, ptr-miR-302a, ptr-miR-302b, ptr-miR-302c, ptr-miR-302d, ptr-miR-302e, ptr-miR-302f, ptr-miR-30a-3p, ptr-miR-30a-5p, ptr-miR-30b, ptr-miR-30c, ptr-miR-30d, ptr-miR-30e, ptr-miR-31, ptr-miR-3117, ptr-miR-3121, ptr-miR-3127, ptr-miR-3129, ptr-miR-3132, ptr-miR-3136, ptr-miR-3138, ptr-miR-3149, ptr-miR-3151, ptr-miR-3154, ptr-miR-3165, ptr-miR-3173, ptr-miR-3192, ptr-miR-3193, ptr-miR-32, ptr-miR-3200, ptr-miR-320a, ptr-miR-320b, ptr-miR-320c, ptr-miR-320d, ptr-miR-320e, ptr-miR-323, ptr-miR-324, ptr-miR-326, ptr-miR-328, ptr-miR-329, ptr-miR-330, ptr-miR-331, ptr-miR-335, ptr-miR-337, ptr-miR-338, ptr-miR-339, ptr-miR-33a, ptr-miR-33b, ptr-miR-340, ptr-miR-342, ptr-miR-345, ptr-miR-346, ptr-miR-34a, ptr-miR-34b, ptr-miR-34c, ptr-miR-361, ptr-miR-362, ptr-miR-365, ptr-miR-3660, ptr-miR-3664, ptr-miR-367, ptr-miR-369, ptr-miR-370, ptr-miR-371, ptr-miR-372, ptr-miR-373, ptr-miR-374a, ptr-miR-374b, ptr-miR-375, ptr-miR-376a, ptr-miR-376b, ptr-miR-376c, ptr-miR-377, ptr-miR-378a, ptr-miR-378b, ptr-miR-378e, ptr-miR-378j, ptr-miR-379, ptr-miR-380, ptr-miR-381, ptr-miR-382, ptr-miR-383, ptr-miR-3937, ptr-miR-3943, ptr-miR-409, ptr-miR-410, ptr-miR-411, ptr-miR-412, ptr-miR-421, ptr-miR-422a, ptr-miR-423, ptr-miR-424, ptr-miR-425, ptr-miR-431, ptr-miR-432, ptr-miR-433, ptr-miR-4423, ptr-miR-4428, ptr-miR-4446, ptr-miR-448, ptr-miR-449a, ptr-miR-449b, ptr-miR-449c, ptr-miR-4504, ptr-miR-450a, ptr-miR-450b, ptr-miR-451, ptr-miR-4510, ptr-miR-452, ptr-miR-4524a, ptr-miR-453, ptr-miR-4536, ptr-miR-454, ptr-miR-455, ptr-miR-4654, ptr-miR-4660, ptr-miR-4667, ptr-miR-4677, ptr-miR-4742, ptr-miR-4743, ptr-miR-484, ptr-miR-485, ptr-miR-486, ptr-miR-487a, ptr-miR-487b, ptr-miR-488, ptr-miR-489, ptr-miR-490, ptr-miR-491, ptr-miR-492, ptr-miR-494, ptr-miR-495, ptr-miR-496, ptr-miR-497, ptr-miR-498, ptr-miR-499, ptr-miR-500, ptr-miR-501, ptr-miR-502, ptr-miR-503, ptr-miR-504, ptr-miR-505, ptr-miR-506, ptr-miR-507, ptr-miR-508, ptr-miR-509a, ptr-miR-509b, ptr-miR-510, ptr-miR-511, ptr-miR-512, ptr-miR-513a, ptr-miR-513b, ptr-miR-514, ptr-miR-515, ptr-miR-516a, ptr-miR-516b, ptr-miR-517a, ptr-miR-517b, ptr-miR-518a, ptr-miR-518b, ptr-miR-518c, ptr-miR-518d, ptr-miR-518e, ptr-miR-518f, ptr-miR-518g-3p, ptr-miR-518g-5p, ptr-miR-519a, ptr-miR-519b, ptr-miR-519c, ptr-miR-519d, ptr-miR-519e, ptr-miR-519f-3p, ptr-miR-519f-5p, ptr-miR-520a, ptr-miR-520b, ptr-miR-520c, ptr-miR-520d, ptr-miR-520e, ptr-miR-520f, ptr-miR-520g, ptr-miR-520h, ptr-miR-521, ptr-miR-522, ptr-miR-523, ptr-miR-524, ptr-miR-525, ptr-miR-526a, ptr-miR-526b, ptr-miR-527, ptr-miR-532, ptr-miR-539, ptr-miR-543, ptr-miR-544, ptr-miR-545, ptr-miR-548a, ptr-miR-548b, ptr-miR-548c, ptr-miR-548e, ptr-miR-548f, ptr-miR-548h, ptr-miR-548i, ptr-miR-548j, ptr-miR-548k, ptr-miR-5481, ptr-miR-548n, ptr-miR-5480, ptr-miR-548p, ptr-miR-549, ptr-miR-550, ptr-miR-551a, ptr-miR-551b, ptr-miR-552, ptr-miR-553, ptr-miR-554, ptr-miR-555, ptr-miR-556, ptr-miR-557, ptr-miR-558, ptr-miR-559, ptr-miR-561, ptr-miR-562, ptr-miR-564, ptr-miR-567, ptr-miR-568, ptr-miR-569, ptr-miR-570, ptr-miR-571, ptr-miR-572, ptr-miR-575, ptr-miR-576, ptr-miR-577, ptr-miR-579, ptr-miR-580, ptr-miR-581, ptr-miR-582, ptr-miR-583, ptr-miR-584, ptr-miR-586, ptr-miR-587, ptr-miR-588, ptr-miR-589, ptr-miR-590, ptr-miR-591, ptr-miR-592, ptr-miR-593, ptr-miR-595, ptr-miR-596, ptr-miR-597, ptr-miR-598, ptr-miR-599, ptr-miR-600, ptr-miR-601, ptr-miR-602, ptr-miR-604, ptr-miR-605, ptr-miR-606, ptr-miR-609, ptr-miR-610, ptr-miR-612, ptr-miR-6127, ptr-miR-6128, ptr-miR-6129, ptr-miR-613, ptr-miR-6130, ptr-miR-6131, ptr-miR-6132, ptr-miR-6133, ptr-miR-6134, ptr-miR-614, ptr-miR-615, ptr-miR-616, ptr-miR-617, ptr-miR-618, ptr-miR-619, ptr-miR-621, ptr-miR-622, ptr-miR-623, ptr-miR-624, ptr-miR-626, ptr-miR-627, ptr-miR-628, ptr-miR-630, ptr-miR-631, ptr-miR-632, ptr-miR-633, ptr-miR-634, ptr-miR-635, ptr-miR-637, ptr-miR-640, ptr-miR-641, ptr-miR-642, ptr-miR-643, ptr-miR-645, ptr-miR-646, ptr-miR-649, ptr-miR-650, ptr-miR-651, ptr-miR-652, ptr-miR-653, ptr-miR-654, ptr-miR-655, ptr-miR-656, ptr-miR-657, ptr-miR-658, ptr-miR-660, ptr-miR-661, ptr-miR-663a, ptr-miR-663b, ptr-miR-664a, ptr-miR-664b, ptr-miR-665, ptr-miR-668, ptr-miR-670, ptr-miR-671, ptr-miR-676, ptr-miR-7, ptr-miR-708, ptr-miR-711, ptr-miR-718, ptr-miR-744, ptr-miR-758, ptr-miR-759, ptr-miR-760, ptr-miR-761, ptr-miR-762, ptr-miR-764, ptr-miR-765, ptr-miR-766, ptr-miR-767, ptr-miR-770, ptr-miR-802, ptr-miR-873, ptr-miR-874, ptr-miR-875, ptr-miR-876, ptr-miR-877, ptr-miR-885, ptr-miR-887, ptr-miR-889, ptr-miR-890, ptr-miR-891a, ptr-miR-891b, ptr-miR-892a, ptr-miR-892b, ptr-miR-9, ptr-miR-92, ptr-miR-920, ptr-miR-922, ptr-miR-924, ptr-miR-92b, ptr-miR-93, ptr-miR-933, ptr-miR-934, ptr-miR-935, ptr-miR-936, ptr-miR-937, ptr-miR-938, ptr-miR-939, ptr-miR-940, ptr-miR-942, ptr-miR-943, ptr-miR-944, ptr-miR-95, ptr-miR-96, ptr-miR-98, ptr-miR-99a, ptr-miR-99b, ptvpv2a-miR-P1-3p, ptvpv2a-miR-P1-5p, pvu-miR1514a, pvu-miR159a.1, pvu-miR159a.2, pvu-miR166a, pvu-miR2118, pvu-miR2119, pvu-miR319c, pvu-miR399a, pvu-miR482-3p, pvu-miR482-5p, pxy-bantam, pxy-miR-10-3p, pxy-miR-10-5p, pxy-miR-184, pxy-miR-193, pxy-miR-252, pxy-miR-2525-3p, pxy-miR-2525-5p, pxy-miR-263, pxy-miR-2733a, pxy-miR-2733b-3p, pxy-miR-2733b-5p, pxy-miR-274, pxy-miR-2756, pxy-miR-2767, pxy-miR-277, pxy-miR-279a, pxy-miR-279b-3p, pxy-miR-279b-5p, pxy-miR-281, pxy-miR-283, pxy-miR-2a-3p, pxy-miR-2a-5p, pxy-miR-2b, pxy-miR-306, pxy-miR-308, pxy-miR-3286, pxy-miR-6307, pxy-miR-6497, pxy-miR-745, pxy-miR-750, pxy-miR-7a, pxy-miR-7b, pxy-miR-8486, pxy-miR-8487, pxy-miR-8488, pxy-miR-8489a-3p, pxy-miR-8489a-5p, pxy-miR-8489b, pxy-miR-8490, pxy-miR-8491, pxy-miR-8492, pxy-miR-8493, pxy-miR-8494-3p, pxy-miR-8494-5p, pxy-miR-8495-3p, pxy-miR-8495-5p, pxy-miR-8496, pxy-miR-8497, pxy-miR-8498, pxy-miR-8499a, pxy-miR-8499b, pxy-miR-8499c, pxy-miR-8500, pxy-miR-8501, pxy-miR-8502, pxy-miR-8503, pxy-miR-8504, pxy-miR-8505, pxy-miR-8506, pxy-miR-8507-3p, pxy-miR-8507-5p, pxy-miR-8508, pxy-miR-8509, pxy-miR-8510a-3p, pxy-miR-8510a-5p, pxy-miR-8510a-8-3p, pxy-miR-8510b-3p, pxy-miR-8510b-5p, pxy-miR-8511, pxy-miR-8512, pxy-miR-8513-3p, pxy-miR-8513-5p, pxy-miR-8514-3p, pxy-miR-8514-5p, pxy-miR-8515, pxy-miR-8516, pxy-miR-8517a, pxy-miR-8517b, pxy-miR-8518, pxy-miR-8519, pxy-miR-8520-3p, pxy-miR-8520-5p, pxy-miR-8521a, pxy-miR-8521b, pxy-miR-8522, pxy-miR-8523, pxy-miR-8524-3p, pxy-miR-8524-5p, pxy-miR-8525, pxy-miR-8526, pxy-miR-8527, pxy-miR-8528a, pxy-miR-8528b, pxy-miR-8529, pxy-miR-8530-3p, pxy-miR-8530-5p, pxy-miR-8531, pxy-miR-8532-3p, pxy-miR-8532-5p, pxy-miR-8533, pxy-miR-8534-3p, pxy-miR-8534-5p, pxy-miR-8535-3p, pxy-miR-8535-5p, pxy-miR-8536a, pxy-miR-8536b-3p, pxy-miR-8536b-5p, pxy-miR-8537-3p, pxy-miR-8537-5p, pxy-miR-8538, pxy-miR-8539-3p, pxy-miR-8539-5p, pxy-miR-8540, pxy-miR-8541, pxy-miR-8542, pxy-miR-8543, pxy-miR-8544-3p, pxy-miR-8544-5p, pxy-miR-8545, pxy-miR-8546, pxy-miR-8547, pxy-miR-929, pxy-miR-965, pxy-miR-9a, pxy-miR-9b-3p, pxy-miR-9b-5p, racpv-miR-P1-3p, racpv-miR-P1-5p, rco-miR156a, rco-miR156b, rco-miR156c, rco-miR156d, rco-miR156e, rco-miR156f, rco-miR156g, rco-miR156h, rco-miR159, rco-miR160a, rco-miR160b, rco-miR160c, rco-miR162, rco-miR164a, rco-miR164b, rco-miR164c, rco-miR164d, rco-miR166a, rco-miR166b, rco-miR166c, rco-miR166d, rco-miR166e, rco-miR167a, rco-miR167b, rco-miR167c, rco-miR168, rco-miR169a, rco-miR169b, rco-miR169c, rco-miR171a, rco-miR171b, rco-miR171c, rco-miR171d, rco-miR171e, rco-miR171f, rco-miR171g, rco-miR172, rco-miR319a, rco-miR319b, rco-miR319c, rco-miR319d, rco-miR390a, rco-miR390b, rco-miR393, rco-miR395a, rco-miR395b, rco-miR395c, rco-miR395d, rco-miR395e, rco-miR396, rco-miR397, rco-miR398a, rco-miR398b, rco-miR399a, rco-miR399b, rco-miR399c, rco-miR399d, rco-miR399e, rco-miR399f, rco-miR403a, rco-miR403b, rco-miR408, rco-miR535, rgl-miR164, rgl-miR5137, rgl-miR5138, rgl-miR5139, rgl-miR5140, rgl-miR5141, rgl-miR5142, rgl-miR5573, rgl-miR5574, rgl-miR5575, rgl-miR5576, rgl-miR5577, rgl-miR5578, rgl-miR7797a, rgl-miR7797b, rgl-miR7798, rgl-miR7799, rgl-miR7800, rgl-miR7801, rgl-miR7802, rgl-miR7803a, rgl-miR7803b-3p, rgl-miR7803b-5p, rgl-miR7804-3p, rgl-miR7804-5p, rgl-miR7805-3p, rgl-miR7805-5p, rgl-miR7806, rgl-miR7807a-3p, rgl-miR7807a-5p, rgl-miR7807b-3p, rgl-miR7807b-5p, rgl-miR7808, rgl-miR7809, rgl-miR7810, rgl-miR7811, rgl-miR7972, rlcv-miR-rL1-1-3p, rlcv-miR-rL1-1-5p, rlcv-miR-rL1-10-3p, rlcv-miR-rL1-10-5p, rlcv-miR-rL1-11-3p, rlcv-miR-rL1-11-5p, rlcv-miR-rL1-12-3p, rlcv-miR-rL1-12-5p, rlcv-miR-rL1-13-3p, rlcv-miR-rL1-13-5p, rlcv-miR-rL1-14-3p, rlcv-miR-rL1-14-5p, rlcv-miR-rL1-15-3p, rlcv-miR-rL1-15-5p, rlcv-miR-rL1-16-3p, rlcv-miR-rL1-16-5p, rlcv-miR-rL1-17-3p, rlcv-miR-rL1-17-5p, rlcv-miR-rL1-18-3p, rlcv-miR-rL1-18-5p, rlcv-miR-rL1-19-3p, rlcv-miR-rL1-19-5p, rlcv-miR-rL1-2-3p, rlcv-miR-rL1-2-5p, rlcv-miR-rL1-20-3p, rlcv-miR-rL1-20-5p, rlcv-miR-rL1-21-3p, rlcv-miR-rL1-21-5p, rlcv-miR-rL1-22-3p, rlcv-miR-rL1-22-5p, rlcv-miR-rL1-23-3p, rlcv-miR-rL1-23-5p, rlcv-miR-rL1-24-3p, rlcv-miR-rL1-24-5p, rlcv-miR-rL1-25-3p, rlcv-miR-rL1-25-5p, rlcv-miR-rL1-26-3p, rlcv-miR-rL1-26-5p, rlcv-miR-rL1-27-3p, rlcv-miR-rL1-27-5p, rlcv-miR-rL1-28-3p, rlcv-miR-rL1-28-5p, rlcv-miR-rL1-29-3p, rlcv-miR-rL1-29-5p, rlcv-miR-rL1-3-3p, rlcv-miR-rL1-3-5p, rlcv-miR-rL1-30-3p, rlcv-miR-rL1-30-5p, rlcv-miR-rL1-31-3p, rlcv-miR-rL1-31-5p, rlcv-miR-rL1-32-3p, rlcv-miR-rL1-32-5p, rlcv-miR-rL1-33-3p, rlcv-miR-rL1-33-5p, rlcv-miR-rL1-34-3p, rlcv-miR-rL1-34-5p, rlcv-miR-rL1-35-3p, rlcv-miR-rL1-35-5p, rlcv-miR-rL1-4-3p, rlcv-miR-rL1-4-5p, rlcv-miR-rL1-5-3p, rlcv-miR-rL1-5-5p, rlcv-miR-rL1-6-3p, rlcv-miR-rL1-6-5p, rlcv-miR-rL1-7-3p, rlcv-miR-rL1-7-5p, rlcv-miR-rL1-8-3p, rlcv-miR-rL1-8-5p, rlcv-miR-rL1-9-3p, rlcv-miR-rL1-9-5p, rmi-miR-5316a, rmi-miR-5316b, rmi-miR-5317a, rmi-miR-5317b, rmi-miR-5318, rmi-miR-5319, rmi-miR-5320, rmi-miR-5321, rmi-miR-5322, rmi-miR-5323, rmi-miR-5324, rmi-miR-5325, rmi-miR-5326, rmi-miR-5327, rmi-miR-5328, rmi-miR-5329, rmi-miR-5330, rmi-miR-5331, rmi-miR-5332, rmi-miR-5333, rmi-miR-5334, rmi-miR-5335, rmi-miR-5336, rmi-miR-5341, rno-let-7a-1-3p, rno-let-7a-2-3p, rno-let-7a-5p, rno-let-7b-3p, rno-let-7b-5p, rno-let-7c-1-3p, rno-let-7c-2-3p, rno-let-7c-5p, rno-let-7d-3p, rno-let-7d-5p, rno-let-7e-3p, rno-let-7e-5p, rno-let-7f-1-3p, rno-let-7f-2-3p, rno-let-7f-5p, rno-let-7g-3p, rno-let-7g-5p, rno-let-7i-3p, rno-let-7i-5p, rno-miR-1-3p, rno-miR-1-5p, rno-miR-100-3p, rno-miR-100-5p, rno-miR-101a-3p, rno-miR-101a-5p, rno-miR-101b-3p, rno-miR-101b-5p, rno-miR-103-1-5p, rno-miR-103-2-5p, rno-miR-103-3p, rno-miR-105, rno-miR-106b-3p, rno-miR-106b-5p, rno-miR-107-3p, rno-miR-107-5p, rno-miR-10a-3p, rno-miR-10a-5p, rno-miR-10b-3p, rno-miR-10b-5p, rno-miR-1188-3p, rno-miR-1188-5p, rno-miR-1193-3p, rno-miR-1193-5p, rno-miR-1199-3p, rno-miR-1199-5p, rno-miR-122-3p, rno-miR-122-5p, rno-miR-1224, rno-miR-122b, rno-miR-124-3p, rno-miR-124-5p, rno-miR-1247-3p, rno-miR-1247-5p, rno-miR-1249, rno-miR-125a-3p, rno-miR-125a-5p, rno-miR-125b-1-3p, rno-miR-125b-2-3p, rno-miR-125b-5p, rno-miR-126a-3p, rno-miR-126a-5p, rno-miR-126b, rno-miR-127-3p, rno-miR-127-5p, rno-miR-128-1-5p, rno-miR-128-2-5p, rno-miR-128-3p, rno-miR-129-1-3p, rno-miR-129-2-3p, rno-miR-129-5p, rno-miR-1297, rno-miR-1298, rno-miR-1306-3p, rno-miR-1306-5p, rno-miR-130a-3p, rno-miR-130a-5p, rno-miR-130b-3p, rno-miR-130b-5p, rno-miR-132-3p, rno-miR-132-5p, rno-miR-133a-3p, rno-miR-133a-5p, rno-miR-133b-3p, rno-miR-133b-5p, rno-miR-133c, rno-miR-134-3p, rno-miR-134-5p, rno-miR-135a-3p, rno-miR-135a-5p, rno-miR-135b-3p, rno-miR-135b-5p, rno-miR-136-3p, rno-miR-136-5p, rno-miR-137-3p, rno-miR-137-5p, rno-miR-138-1-3p, rno-miR-138-2-3p, rno-miR-138-5p, rno-miR-139-3p, rno-miR-139-5p, rno-miR-140-3p, rno-miR-140-5p, rno-miR-141-3p, rno-miR-141-5p, rno-miR-142-3p, rno-miR-142-5p, rno-miR-143-3p, rno-miR-143-5p, rno-miR-144-3p, rno-miR-144-5p, rno-miR-145-3p, rno-miR-145-5p, rno-miR-146a-3p, rno-miR-146a-5p, rno-miR-146b-3p, rno-miR-146b-5p, rno-miR-147, rno-miR-148a-3p, rno-miR-148a-5p, rno-miR-148b-3p, rno-miR-148b-5p, rno-miR-149-3p, rno-miR-149-5p, rno-miR-150-3p, rno-miR-150-5p, rno-miR-151-3p, rno-miR-151-5p, rno-miR-152-3p, rno-miR-152-5p, rno-miR-153-3p, rno-miR-153-5p, rno-miR-154-3p, rno-miR-154-5p, rno-miR-155-3p, rno-miR-155-5p, rno-miR-15a-3p, rno-miR-15a-5p, rno-miR-15b-3p, rno-miR-15b-5p, rno-miR-16-3p, rno-miR-16-5p, rno-miR-17-1-3p, rno-miR-17-2-3p, rno-miR-17-5p, rno-miR-181a-1-3p, rno-miR-181a-2-3p, rno-miR-181a-5p, rno-miR-181b-1-3p, rno-miR-181b-2-3p, rno-miR-181b-5p, rno-miR-181c-3p, rno-miR-181c-5p, rno-miR-181d-3p, rno-miR-181d-5p, rno-miR-182, rno-miR-183-3p, rno-miR-183-5p, rno-miR-1839-3p, rno-miR-1839-5p, rno-miR-184, rno-miR-1843a-3p, rno-miR-1843a-5p, rno-miR-1843b-3p, rno-miR-1843b-5p, rno-miR-185-3p, rno-miR-185-5p, rno-miR-186-3p, rno-miR-186-5p, rno-miR-187-3p, rno-miR-187-5p, rno-miR-188-3p, rno-miR-188-5p, rno-miR-1896, rno-miR-18a-3p, rno-miR-18a-5p, rno-miR-190a-3p, rno-miR-190a-5p, rno-miR-190b-3p, rno-miR-190b-5p, rno-miR-1912-3p, rno-miR-1912-5p, rno-miR-191a-3p, rno-miR-191a-5p, rno-miR-191b, rno-miR-192-3p, rno-miR-192-5p, rno-miR-193a-3p, rno-miR-193a-5p, rno-miR-193b-3p, rno-miR-193b-5p, rno-miR-194-3p, rno-miR-194-5p, rno-miR-1949, rno-miR-195-3p, rno-miR-195-5p, rno-miR-1956-3p, rno-miR-1956-5p, rno-miR-196a-3p, rno-miR-196a-5p, rno-miR-196b-3p, rno-miR-196b-5p, rno-miR-196c-3p, rno-miR-196c-5p, rno-miR-199a-3p, rno-miR-199a-5p, rno-miR-19a-3p, rno-miR-19a-5p, rno-miR-19b-1-5p, rno-miR-19b-2-5p, rno-miR-19b-3p, rno-miR-1b, rno-miR-200a-3p, rno-miR-200a-5p, rno-miR-200b-3p, rno-miR-200b-5p, rno-miR-200c-3p, rno-miR-200c-5p, rno-miR-201-3p, rno-miR-201-5p, rno-miR-202-3p, rno-miR-202-5p, rno-miR-203a-3p, rno-miR-203a-5p, rno-miR-203b-3p, rno-miR-203b-5p, rno-miR-204-3p, rno-miR-204-5p, rno-miR-205, rno-miR-206-3p, rno-miR-206-5p, rno-miR-207, rno-miR-208a-3p, rno-miR-208a-5p, rno-miR-208b-3p, rno-miR-208b-5p, rno-miR-20a-3p, rno-miR-20a-5p, rno-miR-20b-3p, rno-miR-20b-5p, rno-miR-21-3p, rno-miR-21-5p, rno-miR-210-3p, rno-miR-210-5p, rno-miR-211-3p, rno-miR-211-5p, rno-miR-212-3p, rno-miR-212-5p, rno-miR-214-3p, rno-miR-214-5p, rno-miR-215, rno-miR-216a-3p, rno-miR-216a-5p, rno-miR-216b-3p, rno-miR-216b-5p, rno-miR-217-3p, rno-miR-217-5p, rno-miR-218a-1-3p, rno-miR-218a-2-3p, rno-miR-218a-5p, rno-miR-218b, rno-miR-219a-1-3p, rno-miR-219a-2-3p, rno-miR-219a-5p, rno-miR-219b, rno-miR-22-3p, rno-miR-22-5p, rno-miR-221-3p, rno-miR-221-5p, rno-miR-222-3p, rno-miR-222-5p, rno-miR-223-3p, rno-miR-223-5p, rno-miR-224-3p, rno-miR-224-5p, rno-miR-23a-3p, rno-miR-23a-5p, rno-miR-23b-3p, rno-miR-23b-5p, rno-miR-24-1-5p, rno-miR-24-2-5p, rno-miR-24-3p, rno-miR-25-3p, rno-miR-25-5p, rno-miR-26a-3p, rno-miR-26a-5p, rno-miR-26b-3p, rno-miR-26b-5p, rno-miR-27a-3p, rno-miR-27a-5p, rno-miR-27b-3p, rno-miR-27b-5p, rno-miR-28-3p, rno-miR-28-5p, rno-miR-290, rno-miR-291a-3p, rno-miR-291a-5p, rno-miR-291b, rno-miR-292-3p, rno-miR-292-5p, rno-miR-293-3p, rno-miR-293-5p, rno-miR-294, rno-miR-295-3p, rno-miR-295-5p, rno-miR-296-3p, rno-miR-296-5p, rno-miR-297, rno-miR-298-3p, rno-miR-298-5p, rno-miR-2985, rno-miR-299a-3p, rno-miR-299a-5p, rno-miR-299b-3p, rno-miR-299b-5p, rno-miR-29a-3p, rno-miR-29a-5p, rno-miR-29b-1-5p, rno-miR-29b-3p, rno-miR-29b-5p, rno-miR-29c-3p, rno-miR-29c-5p, rno-miR-300-3p, rno-miR-300-5p, rno-miR-301a-3p, rno-miR-301a-5p, rno-miR-301b-3p, rno-miR-301b-5p, rno-miR-3064-3p, rno-miR-3064-5p, rno-miR-3065-3p, rno-miR-3065-5p, rno-miR-3068-3p, rno-miR-3068-5p, rno-miR-3072, rno-miR-3074, rno-miR-3075, rno-miR-3084a-1-5p, rno-miR-3084a-3p, rno-miR-3084a-5p, rno-miR-3084b-3p, rno-miR-3084b-5p, rno-miR-3085, rno-miR-3099, rno-miR-30a-3p, rno-miR-30a-5p, rno-miR-30b-3p, rno-miR-30b-5p, rno-miR-30c-1-3p, rno-miR-30c-2-3p, rno-miR-30c-5p, rno-miR-30d-3p, rno-miR-30d-5p, rno-miR-30e-3p, rno-miR-30e-5p, rno-miR-3102, rno-miR-3120, rno-miR-31a-3p, rno-miR-31a-5p, rno-miR-31b, rno-miR-32-3p, rno-miR-32-5p, rno-miR-320-3p, rno-miR-320-5p, rno-miR-322-3p, rno-miR-322-5p, rno-miR-323-3p, rno-miR-323-5p, rno-miR-324-3p, rno-miR-324-5p, rno-miR-325-3p, rno-miR-325-5p, rno-miR-326-3p, rno-miR-326-5p, rno-miR-327, rno-miR-328a-3p, rno-miR-328a-5p, rno-miR-328b-3p, rno-miR-329-3p, rno-miR-329-5p, rno-miR-33-3p, rno-miR-33-5p, rno-miR-330-3p, rno-miR-330-5p, rno-miR-331-3p, rno-miR-331-5p, rno-miR-335, rno-miR-336-3p, rno-miR-336-5p, rno-miR-337-3p, rno-miR-337-5p, rno-miR-338-3p, rno-miR-338-5p, rno-miR-339-3p, rno-miR-339-5p, rno-miR-340-3p, rno-miR-340-5p, rno-miR-341, rno-miR-342-3p, rno-miR-342-5p, rno-miR-343, rno-miR-344a-3p, rno-miR-344a-5p, rno-miR-344b-1-3p, rno-miR-344b-3p, rno-miR-344b-5p, rno-miR-344g, rno-miR-344i, rno-miR-345-3p, rno-miR-345-5p, rno-miR-346, rno-miR-347, rno-miR-3473, rno-miR-349, rno-miR-34a-3p, rno-miR-34a-5p, rno-miR-34b-3p, rno-miR-34b-5p, rno-miR-34c-3p, rno-miR-34c-5p, rno-miR-350, rno-miR-351-3p, rno-miR-351-5p, rno-miR-352, rno-miR-3541, rno-miR-3542, rno-miR-3543, rno-miR-3544, rno-miR-3546, rno-miR-3547, rno-miR-3548, rno-miR-3549, rno-miR-3550, rno-miR-3551-3p, rno-miR-3551-5p, rno-miR-3552, rno-miR-3553, rno-miR-3556a, rno-miR-3556b, rno-miR-3557-3p, rno-miR-3557-5p, rno-miR-3558-3p, rno-miR-3558-5p, rno-miR-3559-3p, rno-miR-3559-5p, rno-miR-3560, rno-miR-3561-3p, rno-miR-3561-5p, rno-miR-3562, rno-miR-3564, rno-miR-3566, rno-miR-3568, rno-miR-3569, rno-miR-3570, rno-miR-3571, rno-miR-3572, rno-miR-3573-3p, rno-miR-3573-5p, rno-miR-3574, rno-miR-3575, rno-miR-3576, mo-miR-3577, rno-miR-3578, rno-miR-3579, rno-miR-3580-3p, rno-miR-3580-5p, rno-miR-3583-3p, rno-miR-3583-5p, rno-miR-3584-3p, rno-miR-3584-5p, rno-miR-3585-3p, rno-miR-3585-5p, rno-miR-3586-3p, rno-miR-3586-5p, rno-miR-3587, rno-miR-3588, rno-miR-3589, rno-miR-3590-3p, rno-miR-3590-5p, rno-miR-3592, rno-miR-3593-3p, rno-miR-3593-5p, rno-miR-3594-3p, rno-miR-3594-5p, rno-miR-3595, rno-miR-3596a, rno-miR-3596b, rno-miR-3596c, rno-miR-3596d, rno-miR-361-3p, rno-miR-361-5p, rno-miR-362-3p, rno-miR-362-5p, rno-miR-363-3p, rno-miR-363-5p, rno-miR-365-3p, rno-miR-365-5p, rno-miR-369-3p, rno-miR-369-5p, rno-miR-370-3p, rno-miR-370-5p, rno-miR-374-3p, rno-miR-374-5p, rno-miR-375-3p, rno-miR-375-5p, rno-miR-376a-3p, rno-miR-376a-5p, rno-miR-376b-3p, rno-miR-376b-5p, rno-miR-376c-3p, rno-miR-376c-5p, rno-miR-377-3p, rno-miR-377-5p, rno-miR-378a-3p, rno-miR-378a-5p, rno-miR-378b, rno-miR-379-3p, rno-miR-379-5p, rno-miR-380-3p, rno-miR-380-5p, rno-miR-381-3p, rno-miR-381-5p, rno-miR-382-3p, rno-miR-382-5p, rno-miR-383-3p, rno-miR-383-5p, rno-miR-384-3p, rno-miR-384-5p, rno-miR-409a-3p, rno-miR-409a-5p, rno-miR-409b, rno-miR-410-3p, rno-miR-410-5p, rno-miR-411-3p, rno-miR-411-5p, rno-miR-412-3p, rno-miR-412-5p, rno-miR-421-3p, rno-miR-421-5p, rno-miR-423-3p, rno-miR-423-5p, rno-miR-425-3p, rno-miR-425-5p, rno-miR-429, rno-miR-431, rno-miR-433-3p, rno-miR-433-5p, rno-miR-434-3p, rno-miR-434-5p, rno-miR-448-3p, rno-miR-448-5p, rno-miR-449a-3p, rno-miR-449a-5p, rno-miR-449c-3p, rno-miR-449c-5p, rno-miR-450a-3p, rno-miR-450a-5p, rno-miR-450b-3p, rno-miR-450b-5p, rno-miR-451-3p, rno-miR-451-5p, rno-miR-452-3p, rno-miR-452-5p, rno-miR-455-3p, rno-miR-455-5p, rno-miR-463-3p, rno-miR-463-5p, rno-miR-465-3p, rno-miR-465-5p, rno-miR-466b-2-3p, rno-miR-466b-3p, rno-miR-466b-4-3p, rno-miR-466b-5p, rno-miR-466c-3p, rno-miR-466c-5p, rno-miR-466d, rno-miR-471-3p, rno-miR-471-5p, rno-miR-483-3p, rno-miR-483-5p, rno-miR-484, rno-miR-485-3p, rno-miR-485-5p, rno-miR-486, rno-miR-487b-3p, rno-miR-487b-5p, rno-miR-488-3p, rno-miR-488-5p, rno-miR-489-3p, rno-miR-489-5p, rno-miR-490-3p, rno-miR-490-5p, rno-miR-493-3p, rno-miR-493-5p, rno-miR-494-3p, rno-miR-494-5p, rno-miR-495, rno-miR-496-3p, rno-miR-496-5p, rno-miR-497-3p, rno-miR-497-5p, rno-miR-499-3p, rno-miR-499-5p, rno-miR-500-3p, rno-miR-500-5p, rno-miR-501-3p, rno-miR-501-5p, rno-miR-503-3p, rno-miR-503-5p, rno-miR-504, rno-miR-505-3p, rno-miR-505-5p, rno-miR-509-3p, rno-miR-509-5p, rno-miR-511-3p, rno-miR-511-5p, rno-miR-5132-3p, rno-miR-5132-5p, rno-miR-532-3p, rno-miR-532-5p, rno-miR-539-3p, rno-miR-539-5p, rno-miR-540-3p, rno-miR-540-5p, rno-miR-541-3p, rno-miR-541-5p, rno-miR-542-3p, rno-miR-542-5p, rno-miR-543-3p, rno-miR-543-5p, rno-miR-544-3p, rno-miR-544-5p, rno-miR-547-3p, rno-miR-547-5p, rno-miR-551b-3p, rno-miR-551b-5p, rno-miR-568, rno-miR-582-3p, rno-miR-582-5p, rno-miR-592, rno-miR-598-3p, rno-miR-598-5p, rno-miR-615, rno-miR-6215, rno-miR-6216, rno-miR-628, rno-miR-6314, rno-miR-6315, rno-miR-6316, rno-miR-6317, rno-miR-6318, rno-miR-6319, rno-miR-632, rno-miR-6320, rno-miR-6321, rno-miR-6322, rno-miR-6323, rno-miR-6324, rno-miR-6325, rno-miR-6326, rno-miR-6327, rno-miR-6328, rno-miR-6329, rno-miR-6330, rno-miR-6331, rno-miR-6332, rno-miR-6333, rno-miR-6334, rno-miR-652-3p, rno-miR-652-5p, rno-miR-653-3p, rno-miR-653-5p, rno-miR-664-1-5p, rno-miR-664-2-5p, rno-miR-664-3p, rno-miR-665, rno-miR-666-3p, rno-miR-666-5p, rno-miR-667-3p, rno-miR-667-5p, rno-miR-668, rno-miR-671, rno-miR-672-3p, rno-miR-672-5p, rno-miR-673-3p, rno-miR-673-5p, rno-miR-674-3p, rno-miR-674-5p, rno-miR-675-3p, rno-miR-675-5p, rno-miR-676, rno-miR-678, rno-miR-679, rno-miR-702-3p, rno-miR-702-5p, rno-miR-708-3p, rno-miR-708-5p, rno-miR-709, rno-miR-711, rno-miR-741-3p, rno-miR-741-5p, rno-miR-742-3p, rno-miR-742-5p, rno-miR-743a-3p, rno-miR-743a-5p, rno-miR-743b-3p, rno-miR-743b-5p, rno-miR-7578, rno-miR-758-3p, rno-miR-758-5p, rno-miR-759, rno-miR-760-3p, rno-miR-760-5p, rno-miR-761, rno-miR-762, rno-miR-764-3p, rno-miR-764-5p, rno-miR-770-3p, rno-miR-770-5p, rno-miR-7a-1-3p, rno-miR-7a-2-3p, rno-miR-7a-5p, rno-miR-7b, rno-miR-802-3p, rno-miR-802-5p, rno-miR-871-3p, rno-miR-871-5p, rno-miR-872-3p, rno-miR-872-5p, rno-miR-873-3p, rno-miR-873-5p, rno-miR-874-3p, rno-miR-874-5p, rno-miR-875, rno-miR-876, rno-miR-877, rno-miR-878, rno-miR-879-3p, rno-miR-879-5p, rno-miR-880-3p, rno-miR-880-5p, rno-miR-881-3p, rno-miR-881-5p, rno-miR-883-3p, rno-miR-883-5p, rno-miR-92a-1-5p, rno-miR-92a-2-5p, rno-miR-92a-3p, rno-miR-92b-3p, rno-miR-92b-5p, rno-miR-93-3p, rno-miR-93-5p, rno-miR-935, rno-miR-96-3p, rno-miR-96-5p, rno-miR-98-3p, rno-miR-98-5p, rno-miR-99a-3p, rno-miR-99a-5p, rno-miR-99b-3p, rno-miR-99b-5p, rno-miR-9a-3p, rno-miR-9a-5p, rno-miR-9b-3p, rno-miR-9b-5p, rrv-miR-rR1-1-3p, rrv-miR-rR1-1-5p, rrv-miR-rR1-2, rrv-miR-rR1-3, rrv-miR-rR1-4-3p, rrv-miR-rR1-4-5p, rrv-miR-rR1-5, rrv-miR-rR1-6-3p, rrv-miR-rR1-6-5p, rrv-miR-rR1-7-3p, rrv-miR-rR1-7-5p, sbi-miR1432, sbi-miR1435a, sbi-miR1435b, sbi-miR156a, sbi-miR156b, sbi-miR156c, sbi-miR156d, sbi-miR156e, sbi-miR156f, sbi-miR156g, sbi-miR156h, sbi-miR156i, sbi-miR159a, sbi-miR159b, sbi-miR160a, sbi-miR160b, sbi-miR160c, sbi-miR160d, sbi-miR160e, sbi-miR160f, sbi-miR162, sbi-miR164a, sbi-miR164b, sbi-miR164c, sbi-miR164d, sbi-miR164e, sbi-miR166a, sbi-miR166b, sbi-miR166c, sbi-miR166d, sbi-miR166e, sbi-miR166f, sbi-miR166g, sbi-miR166h, sbi-miR166i, sbi-miR166j, sbi-miR166k, sbi-miR167a, sbi-miR167b, sbi-miR167c, sbi-miR167d, sbi-miR167e, sbi-miR167f, sbi-miR167g, sbi-miR167h, sbi-miR167i, sbi-miR168, sbi-miR169a, sbi-miR169b, sbi-miR169c, sbi-miR169d-3p, sbi-miR169d-5p, sbi-miR169e, sbi-miR169f, sbi-miR169g, sbi-miR169h, sbi-miR169i, sbi-miR169j, sbi-miR169k, sbi-miR1691, sbi-miR169m, sbi-miR169n, sbi-miR1690, sbi-miR169p, sbi-miR169q, sbi-miR171a, sbi-miR171b, sbi-miR171c, sbi-miR171d, sbi-miR171e, sbi-miR171f, sbi-miR171g, sbi-miR171h, sbi-miR171i, sbi-miR171j, sbi-miR171k, sbi-miR172a, sbi-miR172b, sbi-miR172c, sbi-miR172d, sbi-miR172e, sbi-miR172f, sbi-miR2118-3p, sbi-miR2118-5p, sbi-miR319a, sbi-miR319b, sbi-miR390, sbi-miR393a, sbi-miR393b, sbi-miR394a, sbi-miR394b, sbi-miR395a, sbi-miR395b, sbi-miR395c, sbi-miR395d, sbi-miR395e, sbi-miR395f, sbi-miR395g, sbi-miR395h, sbi-miR395i, sbi-miR395j, sbi-miR395k, sbi-miR3951, sbi-miR396a, sbi-miR396b, sbi-miR396c, sbi-miR396d, sbi-miR396e, sbi-miR397-3p, sbi-miR397-5p, sbi-miR398, sbi-miR399a, sbi-miR399b, sbi-miR399c, sbi-miR399d, sbi-miR399e, sbi-miR399f, sbi-miR399g, sbi-miR399h, sbi-miR399i, sbi-miR399j, sbi-miR399k, sbi-miR408, sbi-miR437a, sbi-miR437b, sbi-miR437c, sbi-miR437d, sbi-miR437e, sbi-miR437f, sbi-miR437g, sbi-miR437i, sbi-miR437j, sbi-miR437k, sbi-miR437l, sbi-miR437m, sbi-miR437n, sbi-miR437o, sbi-miR437p, sbi-miR437q, sbi-miR437r, sbi-miR437s, sbi-miR437t, sbi-miR437u, sbi-miR437v, sbi-miR437w, sbi-miR437x-3p, sbi-miR437x-5p, sbi-miR528, sbi-miR529, sbi-miR5381, sbi-miR5382, sbi-miR5383, sbi-miR5384, sbi-miR5385, sbi-miR5386, sbi-miR5387a, sbi-miR5387b, sbi-miR5388, sbi-miR5389, sbi-miR5564a, sbi-miR5564b, sbi-miR5564c-3p, sbi-miR5564c-5p, sbi-miR5565a, sbi-miR5565b, sbi-miR5565c, sbi-miR5565d, sbi-miR5565e, sbi-miR5565f, sbi-miR5565g-3p, sbi-miR5565g-5p, sbi-miR5566, sbi-miR5567, sbi-miR5568a, sbi-miR5568b-3p, sbi-miR5568b-5p, sbi-miR5568c-3p, sbi-miR5568c-5p, sbi-miR5568d-3p, sbi-miR5568d-5p, sbi-miR5568e-3p, sbi-miR5568e-5p, sbi-miR5568f-3p, sbi-miR5568f-5p, sbi-miR5568g-3p, sbi-miR5568g-5p, sbi-miR5569, sbi-miR5570, sbi-miR6217a-3p, sbi-miR6217a-5p, sbi-miR6217b-3p, sbi-miR6217b-5p, sbi-miR6218-3p, sbi-miR6218-5p, sbi-miR6219-3p, sbi-miR6219-5p, sbi-miR6220-3p, sbi-miR6220-5p, sbi-miR6221-3p, sbi-miR6221-5p, sbi-miR6222-3p, sbi-miR6222-5p, sbi-miR6223-3p, sbi-miR6223-5p, sbi-miR6224a-3p, sbi-miR6224a-5p, sbi-miR6224b-3p, sbi-miR6224b-5p, sbi-miR6224c-3p, sbi-miR6224c-5p, sbi-miR6225-3p, sbi-miR6225-5p, sbi-miR6226-3p, sbi-miR6226-5p, sbi-miR6227-3p, sbi-miR6227-5p, sbi-miR6228-3p, sbi-miR6228-5p, sbi-miR6229-3p, sbi-miR6229-5p, sbi-miR6230-3p, sbi-miR6230-5p, sbi-miR6231-3p, sbi-miR6231-5p, sbi-miR6232a-3p, sbi-miR6232a-5p, sbi-miR6232b-3p, sbi-miR6232b-5p, sbi-miR6233-3p, sbi-miR6233-5p, sbi-miR6234a-3p, sbi-miR6234a-5p, sbi-miR6234b-3p, sbi-miR6234b-5p, sbi-miR6235-3p, sbi-miR6235-5p, sbi-miR821a, sbi-miR821b, sbi-miR821c, sbi-miR821d, sbi-miR821e, sbo-let-7a, sbo-let-7b, sbo-let-7e, sbo-let-7f, sbo-miR-100, sbo-miR-10a, sbo-miR-10b, sbo-miR-125a, sbo-miR-125b, sbo-miR-127, sbo-miR-143, sbo-miR-146b, sbo-miR-148a, sbo-miR-151, sbo-miR-16, sbo-miR-181a, sbo-miR-186, sbo-miR-191, sbo-miR-199, sbo-miR-21, sbo-miR-214, sbo-miR-22, sbo-miR-221, sbo-miR-24, sbo-miR-26a, sbo-miR-27a, sbo-miR-27b, sbo-miR-28, sbo-miR-29a, sbo-miR-423, sbo-miR-424, sbo-miR-486a, sbo-miR-486b, sbo-miR-6529, sbo-miR-92a, sbo-miR-99a, sci-miR-7642-3p, sci-miR-7642-5p, seu-miR11021, seu-miR11022, seu-miR11023, seu-miR11024, seu-miR11025, seu-miR11026, seu-miR11027, seu-miR11028, seu-miR11029, seu-miR11030a, seu-miR11030b, seu-miR11031, seu-miR11032, seu-miR11033, seu-miR11034, seu-miR11035, seu-miR11036, seu-miR11037, seu-miR11038, seu-miR11039, seu-miR11040, seu-miR11041, seu-miR11042, seu-miR11043, seu-miR11044, seu-miR11045, seu-miR11046, seu-miR11047, seu-miR11048, seu-miR11049, seu-miR319, sfr-miR-10-3p, sfr-miR-10-5p, sfr-miR-10451-3p, sfr-miR-10451-5p, sfr-miR-10452-3p, sfr-miR-10452-5p, sfr-miR-10453-3p, sfr-miR-10453-5p, sfr-miR-10454a-2-5p, sfr-miR-10454a-3p, sfr-miR-10454a-5p, sfr-miR-10454b-3p, sfr-miR-10454b-5p, sfr-miR-10454c-3p, sfr-miR-10454c-5p, sfr-miR-10454d-3p, sfr-miR-10454d-5p, sfr-miR-10454e-3p, sfr-miR-10454e-5p, sfr-miR-10454f-3p, sfr-miR-10454f-5p, sfr-miR-10454g-3p, sfr-miR-10454g-5p, sfr-miR-10454 h-3p, sfr-miR-10454 h-5p, sfr-miR-10455-3p, sfr-miR-10455-5p, sfr-miR-10456-3p, sfr-miR-10456-5p, sfr-miR-10457-3p, sfr-miR-10457-5p, sfr-miR-10458-3p, sfr-miR-10458-5p, sfr-miR-10459-3p, sfr-miR-10459-5p, sfr-miR-10460-3p, sfr-miR-10460-5p, sfr-miR-10461a-3p, sfr-miR-10461a-5p, sfr-miR-10461b-3p, sfr-miR-10461b-5p, sfr-miR-10462-3p, sfr-miR-10462-5p, sfr-miR-10463-3p, sfr-miR-10463-5p, sfr-miR-10464-3p, sfr-miR-10464-5p, sfr-miR-10465-2-3p, sfr-miR-10465-3p, sfr-miR-10465-5p, sfr-miR-10466-3p, sfr-miR-10466-5p, sfr-miR-10467-3p, sfr-miR-10467-5p, sfr-miR-10468-3p, sfr-miR-10468-5p, sfr-miR-10469-3p, sfr-miR-10469-5p, sfr-miR-10470-3p, sfr-miR-10470-5p, sfr-miR-10471-3p, sfr-miR-10471-5p, sfr-miR-10472-3p, sfr-miR-10472-5p, sfr-miR-10473-3p, sfr-miR-10473-5p, sfr-miR-10474-3p, sfr-miR-10474-5p, sfr-miR-10475-3p, sfr-miR-10475-5p, sfr-miR-10476-3p, sfr-miR-10476-5p, sfr-miR-10477-3p, sfr-miR-10477-5p, sfr-miR-10478-3p, sfr-miR-10478-5p, sfr-miR-10479-3p, sfr-miR-10479-5p, sfr-miR-10480-3p, sfr-miR-10480-5p, sfr-miR-10481-3p, sfr-miR-10481-5p, sfr-miR-10482-3p, sfr-miR-10482-5p, sfr-miR-10483-2-3p, sfr-miR-10483-3p, sfr-miR-10483-5p, sfr-miR-10484-??, sfr-miR-10484-3p, sfr-miR-10485-3p, sfr-miR-10485-5p, sfr-miR-10486-3p, sfr-miR-10486-5p, sfr-miR-10487-3p, sfr-miR-10487-5p, sfr-miR-10488-3p, sfr-miR-10488-5p, sfr-miR-10489-3p, sfr-miR-10489-5p, sfr-miR-10490-3p, sfr-miR-10490-5p, sfr-miR-10491-3p, sfr-miR-10491-5p, sfr-miR-10492a-3p, sfr-miR-10492a-5p, sfr-miR-10492b-3p, sfr-miR-10492b-5p, sfr-miR-10492c-3p, sfr-miR-10492c-5p, sfr-miR-10493-3p, sfr-miR-10493-5p, sfr-miR-10494-3p, sfr-miR-10494-5p, sfr-miR-10495-3-3p, sfr-miR-10495-3p, sfr-miR-10495-5p, sfr-miR-10496-3p, sfr-miR-10496-5p, sfr-miR-10497-3p, sfr-miR-10497-5p, sfr-miR-10498-3p, sfr-miR-10498-5p, sfr-miR-10499a-3p, sfr-miR-10499a-4-5p, sfr-miR-10499a-5p, sfr-miR-10499b-2-5p, sfr-miR-10499b-3p, sfr-miR-10499b-5p, sfr-miR-10500-3p, sfr-miR-10500-5p, sfr-miR-10501-3p, sfr-miR-10501-5p, sfr-miR-10502-3p, sfr-miR-10502-5p, sfr-miR-10503-3p, sfr-miR-10503-5p, sfr-miR-10504-3p, sfr-miR-10504-5p, sfr-miR-10505-3p, sfr-miR-10505-5p, sfr-miR-10506-3p, sfr-miR-10506-5p, sfr-miR-10507-3p, sfr-miR-10507-5p, sfr-miR-10508a-2-3p, sfr-miR-10508a-3p, sfr-miR-10508a-5p, sfr-miR-10508b-3p, sfr-miR-10508b-5p, sfr-miR-10509-3p, sfr-miR-10509-5p, sfr-miR-10510-3p, sfr-miR-10510-5p, sfr-miR-10511-3p, sfr-miR-10511-5p, sfr-miR-10512-3p, sfr-miR-10512-5p, sfr-miR-10513-3p, sfr-miR-10513-5p, sfr-miR-10514-3p, sfr-miR-10514-5p, sfr-miR-11-3p, sfr-miR-11-5p, sfr-miR-13a-3p, sfr-miR-13a-5p, sfr-miR-13b-3p, sfr-miR-13b-5p, sfr-miR-14-3p, sfr-miR-14-5p, sfr-miR-210-3p, sfr-miR-210-5p, sfr-miR-263b-3p, sfr-miR-263b-5p, sfr-miR-274-3p, sfr-miR-274-5p, sfr-miR-2755-3p, sfr-miR-2755-5p, sfr-miR-2756-3p, sfr-miR-2756-5p, sfr-miR-2765-3p, sfr-miR-2765-5p, sfr-miR-2766-3p, sfr-miR-2766-5p, sfr-miR-277-3p, sfr-miR-277-5p, sfr-miR-279a-3p, sfr-miR-279a-5p, sfr-miR-279b-3p, sfr-miR-279b-5p, sfr-miR-279c-3p, sfr-miR-279c-5p, sfr-miR-281-3p, sfr-miR-281-5p, sfr-miR-285-3p, sfr-miR-285-5p, sfr-miR-2a-3p, sfr-miR-2a-5p, sfr-miR-2b-3p, sfr-miR-2b-5p, sfr-miR-2c-3p, sfr-miR-2c-5p, sfr-miR-307-3p, sfr-miR-307-5p, sfr-miR-316-3p, sfr-miR-316-5p, sfr-miR-317-3p, sfr-miR-317-5p, sfr-miR-3286-3p, sfr-miR-3286-5p, sfr-miR-34-3p, sfr-miR-34-5p, sfr-miR-375-3p, sfr-miR-375-5p, sfr-miR-6094-3p, sfr-miR-6094-5p, sfr-miR-7-3p, sfr-miR-7-5p, sfr-miR-92a-3p, sfr-miR-92a-5p, sfr-miR-932-3p, sfr-miR-932-5p, sfv-miR-S1-3p, sfv-miR-S2-3p, sfv-miR-S2-5p, sfv-miR-S3-3p, sfv-miR-S3-5p, sfv-miR-S4-3p, sfv-miR-S4-5p, sfv-miR-S5-3p, sfv-miR-S5-5p, sfv-miR-S6-3p, sfv-miR-S6-5p, sfv-miR-S7-3p, sfv-miR-S7-5p, sha-let-7a, sha-let-7g, sha-let-7i, sha-miR-100, sha-miR-101, sha-miR-10a, sha-miR-10b, sha-miR-125a, sha-miR-125b, sha-miR-126, sha-miR-128, sha-miR-129, sha-miR-130a, sha-miR-130b, sha-miR-1329, sha-miR-133a, sha-miR-135a, sha-miR-138, sha-miR-139, sha-miR-140, sha-miR-141, sha-miR-142, sha-miR-150, sha-miR-1540, sha-miR-1546, sha-miR-1549, sha-miR-181a-3p, sha-miR-181a-5p, sha-miR-190, sha-miR-193, sha-miR-199a, sha-miR-19b, sha-miR-200a, sha-miR-200b, sha-miR-200c, sha-miR-202, sha-miR-204, sha-miR-205, sha-miR-21, sha-miR-216a, sha-miR-216b, sha-miR-221, sha-miR-222, sha-miR-23a, sha-miR-23b, sha-miR-24, sha-miR-25, sha-miR-26a, sha-miR-27b, sha-miR-30c, sha-miR-30e, sha-miR-338, sha-miR-340, sha-miR-352, sha-miR-363, sha-miR-383, sha-miR-454, sha-miR-497, sha-miR-7, sha-miR-9, sha-miR-92a, sha-miR-93, sja-bantam, sja-let-7, sja-miR-1, sja-miR-10-3p, sja-miR-10-5p, sja-miR-124-3p, sja-miR-124-5p, sja-miR-125a, sja-miR-125b, sja-miR-133, sja-miR-190-3p, sja-miR-190-5p, sja-miR-2162-3p, sja-miR-2162-5p, sja-miR-219-3p, sja-miR-219-5p, sja-miR-277, sja-miR-2a-3p, sja-miR-2a-5p, sja-miR-2b-3p, sja-miR-2b-5p, sja-miR-2c-3p, sja-miR-2c-5p, sja-miR-2d-3p, sja-miR-2d-5p, sja-miR-2e-3p, sja-miR-2e-5p, sja-miR-307, sja-miR-31-3p, sja-miR-31-5p, sja-miR-310, sja-miR-3479-3p, sja-miR-3479-5p, sja-miR-3480-3p, sja-miR-3480-5p, sja-miR-3481-3p, sja-miR-3481-5p, sja-miR-3482-3p, sja-miR-3482-5p, sja-miR-3483-3p, sja-miR-3483-5p, sja-miR-3484-3p, sja-miR-3484-5p, sja-miR-3485-3p, sja-miR-3485-5p, sja-miR-3486-3p, sja-miR-3486-5p, sja-miR-3487, sja-miR-3488, sja-miR-3489, sja-miR-3490, sja-miR-3491, sja-miR-3492, sja-miR-3493, sja-miR-3494, sja-miR-3495, sja-miR-3496, sja-miR-3497, sja-miR-3498, sja-miR-3499, sja-miR-3500, sja-miR-3501, sja-miR-3502, sja-miR-3503, sja-miR-3504, sja-miR-3505, sja-miR-3506, sja-miR-3507, sja-miR-36-3p, sja-miR-36-5p, sja-miR-61, sja-miR-7-3p, sja-miR-7-5p, sja-miR-71a, sja-miR-71b-3p, sja-miR-71b-5p, sja-miR-8-3p, sja-miR-8-5p, sja-miR-8185, sko-let-7, sko-miR-1-3p, sko-miR-1-5p, sko-miR-10, sko-miR-100, sko-miR-124-3p, sko-miR-124-5p, sko-miR-125a, sko-miR-133, sko-miR-137, sko-miR-153, sko-miR-182, sko-miR-183, sko-miR-184-3p, sko-miR-184-5p, sko-miR-190, sko-miR-193a-3p, sko-miR-193a-5p, sko-miR-193b, sko-miR-200, sko-miR-2001, sko-miR-2006-3p, sko-miR-2006-5p, sko-miR-2007-3p, sko-miR-2007-5p, sko-miR-2008, sko-miR-2011, sko-miR-2012-3p, sko-miR-2012-5p, sko-miR-2013, sko-miR-210-3p, sko-miR-210-5p, sko-miR-219, sko-miR-242, sko-miR-252a, sko-miR-252b, sko-miR-278, sko-miR-281, sko-miR-29a, sko-miR-29b-3p, sko-miR-29b-5p, sko-miR-31, sko-miR-315, sko-miR-33-3p, sko-miR-34-3p, sko-miR-34-5p, sko-miR-365-3p, sko-miR-365-5p, sko-miR-375, sko-miR-4817-5p, sko-miR-4818a, sko-miR-4818b-3p, sko-miR-4818b-5p, sko-miR-4818c, sko-miR-4818d, sko-miR-4818e-3p, sko-miR-4818e-5p, sko-miR-4818f, sko-miR-4818g-3p, sko-miR-4818g-5p, sko-miR-4819-3p, sko-miR-4819-5p, sko-miR-4820, sko-miR-4821, sko-miR-4822-3p, sko-miR-4822-5p, sko-miR-4823-3p, sko-miR-4823-5p, sko-miR-4824, sko-miR-4825-3p, sko-miR-4825-5p, sko-miR-4826-3p, sko-miR-4826-5p, sko-miR-4827a, sko-miR-4827b, sko-miR-4828a, sko-miR-4828b, sko-miR-4829a, sko-miR-4829b, sko-miR-4829c, sko-miR-4830a, sko-miR-4830b-3p, sko-miR-4830b-5p, sko-miR-4830c, sko-miR-4830d-3p, sko-miR-4830d-5p, sko-miR-4831, sko-miR-4832, sko-miR-4833, sko-miR-4834, sko-miR-4835-3p, sko-miR-4835-5p, sko-miR-4836-3p, sko-miR-4836-5p, sko-miR-4837, sko-miR-4838, sko-miR-4839, sko-miR-4840, sko-miR-4841, sko-miR-4842, sko-miR-4843, sko-miR-4844, sko-miR-4845, sko-miR-4846, sko-miR-7-3p, sko-miR-7-5p, sko-miR-71, sko-miR-9-3p, sko-miR-9-5p, sko-miR-92a, sko-miR-92b, sko-miR-92c, sko-miR-96-3p, sko-miR-96-5p, sla-miR-100, sla-miR-101, sla-miR-105, sla-miR-106a, sla-miR-106b, sla-miR-10a, sla-miR-125b, sla-miR-127, sla-miR-128, sla-miR-133a, sla-miR-147, sla-miR-15a, sla-miR-16, sla-miR-17-3p, sla-miR-17-5p, sla-miR-18, sla-miR-181a, sla-miR-183, sla-miR-198, sla-miR-199a, sla-miR-19a, sla-miR-19b, sla-miR-20, sla-miR-204, sla-miR-214, sla-miR-218, sla-miR-22, sla-miR-223, sla-miR-23a, sla-miR-27a, sla-miR-28, sla-miR-29a, sla-miR-29b, sla-miR-32, sla-miR-34a, sla-miR-7, sla-miR-92, sla-miR-93, sla-miR-95, sla-miR-96, sly-miR10528, sly-miR10529, sly-miR10530, sly-miR10531, sly-miR10532, sly-miR10533, sly-miR10534, sly-miR10535a, sly-miR10535b, sly-miR10536, sly-miR10537, sly-miR10538, sly-miR10539, sly-miR10540, sly-miR10541, sly-miR10542, sly-miR156a, sly-miR156b, sly-miR156c, sly-miR156d-3p, sly-miR156d-5p, sly-miR156e-3p, sly-miR156e-5p, sly-miR159, sly-miR159b, sly-miR160a, sly-miR162, sly-miR164a-3p, sly-miR164a-5p, sly-miR164b-3p, sly-miR164b-5p, sly-miR166a, sly-miR166b, sly-miR166c-3p, sly-miR166c-5p, sly-miR167a, sly-miR167b-3p, sly-miR167b-5p, sly-miR168a-3p, sly-miR168a-5p, sly-miR168b-3p, sly-miR168b-5p, sly-miR169a, sly-miR169b, sly-miR169c, sly-miR169d, sly-miR169e-3p, sly-miR169e-5p, sly-miR169f, sly-miR171a, sly-miR171b-3p, sly-miR171b-5p, sly-miR171c, sly-miR171d, sly-miR171e, sly-miR171f, sly-miR172a, sly-miR172b, sly-miR172c, sly-miR172d, sly-miR1916, sly-miR1917, sly-miR1918, sly-miR1919a, sly-miR1919b, sly-miR1919c-3p, sly-miR1919c-5p, sly-miR319a, sly-miR319b, sly-miR319c-3p, sly-miR319c-5p, sly-miR319d, sly-miR390a-3p, sly-miR390a-5p, sly-miR390b-3p, sly-miR390b-5p, sly-miR391, sly-miR393, sly-miR394-3p, sly-miR394-5p, sly-miR395a, sly-miR395b, sly-miR396a-3p, sly-miR396a-5p, sly-miR396b, sly-miR397-3p, sly-miR397-5p, sly-miR398a, sly-miR399, sly-miR399b, sly-miR403-3p, sly-miR403-5p, sly-miR408, sly-miR477-3p, sly-miR477-5p, sly-miR482a, sly-miR482b, sly-miR482c, sly-miR482d-3p, sly-miR482d-5p, sly-miR482e-3p, sly-miR482e-5p, sly-miR530, sly-miR5300, sly-miR5302a, sly-miR5302b-3p, sly-miR5302b-5p, sly-miR5303, sly-miR5304, sly-miR6022, sly-miR6023, sly-miR6024, sly-miR6025, sly-miR6026, sly-miR6027-3p, sly-miR6027-5p, sly-miR7981a, sly-miR7981b, sly-miR7981c, sly-miR7981d, sly-miR7981e, sly-miR7981f, sly-miR827, sly-miR9469-3p, sly-miR9469-5p, sly-miR9470-3p, sly-miR9470-5p, sly-miR9471a-3p, sly-miR9471a-5p, sly-miR9471b-3p, sly-miR9471b-5p, sly-miR9472-3p, sly-miR9472-5p, sly-miR9473-3p, sly-miR9473-5p, sly-miR9474-3p, sly-miR9474-5p, sly-miR9475-3p, sly-miR9475-5p, sly-miR9476-3p, sly-miR9476-5p, sly-miR9477-3p, sly-miR9477-5p, sly-miR9478-3p, sly-miR9478-5p, sly-miR9479-3p, sly-miR9479-5p, sma-bantam-3p, sma-bantam-5p, sma-let-7-3p, sma-let-7-5p, sma-miR-10-3p, sma-miR-10-5p, sma-miR-124a-3p, sma-miR-124a-5p, sma-miR-124b-3p, sma-miR-124b-5p, sma-miR-125a-3p, sma-miR-125a-5p, sma-miR-125b, sma-miR-125c-3p, sma-miR-125c-5p, sma-miR-190-3p, sma-miR-190-5p, sma-miR-1a-5p, sma-miR-1b-3p, sma-miR-1b-5p, sma-miR-2162-3p, sma-miR-2162-5p, sma-miR-219-3p, sma-miR-219-5p, sma-miR-281-3p, sma-miR-281-5p, sma-miR-2a-3p, sma-miR-2a-5p, sma-miR-2b-3p, sma-miR-2b-5p, sma-miR-2c-3p, sma-miR-2c-5p, sma-miR-2d-3p, sma-miR-2d-5p, sma-miR-2e-3p, sma-miR-2e-5p, sma-miR-2f-3p, sma-miR-31-3p, sma-miR-31-5p, sma-miR-3479-3p, sma-miR-3492, sma-miR-36a-3p, sma-miR-36a-5p, sma-miR-36b-3p, sma-miR-36b-5p, sma-miR-61-3p, sma-miR-61-5p, sma-miR-7-3p, sma-miR-7-5p, sma-miR-71a-3p, sma-miR-71a-5p, sma-miR-71b-3p, sma-miR-71b-5p, sma-miR-745-3p, sma-miR-745-5p, sma-miR-755-3p, sma-miR-755-5p, sma-miR-8-3p, sma-miR-8-5p, sma-miR-8403-3p, sma-miR-8403-5p, sma-miR-8404-3p, sma-miR-8404-5p, sma-miR-8405-3p, sma-miR-8405-5p, sma-miR-8406-3p, sma-miR-8406-5p, sma-miR-8407-3p, sma-miR-8407-5p, sma-miR-8408-3p, sma-miR-8408-5p, sma-miR-8409-3p, sma-miR-8409-5p, sma-miR-8410-3p, sma-miR-8410-5p, sma-miR-8411-3p, sma-miR-8411-5p, sma-miR-8412-3p, sma-miR-8412-5p, sma-miR-8413-3p, sma-miR-8413-5p, sma-miR-8414-3p, sma-miR-8414-5p, sma-miR-8415-3p, sma-miR-8415-5p, sma-miR-8416-3p, sma-miR-8416-5p, sma-miR-8417-3p, sma-miR-8417-5p, sma-miR-8418-3p, sma-miR-8418-5p, sma-miR-8419-3p, sma-miR-8419-5p, sma-miR-8420-3p, sma-miR-8420-5p, sma-miR-8421-3p, sma-miR-8421-5p, sma-miR-8422-3p, sma-miR-8422-5p, sma-miR-8423-3p, sma-miR-8423-5p, sma-miR-8424-3p, sma-miR-8424-5p, sma-miR-8425-3p, sma-miR-8425-5p, sma-miR-8426-3p, sma-miR-8426-5p, sma-miR-8427-3p, sma-miR-8427-5p, sma-miR-8428-3p, sma-miR-8428-5p, sma-miR-8429-3p, sma-miR-8429-5p, sma-miR-8430-3p, sma-miR-8430-5p, sma-miR-8431-3p, sma-miR-8431-5p, sma-miR-8432-3p, sma-miR-8432-5p, sma-miR-8433-3p, sma-miR-8433-5p, sma-miR-8434-3p, sma-miR-8434-5p, sma-miR-8435-3p, sma-miR-8435-5p, sma-miR-8436-3p, sma-miR-8436-5p, sma-miR-8437-3p, sma-miR-8437-5p, sma-miR-8438-3p, sma-miR-8438-5p, sma-miR-8439-3p, sma-miR-8439-5p, sma-miR-8440-3p, sma-miR-8440-5p, sma-miR-8441-3p, sma-miR-8441-5p, sma-miR-8442-3p, sma-miR-8442-5p, sma-miR-8443-3p, sma-miR-8443-5p, sma-miR-8444-3p, sma-miR-8444-5p, sma-miR-8445-3p, sma-miR-8445-5p, sma-miR-8446-3p, sma-miR-8446-5p, sma-miR-8447-3p, sma-miR-8447-5p, sma-miR-8448-3p, sma-miR-8448-5p, sma-miR-8449-3p, sma-miR-8449-5p, sma-miR-8450-3p, sma-miR-8450-5p, sma-miR-8451-3p, sma-miR-8451-5p, sma-miR-8452-3p, sma-miR-8452-5p, sma-miR-8453-3p, sma-miR-8453-5p, sma-miR-8454-3p, sma-miR-8454-5p, sma-miR-8455-3p, sma-miR-8455-5p, sma-miR-8456-3p, sma-miR-8456-5p, sma-miR-8457-3p, sma-miR-8457-5p, sma-miR-8458-3p, sma-miR-8458-5p, sma-miR-8459-3p, sma-miR-8459-5p, sma-miR-8460-3p, sma-miR-8460-5p, sma-miR-8461-3p, sma-miR-8461-5p, sma-miR-8462-3p, sma-miR-8462-5p, sma-miR-8463-3p, sma-miR-8463-5p, sma-miR-8464-3p, sma-miR-8464-5p, sma-miR-8465-3p, sma-miR-8465-5p, sma-miR-8466-3p, sma-miR-8466-5p, sma-miR-8467-3p, sma-miR-8467-5p, sma-miR-8468-3p, sma-miR-8468-5p, sma-miR-8469-3p, sma-miR-8469-5p, sma-miR-8470-3p, sma-miR-8470-5p, sma-miR-8471-3p, sma-miR-8471-5p, sma-miR-8472-3p, sma-miR-8472-5p, sma-miR-8473-3p, sma-miR-8473-5p, sma-miR-8474-3p, sma-miR-8474-5p, sma-miR-8475-3p, sma-miR-8475-5p, sma-miR-8476-3p, sma-miR-8476-5p, sma-miR-8477-3p, sma-miR-8477-5p, sma-miR-8478-3p, sma-miR-8478-5p, sma-miR-8479-3p, sma-miR-8479-5p, sma-miR-8480-3p, sma-miR-8480-5p, sma-miR-8481-3p, sma-miR-8481-5p, sma-miR-8482-3p, sma-miR-8482-5p, sma-miR-8483-3p, sma-miR-8483-5p, sma-miR-8484-3p, sma-miR-8484-5p, sma-miR-96-3p, sma-miR-96-5p, smc-miR-12455-2-3p, smc-miR-12455-3p, smc-miR-12455-5p, smc-miR-12456-3p, smc-miR-12456-5p, smc-miR-12457-3p, smc-miR-12457-5p, smc-miR-12458-3p, smc-miR-12458-5p, smc-miR-12459-3p, smc-miR-12459-5p, smc-miR-12460-3p, smc-miR-12460-5p, smc-miR-12461-3p, smc-miR-12461-5p, sme-bantam-a, sme-bantam-b-3p, sme-bantam-b-5p, sme-bantam-c-3p, sme-bantam-c-5p, sme-let-7a-3p, sme-let-7a-5p, sme-let-7b-3p, sme-let-7b-5p, sme-let-7c-3p, sme-let-7c-5p, sme-let-7d, sme-lin-4-3p, sme-lin-4-5p, sme-miR-10a-3p, sme-miR-10a-5p, sme-miR-10b-3p, sme-miR-10b-5p, sme-miR-1175-3p, sme-miR-1175-5p, sme-miR-12-3p, sme-miR-12-5p, sme-miR-124a-3p, sme-miR-124a-5p, sme-miR-124b-3p, sme-miR-124b-5p, sme-miR-124c-1-5p, sme-miR-124c-2-5p, sme-miR-124c-3p, sme-miR-124d-3p, sme-miR-124d-5p, sme-miR-124e-3p, sme-miR-124e-5p, sme-miR-125a-3p, sme-miR-125a-5p, sme-miR-125b-3p, sme-miR-125b-5p, sme-miR-13-3p, sme-miR-13-5p, sme-miR-133a-3p, sme-miR-133a-5p, sme-miR-133b-3p, sme-miR-133b-5p, sme-miR-184-3p, sme-miR-184-5p, sme-miR-190a-3p, sme-miR-190a-5p, sme-miR-190b-3p, sme-miR-190b-5p, sme-miR-1992-3p, sme-miR-1992-5p, sme-miR-1993-3p, sme-miR-1993-5p, sme-miR-1a-3p, sme-miR-1a-5p, sme-miR-1b-3p, sme-miR-1b-5p, sme-miR-1c-3p, sme-miR-1c-5p, sme-miR-2147a, sme-miR-2147b, sme-miR-2147c, sme-miR-2147d-3p, sme-miR-2147d-5p, sme-miR-2148-3p, sme-miR-2148-5p, sme-miR-2149-3p, sme-miR-2149-5p, sme-miR-2150, sme-miR-2151-3p, sme-miR-2151-5p, sme-miR-2152, sme-miR-2153, sme-miR-2154-3p, sme-miR-2154-5p, sme-miR-2155, sme-miR-2156a-3p, sme-miR-2156a-5p, sme-miR-2156b-3p, sme-miR-2156b-5p, sme-miR-2157-3p, sme-miR-2157-5p, sme-miR-2158-3p, sme-miR-2158-5p, sme-miR-2159-3p, sme-miR-2159-5p, sme-miR-216-3p, sme-miR-216-5p, sme-miR-2160-1-3p, sme-miR-2160-2-3p, sme-miR-2160-5p, sme-miR-2161-3p, sme-miR-2161-5p, sme-miR-2162-3p, sme-miR-2162-5p, sme-miR-2163-3p, sme-miR-2163-5p, sme-miR-2164-3p, sme-miR-2164-5p, sme-miR-2165-3p, sme-miR-2165-5p, sme-miR-2166, sme-miR-2167, sme-miR-2168-3p, sme-miR-2168-5p, sme-miR-2169-3p, sme-miR-2169-5p, sme-miR-2170, sme-miR-2171, sme-miR-2172-3p, sme-miR-2172-5p, sme-miR-2173-3p, sme-miR-2173-5p, sme-miR-2174, sme-miR-2175, sme-miR-2176, sme-miR-2177-3p, sme-miR-2177-5p, sme-miR-2178, sme-miR-2179-3p, sme-miR-2179-5p, sme-miR-2180, sme-miR-2181, sme-miR-219-3p, sme-miR-219-5p, sme-miR-2200-3p, sme-miR-2200-5p, sme-miR-2201-3p, sme-miR-2201-5p, sme-miR-2202-3p, sme-miR-2202-5p, sme-miR-2203-3p, sme-miR-2203-5p, sme-miR-2204-3p, sme-miR-2204-5p, sme-miR-2205-3p, sme-miR-2205-5p, sme-miR-2206-3p, sme-miR-2206-5p, sme-miR-277a-3p, sme-miR-277a-5p, sme-miR-277b-3p, sme-miR-277b-5p, sme-miR-277c-3p, sme-miR-277c-5p, sme-miR-277d-3p, sme-miR-277d-5p, sme-miR-278-3p, sme-miR-278-5p, sme-miR-281-3p, sme-miR-281-5p, sme-miR-2a-1-5p, sme-miR-2a-2-5p, sme-miR-2a-3p, sme-miR-2b-3p, sme-miR-2b-5p, sme-miR-2c-3p, sme-miR-2c-5p, sme-miR-2d-3p, sme-miR-2d-5p, sme-miR-2e-3p, sme-miR-2e-5p, sme-miR-2f-3p, sme-miR-2f-5p, sme-miR-315-3p, sme-miR-315-5p, sme-miR-31a-3p, sme-miR-31a-5p, sme-miR-31b-1-3p, sme-miR-31b-3p, sme-miR-31b-5p, sme-miR-36a-3p, sme-miR-36a-5p, sme-miR-36b-3p, sme-miR-36b-5p, sme-miR-36c-3p, sme-miR-36c-5p, sme-miR-61a-3p, sme-miR-61a-5p, sme-miR-61b-3p, sme-miR-61b-5p, sme-miR-67-3p, sme-miR-67-5p, sme-miR-71a-1-3p, sme-miR-71a-2-3p, sme-miR-71a-5p, sme-miR-71b-3p, sme-miR-71b-5p, sme-miR-71c-3p, sme-miR-71c-5p, sme-miR-745-3p, sme-miR-745-5p, sme-miR-746-3p, sme-miR-746-5p, sme-miR-747-3p, sme-miR-747-5p, sme-miR-748-3p, sme-miR-748-5p, sme-miR-749, sme-miR-750-3p, sme-miR-750-5p, sme-miR-751, sme-miR-752-3p, sme-miR-752-5p, sme-miR-753a, sme-miR-753b-3p, sme-miR-753b-5p, sme-miR-753c, sme-miR-753d-3p, sme-miR-753d-5p, sme-miR-753e-3p, sme-miR-753e-5p, sme-miR-754a, sme-miR-754b-3p, sme-miR-754b-5p, sme-miR-754c-1-3p, sme-miR-754c-2-3p, sme-miR-754c-5p, sme-miR-754d, sme-miR-755-3p, sme-miR-755-5p, sme-miR-756-3p, sme-miR-756-5p, sme-miR-76-3p, sme-miR-76-5p, sme-miR-79-3p, sme-miR-79-5p, sme-miR-7a-3p, sme-miR-7a-5p, sme-miR-7b-3p, sme-miR-7b-5p, sme-miR-7c-3p, sme-miR-7c-5p, sme-miR-7d-3p, sme-miR-7d-5p, sme-miR-87a-3p, sme-miR-87a-5p, sme-miR-87b-3p, sme-miR-87b-5p, sme-miR-87c-3p, sme-miR-87c-5p, sme-miR-87d-3p, sme-miR-87d-5p, sme-miR-8a-3p, sme-miR-8a-5p, sme-miR-8b-3p, sme-miR-8b-5p, sme-miR-92-3p, sme-miR-92-5p, sme-miR-96a-3p, sme-miR-96a-5p, sme-miR-96b-3p, sme-miR-96b-5p, sme-miR-9a-3p, sme-miR-9a-5p, sme-miR-9b-3p, sme-miR-9b-5p, smi-miR12112, smo-miR1080, smo-miR1081, smo-miR1082a, smo-miR1082b, smo-miR1083, smo-miR1084, smo-miR1085-3p, smo-miR1085-5p, smo-miR1086, smo-miR1087, smo-miR1088-3p, smo-miR1088-5p, smo-miR1089, smo-miR1090, smo-miR1091, smo-miR1092, smo-miR1093, smo-miR1094a, smo-miR1094b, smo-miR1094c, smo-miR1095a, smo-miR1095b, smo-miR1096, smo-miR1097, smo-miR1098, smo-miR1099, smo-miR1100, smo-miR1101-3p, smo-miR1101-5p, smo-miR1102, smo-miR1103-3p, smo-miR1103-5p, smo-miR1104, smo-miR1105, smo-miR1106, smo-miR1107, smo-miR1108, smo-miR1109, smo-miR1110, smo-miR1111, smo-miR1112-3p, smo-miR1112-5p, smo-miR1113, smo-miR1114, smo-miR1115-3p, smo-miR1115-5p, smo-miR156a, smo-miR156b, smo-miR156c, smo-miR156d, smo-miR159, smo-miR160a, smo-miR160b, smo-miR166a, smo-miR166b, smo-miR166c, smo-miR171a, smo-miR171b, smo-miR171c, smo-miR171d, smo-miR319, smo-miR396, smo-miR408, smo-miR536, smr-miR-282, smr-miR-3930-3p, smr-miR-3930-5p, smr-miR-965, sof-miR156, sof-miR159a, sof-miR159b, sof-miR159c, sof-miR159d, sof-miR159e, sof-miR167a, sof-miR167b, sof-miR168a, sof-miR168b, sof-miR396, sof-miR408a, sof-miR408b, sof-miR408c, sof-miR408d, sof-miR408e, spu-let-7, spu-miR-1, spu-miR-10, spu-miR-124, spu-miR-125-3p, spu-miR-125-5p, spu-miR-133, spu-miR-137, spu-miR-153-3p, spu-miR-153-5p, spu-miR-182, spu-miR-183, spu-miR-184, spu-miR-200-3p, spu-miR-200-5p, spu-miR-2001, spu-miR-2002-3p, spu-miR-2002-5p, spu-miR-2003-3p, spu-miR-2003-5p, spu-miR-2004, spu-miR-2005, spu-miR-2006, spu-miR-2007, spu-miR-2008, spu-miR-2009, spu-miR-2010, spu-miR-2011, spu-miR-2012, spu-miR-2013, spu-miR-210, spu-miR-219-3p, spu-miR-219-5p, spu-miR-22, spu-miR-242, spu-miR-252a, spu-miR-252b, spu-miR-278-3p, spu-miR-278-5p, spu-miR-29a, spu-miR-29b, spu-miR-31, spu-miR-33, spu-miR-34, spu-miR-375, spu-miR-4847, spu-miR-4848a, spu-miR-4848b, spu-miR-4849, spu-miR-4850, spu-miR-4851, spu-miR-4852, spu-miR-4853, spu-miR-4854, spu-miR-4855, spu-miR-5992, spu-miR-5993, spu-miR-5994, spu-miR-7, spu-miR-71, spu-miR-9-3p, spu-miR-9-5p, spu-miR-92a, spu-miR-92b-3p, spu-miR-92b-5p, spu-miR-92c, spu-miR-92d, spu-miR-92e, spu-miR-96, spu-miR-981, ssa-let-7a-3-3p, ssa-let-7a-3p, ssa-let-7a-4-3p, ssa-let-7a-5-3p, ssa-let-7a-5p, ssa-let-7b-3p, ssa-let-7b-5p, ssa-let-7c-2-3p, ssa-let-7c-3p, ssa-let-7c-5p, ssa-let-7d-3p, ssa-let-7d-5p, ssa-let-7e-3p, ssa-let-7e-5p, ssa-let-7f-3p, ssa-let-7f-5p, ssa-let-7g-3p, ssa-let-7g-5p, ssa-let-7 h-3p, ssa-let-7 h-5p, ssa-let-7i-2-3p, ssa-let-7i-3p, ssa-let-7i-5p, ssa-let-7j-3p, ssa-let-7j-5p, ssa-miR-1-3-5p, ssa-miR-1-3p, ssa-miR-1-4-5p, ssa-miR-1-5p, ssa-miR-100a-2-3p, ssa-miR-100a-3p, ssa-miR-100a-5p, ssa-miR-101a-3p, ssa-miR-101a-5p, ssa-miR-101b-3p, ssa-miR-101b-5p, ssa-miR-103-3p, ssa-miR-103-5p, ssa-miR-106a-3p, ssa-miR-106a-5p, ssa-miR-106b-3p, ssa-miR-106b-5p, ssa-miR-107-3p, ssa-miR-107-5p, ssa-miR-10a-2-3p, ssa-miR-10a-3p, ssa-miR-10a-5p, ssa-miR-10b-2-3p, ssa-miR-10b-3-3p, ssa-miR-10b-3p, ssa-miR-10b-5p, ssa-miR-10d-3p, ssa-miR-10d-5p, ssa-miR-122-2-3p, ssa-miR-122-3p, ssa-miR-122-5p, ssa-miR-125a-2-3p, ssa-miR-125a-3p, ssa-miR-125a-5p, ssa-miR-125b-1-3p, ssa-miR-125b-2-3p, ssa-miR-125b-3-3p, ssa-miR-125b-5p, ssa-miR-126-3p, ssa-miR-126-5p, ssa-miR-128-1-5p, ssa-miR-128-2-5p, ssa-miR-128-3-5p, ssa-miR-128-3p, ssa-miR-128-4-5p, ssa-miR-129-3-3p, ssa-miR-129-3p, ssa-miR-129-5p, ssa-miR-130a-2-3p, ssa-miR-130a-3p, ssa-miR-130a-5p, ssa-miR-130b-2-5p, ssa-miR-130b-3p, ssa-miR-130b-5p, ssa-miR-130d-1-5p, ssa-miR-130d-2-5p, ssa-miR-130d-3-5p, ssa-miR-130d-3p, ssa-miR-132-3-5p, ssa-miR-132-3p, ssa-miR-132-4-5p, ssa-miR-132-5p, ssa-miR-1338-3p, ssa-miR-1338-5p, ssa-miR-133a-3-5p, ssa-miR-133a-3p, ssa-miR-133a-5p, ssa-miR-133b-3p, ssa-miR-133b-5p, ssa-miR-135a-3p, ssa-miR-135a-5p, ssa-miR-135b-1-3p, ssa-miR-135b-2-3p, ssa-miR-135b-3-3p, ssa-miR-135b-5p, ssa-miR-135c-3p, ssa-miR-135c-5p, ssa-miR-137-3p, ssa-miR-137-5p, ssa-miR-138-3-3p, ssa-miR-138-3p, ssa-miR-138-4-3p, ssa-miR-138-5p, ssa-miR-139-2-3p, ssa-miR-139-3p, ssa-miR-139-5p, ssa-miR-140-3p, ssa-miR-140-5p, ssa-miR-142a-3p, ssa-miR-142a-5p, ssa-miR-142b-3p, ssa-miR-142b-5p, ssa-miR-143-3p, ssa-miR-143-5p, ssa-miR-144-3p, ssa-miR-144-5p, ssa-miR-145-3p, ssa-miR-145-5p, ssa-miR-146a-3-3p, ssa-miR-146a-3p, ssa-miR-146a-5p, ssa-miR-146b-3p, ssa-miR-146b-5p, ssa-miR-146d-2-3p, ssa-miR-146d-3p, ssa-miR-146d-5p, ssa-miR-148a-3p, ssa-miR-148a-5p, ssa-miR-148b-3p, ssa-miR-148b-5p, ssa-miR-150-3p, ssa-miR-150-5p, ssa-miR-152-3p, ssa-miR-152-5p, ssa-miR-153a-2-5p, ssa-miR-153a-3p, ssa-miR-153a-5p, ssa-miR-153b-3p, ssa-miR-153b-5p, ssa-miR-155-3p, ssa-miR-155-5p, ssa-miR-15a-3p, ssa-miR-15a-5p, ssa-miR-15b-3p, ssa-miR-15b-5p, ssa-miR-15c-2-3p, ssa-miR-15c-3p, ssa-miR-15c-5p, ssa-miR-15d-3p, ssa-miR-15d-5p, ssa-miR-15e-3p, ssa-miR-15e-5p, ssa-miR-16a-2-3p, ssa-miR-16a-3p, ssa-miR-16a-5p, ssa-miR-16b-3p, ssa-miR-16b-5p, ssa-miR-16c-3p, ssa-miR-16c-5p, ssa-miR-17-3-3p, ssa-miR-17-3p, ssa-miR-17-5p, ssa-miR-1788-3p, ssa-miR-1788-5p, ssa-miR-181a-1-3p, ssa-miR-181a-2-3p, ssa-miR-181a-3-3p, ssa-miR-181a-4-3p, ssa-miR-181a-5-3p, ssa-miR-181a-5p, ssa-miR-181b-3p, ssa-miR-181b-5p, ssa-miR-181c-3p, ssa-miR-181c-5p, ssa-miR-182-3p, ssa-miR-182-5p, ssa-miR-183-2-3p, ssa-miR-183-3p, ssa-miR-183-5p, ssa-miR-18a-3p, ssa-miR-18a-5p, ssa-miR-18b-3p, ssa-miR-18b-5p, ssa-miR-190a-3p, ssa-miR-190a-5p, ssa-miR-190b-3p, ssa-miR-190b-5p, ssa-miR-192a-2-3p, ssa-miR-192a-3p, ssa-miR-192a-5p, ssa-miR-192b-3p, ssa-miR-192b-5p, ssa-miR-193-3p, ssa-miR-193-5p, ssa-miR-194a-3p, ssa-miR-194a-5p, ssa-miR-194b-3p, ssa-miR-194b-5p, ssa-miR-194c-3p, ssa-miR-194c-5p, ssa-miR-196a-1-3p, ssa-miR-196a-2-3p, ssa-miR-196a-3-3p, ssa-miR-196a-4-3p, ssa-miR-196a-5p, ssa-miR-196b-3p, ssa-miR-196b-5p, ssa-miR-199a-3p, ssa-miR-199a-5p, ssa-miR-19a-3-5p, ssa-miR-19a-3p, ssa-miR-19a-5p, ssa-miR-19c-3-5p, ssa-miR-19c-3p, ssa-miR-19c-4-5p, ssa-miR-19c-5p, ssa-miR-19d-3p, ssa-miR-19d-5p, ssa-miR-200a-1-5p, ssa-miR-200a-2-5p, ssa-miR-200a-3-5p, ssa-miR-200a-3p, ssa-miR-200b-3p, ssa-miR-200b-5p, ssa-miR-202-3p, ssa-miR-202-5p, ssa-miR-203a-2-5p, ssa-miR-203a-3p, ssa-miR-203a-5p, ssa-miR-203b-3p, ssa-miR-203b-5p, ssa-miR-204-3-3p, ssa-miR-204-3p, ssa-miR-204-4-3p, ssa-miR-204-5p, ssa-miR-205a-2-3p, ssa-miR-205a-3p, ssa-miR-205a-5p, ssa-miR-205b-1-3p, ssa-miR-205b-2-3p, ssa-miR-205b-3-3p, ssa-miR-205b-4-3p, ssa-miR-205b-5p, ssa-miR-206-3p, ssa-miR-206-5p, ssa-miR-20a-1-3p, ssa-miR-20a-2-3p, ssa-miR-20a-3-3p, ssa-miR-20a-5p, ssa-miR-20b-3p, ssa-miR-20b-5p, ssa-miR-210-3p, ssa-miR-210-5p, ssa-miR-212a-3p, ssa-miR-212a-5p, ssa-miR-212b-3p, ssa-miR-212b-5p, ssa-miR-214-3p, ssa-miR-214-5p, ssa-miR-216a-3p, ssa-miR-216a-5p, ssa-miR-216b-3p, ssa-miR-216b-5p, ssa-miR-218-3p, ssa-miR-218-5p, ssa-miR-2184-3p, ssa-miR-2184-5p, ssa-miR-2187-3p, ssa-miR-2187-5p, ssa-miR-2188-3p, ssa-miR-2188-5p, ssa-miR-219a-3p, ssa-miR-219a-5p, ssa-miR-219b-3p, ssa-miR-219b-5p, ssa-miR-219c-2-3p, ssa-miR-219c-3p, ssa-miR-219c-5p, ssa-miR-21a-2-3p, ssa-miR-21a-3p, ssa-miR-21a-5p, ssa-miR-21b-3p, ssa-miR-21b-5p, ssa-miR-221-3p, ssa-miR-221-5p, ssa-miR-222a-3p, ssa-miR-222a-5p, ssa-miR-222b-3p, ssa-miR-222b-5p, ssa-miR-22a-3p, ssa-miR-22a-5p, ssa-miR-22b-3p, ssa-miR-22b-5p, ssa-miR-23a-3-5p, ssa-miR-23a-3p, ssa-miR-23a-4-5p, ssa-miR-23a-5p, ssa-miR-23b-3p, ssa-miR-23b-5p, ssa-miR-24a-3-5p, ssa-miR-24a-3p, ssa-miR-24a-4-5p, ssa-miR-24a-5p, ssa-miR-24b-3p, ssa-miR-24b-5p, ssa-miR-25-3-5p, ssa-miR-25-3p, ssa-miR-25-5p, ssa-miR-26a-3-3p, ssa-miR-26a-3p, ssa-miR-26a-4-3p, ssa-miR-26a-5-3p, ssa-miR-26a-5p, ssa-miR-26a-6-3p, ssa-miR-26b-3p, ssa-miR-26b-5p, ssa-miR-26d-3p, ssa-miR-26d-5p, ssa-miR-27a-2-5p, ssa-miR-27a-3p, ssa-miR-27a-5p, ssa-miR-27b-3p, ssa-miR-27b-5p, ssa-miR-27c-2-5p, ssa-miR-27c-3p, ssa-miR-27c-5p, ssa-miR-27d-3p, ssa-miR-27d-5p, ssa-miR-29a-3p, ssa-miR-29a-5p, ssa-miR-29b-1-5p, ssa-miR-29b-2-5p, ssa-miR-29b-3-5p, ssa-miR-29b-3p, ssa-miR-29b-4-5p, ssa-miR-29c-3p, ssa-miR-29c-5p, ssa-miR-301a-3p, ssa-miR-301a-5p, ssa-miR-301b-3p, ssa-miR-301b-5p, ssa-miR-301c-3p, ssa-miR-301c-5p, ssa-miR-301d-3p, ssa-miR-301d-5p, ssa-miR-30a-3-3p, ssa-miR-30a-3p, ssa-miR-30a-4-3p, ssa-miR-30a-5p, ssa-miR-30b-3p, ssa-miR-30b-5p, ssa-miR-30c-3p, ssa-miR-30c-5p, ssa-miR-30d-2-3p, ssa-miR-30d-3p, ssa-miR-30d-5p, ssa-miR-30e-3-3p, ssa-miR-30e-3p, ssa-miR-30e-5p, ssa-miR-338a-3-5p, ssa-miR-338a-3p, ssa-miR-338a-4-5p, ssa-miR-338a-5p, ssa-miR-33a-3p, ssa-miR-33a-5p, ssa-miR-33b-3p, ssa-miR-33b-5p, ssa-miR-365-2-5p, ssa-miR-365-3p, ssa-miR-365-5p, ssa-miR-375-2-5p, ssa-miR-375-3-5p, ssa-miR-375-3p, ssa-miR-375-5p, ssa-miR-429-3p, ssa-miR-429-5p, ssa-miR-430a-3p, ssa-miR-430a-5p, ssa-miR-430b-3p, ssa-miR-430b-5p, ssa-miR-430c-3p, ssa-miR-430c-5p, ssa-miR-449a-3p, ssa-miR-449a-5p, ssa-miR-449b-3p, ssa-miR-449b-5p, ssa-miR-454-3p, ssa-miR-454-5p, ssa-miR-455-3p, ssa-miR-455-5p, ssa-miR-456-3p, ssa-miR-456-5p, ssa-miR-458-3p, ssa-miR-458-5p, ssa-miR-459-3p, ssa-miR-459-5p, ssa-miR-460-3p, ssa-miR-460-5p, ssa-miR-462a-3p, ssa-miR-462a-5p, ssa-miR-462b-3p, ssa-miR-462b-5p, ssa-miR-489-3p, ssa-miR-489-5p, ssa-miR-499a-3p, ssa-miR-499a-5p, ssa-miR-499b-3p, ssa-miR-499b-5p, ssa-miR-551-3p, ssa-miR-551-5p, ssa-miR-7132a-3p, ssa-miR-7132a-5p, ssa-miR-7132b-3p, ssa-miR-7132b-5p, ssa-miR-722-3p, ssa-miR-722-5p, ssa-miR-723-3p, ssa-miR-723-5p, ssa-miR-724-3p, ssa-miR-724-5p, ssa-miR-725-3p, ssa-miR-725-5p, ssa-miR-727-3p, ssa-miR-727-5p, ssa-miR-729-3p, ssa-miR-729-5p, ssa-miR-730a-2-3p, ssa-miR-730a-3p, ssa-miR-730a-5p, ssa-miR-731-3p, ssa-miR-731-5p, ssa-miR-734-3p, ssa-miR-734-5p, ssa-miR-736-3p, ssa-miR-736-5p, ssa-miR-737-3p, ssa-miR-737-5p, ssa-miR-7552a-2-3p, ssa-miR-7552a-3p, ssa-miR-7552a-5p, ssa-miR-7552b-3p, ssa-miR-7552b-5p, ssa-miR-7a-1-3p, ssa-miR-7a-2-3p, ssa-miR-7a-3-3p, ssa-miR-7a-4-3p, ssa-miR-7a-5-3p, ssa-miR-7a-5p, ssa-miR-7a-6-3p, ssa-miR-7a-7-3p, ssa-miR-8156-3p, ssa-miR-8156-5p, ssa-miR-8157-3p, ssa-miR-8157-5p, ssa-miR-8158-3p, ssa-miR-8158-5p, ssa-miR-8159-3p, ssa-miR-8159-5p, ssa-miR-8160-2-3p, ssa-miR-8160-3p, ssa-miR-8160-5p, ssa-miR-8162-3p, ssa-miR-8162-5p, ssa-miR-8163-3p, ssa-miR-8163-5p, ssa-miR-8164-3p, ssa-miR-8164-5p, ssa-miR-92a-3-5p, ssa-miR-92a-3p, ssa-miR-92a-4-5p, ssa-miR-92a-5p, ssa-miR-92b-3p, ssa-miR-92b-5p, ssa-miR-93a-2-3p, ssa-miR-93a-3p, ssa-miR-93a-5p, ssa-miR-96-3p, ssa-miR-96-5p, ssa-miR-99-2-3p, ssa-miR-99-3p, ssa-miR-99-5p, ssa-miR-9a-1-3p, ssa-miR-9a-2-3p, ssa-miR-9a-3-3p, ssa-miR-9a-4-3p, ssa-miR-9a-5-3p, ssa-miR-9a-5p, ssa-miR-9a-6-3p, ssa-miR-9a-7-3p, ssa-miR-9a-8-3p, ssa-miR-9b-3p, ssa-miR-9b-5p, ssc-let-7a, ssc-let-7c, ssc-let-7d-3p, ssc-let-7d-5p, ssc-let-7e, ssc-let-7f-3p, ssc-let-7f-5p, ssc-let-7g, ssc-let-7i-3p, ssc-let-7i-5p, ssc-miR-1, ssc-miR-100, ssc-miR-101, ssc-miR-103, ssc-miR-10382, ssc-miR-10383, ssc-miR-10384, ssc-miR-10386, ssc-miR-10387, ssc-miR-10388, ssc-miR-10389, ssc-miR-10390, ssc-miR-10391, ssc-miR-105-1, ssc-miR-105-2, ssc-miR-106a, ssc-miR-107, ssc-miR-10a-3p, ssc-miR-10a-5p, ssc-miR-10b, ssc-miR-122-3p, ssc-miR-122-5p, ssc-miR-1224, ssc-miR-1249, ssc-miR-124a, ssc-miR-125a, ssc-miR-125b, ssc-miR-126-3p, ssc-miR-126-5p, ssc-miR-127, ssc-miR-1271-3p, ssc-miR-1271-5p, ssc-miR-1277, ssc-miR-128, ssc-miR-1285, ssc-miR-1296-3p, ssc-miR-1296-5p, ssc-miR-129a-3p, ssc-miR-129a-5p, ssc-miR-129b, ssc-miR-1306-3p, ssc-miR-1306-5p, ssc-miR-1307, ssc-miR-130a, ssc-miR-130b-3p, ssc-miR-130b-5p, ssc-miR-132, ssc-miR-133a-3p, ssc-miR-133a-5p, ssc-miR-133b, ssc-miR-1343, ssc-miR-135, ssc-miR-136-3p, ssc-miR-136-5p, ssc-miR-137, ssc-miR-138, ssc-miR-1388, ssc-miR-139-3p, ssc-miR-139-5p, ssc-miR-140-3p, ssc-miR-140-5p, ssc-miR-141, ssc-miR-142-3p, ssc-miR-142-5p, ssc-miR-143-3p, ssc-miR-143-5p, ssc-miR-144, ssc-miR-145-3p, ssc-miR-145-5p, ssc-miR-1468, ssc-miR-146a-3p, ssc-miR-146a-5p, ssc-miR-146b, ssc-miR-148a-3p, ssc-miR-148a-5p, ssc-miR-148b-3p, ssc-miR-148b-5p, ssc-miR-149, ssc-miR-150, ssc-miR-151-3p, ssc-miR-151-5p, ssc-miR-152, ssc-miR-153, ssc-miR-155-3p, ssc-miR-155-5p, ssc-miR-15a, ssc-miR-15b, ssc-miR-16, ssc-miR-17-3p, ssc-miR-17-5p, ssc-miR-181a, ssc-miR-181b, ssc-miR-181c, ssc-miR-181d-3p, ssc-miR-181d-5p, ssc-miR-182, ssc-miR-183, ssc-miR-1839-3p, ssc-miR-1839-5p, ssc-miR-184, ssc-miR-1842, ssc-miR-185, ssc-miR-186-3p, ssc-miR-186-5p, ssc-miR-187, ssc-miR-18a, ssc-miR-18b, ssc-miR-190a, ssc-miR-190b, ssc-miR-191, ssc-miR-192, ssc-miR-193a-3p, ssc-miR-193a-5p, ssc-miR-194a-3p, ssc-miR-194a-5p, ssc-miR-194b-3p, ssc-miR-194b-5p, ssc-miR-195, ssc-miR-196a, ssc-miR-196b-3p, ssc-miR-196b-5p, ssc-miR-199a-3p, ssc-miR-199a-5p, ssc-miR-199b-3p, ssc-miR-199b-5p, ssc-miR-19a, ssc-miR-19b, ssc-miR-200b, ssc-miR-202-3p, ssc-miR-202-5p, ssc-miR-204, ssc-miR-205, ssc-miR-206, ssc-miR-208b, ssc-miR-20a-3p, ssc-miR-20a-5p, ssc-miR-20b, ssc-miR-21-3p, ssc-miR-21-5p, ssc-miR-210, ssc-miR-212, ssc-miR-214-3p, ssc-miR-214-5p, ssc-miR-215, ssc-miR-216, ssc-miR-217, ssc-miR-218, ssc-miR-218-3p, ssc-miR-218-5p, ssc-miR-218b, ssc-miR-219a, ssc-miR-219b-3p, ssc-miR-22-3p, ssc-miR-22-5p, ssc-miR-221-3p, ssc-miR-221-5p, ssc-miR-222, ssc-miR-223, ssc-miR-224, ssc-miR-2320-3p, ssc-miR-2320-5p, ssc-miR-2366, ssc-miR-23a, ssc-miR-23b, ssc-miR-24-1-5p, ssc-miR-24-2-5p, ssc-miR-24-3p, ssc-miR-2411, ssc-miR-2483, ssc-miR-26a, ssc-miR-26b-3p, ssc-miR-26b-5p, ssc-miR-27a, ssc-miR-27b-3p, ssc-miR-27b-5p, ssc-miR-28-3p, ssc-miR-28-5p, ssc-miR-296-3p, ssc-miR-296-5p, ssc-miR-299, ssc-miR-29a-3p, ssc-miR-29a-5p, ssc-miR-29b, ssc-miR-29c, ssc-miR-301, ssc-miR-30a-3p, ssc-miR-30a-5p, ssc-miR-30b-3p, ssc-miR-30b-5p, ssc-miR-30c-1-3p, ssc-miR-30c-3p, ssc-miR-30c-5p, ssc-miR-30d, ssc-miR-30e-3p, ssc-miR-30e-5p, ssc-miR-31, ssc-miR-32, ssc-miR-320, ssc-miR-323, ssc-miR-324, ssc-miR-325, ssc-miR-326, ssc-miR-328, ssc-miR-330, ssc-miR-331-3p, ssc-miR-331-5p, ssc-miR-335, ssc-miR-338, ssc-miR-339, ssc-miR-339-3p, ssc-miR-339-5p, ssc-miR-33b-3p, ssc-miR-33b-5p, ssc-miR-340, ssc-miR-342, ssc-miR-345-3p, ssc-miR-345-5p, ssc-miR-34a, ssc-miR-34c, ssc-miR-361-3p, ssc-miR-361-5p, ssc-miR-3613, ssc-miR-362, ssc-miR-363, ssc-miR-365-3p, ssc-miR-365-5p, ssc-miR-369, ssc-miR-370, ssc-miR-371-5p, ssc-miR-374a-3p, ssc-miR-374a-5p, ssc-miR-374b-3p, ssc-miR-374b-5p, ssc-miR-375, ssc-miR-376a-3p, ssc-miR-376a-5p, ssc-miR-376b, ssc-miR-376c, ssc-miR-378, ssc-miR-378b-3p, ssc-miR-381-3p, ssc-miR-381-5p, ssc-miR-382, ssc-miR-383, ssc-miR-411, ssc-miR-421-3p, ssc-miR-421-5p, ssc-miR-423-3p, ssc-miR-423-5p, ssc-miR-424-3p, ssc-miR-424-5p, ssc-miR-425-3p, ssc-miR-425-5p, ssc-miR-429, ssc-miR-432-3p, ssc-miR-432-5p, ssc-miR-4331-3p, ssc-miR-4331-5p, ssc-miR-4332, ssc-miR-4334-3p, ssc-miR-4334-5p, ssc-miR-4335, ssc-miR-4337, ssc-miR-4338, ssc-miR-4339, ssc-miR-450a, ssc-miR-450b-3p, ssc-miR-450b-5p, ssc-miR-450c-3p, ssc-miR-450c-5p, ssc-miR-451, ssc-miR-452, ssc-miR-454, ssc-miR-455-3p, ssc-miR-455-5p, ssc-miR-484, ssc-miR-486, ssc-miR-487b, ssc-miR-489, ssc-miR-490, ssc-miR-490-3p, ssc-miR-490-5p, ssc-miR-491, ssc-miR-493-3p, ssc-miR-493-5p, ssc-miR-494, ssc-miR-497, ssc-miR-499-3p, ssc-miR-499-5p, ssc-miR-500-3p, ssc-miR-500-5p, ssc-miR-503, ssc-miR-504, ssc-miR-505, ssc-miR-532-3p, ssc-miR-532-5p, ssc-miR-542-3p, ssc-miR-542-5p, ssc-miR-545-3p, ssc-miR-545-5p, ssc-miR-551a, ssc-miR-574-3p, ssc-miR-574-5p, ssc-miR-582-3p, ssc-miR-582-5p, ssc-miR-615, ssc-miR-628-3p, ssc-miR-628-5p, ssc-miR-6516, ssc-miR-652, ssc-miR-6529, ssc-miR-660, ssc-miR-664-3p, ssc-miR-664-5p, ssc-miR-670, ssc-miR-671-3p, ssc-miR-671-5p, ssc-miR-676-3p, ssc-miR-676-5p, ssc-miR-6782-3p, ssc-miR-7-3p, ssc-miR-7-5p, ssc-miR-708-3p, ssc-miR-708-5p, ssc-miR-7134-3p, ssc-miR-7134-5p, ssc-miR-7135-3p, ssc-miR-7135-5p, ssc-miR-7136-3p, ssc-miR-7136-5p, ssc-miR-7137-3p, ssc-miR-7137-5p, ssc-miR-7138-3p, ssc-miR-7138-5p, ssc-miR-7139-3p, ssc-miR-7139-5p, ssc-miR-7140-3p, ssc-miR-7140-5p, ssc-miR-7141-3p, ssc-miR-7141-5p, ssc-miR-7142-3p, ssc-miR-7142-5p, ssc-miR-7143-3p, ssc-miR-7143-5p, ssc-miR-7144-3p, ssc-miR-7144-5p, ssc-miR-744, ssc-miR-758, ssc-miR-769-3p, ssc-miR-769-5p, ssc-miR-7857-3p, ssc-miR-802, ssc-miR-874, ssc-miR-885-3p, ssc-miR-885-5p, ssc-miR-9, ssc-miR-9-1, ssc-miR-9-2, ssc-miR-92a, ssc-miR-92b-3p, ssc-miR-92b-5p, ssc-miR-935, ssc-miR-95, ssc-miR-96-5p, ssc-miR-9784-5p, ssc-miR-9785-5p, ssc-miR-9786-3p, ssc-miR-9787-3p, ssc-miR-9788-3p, ssc-miR-9789-3p, ssc-miR-9790-3p, ssc-miR-9791-3p, ssc-miR-9792-5p, ssc-miR-9793-5p, ssc-miR-9794-3p, ssc-miR-9795-3p, ssc-miR-9796-3p, ssc-miR-9797-3p, ssc-miR-9798-3p, ssc-miR-9799-3p, ssc-miR-98, ssc-miR-9800-3p, ssc-miR-9801-5p, ssc-miR-9802-3p, ssc-miR-9803-5p, ssc-miR-9804-5p, ssc-miR-9805-3p, ssc-miR-9806-5p, ssc-miR-9807-5p, ssc-miR-9808-3p, ssc-miR-9809-3p, ssc-miR-9810-3p, ssc-miR-9811-5p, ssc-miR-9812-3p, ssc-miR-9813-5p, ssc-miR-9814-3p, ssc-miR-9815-3p, ssc-miR-9816-3p, ssc-miR-9817-5p, ssc-miR-9818-3p, ssc-miR-9819-5p, ssc-miR-9820-5p, ssc-miR-9821-5p, ssc-miR-9822-3p, ssc-miR-9823-5p, ssc-miR-9824-5p, ssc-miR-9825-5p, ssc-miR-9826-5p, ssc-miR-9827-5p, ssc-miR-9828-3p, ssc-miR-9829-5p, ssc-miR-9830-5p, ssc-miR-9831-3p, ssc-miR-9832-3p, ssc-miR-9833-5p, ssc-miR-9834-5p, ssc-miR-9835-3p, ssc-miR-9836-3p, ssc-miR-9837-5p, ssc-miR-9838-5p, ssc-miR-9839-5p, ssc-miR-9840-3p, ssc-miR-9841-3p, ssc-miR-9842-5p, ssc-miR-9843-3p, ssc-miR-9844-3p, ssc-miR-9845-5p, ssc-miR-9846-3p, ssc-miR-9847-3p, ssc-miR-9848-3p, ssc-miR-9849-5p, ssc-miR-9850-5p, ssc-miR-9851-3p, ssc-miR-9852-3p, ssc-miR-9853-5p, ssc-miR-9854-5p, ssc-miR-9855-5p, ssc-miR-9856-3p, ssc-miR-9857-5p, ssc-miR-9858-5p, ssc-miR-9859-3p, ssc-miR-9860-5p, ssc-miR-9861-5p, ssc-miR-9862-3p, ssc-miR-99a-3p, ssc-miR-99a-5p, ssc-miR-99b, ssl-miR1078, ssl-miR156, ssl-miR164a, ssl-miR164b, ssl-miR166a, ssl-miR166b, ssl-miR169, ssl-miR171a, ssl-miR171b, ssl-miR172, ssl-miR394, ssl-miR395, ssl-miR396, ssl-miR397, ssl-miR398, ssl-miR399, ssl-miR828, ssl-miR948, ssp-miR1128, ssp-miR1432, ssp-miR156, ssp-miR159a, ssp-miR166, ssp-miR167b, ssp-miR168a, ssp-miR169, ssp-miR396, ssp-miR408a, ssp-miR408d, ssp-miR437a, ssp-miR437b, ssp-miR437c, ssp-miR444a, ssp-miR444b.1, ssp-miR444b.2, ssp-miR444c-3p, ssp-miR528, ssp-miR827, ssy-miR-506, ssy-miR-507, ssy-miR-508, ssy-miR-509a, ssy-miR-509b, ssy-miR-510, ssy-miR-513a, ssy-miR-513b, ssy-miR-513c, ssy-miR-514, str-bantam-3p, str-bantam-5p, str-lin-4-3p, str-lin-4-5p, str-miR-1-3p, str-miR-1-5p, str-miR-1175-3p, str-miR-1175-5p, str-miR-124-3p, str-miR-124-5p, str-miR-184-3p, str-miR-184-5p, str-miR-2-3p, str-miR-2-5p, str-miR-234-3p, str-miR-234-5p, str-miR-236-3p, str-miR-236-5p, str-miR-240-3p, str-miR-240-5p, str-miR-252a-3p, str-miR-252a-5p, str-miR-252b-3p, str-miR-252b-5p, str-miR-255-3p, str-miR-255-5p, str-miR-279-3p, str-miR-279-5p, str-miR-34a-3p, str-miR-34a-5p, str-miR-34b-3p, str-miR-34b-5p, str-miR-34c-3p, str-miR-34c-5p, str-miR-360-3p, str-miR-360-5p, str-miR-37a-3p, str-miR-37a-5p, str-miR-37b-3p, str-miR-37b-5p, str-miR-40-3p, str-miR-40-5p, str-miR-47-3p, str-miR-47-5p, str-miR-50-3p, str-miR-50-5p, str-miR-5359-3p, str-miR-5359-5p, str-miR-58-3p, str-miR-58-5p, str-miR-71-3p, str-miR-71-5p, str-miR-72-3p, str-miR-72-5p, str-miR-76-3p, str-miR-76-5p, str-miR-7880a-3p, str-miR-7880a-5p, str-miR-7880b-1-5p, str-miR-7880b-2-5p, str-miR-7880b-3-5p, str-miR-7880b-3p, str-miR-7880b-4-5p, str-miR-7880c-3p, str-miR-7880c-5p, str-miR-7880d-3p, str-miR-7880d-5p, str-miR-7880e-2-5p, str-miR-7880e-3p, str-miR-7880e-5p, str-miR-7880f-3p, str-miR-7880f-5p, str-miR-7880g-3p, str-miR-7880g-5p, str-miR-7880 h-3p, str-miR-7880 h-5p, str-miR-7880i-3p, str-miR-7880i-5p, str-miR-7880j-3p, str-miR-7880j-5p, str-miR-7880k-3p, str-miR-7880k-5p, str-miR-78801-3p, str-miR-78801-5p, str-miR-7880m-3p, str-miR-7880m-5p, str-miR-7880n-3p, str-miR-7880n-5p, str-miR-78800-3p, str-miR-78800-5p, str-miR-7880p-3p, str-miR-7880p-5p, str-miR-7880q-3p, str-miR-7880q-5p, str-miR-7880r-3p, str-miR-7880r-5p, str-miR-7880s-3p, str-miR-7880s-5p, str-miR-7880t-3p, str-miR-7880t-5p, str-miR-7880u-3p, str-miR-7880u-5p, str-miR-7880v-3p, str-miR-7880v-5p, str-miR-7880w-3p, str-miR-7880w-5p, str-miR-7880x-3p, str-miR-7880x-5p, str-miR-7880y-3p, str-miR-7880y-5p, str-miR-81a-3p, str-miR-81a-5p, str-miR-81b-3p, str-miR-81b-5p, str-miR-81c-3p, str-miR-81c-5p, str-miR-8365-3p, str-miR-8365-5p, str-miR-8366a-3p, str-miR-8366a-5p, str-miR-8366b-3p, str-miR-8366b-5p, str-miR-8367-3p, str-miR-8367-5p, str-miR-8368-3p, str-miR-8368-5p, str-miR-8369-3p, str-miR-8369-5p, str-miR-8370-3p, str-miR-8370-5p, str-miR-8371-3p, str-miR-8371-5p, str-miR-8372-3p, str-miR-8372-5p, str-miR-8373-3p, str-miR-8373-5p, str-miR-8374-3p, str-miR-8374-5p, str-miR-8375-3p, str-miR-8375-5p, str-miR-8376-3p, str-miR-8376-5p, str-miR-8377-3p, str-miR-8377-5p, str-miR-8378-3p, str-miR-8378-5p, str-miR-8379a-3p, str-miR-8379a-5p, str-miR-8379b-3p, str-miR-8379b-5p, str-miR-8380-3p, str-miR-8380-5p, str-miR-8381-3p, str-miR-8381-5p, str-miR-8382-3p, str-miR-8382-5p, str-miR-8383-3p, str-miR-8383-5p, str-miR-8384-3p, str-miR-8384-5p, str-miR-8385-3p, str-miR-8385-5p, str-miR-8386-3p, str-miR-8386-5p, str-miR-8387-3p, str-miR-8387-5p, str-miR-8388-3p, str-miR-8388-5p, str-miR-8389-3p, str-miR-8389-5p, str-miR-8390-3p, str-miR-8390-5p, str-miR-8391-3p, str-miR-8391-5p, str-miR-8392-3p, str-miR-8392-5p, str-miR-8393-3p, str-miR-8393-5p, str-miR-8394-3p, str-miR-8394-5p, str-miR-8395-3p, str-miR-8395-5p, str-miR-8396-3p, str-miR-8396-5p, str-miR-8397-3p, str-miR-8397-5p, str-miR-8398-3p, str-miR-8398-5p, str-miR-8399-3p, str-miR-8399-5p, str-miR-84-3p, str-miR-84-5p, str-miR-8400-3p, str-miR-8400-5p, str-miR-8401-3p, str-miR-8401-5p, str-miR-8402-3p, str-miR-8402-5p, str-miR-86-3p, str-miR-86-5p, str-miR-87a-3p, str-miR-87a-5p, str-miR-87b-3p, str-miR-87b-5p, str-miR-9-3p, str-miR-9-5p, str-miR-92-3p, str-miR-92-5p, stu-miR156a, stu-miR156b, stu-miR156c, stu-miR156d-3p, stu-miR156d-5p, stu-miR156e, stu-miR156f-3p, stu-miR156f-5p, stu-miR156g-3p, stu-miR156g-5p, stu-miR156 h-3p, stu-miR156 h-5p, stu-miR156i-3p, stu-miR156i-5p, stu-miR156j-3p, stu-miR156j-5p, stu-miR156k-3p, stu-miR156k-5p, stu-miR160a-3p, stu-miR160a-5p, stu-miR160b, stu-miR162a-3p, stu-miR162a-5p, stu-miR162b-3p, stu-miR162b-5p, stu-miR164-3p, stu-miR164-5p, stu-miR166a-3p, stu-miR166a-5p, stu-miR166b, stu-miR166c-3p, stu-miR166c-5p, stu-miR166d-3p, stu-miR166d-5p, stu-miR167a-3p, stu-miR167a-5p, stu-miR167b-3p, stu-miR167b-5p, stu-miR167c-3p, stu-miR167c-5p, stu-miR167d-3p, stu-miR167d-5p, stu-miR169a-3p, stu-miR169a-5p, stu-miR169b-3p, stu-miR169b-5p, stu-miR169c-3p, stu-miR169c-5p, stu-miR169d-3p, stu-miR169d-5p, stu-miR169e-3p, stu-miR169e-5p, stu-miR169f-3p, stu-miR169f-5p, stu-miR169g, stu-miR169h, stu-miR171a-3p, stu-miR171a-5p, stu-miR171b-3p, stu-miR171b-5p, stu-miR171c-3p, stu-miR171c-5p, stu-miR171d-3p, stu-miR171d-5p, stu-miR171e, stu-miR172a-3p, stu-miR172a-5p, stu-miR172b-3p, stu-miR172b-5p, stu-miR172c-3p, stu-miR172c-5p, stu-miR172d-3p, stu-miR172d-5p, stu-miR172e-3p, stu-miR172e-5p, stu-miR1886a, stu-miR1886b, stu-miR1886c, stu-miR1886d, stu-miR1886e, stu-miR1886f, stu-miR1886g-3p, stu-miR1886g-5p, stu-miR1886h, stu-miR1886i-3p, stu-miR1886i-5p, stu-miR1919-3p, stu-miR1919-5p, stu-miR319-3p, stu-miR319-5p, stu-miR319a-3p, stu-miR319a-5p, stu-miR319b, stu-miR3627-3p, stu-miR3627-5p, stu-miR384-3p, stu-miR384-5p, stu-miR390-3p, stu-miR390-5p, stu-miR391-3p, stu-miR391-5p, stu-miR393-3p, stu-miR393-5p, stu-miR395a, stu-miR395b, stu-miR395c, stu-miR395d, stu-miR395e, stu-miR395f, stu-miR395g, stu-miR395h, stu-miR395i, stu-miR395j, stu-miR396-3p, stu-miR396-5p, stu-miR397-3p, stu-miR397-5p, stu-miR398a-3p, stu-miR398a-5p, stu-miR398b-3p, stu-miR398b-5p, stu-miR399a-3p, stu-miR399a-5p, stu-miR399b-3p, stu-miR399b-5p, stu-miR399c-3p, stu-miR399c-5p, stu-miR399d-3p, stu-miR399d-5p, stu-miR399e-3p, stu-miR399e-5p, stu-miR399f-3p, stu-miR399f-5p, stu-miR399g-3p, stu-miR399g-5p, stu-miR399h, stu-miR399i-3p, stu-miR399i-5p, stu-miR399j-3p, stu-miR399j-5p, stu-miR399k-3p, stu-miR399k-5p, stu-miR3991-3p, stu-miR3991-5p, stu-miR399m-3p, stu-miR399m-5p, stu-miR399n-3p, stu-miR399n-5p, stu-miR3990-3p, stu-miR3990-5p, stu-miR408a-3p, stu-miR408a-5p, stu-miR408b-3p, stu-miR408b-5p, stu-miR477a-3p, stu-miR477a-5p, stu-miR477b-3p, stu-miR477b-5p, stu-miR479, stu-miR482a-3p, stu-miR482a-5p, stu-miR482b-3p, stu-miR482b-5p, stu-miR482c, stu-miR482d-3p, stu-miR482d-5p, stu-miR482e-3p, stu-miR482e-5p, stu-miR530, stu-miR5303a, stu-miR5303b, stu-miR5303c, stu-miR5303d, stu-miR5303e, stu-miR5303f, stu-miR5303g, stu-miR5303h, stu-miR5303i, stu-miR5303j, stu-miR5304-3p, stu-miR5304-5p, stu-miR6022, stu-miR6023, stu-miR6024-3p, stu-miR6024-5p, stu-miR6025, stu-miR6026-3p, stu-miR6026-5p, stu-miR6027, stu-miR6149-3p, stu-miR6149-5p, stu-miR7122-3p, stu-miR7122-5p, stu-miR7979, stu-miR7980a, stu-miR7980b-3p, stu-miR7980b-5p, stu-miR7981-3p, stu-miR7981-5p, stu-miR7982a, stu-miR7982b, stu-miR7983-3p, stu-miR7983-5p, stu-miR7984a, stu-miR7984b-3p, stu-miR7984b-5p, stu-miR7984c-3p, stu-miR7984c-5p, stu-miR7984d-3p, stu-miR7984d-5p, stu-miR7985, stu-miR7986, stu-miR7987, stu-miR7988, stu-miR7989, stu-miR7990a, stu-miR7990b, stu-miR7991a, stu-miR7991b, stu-miR7991c, stu-miR7992-3p, stu-miR7992-5p, stu-miR7993a, stu-miR7993b-3p, stu-miR7993b-5p, stu-miR7993c, stu-miR7993d, stu-miR7994a, stu-miR7994b-3p, stu-miR7994b-5p, stu-miR7995, stu-miR7996a, stu-miR7996b, stu-miR7996c, stu-miR7997a, stu-miR7997b, stu-miR7997c, stu-miR7998, stu-miR7999-3p, stu-miR7999-5p, stu-miR8000, stu-miR8001a, stu-miR8001b-3p, stu-miR8001b-5p, stu-miR8002-3p, stu-miR8002-5p, stu-miR8003, stu-miR8004, stu-miR8005a, stu-miR8005b-3p, stu-miR8005b-5p, stu-miR8005c, stu-miR8006-3p, stu-miR8006-5p, stu-miR8007a-3p, stu-miR8007a-5p, stu-miR8007b-3p, stu-miR8007b-5p, stu-miR8008a, stu-miR8008b, stu-miR8009, stu-miR8010, stu-miR8011a-3p, stu-miR8011a-5p, stu-miR8011b-3p, stu-miR8011b-5p, stu-miR8012, stu-miR8013, stu-miR8014-3p, stu-miR8014-5p, stu-miR8015-3p, stu-miR8015-5p, stu-miR8016, stu-miR8017, stu-miR8018, stu-miR8019-3p, stu-miR8019-5p, stu-miR8020, stu-miR8021, stu-miR8022, stu-miR8023, stu-miR8024a-3p, stu-miR8024a-5p, stu-miR8024b, stu-miR8025-3p, stu-miR8025-5p, stu-miR8026, stu-miR8027, stu-miR8028-3p, stu-miR8028-5p, stu-miR8029, stu-miR8030-3p, stu-miR8030-5p, stu-miR8031, stu-miR8032a-3p, stu-miR8032a-5p, stu-miR8032b-3p, stu-miR8032b-5p, stu-miR8032c, stu-miR8032d-3p, stu-miR8032d-5p, stu-miR8032e-3p, stu-miR8032e-5p, stu-miR8032f-3p, stu-miR8032f-5p, stu-miR8032g-3p, stu-miR8032g-5p, stu-miR8033-3p, stu-miR8033-5p, stu-miR8034, stu-miR8035, stu-miR8036-3p, stu-miR8036-5p, stu-miR8037, stu-miR8038a-3p, stu-miR8038a-5p, stu-miR8038b-3p, stu-miR8038b-5p, stu-miR8039, stu-miR8040-3p, stu-miR8040-5p, stu-miR8041a-3p, stu-miR8041a-5p, stu-miR8041b-3p, stu-miR8041b-5p, stu-miR8042, stu-miR8043, stu-miR8044-3p, stu-miR8044-5p, stu-miR8045, stu-miR8046-3p, stu-miR8046-5p, stu-miR8047, stu-miR8048-3p, stu-miR8048-5p, stu-miR8049-3p, stu-miR8049-5p, stu-miR8050-3p, stu-miR8050-5p, stu-miR8051-3p, stu-miR8051-5p, stu-miR827-3p, stu-miR827-5p, sv40-miR-S1-3p, sv40-miR-S1-5p, tae-miR10516, tae-miR10517, tae-miR10518, tae-miR10519, tae-miR10520, tae-miR10521, tae-miR1117, tae-miR1118, tae-miR1119, tae-miR1120a, tae-miR1120b-3p, tae-miR1120c-5p, tae-miR1121, tae-miR1122a, tae-miR1122b-3p, tae-miR1122c-3p, tae-miR1123, tae-miR1124, tae-miR1125, tae-miR1127a, tae-miR1127b-3p, tae-miR1128, tae-miR1129, tae-miR1130a, tae-miR1130b-3p, tae-miR1131, tae-miR1133, tae-miR1134, tae-miR1135, tae-miR1136, tae-miR1137a, tae-miR1137b-5p, tae-miR1138, tae-miR1139, tae-miR156, tae-miR159a, tae-miR159b, tae-miR160, tae-miR164, tae-miR167a, tae-miR167b, tae-miR167c-5p, tae-miR169, tae-miR171a, tae-miR171b, tae-miR1847-5p, tae-miR2275-3p, tae-miR319, tae-miR395a, tae-miR395b, tae-miR396-5p, tae-miR397-5p, tae-miR398, tae-miR399, tae-miR408, tae-miR444a, tae-miR444b, tae-miR5048-5p, tae-miR5049-3p, tae-miR5050, tae-miR5062-5p, tae-miR5084, tae-miR5085, tae-miR5086, tae-miR5175-5p, tae-miR5200, tae-miR530, tae-miR531, tae-miR5384-3p, tae-miR6197-5p, tae-miR6201, tae-miR7757-5p, tae-miR9652-3p, tae-miR9652-5p, tae-miR9653a-3p, tae-miR9653b, tae-miR9654a-3p, tae-miR9654b-3p, tae-miR9655-3p, tae-miR9656-3p, tae-miR9657a-3p, tae-miR9657b-3p, tae-miR9657b-5p, tae-miR9657c-3p, tae-miR9658-3p, tae-miR9659-3p, tae-miR9660-5p, tae-miR9661-5p, tae-miR9662a-3p, tae-miR9662b-3p, tae-miR9663-5p, tae-miR9664-3p, tae-miR9665-3p, tae-miR9666a-3p, tae-miR9666b-3p, tae-miR9666b-5p, tae-miR9666c-5p, tae-miR9667-5p, tae-miR9668-5p, tae-miR9669-5p, tae-miR9670-3p, tae-miR9671-5p, tae-miR9672a-3p, tae-miR9672b, tae-miR9673-5p, tae-miR9674a-5p, tae-miR9674b-5p, tae-miR9675-3p, tae-miR9676-5p, tae-miR9677a, tae-miR9677b, tae-miR9678-3p, tae-miR9679-5p, tae-miR9772, tae-miR9773, tae-miR9774, tae-miR9775, tae-miR9776, tae-miR9777, tae-miR9778, tae-miR9779, tae-miR9780, tae-miR9781, tae-miR9782, tae-miR9783, tca-bantam-3p, tca-bantam-5p, tca-let-7-3p, tca-let-7-5p, tca-miR-1-3p, tca-miR-1-5p, tca-miR-10-3p, tca-miR-10-5p, tca-miR-100-3p, tca-miR-100-5p, tca-miR-1000-3p, tca-miR-1000-5p, tca-miR-11-3p, tca-miR-11-5p, tca-miR-11614-3p, tca-miR-11614-5p, tca-miR-11615-3p, tca-miR-11615-5p, tca-miR-11617a-3-3p, tca-miR-11617a-3p, tca-miR-11617a-4-3p, tca-miR-11617a-5p, tca-miR-11617b-3p, tca-miR-11617b-5p, tca-miR-11617c-3p, tca-miR-11617c-5p, tca-miR-11617d-5p, tca-miR-11617e-5p, tca-miR-11617f-5p, tca-miR-11618a-3-3p, tca-miR-11618a-3p, tca-miR-11618a-4-5p, tca-miR-11618a-5-3p, tca-miR-11618a-5p, tca-miR-11618b-3-3p, tca-miR-11618b-3p, tca-miR-11618b-4-3p, tca-miR-11618b-5p, tca-miR-11619a-3-3p, tca-miR-11619a-3p, tca-miR-11619a-5p, tca-miR-11619b-3p, tca-miR-11619c-3p, tca-miR-11619c-5p, tca-miR-11619d-3p, tca-miR-11619d-5p, tca-miR-11619e-3p, tca-miR-11619e-5p, tca-miR-11619f-3p, tca-miR-11619f-5p, tca-miR-11620-3p, tca-miR-11620-5p, tca-miR-11621-3p, tca-miR-11621-5p, tca-miR-11622-3p, tca-miR-11622-5p, tca-miR-11623-3p, tca-miR-11623-5p, tca-miR-11624a-3-3p, tca-miR-11624a-3p, tca-miR-11624a-5p, tca-miR-11624b-3p, tca-miR-11624b-5p, tca-miR-11624c-3p, tca-miR-11624c-5p, tca-miR-11624d-3p, tca-miR-11624d-5p, tca-miR-11625a-3p, tca-miR-11625a-5p, tca-miR-11625b-3p, tca-miR-11625b-5p, tca-miR-11626a-3p, tca-miR-11626a-5p, tca-miR-11626b-3p, tca-miR-11626b-5p, tca-miR-11627-3p, tca-miR-11627-5p, tca-miR-11628-3p, tca-miR-11628-5p, tca-miR-11629-3p, tca-miR-11629-5p, tca-miR-11630-3p, tca-miR-11630-5p, tca-miR-11631-3p, tca-miR-11631-5p, tca-miR-11632-3p, tca-miR-11632-5p, tca-miR-11633-3p, tca-miR-11633-5p, tca-miR-11634-3p, tca-miR-11634-5p, tca-miR-11635-3p, tca-miR-11636-3p, tca-miR-11636-5p, tca-miR-11637-5p, tca-miR-11638-3p, tca-miR-11639-3p, tca-miR-11641-5p, tca-miR-11642-3p, tca-miR-11642-5p, tca-miR-11643-3p, tca-miR-11643-5p, tca-miR-11644-3p, tca-miR-11644-5p, tca-miR-11645-5p, tca-miR-11646-3p, tca-miR-11646-5p, tca-miR-11647-3p, tca-miR-11648-3p, tca-miR-11648-5p, tca-miR-11649-3p, tca-miR-11650-3p, tca-miR-1175-3p, tca-miR-1175-5p, tca-miR-12-3p, tca-miR-12-5p, tca-miR-124-3p, tca-miR-124-5p, tca-miR-125-3p, tca-miR-125-5p, tca-miR-133-3p, tca-miR-133-5p, tca-miR-137-3p, tca-miR-137-5p, tca-miR-13a-3p, tca-miR-13a-5p, tca-miR-13b-3p, tca-miR-13b-5p, tca-miR-14-3p, tca-miR-14-5p, tca-miR-184-3p, tca-miR-184-5p, tca-miR-190-3p, tca-miR-190-5p, tca-miR-193-3p, tca-miR-193-5p, tca-miR-2-1-5p, tca-miR-2-2-5p, tca-miR-2-3-5p, tca-miR-2-3p, tca-miR-210-3p, tca-miR-210-5p, tca-miR-219-3p, tca-miR-219-5p, tca-miR-252a-3p, tca-miR-252a-5p, tca-miR-252b-3p, tca-miR-252b-5p, tca-miR-263a-3p, tca-miR-263a-5p, tca-miR-263b-3p, tca-miR-263b-5p, tca-miR-275-3p, tca-miR-275-5p, tca-miR-276-3p, tca-miR-276-5p, tca-miR-2765-3p, tca-miR-2765-5p, tca-miR-276b-3p, tca-miR-276b-5p, tca-miR-277-3p, tca-miR-277-5p, tca-miR-2788-3p, tca-miR-2788-5p, tca-miR-2796-3p, tca-miR-2796-5p, tca-miR-279a-3p, tca-miR-279a-5p, tca-miR-279b-3p, tca-miR-279b-5p, tca-miR-279c-3p, tca-miR-279c-5p, tca-miR-279d-3p, tca-miR-279d-5p, tca-miR-279e-3p, tca-miR-279e-5p, tca-miR-281-3p, tca-miR-281-5p, tca-miR-282-3p, tca-miR-282-5p, tca-miR-283-3p, tca-miR-283-5p, tca-miR-29-3p, tca-miR-29-5p, tca-miR-2944a-3p, tca-miR-2944a-5p, tca-miR-2944b-3p, tca-miR-2944b-5p, tca-miR-2944c-3p, tca-miR-2944c-5p, tca-miR-304, tca-miR-3049-3p, tca-miR-3049-5p, tca-miR-305-3p, tca-miR-305-5p, tca-miR-307-3p, tca-miR-307-5p, tca-miR-307b-3p, tca-miR-307b-5p, tca-miR-308-3p, tca-miR-308-5p, tca-miR-309a-3p, tca-miR-309a-5p, tca-miR-309b-3p, tca-miR-309b-5p, tca-miR-309c-3p, tca-miR-309c-5p, tca-miR-31-3p, tca-miR-31-5p, tca-miR-315, tca-miR-316-3p, tca-miR-316-5p, tca-miR-317-3p, tca-miR-317-5p, tca-miR-33-3p, tca-miR-33-5p, tca-miR-34-3p, tca-miR-34-5p, tca-miR-3477-3p, tca-miR-3477-5p, tca-miR-375-3p, tca-miR-375-5p, tca-miR-3791-3p, tca-miR-3791-5p, tca-miR-3802-3p, tca-miR-3802-5p, tca-miR-3803-3p, tca-miR-3803-5p, tca-miR-3804a-3p, tca-miR-3804a-5p, tca-miR-3804b-3p, tca-miR-3804b-5p, tca-miR-3805a-3p, tca-miR-3805a-5p, tca-miR-3805b-3p, tca-miR-3805b-5p, tca-miR-3806-3p, tca-miR-3806-5p, tca-miR-3806b-3p, tca-miR-3806b-5p, tca-miR-3806c-3p, tca-miR-3807-3p, tca-miR-3807-5p, tca-miR-3808-3p, tca-miR-3808-5p, tca-miR-3809-3p, tca-miR-3809-5p, tca-miR-3810-3p, tca-miR-3810-5p, tca-miR-3811a-1-5p, tca-miR-3811a-3-5p, tca-miR-3811a-3p, tca-miR-3811a-5-5p, tca-miR-3811a-5p, tca-miR-3811b-3p, tca-miR-3811b-5p, tca-miR-3812-3p, tca-miR-3812-5p, tca-miR-3813-3p, tca-miR-3813-5p, tca-miR-3814-3p, tca-miR-3814-5p, tca-miR-3815-3p, tca-miR-3815-5p, tca-miR-3816-3p, tca-miR-3816-5p, tca-miR-3817-3p, tca-miR-3817-5p, tca-miR-3818-3p, tca-miR-3818-5p, tca-miR-3819-5p, tca-miR-3820-3p, tca-miR-3820-5p, tca-miR-3821-3p, tca-miR-3821-5p, tca-miR-3822-3p, tca-miR-3822-5p, tca-miR-3823-3p, tca-miR-3823-5p, tca-miR-3824-3p, tca-miR-3824-5p, tca-miR-3825-3p, tca-miR-3825-5p, tca-miR-3826-3p, tca-miR-3826-5p, tca-miR-3827-3p, tca-miR-3827-5p, tca-miR-3828-3p, tca-miR-3828-5p, tca-miR-3829-3p, tca-miR-3829-5p, tca-miR-3830-3p, tca-miR-3830-5p, tca-miR-3831-3p, tca-miR-3831-5p, tca-miR-3832-3p, tca-miR-3832-5p, tca-miR-3833-3p, tca-miR-3833-5p, tca-miR-3834-3p, tca-miR-3834-5p, tca-miR-3835-3p, tca-miR-3835-5p, tca-miR-3836a-3p, tca-miR-3836a-5p, tca-miR-3836b-3p, tca-miR-3836b-5p, tca-miR-3836c-1-3p, tca-miR-3836c-2-3p, tca-miR-3836c-3-5p, tca-miR-3836c-3p, tca-miR-3836c-5p, tca-miR-3836d-3p, tca-miR-3836d-5p, tca-miR-3836e-3p, tca-miR-3836e-5p, tca-miR-3836f-3p, tca-miR-3836f-5p, tca-miR-3836g-3p, tca-miR-3836g-5p, tca-miR-3836 h-3p, tca-miR-3836i-3p, tca-miR-3836i-5p, tca-miR-3836j-3p, tca-miR-3837-3p, tca-miR-3837-5p, tca-miR-3838-3p, tca-miR-3838-5p, tca-miR-3839-3p, tca-miR-3839-5p, tca-miR-3840-3p, tca-miR-3840-5p, tca-miR-3841-3p, tca-miR-3841-5p, tca-miR-3842-3p, tca-miR-3842-5p, tca-miR-3843-3p, tca-miR-3843-5p, tca-miR-3844-3p, tca-miR-3844-5p, tca-miR-3845-3p, tca-miR-3845-5p, tca-miR-3846-3p, tca-miR-3846-5p, tca-miR-3847-3p, tca-miR-3847-5p, tca-miR-3848-3p, tca-miR-3848-5p, tca-miR-3849-3p, tca-miR-3849-5p, tca-miR-3850-3p, tca-miR-3850-5p, tca-miR-3851a-3p, tca-miR-3851a-5p, tca-miR-3851b-3p, tca-miR-3851b-5p, tca-miR-3851c-3p, tca-miR-3851c-5p, tca-miR-3851d-2-5p, tca-miR-3851d-3p, tca-miR-3851d-5p, tca-miR-3851e-3p, tca-miR-3851e-5p, tca-miR-3851f-3p, tca-miR-3851f-5p, tca-miR-3851g-2-3p, tca-miR-3851g-3p, tca-miR-3851g-5p, tca-miR-3851 h-3p, tca-miR-3851 h-5p, tca-miR-3851i-3p, tca-miR-3851i-5p, tca-miR-3851j-3p, tca-miR-3851j-5p, tca-miR-3851k-3p, tca-miR-3851k-5p, tca-miR-38511-2-3p, tca-miR-38511-3-5p, tca-miR-38511-3p, tca-miR-38511-4-3p, tca-miR-38511-5p, tca-miR-3851m-3p, tca-miR-3851m-4-3p, tca-miR-3851m-5p, tca-miR-3851n-3p, tca-miR-3851n-5p, tca-miR-38510-2-5p, tca-miR-38510-3p, tca-miR-38510-5p, tca-miR-3851p-3p, tca-miR-3851p-5p, tca-miR-3851q-3p, tca-miR-3851q-5p, tca-miR-3851r-3p, tca-miR-3851r-5p, tca-miR-3852-3p, tca-miR-3852-5p, tca-miR-3853-3p, tca-miR-3853-5p, tca-miR-3854-3p, tca-miR-3854-5p, tca-miR-3855-3p, tca-miR-3855-5p, tca-miR-3856-3p, tca-miR-3856-5p, tca-miR-3857-3p, tca-miR-3857-5p, tca-miR-3858-3p, tca-miR-3858-5p, tca-miR-3858a-3p, tca-miR-3858a-5p, tca-miR-3858b-3p, tca-miR-3858b-5p, tca-miR-3859-3p, tca-miR-3859-5p, tca-miR-3860-3p, tca-miR-3860-5p, tca-miR-3861-3p, tca-miR-3861-5p, tca-miR-3862-3p, tca-miR-3862-5p, tca-miR-3863-3p, tca-miR-3863-5p, tca-miR-3864-3p, tca-miR-3864-5p, tca-miR-3865-3p, tca-miR-3865-5p, tca-miR-3866-3p, tca-miR-3866-5p, tca-miR-3867-3p, tca-miR-3867-5p, tca-miR-3868-3p, tca-miR-3868-5p, tca-miR-3869-3p, tca-miR-3869-5p, tca-miR-3870-3p, tca-miR-3870-5p, tca-miR-3871-3p, tca-miR-3871-5p, tca-miR-3872-3p, tca-miR-3872-5p, tca-miR-3873-3p, tca-miR-3873-5p, tca-miR-3874-3p, tca-miR-3874-5p, tca-miR-3875-3p, tca-miR-3875-5p, tca-miR-3876-3p, tca-miR-3876-5p, tca-miR-3877-3p, tca-miR-3877-5p, tca-miR-3878-3p, tca-miR-3878-5p, tca-miR-3879-3p, tca-miR-3879-5p, tca-miR-3880-3p, tca-miR-3880-5p, tca-miR-3881-3p, tca-miR-3881-5p, tca-miR-3882-3p, tca-miR-3882-5p, tca-miR-3883-3p, tca-miR-3883-5p, tca-miR-3884-3p, tca-miR-3884-5p, tca-miR-3885-3p, tca-miR-3885-5p, tca-miR-3886-3p, tca-miR-3886-5p, tca-miR-3887-3p, tca-miR-3887-5p, tca-miR-3888-3p, tca-miR-3888-5p, tca-miR-3889-3p, tca-miR-3889-5p, tca-miR-3890-3p, tca-miR-3890-5p, tca-miR-3891-3p, tca-miR-3891-5p, tca-miR-3892-3p, tca-miR-3892-5p, tca-miR-3893-3p, tca-miR-3893-5p, tca-miR-3894-3p, tca-miR-3894-5p, tca-miR-3895-3p, tca-miR-3895-5p, tca-miR-3896-3p, tca-miR-3896-5p, tca-miR-3897-3p, tca-miR-3897-5p, tca-miR-3898-3p, tca-miR-3898-5p, tca-miR-3899-3p, tca-miR-3899-5p, tca-miR-3900-3p, tca-miR-3900-5p, tca-miR-3901-3p, tca-miR-3901-5p, tca-miR-3902-3p, tca-miR-3902-5p, tca-miR-3903-3p, tca-miR-3904-3p, tca-miR-3904-5p, tca-miR-3905-5p, tca-miR-6007-3p, tca-miR-6007-5p, tca-miR-6008-3p, tca-miR-6008-5p, tca-miR-6009-3p, tca-miR-6009-5p, tca-miR-6010-3p, tca-miR-6010-5p, tca-miR-6011-3p, tca-miR-6011-5p, tca-miR-6012-3p, tca-miR-6012-5p, tca-miR-6013-3p, tca-miR-6013-5p, tca-miR-6014-3p, tca-miR-6014-5p, tca-miR-6015-3p, tca-miR-6015-5p, tca-miR-6016-3p, tca-miR-6016-5p, tca-miR-6016b-3p, tca-miR-6016b-5p, tca-miR-6017-3p, tca-miR-6017-5p, tca-miR-6018-3p, tca-miR-6018-5p, tca-miR-7-3p, tca-miR-7-5p, tca-miR-71-3p, tca-miR-71-5p, tca-miR-750-3p, tca-miR-750-5p, tca-miR-8-3p, tca-miR-8-5p, tca-miR-87a-3p, tca-miR-87a-5p, tca-miR-87b-3p, tca-miR-87b-5p, tca-miR-927a-3p, tca-miR-927a-5p, tca-miR-927b-3p, tca-miR-927b-5p, tca-miR-927c-3p, tca-miR-927c-5p, tca-miR-929-3p, tca-miR-929-5p, tca-miR-92a-3p, tca-miR-92a-5p, tca-miR-92b-3p, tca-miR-92b-5p, tca-miR-92c-3p, tca-miR-92c-5p, tca-miR-932-3p, tca-miR-932-5p, tca-miR-965-3p, tca-miR-965-5p, tca-miR-970-3p, tca-miR-970-5p, tca-miR-971b-3p, tca-miR-971b-5p, tca-miR-980-3p, tca-miR-980-5p, tca-miR-981, tca-miR-989-3p, tca-miR-989-5p, tca-miR-993-3p, tca-miR-993-5p, tca-miR-995-3p, tca-miR-995-5p, tca-miR-998-3p, tca-miR-998-5p, tca-miR-9a-3p, tca-miR-9a-5p, tca-miR-9b-3p, tca-miR-9b-5p, tca-miR-9c-3p, tca-miR-9c-5p, tca-miR-9d-3p, tca-miR-9d-5p, tca-miR-9e-3p, tca-miR-9e-5p, tca-miR-iab-4-3p, tca-miR-iab-4-5p, tca-miR-iab-8-3p, tca-miR-iab-8-5p, tcc-miR156a, tcc-miR156b, tcc-miR156c, tcc-miR156d, tcc-miR156e, tcc-miR156f, tcc-miR156g, tcc-miR160a, tcc-miR160b, tcc-miR160c, tcc-miR162, tcc-miR164a, tcc-miR164b, tcc-miR164c, tcc-miR166a, tcc-miR166b, tcc-miR166c, tcc-miR166d, tcc-miR167a, tcc-miR167b, tcc-miR167c, tcc-miR168, tcc-miR169a, tcc-miR169b, tcc-miR169c, tcc-miR169d, tcc-miR169e, tcc-miR169f, tcc-miR169g, tcc-miR169h, tcc-miR169i, tcc-miR169j, tcc-miR169k, tcc-miR1691, tcc-miR169m, tcc-miR169n, tcc-miR171a, tcc-miR171b, tcc-miR171c, tcc-miR171d, tcc-miR171e, tcc-miR171f, tcc-miR171g, tcc-miR171h, tcc-miR172a, tcc-miR172b, tcc-miR172c, tcc-miR172d, tcc-miR172e, tcc-miR2111, tcc-miR319, tcc-miR390a, tcc-miR390b, tcc-miR393a, tcc-miR393b, tcc-miR394a, tcc-miR394b, tcc-miR395a, tcc-miR395b, tcc-miR396a, tcc-miR396b, tcc-miR396c, tcc-miR396d, tcc-miR396e, tcc-miR397, tcc-miR398a, tcc-miR398b, tcc-miR399a, tcc-miR399b, tcc-miR399c, tcc-miR399d, tcc-miR399e, tcc-miR399f, tcc-miR399g, tcc-miR399h, tcc-miR399i, tcc-miR403a, tcc-miR403b, tcc-miR530a, tcc-miR530b, tcc-miR535, tcc-miR827, tcf-bantam, tcf-let-7-3p, tcf-let-7-5p, tcf-miR-1, tcf-miR-10-3p, tcf-miR-10-5p, tcf-miR-100, tcf-miR-10294, tcf-miR-10295, tcf-miR-10296, tcf-miR-10297, tcf-miR-10298a, tcf-miR-10298b, tcf-miR-10299, tcf-miR-10300, tcf-miR-10301, tcf-miR-10302, tcf-miR-10303a, tcf-miR-10303b, tcf-miR-10304, tcf-miR-10305, tcf-miR-10306, tcf-miR-10307, tcf-miR-10308, tcf-miR-10309, tcf-miR-10310, tcf-miR-10311, tcf-miR-10312, tcf-miR-10313, tcf-miR-10314, tcf-miR-10315a, tcf-miR-10315b, tcf-miR-10316a, tcf-miR-10316b, tcf-miR-10316c, tcf-miR-10317, tcf-miR-10318, tcf-miR-10319, tcf-miR-10320, tcf-miR-10321, tcf-miR-10322, tcf-miR-10323, tcf-miR-10324, tcf-miR-10325, tcf-miR-10326, tcf-miR-10327, tcf-miR-10328, tcf-miR-10329, tcf-miR-10330, tcf-miR-10331, tcf-miR-10332, tcf-miR-10333, tcf-miR-10334, tcf-miR-10335, tcf-miR-10336, tcf-miR-10337, tcf-miR-10338, tcf-miR-10339, tcf-miR-10340, tcf-miR-10341, tcf-miR-10342, tcf-miR-10343, tcf-miR-10344, tcf-miR-10345, tcf-miR-10346, tcf-miR-10347, tcf-miR-10348, tcf-miR-10349, tcf-miR-10350, tcf-miR-10351, tcf-miR-10352, tcf-miR-10353, tcf-miR-10354, tcf-miR-10402, tcf-miR-10403, tcf-miR-1175-3p, tcf-miR-1175-5p, tcf-miR-12, tcf-miR-124-3p, tcf-miR-124-5p, tcf-miR-125, tcf-miR-13, tcf-miR-133, tcf-miR-137, tcf-miR-153-3p, tcf-miR-153-5p, tcf-miR-184-3p, tcf-miR-184-5p, tcf-miR-190, tcf-miR-193, tcf-miR-210-3p, tcf-miR-210-5p, tcf-miR-219, tcf-miR-252a-3p, tcf-miR-252a-5p, tcf-miR-252b, tcf-miR-263a, tcf-miR-263b, tcf-miR-275, tcf-miR-276-3p, tcf-miR-276-5p, tcf-miR-277, tcf-miR-278, tcf-miR-2788, tcf-miR-279a, tcf-miR-279b, tcf-miR-279c, tcf-miR-279d, tcf-miR-279e, tcf-miR-279f, tcf-miR-281-3p, tcf-miR-281-5p, tcf-miR-282, tcf-miR-283, tcf-miR-285, tcf-miR-2944a, tcf-miR-2944b, tcf-miR-2a-3p, tcf-miR-2a-5p, tcf-miR-2b, tcf-miR-305, tcf-miR-307, tcf-miR-309, tcf-miR-31, tcf-miR-315-3p, tcf-miR-315-5p, tcf-miR-316, tcf-miR-317, tcf-miR-318, tcf-miR-33, tcf-miR-34, tcf-miR-3477, tcf-miR-375, tcf-miR-3791, tcf-miR-7, tcf-miR-71-3p, tcf-miR-71-5p, tcf-miR-745, tcf-miR-750, tcf-miR-79, tcf-miR-8-3p, tcf-miR-8-5p, tcf-miR-87, tcf-miR-87b, tcf-miR-92a, tcf-miR-92b, tcf-miR-96, tcf-miR-965, tcf-miR-981, tcf-miR-993-3p, tcf-miR-993-5p, tcf-miR-995, tcf-miR-996, tcf-miR-998, tcf-miR-9a-3p, tcf-miR-9a-5p, tcf-miR-9b-3p, tcf-miR-9b-5p, tcf-miR-iab-4, tcf-miR-iab-8, tch-let-7a-3p, tch-let-7a-5p, tch-let-7e-5p, tch-let-7f-5p, tch-let-7g-5p, tch-let-7i-5p, tch-miR-100-5p, tch-miR-101, tch-miR-103a-3p, tch-miR-107, tch-miR-10b-5p, tch-miR-122-5p, tch-miR-1247-5p, tch-miR-125a-5p, tch-miR-125b-5p, tch-miR-127-3p, tch-miR-1277-5p, tch-miR-128, tch-miR-129-5p, tch-miR-1301, tch-miR-1306-3p, tch-miR-1306-5p, tch-miR-130a-3p, tch-miR-135a-5p, tch-miR-135b-5p, tch-miR-136-5p, tch-miR-139-5p, tch-miR-140-5p, tch-miR-141-3p, tch-miR-142, tch-miR-143-3p, tch-miR-146a-5p, tch-miR-147b, tch-miR-148a-3p, tch-miR-148b-3p, tch-miR-152, tch-miR-155-5p, tch-miR-15a-5p, tch-miR-15b-5p, tch-miR-17-5p, tch-miR-181a-5p, tch-miR-181b-5p, tch-miR-181c-5p, tch-miR-181d, tch-miR-183-5p, tch-miR-184, tch-miR-185-5p, tch-miR-186-5p, tch-miR-188-3p, tch-miR-188-5p, tch-miR-190a, tch-miR-190b, tch-miR-191-5p, tch-miR-192-5p, tch-miR-193a-3p, tch-miR-193b-3p, tch-miR-194-5p, tch-miR-195-5p, tch-miR-196a-5p, tch-miR-199a-3p, tch-miR-199b-3p, tch-miR-19a-3p, tch-miR-19b-3p, tch-miR-200a-3p, tch-miR-200b-3p, tch-miR-200c-3p, tch-miR-20a-5p, tch-miR-21-5p, tch-miR-210, tch-miR-218-5p, tch-miR-219-5p, tch-miR-22-3p, tch-miR-221-3p, tch-miR-222-3p, tch-miR-23b-3p, tch-miR-25-3p, tch-miR-26a-2-5p, tch-miR-26a-5p, tch-miR-26b-5p, tch-miR-27a-3p, tch-miR-27b-3p, tch-miR-28-5p, tch-miR-296-3p, tch-miR-29a-3p, tch-miR-29b-3p, tch-miR-29c-3p, tch-miR-30a-5p, tch-miR-30b-5p, tch-miR-30c-5p, tch-miR-32-5p, tch-miR-324-5p, tch-miR-326, tch-miR-328, tch-miR-330-3p, tch-miR-335-5p, tch-miR-33a-5p, tch-miR-342-3p, tch-miR-34a-5p, tch-miR-34c-5p, tch-miR-361-5p, tch-miR-363-3p, tch-miR-365a-3p, tch-miR-365b-3p, tch-miR-369-3p, tch-miR-374a-5p, tch-miR-374b-5p, tch-miR-376a-3p, tch-miR-376b-3p, tch-miR-376c-3p, tch-miR-378a-3p, tch-miR-381-3p, tch-miR-409-3p, tch-miR-411-5p, tch-miR-423-3p, tch-miR-424-5p, tch-miR-425-5p, tch-miR-432-5p, tch-miR-450a-5p, tch-miR-455-3p, tch-miR-486-3p, tch-miR-491-5p, tch-miR-497-5p, tch-miR-499a-5p, tch-miR-503, tch-miR-504, tch-miR-505-3p, tch-miR-532-5p, tch-miR-539-3p, tch-miR-542-3p, tch-miR-592, tch-miR-628-5p, tch-miR-652-3p, tch-miR-6529, tch-miR-656, tch-miR-671-5p, tch-miR-7-5p, tch-miR-708-5p, tch-miR-885-3p, tch-miR-9-5p, tch-miR-92a-3p, tch-miR-93-5p, tch-miR-95, tch-miR-9770, tch-miR-9771a, tch-miR-9771b, tch-miR-9771c, tch-miR-9771d, tch-miR-9771e, tch-miR-9771f, tch-miR-9771g, tch-miR-9771h, tch-miR-9771i, tch-miR-9771j, tch-miR-9771k, tch-miR-97711, tch-miR-9771m, tch-miR-9771n, tch-miR-97710, tch-miR-9771p, tch-miR-98-5p, tch-miR-99a-5p, tch-miR-99b-5p, tgu-let-7a-1-3p, tgu-let-7a-2-3p, tgu-let-7a-4-3p, tgu-let-7a-5p, tgu-let-7b-3p, tgu-let-7b-5p, tgu-let-7c-3p, tgu-let-7c-5p, tgu-let-7d-3p, tgu-let-7d-5p, tgu-let-7e-3p, tgu-let-7e-5p, tgu-let-7f-3p, tgu-let-7f-5p, tgu-let-7g-3p, tgu-let-7g-5p, tgu-let-7i-3p, tgu-let-7i-5p, tgu-miR-1-1-5p, tgu-miR-1-2-5p, tgu-miR-1-3p, tgu-miR-100-3p, tgu-miR-100-5p, tgu-miR-101-1-5p, tgu-miR-101-2-5p, tgu-miR-101-3p, tgu-miR-103-3p, tgu-miR-103-5p, tgu-miR-106-3p, tgu-miR-106-5p, tgu-miR-107, tgu-miR-10a-3p, tgu-miR-10a-5p, tgu-miR-122-3p, tgu-miR-122-5p, tgu-miR-124-3p, tgu-miR-124-5p, tgu-miR-125-1-3p, tgu-miR-125-2-3p, tgu-miR-125-5p, tgu-miR-126-3p, tgu-miR-126-5p, tgu-miR-128-1-5p, tgu-miR-128-2-5p, tgu-miR-128-3p, tgu-miR-129-3p, tgu-miR-129-5p, tgu-miR-130a-3p, tgu-miR-130a-5p, tgu-miR-130b-3p, tgu-miR-130b-5p, tgu-miR-130c-3p, tgu-miR-130c-5p, tgu-miR-132, tgu-miR-1329-3p, tgu-miR-1329-5p, tgu-miR-133-3p, tgu-miR-133-5p, tgu-miR-135a-1-3p, tgu-miR-135a-2-3p, tgu-miR-135a-5p, tgu-miR-135b, tgu-miR-137-3p, tgu-miR-137-5p, tgu-miR-138-1-3p, tgu-miR-138-2-3p, tgu-miR-138-5p, tgu-miR-1388-3p, tgu-miR-1388-5p, tgu-miR-139-3p, tgu-miR-139-5p, tgu-miR-1397-3p, tgu-miR-1397-5p, tgu-miR-140-3p, tgu-miR-140-5p, tgu-miR-142-3p, tgu-miR-142-5p, tgu-miR-144-3p, tgu-miR-144-5p, tgu-miR-1451-3p, tgu-miR-1451-5p, tgu-miR-1460, tgu-miR-1467-3p, tgu-miR-1467-5p, tgu-miR-146a-3p, tgu-miR-146a-5p, tgu-miR-146b-3p, tgu-miR-146b-5p, tgu-miR-146c, tgu-miR-147, tgu-miR-148a-3p, tgu-miR-148a-5p, tgu-miR-148b, tgu-miR-153-1-5p, tgu-miR-153-2-5p, tgu-miR-153-3p, tgu-miR-155-3p, tgu-miR-155-5p, tgu-miR-1559, tgu-miR-15a-3p, tgu-miR-15a-5p, tgu-miR-15b-3p, tgu-miR-15b-5p, tgu-miR-15c-3p, tgu-miR-15c-5p, tgu-miR-1641, tgu-miR-1662, tgu-miR-1669, tgu-miR-1677-3p, tgu-miR-1677-5p, tgu-miR-16a-3p, tgu-miR-16a-5p, tgu-miR-16b-3p, tgu-miR-16b-5p, tgu-miR-16c, tgu-miR-1729-3p, tgu-miR-1729-5p, tgu-miR-1743, tgu-miR-1744, tgu-miR-1756, tgu-miR-1759, tgu-miR-1781, tgu-miR-1784-3p, tgu-miR-1784-5p, tgu-miR-1788, tgu-miR-1789, tgu-miR-1791, tgu-miR-17a-3p, tgu-miR-17a-5p, tgu-miR-1803-3p, tgu-miR-1803-5p, tgu-miR-1805-3p, tgu-miR-1805-5p, tgu-miR-181a-1-3p, tgu-miR-181a-2-3p, tgu-miR-181a-5p, tgu-miR-181b-1-3p, tgu-miR-181b-2-3p, tgu-miR-181b-5p, tgu-miR-183, tgu-miR-184, tgu-miR-187-3p, tgu-miR-187-5p, tgu-miR-18a, tgu-miR-18b, tgu-miR-190-3p, tgu-miR-190-5p, tgu-miR-192, tgu-miR-193a-3p, tgu-miR-193a-5p, tgu-miR-193b-3p, tgu-miR-193b-5p, tgu-miR-194-3p, tgu-miR-194-5p, tgu-miR-196, tgu-miR-199-3p, tgu-miR-199-5p, tgu-miR-19a-3p, tgu-miR-19a-5p, tgu-miR-19b-1-5p, tgu-miR-19b-2-5p, tgu-miR-19b-3p, tgu-miR-200a-3p, tgu-miR-200a-5p, tgu-miR-200b-3p, tgu-miR-200b-5p, tgu-miR-202, tgu-miR-203-3p, tgu-miR-203-5p, tgu-miR-204, tgu-miR-205, tgu-miR-20a-3p, tgu-miR-20a-5p, tgu-miR-20b-3p, tgu-miR-20b-5p, tgu-miR-21-3p, tgu-miR-21-5p, tgu-miR-2131, tgu-miR-214-3p, tgu-miR-214-5p, tgu-miR-215-3p, tgu-miR-215-5p, tgu-miR-216a-3p, tgu-miR-216a-5p, tgu-miR-216b-3p, tgu-miR-216b-5p, tgu-miR-217-3p, tgu-miR-217-5p, tgu-miR-218-3p, tgu-miR-218-5p, tgu-miR-2188-3p, tgu-miR-2188-5p, tgu-miR-219a, tgu-miR-219b, tgu-miR-221-3p, tgu-miR-221-5p, tgu-miR-222-3p, tgu-miR-222-5p, tgu-miR-223, tgu-miR-23-3p, tgu-miR-23-5p, tgu-miR-24-3p, tgu-miR-24-5p, tgu-miR-25, tgu-miR-26-3p, tgu-miR-26-5p, tgu-miR-27-3p, tgu-miR-27-5p, tgu-miR-2954-3p, tgu-miR-2954-5p, tgu-miR-2955-3p, tgu-miR-2955-5p, tgu-miR-2956, tgu-miR-2958, tgu-miR-2959, tgu-miR-2960, tgu-miR-2961, tgu-miR-2962-3p, tgu-miR-2962-5p, tgu-miR-2963-3p, tgu-miR-2963-5p, tgu-miR-2965, tgu-miR-2966, tgu-miR-2967, tgu-miR-2968, tgu-miR-2969, tgu-miR-2970-3p, tgu-miR-2970-5p, tgu-miR-2971, tgu-miR-2972, tgu-miR-2973-3p, tgu-miR-2973-5p, tgu-miR-2974, tgu-miR-2975, tgu-miR-2976, tgu-miR-2977, tgu-miR-2978, tgu-miR-2979-3p, tgu-miR-2979-5p, tgu-miR-2980, tgu-miR-2981, tgu-miR-2982, tgu-miR-2983, tgu-miR-2984-3p, tgu-miR-2984-5p, tgu-miR-2985, tgu-miR-2986, tgu-miR-2987-3p, tgu-miR-2987-5p, tgu-miR-2988, tgu-miR-2989, tgu-miR-2991, tgu-miR-2992, tgu-miR-2993, tgu-miR-2994, tgu-miR-2995, tgu-miR-2996, tgu-miR-2997-3p, tgu-miR-2997-5p, tgu-miR-29a-1-5p, tgu-miR-29a-2-5p, tgu-miR-29a-3p, tgu-miR-29b-1-5p, tgu-miR-29b-2-5p, tgu-miR-29b-3p, tgu-miR-301-3p, tgu-miR-301-5p, tgu-miR-30a-3p, tgu-miR-30a-5p, tgu-miR-30b-3p, tgu-miR-30b-5p, tgu-miR-30c-3p, tgu-miR-30c-5p, tgu-miR-30d-3p, tgu-miR-30d-5p, tgu-miR-30e, tgu-miR-31, tgu-miR-32, tgu-miR-33-3p, tgu-miR-33-5p, tgu-miR-338-3p, tgu-miR-338-5p, tgu-miR-34a, tgu-miR-34b, tgu-miR-34c-3p, tgu-miR-34c-5p, tgu-miR-363-3p, tgu-miR-363-5p, tgu-miR-365-1-5p, tgu-miR-365-2-5p, tgu-miR-365-3p, tgu-miR-367, tgu-miR-375, tgu-miR-383-3p, tgu-miR-383-5p, tgu-miR-425-3p, tgu-miR-425-5p, tgu-miR-451, tgu-miR-454-3p, tgu-miR-454-5p, tgu-miR-455-3p, tgu-miR-455-5p, tgu-miR-456-3p, tgu-miR-456-5p, tgu-miR-458-3p, tgu-miR-458-5p, tgu-miR-460a-3p, tgu-miR-460a-5p, tgu-miR-460b-3p, tgu-miR-460b-5p, tgu-miR-489-3p, tgu-miR-489-5p, tgu-miR-490-3p, tgu-miR-490-5p, tgu-miR-499-3p, tgu-miR-499-5p, tgu-miR-551-3p, tgu-miR-551-5p, tgu-miR-7-1-3p, tgu-miR-7-4-3p, tgu-miR-7-5p, tgu-miR-7643-3p, tgu-miR-7643-5p, tgu-miR-7644-3p, tgu-miR-7644-5p, tgu-miR-7645-3p, tgu-miR-7645-5p, tgu-miR-9-3p, tgu-miR-9-5p, tgu-miR-92-1-5p, tgu-miR-92-2-5p, tgu-miR-92-3p, tgu-miR-99-3p, tgu-miR-99-5p, tni-let-7a, tni-let-7b, tni-let-7d, tni-let-7e, tni-let-7g, tni-let-7h, tni-let-7i, tni-let-7j, tni-miR-1, tni-miR-100, tni-miR-101a, tni-miR-101b, tni-miR-103, tni-miR-107, tni-miR-10b, tni-miR-10c, tni-miR-10d, tni-miR-122, tni-miR-124, tni-miR-125a, tni-miR-125b, tni-miR-126, tni-miR-128, tni-miR-129, tni-miR-130, tni-miR-132, tni-miR-133, tni-miR-135a, tni-miR-135b, tni-miR-137, tni-miR-138, tni-miR-140, tni-miR-142a, tni-miR-142b, tni-miR-144, tni-miR-148, tni-miR-152, tni-miR-153a, tni-miR-153b, tni-miR-15a, tni-miR-15b, tni-miR-16, tni-miR-17, tni-miR-18, tni-miR-181a-3p, tni-miR-181a-5p, tni-miR-181b, tni-miR-182, tni-miR-183, tni-miR-184, tni-miR-187, tni-miR-190, tni-miR-192, tni-miR-193, tni-miR-194, tni-miR-196a, tni-miR-196b, tni-miR-199, tni-miR-19a, tni-miR-19b, tni-miR-20, tni-miR-200a, tni-miR-200b, tni-miR-202, tni-miR-203, tni-miR-204a, tni-miR-204b, tni-miR-205, tni-miR-21, tni-miR-210, tni-miR-212, tni-miR-214, tni-miR-216a, tni-miR-216b, tni-miR-217, tni-miR-218a, tni-miR-218b, tni-miR-219, tni-miR-221, tni-miR-222, tni-miR-223, tni-miR-22a, tni-miR-22b, tni-miR-23a, tni-miR-23b, tni-miR-24, tni-miR-25, tni-miR-26, tni-miR-27b, tni-miR-27c, tni-miR-27e, tni-miR-29a, tni-miR-29b, tni-miR-301, tni-miR-30b, tni-miR-30c, tni-miR-30d, tni-miR-338, tni-miR-365, tni-miR-375, tni-miR-429, tni-miR-455, tni-miR-458, tni-miR-460, tni-miR-489, tni-miR-7, tni-miR-9, tni-miR-92, tni-miR-96, tre-miR-5045, ttu-miR160, ttv-miR-T1-3p, ttv-miR-T1-5p, tur-miR-1-3p, tur-miR-1-5p, tur-miR-10-3p, tur-miR-10-5p, tur-miR-124-1-5p, tur-miR-124-2-5p, tur-miR-124-3p, tur-miR-12a-3p, tur-miR-12a-5p, tur-miR-12b-3p, tur-miR-12b-5p, tur-miR-133-3p, tur-miR-133-5p, tur-miR-137-3p, tur-miR-137-5p, tur-miR-184-3p, tur-miR-184-5p, tur-miR-190-3p, tur-miR-190-5p, tur-miR-2-3p, tur-miR-2-5p, tur-miR-210-3p, tur-miR-210-5p, tur-miR-252-3p, tur-miR-252-5p, tur-miR-263a-3p, tur-miR-263a-5p, tur-miR-263b-3p, tur-miR-263b-5p, tur-miR-276-3p, tur-miR-276-5p, tur-miR-278-3p, tur-miR-278-5p, tur-miR-279-3p, tur-miR-279-5p, tur-miR-281-3p, tur-miR-281-5p, tur-miR-305-3p, tur-miR-305-5p, tur-miR-307-3p, tur-miR-307-5p, tur-miR-317-3p, tur-miR-317-5p, tur-miR-34-3p, tur-miR-34-5p, tur-miR-3931-3p, tur-miR-3931-5p, tur-miR-5727-3p, tur-miR-5727-5p, tur-miR-5728-1-5p, tur-miR-5728-2-5p, tur-miR-5728-3p, tur-miR-5729a-3p, tur-miR-5729a-5p, tur-miR-5729b-3p, tur-miR-5729b-5p, tur-miR-5730-3p, tur-miR-5730-5p, tur-miR-5731-3p, tur-miR-5731-5p, tur-miR-5732-3p, tur-miR-5732-5p, tur-miR-5733-3p, tur-miR-5733-5p, tur-miR-5734-3p, tur-miR-5734-5p, tur-miR-5735-3p, tur-miR-5735-5p, tur-miR-5736a-3p, tur-miR-5736a-5p, tur-miR-5736b-3p, tur-miR-5736b-5p, tur-miR-5737-3p, tur-miR-5737-5p, tur-miR-5738-3p, tur-miR-5738-5p, tur-miR-7-3p, tur-miR-7-5p, tur-miR-71-3p, tur-miR-71-5p, tur-miR-745-3p, tur-miR-745-5p, tur-miR-87-3p, tur-miR-87-5p, tur-miR-9-3p, tur-miR-9-5p, tur-miR-92-3p, tur-miR-92-5p, tur-miR-993a-3p, tur-miR-993a-5p, tur-miR-993b-3p, tur-miR-993b-5p, vca-miR10202a-3p, vca-miR10202a-5p, vca-miR10202b-3p, vca-miR10202b-5p, vca-miR10203-3p, vca-miR10203-5p, vca-miR10204-3p, vca-miR10204-5p, vca-miR10205-3p, vca-miR10205-5p, vca-miR10206a-3p, vca-miR10206a-5p, vca-miR10206b-3p, vca-miR10206b-5p, vca-miR10207-3p, vca-miR10207-5p, vca-miR10208-3p, vca-miR10208-5p, vca-miR10209-3p, vca-miR10209-5p, vca-miR10210-3p, vca-miR10210-5p, vca-miR1432-3p, vca-miR1432-5p, vca-miR156a-3p, vca-miR156a-5p, vca-miR156b-3p, vca-miR156b-5p, vca-miR159-3p, vca-miR159-5p, vca-miR160-5p, vca-miR166a-3p, vca-miR166a-5p, vca-miR166b-3p, vca-miR166b-5p, vca-miR166c-3p, vca-miR166c-5p, vca-miR167a-3p, vca-miR167a-5p, vca-miR167b-3p, vca-miR167b-5p, vca-miR168a-3p, vca-miR168a-5p, vca-miR168b-3p, vca-miR168b-5p, vca-miR172a-3p, vca-miR172a-5p, vca-miR172b-3p, vca-miR172b-5p, vca-miR391-3p, vca-miR391-5p, vca-miR393-3p, vca-miR393-5p, vca-miR396-3p, vca-miR396-5p, vca-miR396b-3p, vca-miR396b-5p, vca-miR397-3p, vca-miR397-5p, vca-miR408-3p, vca-miR408-5p, vca-miR528-3p, vca-miR528-5p, vca-miR535-3p, vca-miR535-5p, vun-miR1507a, vun-miR1507b, vun-miR156a, vun-miR156b, vun-miR160, vun-miR162, vun-miR164, vun-miR168, vun-miR169, vun-miR172, vun-miR2118, vun-miR319a, vun-miR319b, vun-miR395, vun-miR399a, vun-miR399b, vun-miR408, vun-miR482, vvi-miR156a, vvi-miR156b, vvi-miR156c, vvi-miR156d, vvi-miR156e, vvi-miR156f, vvi-miR156g, vvi-miR156h, vvi-miR156i, vvi-miR159a, vvi-miR159b, vvi-miR159c, vvi-miR160a, vvi-miR160b, vvi-miR160c, vvi-miR160d, vvi-miR160e, vvi-miR162, vvi-miR164a, vvi-miR164b, vvi-miR164c, vvi-miR164d, vvi-miR166a, vvi-miR166b, vvi-miR166c, vvi-miR166d, vvi-miR166e, vvi-miR166f, vvi-miR166g, vvi-miR166h, vvi-miR167a, vvi-miR167b, vvi-miR167c, vvi-miR167d, vvi-miR167e, vvi-miR168, vvi-miR169a, vvi-miR169b, vvi-miR169c, vvi-miR169d, vvi-miR169e, vvi-miR169f, vvi-miR169g, vvi-miR169h, vvi-miR169i, vvi-miR169j, vvi-miR169k, vvi-miR1691, vvi-miR169m, vvi-miR169n, vvi-miR1690, vvi-miR169p, vvi-miR169q, vvi-miR169r, vvi-miR169s, vvi-miR169t, vvi-miR169u, vvi-miR169v, vvi-miR169w, vvi-miR169x, vvi-miR169y, vvi-miR171a, vvi-miR171b, vvi-miR171c, vvi-miR171d, vvi-miR171e, vvi-miR171f, vvi-miR171g, vvi-miR171h, vvi-miR171i, vvi-miR171j, vvi-miR172a, vvi-miR172b, vvi-miR172c, vvi-miR172d, vvi-miR2111-3p, vvi-miR2111-5p, vvi-miR2950-3p, vvi-miR2950-5p, vvi-miR319b, vvi-miR319c, vvi-miR319e, vvi-miR319f, vvi-miR319g, vvi-miR3623-3p, vvi-miR3623-5p, vvi-miR3624-3p, vvi-miR3624-5p, vvi-miR3625-3p, vvi-miR3625-5p, vvi-miR3626-3p, vvi-miR3626-5p, vvi-miR3627-3p, vvi-miR3627-5p, vvi-miR3628-3p, vvi-miR3628-5p, vvi-miR3629a-3p, vvi-miR3629a-5p, vvi-miR3629b, vvi-miR3629c, vvi-miR3630-3p, vvi-miR3630-5p, vvi-miR3631a-3p, vvi-miR3631a-5p, vvi-miR3631b-3p, vvi-miR3631b-5p, vvi-miR3631c, vvi-miR3631d, vvi-miR3632-3p, vvi-miR3632-5p, vvi-miR3633a-3p, vvi-miR3633a-5p, vvi-miR3633b-3p, vvi-miR3633b-5p, vvi-miR3634-3p, vvi-miR3634-5p, vvi-miR3635-3p, vvi-miR3635-5p, vvi-miR3636-3p, vvi-miR3636-5p, vvi-miR3637-3p, vvi-miR3637-5p, vvi-miR3638-3p, vvi-miR3638-5p, vvi-miR3639-3p, vvi-miR3639-5p, vvi-miR3640-3p, vvi-miR3640-5p, vvi-miR390, vvi-miR393a, vvi-miR393b, vvi-miR394a, vvi-miR394b, vvi-miR394c, vvi-miR395a, vvi-miR395b, vvi-miR395c, vvi-miR395d, vvi-miR395e, vvi-miR395f, vvi-miR395g, vvi-miR395h, vvi-miR395i, vvi-miR395j, vvi-miR395k, vvi-miR3951, vvi-miR395m, vvi-miR395n, vvi-miR396a, vvi-miR396b, vvi-miR396c, vvi-miR396d, vvi-miR397a, vvi-miR398a, vvi-miR398b, vvi-miR398c, vvi-miR399a, vvi-miR399b, vvi-miR399c, vvi-miR399d, vvi-miR399e, vvi-miR399f, vvi-miR399g, vvi-miR399h, vvi-miR399i, vvi-miR403a, vvi-miR403b, vvi-miR403c, vvi-miR403d, vvi-miR403e, vvi-miR403f, vvi-miR408, vvi-miR477a, vvi-miR477b-3p, vvi-miR477b-5p, vvi-miR479, vvi-miR482, vvi-miR535a, vvi-miR535b, vvi-miR535c, vvi-miR828a, vvi-miR845a, vvi-miR845b, vvi-miR845c, vvi-miR845d, vvi-miR845e, xbo-miR-124, xbo-miR-137, xbo-miR-2012, xbo-miR-2013, xbo-miR-219, xbo-miR-4907, xbo-miR-92a, xbo-miR-92b, xla-let-7a-3p, xla-let-7a-5p, xla-let-7b-3p, xla-let-7b-5p, xla-let-7c-3p, xla-let-7c-5p, xla-let-7e-3p, xla-let-7e-5p, xla-let-7f-3p, xla-let-7f-5p, xla-let-7i-3p, xla-let-7i-5p, xla-let-7j-3p, xla-let-7j-5p, xla-miR-100-2-3p, xla-miR-100-3p, xla-miR-100-5p, xla-miR-101-3p, xla-miR-101-5p, xla-miR-103a-3p, xla-miR-103a-5p, xla-miR-103b-3p, xla-miR-103b-5p, xla-miR-106-3p, xla-miR-106-5p, xla-miR-107-3p, xla-miR-107-5p, xla-miR-10a-3p, xla-miR-10a-5p, xla-miR-10b-3p, xla-miR-10b-5p, xla-miR-122-3p, xla-miR-122-5p, xla-miR-124-2-5p, xla-miR-124-3p, xla-miR-124-5p, xla-miR-125a-3p, xla-miR-125a-5p, xla-miR-125b-3p, xla-miR-125b-5p, xla-miR-126-2-3p, xla-miR-126-3p, xla-miR-126-5p, xla-miR-128-2-5p, xla-miR-128-3p, xla-miR-128-5p, xla-miR-1306, xla-miR-130a-3p, xla-miR-130a-5p, xla-miR-130b-3p, xla-miR-130b-5p, xla-miR-130c-3p, xla-miR-130c-5p, xla-miR-133a, xla-miR-133b, xla-miR-133d, xla-miR-135-3p, xla-miR-135-5p, xla-miR-138-2-3p, xla-miR-138-3p, xla-miR-138-5p, xla-miR-140-3p, xla-miR-140-5p, xla-miR-142, xla-miR-142-3p, xla-miR-142-5p, xla-miR-143-3p, xla-miR-143-5p, xla-miR-144-3p, xla-miR-144-5p, xla-miR-145-3p, xla-miR-145-5p, xla-miR-146a-3p, xla-miR-146a-5p, xla-miR-146b-3p, xla-miR-146b-5p, xla-miR-148a-3p, xla-miR-148a-5p, xla-miR-148b-3p, xla-miR-148b-5p, xla-miR-15a-3p, xla-miR-15a-5p, xla-miR-15b-3p, xla-miR-15b-5p, xla-miR-15c, xla-miR-15c-3p, xla-miR-15c-5p, xla-miR-16a-3p, xla-miR-16a-5p, xla-miR-16b-3p, xla-miR-16b-5p, xla-miR-16c-3p, xla-miR-16c-5p, xla-miR-17-2-3p, xla-miR-17-3p, xla-miR-17-5p, xla-miR-181a-3p, xla-miR-181a-5p, xla-miR-181b-2-3p, xla-miR-181b-3p, xla-miR-181b-5p, xla-miR-182-2-3p, xla-miR-182-3p, xla-miR-182-5p, xla-miR-183-3p, xla-miR-183-5p, xla-miR-184-3p, xla-miR-184-5p, xla-miR-18a-3p, xla-miR-18a-5p, xla-miR-18b-3p, xla-miR-18b-5p, xla-miR-190-3p, xla-miR-190-5p, xla-miR-191-3p, xla-miR-191-5p, xla-miR-192-3p, xla-miR-192-5p, xla-miR-193-3p, xla-miR-193-5p, xla-miR-194, xla-miR-194-3p, xla-miR-194-5p, xla-miR-199-3p, xla-miR-199-5p, xla-miR-19a-3p, xla-miR-19a-5p, xla-miR-19b, xla-miR-19b-3p, xla-miR-19b-5p, xla-miR-1a-3p, xla-miR-1a-5p, xla-miR-1b, xla-miR-20, xla-miR-200a-3p, xla-miR-200a-5p, xla-miR-200b-3p, xla-miR-200b-5p, xla-miR-202-3p, xla-miR-202-5p, xla-miR-203-3p, xla-miR-203-5p, xla-miR-205, xla-miR-206-3p, xla-miR-206-5p, xla-miR-20a-3p, xla-miR-20a-5p, xla-miR-210-3p, xla-miR-210-5p, xla-miR-212-3p, xla-miR-212-5p, xla-miR-214-3p, xla-miR-214-5p, xla-miR-216-3p, xla-miR-216-5p, xla-miR-217-3p, xla-miR-217-5p, xla-miR-22-3p, xla-miR-22-5p, xla-miR-221-3p, xla-miR-221-5p, xla-miR-222-3p, xla-miR-222-5p, xla-miR-223, xla-miR-23a, xla-miR-23a-3p, xla-miR-23a-5p, xla-miR-23b-3p, xla-miR-23b-5p, xla-miR-24a-3p, xla-miR-24a-5p, xla-miR-24b-3p, xla-miR-24b-5p, xla-miR-25-3p, xla-miR-25-5p, xla-miR-26-2-3p, xla-miR-26-3p, xla-miR-26-5p, xla-miR-27a-3p, xla-miR-27a-5p, xla-miR-27b-3p, xla-miR-27b-5p, xla-miR-29b-3p, xla-miR-29b-5p, xla-miR-29c-3p, xla-miR-29c-5p, xla-miR-301-3p, xla-miR-301-5p, xla-miR-30a-3p, xla-miR-30a-5p, xla-miR-30b-3p, xla-miR-30b-5p, xla-miR-30c-3p, xla-miR-30c-5p, xla-miR-30d-3p, xla-miR-30d-5p, xla-miR-30e-3p, xla-miR-30e-5p, xla-miR-338-3p, xla-miR-338-5p, xla-miR-33a-3p, xla-miR-33a-5p, xla-miR-33b-3p, xla-miR-33b-5p, xla-miR-34a-3p, xla-miR-34a-5p, xla-miR-34b-3p, xla-miR-34b-5p, xla-miR-363, xla-miR-363-3p, xla-miR-363-5p, xla-miR-375-3p, xla-miR-375-5p, xla-miR-425-2-3p, xla-miR-425-3p, xla-miR-425-5p, xla-miR-427, xla-miR-427-3p, xla-miR-427-5p, xla-miR-428, xla-miR-428a-3p, xla-miR-428a-5p, xla-miR-428b-3p, xla-miR-428b-5p, xla-miR-429, xla-miR-429-3p, xla-miR-429-5p, xla-miR-449-3p, xla-miR-449-5p, xla-miR-455-3p, xla-miR-455-5p, xla-miR-499-3p, xla-miR-499-5p, xla-miR-703, xla-miR-92a-3p, xla-miR-92a-4-5p, xla-miR-92a-5p, xla-miR-92b-3p, xla-miR-92b-5p, xla-miR-93-3p, xla-miR-93-5p, xla-miR-96-3p, xla-miR-96-5p, xla-miR-98-3p, xla-miR-98-5p, xla-miR-99-3p, xla-miR-99-5p, xtr-let-7a, xtr-let-7b, xtr-let-7c, xtr-let-7e, xtr-let-7f, xtr-let-7g, xtr-let-7i, xtr-miR-100, xtr-miR-101a, xtr-miR-103, xtr-miR-106, xtr-miR-107, xtr-miR-10a, xtr-miR-10b, xtr-miR-10c, xtr-miR-122, xtr-miR-124, xtr-miR-125a, xtr-miR-125b, xtr-miR-126-3p, xtr-miR-126-5p, xtr-miR-128, xtr-miR-129, xtr-miR-130a, xtr-miR-130b, xtr-miR-130c, xtr-miR-132, xtr-miR-133a, xtr-miR-133b, xtr-miR-133c, xtr-miR-133d, xtr-miR-135, xtr-miR-137, xtr-miR-138, xtr-miR-139, xtr-miR-140, xtr-miR-142-3p, xtr-miR-142-5p, xtr-miR-143, xtr-miR-144, xtr-miR-145, xtr-miR-146a, xtr-miR-146b, xtr-miR-148a, xtr-miR-148b, xtr-miR-150, xtr-miR-153, xtr-miR-155, xtr-miR-15a, xtr-miR-15b, xtr-miR-15c, xtr-miR-16a, xtr-miR-16b, xtr-miR-16c, xtr-miR-17-3p, xtr-miR-17-5p, xtr-miR-181a-1-3p, xtr-miR-181a-2-3p, xtr-miR-181a-5p, xtr-miR-181b, xtr-miR-182-3p, xtr-miR-182-5p, xtr-miR-183, xtr-miR-184, xtr-miR-187, xtr-miR-18a-3p, xtr-miR-18a-5p, xtr-miR-18b, xtr-miR-190, xtr-miR-191, xtr-miR-192, xtr-miR-193, xtr-miR-194, xtr-miR-196a, xtr-miR-196b, xtr-miR-199a-3p, xtr-miR-199a-5p, xtr-miR-199b, xtr-miR-19a, xtr-miR-19b, xtr-miR-1a, xtr-miR-1b, xtr-miR-200a, xtr-miR-200b, xtr-miR-202-3p, xtr-miR-202-5p, xtr-miR-203, xtr-miR-204, xtr-miR-205a, xtr-miR-205b, xtr-miR-206, xtr-miR-208, xtr-miR-20a-3p, xtr-miR-20a-5p, xtr-miR-20b, xtr-miR-210, xtr-miR-212, xtr-miR-214, xtr-miR-215, xtr-miR-216, xtr-miR-217, xtr-miR-218, xtr-miR-2184, xtr-miR-2188, xtr-miR-219, xtr-miR-22-3p, xtr-miR-22-5p, xtr-miR-221, xtr-miR-222, xtr-miR-223, xtr-miR-23a, xtr-miR-23b, xtr-miR-24a-3p, xtr-miR-24a-5p, xtr-miR-24b, xtr-miR-25, xtr-miR-26, xtr-miR-27a, xtr-miR-27b, xtr-miR-27c, xtr-miR-29a, xtr-miR-29b, xtr-miR-29c-3p, xtr-miR-29c-5p, xtr-miR-29d, xtr-miR-301, xtr-miR-302, xtr-miR-30a-3p, xtr-miR-30a-5p, xtr-miR-30b, xtr-miR-30c, xtr-miR-30d, xtr-miR-30e, xtr-miR-31a, xtr-miR-31b, xtr-miR-320, xtr-miR-338, xtr-miR-33a, xtr-miR-33b, xtr-miR-34a, xtr-miR-34b, xtr-miR-363-3p, xtr-miR-363-5p, xtr-miR-365, xtr-miR-367, xtr-miR-375, xtr-miR-383, xtr-miR-425-5p, xtr-miR-427, xtr-miR-428a, xtr-miR-428b, xtr-miR-429, xtr-miR-449a-3p, xtr-miR-449a-5p, xtr-miR-449b-3p, xtr-miR-449b-5p, xtr-miR-449c-3p, xtr-miR-449c-5p, xtr-miR-451, xtr-miR-454-3p, xtr-miR-455, xtr-miR-460a-3p, xtr-miR-460b-3p, xtr-miR-489, xtr-miR-499, xtr-miR-551b-3p, xtr-miR-7, xtr-miR-9-3p, xtr-miR-9-5p, xtr-miR-92a, xtr-miR-92b, xtr-miR-93a, xtr-miR-93b, xtr-miR-9406, xtr-miR-9407, xtr-miR-96, xtr-miR-98, xtr-miR-99, xtr-miR-9a-3p, xtr-miR-9a-5p, xtr-miR-9b-3p, xtr-miR-9b-5p, zma-miR11969-3p, zma-miR11969-5p, zma-miR11970-3p, zma-miR11970-5p, zma-miR1432-3p, zma-miR1432-5p, zma-miR156a-3p, zma-miR156a-5p, zma-miR156b-3p, zma-miR156b-5p, zma-miR156c, zma-miR156d-3p, zma-miR156d-5p, zma-miR156e-3p, zma-miR156e-5p, zma-miR156f-3p, zma-miR156f-5p, zma-miR156g-3p, zma-miR156g-5p, zma-miR156 h-3p, zma-miR156 h-5p, zma-miR156i-3p, zma-miR156i-5p, zma-miR156j-3p, zma-miR156j-5p, zma-miR156k-3p, zma-miR156k-5p, zma-miR1561-3p, zma-miR1561-5p, zma-miR159a-3p, zma-miR159a-5p, zma-miR159b-3p, zma-miR159b-5p, zma-miR159c-3p, zma-miR159c-5p, zma-miR159d-3p, zma-miR159d-5p, zma-miR159e-3p, zma-miR159e-5p, zma-miR159f-3p, zma-miR159f-5p, zma-miR159g-3p, zma-miR159g-5p, zma-miR159 h-3p, zma-miR159 h-5p, zma-miR159i-3p, zma-miR159i-5p, zma-miR159j-3p, zma-miR159j-5p, zma-miR159k-3p, zma-miR159k-5p, zma-miR160a-3p, zma-miR160a-5p, zma-miR160b-3p, zma-miR160b-5p, zma-miR160c-3p, zma-miR160c-5p, zma-miR160d-3p, zma-miR160d-5p, zma-miR160e, zma-miR160f-3p, zma-miR160f-5p, zma-miR160g-3p, zma-miR160g-5p, zma-miR162-3p, zma-miR162-5p, zma-miR164a-3p, zma-miR164a-5p, zma-miR164b-3p, zma-miR164b-5p, zma-miR164c-3p, zma-miR164c-5p, zma-miR164d-3p, zma-miR164d-5p, zma-miR164e-3p, zma-miR164e-5p, zma-miR164f-3p, zma-miR164f-5p, zma-miR164g-3p, zma-miR164g-5p, zma-miR164 h-3p, zma-miR164 h-5p, zma-miR166a-3p, zma-miR166a-5p, zma-miR166b-3p, zma-miR166b-5p, zma-miR166c-3p, zma-miR166c-5p, zma-miR166d-3p, zma-miR166d-5p, zma-miR166e, zma-miR166f, zma-miR166g-3p, zma-miR166g-5p, zma-miR166 h-3p, zma-miR166 h-5p, zma-miR166i-3p, zma-miR166i-5p, zma-miR166j-3p, zma-miR166j-5p, zma-miR166k-3p, zma-miR166k-5p, zma-miR1661-3p, zma-miR1661-5p, zma-miR166m-3p, zma-miR166m-5p, zma-miR166n-3p, zma-miR166n-5p, zma-miR167a-3p, zma-miR167a-5p, zma-miR167b-3p, zma-miR167b-5p, zma-miR167c-3p, zma-miR167c-5p, zma-miR167d-3p, zma-miR167d-5p, zma-miR167e-3p, zma-miR167e-5p, zma-miR167f-3p, zma-miR167f-5p, zma-miR167g-3p, zma-miR167g-5p, zma-miR167 h-3p, zma-miR167 h-5p, zma-miR167i-3p, zma-miR167i-5p, zma-miR167j-3p, zma-miR167j-5p, zma-miR168a-3p, zma-miR168a-5p, zma-miR168b-3p, zma-miR168b-5p, zma-miR169a-3p, zma-miR169a-5p, zma-miR169b-3p, zma-miR169b-5p, zma-miR169c-3p, zma-miR169c-5p, zma-miR169d, zma-miR169e, zma-miR169f-3p, zma-miR169f-5p, zma-miR169g, zma-miR169h, zma-miR169i-3p, zma-miR169i-5p, zma-miR169j-3p, zma-miR169j-5p, zma-miR169k-3p, zma-miR169k-5p, zma-miR1691-3p, zma-miR1691-5p, zma-miR169m-3p, zma-miR169m-5p, zma-miR169n-3p, zma-miR169n-5p, zma-miR1690-3p, zma-miR1690-5p, zma-miR169p-3p, zma-miR169p-5p, zma-miR169q-3p, zma-miR169q-5p, zma-miR169r-3p, zma-miR169r-5p, zma-miR171a-3p, zma-miR171a-5p, zma-miR171b-3p, zma-miR171b-5p, zma-miR171c-3p, zma-miR171c-5p, zma-miR171d-3p, zma-miR171d-5p, zma-miR171e-3p, zma-miR171e-5p, zma-miR171f-3p, zma-miR171f-5p, zma-miR171g-3p, zma-miR171g-5p, zma-miR171 h-3p, zma-miR171 h-5p, zma-miR171i-3p, zma-miR171i-5p, zma-miR171j-3p, zma-miR171j-5p, zma-miR171k-3p, zma-miR171k-5p, zma-miR1711-3p, zma-miR1711-5p, zma-miR171m-3p, zma-miR171m-5p, zma-miR171n-3p, zma-miR171n-5p, zma-miR172a, zma-miR172b-3p, zma-miR172b-5p, zma-miR172c-3p, zma-miR172c-5p, zma-miR172d-3p, zma-miR172d-5p, zma-miR172e, zma-miR2118a, zma-miR2118b, zma-miR2118c, zma-miR2118d, zma-miR2118e, zma-miR2118f, zma-miR2118g, zma-miR2275a-3p, zma-miR2275a-5p, zma-miR2275b-3p, zma-miR2275b-5p, zma-miR2275c-3p, zma-miR2275c-5p, zma-miR2275d-3p, zma-miR2275d-5p, zma-miR319a-3p, zma-miR319a-5p, zma-miR319b-3p, zma-miR319b-5p, zma-miR319c-3p, zma-miR319c-5p, zma-miR319d-3p, zma-miR319d-5p, zma-miR390a-3p, zma-miR390a-5p, zma-miR390b-3p, zma-miR390b-5p, zma-miR393a-3p, zma-miR393a-5p, zma-miR393b-3p, zma-miR393b-5p, zma-miR393c-3p, zma-miR393c-5p, zma-miR394a-3p, zma-miR394a-5p, zma-miR394b-3p, zma-miR394b-5p, zma-miR395a-3p, zma-miR395a-5p, zma-miR395b-3p, zma-miR395b-5p, zma-miR395c-3p, zma-miR395c-5p, zma-miR395d-3p, zma-miR395d-5p, zma-miR395e-3p, zma-miR395e-5p, zma-miR395f-3p, zma-miR395f-5p, zma-miR395g-3p, zma-miR395g-5p, zma-miR395 h-3p, zma-miR395 h-5p, zma-miR395i-3p, zma-miR395i-5p, zma-miR395j-3p, zma-miR395j-5p, zma-miR395k-3p, zma-miR395k-5p, zma-miR3951-3p, zma-miR3951-5p, zma-miR395m-3p, zma-miR395m-5p, zma-miR395n-3p, zma-miR395n-5p, zma-miR3950-3p, zma-miR3950-5p, zma-miR395p-3p, zma-miR395p-5p, zma-miR396a-3p, zma-miR396a-5p, zma-miR396b-3p, zma-miR396b-5p, zma-miR396c, zma-miR396d, zma-miR396e-3p, zma-miR396e-5p, zma-miR396f-3p, zma-miR396f-5p, zma-miR396g-3p, zma-miR396g-5p, zma-miR396h, zma-miR397a-3p, zma-miR397a-5p, zma-miR397b-3p, zma-miR397b-5p, zma-miR398a-3p, zma-miR398a-5p, zma-miR398b-3p, zma-miR398b-5p, zma-miR399a-3p, zma-miR399a-5p, zma-miR399b-3p, zma-miR399b-5p, zma-miR399c-3p, zma-miR399c-5p, zma-miR399d-3p, zma-miR399d-5p, zma-miR399e-3p, zma-miR399e-5p, zma-miR399f-3p, zma-miR399f-5p, zma-miR399g-3p, zma-miR399g-5p, zma-miR399 h-3p, zma-miR399 h-5p, zma-miR399i-3p, zma-miR399i-5p, zma-miR399j-3p, zma-miR399j-5p, zma-miR408a, zma-miR408b-3p, zma-miR408b-5p, zma-miR444a, zma-miR444b, zma-miR482-3p, zma-miR482-5p, zma-miR528a-3p, zma-miR528a-5p, zma-miR528b-3p, zma-miR528b-5p, zma-miR529-3p, zma-miR529-5p, zma-miR827-3p, zma-miR827-5p, or a combination thereof.

Many miRNAs exhibit a tissue-specific expression pattern. Examples of miRNAs that are ES-cell specific include miR-296. Examples of miRNAs that are expressed in ES cells, but upregulated on differentiation include miR-21 and miR-22. Examples of miRNAs that are expressed in both ES cells and various adult tissues include miR-15a, miR-16, miR-19b, miR-92, miR-93, miR-96, miR-130 and miR-130b. Examples of miRNAs that are enriched during mouse brain development include miR-128, miR-19b, miR-9, miR-125b, miR-131, miR-178, miR-124a, miR-266 and miR-103. Examples of miRNAs that are enriched in adult brain include miR-9*, miR-125a, miR-125b, miR-128, miR-132, miR-137, miR-139, miR-7, miR-9, miR124a, miR-124b, miR-135, miR-153, miR-149, miR-183, miR-190, and miR-219. Examples of miRNAs that are enriched in lung include miR-18, miR-19a, miR-24, miR-32, miR-130, miR-213, miR-20, miR-141, miR-193 and miR-200b. Examples of miRNAs that are enriched in spleen include miR99a, miR-127, miR-142-a, miR-142-s, miR-151, miR-189b and miR-212. Examples of miRNAs that are hematopoietic tissues include miR-181, miR-223 and miR-142. Examples of miRNAs that are enriched in liver include miR-122a, miR-152, miR-194, miR-199 and miR-215. Examples of miRNAs that are enriched in heart include miR-1b, miR-1d, miR-133, miR-206, miR-208, and miR-143. Examples of miRNAs that are enriched in kidney include miR-30b, miR-30c, miR-18, miR-20, miR-24, miR-32, miR-141, miR-193 and miR-200b. Examples of miRNAs that are ubiquitously expressed include miR-16, miR-26a, miR-27a, miR-143a, miR-21, let-7a, miR-7b, miR-30b and miR-30c. See, Houbaviy et al, Dev. Cell (2003) 5:351-358; Sempere et al, Genome Biol. (2004) 5, R13; and Krichevsky et al, RNA (2003), 9:1274-1281, each of which is incorporated herein by reference in its entirety.

Some miRNAs can be abnormally expressed in a diseased state (e.g., tumorigenesis). For example, miR-15 and miR-16 are downregulated in chronic lymphocytic leukemia; miR-26a and miR-99a are downregulated in lung cancer cell lines; miR143/miR-145 cluster are downregulated in colon cancers; and miR-155 is upregulated in Burkitt Lymphoma, and ES cells, embryonic stem cells. See, Calin et al, Proc Natl Acad Sci (2002) 99:15524-15529; Calin et al, Proc Natl Acad Sci (2004) 101:2999-3004; Michael et al, Mol Cancer Res (2003) 1:882-891, and Metzier et al, Genes Chromosomes Cancer (2004) 39:167-169, each of which is incorporated herein by reference in its entirety.

### Target Polypeptides

In various embodiments, miRNA-dependent polynucleotide circuits described herein include a nucleotide sequence encoding a target polypeptide.

In some embodiments, the target polypeptide is a payload polypeptide.

In some embodiments, the payload polypeptide is a gene editing nuclease, a chimeric antigen receptor (CAR), an antibody, a cytotoxic protein, a cell surface receptor, a transcription factor, an enzyme, a reporter protein, a cytokine, or a polypeptide that confers drug resistance.

In some embodiments, the payload polypeptide is a gene editing nuclease. Examples of gene editing nucleases include, but are not limited to, a CRISPR-associated protein (Cas) nucleases (e.g., Cas9), zinc finger nucleases (ZFNs), transcription activator-like effector nucleases (TALENs), meganucleases, other endo- or exo-nucleases, and variants or fragments thereof. In one embodiment, the payload polypeptide is a Cas9 nuclease.

In some embodiments, the payload polypeptide is a chimeric antigen receptor (CAR). CARs typically consist of four components: i) an extracellular antigen recognition domain, most commonly a single chain variable fragment (scFv), ii) structural components, such as hinge and transmembrane domains, iii) a costimulatory domain that provides signals to sustain CAR T cell effector functions, and iv) a CD3 activation domain.

In some embodiments, the payload polypeptide is a cytotoxic protein. A cytotoxic protein may induce cell death in when expressed in a host cell and/or cell death of a neighboring cell when secreted by the host cell. In some embodiments, the cytotoxic protein is a caspase, p53, diphtheria toxin (DT), Pseudomonas Exotoxin A (PEA), Type I ribosome inactivating proteins (RIPs) (e.g., saporin and gelonin), Type II RIPs (e.g., ricin), Shiga-like toxin 1 (Slt1), photosensitive reactive oxygen species (e.g., killer-red).

In some embodiments, the payload polypeptide is a reporter protein. A reporter protein may produce a detectable signal which allows detection of the target cell or tissue. Examples of reporter proteins include, without limitation, b-lactamase, b-galactosidase (LacZ), alkaline phosphatase, thymidine kinase, green fluorescent protein (GFP), red fluorescent protein (RFP), yellow fluorescent protein (YFP), chloramphenicol acetyltransferase (CAT), luciferase, membrane bound proteins including, for example, CD2, CD4, CD8, the influenza hemagglutinin protein, to which high affinity antibodies directed thereto exist or can be produced by conventional means, and fusion proteins comprising a membrane bound protein appropriately fused to an antigen tag domain from, among others, hemagglutinin or Myc.

In some embodiments, the payload polypeptide is an antibody or antigen binding fragment thereof (e.g., antigen-binding fragment of a monoclonal antibody, a single chain variable fragment (scFv), a Fab, a Fab′, a F(ab′) 2, and a Fv fragment). In some embodiments, the antibody or antigen binding fragment thereof specifically binds to a cell surface receptor, such as an immune checkpoint receptor (e.g., PD1, PDL1, and CTLA4) or additional cell surface receptors involved in cell growth and activation (e.g., OX40, CD200R, SIRPα, CSF1R, 4-1BB, CD40, and NKG2D). Examples of therapeutic antibodies that can be expressed by the miRNA-dependent polynucleotide circuits include e.g., a monoclonal antibody such as sutimlimab, naxitamab, margetuximab, tanezumab, narsoplimab, evinacumab, aducanumab, tralokinumab, dostarlimab, teplizumab, omburtamab, inolimomb, ansuvimab, bimekizumab, balstilimab, anifrolumab, and the like.

In some embodiments, the payload polypeptide is a ligand for a cell surface receptor. Exemplary ligands suitable for use as payloads include, but are not limited to, NKG2D ligands, neuropilin ligands, Flt3 ligand, 4-1BBL, CD40L, GITRL, LIGHT, and CD47.

In some embodiments, the payload polypeptide is a soluble receptor. Exemplary soluble receptors suitable for use as payloads include, but are not limited to, soluble receptors such as IL-35R, IL-15R, IL-2R, IL-12R, IL-13R, TGFBR1, TGFBR2, SIRPα, PD-1, interferon receptors, VEGF receptors, TNF-α-binding receptors such as etanercept, and γδ T cell receptors.

In some embodiments, the payload polypeptide is a cytokine. Exemplary cytokines suitable for use as payloads include, but are not limited to, IL-1, IL-12, IL-15, IL-18, IL-36, TNFα, IFNα, IFNβ, and IFNγ.

In some embodiments, the payload molecule is a chemokine. Exemplary chemokines suitable for use as payloads include, but are not limited to, CXCL10, CXCL9, CCL21, CCL4, and CCL5.

In some embodiments, the payload polypeptide is an enzyme. Exemplary enzymes suitable for use as payloads include, but are not limited to, an adenosine deaminase, 15-Hydroxyprostaglandin Dehydrogenase, a matrix metalloprotease (e.g., MMP9), a collagenase, a hyaluronidase, a gelatinase, and an elastase. In some embodiments, the enzyme is part of a gene directed enzyme prodrug therapy (GDEPT) system, such as herpes simplex virus thymidine kinase, cytosine deaminase, nitroreductase, carboxypeptidase G2, purine nucleoside phosphorylase, or cytochrome P450.

Other suitable payload polypeptides include, but are not limited to, an angiogenic agent (e.g., vascular endothelial growth factor (VEGF); an anti-angiogenic agent (e.g., a soluble VEGF receptor); a blood factor (e.g., Activase® (alteplase) tissue plasminogen activator, coagulation Factor IX, b-globin hemoglobin, Factor V (FV), FVII, FVIII, FIX, FX, FXI, FXIII, FII, Protein C, C1-inhibitor, prekallikrein, high molecular weight kininogen (HMWK) or von Willebrand's factor, and the like); a chemokine (e.g., IP-10, Mig, Groa/IL-8, RANTES, MIR-Ia, MIR-Ib, MCP-1, PF-4, and the like); a cytokine; a colony stimulating factor (e.g., granulocyte colony stimulating factor (G-CSF), granulocyte-monocyte colony stimulating factor, macrophage colony stimulating factor, megakaryocyte colony stimulating factor and the like); an enzyme (e.g., a-glucosidase, alglucerase), an enzyme activator (e.g., tissue plasminogen activator); an erythropoietin (“EPO”, e.g., epoetin-a, darbepoietin-a, epoetin-b, and the like); a growth hormone (e.g., a somatotropin, a human growth hormone and the like); a growth factor (e.g. beclapermin PDGF, trafermin bFGF, ancestim stem cell factor, keratinocyte growth factor, an acidic fibroblast growth factor, a stem cell factor, a basic fibroblast growth factor, a hepatocyte growth factor, and the like); an insulin; an interferon (e.g., IFN-g, IFN-a, IFN-b, IFN-w, IFN-x); an interleukin (e.g., IL-1, IL-2, including, e.g., Proleukin®, IL-3, IL-4, IL-5, IL-6, IL-7, IL-8, IL-9, etc.); a cell surface receptor; a protein vaccine; a neuroactive peptide (e.g., bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, etc.); other proteins such as a thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, an insulin-like growth factor, a luteinizing hormone, a macrophage activating factor, tumor necrosis factor, a neutrophil chemotactic factor, a nerve growth factor a tissue inhibitor of metalloproteinases; a vasoactive intestinal peptide, angiogenin, angiotropin, fibrin, hirudin, a leukemia inhibitory factor, an IL-1 receptor antagonist (e.g., anakinra), an ion channel, e.g., cystic fibrosis transmembrane conductance regulator (CFTR), dystrophin, utrophin, a tumor suppressor, lysosomal enzyme acid a-glucosidase (GAA) and the like, or any functional fragments or variants thereof.

In some embodiments, the target polypeptide is a gene of the vector carrying the miRNA-dependent polynucleotide circuit. For example, the miRNA-dependent polynucleotide circuit may be used to modulate expression of a viral protein encoded by a viral vector.

### Vectors

In another aspect, provided herein is a vector comprising a miRNA-dependent polynucleotide circuit described herein.

In various embodiments, the vector described herein is a viral vector. In some embodiments, the viral vector is a retroviral vector (e.g., lentiviral vector), an adenovirus vector (e.g., Ad2, Ad5, Ad7 or Ad12), a parvovirus vector (e.g., adeno-associated virus), an influenza virus vector, a herpes virus vector, a poxvirus vector, a rhabdovirus vector (e.g., vesicular stomatitis virus (VSV)), a paramyxovirus vector (e.g., Sendai virus (SeV)), a morbillivirus vector (e.g., Measles virus, Rabies virus), a reovirus vector, a vaccinia virus vector, a baculoviral vector, or a SV-40-based viral vector.

In one embodiment, the vector described herein is a lentiviral vector.

In one embodiment, the vector described herein is a Sendai virus (SeV) vector.

In some embodiments, when a Sendai virus (SeV) vector is used, the nucleotide sequence encoding an RNA binding protein described herein (e.g., a ribosomal frameshift stimulator, an iron regulatory protein, Lin28, MS2 bacteriophage coat protein, PP7 bacteriophage coat protein, and U1 small nuclear ribonucleoprotein A (U1A)), along with one or more microRNA (miRNA) Silencing Element (MSE) sequences, may be positioned in any insertion site in the SeV genome, for example, between N and P, between P and M, between M and F, between F and HN, or between HN and L.

In some embodiments, when a Sendai virus (SeV) vector is used, the nucleotide sequence encoding a target polypeptide, along with an RNA element to which an RNA-binding protein specifically binds (e.g., a ribosomal frameshift element, an iron response element, a pre-let-7 element, an MS2 responsive RNA element, a PP7 responsive RNA element, and a U1A responsive RNA element), may be positioned in any insertion site in the SeV genome, for example, between N and P, between P and M, between M and F, between F and HN, or between HN and L.

In some embodiments, when a Sendai virus (SeV) vector is used, the nucleotide sequence encoding an RNA binding protein (e.g., a ribosomal frameshift stimulator, an iron regulatory protein, Lin28, MS2 bacteriophage coat protein, PP7 bacteriophage coat protein, and U1 small nuclear ribonucleoprotein A (U1A)), along with one or more microRNA (miRNA) Silencing Element (MSE) sequences, is positioned 5′ to the nucleotide sequence encoding a target polypeptide, along with an RNA element to which the RNA-binding protein specifically binds (e.g., a ribosomal frameshift element, an iron response element, a pre-let-7 element, an MS2 responsive RNA element, a PP7 responsive RNA element, and a U1A responsive RNA element) in the SeV genome.

In some embodiments, when a Sendai virus (SeV) vector is used, the nucleotide sequence encoding an RNA binding protein (e.g., a ribosomal frameshift stimulator, an iron regulatory protein, Lin28, MS2 bacteriophage coat protein, PP7 bacteriophage coat protein, and U1 small nuclear ribonucleoprotein A (U1A)), along with one or more microRNA (miRNA) Silencing Element (MSE) sequences, is positioned 3′ to the nucleotide sequence encoding a target polypeptide, along with an RNA element to which the RNA-binding protein specifically binds (e.g., a ribosomal frameshift element, an iron response element, a pre-let-7 element, an MS2 responsive RNA element, a PP7 responsive RNA element, and a U1A responsive RNA element) in the SeV genome.

In some embodiments, when a Sendai virus (SeV) vector is used, the nucleotide sequence encoding an RNA binding protein (e.g., a ribosomal frameshift stimulator, an iron regulatory protein, Lin28, MS2 bacteriophage coat protein, PP7 bacteriophage coat protein, and U1 small nuclear ribonucleoprotein A (U1A)), along with one or more microRNA (miRNA) Silencing Element (MSE) sequences, is positioned in between P and M in the SeV genome.

In some embodiments, when a Sendai virus (SeV) vector is used, the nucleotide sequence encoding a target polypeptide, along with an RNA element to which the RNA-binding protein specifically binds (e.g., a ribosomal frameshift element, an iron response element, a pre-let-7 element, an MS2 responsive RNA element, a PP7 responsive RNA element, and a U1A responsive RNA element), is positioned in between N and P in the SeV genome.

In some embodiments, when a Sendai virus (SeV) vector is used, the nucleotide sequence encoding a target polypeptide, along with a pre-let-7 element, is positioned in between N and P in the SeV genome.

In some embodiments, when a Sendai virus (SeV) vector is used, the nucleotide sequence encoding Lin28, along with one or more microRNA (miRNA) Silencing Element (MSE) sequences, is positioned in between P and M in the SeV genome.

In one embodiment, the vector described herein is an adeno-associated virus (AAV) vector. In some embodiments the AAV vector is selected from, without limitation, AAV1, AAV2, AAV3, AAV3B, AAV4, AAV5, AAV6, AAV6.2, AAV7, AAV8, AAV9, AAV10, AAV11, AAV type hu11 (AAV hu11), AAV12, AAV13, AAVDJ, AAV retro (AAV retro), Anc80L65, AAVLK03, AAV type rh32.33 (AAVrh.32.33), AAV PHP.S, AAV PHP.B, AAV PHP.eB, AAVrh.64R1, AAVhu.37, AAVrh.8, and AAV2/8,AAV2G9. In some embodiments, the AAV vector is AAV8 vector.

In some embodiments, the vector is a non-viral vector. In some embodiments, the non-viral vector is a transposon such as, but are not limited to, a PiggyBac transposon and a sleeping beauty transposon. In some embodiments, the vector is a plasmid.

In one embodiment, the vector described herein is a bacterial vector. For example, the bacterial vector may be a Salmonella, Lactococcus, or Listeria vector.

In one embodiment, the vector described herein is a phage vector.

The vectors used herein may naturally or be genetically engineered to target gene delivery to specific cell types or tissues. In some embodiments, the viral vectors described herein may have natural or genetically engineered envelope proteins that interact with cell-type-specific receptors. In some embodiments, the expression of the payload polypeptide in specific tissue or cell types by using tissue or cell type-specific promoters. Promoters that are active in certain tissues or cell types can be used to further regulate the expression of the payload polypeptide described herein.

As used herein, the term “isolated” means that the referenced material (e.g., a cell or virus) is removed from its native environment. Thus, an isolated biological material can be free of some or all cellular components, i.e., components of the cells in which the native material occurs naturally (e.g., cytoplasmic or membrane component). A material shall be deemed isolated if it is present in a cell extract or supernatant. In the case of nucleic acid molecules, an isolated nucleic acid includes, without limitation, a PCR product, an isolated RNA (e.g., mRNA or miRNA), a DNA (e.g., cDNA), or a restriction fragment. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified virus is preferably substantially free of host cell or culture components, including tissue culture or egg proteins, non-specific pathogens, and the like. As used herein, the term “substantially free” is used operationally, in the context of analytical testing of the material. Preferably, purified material substantially free of contaminants is at least 50% pure; more preferably, at least 90% pure, and still more preferably at least 99% pure. Purity can be evaluated by chromatography, gel electrophoresis, immunoassay, composition analysis, biological assay, and other methods known in the art.

Methods for purification are well-known in the art. Viral particles can be purified by ultrafiltration through sucrose cushions or by ultracentrifugation, preferably continuous centrifugation (see Furminger, In: Nicholson, Webster and May (eds.), Textbook of Influenza, Chapter 24, pp. 324-332). Other purification methods are possible and contemplated herein. A purified material may contain less than about 50%, preferably less than about 75%, and most preferably less than about 90%, of the cellular components, media, proteins, or other undesirable components or impurities (as context requires), with which it was originally associated. The term “substantially pure” indicates the highest degree of purity which can be achieved using conventional purification techniques known in the art.

### Compositions

In one aspect, provided herein is a composition comprising a miRNA-dependent polynucleotide circuit described herein or a vector thereof, and a carrier or excipient. The composition may be used for introducing the miRNA-dependent polynucleotide circuit described herein to any multicellular organism, including animals, plants and fungi.

In some embodiments, the composition is a pharmaceutical composition comprising a miRNA-dependent polynucleotide circuit described herein or a vector thereof, and a pharmaceutically acceptable carrier or excipient. The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilizing agent(s), and other carrier agents that may aid or increase the viral entry into the target site (such as for example a lipid delivery system).

The compound described herein can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

In some embodiments, two or more combinations of the compounds of the disclosure may be administered to the subject. The compound(s) may also be administered in combination with one or more additional therapeutic agents. This combination can allow separate, continuous or simultaneous administration with the other active ingredients of the above compounds. This combination may be provided in the form of a pharmaceutical composition.

As used herein, the term “combination” is used by the combination agents as defined above dependently or independently, or by the use of different fixed combinations with different amounts of combination agents, i.e. simultaneously or at different times. The combination agents can then be administered, for example, simultaneously or staggered in time (i.e., at different times and at equal or different time intervals for any part of the kit). The ratio of the total amount of combination agents administered in a combination can vary, e.g., to address the needs of a subpopulation of patients to be treated or the needs of a single patient, and different needs are the age of the patient, it can be due to gender, weight, etc.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

In some embodiments, the composition further comprises a targeting agent or a carrier that promotes the delivery of the polynucleotide or vector to a target cell or tissue. Exemplary carriers include liposomes, micelles, nanodisperse albumin and its modifications, polymer nanoparticles, dendrimers, inorganic nanoparticles of different compositions.

The appropriate formulation for the compound of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The formulation may also include suitable excipients, such as antioxidants. Examples of antioxidants include phenolic compounds such as BHT or Vitamin E, reducing agents such as methionine or sulfites, and metal chelating agents such as EDTA.

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavoring or coloring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

In some embodiments, the compounds or pharmaceutically acceptable salts thereof described herein can be prepared in parenteral dosage forms such as those suitable for, e.g., intravascular (intravenous or intraarterial), intraperitoneal, intratumoral, intraventricular, intrapleural or intramuscular administration delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol and). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. Suitable pharmaceutical forms for injection can be delivered by any suitable route, including intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. A dosage unit form may contain a predetermined amount of active substance calculated to produce a desired therapeutic effect described herein. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 μg to about 2000 mg. Expressed in proportion, the active compound may be present in a carrier of about 0.25 μg to about 2000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the inhibitor of endocytosis. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the inhibitor of endocytosis with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Host Cells

In one aspect, provided herein is a host cell comprising a polynucleotide or a vector of the present disclosure. The host cell can be either prokaryotic or eukaryotic. Bacterial cells may be preferred prokaryotic host cells in some circumstances and typically are a strain of E. coli such as, for example, the E. coli strains DH5 and RR1. Non-limiting examples of eukaryotic host cells include yeast, insect, and mammalian cells (e.g., from a mouse, rat, monkey, or human cell lines). Non-limiting examples of yeast host cells include, e.g., YPH499, YPH500, and YPH501. Non-limiting examples of mammalian host cells include Chinese hamster ovary (CHO) cells, NIH Swiss mouse embryo cells NIH/3T3, monkey kidney-derived COS-1 cells, and 293 cells which are human embryonic kidney cells. Examples of insect cells include Sf9 cells, which can be transfected with baculovirus expression vectors.

Transformation of appropriate cell hosts with a polynucleotide construct of the present disclosure can accomplished by well-known methods that typically depend on the type of vector used. Successfully transformed cells, i.e. cells that contain a polynucleotide construct of the present disclosure, can be identified by, for example, PCR. Alternatively, the presence of the protein in the supernatant can be detected using antibodies.

### Methods for Selective Expression of Payload Polypeptides

In one aspect, provided herein is a method of selectively expressing a payload polypeptide in a target cell type or tissue or a cell in a specific state within a multicellular organism, comprising introducing to the subject an effective amount of the polynucleotide, or the vector, or the composition (e.g., pharmaceutical composition) of the present disclosure.

The miRNA-dependent polynucleotide circuits may be administered to a subject or used to produce a transgenic fungus, plant or non-human animal. The term “administered” includes delivery by viral or non-viral techniques. Viral delivery mechanisms include but are not limited to adenoviral vectors, adeno-associated viral (AAV) vectors, herpes viral vectors, retroviral vectors (e.g., lentiviral vectors), and baculoviral vectors etc. as described above. Non-viral delivery mechanisms include lipid mediated transfection, liposomes, immunoliposomes, lipofectin, cationic facial amphiphiles (CFAs) and combinations thereof.

The delivery of one or more payloads by a miRNA-dependent polynucleotide circuit according to the invention may be used alone or in combination with other treatments or components of the treatment. Diseases which may be treated include, but are not limited to: cancer, neurological diseases, inherited diseases, heart disease, stroke, arthritis, viral infections and diseases of the immune system.

### EXAMPLES

The following examples are provided to further describe some of the embodiments disclosed herein. The examples are intended to illustrate, not to limit, the disclosed embodiments.

### Example 1. Bicistronic RNA Encoding Epitope

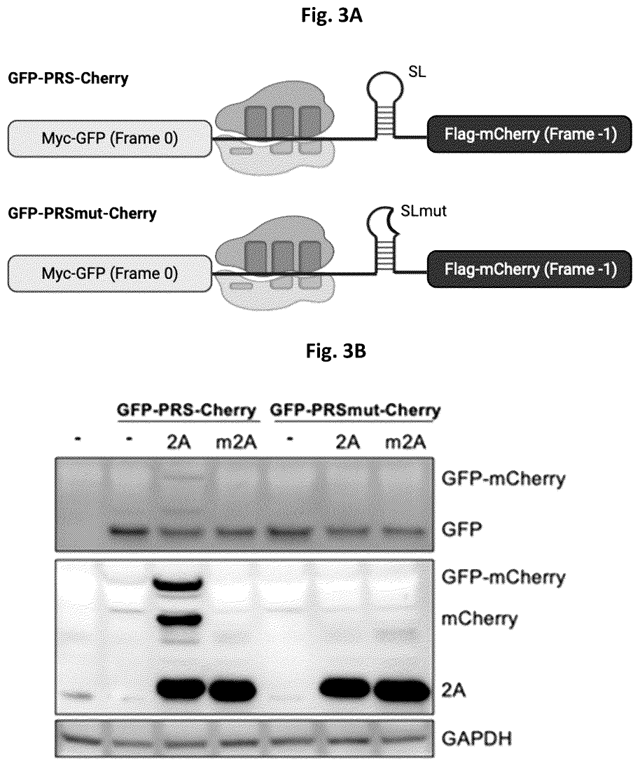

To generate a miRNA-dependent RNA-based program, the ribosomal frameshift motif of EMCV, a member of the cardiovirus family, was incorporated between two genes, one encoding a Myc-epitope tagged green fluorescent protein (Myc-GFP) and the other encoding a Flag-epitope tagged mCherry protein (Flag mCherry) (FIG. 3A). The mCherry transcript was encoded in this element in a-1 reading frame relative to Myc-GFP. However, the addition of the ribosomal frameshift element (RFE) was inserted so that the stem loop (SL) would shift the ribosome in the presence of the 2A protein enabling expression of both Myc-GFP and Flag-mCherry (FIG. 3A). Transfection of this construct design (GFP-PRS-Cherry) or one in which the SL was mutated (GFP-PRSmut-Cherry) then allowed ascertaining of the expression of Myc-GFP and/or mCherry in conditions where either wild type 2A protein or a mutated form of it (m2A) was also present (FIG. 3B). Western blot analyses of these various conditions demonstrated that Flag-mCherry expression could only be detected in the presence of the wild type 2A protein and a functional SL element.

### Example 2. Ribosomal Frameshift Element (RFE)-Based miRNA-Dependent Expression Construct

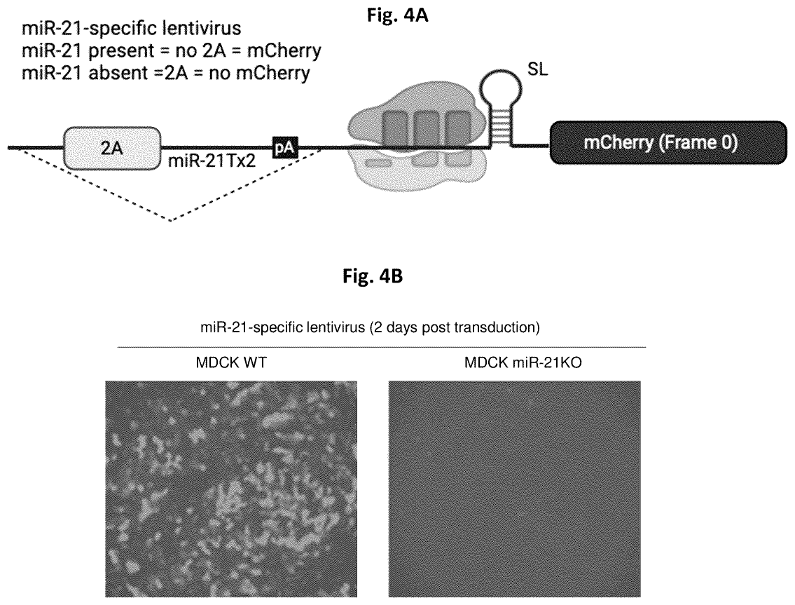

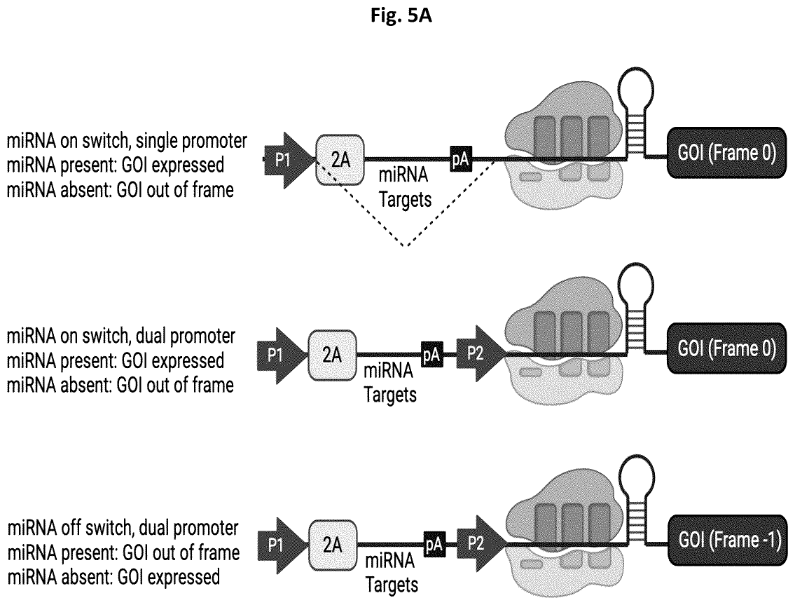

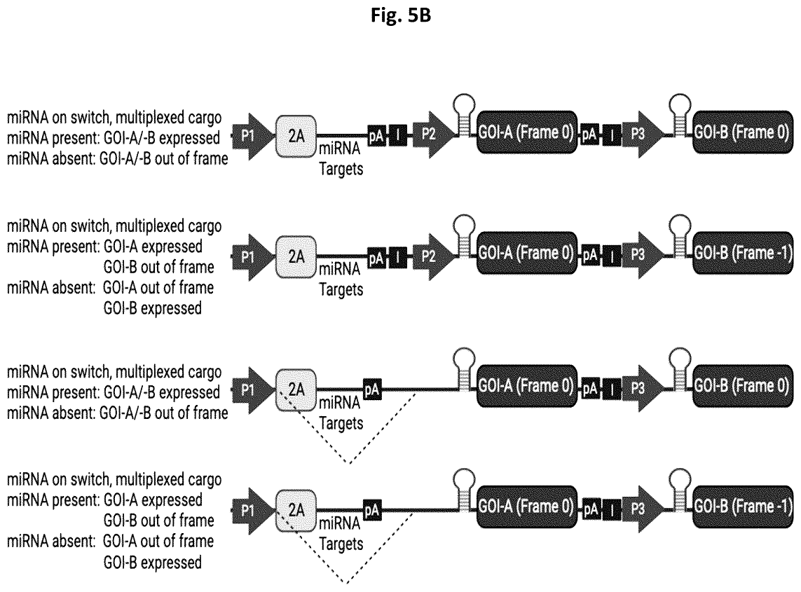

To adapt the aforementioned circuitry to achieve expression only in the presence of a miRNA, a lentivirus-based construct encoding mCherry as the biologic cargo was designed (FIG. 4A). In an effort to build a construct wherein the cargo production would be limited to cells expressing a desired miRNA, the 2A protein was incorporated downstream of the long terminal repeat of the lentivirus genome and included two miR-21 target sites into the 3′ untranslated region of the 2A transcript. In addition to this, a weak 5′ splice site was also incorporated into the 2A transcript (denoted by the hatched line in FIG. 4A). As the 3′ splice site was incorporated downstream of the miR-21 targeting elements, alternative spliced transcripts, unlike 2A, would not be subjected to RNAi-mediated silencing as the transcript would not include the miRNA silencing element (MSE). However, downstream of the 3′ splice site, the 2A-dependent PRS stem loop (SL) was incorporated to be encoded in frame with a Myc-epitope tagged mCherry open reading frame. Based on this design, in the absence of miR-21, 2A expression is anticipated to be high and subsequently induce a frameshift in the translation of mCherry, rendering the protein out of frame. In contrast, in the presence of miR-21, 2A expression is silenced and the ribosomal frame shift does not occur enabling canonical production of mCherry. As a result, mCherry expression can proceed in frame, but only in miR-21-expressing cells. To formally demonstrate this, lentiviruses encoding this miRNA-dependent circuit were generated and used to transduce either wild type MDCK cells or MDCK cells lacking miR-21 which have been previous described (MDCK^{miR-21-/-}) 14. These efforts yielded mCherry expression only in the presence of miR-21 (FIG. 4B).

### Example 3. RFE-Based miRNA-Dependent Expression Construct

Human Embryonic Kidney 293T cells (HEK293T) possess a unique combination of kidney epithelial cell and fibroblast characteristics. Recent miRNA profiling of HEK293T cells has demonstrated the presence of miR-21 and an absence of miR-124. Conversely, U-87 human glioblastoma cells, originating from a malignant brain tumor, exhibit the opposite miRNA expression pattern, with abundant miR-124 expression but an absence of miR-21. Using these two cell systems, the functionality of a miR-21- and a miR-124-specific RNA-based circuit was tested in the context of either Adeno-associated virus (AAV) serotype 2 (AAV2) or a lentivirus-based vector. For both constructs, the 2A gene product and the mCherry cargo were under the control of a CMV promoter. The 2A gene is encoded as an uninterrupted open reading frame (ORF) in which the 3′UTR contained either two miRNA target sites complementary to either miR-21 or miR-124. The mCherry transcript is encoded as an alternatively spliced construct, in which removal of the intron removes the miRNA silencing elements (MSEs) but retains the programmable ribosomal shift element in frame with mCherry. In the presence of 2A, mCherry would shift to a-1 frame resulting in a loss of the functional protein.

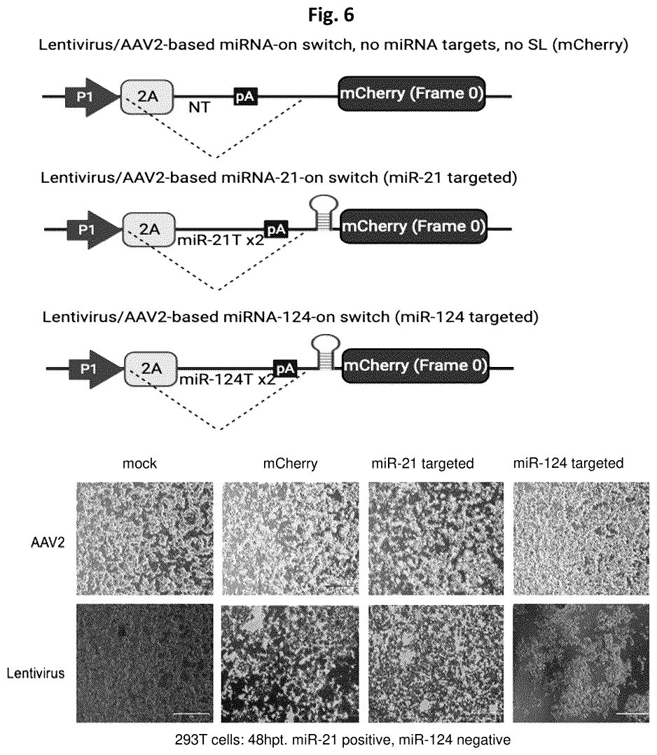

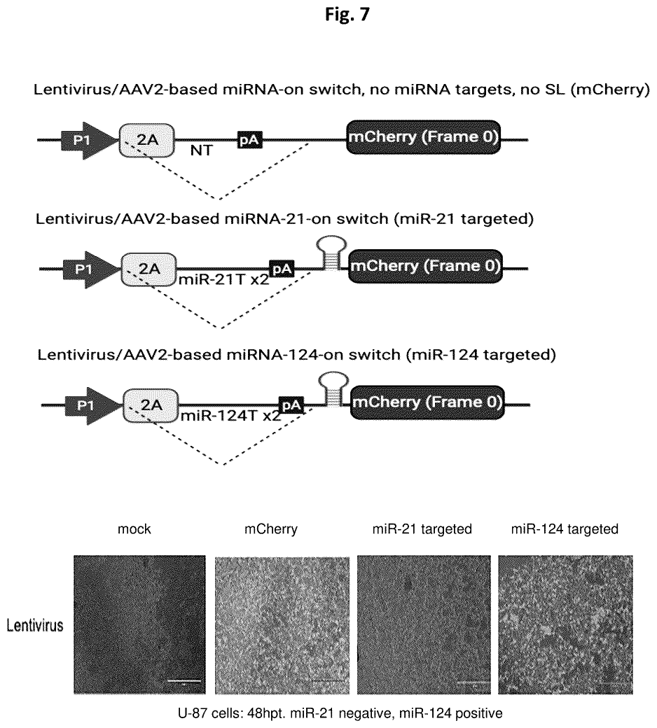

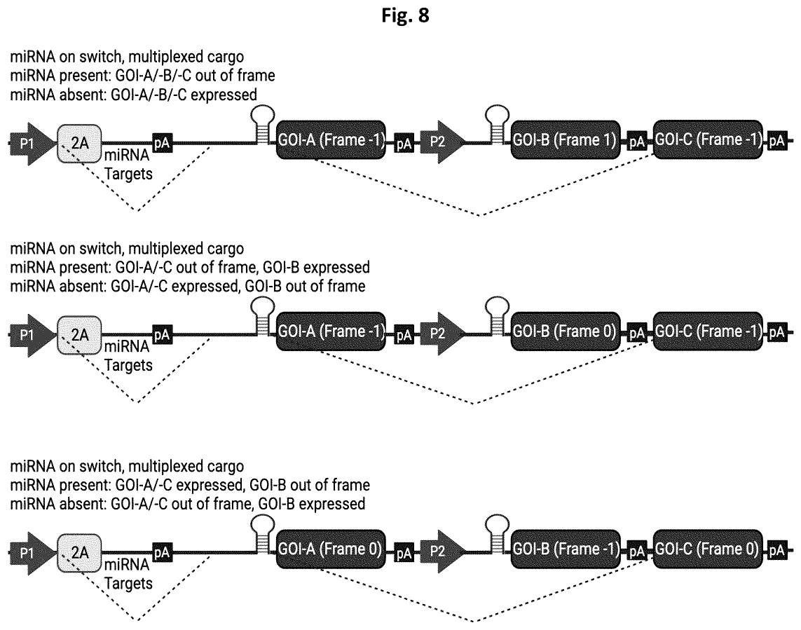

To test these constructs, 293T cells were treated with AAV2 (MOI 50) or lentiviruses (MOI 5) containing either no 2A protein (mCherry) or the circuits depicted containing either miR-21 target sites or miR-124 target sites. Five days post transduction, fluorescence was measured by microscopy to reveal that amongst the 2A-containing constructs, mCherry expression was confined to the construct containing miR-21 target sites for both the AAV2— and lentivirus-based constructs (FIG. 6). This experiment was then repeated in U-87 cells. However, as AAV2 was unable to transduce this glioblastoma cell line, testing was limited to the lentivirus-based miR-21 and miR-124 constructs. U-87 cells were transduced with the indicated lentiviruses (MOI 5) and five days later were analyzed by microscopy. These data demonstrate that in these cells, only the miR-124 based RNA lentivirus expresses mCherry (FIG. 7).

### Example 4. RFE-Based miRNA-Dependent Expression Construct Using a Bi-Directional Promoter

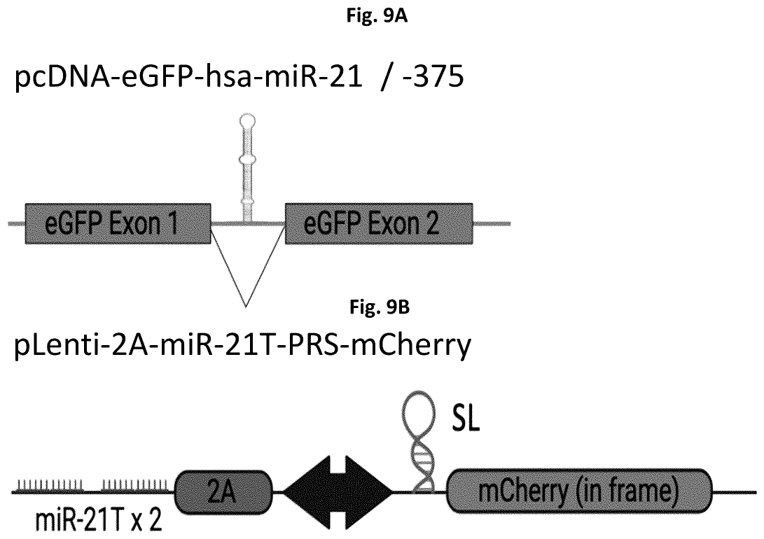

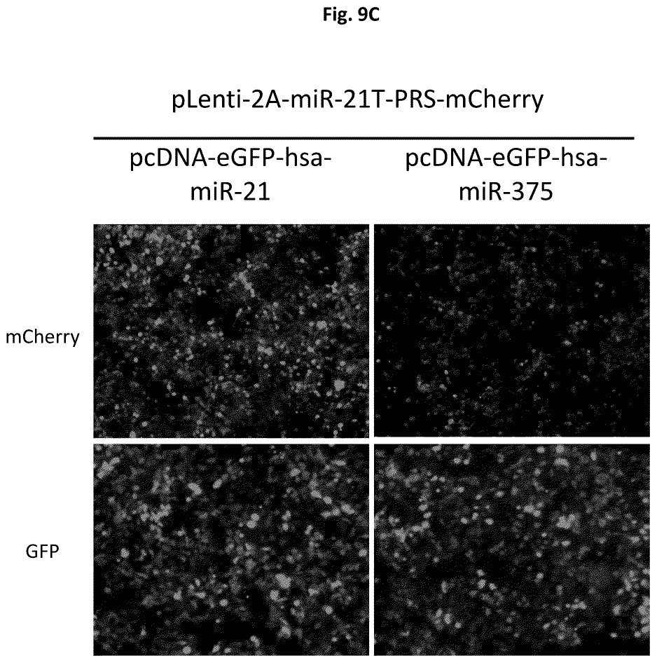

A plasmid encoding eGFP as two independent exons was constructed wherein the intron contained the pre-cursors for either Homo sapien miR-21 (hsa-miR-21) or miR-375 (hsa-miR-375) (FIG. 9A). A lentiviral genome comprising a bi-directional promoter was designed to express an EMCV 2A frameshift protein harboring two miR-21 target sites in the 3′ UTR and an in-frame mCherry transcript harboring a 5′ stem loop (SL) comprising the EMCV programable ribosomal shift (PRS) element (FIG. 9B). The human promoter for 3-phosphoglycerate kinase (hPGK) (SEQ ID NO. 13) was used in one direction and a minimal cytomegalovirus (CMV) promoter (SEQ ID NO. 14) was used in the other direction. To test these constructs, 293 cells were co-transfected with the pLenti-2A-miR-21-PRS-mCherry plasmid and a pcDNA-eGFP construct expressing either miR-21 or miR-375. Three days post treatment, fluorescent microscopy of the co-transfected 293 cells was performed. Images taken at 3 days post treatment are shown in FIG. 9C. The results showed efficient mCherry expression in the presence of miR-21.

### Example 5. RFE-Based miRNA-Dependent Expression Construct in a Sendai Virus

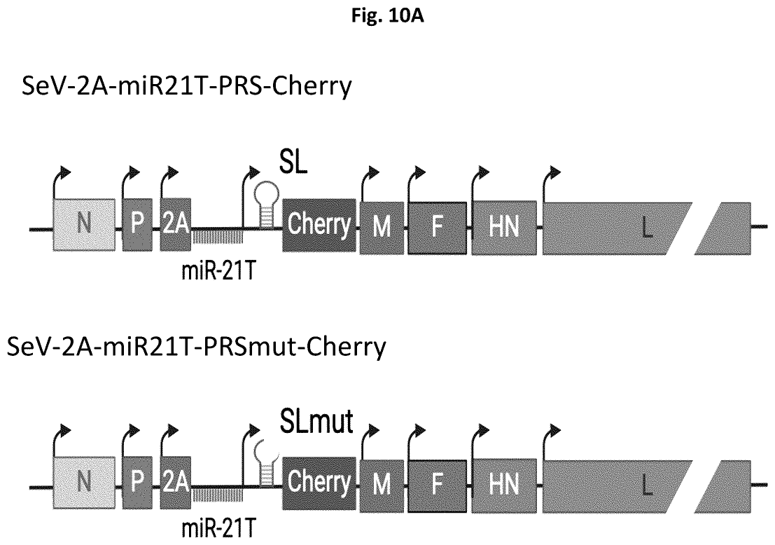

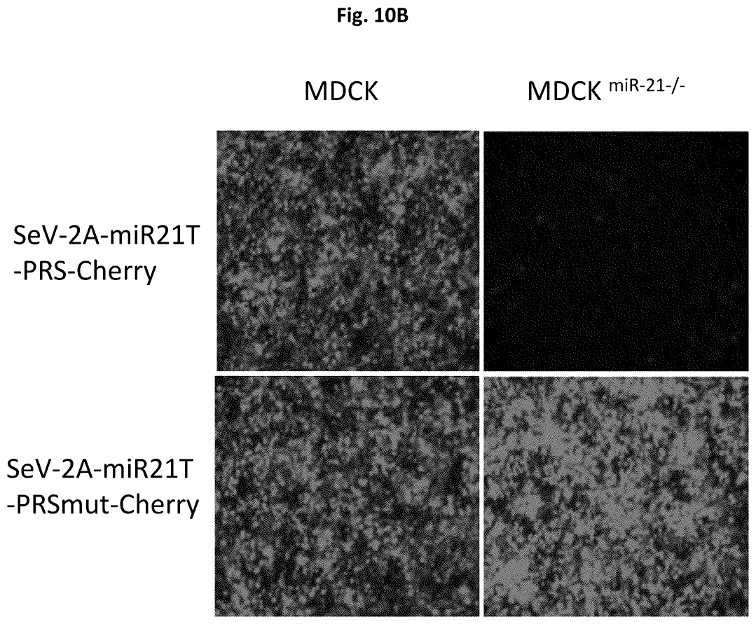

In this Example, Sendai virus was used as a delivery vector. Sendai virus is an advantageous delivery vector as it encodes its genes as individual cistrons and is therefore easy to engineer with the miRNA-dependent circuit. An engineered Sendai virus genome was constructed to encode a microRNA-21 (miR-21)-targeted (miR-21T) transcript encoding the EMCV 2A protein (2A) and an in-frame mCherry transcript harboring a 5′ stem loop (SL) comprising either a functional or mutated (SLmut) version of the EMCV programable ribosomal shift (PRS) element. 2A and mCherry were designed to express as individual transcripts introduced between the Nucleocapsid (N) and Phosphoprotein (P) viral genes, and the Matrix (M), Fusion (F), Hemagglutinin-Neuraminidase (HN) and Large (L) polymerase (FIG. 10A). mCherry expression was analyzed by fluorescent microscopy in wild type Madin-Darby Canine Kidney (MDCK) cells, or a clonal MDCK line in which miR-21 expression was disrupted (MDCK^{miR-21-/-}). Panels shown in FIG. 10B denote cells infected with the recombinant viruses denoted in FIG. 10A at a multiplicity of infection of 5 at 48 hours post infection. mCherry expression was not detected in the absence of miR-21 and a functional SL element was required for the off-switch.

### Example 6. In Vivo Evaluation of RFE-Based miRNA-Dependent Expression Construct in a Sendai Virus

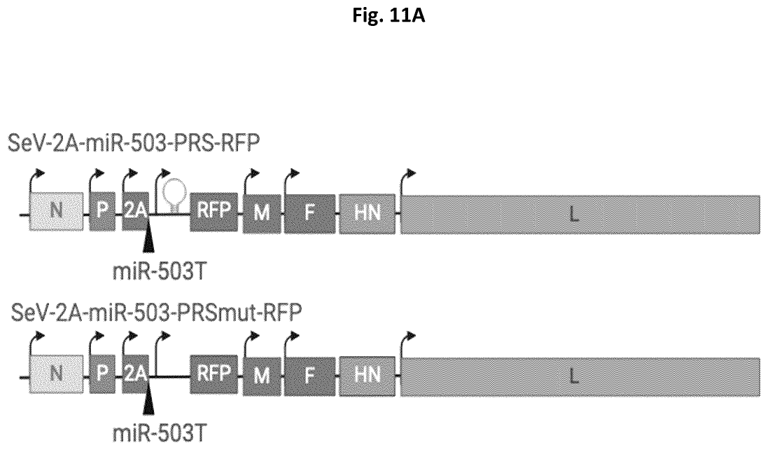

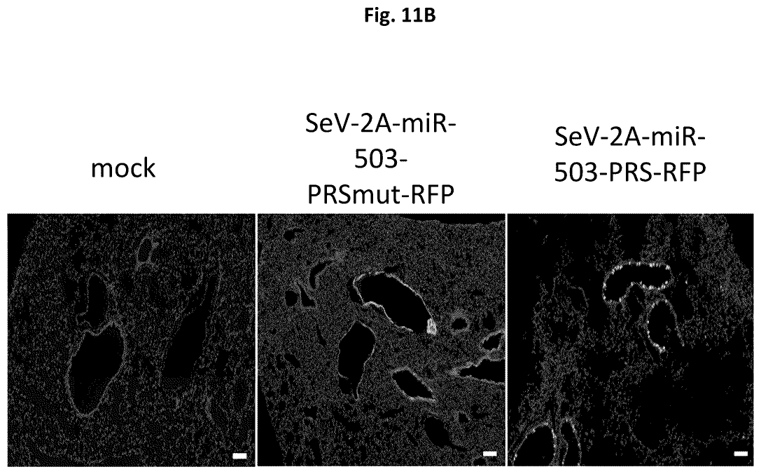

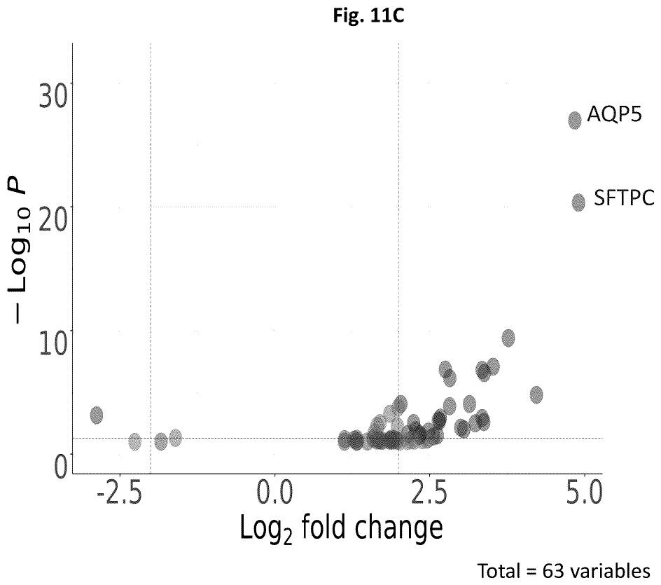

A Sendai virus vector was engineered to express the 2A protein with three miR-503 targets in its 3′ UTR (FIG. 11A). miR-503 expression is restricted to AT1/2 cells of the airways. In addition, the Sendai virus vector also encodes a red fluorescent protein (RFP) with either a wild type or mutated programable ribosomal shift (PRS) element in its 5′UTR. These two viral vectors were administered intranasally to Balb/c mice (400 hemagglutination units (HAU)) and lungs were harvested at 48 hours post treatment. Half of the lung was sectioned and analyzed for RFP expression by IHC (FIG. 11B). The other half of the lung was homogenized and sorted for RFP positive cells. RNA-Seq data of these two conditions were then aligned and were compared to identify genes enriched in the cells containing Sendai expressing the functional PRS element. FIG. 11C depicts a volcano plot of differentiated genes from RFP+ cells, showing that two genes, namely AQP5 and SFTPC, were significantly enriched. As both of these genes are predominantly expressed in AT1/2 cells, these data suggested that the vector functioned as designed.

### Example 7. Iron Response Element (IRE)-Based miRNA-Dependent Expression Construct

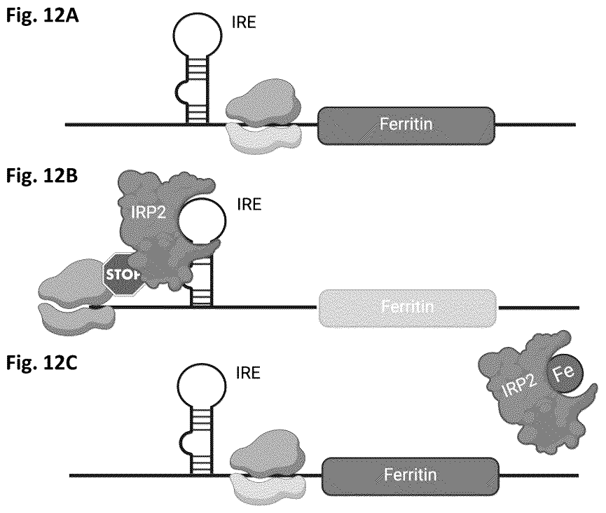

To generate an RNA-based circuit that functions only in the present of a desired cell, a biological control mechanism was exploited that evolved to respond to changes in intracellular iron levels. Mammalian cells express two proteins whose biology changes in response to iron concentrations, namely IRP1 (also known as IREB1) and IRP2 (also known as IREB2). In the present Example, IRP2 was leveraged to generate a miRNA-dependent circuit as it is smaller in size and lacks enzymatic activity in contrast to IRP1, although IRP1 could also be adapted for these same purposes. IRP2 undergoes proteome-mediated degradation in response to changing iron levels, which in turn impacts the translation rate of mRNA transcripts containing an RNA stem loop called the iron response element or IRE. In the presence of IRP2, binding to the IRE prevents mRNA translation, as characterized for the ferritin mRNA. The initial development of the ‘miRNA-only circuit’ leveraged the IRE found in the 5′ UTR of the ferritin mRNA, but other IREs may be leveraged. Using this system, wild type IRP2, or truncation mutants can be expressed to serve as a repressor for a target gene through the incorporation of an IRE. Moreover, while wild type IRP2 was used, IRP2 mutants that are non-responsive to iron levels but retain the capacity to bind IREs can also be used to ensure no disregulation in iron homeostasis. These mutants may comprise alanine substitutions for cysteines at position 120 (C120A), 375 (C375A), 578 (C578A), or 581 (C581A). Using this strategy, miRNA target sites can be incorporated into the 3′ UTR of the IRP2 transcript to create a circuit in which the target IRE-containing gene can only be translated when the cognate miRNA is present (FIGS. 12A-12C).

### Example 8. Repression of Payload Expression by IRP2

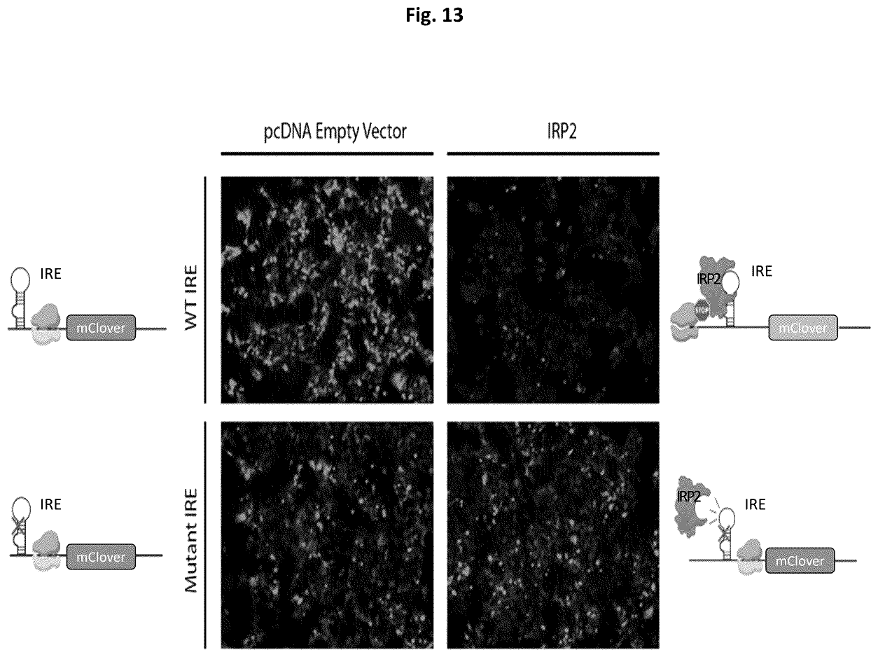

To demonstrate the functionality of this miRNA-dependent circuit, plasmids encoding either an IRE-containing mClover or IRP2 were generated. Plasmids encoding mClover contained either a wild-type IRE element in the 5′UTR (IRE-mClover) or a mutated IRE in the same position (IREmut-mClover). Plasmids encoding IRP2 were generated either with or without miRNA target sites. Initial characterization of these plasmids was performed by co-transfecting 293T cells with IRE-mClover or IREmut-mClover with either empty plasmid or one encoding IRP2 in the absence of miRNA target sites. These data demonstrated that IRP2 repressed mClover expression only when the wild-type IRE was present (FIG. 13).

### Example 9. Evaluation of IRE-Based miRNA-Dependent Expression Construct in 293T Cells

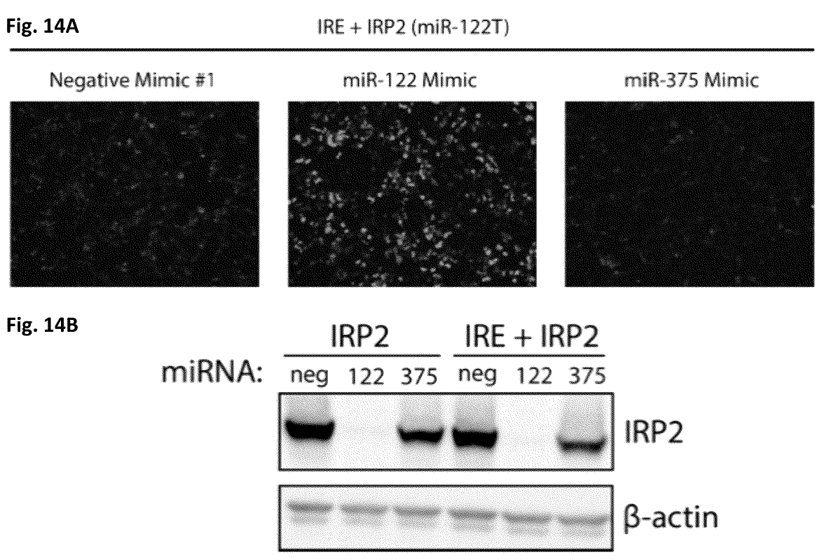

Following this initial proof-of-concept data, next a plasmid expressing IRP2 that encompassed two perfect target sites for microRNA-122 (miR-122) was generated, herein denoted pIRP2-miR-122T. Then, pIRP2-miR-122T and IRE-mClover were co-transfected into 293T cells alongside either a scrambled miRNA mimetic (Negative), a miR-122 mimic, or a miR-375 mimic. 48 hours post transfection, fluorescent microscopy demonstrated that robust green fluorescence emitted from mClover was confined to only conditions where miR-122 was present (FIG. 14A). These data could be further confirmed at the protein level, as western blots from these conditions showed a complete silencing of IRP2-miR-122T in the presence of the cognate miRNA (FIG. 14B). It is possible that increasing the number of miR-122 target sites may provide even greater control of this biological circuit.

As IRP2 is a host gene, the proposed circuit is unlikely to generate any unwanted engagement of the immune response and should be adaptable to a diverse number of vector platforms. Given the unmet need to mitigate off-target effects of such therapeutics, it is anticipated that this “miRNA-only” circuit has the potential to enable countless new treatments in the gene therapy space.

### Example 10. Design of IRE-Based miRNA-Dependent Expression Construct

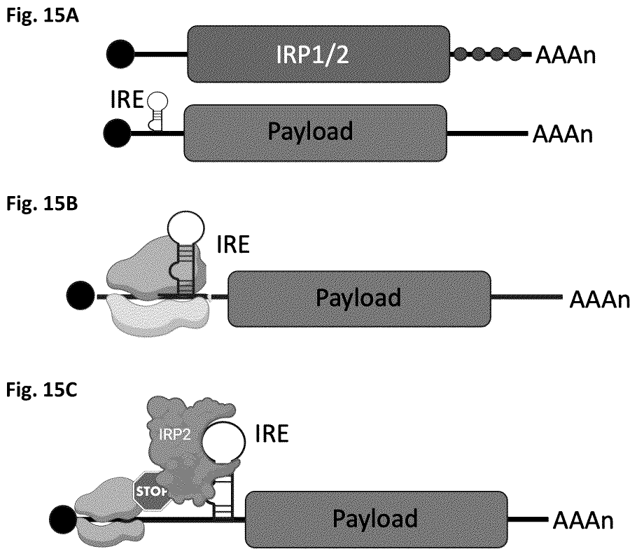

Herein is described a two-component system that enables the expression of a genetic payload only in the presence of a specific miRNA. This system consists of two key elements: (1) an IRE-binding protein (e.g., IRP1, IRP2) harboring a 3′ UTR containing two or more perfectly complementary target sites for the desired miRNA, and (2) a payload gene with a 5′ UTR harboring an iron-responsive element (IRE) (FIG. 15A). In this design, the presence or absence of the target miRNA dictates whether the payload gene is expressed. When the target miRNA(s) are present, it silences IRP2, thereby leaving the IRE unbound and enabling ribosomal translocation and subsequent payload expression (FIG. 15B). Coversely, when the miRNA(s) corresponding to the target sites in the 3′UTR of IRP2 are absent, IRP2 silencing does not occur, resulting in sufficient expression to engage the 5′ UTR IRE of the genetic payload, blocking ribosomal translacation and preventing expression (FIG. 15C).

### Example 11. Evaluation of IRE-Based miRNA-Dependent Expression Construct in 293T Cells

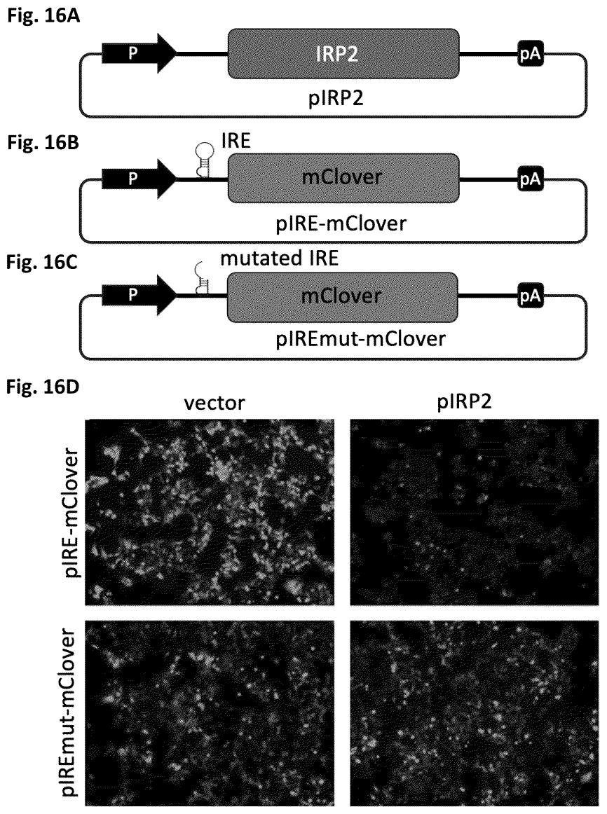

To assess the functionality of this putative miRNA-dependent circuit, two plasmids were synthesized representing the core components of the aforementioned elements one and two. For element one, the ORF encoding human IRP2 was synthesized into a eukaryotic expression vector, in the absence of any 5′ or 3′ UTR motifs (pIRP2) (FIG. 16A). For element two, mClover3 (herein referred to simply as mClover) was used as the ‘genetic payload’. The sequence of mClover3 derives from the Aequorea victoria green fluorescent protein (GFP), similar to other GFP-based variants, but was specifically designed for improved stability and brightness. To evaluate the specificity of IRP2/IRE-mediated translational repression, two mClover-expressing plasmids were generated: one containing the wild-type 5′ UTR of the ferritin gene with an intact IRE element (pIRE-mClover) and another with the same UTR but harboring a deletion in the IRE that disrupts IRP1/2-mediated repression (pIREmut-mClover) (FIGS. 16B-16C).

To assess IRP-mediated repression of IRE-containing transcripts, HEK293 cells were co-transfected with equimolar amounts of either pIRE-mClover or pIREmut-mClover, along with either an empty vector or pIRP2 (FIG. 16D). mClover expression was analyzed 48 hours post-transfection (48 hpt) using an EVOS fluorescent microscope. These data revealed robust fluorescence in cells co-transfected with pIRE-mClover and vector, which was abolished in the presence of pIRP2. In contrast, fluorescence remained detectable in cells transfected with pIREmut-mClover, regardless of whether vector or pIRP2 was present. Together, these findings confirm that the wild-type IRE element within the 5′ UTR of mClover is sufficient to mediate IRP2-dependent translational repression.

### Example 12. Evaluation of IRE-Based miRNA-Dependent Expression Construct in HEK293 Cells

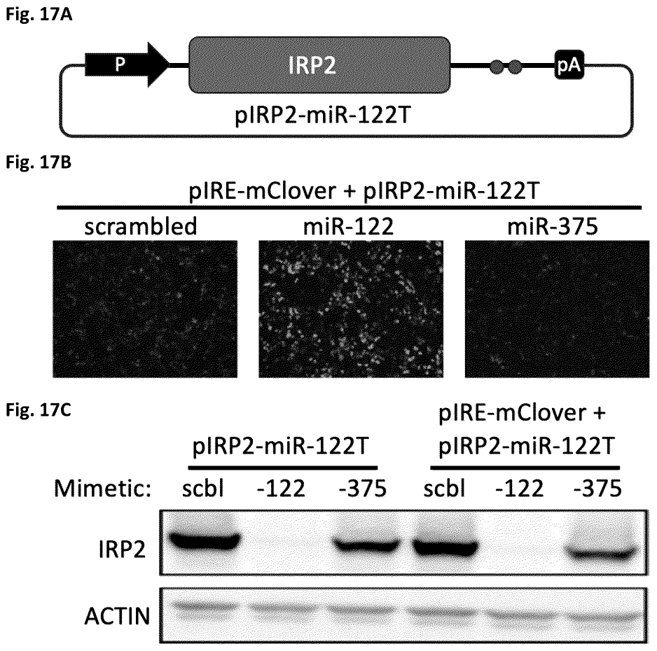

Building upon this initial proof-of-concept data, a modified pIRP2 construct was engineered to contain two perfect target sites for microRNA-122 (miR-122), a liver-specific miRNA, hereafter referred to as pIRP2-miR-122T (FIG. 17A). Given the transfection challenges associated with hepatocyte-derived cell lines, the dynamics of miRNA targeting were examined within the IRP/IRE circuit using Drosha-deficient HEK293 cells. These cells lack the ability to process precursor miRNAs, rendering them miRNA-deficient. Importantly, these miRNA-deficient cells retain all necessary components for miRNA-mediated regulation, which can be restored through the transfection of synthetic miRNA mimetics-chemically synthesized small RNA duplexes designed to imitate endogenous mature miRNAs.

Utilizing this in vitro platform, these miRNA-deficient cells were co-transfected with either a scrambled control (scrambled), miR-122 (miR-122), or miR-375 (miR-375), a pancreatic islet cell-specific miRNA, using miRNA mimetics according to the manufacturer's instructions. Three days following the introduction of the aforementioned small RNA mimetics, cells were additionally co-transfected with pIRP2-miR-122T and pIRE-Cover and assessed for fluorescent expression 48 hrs thereafter (FIG. 17B). These data showed minimal mClover expression when either the scrambled or miR-375 mimetics were introduced, whereas robust mClover expression was readily observed in conditions where miR-122 was present. While these results are consistent with the expected output of the IRE/IRP circuit design, the circuit biology was further validated by performing a western blot to assess IRP2 expression using whole-cell protein extracts from each condition, as well as conditions where only pIRP2-miR-122T were introduced (FIG. 17C). These data confirmed that IRP2 protein derived from the pIRP2-miR-122T plasmid was readily detectable in all conditions lacking miR-122, but were not detectable when miR-122 was present (FIG. 17C).

### Example 13. IRE-Based miRNA-Dependent Expression Construct in a Lentivirus

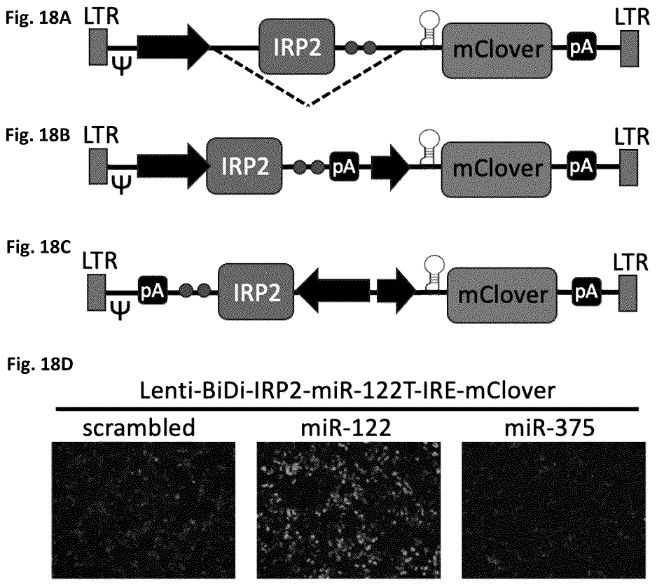

Building on the performance of the miR-122-dependent circuit, its function was validated in a system independent of plasmid-based transfection. To achieve this, it was incorporated into a lentiviral-based delivery system, though other vector platforms (e.g., a Sendai virus) could also be utilized. Additionally, the design of element one and element two within the vector offers considerable flexibility, allowing for multiple configurations to optimize expression. For example, a single promoter can drive the expression of both elements through alternative splicing, generating distinct transcripts from a shared regulatory sequence (FIG. 18A). Alternatively, each element can be placed under the control of separate, dedicated promoters arranged either in tandem (FIG. 18B) or in a bi-directional configuration to ensure no ‘read-through’ products are generated (FIG. 18C). This versatility makes the system highly adaptable to different experimental and therapeutic contexts, including use in AdV, AAV, or other vector platforms.

To validate the IRE/IRP-based circuit in the context of vector transduction, a bi-directional design was employed for each element (FIG. 18C). To generate this vector using the IRP2-miR-122T, a plasmid containing long terminal repeat (LTR) sequences flanking the circuit elements was constructed. The plasmid also comprised a bi-directional (BiDi) promoter centrally positioned between the LTRs, with a CMV promoter and enhancer driving IRP2-miR-122T expression and a CMV minimal promoter controlling IRE-mClover transcription. Additional vector components included polyadenylation (polyA) sites located 3′ to each transcript and the psi (Y) packaging sequence near the 5′ LTR for efficient viral RNA encapsidation. This plasmid is referred to as Lenti-BiDi-IRP2-miR-122T-IRE-mClover.

Lentiviral vector production was carried out in HEK293T cells, which were co-transfected with the Lenti-BiDi-IRP2-miR-122T-IRE-mClover plasmid, along with packaging plasmids encoding Gag, Pol, Env, and VSV-G, to pseudotype the virus for broad tropism. Following transfection, viral particles were collected from the supernatant, quantified by qPCR and p24 ELISA, and subsequently used for transduction. To assess circuit function, again miRNA-deficient cells pretreated with either scrambled, miR-122, or miR-375 mimetics prior to transduction with Lenti-BiDi-IRP2-miR-122T-IRE-mClover were used. Fluorescence analysis 48 hours post-transduction revealed that mClover expression occurred exclusively in conditions where miR-122 was present, confirming the specificity of the circuit (FIG. 18D). These findings further validate the functionality of the IRE/IRP-based circuit in a viral vector system, demonstrating its potential for direct application in gene therapy to regulate genetic payloads with tissue-specific miRNA signatures.

### Example 14. IRE-Based miRNA-Dependent Expression Construct Using a Bi-Directional Promotor

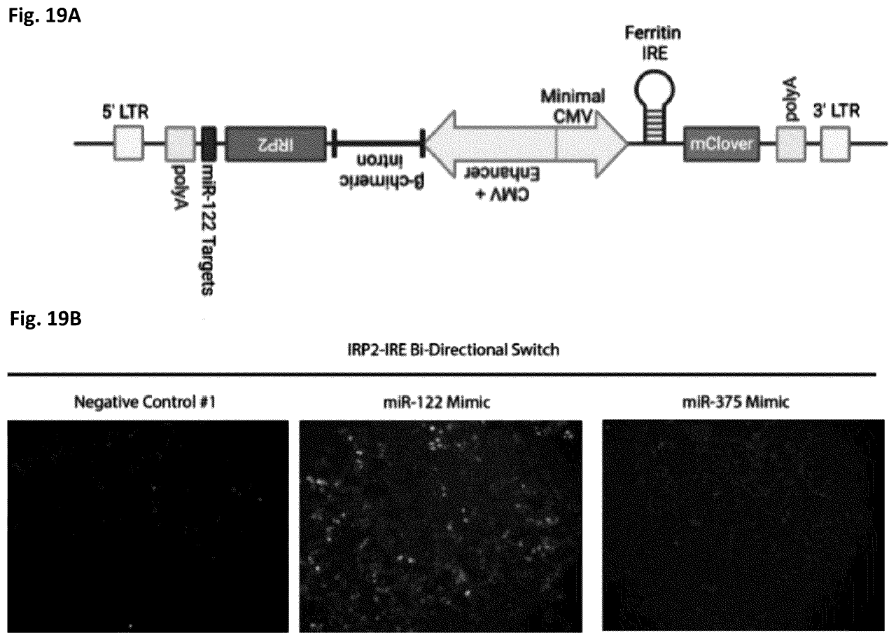

MiR-122-responsive lentivirus is enabled by an IRP2-IRE-bi-directional switch. A lentiviral genome features a central bi-directional promoter. Top strand transcription is driven by the minimal CMV immediate-early (IE) promoter, which lacks the endogenous enhancer motif, and controls expression of a fluorescent mClover gene. This gene includes the iron response element (IRE) from the human ferritin transcript within its 5′ UTR. Bottom strand transcription is regulated by the full CMV promoter (CMV+enhancer), driving expression of an intron-containing IRP2 transcript, which contains two fully complementary sites for miR-122. Both mClover and IRP2 have independent polyadenylation (polyA) signals, and the entire genetic circuit is flanked by lentiviral long terminal repeats (LTRs) (FIG. 19A). Fluorescent microscopy images of HEK293T cells treated with a scrambled miRNA mimetic (negative control #1), miR-122, or miR-375 and subsequently transduced with the lentivirus were obtained (FIG. 19B).

### Example 15. Evaluation of IRP2 Mutants with C-Terminal Truncations

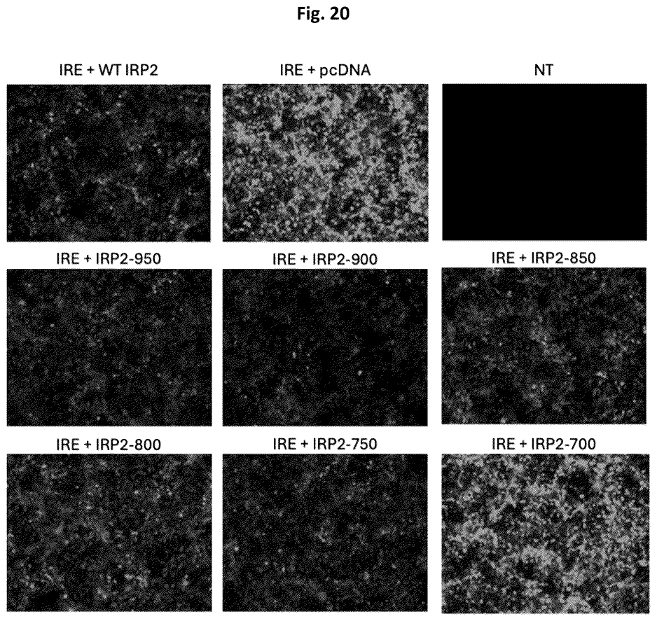

293T cells were co-transfected with a plasmid encoding enhanced green fluorescent protein (eGFP) containing a 5′ IRE alongside a second plasmid encoding wild type IRP2 (WT IRP2), or IRP2 C-terminal truncations. The C-terminal truncation mutants are denoted by the number of amino acids remaining from the full-length 963-amino-acid protein, and included IRP2-950 (IRP2 lacking the last 13 amino acids in the C-terminal end), IRP2-900 (IRP2 lacking the last 63 amino acids in the C-terminal end), IRP2-850 (IRP2 lacking the last 113 amino acids in the C-terminal end), IRP2-800 (IRP2 lacking the last 163 amino acids in the C-terminal end), IRP2-750 (IRP2 lacking the last 213 amino acids in the C-terminal end), and IRP2-700 (IRP2 lacking the last 263 amino acids in the C-terminal end). Negative controls included non-transfected (NT) or IRE-eGFP (IRE) co-transfected with an empty vector (pcDNA). eGFP expression in 293T cells was assessed using fluorescent microscopy 48 hours after the co-transfection. The results are presented in FIG. 20. The data suggest that the last 213 amino acids in the C-terminal end of IRP2 are not required for IRP2-mediated IRE repression.

### Example 16. Evaluation of IRP2 Mutants with Internal Deletions

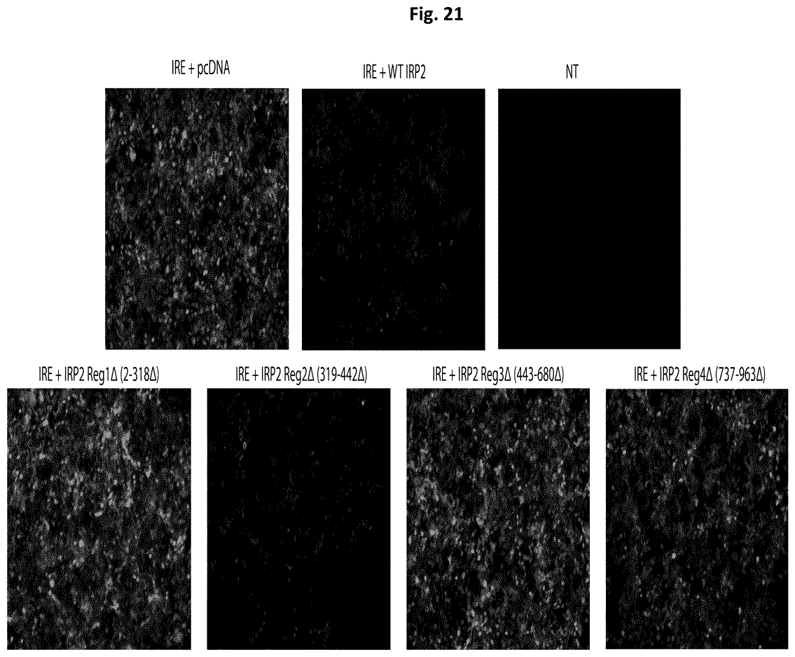

293T cells were co-transfected with a plasmid encoding eGFP containing a 5′ IRE alongside a second plasmid encoding WT IRP2, or IRP2 mutants harboring internal deletions. The tested IRP2 deletion mutants included IRP2 Reg1Δ (2-318Δ) (IRP2 lacking amino acids 2-318), IRP2 Reg2Δ (319-442Δ) (IRP2 lacking amino acids 319-442), IRP2 Reg3Δ (443-680Δ) (IRP2 lacking amino acids 443-680), and IRP2 Reg4Δ (737-963Δ) (IRP2 lacking amino acids 737-963). Negative controls included non-transfected (NT) or IRE-eGFP co-transfected with an empty vector (pcDNA). eGFP expression in 293T cells was assessed using fluorescent microscopy 48 hours after the co-transfection. The results are presented in FIG. 21. The data suggest that the residues 319-442 in IRP2 are not required for IRP2-mediated IRE repression.

### Example 17. Evaluation of IRP2 Mutants with Point Mutations

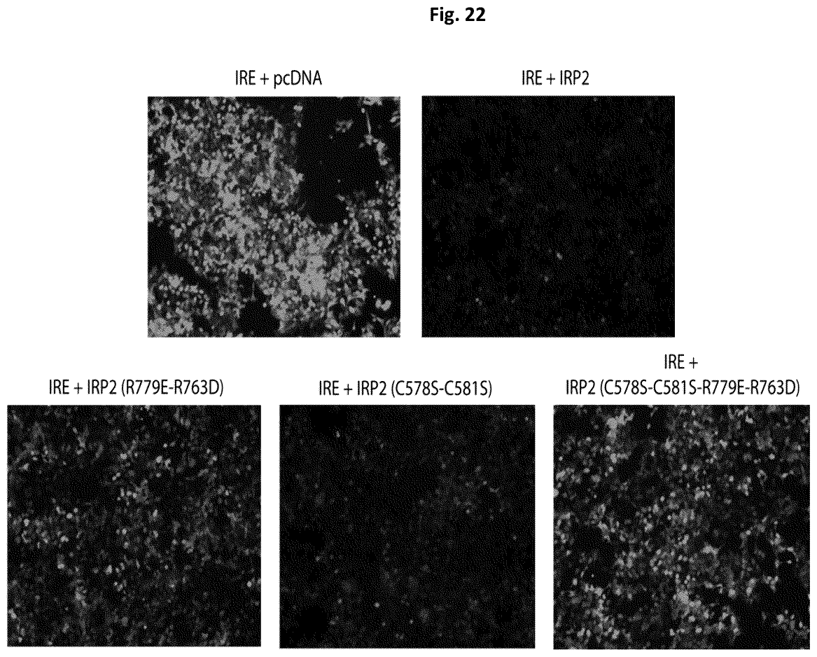

293T cells were co-transfected with a plasmid encoding eGFP containing a 5′ IRE alongside a second plasmid encoding WT IRP2, an empty vector (pcDNA), or one of three IRP2 mutants designed to disrupt FBXL5-mediated degradation of IRP2, which is the mechanism responsible for IRP2 degradation in high iron conditions (e.g. IRP2 iron sensitivity). These mutants are R779E-R763D mutations, C578S-C581S mutations, and a combined C578S-C581S-R779E-R763D mutant. eGFP expression in 293T cells was assessed using fluorescent microscopy 48 hours after the co-transfection. The results are presented in FIG. 22. The data suggest that R779 and R763 are essential for IRP2-mediated IRE repression, while C578 and C581 are not essential. IRP2-C578S-C581S can be a functional IRP2 variant that is not responsive to endogenous iron levels.

### Example 18. Evaluation of IRE-Based miRNA-Dependent Expression Construct in Dicer-Deficient 293T Cells

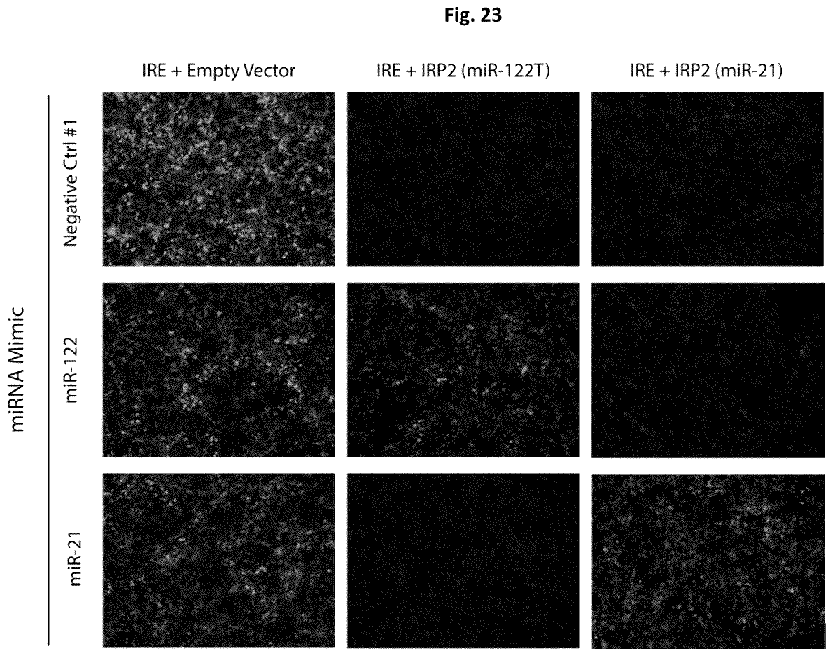

Dicer-deficient 293T cells were co-transfected with a plasmid encoding eGFP containing a 5′ IRE alongside a second plasmid comprising an empty vector (pcDNA), or encoding WT IRP2 with three miRNA target sites for either miR-122 or miR-21. 24 hours prior to treatment, cells were transfected with a scrambled miRNA (Negative ctrl #1), or mimetics for miR-122 or miR-21. eGFP expression in 293T cells was assessed using fluorescent microscopy 48 hours after the co-transfection. The results are presented in FIG. 23. The data suggest that miRNA silencing of IRP2 is selective based on the miRNA target site used and miRNA available in the cell, and the combined system can be used to turn on gene expression in the presence of particular miRNA.

### Example 19. Lin28-Based miRNA-Dependent Expression Construct

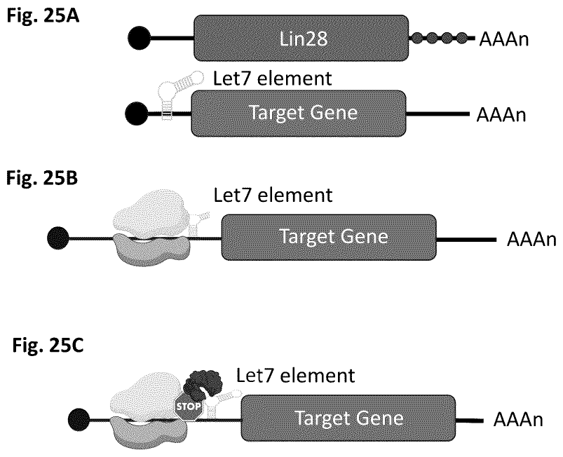

A schematic representation of the design of a Lin28-based miRNA-dependent genetic circuit is shown in FIGS. 25A-25C. The system consists of (1) a gene encoding an RNA-binding protein, Lin28, containing a 3′ UTR with two or more perfectly complementary target sites for the desired miRNA, and (2) a payload gene with a 5′ UTR harboring an RNA element derived from a let-7 miRNA precursor (pre-let-7). In the presence of the target miRNA, Lin28 is silenced via miRNA-mediated degradation or translational repression. As a result, the pre-let-7 element in the payload's 5′ UTR remains unbound, allowing ribosomal translocation and subsequent payload expression. In the absence of the target miRNA, Lin28 remains expressed and actively binds to the pre-let-7 element in the 5′ UTR of the payload gene. This binding blocks ribosomal translocation, preventing translation and effectively silencing the payload gene.

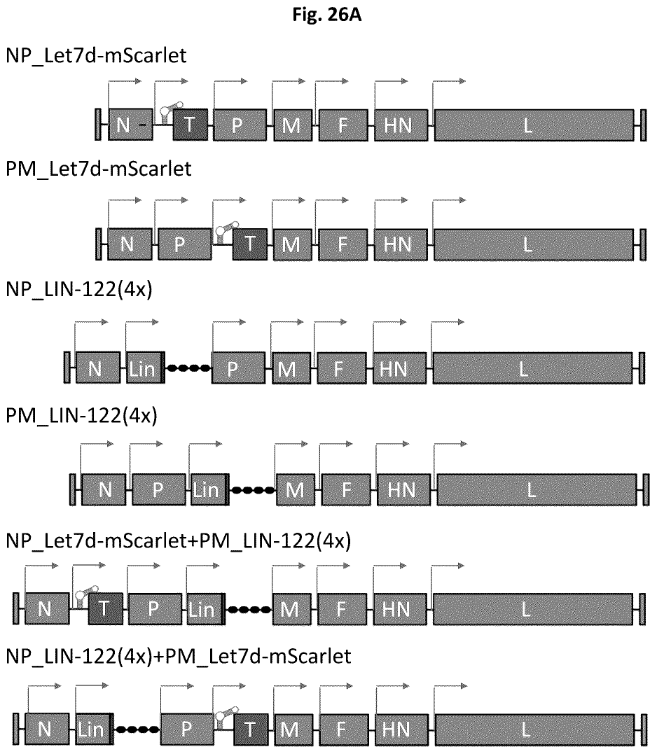

The Lin28-based miRNA-dependent genetic circuit was tested in Sendai vectors to express a reporter protein, mScarlet. Six Sendai vectors carrying the LET7D mScarlet cassette and the Lin28A cassette in different arrangements were designed (FIG. 26A). The Lin28A cassette includes four miR-122 target sites in its 3′ UTR. The construct NP_plet7d-mScarlet encodes LET7D mScarlet between N and P. The construct PM_plet7d-mScarlet carries the same cassette between P and M. The construct NP_LIN-122T (4×) encodes Lin28A with four tandem miR-122 sites at the N to P junction, and PM_LIN-122T (4×) encodes the same cassette between P and M. Two combined vectors were also generated. In NP_plet7d-mScarlet PM_LIN-122T (4×), LET7D mScarlet is placed between N and P while Lin28A-122T is placed between P and M. In the reciprocal arrangement, NP_LIN-122T (4×) PM_plet7d-mScarlet, the Lin28A-122T cassette is positioned between N and P and the LET7D mScarlet cassette is positioned between P and M.

A549 cells lacking miR-122 and Huh7.5 cells expressing miR-122 were infected with the six described vectors at a multiplicity of infection of three. mScarlet expression was assessed using fluorescent microscopy 24 hours after infection. The results are shown in FIG. 26B. The LET7D mScarlet only vectors NP_plet7d-mScarlet and PM_plet7d-mScarlet produced red fluorescence in both cell types. The Lin28A only vectors NP_LIN-122T (4×) and PM_LIN-122T (4×) produced no signal, consistent with the absence of an mScarlet gene. The combined construct NP_plet7d-mScarlet PM_LIN-122T (4×) produced mScarlet fluorescence selectively in Huh7.5 cells, indicating miR-122 dependent activation of the LET7D module. The reciprocal construct NP_LIN-122T (4×) PM_plet7d-mScarlet showed no detectable fluorescence in either cell type, demonstrating that gene order within the Sendai genome strongly influences output from the coupled system.

### Example 20. MS2-Based miRNA-Dependent Expression Construct

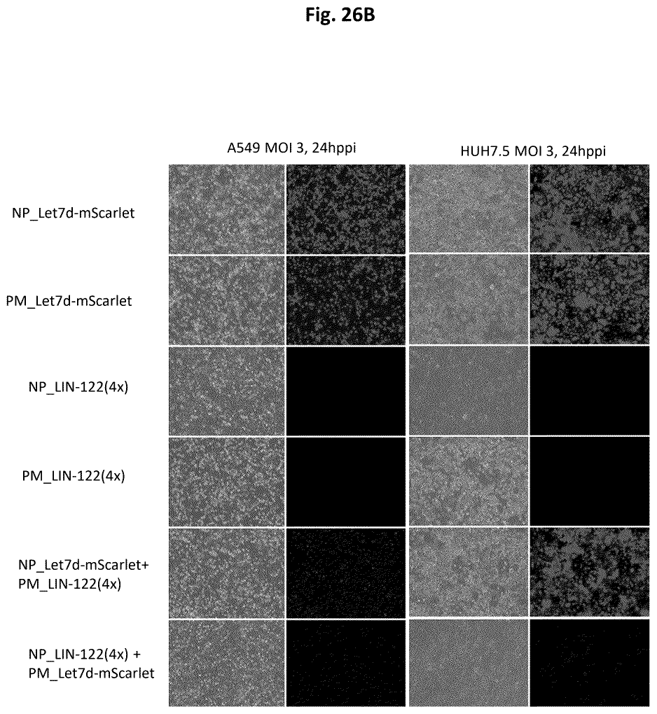

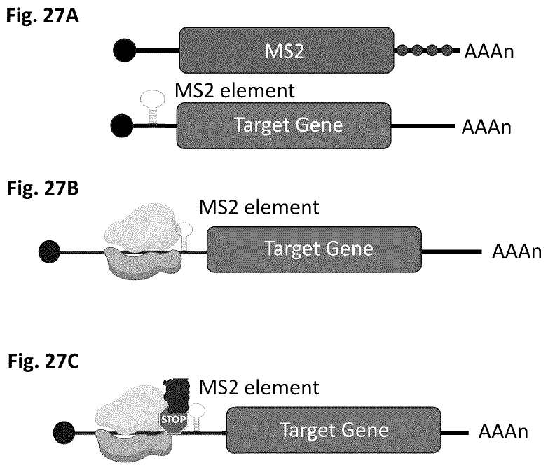

A schematic representation of the design of a MS2-based miRNA-dependent genetic circuit is shown in FIGS. 27A-27C. The system consists of (1) a gene encoding an RNA-binding protein, MS2 bacteriophage coat protein (MS2), containing a 3′ UTR with two or more perfectly complementary target sites for the desired miRNA, and (2) a payload gene with a 5′ UTR harboring an MS2 responsive RNA element. In the presence of the target miRNA, MS2 is silenced via miRNA-mediated degradation or translational repression. As a result, the MS2 responsive RNA element in the payload's 5′ UTR remains unbound, allowing ribosomal translocation and subsequent payload expression. In the absence of the target miRNA, MS2 remains expressed and actively binds to the MS2 responsive RNA element in the 5′ UTR of the payload gene. This binding blocks ribosomal translocation, preventing translation and effectively silencing the payload gene.

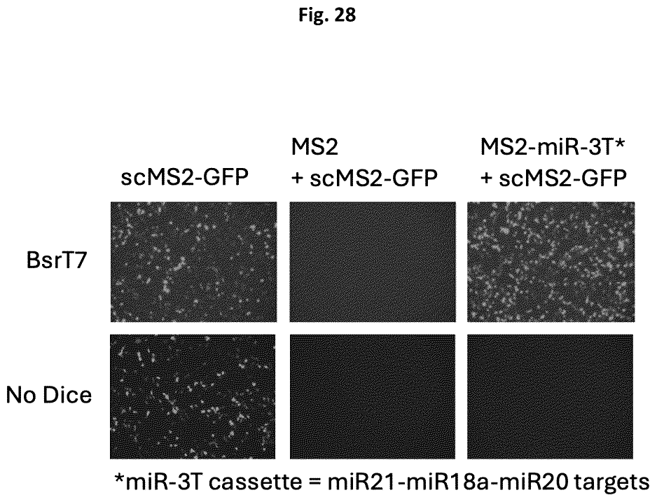

miRNA positive BsrT7 cells and miRNA deficient NoDice 293T cells were transfected with a GFP reporter containing a 5′ MS2 responsive RNA element (scMS2 GFP). Cells were transfected with scMS2 GFP alone, scMS2 GFP plus an MS2 expression plasmid, or scMS2 GFP plus an MS2 plasmid containing target sites for miR-21, miR-18a, and miR-20 in its 3′ untranslated region. GFP expression was assessed using fluorescent microscopy 48 hours after transfection. The results are shown in FIG. 28. Reporter expression is suppressed when MS2 is present and is restored only when MS2 is selectively silenced by the endogenous miRNAs.

### Example 21. PP7-based miRNA-dependent Expression Construct

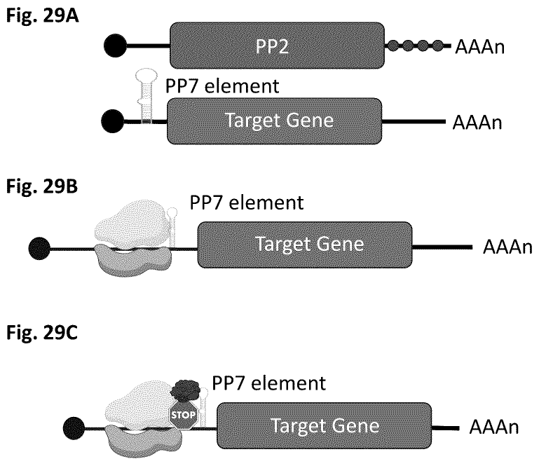

A schematic representation of the design of a PP7-based miRNA-dependent genetic circuit is shown in FIGS. 29A-29C. The system consists of (1) a gene encoding an RNA-binding protein, PP7 bacteriophage coat protein (PP7), containing a 3′ UTR with two or more perfectly complementary target sites for the desired miRNA, and (2) a payload gene with a 5′ UTR harboring an PP7 responsive RNA element. In the presence of the target miRNA, PP7 is silenced via miRNA-mediated degradation or translational repression. As a result, the PP7 responsive RNA element in the payload's 5′ UTR remains unbound, allowing ribosomal translocation and subsequent payload expression. In the absence of the target miRNA, PP7 remains expressed and actively binds to the PP7 responsive RNA element in the 5′ UTR of the payload gene. This binding blocks ribosomal translocation, preventing translation and effectively silencing the payload gene.

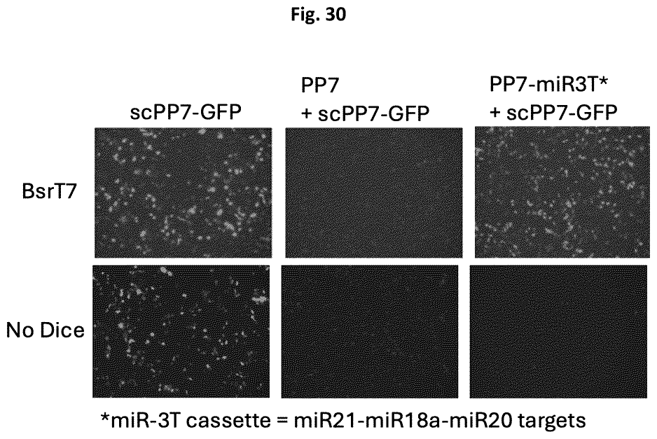

miRNA positive BsrT7 cells and miRNA deficient NoDice 293T cells were transfected with a GFP reporter containing a 5′ PP7 responsive RNA element (scPP7 GFP). Cells were transfected with scPP7 GFP alone, scPP7 GFP plus a PP7 expression plasmid, or scPP7 GFP plus a PP7 plasmid containing target sites for miR-21, miR-18a, and miR-20 in its 3′ untranslated region. GFP expression was assessed using fluorescent microscopy 48 hours after transfection. The results are shown in FIG. 30. Reporter expression is suppressed when PP7 is present and is restored only when PP7 is selectively silenced by the endogenous miRNAs.

### Example 22. U1A-Based miRNA-Dependent Expression Construct

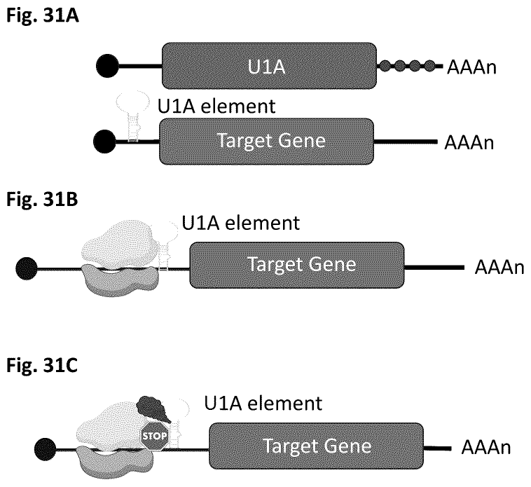

A schematic representation of the design of a U1A-based miRNA-dependent genetic circuit is shown in FIGS. 31A-31C. The system consists of (1) a gene encoding an RNA-binding protein, U1 small nuclear ribonucleoprotein A (U1A), containing a 3′ UTR with two or more perfectly complementary target sites for the desired miRNA, and (2) a payload gene with a 5′ UTR harboring an U1A responsive RNA element. In the presence of the target miRNA, U1A is silenced via miRNA-mediated degradation or translational repression. As a result, the ULA responsive RNA element in the payload's 5′ UTR remains unbound, allowing ribosomal translocation and subsequent payload expression. In the absence of the target miRNA, U1A remains expressed and actively binds to the U1A responsive RNA element in the 5′ UTR of the payload gene. This binding blocks ribosomal translocation, preventing translation and effectively silencing the payload gene.

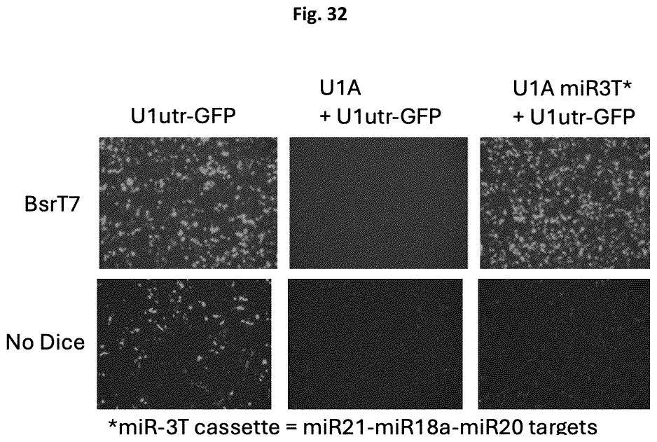

miRNA positive BsrT7 cells and miRNA deficient NoDice 293T cells were transfected with a GFP reporter containing a 5′ U1A responsive RNA element (U1utr GFP). Cells were transfected with U1utr GFP alone, U1utr GFP plus a U1A expression plasmid, or U1utr GFP plus a U1A plasmid containing target sites for miR-21, miR-18a, and miR-20 in its 3′ untranslated region. GFP expression was assessed using fluorescent microscopy 48 hours after transfection. The results are shown in FIG. 32. Reporter expression is suppressed when U1A is present and is restored only when U1A is selectively silenced by the endogenous miRNAs.

### REFERENCES

1 Paunovska, K., Loughrey, D. & Dahlman, J. E. Drug delivery systems for RNA therapeutics. Nat Rev Genet 23, 265-280 (2022).
2 Pardi, N., Hogan, M. J., Porter, F. W. & Weissman, D. mRNA vaccines-a new era in vaccinology. Nat Rev Drug Discov 17, 261-279 (2018).
3 Bulcha, J. T., Wang, Y., Ma, H., Tai, P. W. L. & Gao, G. Viral vector platforms within the gene therapy landscape. Signal Transduct Target Ther 6, 53 (2021).
4 Sung, Y. K. & Kim, S. W. Recent advances in the development of gene delivery systems. Biomater Res 23, 8 (2019).
5 Perez, J. T. et al. MicroRNA-mediated species-specific attenuation of influenza A virus. Nat Biotechnol 27, 572-576 (2009).
6 Langlois, R. A. et al. MicroRNA-based strategy to mitigate the risk of gain-of-function influenza studies. Nat Biotechnol 31, 844-847 (2013).
7 Langlois, R. A., Varble, A., Chua, M. A., Garcia-Sastre, A. & tenOever, B. R. Hematopoietic-specific targeting of influenza A virus reveals replication requirements for induction of antiviral immune responses. Proc Natl Acad Sci USA 109, 12117-12122 (2012).
8 Benitez, A. A., Spanko, L. A., Bouhaddou, M., Sachs, D. & tenOever, B. R. Engineered Mammalian RNAi Can Elicit Antiviral Protection that Negates the Requirement for the Interferon Response. Cell Rep 13, 1456-1466 (2015).
9 Aguado, L. C. et al. Homologous recombination is an intrinsic defense against antiviral RNA interference. Proc Natl Acad Sci USA 115, E9211-E9219 (2018).
10 Bartel, D. P. Metazoan MicroRNAs. Cell 173, 20-51 (2018).
11 Bartel, D. P. MicroRNAs: genomics, biogenesis, mechanism, and function. Cell 116, 281-297 (2004).
12 tenOever, B. R. RNA viruses and the host microRNA machinery. Nat Rev Microbiol 11, 169-180 (2013).
13 Hill, C. H. et al. Structural and molecular basis for Cardiovirus 2A protein as a viral gene expression switch. Nat Commun 12, 7166 (2021).
14 Waring, B. M. et al. MicroRNA-Based Attenuation of Influenza Virus across Susceptible Hosts. J Virol 92 (2018).
15. Paunovska, K., Loughrey, D. & Dahlman, J. E. Drug delivery systems for RNA therapeutics. Nat Rev Genet 23, 265-280 (2022). doi: 10.1038/s41576-021-00439-4
16. Pardi, N., Hogan, M. J., Porter, F. W. & Weissman, D. mRNA vaccines-a new era in vaccinology. Nat Rev Drug Discov 17, 261-279 (2018). doi: 10.1038/nrd.2017.243
17. Bulcha, J. T., Wang, Y., Ma, H., Tai, P. W. L. & Gao, G. Viral vector platforms within the gene therapy landscape. Signal Transduct Target Ther 6, 53 (2021). doi: 10.1038/s41392-021-00487-6
18. Sung, Y. K. & Kim, S. W. Recent advances in the development of gene delivery systems. Biomater Res 23, 8 (2019). doi: 10.1186/s40824-019-0156-z
19. Perez, J. T. et al. MicroRNA-mediated species-specific attenuation of influenza A virus. Nat Biotechnol 27, 572-576 (2009). doi: 10.1038/nbt.1542
20. Langlois, R. A. et al. MicroRNA-based strategy to mitigate the risk of gain-of-function influenza studies. Nat Biotechnol 31, 844-847 (2013). doi: 10.1038/nbt.2666
21. Langlois, R. A., Varble, A., Chua, M. A., Garcia-Sastre, A. & tenOever, B. R. Hematopoietic-specific targeting of influenza A virus reveals replication requirements for induction of antiviral immune responses. Proc Natl Acad Sci USA 109, 12117-12122 (2012). doi: 10.1073/pnas. 1206039109
22. Benitez, A. A., Spanko, L. A., Bouhaddou, M., Sachs, D. & tenOever, B. R. Engineered Mammalian RNAi Can Elicit Antiviral Protection that Negates the Requirement for the Interferon Response. Cell Rep 13, 1456-1466 (2015). doi: 10.1016/j.celrep.2015.10.020
23. Aguado, L. C. et al. Homologous recombination is an intrinsic defense against antiviral RNA interference. Proc Natl Acad Sci USA 115, E9211-E9219 (2018). doi: 10.1073/pnas.1810229115
24. Bartel, D. P. Metazoan MicroRNAs. Cell 173, 20-51 (2018). doi: 10.1016/j.cell.2018.03.006
25. Bartel, D. P. MicroRNAs: genomics, biogenesis, mechanism, and function. Cell 116, 281-297 (2004). doi: 10.1016/s0092-8674 (04) 00045-5
26. tenOever, B. R. RNA viruses and the host microRNA machinery. Nat Rev Microbiol 11, 169-180 (2013). doi: 10.1038/nrmicro2971
27. Wallander, M. L., Leibold, E. A. & Eisenstein, R. S. Molecular control of vertebrate iron homeostasis by iron regulatory proteins. Biochim Biophys Acta 1763, 668-689 (2006). doi: 10.1016/j.bbamcr.2006.05.004
28. Leipuviene, R. & Theil, E. C. The family of iron responsive RNA structures regulated by changes in cellular iron and oxygen. Cell Mol Life Sci 64, 2945-2955 (2007). doi: 10.1007/s00018-007-7198-4
29. Muckenthaler, M. U., Galy, B. & Hentze, M. W. Systemic iron homeostasis and the iron-responsive element/iron-regulatory protein (IRE/IRP) regulatory network. Annu Rev Nutr 28, 197-213 (2008). doi: 10.1146/annurev.nutr.28.061807.155521
30. Volz, K. The functional duality of iron regulatory protein 1. Curr Opin Struct Biol 18, 106-111 (2008). doi: 10.1016/j.sbi.2007.12.010
31. Sanchez, M. et al. Iron regulation and the cell cycle: identification of an iron-responsive element in the 3′-untranslated region of human cell division cycle 14A mRNA by a refined microarray-based screening strategy. J Biol Chem 281, 22865-22874 (2006). doi: 10.1074/jbc.M603876200
32. Lam-Yuk-Tseung, S. & Gros, P. Distinct targeting and recycling properties of two isoforms of the iron transporter DMT1 (NRAMP2, Slc11A2). Biochemistry 45, 2294-2301 (2006). doi: 10.1021/bi052307m
33. Chen, S. C. & Olsthoorn, R. C. L. Relevance of the iron-responsive element (IRE) pseudotriloop structure for IRP1/2 binding and validation of IRE-like structures using the yeast three-hybrid system. Gene 710, 399-405 (2019). doi: 10.1016/j.gene.2019.06.012
34. Galy, B., Conrad, M. & Muckenthaler, M. Mechanisms controlling cellular and systemic iron homeostasis. Nat Rev Mol Cell Biol 25, 133-155 (2024). doi: 10.1038/s41580-023-00648-1
35. Wang, H. et al. FBXL5 Regulates IRP2 Stability in Iron Homeostasis via an Oxygen-Responsive [2Fe2S] Cluster. Mol Cell 78, 31-41 e35 (2020). doi: 10.1016/j.molcel.2020.02.011
36. Nishitani, Y. et al. Specific heme binding to heme regulatory motifs in iron regulatory proteins and its functional significance. J Inorg Biochem 198, 110726 (2019). doi: 10.1016/j.jinorgbio.2019.110726
37. Johnson, N. B., Deck, K. M., Nizzi, C. P. & Eisenstein, R. S. A synergistic role of IRP1 and FBXL5 proteins in coordinating iron metabolism during cell proliferation. J Biol Chem 292, 15976-15989 (2017). doi: 10.1074/jbc.M117.785741
38. Bajar, B. T. et al. Improving brightness and photostability of green and red fluorescent proteins for live cell imaging and FRET reporting. Sci Rep 6, 20889 (2016). doi: 10.1038/srep20889
39. Hentze, M. W., Muckenthaler, M. U. & Andrews, N. C. Balancing acts: molecular control of mammalian iron metabolism. Cell 117, 285-297 (2004). doi: 10.1016/s0092-8674 (04) 00343-5
40. Lagos-Quintana, M., Rauhut, R., Lendeckel, W. & Tuschl, T. Identification of novel genes coding for small expressed RNAs. Science 294, 853-858 (2001). doi: 10.1126/science. 1064921
41. Lagos-Quintana, M. et al. Identification of tissue-specific microRNAs from mouse. Curr Biol 12, 735-739 (2002). doi: 10.1016/s0960-9822 (02) 00809-6
42. Aguado, L. C. et al. RNase III nucleases from diverse kingdoms serve as antiviral effectors. Nature 547, 114-117 (2017). doi: 10.1038/nature22990
43. Shen, X. & Corey, D. R. Chemistry, mechanism and clinical status of antisense oligonucleotides and duplex RNAs. Nucleic Acids Res 46, 1584-1600 (2018). doi: 10.1093/nar/gkx1239
44. Poy, M. N. et al. A pancreatic islet-specific microRNA regulates insulin secretion. Nature 432, 226-230 (2004). doi: 10.1038/nature03076
45. Pfeifer, A. & Verma, I. M. Gene therapy: promises and problems. Annu Rev Genomics Hum Genet 2, 177-211 (2001). doi: 10.1146/annurev.genom.2.1.177
46. Kalidasan, V. et al. A guide in lentiviral vector production for hard-to-transfect cells, using cardiac-derived c-kit expressing cells as a model system. Sci Rep 11, 19265 (2021). doi: 10.1038/s41598-021-98657-7

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### Sequences Disclosed in the Present Disclosure

>IRP1
(SEQ ID No: 15)
MSNPFAHLAEPLDPVQPGKKFFNLNKLEDSRYGRLPFSIRVLLEAAIRNC
DEFLVKKQDIENILHWNVTQHKNIEVPFKPARVILQDFTGVPAVVDFAAM
RDAVKKLGGDPEKINPVCPADLVIDHSIQVDFNRRADSLQKNQDLEFERN
RERFEFLKWGSQAFHNMRIIPPGSGIIHQVNLEYLARVVFDQDGYYYPDS
LVGTDSHTTMIDGLGILGWGVGGIEAEAVMLGQPISMVLPQVIGYRLMGK
PHPLVTSTDIVLTITKHLRQVGVVGKFVEFFGPGVAQLSIADRATIANMC
PEYGATAAFFPVDEVSITYLVQTGRDEEKLKYIKKYLQAVGMFRDENDPS
QDPDFTQVVELDLKTVVPCCSGPKRPQDKVAVSDMKKDFESCLGAKQGFK
GFQVAPEHHNDHKTFIYDNTEFTLAHGSVVIAAITSCTNTSNPSVMLGAG
LLAKKAVDAGLNVMPYIKTSLSPGSGVVTYYLQESGVMPYLSQLGFDVVG
YGCMTCIGNSGPLPEPVVEAITQGDLVAVGVLSGNRNFEGRVHPNTRANY
LASPPLVIAYAIAGTIRIDFEKEPLGVNAKGQQVELKDIWPTRDEIQAVE
RQYVIPGMFKEVYQKIETVNESWNALATPSDKLFFWNSKSTYIKSPPFFE
NLTLDLQPPKSIVDAYVLLNLGDSVTTDHISPAGNIARNSPAARYLTNRG
LTPREFNSYGSRRGNDAVMARGTFANIRLLNRFLNKQAPQTIHLPSGEIL
DVFDAAERYQQAGLPLIVLAGKEYGAGSSRDWAAKGPFLLGIKAVLAESY
ERIHRSNLVGMGVIPLEYLPGENADALGLIGQERYTIIIPENLKPQMKVQ
VKLDTGKTFQAVMRFDTDVELTYFLNGGILNYMIRKMAK
>IRP2
(SEQ ID NO: 16)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDFSKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLILPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQAIENAHVLLYLGDSV
TTDHISPAGSIARNSAAAKYLINRGLTPREFNSYGARRGNDAVMTRGTFA
NIKLFNKFIGKPAPKTIHFPSGQTLDVFEAAELYQKEGIPLIILAGKKYG
SGNSRDWAAKGPYLLGVKAVLAESYEKIHKDHLIGIGIAPLQFLPGENAD
SLGLSGREIFSLTFPEELSPGITLNIQTSTGKVFSVIASFEDDVEITLYK
HGGLLNFVARKFS
>hPEST sequence (amino acid)
(SEQ ID NO: 17)
SHGFPPEVEEQAAGTLPMSCAQESGMDRHPAACASARINV
>hPEST sequence (nucleotide)
(SEQ ID NO: 18)
tctcacggctttccgcctgaggttgaagagcaagccgccggtacattgcc
tatgtcctgcgcacaagaaagcggtatggaccggcacccagccgcttgtg
cttcagctcgcatcaacgtctag
>IRP2-PEST
(SEQ ID NO: 19)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDFSKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGIDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLTKEPIALQAIENAHVLLYLGDSV
ITDHISPAGSIARNSAAAKYLINRGLTPREFNSYGARRGNDAVMTRGTFA
NIKLFNKFIGKPAPKTIHFPSGQTLDVFEAAELYQKEGIPLIILAGKKYG
SGNSRDWAAKGPYLLGVKAVLAESYEKIHKDHLIGIGIAPLQFLPGENAD
SLGLSGRETFSLTFPEELSPGITLNIQTSTGKVFSVIASFEDDVEITLYK
HGGLLNFVARKFSLSHGFPPEVEEQAAGTLPMSCAQESGMDRHPAACASA
RINV
>IRP2 Reg1 (2-318A)
(SEQ ID NO: 20)
MGCELTGSSNPFVTSIDVVLGITKHLRQVGVAGKFVEFFGSGVSQLSIVD
RITIANMCPEYGAILSFFPVDNVTLKHLEHTGFSKAKLESMETYLKAVKL
FRNDQNSSGEPEYSQVIQINLNSIVPSVSGPKRPQDRVAVTDMKSDFQAC
LNEKVGFKGFQIAAEKQKDIVSIHYEGSEYKLSHGSVVIAAVISCINNCN
PSVMLAAGLLAKKAVEAGLRVKPYIRTSLSPGSGMVTHYLSSSGVLPYLS
KLGFEIVGYGCSICVGNTAPLSDAVLNAVKQGDLVTCGILSGNKNFEGRL
CDCVRANYLASPPLVVAYAIAGTVNIDFQTEPLGTDPTGKNIYLHDIWPS
REEVHRVEEEHVILSMFKALKDKIEMGNKRWNSLEAPDSVLFPWDLKSTY
IRCPSFEDKLIKEPIALQAIENAHVLLYLGDSVTTDHISPAGSIARNSAA
AKYLTNRGLTPREFNSYGARRGNDAVMTRGTFANIKLFNKFIGKPAPKTI
HFPSGQTLDVFEAAELYQKEGIPLIILAGKKYGSGNSRDWAAKGPYLLGV
KAVLAESYEKIHKDHLIGIGIAPLQFLPGENADSLGLSGRETFSLTFPEE
LSPGITLNIQTSTGKVFSVIASFEDDVEITLYKHGGLLNFVARKFS
>IRP2 Reg2 (319-442A)
(SEQ ID NO: 21)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLILPEVVPSVSGPKRPQDRVAVTDMKSDEQACLNEKVGF
KGFQIAAEKQKDIVSIHYEGSEYKLSHGSVVIAAVISCINNCNPSVMLAA
GLLAKKAVEAGLRVKPYIRTSLSPGSGMVTHYLSSSGVLPYLSKLGFEIV
GYGCSICVGNTAPLSDAVLNAVKQGDLVTCGILSGNKNFEGRLCDCVRAN
YLASPPLVVAYAIAGTVNIDFQTEPLGTDPTGKNIYLHDIWPSREEVHRV
EEEHVILSMFKALKDKIEMGNKRWNSLEAPDSVLFPWDLKSTYIRCPSFF
DKLTKEPIALQAIENAHVLLYLGDSVTTDHISPAGSIARNSAAAKYLINR
GLIPREFNSYGARRGNDAVMTRGTFANIKLFNKFIGKPAPKTIHFPSGQT
LDVFEAAELYQKEGIPLIILAGKKYGSGNSRDWAAKGPYLLGVKAVLAES
YEKIHKDHLIGIGIAPLQFLPGENADSLGLSGRETFSLTFPEELSPGITL
NIQTSTGKVESVIASFEDDVEITLYKHGGLLNFVARKFS
>IRP2 Reg3 (443-680A)
(SEQ ID NO: 22)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGIFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVLSMFKALK
DKIEMGNKRWNSLEAPDSVLFPWDLKSTYIRCPSFFDKLIKEPIALQAIE
NAHVLLYLGDSVTTDHISPAGSIARNSAAAKYLINRGLTPREFNSYGARR
GNDAVMTRGTFANIKLFNKFIGKPAPKTIHFPSGQTLDVFEAAELYQKEG
IPLIILAGKKYGSGNSRDWAAKGPYLLGVKAVLAESYEKIHKDHLIGIGI
APLQFLPGENADSLGLSGRETFSLIFPEELSPGITLNIQTSTGKVESVIA
SFEDDVEITLYKHGGLLNFVARKFS
>IRP2 Reg4 (737-963A)
(SEQ ID NO: 23)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQA
>IRP2-900
(SEQ ID NO: 24)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGIFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVISIDVVLGITKHLRQVGVAG
KFVEFFGSGVSQLSIVDRTTIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQAIENAHVLLYLGDSV
TTDHISPAGSIARNSAAAKYLTNRGLTPREFNSYGARRGNDAVMTRGTFA
NIKLENKFIGKPAPKTIHFPSGQTLDVFEAAELYQKEGIPLIILAGKKYG
SGNSRDWAAKGPYLLGVKAVLAESYEKIHKDHLIGIGIAPLQFLPGENAD
>IRP2-850
(SEQ ID NO: 25)
MDAPKAGYAFEYLIETLNDSSHKKFFDVSKLGTKYDVLPYSIRVLLEAAV
RNCDGFLMKKEDVMNILDWKTKQSNVEVPFFPARVLLQDETGIPAMVDFA
AMREAVKTLGGDPEKVHPACPTDLTVDHSLQIDFSKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLILPEVVGCELTGSSNPFVISIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQAIENAHVLLYLGDSV
TTDHISPAGSIARNSAAAKYLTNRGLTPREFNSYGARRGNDAVMTRGTFA
NIKLFNKFIGKPAPKTIHFPSGQTLDVFEAAELYQKEGIPLIILAGKKYG
>IRP2-800
(SEQ ID NO: 26)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCINNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLTKEPIALQAIENAHVLLYLGDSV
TTDHISPAGSIARNSAAAKYLTNRGLTPREFNSYGARRGNDAVMTRGTFA
>IRP2-750
(SEQ ID NO: 27)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLTKEPIALQAIENAHVLLYLGDSV
>IRP2-700
(SEQ ID NO: 28)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGIFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLERNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCINNCNPSVMLAAGLLAKKAVEAGLRVKPYIRTSLSPGS
GMVTHYLSSSGVLPYLSKLGFEIVGYGCSICVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
>IRP2-R779E-R763D
(SEQ ID NO: 29)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGIFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITTIANMCPEYGAILSFFPVDNVTLKHLEHTG
FSKAKLESMETYLKAVKLFRNDQNSSGEPEYSQVIQINLNSIVPSVSGPK
RPQDRVAVTDMKSDFQACLNEKVGFKGFQIAAEKQKDIVSIHYEGSEYKL
SHGSVVIAAVISCINNCNPSVMLAAGLLAKKAVEAGLRVKPYIRTSLSPG
SGMVTHYLSSSGVLPYLSKLGFEIVGYGCSICVGNTAPLSDAVLNAVKQG
DLVTCGILSGNKNFEGRLCDCVRANYLASPPLVVAYAIAGTVNIDFQTEP
LGTDPTGKNIYLHDIWPSREEVHRVEEEHVILSMFKALKDKIEMGNKRWN
SLEAPDSVLFPWDLKSTYIRCPSFFDKLTKEPIALQAIENAHVLLYLGDS
VTTDHISPAGSIADNSAAAKYLTNRGLTPEEFNSYGARRGNDAVMTRGTF
ANIKLENKFIGKPAPKTIHFPSGQTLDVFEAAELYQKEGIPLIILAGKKY
GSGNSRDWAAKGPYLLGVKAVLAESYEKIHKDHLIGIGIAPLQFLPGENA
DSLGLSGRETFSLTFPEELSPGITLNIQTSTGKVFSVIASFEDDVEITLY
KHGGLLNFVARKFS
>IRP2-C578S-C581S
(SEQ ID NO: 30)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGIFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLERNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCINNCNPSVMLAAGLLAKKAVEAGLRVKPYIRTSLSPGS
GMVTHYLSSSGVLPYLSKLGFEIVGYGSSISVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQAIENAHVLLYLGDSV
TTDHISPAGSIARNSAAAKYLINRGLTPREFNSYGARRGNDAVMIRGTFA
NIKLENKFIGKPAPKTIHFPSGQTLDVFEAAELYQKEGIPLIILAGKKYG
SGNSRDWAAKGPYLLGVKAVLAESYEKIHKDHLIGIGIAPLQFLPGENAD
SLGLSGRETFSLTFPEELSPGITLNIQTSTGKVFSVIASFEDDVEITLYK
HGGLLNFVARKFS
>IRP2- C578S-C581S-R779E-R763D
(SEQ ID NO: 31)
MDAPKAGYAFEYLIETLNDSSHKKFFDVSKLGTKYDVLPYSIRVLLEAAV
RNCDGFLMKKEDVMNILDWKTKQSNVEVPFFPARVLLQDFTGIPAMVDFA
AMREAVKTLGGDPEKVHPACPTDLTVDHSLQIDESKCAIQNAPNPGGGDL
QKAGKLSPLKVQPKKLPCRGQTTCRGSCDSGELGRNSGTFSSQIENTPIL
CPFHLQPVPEPETVLKNQEVEFGRNRERLQFFKWSSRVFKNVAVIPPGTG
MAHQINLEYLSRVVFEEKDLLFPDSVVGTDSHITMVNGLGILGWGVGGIE
TEAVMLGLPVSLTLPEVVGCELTGSSNPFVTSIDVVLGITKHLRQVGVAG
KFVEFFGSGVSQLSIVDRITIANMCPEYGAILSFFPVDNVTLKHLEHTGF
SKAKLESMETYLKAVKLFRNDQNSSGEPEYSQVIQINLNSIVPSVSGPKR
PQDRVAVTDMKSDFQACLNEKVGFKGFQIAAEKQKDIVSIHYEGSEYKLS
HGSVVIAAVISCTNNCNPSVMLAAGLLAKKAVEAGLRVKPYIRTSLSPGS
GMVTHYLSSSGVLPYLSKLGFEIVGYGSSISVGNTAPLSDAVLNAVKQGD
LVTCGILSGNKNFEGRLCDCVRANYLASPPLVVAYAIAGTVNIDFQTEPL
GTDPTGKNIYLHDIWPSREEVHRVEEEHVILSMFKALKDKIEMGNKRWNS
LEAPDSVLFPWDLKSTYIRCPSFFDKLIKEPIALQAIENAHVLLYLGDSV
TTDHISPAGSIADNSAAAKYLTNRGLTPEEFNSYGARRGNDAVMTRGTFA
NIKLENKFIGKPAPKTIHFPSGQTLDVFEAAELYQKEGIPLIILAGKKYG
SGNSRDWAAKGPYLLGVKAVLAESYEKIHKDHLIGIGIAPLQFLPGENAD
SLGLSGRETFSLTFPEELSPGITLNIQTSTGKVFSVIASFEDDVEITLYK
HGGLLNFVARKFS
>L-Ferritin IRE
(SEQ ID NO: 32)
cctgcttcaacagtgcttggacgg
>HIF1A IRE
(SEQ ID NO: 33)
ttcctcggcagtgtcctgagac
>eALAS IRE
(SEQ ID NO: 34)
ttcgtcctcagtgcagggcaa
>Lin28
(SEQ ID NO: 35)
MGSVSNQQFAGGCAKAAEEAPEEAPEDAARAADEPQLLHGAGICKWENVR
MGFGFLSMTARAGVALDPPVDVFVHQSKLHMEGFRSLKEGEAVEFTEKKS
AKGLESIRVTGPGGVFCIGSERRPKGKSMQKRRSKGDRCYNCGGLDHHAK
ECKLPPQPKKCHFCQSISHMVASCPLKAQQGPSAQGKPTYFREEEEEIHS
PTLLPEAQN
>pre-let-7d
(SEQ ID NO: 36)
Gggggcttagggcggggatttcgcccacaaggaggtgccccc
>MS2
(SEQ ID NO: 37)
MASNFTQFVLVDNGGTGDVTVAPSNFANGIAEWISSNSRSQAYKVTCSVR
QSSAQNRKYTIKVEVPKGAWRSYLNMELTIPIFATNSDCELIVKAMQGLL
KDGNPIPSAIAANSGIY
>ScMS2
(SEQ ID NO: 38)
Gggagcaggtgaggatcacccatctgccacgagcgaggtgaggatcaccc
atctcgctcgtgttccc
>PP7
(SEQ ID NO: 39)
MLASKTIVLSVGEATRTLTEIQSTADRQIFEEKVGPLVGRLRLTASLRQN
GAKTAYRVNLKLDQADVVDSGLPKVRYTQVWSHDVTIVANSTEASRKSLY
DLTKSLVATSQVEDLVVNLVPLGR
>SCPP7
(SEQ ID NO: 40)
Gggagctaaggagtttatatggaaacccttagcctgctgcgtaaggagtt
tatatggaaaccettacgcagcagttccc
>U1A
(SEQ ID NO: 41)
MAAVPETRPNHTIYINNLNEKIKKDELKKSLYAIFSQFGQILDILVSRSL
KMRGQAFVIFKEVSSATNALRSMQGFPFYDKPMRIQYAKIDSDIIAKMKG
TFVERDRKREKRKPKSQETPATKKAVQGGGATPVVGAVQGPVPGMPPMTQ
APRIMHHMPGQPPYMPPPGMIPPPGLAPGQIPPGAMPPQQLMPGQMPPAQ
PLSENPPNHILFLINLPEETNELMLSMLFNQFPGFKEVRLVPGRHDIAFV
EFDNEVQAGAARDALQGFKITQNNAMKISFAKK
>Ulutr
(SEQ ID NO: 42)
Cacagcattgtacccagagtctgtccccagacattgcacctggcgctgt

1. A polynucleotide comprising:
a) a nucleotide sequence encoding an RNA-binding protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the RNA-binding protein to allow expression of the RNA-binding protein in the absence of the respective miRNA targeting said MSE sequence;
c) a first RNA element to which the RNA-binding protein specifically binds;
d) a nucleotide sequence encoding a first target polypeptide, wherein the first RNA element is positioned with respect to the nucleotide sequence encoding the first target polypeptide to allow translation of the first target polypeptide in either the presence or absence of the RNA-binding protein; and
e) optionally one or more additional combinations of an RNA element to which the RNA-binding protein specifically binds and a nucleotide sequence encoding a target polypeptide, wherein within each combination, the RNA element is positioned with respect to the nucleotide sequence encoding the target polypeptide to allow translation of the target polypeptide in either the presence or absence of the RNA-binding protein, wherein translation of each additional target polypeptide is controlled by its closest RNA element in the resulting mRNA.

2. The polynucleotide of claim 1, wherein the RNA-binding protein is selected from a ribosomal frameshift stimulator, an iron regulatory protein, Lin28, MS2 bacteriophage coat protein, PP7 bacteriophage coat protein, and U1 small nuclear ribonucleoprotein A (U1A).

3. The polynucleotide of claim 1 or 2, wherein the first RNA element is selected from a ribosomal frameshift element, an iron response element, a pre-let-7 element, an MS2 responsive RNA element, a PP7 responsive RNA element, and a U1A responsive RNA element.

4. The polynucleotide of claim 1, wherein the first RNA element to which the RNA-binding protein specifically binds comprises at least 30 nucleotides.

5. The polynucleotide of claim 4, wherein the RNA-binding protein is selected from a ribosomal frameshift stimulator, an iron regulatory protein, and Lin28.

6. The polynucleotide of claim 4 or 5, wherein the first RNA element is selected from a ribosomal frameshift element, an iron response element, and a pre-let-7 element.

7. The polynucleotide of any one of claims 1-6, wherein the one or more MSE sequences are positioned in the open reading frame (ORF) or in the 3′ untranslated region (3′ UTR) of the RNA-binding protein transcript coding sequence.

8. The polynucleotide of any one of claims 1-7, wherein the polynucleotide further comprises a first promoter, and wherein expression of the RNA-binding protein is under the control of the first promoter.

9. The polynucleotide of claim 8, wherein expression of the first target polypeptide is under the control of the first promoter.

10. The polynucleotide of claim 9, wherein the polynucleotide is structured as an alternatively spliced construct, and wherein one transcript comprises the nucleotide sequence encoding the RNA-binding protein and one or more MSE sequences, and another transcript comprises the first RNA element to which the RNA-binding protein specifically binds and the nucleotide sequence encoding the first target polypeptide.

11. The polynucleotide of claim 10, wherein the polynucleotide further comprises a 5′ donor splice site and a 3′ acceptor splice site.

12. The polynucleotide of claim 11, wherein the 5′ donor splice site comprises the sequence AG*GU(A/G)AGU or AG*GU(A/G)GAU, wherein * denotes the splice junction.

13. The polynucleotide of claim 11 or claim 12, wherein the 3′ acceptor splice site comprises a branch point, a pyrimidine tract, and the sequence AG*G, wherein * denotes the splice junction.

14. The polynucleotide of any one of claims 9-13, wherein the polynucleotide comprises one or more additional combinations of an RNA element to which the RNA-binding protein specifically binds and a nucleotide sequence encoding a target polypeptide, and wherein expression of the first target polypeptide and the one or more additional target polypeptides is under the control of the first promoter.

15. The polynucleotide of any one of claims 9-13, wherein the polynucleotide comprises one or more additional combinations of an RNA element to which the RNA-binding protein specifically binds and a nucleotide sequence encoding a target polypeptide, and wherein expression of the first target polypeptide is under the control of the first promoter, and expression of each of the additional target polypeptides is under the control of respective additional promoters.

16. The polynucleotide of claim 8, wherein the polynucleotide further comprises a second promoter, wherein expression of the first target polypeptide is under the control of the second promoter.

17. The polynucleotide of claim 16, wherein the polynucleotide comprises one or more additional combinations of an RNA element to which the RNA-binding protein specifically binds and a nucleotide sequence encoding a target polypeptide, and wherein expression of each of the additional target polypeptides is under the control of respective additional promoters.

18. The polynucleotide of claim 16, wherein the polynucleotide further comprises one or more DNA insulator elements, wherein said one or more DNA insulator elements are positioned between the 3′UTR of the RNA-binding protein transcript coding sequence and the second promoter, and wherein said DNA one or more DNA insulator elements enhance efficiency of termination of transcription of the RNA-binding protein.

19. The polynucleotide of claim 17, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

20. The polynucleotide of claim 18 or claim 19, wherein said DNA insulator element comprises CCCTC-binding factor site(s).

21. The polynucleotide of any one of claims 8-20, wherein the first promotor is an RNA polymerase II promoter.

22. The polynucleotide of claim 21, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

23. The polynucleotide of any one of claims 16-22, wherein the second promoter is an RNA polymerase II promoter.

24. The polynucleotide of claim 23, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

25. The polynucleotide of claim 9, wherein the first promoter is a bi-directional promoter which drives expression of the RNA-binding protein in one direction and the first target polypeptide in the opposite direction.

26. The polynucleotide of claim 25, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

27. The polynucleotide of any one of claim 1-26, wherein the nucleotide sequence encoding the RNA-binding protein further encodes a degron sequence.

28. The polynucleotide of claim 27, wherein the degron sequence is operably linked to the C-terminal end of the RNA-binding protein in the resulting protein.

29. The polynucleotide of claim 27, wherein the degron sequence is operably linked to the N-terminal end of the RNA-binding protein in the resulting protein.

30. The polynucleotide of any one of claims 27-29, wherein the degron sequence comprises the amino acid sequence of SEQ ID NO: 17, or a functional variant or ortholog having at least 70% sequence identity thereto.

31. A polynucleotide comprising:
a) a nucleotide sequence encoding a ribosomal frameshift stimulator;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
c) a first ribosomal frameshift element;
d) a nucleotide sequence encoding a first target polypeptide, wherein the first ribosomal frameshift element is positioned 5′ to the nucleotide sequence encoding the first target polypeptide, and wherein translation of the first target polypeptide is controlled by the first ribosomal frameshift element and requires either the presence or absence of the ribosomal frameshift stimulator; and
e) optionally one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

32. The polynucleotide of claim 31, wherein the one or more MSE sequences are positioned in the open reading frame (ORF) or in the 3′ untranslated region (3′ UTR) of the ribosomal frameshift stimulator transcript coding sequence.

33. The polynucleotide of claim 31 or claim 32, wherein the polynucleotide further comprises a first promoter, and wherein expression of the ribosomal frameshift stimulator is under the control of the first promoter.

34. The polynucleotide of claim 33, wherein expression of the first target polypeptide is under the control of the first promoter.

35. The polynucleotide of claim 34, wherein the polynucleotide is structured as an alternatively spliced construct, and wherein one transcript comprises the nucleotide sequence encoding the ribosomal frameshift stimulator and one or more MSE sequences, and another transcript comprises the first ribosomal frameshift element and the nucleotide sequence encoding the first target polypeptide.

36. The polynucleotide of claim 35, wherein the polynucleotide further comprises a 5′ donor splice site and a 3′ acceptor splice site.

37. The polynucleotide of claim 36, wherein the 5′ donor splice site comprises the sequence AG*GU(A/G)AGU or AG*GU(A/G)GAU, wherein * denotes the splice junction.

38. The polynucleotide of claim 36 or claim 37, wherein the 3′ acceptor splice site comprises a branch point, a pyrimidine tract, and the sequence AG*G, wherein * denotes the splice junction.

39. The polynucleotide of any one of claims 34-38, wherein the polynucleotide comprises one or more additional combinations of a ribosomal frameshift element and a nucleotide sequence encoding a target polypeptide, and wherein expression of the first target polypeptide and the one or more additional target polypeptides is under the control of the first promoter.

40. The polynucleotide of any one of claims 34-38, wherein the polynucleotide comprises one or more additional combinations of a ribosomal frameshift element and a nucleotide sequence encoding a target polypeptide, and wherein expression of the first target polypeptide is under the control of the first promoter, and expression of each of the additional target polypeptides is under the control of respective additional promoters.

41. The polynucleotide of claim 33, wherein the polynucleotide further comprises a second promoter, wherein expression of the first target polypeptide is under the control of the second promoter.

42. The polynucleotide of claim 41, wherein the polynucleotide comprises one or more additional combinations of a ribosomal frameshift element and a nucleotide sequence encoding a target polypeptide, and wherein expression of each of the additional target polypeptides is under the control of respective additional promoters.

43. The polynucleotide of claim 41, wherein the polynucleotide further comprises one or more DNA insulator elements, wherein said one or more DNA insulator elements are positioned between the 3′UTR of the ribosomal frameshift stimulator transcript coding sequence and the second promoter, and wherein said DNA one or more DNA insulator elements enhance efficiency of termination of transcription of the ribosomal frameshift stimulator.

44. The polynucleotide of claim 42, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

45. The polynucleotide of claim 43 or claim 44, wherein said DNA insulator element comprises CCCTC-binding factor site(s).

46. The polynucleotide of any one of claims 31-45, wherein the nucleotide sequence encoding the first target polypeptide is positioned in frame with the first ribosomal frameshift element.

47. The polynucleotide of any one of claims 31-45, wherein the nucleotide sequence encoding the first target polypeptide is positioned in-1 translation frame in relation to the first ribosomal frameshift element.

48. The polynucleotide of claim 46 or claim 47, wherein the polynucleotide comprises one or more additional combinations of a ribosomal frameshift element and a nucleotide sequence encoding a target polypeptide, and wherein the nucleotide sequence encoding each additional target polypeptide is independently positioned in frame or in-1 translation frame in relation to its closest 5′ ribosomal frameshift element in the resulting mRNA.

49. A polynucleotide comprising from 5′ to 3′:
a) a first promoter;
b) a 5′ donor splice site;
c) a nucleotide sequence encoding a ribosomal frameshift stimulator;
d) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
e) a 3′ splice acceptor site;
f) a first ribosomal frameshift element; and
g) a nucleotide sequence encoding a first target polypeptide,
wherein the first ribosomal frameshift element is positioned 5′ to the nucleotide sequence encoding the first target polypeptide,
wherein translation of the first target polypeptide is controlled by the first ribosomal frameshift element and requires either the presence or absence of the ribosomal frameshift stimulator, and
wherein the nucleotide sequence encoding the first target polypeptide is positioned in frame or in-1 translation frame in relation to the first ribosomal frameshift element.

50. The polynucleotide of claim 49, wherein the polynucleotide further comprises 3′ to the nucleotide sequence encoding the first target polypeptide from 5′ to 3′:
h) a second promoter;
i) a second ribosomal frameshift element, and
j) a nucleotide sequence encoding a second target polypeptide,
wherein the second ribosomal frameshift element is positioned 5′ to the nucleotide sequence encoding the second target polypeptide,
wherein translation of the second target polypeptide is controlled by the second ribosomal frameshift element and requires either the presence or absence of the ribosomal frameshift stimulator, and
wherein the nucleotide sequence encoding the second target polypeptide is positioned in frame or in-1 translation frame in relation to the second ribosomal frameshift element.

51. The polynucleotide of claim 50, wherein the polynucleotide further comprises a DNA insulator element, wherein said DNA insulator element is positioned between the nucleotide sequence encoding the first target polypeptide and the second promoter, and wherein said DNA insulator element enhances efficiency of termination of transcription.

52. The polynucleotide of any one of claims 49-51, wherein the 5′ donor splice site comprises the sequence AG*GU(A/G)AGU or AG*GU(A/G)GAU, wherein * denotes the splice junction.

53. The polynucleotide of any one of claims 49-52, wherein the 3′ acceptor splice site comprises a branch point, a pyrimidine tract, and the sequence AG*G, wherein * denotes the splice junction.

54. A polynucleotide comprising from 5′ to 3′:
a) a first promoter;
b) a nucleotide sequence encoding a ribosomal frameshift stimulator;
c) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence,
d) a second promoter;
e) a first ribosomal frameshift element, and
f) a nucleotide sequence encoding a first target polypeptide,
wherein the first ribosomal frameshift element is positioned 5′ to the nucleotide sequence encoding the first target polypeptide,
wherein translation of the first target polypeptide is controlled by the first ribosomal frameshift element and requires either the presence or absence of the ribosomal frameshift stimulator, and
wherein the nucleotide sequence encoding the first target polypeptide is positioned in frame or in-1 translation frame in relation to the first ribosomal frameshift element.

55. The polynucleotide of claim 54, wherein the polynucleotide further comprises a DNA insulator element, wherein said DNA insulator element is positioned between the 3′UTR of the ribosomal frameshift stimulator transcript coding sequence and the second promoter, and wherein said DNA insulator element enhances efficiency of termination of transcription.

56. The polynucleotide of claim 54 or claim 55, wherein the polynucleotide further comprises 3′ to the nucleotide sequence encoding the first target polypeptide from 5′ to 3′:
g) a third promoter;
h) a second ribosomal frameshift element, and
i) a nucleotide sequence encoding a second target polypeptide,
wherein the second ribosomal frameshift element is positioned 5′ to the nucleotide sequence encoding the second target polypeptide,
wherein translation of the second target polypeptide is controlled by the second ribosomal frameshift element and requires either the presence or absence of the ribosomal frameshift stimulator, and
wherein the nucleotide sequence encoding the second target polypeptide is positioned in frame or in-1 translation frame in relation to the second ribosomal frameshift element.

57. The polynucleotide of claim 56, wherein the polynucleotide further comprises a DNA insulator element, wherein said DNA insulator element is positioned between the nucleotide sequence encoding the first target polypeptide and the third promoter, and wherein said DNA insulator element enhances efficiency of termination of transcription.

58. The polynucleotide of any one of claim 51, 55, or 57, wherein said DNA insulator element comprises CCCTC-binding factor site(s).

59. The polynucleotide of any one of claims 33-58, wherein the first promotor is an RNA polymerase II promoter.

60. The polynucleotide of claim 59, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

61. The polynucleotide of any one of claims 41-45 and 50-60, wherein the second promoter is an RNA polymerase II promoter.

62. The polynucleotide of claim 61, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

63. The polynucleotide of claim 56 or claim 57, wherein the third promoter is an RNA polymerase II promoter.

64. The polynucleotide of claim 63, wherein the third promoter is a cytomegalovirus (CMV) promoter, a simian virus 40 (SV40) promoter, a human elongation factor 1 alpha (EF1α) promoter, or a phosphoglycerate kinase (PGK) promoter.

65. The polynucleotide of claim 34, wherein the first promoter is a bi-directional promoter which drives expression of the ribosomal frameshift stimulator in one direction and the first target polypeptide in the opposite direction.

66. A polynucleotide comprising from 5′ to 3′:
a) one or more microRNA (miRNA) Silencing Element (MSE) sequences, wherein each of said one or more MSE sequences is targeted by a miRNA,
b) a nucleotide sequence encoding a ribosomal frameshift stimulator, wherein each of said one or more MSE sequences is positioned with respect to the nucleotide sequence encoding the ribosomal frameshift stimulator to allow expression of the ribosomal frameshift stimulator in the absence of the respective miRNA targeting said MSE sequence;
c) a bi-directional promoter;
d) a first ribosomal frameshift element;
e) a nucleotide sequence encoding a first target polypeptide, wherein the first ribosomal frameshift element is positioned 5′ to the nucleotide sequence encoding the first target polypeptide, wherein translation of the first target polypeptide is controlled by the first ribosomal frameshift element and requires either the presence or absence of the ribosomal frameshift stimulator, and wherein the bi-directional promoter drives expression of the ribosomal frameshift stimulator in one direction and the first target polypeptide in the opposite direction; and
f) optionally one or more additional combinations of a ribosomal frameshift element and a nucleotide sequence encoding a target polypeptide, wherein within each combination, the ribosomal frameshift element is positioned 5′ to the nucleotide sequence encoding the target polypeptide, wherein translation of each additional target polypeptide is controlled by its closest 5′ ribosomal frameshift element in the resulting mRNA and independently requires either the presence or absence of the ribosomal frameshift stimulator.

67. The polynucleotide of claim 65 or claim 66, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

68. The polynucleotide of any one of claims 2-3, 5-67, wherein the ribosomal frameshift stimulator is a cardiovirus 2A protein, or a functional variant or ortholog thereof.

69. The polynucleotide of claim 68, wherein the cardiovirus 2A protein comprises the amino acid sequence
MSPNALDISRTYPTLHVLIQFNHRGLEVRLFRHGHFWAETRADVILRSKTKQV SFLSNGNYPSMDSRAPWNPWKNTYQAVLRAEPCRVTMDIYYKRVRPFRLPL VQKEWPVREENVFGLYRIFNAHYAGYFADLLIHDIETNPG (SEQ ID NO: 1), or a variant thereof.

70. The polynucleotide of claim 69, wherein the cardiovirus 2A protein consists of the amino acid sequence
MSPNALDISRTYPTLHVLIQFNHRGLEVRLFRHGHFWAETRADVILRSKTKQV SFLSNGNYPSMDSRAPWNPWKNTYQAVLRAEPCRVTMDIYYKRVRPFRLPL VQKEWPVREENVFGLYRIFNAHYAGYFADLLIHDIETNPG (SEQ ID NO: 1), or a variant thereof.

71. The polynucleotide of claim 68, wherein the nucleotide sequence encoding the cardiovirus 2A protein comprises the sequence
ATGAGTCCAAATGCCCTAGACATTTCAAGAACATACCCCACGTTACATGT TCTCATTCAATTCAACCATAGAGGTTTGGAGGTTAGATTGTTTAGACATGG ACACTTTTGGGCTGAAACACGTGCGGACGTGATTCTGAGATCAAAGACCA AACAGGTCTCTTTCCTGAGCAACGGGAACTACCCGTCAATGGACTCTAGA GCTCCCTGGAATCCTTGGAAGAATACCTACCAGGCGGTTCTAAGAGCAGA ACCATGTAGAGTGACCATGGATATATATTATAAGAGAGTCAGGCCTTTTA GACTGCCCCTGGTTCAGAAGGAATGGCCCGTGCGAGAGGAGAACGTTTTC GGTTTGTACCGGATCTTCAATGCCCACTACGCTGGTTACTTTGCGGACCTA CTGATTCATGACATTGAGACAAATCCAGGGTAG (SEQ ID NO: 2), or a variant thereof.

72. The polynucleotide of claim 71, wherein the nucleotide sequence encoding the cardiovirus 2A protein consists of the sequence
ATGAGTCCAAATGCCCTAGACATTTCAAGAACATACCCCACGTTACATGT TCTCATTCAATTCAACCATAGAGGTTTGGAGGTTAGATTGTTTAGACATGG ACACTTTTGGGCTGAAACACGTGCGGACGTGATTCTGAGATCAAAGACCA AACAGGTCTCTTTCCTGAGCAACGGGAACTACCCGTCAATGGACTCTAGA GCTCCCTGGAATCCTTGGAAGAATACCTACCAGGCGGTTCTAAGAGCAGA ACCATGTAGAGTGACCATGGATATATATTATAAGAGAGTCAGGCCTTTTA GACTGCCCCTGGTTCAGAAGGAATGGCCCGTGCGAGAGGAGAACGTTTTC GGTTTGTACCGGATCTTCAATGCCCACTACGCTGGTTACTTTGCGGACCTA CTGATTCATGACATTGAGACAAATCCAGGGTAG (SEQ ID NO: 2), or a variant thereof.

73. The polynucleotide of any one of claim 2-3, 5-72, wherein the nucleotide sequence encoding a ribosomal frameshift stimulator further encodes a degron sequence.

74. The polynucleotide of claim 73, wherein the degron sequence is operably linked to the C-terminal end of the ribosomal frameshift stimulator in the resulting protein.

75. The polynucleotide of claim 73, wherein the degron sequence is operably linked to the N-terminal end of the ribosomal frameshift stimulator in the resulting protein.

76. The polynucleotide of any one of claims 73-75, wherein the degron sequence comprises the amino acid sequence of SEQ ID NO: 17, or a functional variant having at least 70% sequence identity thereto.

77. The polynucleotide of any one of claims 3, 6-76, wherein the first ribosomal frameshift element comprises from 5′ to 3′:
1) a 5′ shift site,
2) a spacer sequence, and
3) a stem-loop structure.

78. The polynucleotide of any one of claims 50-53, 56 and 57, wherein the second ribosomal frameshift element comprises from 5′ to 3′:
1) a 5′ shift site,
2) a spacer sequence, and
3) a stem-loop structure.

79. The polynucleotide of claim 77 or claim 78, wherein the 5′ shift site comprises the sequence 5′-GGUUUUU-3′.

80. The polynucleotide of claim 79, wherein the 5′ shift site consists of the sequence 5′-GGUUUUU-3′.

81. The polynucleotide of any one of claims 77-80, wherein the spacer sequence is 13 or 14 nucleotides long.

82. The polynucleotide of claim 81, wherein the spacer sequence comprises the sequence CAGACUCAAGGAG (SEQ ID NO: 4).

83. The polynucleotide of claim 82, wherein the spacer sequence consists of the sequence CAGACUCAAGGAG (SEQ ID NO: 4).

84. The polynucleotide of any one of claims 77-83, wherein the stem-loop structure comprises a stem of 7 nucleotides.

85. The polynucleotide of claim 84, wherein the stem-loop structure comprises the sequence 5′-CGGCAGU-3′ followed by a 21-nucleotide loop and the complementary sequence 5′ACUGCCG-3′.

86. The polynucleotide of any one of claims 77-85, wherein the loop of the stem-loop structure comprises an adenosine triplet followed by a cytosine triplet.

87. The polynucleotide of claim 85 or claim 86, wherein the loop structure comprises the sequence GUCAUCAAUGGCUCAAACCCU (SEQ ID NO: 7).

88. The polynucleotide of claim 87, wherein the loop structure consists of the sequence GUCAUCAAUGGCUCAAACCCU (SEQ ID NO: 7).

89. The polynucleotide of any one of claims 3, 6-88, wherein the first ribosomal frameshift element comprises the sequence GGUUUUUCAGACUCAAGGAGCGGCAGUGUCAUCAAUGGCUCAAACCCU ACUGCCG (SEQ ID NO: 8), or a sequence having at least 60% identity thereto.

90. The polynucleotide of any one of claims 50-53, 56, 57, and 78-89 wherein the second ribosomal frameshift element comprises the sequence GGUUUUUCAGACUCAAGGAGCGGCAGUGUCAUCAAUGGCUCAAACCCU ACUGCCG (SEQ ID NO: 8), or a sequence having at least 60% identity thereto.

91. The polynucleotide of any one of claims 3, 6-90, wherein the first ribosomal frameshift element causes a-1 frameshift in the presence of the ribosomal frameshift stimulator.

92. The polynucleotide of any one of claims 50-53, 56, 57, and 78-91, wherein the second ribosomal frameshift element causes a-1 frameshift in the presence of the ribosomal frameshift stimulator.

93. The polynucleotide of any one of claims 31-92, wherein the polynucleotide further comprises one or more additional combinations of an additional ribosomal frameshift element and a nucleotide sequence encoding an additional target polypeptide, wherein the additional ribosomal frameshift element is positioned 5′ to the nucleotide sequence encoding the additional target polypeptide,
wherein translation of the additional target polypeptide is controlled by the additional ribosomal frameshift element and requires either the presence or absence of the ribosomal frameshift stimulator, and
wherein the nucleotide sequence encoding the additional target polypeptide is positioned in frame or in-1 translation frame in relation to the additional ribosomal frameshift element.

94. A polynucleotide comprising:
a) a nucleotide sequence encoding an iron regulatory protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence;
c) a first iron response element;
d) a nucleotide sequence encoding a first target polypeptide, wherein the first iron response element is positioned with respect to the nucleotide sequence encoding the first target polypeptide to allow translation of the first target polypeptide in the absence of the iron regulatory protein; and
e) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

95. The polynucleotide of claim 94, wherein the one or more MSE sequences are positioned in the open reading frame (ORF) or in the 3′ untranslated region (3′ UTR) of the iron regulatory protein transcript coding sequence.

96. The polynucleotide of claim 94 or claim 95, wherein the polynucleotide further comprises a first promoter, and wherein expression of the iron regulatory protein is under the control of the first promoter.

97. The polynucleotide of claim 96, wherein expression of the first target polypeptide is under the control of the first promoter.

98. The polynucleotide of claim 97, wherein the polynucleotide is structured as an alternatively spliced construct, and wherein one transcript comprises the nucleotide sequence encoding the iron regulatory protein and one or more MSE sequences, and another transcript comprises the first iron response element and the nucleotide sequence encoding the first target polypeptide.

99. The polynucleotide of claim 98, wherein the polynucleotide further comprises a 5′ donor splice site and a 3′ acceptor splice site.

100. The polynucleotide of claim 99, wherein the 5′ donor splice site comprises the sequence AG*GU(A/G)AGU or AG*GU(A/G)GAU, wherein * denotes the splice junction.

101. The polynucleotide of claim 99 or claim 100, wherein the 3′ acceptor splice site comprises a branch point, a pyrimidine tract, and the sequence AG*G, wherein * denotes the splice junction.

102. The polynucleotide of any one of claims 94-101, wherein the polynucleotide comprises one or more additional combinations of an iron response element and a nucleotide sequence encoding a target polypeptide, and wherein expression of the first target polypeptide and the one or more additional target polypeptides is under the control of the first promoter.

103. The polynucleotide of any one of claims 94-101, wherein the polynucleotide comprises one or more additional combinations of an iron response element and a nucleotide sequence encoding a target polypeptide, and wherein expression of the first target polypeptide is under the control of the first promoter, and expression of each of the additional target polypeptides is under the control of respective additional promoters.

104. The polynucleotide of claim 96, wherein the polynucleotide further comprises a second promoter, wherein expression of the first target polypeptide is under the control of the second promoter.

105. The polynucleotide of claim 104, wherein the polynucleotide comprises one or more additional combinations of an iron response element and a nucleotide sequence encoding a target polypeptide, and wherein expression of each of the additional target polypeptides is under the control of respective additional promoters.

106. The polynucleotide of claim 104, wherein the polynucleotide further comprises one or more DNA insulator elements, wherein said one or more DNA insulator elements are positioned between the 3′UTR of the iron regulatory protein transcript coding sequence and the second promoter, and wherein said DNA one or more DNA insulator elements enhance efficiency of termination of transcription of the iron regulatory protein.

107. The polynucleotide of claim 105, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

108. The polynucleotide of claim 106 or claim 107, wherein said DNA insulator element comprises CCCTC-binding factor site(s).

109. A polynucleotide comprising from 5′ to 3′:
a) a first promoter;
b) a 5′ donor splice site;
c) a nucleotide sequence encoding an iron regulatory protein;
d) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence;
e) a 3′ splice acceptor site;
f) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
g) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

110. The polynucleotide of claim 109, wherein the 5′ donor splice site comprises the sequence AG*GU(A/G)AGU or AG*GU(A/G)GAU, wherein * denotes the splice junction.

111. The polynucleotide of claim 109 or 110, wherein the 3′ acceptor splice site comprises a branch point, a pyrimidine tract, and the sequence AG*G, wherein * denotes the splice junction.

112. A polynucleotide comprising from 5′ to 3′:
a) a first promoter;
b) a nucleotide sequence encoding an iron regulatory protein;
c) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the iron regulatory protein to allow expression of the iron regulatory protein in the absence of the respective miRNA targeting said MSE sequence,
d) a second promoter;
e) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
f) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

113. The polynucleotide of claim 112, wherein the polynucleotide further comprises a DNA insulator element, wherein said DNA insulator element is positioned between the 3′UTR of the iron regulatory protein transcript coding sequence and the second promoter, and wherein said DNA insulator element enhances efficiency of termination of transcription.

114. The polynucleotide of claim 113, wherein said DNA insulator element comprises CCCTC-binding factor site(s).

115. The polynucleotide of any one of claims 96-114, wherein the first promotor is an RNA polymerase II promoter.

116. The polynucleotide of claim 115, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

117. The polynucleotide of any one of claims 104-108 and 112-116, wherein the second promoter is an RNA polymerase II promoter.

118. The polynucleotide of claim 117, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

119. The polynucleotide of claim 97, wherein the first promoter is a bi-directional promoter which drives expression of the iron regulatory protein in one direction and the first target polypeptide in the opposite direction.

120. A polynucleotide comprising from 5′ to 3′:
a) one or more microRNA (miRNA) Silencing Element (MSE) sequences, wherein each of said one or more MSE sequences is targeted by a miRNA;
b) a nucleotide sequence encoding an iron regulatory protein, wherein each of said one or more MSE sequences is positioned with respect to the nucleotide sequence encoding the iron regulatory protein to allow expression of the iron regulatory protein in the absence of the respective miRNA targeting said MSE sequence;
c) a bi-directional promoter;
d) a first iron response element and a nucleotide sequence encoding a first target polypeptide, wherein the first iron response element is positioned with respect to the nucleotide sequence encoding the first target polypeptide to allow translation of the first target polypeptide in the absence of the iron regulatory protein; and
e) optionally one or more additional combinations of an iron response element and a nucleotide sequence encoding a target polypeptide, wherein within each combination, the iron response element is positioned with respect to the nucleotide sequence encoding the target polypeptide to allow translation of the target polypeptide in the absence of the iron regulatory protein, wherein translation of each additional target polypeptide is controlled by its closest iron response element in the resulting mRNA.

121. The polynucleotide of claim 119 or 120, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

122. The polynucleotide of any one of claims 2-3, 5-30, 94-121, wherein the iron regulatory protein is Iron Regulatory Protein 2 (IRP2), Iron Regulatory Protein 1 (IRP1), or a functional variant or ortholog thereof.

123. The polynucleotide of claim 122, wherein the iron regulatory protein is human IRP2, or a functional variant or ortholog thereof.

124. The polynucleotide of claim 123, wherein the human IRP2 comprises the amino acid sequence of SEQ ID NO: 16, or a sequence having at least 70% sequence identity thereto.

125. The polynucleotide of claim 123, wherein the human IRP2 variant comprises the amino acid sequence of any one of SEQ ID NOs: 21, 24, 25, 26, 27, and 30, or a sequence having at least 70% sequence identity thereto.

126. The polynucleotide of claim 123, wherein the human IRP2 variant comprises or consists essentially of the amino acids 1-318 and 443-750 of SEQ ID NO: 16.

127. The polynucleotide of claim 123, wherein the human IRP2 variant comprises a substitution at one or more positions selected from cysteine 120, cysteine 375, cysteine 578, and cysteine 581.

128. The polynucleotide of claim 122, wherein the iron regulatory protein is human IRP1, or a functional variant or ortholog thereof.

129. The polynucleotide of claim 128, wherein the human IRP1 comprises the amino acid sequence of SEQ ID NO: 15, or a sequence having at least 70% sequence identity thereto.

130. The polynucleotide of any one of claim 94-129, wherein the nucleotide sequence encoding an iron regulatory protein further encodes a degron sequence.

131. The polynucleotide of claim 130, wherein the degron sequence is operably linked to the C-terminal end of the iron regulatory protein in the resulting protein.

132. The polynucleotide of claim 130, wherein the degron sequence is operably linked to the N-terminal end of the iron regulatory protein in the resulting protein.

133. The polynucleotide of any one of claims 130-132, wherein the degron sequence comprises the amino acid sequence of SEQ ID NO: 17, or a functional variant or ortholog having at least 70% sequence identity thereto.

134. The polynucleotide of any one of claims 94-133, wherein the first iron response element is positioned in the 5′ UTR or the 3′ UTR of the nucleotide sequence encoding a first target polypeptide.

135. The polynucleotide of any one of claims 94-134, wherein when one or more additional combinations of an iron response element and a nucleotide sequence encoding a target polypeptide are present, each additional iron response element is positioned independently in the 5′ UTR or the 3′ UTR of the nucleotide sequence encoding the respective additional target polypeptide.

136. The polynucleotide of any one of claims 3, 6-30, and 94-135, wherein the iron response element comprises a stem-loop element comprising a 6-nucleotide apical loop motif 5′-CAGWGH-3′ (W=A or U and H=A, C, or U) operably linked to a five base-pair stem.

137. The polynucleotide of claim 136, wherein the iron response element further comprises an internal bulge containing a conserved cytosine (C) operably linked to the five base-pair stem.

138. The polynucleotide of any one of claims 3, 6-30, and 94-137, wherein the iron response element comprises an iron response element found in the 5′ UTR of the human ferritin transcript, hypoxia inducible factor 1 subunit alpha (HIFIA) transcript, or erythroid 5-aminolevulinate synthase (eALAS) transcript, or a functional variant or ortholog thereof.

139. The polynucleotide of any one of claims 3, 6-30, and 94-138, wherein the iron response element comprises the nucleotide sequence of any one of SEQ ID Nos: 32, 33, or 34, or a sequence having at least 70% sequence identity thereto.

140. A polynucleotide comprising:
a) a nucleotide sequence encoding Lin28;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding Lin28 to allow expression of Lin28 in the absence of the respective miRNA targeting said MSE sequence;
c) a first pre-let-7 element;
d) a nucleotide sequence encoding a first target polypeptide, wherein the first pre-let-7 element is positioned with respect to the nucleotide sequence encoding the first target polypeptide to allow translation of the first target polypeptide in the absence of Lin28; and
e) optionally one or more additional combinations of a pre-let-7 element and a nucleotide sequence encoding a target polypeptide, wherein within each combination, the pre-let-7 element is positioned with respect to the nucleotide sequence encoding the target polypeptide to allow translation of the target polypeptide in the absence of Lin28, wherein translation of each additional target polypeptide is controlled by its closest pre-let-7 element in the resulting mRNA.

141. The polynucleotide of claim 140, wherein the one or more MSE sequences are positioned in the open reading frame (ORF) or in the 3′ untranslated region (3′ UTR) of the Lin28 transcript coding sequence.

142. The polynucleotide of claim 140 or claim 141, wherein the polynucleotide further comprises a first promoter, and wherein expression of Lin28 is under the control of the first promoter.

143. The polynucleotide of claim 142, wherein expression of the first target polypeptide is under the control of the first promoter.

144. The polynucleotide of claim 143, wherein the polynucleotide is structured as an alternatively spliced construct, and wherein one transcript comprises the nucleotide sequence encoding Lin28 and one or more MSE sequences, and another transcript comprises the first pre-let-7 element and the nucleotide sequence encoding the first target polypeptide.

145. The polynucleotide of claim 144, wherein the polynucleotide further comprises a 5′ donor splice site and a 3′ acceptor splice site.

146. The polynucleotide of claim 145, wherein the 5′ donor splice site comprises the sequence AG*GU(A/G)AGU or AG*GU(A/G)GAU, wherein * denotes the splice junction.

147. The polynucleotide of claim 145 or claim 146, wherein the 3′ acceptor splice site comprises a branch point, a pyrimidine tract, and the sequence AG*G, wherein * denotes the splice junction.

148. The polynucleotide of any one of claims 143-147, wherein the polynucleotide comprises one or more additional combinations of a pre-let-7 element and a nucleotide sequence encoding a target polypeptide, and wherein expression of the first target polypeptide and the one or more additional target polypeptides is under the control of the first promoter.

149. The polynucleotide of any one of claims 143-147, wherein the polynucleotide comprises one or more additional combinations of a pre-let-7 element and a nucleotide sequence encoding a target polypeptide, and wherein expression of the first target polypeptide is under the control of the first promoter, and expression of each of the additional target polypeptides is under the control of respective additional promoters.

150. The polynucleotide of claim 142, wherein the polynucleotide further comprises a second promoter, wherein expression of the first target polypeptide is under the control of the second promoter.

151. The polynucleotide of claim 150, wherein the polynucleotide comprises one or more additional combinations of a pre-let-7 element and a nucleotide sequence encoding a target polypeptide, and wherein expression of each of the additional target polypeptides is under the control of respective additional promoters.

152. The polynucleotide of claim 150, wherein the polynucleotide further comprises one or more DNA insulator elements, wherein said one or more DNA insulator elements are positioned between the 3′UTR of Lin28 transcript coding sequence and the second promoter, and wherein said DNA one or more DNA insulator elements enhance efficiency of termination of transcription of the Lin28.

153. The polynucleotide of claim 151, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

154. The polynucleotide of claim 152 or claim 153, wherein said DNA insulator element comprises CCCTC-binding factor site(s).

155. The polynucleotide of any one of claims 142-154, wherein the first promotor is an RNA polymerase II promoter.

156. The polynucleotide of claim 155, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

157. The polynucleotide of any one of claims 150-156, wherein the second promoter is an RNA polymerase II promoter.

158. The polynucleotide of claim 157, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

159. The polynucleotide of claim 143, wherein the first promoter is a bi-directional promoter which drives expression of Lin28 in one direction and the first target polypeptide in the opposite direction.

160. The polynucleotide of claim 159, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

161. The polynucleotide of any one of claims 2-3, 5-30, and 140-160, wherein Lin28 comprises the amino acid sequence of SEQ ID NO: 35, or a sequence having at least 70% sequence identity thereto.

162. The polynucleotide of any one of claim 140-161, wherein the nucleotide sequence encoding Lin28 further encodes a degron sequence.

163. The polynucleotide of claim 162, wherein the degron sequence is operably linked to the C-terminal end of Lin28 in the resulting protein.

164. The polynucleotide of claim 162, wherein the degron sequence is operably linked to the N-terminal end of Lin28 in the resulting protein.

165. The polynucleotide of any one of claims 162-164, wherein the degron sequence comprises the amino acid sequence of SEQ ID NO: 17, or a functional variant or ortholog having at least 70% sequence identity thereto.

166. The polynucleotide of any one of claims 140-165, wherein the first pre-let-7 element is positioned in the 5′ UTR or the 3′ UTR of the nucleotide sequence encoding a first target polypeptide.

167. The polynucleotide of any one of claims 140-165, wherein when one or more additional combinations of a pre-let-7 element and a nucleotide sequence encoding a target polypeptide are present, each additional pre-let-7 element is positioned independently in the 5′ UTR or the 3′ UTR of the nucleotide sequence encoding the respective additional target polypeptide.

168. The polynucleotide of any one of claims 3, 6-30, and 140-167, wherein the pre-let-7 element comprises a tetranucleotide motif 5′-GGAG-3′.

169. The polynucleotide of any one of claims 3, 6-30, and 140-168, wherein the pre-let-7 element comprises the nucleotide sequence of SEQ ID No: 36, or a sequence having at least 70% sequence identity thereto.

170. A polynucleotide comprising:
a) a nucleotide sequence encoding an MS2 bacteriophage coat protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the MS2 bacteriophage coat protein to allow expression of the MS2 bacteriophage coat protein in the absence of the respective miRNA targeting said MSE sequence;
c) a first MS2 responsive RNA element;
d) a nucleotide sequence encoding a first target polypeptide, wherein the first MS2 responsive RNA element is positioned with respect to the nucleotide sequence encoding the first target polypeptide to allow translation of the first target polypeptide in the absence of the MS2 bacteriophage coat protein; and
e) optionally one or more additional combinations of an MS2 responsive RNA element and a nucleotide sequence encoding a target polypeptide, wherein within each combination, the MS2 responsive RNA element is positioned with respect to the nucleotide sequence encoding the target polypeptide to allow translation of the target polypeptide in the absence of the MS2 bacteriophage coat protein, wherein translation of each additional target polypeptide is controlled by its closest MS2 responsive RNA element in the resulting mRNA.

171. The polynucleotide of claim 170, wherein the one or more MSE sequences are positioned in the open reading frame (ORF) or in the 3′ untranslated region (3′ UTR) of the MS2 transcript coding sequence.

172. The polynucleotide of claim 170 or claim 171, wherein the polynucleotide further comprises a first promoter, and wherein expression of the MS2 bacteriophage coat protein is under the control of the first promoter.

173. The polynucleotide of claim 172, wherein expression of the first target polypeptide is under the control of the first promoter.

174. The polynucleotide of claim 173, wherein the polynucleotide is structured as an alternatively spliced construct, and wherein one transcript comprises the nucleotide sequence encoding the MS2 bacteriophage coat protein and one or more MSE sequences, and another transcript comprises the first MS2 responsive RNA element and the nucleotide sequence encoding the first target polypeptide.

175. The polynucleotide of claim 174, wherein the polynucleotide further comprises a 5′ donor splice site and a 3′ acceptor splice site.

176. The polynucleotide of claim 175, wherein the 5′ donor splice site comprises the sequence AG*GU(A/G)AGU or AG*GU(A/G)GAU, wherein * denotes the splice junction.

177. The polynucleotide of claim 175 or claim 176, wherein the 3′ acceptor splice site comprises a branch point, a pyrimidine tract, and the sequence AG*G, wherein * denotes the splice junction.

178. The polynucleotide of any one of claims 170-177, wherein the polynucleotide comprises one or more additional combinations of an MS2 responsive RNA element and a nucleotide sequence encoding a target polypeptide, and wherein expression of the first target polypeptide and the one or more additional target polypeptides is under the control of the first promoter.

179. The polynucleotide of any one of claims 170-177, wherein the polynucleotide comprises one or more additional combinations of an MS2 responsive RNA element and a nucleotide sequence encoding a target polypeptide, and wherein expression of the first target polypeptide is under the control of the first promoter, and expression of each of the additional target polypeptides is under the control of respective additional promoters.

180. The polynucleotide of claim 172, wherein the polynucleotide further comprises a second promoter, wherein expression of the first target polypeptide is under the control of the second promoter.

181. The polynucleotide of claim 180, wherein the polynucleotide comprises one or more additional combinations of an MS2 responsive RNA element and a nucleotide sequence encoding a target polypeptide, and wherein expression of each of the additional target polypeptides is under the control of respective additional promoters.

182. The polynucleotide of claim 180, wherein the polynucleotide further comprises one or more DNA insulator elements, wherein said one or more DNA insulator elements are positioned between the 3′UTR of the MS2 bacteriophage coat protein transcript coding sequence and the second promoter, and wherein said DNA one or more DNA insulator elements enhance efficiency of termination of transcription of the MS2 bacteriophage coat protein.

183. The polynucleotide of claim 181, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

184. The polynucleotide of claim 182 or claim 183, wherein said DNA insulator element comprises CCCTC-binding factor site(s).

185. The polynucleotide of any one of claims 182-184, wherein the first promotor is an RNA polymerase II promoter.

186. The polynucleotide of claim 185, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

187. The polynucleotide of any one of claims 180-186, wherein the second promoter is an RNA polymerase II promoter.

188. The polynucleotide of claim 187, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

189. The polynucleotide of claim 173, wherein the first promoter is a bi-directional promoter which drives expression of the MS2 bacteriophage coat protein in one direction and the first target polypeptide in the opposite direction.

190. The polynucleotide of claim 189, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

191. The polynucleotide of any one of claims 2-3, 7-30, and 170-190, wherein the MS2 bacteriophage coat protein comprises the amino acid sequence of SEQ ID NO: 37, or a sequence having at least 70% sequence identity thereto.

192. The polynucleotide of any one of claims 170-191, wherein the nucleotide sequence encoding the MS2 bacteriophage coat protein further encodes a degron sequence.

193. The polynucleotide of claim 192, wherein the degron sequence is operably linked to the C-terminal end of the MS2 bacteriophage coat protein in the resulting protein.

194. The polynucleotide of claim 192, wherein the degron sequence is operably linked to the N-terminal end of the MS2 bacteriophage coat protein in the resulting protein.

195. The polynucleotide of any one of claims 192-194, wherein the degron sequence comprises the amino acid sequence of SEQ ID NO: 17, or a functional variant or ortholog having at least 70% sequence identity thereto.

196. The polynucleotide of any one of claims 170-195, wherein the first MS2 responsive RNA element is positioned in the 5′ UTR or the 3′ UTR of the nucleotide sequence encoding a first target polypeptide.

197. The polynucleotide of any one of claims 170-196, wherein when one or more additional combinations of a MS2 responsive RNA element and a nucleotide sequence encoding a target polypeptide are present, each additional MS2 responsive RNA element is positioned independently in the 5′ UTR or the 3′ UTR of the nucleotide sequence encoding the respective additional target polypeptide.

198. The polynucleotide of any one of claims 3, 7-30, and 170-197, wherein the MS2 responsive RNA element comprises the nucleotide sequence of SEQ ID No: 38, or a sequence having at least 70% sequence identity thereto.

199. A polynucleotide comprising:
a) a nucleotide sequence encoding a PP7 bacteriophage coat protein;
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the PP7 bacteriophage coat protein to allow expression of the PP7 bacteriophage coat protein in the absence of the respective miRNA targeting said MSE sequence;
c) a first PP7 responsive RNA element;
d) a nucleotide sequence encoding a first target polypeptide, wherein the first PP7 responsive RNA element is positioned with respect to the nucleotide sequence encoding the first target polypeptide to allow translation of the first target polypeptide in the absence of the PP7 bacteriophage coat protein; and
e) optionally one or more additional combinations of a PP7 responsive RNA element and a nucleotide sequence encoding a target polypeptide, wherein within each combination, the PP7 responsive RNA element is positioned with respect to the nucleotide sequence encoding the target polypeptide to allow translation of the target polypeptide in the absence of the PP7 bacteriophage coat protein, wherein translation of each additional target polypeptide is controlled by its closest PP7 responsive RNA element in the resulting mRNA.

200. The polynucleotide of claim 199, wherein the one or more MSE sequences are positioned in the open reading frame (ORF) or in the 3′ untranslated region (3′ UTR) of the PP7 transcript coding sequence.

201. The polynucleotide of claim 199 or claim 200, wherein the polynucleotide further comprises a first promoter, and wherein expression of the PP7 bacteriophage coat protein is under the control of the first promoter.

202. The polynucleotide of claim 201, wherein expression of the first target polypeptide is under the control of the first promoter.

203. The polynucleotide of claim 202, wherein the polynucleotide is structured as an alternatively spliced construct, and wherein one transcript comprises the nucleotide sequence encoding the PP7 bacteriophage coat protein and one or more MSE sequences, and another transcript comprises the first PP7 responsive RNA element and the nucleotide sequence encoding the first target polypeptide.

204. The polynucleotide of claim 203, wherein the polynucleotide further comprises a 5′ donor splice site and a 3′ acceptor splice site.

205. The polynucleotide of claim 204, wherein the 5′ donor splice site comprises the sequence AG*GU(A/G)AGU or AG*GU(A/G)GAU, wherein * denotes the splice junction.

206. The polynucleotide of claim 204 or claim 205, wherein the 3′ acceptor splice site comprises a branch point, a pyrimidine tract, and the sequence AG*G, wherein * denotes the splice junction.

207. The polynucleotide of any one of claims 202-206, wherein the polynucleotide comprises one or more additional combinations of a PP7 responsive RNA element and a nucleotide sequence encoding a target polypeptide, and wherein expression of the first target polypeptide and the one or more additional target polypeptides is under the control of the first promoter.

208. The polynucleotide of any one of claims 202-207, wherein the polynucleotide comprises one or more additional combinations of a PP7 responsive RNA element and a nucleotide sequence encoding a target polypeptide, and wherein expression of the first target polypeptide is under the control of the first promoter, and expression of each of the additional target polypeptides is under the control of respective additional promoters.

209. The polynucleotide of claim 201, wherein the polynucleotide further comprises a second promoter, wherein expression of the first target polypeptide is under the control of the second promoter.

210. The polynucleotide of claim 209, wherein the polynucleotide comprises one or more additional combinations of a PP7 responsive RNA element and a nucleotide sequence encoding a target polypeptide, and wherein expression of each of the additional target polypeptides is under the control of respective additional promoters.

211. The polynucleotide of claim 209, wherein the polynucleotide further comprises one or more DNA insulator elements, wherein said one or more DNA insulator elements are positioned between the 3′UTR of the PP7 bacteriophage coat protein transcript coding sequence and the second promoter, and wherein said DNA one or more DNA insulator elements enhance efficiency of termination of transcription of the PP7 bacteriophage coat protein.

212. The polynucleotide of claim 210, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

213. The polynucleotide of claim 211 or claim 212, wherein said DNA insulator element comprises CCCTC-binding factor site(s).

214. The polynucleotide of any one of claims 201-213, wherein the first promotor is an RNA polymerase II promoter.

215. The polynucleotide of claim 214, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

216. The polynucleotide of any one of claims 209-215, wherein the second promoter is an RNA polymerase II promoter.

217. The polynucleotide of claim 216, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

218. The polynucleotide of claim 202, wherein the first promoter is a bi-directional promoter which drives expression of the PP7 bacteriophage coat protein in one direction and the first target polypeptide in the opposite direction.

219. The polynucleotide of claim 218, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

220. The polynucleotide of any one of claims 2-3, 7-30, and 199-219, wherein the PP7 bacteriophage coat protein comprises the amino acid sequence of SEQ ID NO: 39, or a sequence having at least 70% sequence identity thereto.

221. The polynucleotide of any one of claims 199-220, wherein the nucleotide sequence encoding the PP7 bacteriophage coat protein further encodes a degron sequence.

222. The polynucleotide of claim 221, wherein the degron sequence is operably linked to the C-terminal end of the PP7 bacteriophage coat protein in the resulting protein.

223. The polynucleotide of claim 222, wherein the degron sequence is operably linked to the N-terminal end of the PP7 bacteriophage coat protein in the resulting protein.

224. The polynucleotide of any one of claims 221-223, wherein the degron sequence comprises the amino acid sequence of SEQ ID NO: 17, or a functional variant or ortholog having at least 70% sequence identity thereto.

225. The polynucleotide of any one of claims 199-224, wherein the first PP7 responsive RNA element is positioned in the 5′ UTR or the 3′ UTR of the nucleotide sequence encoding a first target polypeptide.

226. The polynucleotide of any one of claims 199-225, wherein when one or more additional combinations of a PP7 responsive RNA element and a nucleotide sequence encoding a target polypeptide are present, each additional PP7 responsive RNA element is positioned independently in the 5′ UTR or the 3′ UTR of the nucleotide sequence encoding the respective additional target polypeptide.

227. The polynucleotide of any one of claims 3, 7-30, and 199-226, wherein the PP7 responsive RNA element comprises the nucleotide sequence of SEQ ID No: 40, or a sequence having at least 70% sequence identity thereto.

228. A polynucleotide comprising:
a) a nucleotide sequence encoding a U1 small nuclear ribonucleoprotein A (U1A);
b) one or more microRNA (miRNA) Silencing Element (MSE) sequences, wherein each of said one or more MSE sequences is targeted by a miRNA, and wherein each of said one or more MSE sequences is positioned with respect to the nucleotide sequence encoding the U1A to allow expression of the ULA in the absence of the respective miRNA targeting said MSE sequence;
c) a first U1A responsive RNA element;
d) a nucleotide sequence encoding a first target polypeptide, wherein the first U1A responsive RNA element is positioned with respect to the nucleotide sequence encoding the first target polypeptide to allow translation of the first target polypeptide in the absence of the ULA; and
e) optionally one or more additional combinations of a U1A responsive RNA element and a nucleotide sequence encoding a target polypeptide, wherein within each combination, the ULA responsive RNA element is positioned with respect to the nucleotide sequence encoding the target polypeptide to allow translation of the target polypeptide in the absence of the U1A, wherein translation of each additional target polypeptide is controlled by its closest U1A responsive RNA element in the resulting mRNA.

229. The polynucleotide of claim 228, wherein the one or more MSE sequences are positioned in the open reading frame (ORF) or in the 3′ untranslated region (3′ UTR) of the U1A transcript coding sequence.

230. The polynucleotide of claim 228 or claim 229, wherein the polynucleotide further comprises a first promoter, and wherein expression of the ULA is under the control of the first promoter.

231. The polynucleotide of claim 230, wherein expression of the first target polypeptide is under the control of the first promoter.

232. The polynucleotide of claim 231, wherein the polynucleotide is structured as an alternatively spliced construct, and wherein one transcript comprises the nucleotide sequence encoding the U1A and one or more MSE sequences, and another transcript comprises the first U1A responsive RNA element and the nucleotide sequence encoding the first target polypeptide.

233. The polynucleotide of claim 232, wherein the polynucleotide further comprises a 5′ donor splice site and a 3′ acceptor splice site.

234. The polynucleotide of claim 233, wherein the 5′ donor splice site comprises the sequence AG*GU(A/G)AGU or AG*GU(A/G)GAU, wherein * denotes the splice junction.

235. The polynucleotide of claim 233 or claim 234, wherein the 3′ acceptor splice site comprises a branch point, a pyrimidine tract, and the sequence AG*G, wherein * denotes the splice junction.

236. The polynucleotide of any one of claims 228-235, wherein the polynucleotide comprises one or more additional combinations of a U1A responsive RNA element and a nucleotide sequence encoding a target polypeptide, and wherein expression of the first target polypeptide and the one or more additional target polypeptides is under the control of the first promoter.

237. The polynucleotide of any one of claims 228-236, wherein the polynucleotide comprises one or more additional combinations of a U1A responsive RNA element and a nucleotide sequence encoding a target polypeptide, and wherein expression of the first target polypeptide is under the control of the first promoter, and expression of each of the additional target polypeptides is under the control of respective additional promoters.

238. The polynucleotide of claim 230, wherein the polynucleotide further comprises a second promoter, wherein expression of the first target polypeptide is under the control of the second promoter.

239. The polynucleotide of claim 238, wherein the polynucleotide comprises one or more additional combinations of a U1A responsive RNA element and a nucleotide sequence encoding a target polypeptide, and wherein expression of each of the additional target polypeptides is under the control of respective additional promoters.

240. The polynucleotide of claim 238, wherein the polynucleotide further comprises one or more DNA insulator elements, wherein said one or more DNA insulator elements are positioned between the 3′UTR of the U1A transcript coding sequence and the second promoter, and wherein said DNA one or more DNA insulator elements enhance efficiency of termination of transcription of the U1A.

241. The polynucleotide of claim 239, wherein the polynucleotide further comprises one or more DNA insulator elements, wherein said DNA insulator elements are positioned between the 3′UTR of each subsequent transcript and each subsequent promoter, and wherein said DNA insulator elements enhance efficiency of termination of transcription.

242. The polynucleotide of claim 240 or claim 241, wherein said DNA insulator element comprises CCCTC-binding factor site(s).

243. The polynucleotide of any one of claims 230-242, wherein the first promotor is an RNA polymerase II promoter.

244. The polynucleotide of claim 243, wherein the first promoter is a cytomegalovirus (CMV) promoter, a simian virus 40 (SV40) promoter, a human elongation factor 1 alpha (EF1α) promoter, or a phosphoglycerate kinase (PGK) promoter.

245. The polynucleotide of any one of claims 238-244, wherein the second promoter is an RNA polymerase II promoter.

246. The polynucleotide of claim 245, wherein the second promoter is a cytomegalovirus (CMV) promoter, a simian virus 40 (SV40) promoter, a human elongation factor 1 alpha (EF1α) promoter, or a phosphoglycerate kinase (PGK) promoter.

247. The polynucleotide of claim 231, wherein the first promoter is a bi-directional promoter which drives expression of the ULA in one direction and the first target polypeptide in the opposite direction.

248. The polynucleotide of claim 247, wherein the bi-directional promoter is selected from human promoter for 3-phosphoglycerate kinase (hPGK) promoter, a minimal Cytomegalovirus (CMV) promoter, TRE3G promoter, Ponasterone A, and combinations thereof.

249. The polynucleotide of any one of claims 2-3, 7-30, and 228-248, wherein the U1A comprises the amino acid sequence of SEQ ID NO: 41, or a sequence having at least 70% sequence identity thereto.

250. The polynucleotide of any one of claim 228-249, wherein the nucleotide sequence encoding the U1A further encodes a degron sequence.

251. The polynucleotide of claim 250, wherein the degron sequence is operably linked to the C-terminal end of the ULA in the resulting protein.

252. The polynucleotide of claim 250, wherein the degron sequence is operably linked to the N-terminal end of the ULA in the resulting protein.

253. The polynucleotide of any one of claims 250-252, wherein the degron sequence comprises the amino acid sequence of SEQ ID NO: 17, or a functional variant or ortholog having at least 70% sequence identity thereto.

254. The polynucleotide of any one of claims 228-253, wherein the first U1A responsive RNA element is positioned in the 5′ UTR or the 3′ UTR of the nucleotide sequence encoding a first target polypeptide.

255. The polynucleotide of any one of claims 228-254, wherein when one or more additional combinations of a U1A responsive RNA element and a nucleotide sequence encoding a target polypeptide are present, each additional U1A responsive RNA element is positioned independently in the 5′ UTR or the 3′ UTR of the nucleotide sequence encoding the respective additional target polypeptide.

256. The polynucleotide of any one of claims 3, 7-30, and 228-255, wherein the U1A responsive RNA element comprises the nucleotide sequence of SEQ ID No: 42, or a sequence having at least 70% sequence identity thereto.

257. The polynucleotide of any one of claims 1-256, wherein the polynucleotide comprises two, three, or four MSE sequences.

258. The polynucleotide of any one of claims 1-257, wherein when two or more MSE sequences are present, at least two of these MSE sequences are targeted by the same miRNA.

259. The polynucleotide of any one of claims 1-257, wherein when two or more MSE sequences are present, at least two of these MSE sequences are targeted by different miRNAs.

260. The polynucleotide of any one of claims 1-259, wherein the one or more MSE sequences are targeted by miRNA(s) which is characterized by tissue-specific, cell type-specific, cell-state-specific or species-specific expression.

261. The polynucleotide of claim 260, wherein the one or more MSE sequences is targeted by miR-1, miR-155, miR-124, miR-182, miR-96, miR-183, miR-21, miR-16, miR-17, miR-19, miR-25, miR-34, miR-92, miR-93, miR-142, miR-222, miR-149, miR-375, miR-503, or miR-181b-2.

262. The polynucleotide of any one of claims 1-261, wherein the first target polypeptide is a gene editing nuclease, a chimeric antigen receptor (CAR), an antibody, a cytotoxic protein, a cell surface receptor, a transcription factor, an enzyme, a reporter protein, a cytokine, a viral protein, or a polypeptide that confers drug resistance.

263. The polynucleotide of any one of claims 50-53, 56 and 58, wherein the second target polypeptide is a gene editing nuclease, a chimeric antigen receptor (CAR), an antibody, a cytotoxic protein, a cell surface receptor, a transcription factor, an enzyme, a reporter protein, a cytokine, a viral protein, or a polypeptide that confers drug resistance.

264. The polynucleotide of any one of claims 1-263, wherein the polynucleotide encodes one or more additional target polypeptides, and wherein each additional payload polypeptide is independently a gene editing nuclease, a chimeric antigen receptor (CAR), an antibody, a cytotoxic protein, a cell surface receptor, a transcription factor, an enzyme, a reporter protein, a cytokine, a viral protein, or a polypeptide that confers drug resistance.

265. The polynucleotide of any one of claims 262-264, wherein the gene editing nuclease is a Cas protein, ZFN nuclease, or TALEN.

266. The polynucleotide of claim 265, wherein the Cas protein is Cas9.

267. The polynucleotide of any one of claims 1-266, wherein the polynucleotide is an RNA molecule.

268. The polynucleotide of any one of claims 1-266, wherein the polynucleotide is a DNA molecule.

269. A vector comprising the polynucleotide of any one of claims 1-268.

270. The vector of claim 269, wherein the vector is a viral vector.

271. The vector of claim 270, wherein the viral vector is a retroviral vector, an adenovirus vector, a parvovirus vector, an influenza virus vector, a herpes virus vector, a poxvirus vector, a rhabdovirus vector, a paramyxovirus vector, a morbillivirus vector, or a reovirus vector.

272. The vector of claim 271, wherein the retroviral vector is a lentiviral vector.

273. The vector of claim 271, wherein the parvoviral vector is an adeno-associated virus (AAV) vector.

274. The vector of claim 271, wherein the paramyxovirus vector is a Sendai virus (SeV) vector.

275. A composition comprising the polynucleotide of any one of claims 1-266 or the vector of any one of claims 269-274 and a carrier or excipient.

276. The composition of claim 275, wherein the composition is a pharmaceutical composition comprising the polynucleotide or the vector and a pharmaceutically acceptable carrier or excipient.

277. A host cell comprising the polynucleotide of any one of claim 1-266, or the vector of any of claims 269-274.

278. A method of selectively expressing a payload polypeptide in a target cell type or tissue or a cell in a specific state within a multicellular organism, comprising introducing to the multicellular organism an effective amount of the polynucleotide of any one of claims 1-266, or the vector of any one of claims 269-274, or the composition of claim 275 or 276.

279. The method of claim 278, wherein the multicellular organism is an animal, a plant, or a fungus.

280. The method of claim 279, wherein the animal is a human, veterinary animal, or experimental animal.
